SEQUENCE LISTING (646 sequences)

SEQ ID NO: 1 - protein, 455 aa, synthetic construct
  REGION 1..455: note = CasM. 298706 Effector Protein
MAKKGTNRKKMIVKVMKYELKYESGCADFNEMQNELWKLQRQTREVMNRTIQLCYHWSYVQADYCKQHGCARRDVKPCDVYETNATSLDGYIYQLFKDEYPNFLMANLIATLRKAHQKYDALLFDIQEGNSSIPSFKKDQPLIFSKEAIRLPECLSDKRQITLFCFSKPYKSAHPTLDKITFAVRARSASEKSIFDHIISGKYALGESQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKALESEKPYLKHWTYFDLQSKIIYKAEEKGIRVVKVNPKCTSRRCSACGYISKENRKNQVEFLCVNCGYHHNADYNAAQNLSIPQIDRLIEKQLKEQESEENEAGANPK

SEQ ID NO: 2 - protein, 410 aa, synthetic construct
  REGION 1..410: note = CasM. 280604 Effector Protein
MAKGTLSKVMKYELRYLDGCGDFQNMQKELWTLQRQSREILNRTIQIAYHWDYTDREQFKKTGQHLDIKAETGYKRLDGYIYDSLKEDVQNFASVNVNATIQKAWAKYKSSKIDVLRGDMSLPSYKSDQPLVLHAQSMKIFSSDDDDVLQVTLFSNAYKKACNYSNIRFIIGLHDATQRTIIKKVLSGDWGIGQSQIVYKRPKWFLYLTYNFSPEQHEVNPDKILGVDLGESIAIYASSIGEYGSLRIEGGEISAFAKQLEARKRSLQKQAAYCGKGRIGHGTKSRVSDVYKMEDKIANFRNTVNHRYSKMLIDYALKHMYGTIQMEDLSGIKKETGFPKFLQHWTYYDLQQKIEAKAKEHGINFIKVDPAFTSQRCSKCGNIDSENRPSQAVFCCKKCGYKTNADFNAS

SEQ ID NO: 3 - protein, 426 aa, synthetic construct
  REGION 1..426: note = CasM. 281060 Effector Protein
MNVTKVMRYQLIYQGGGGDFESLQNQLWEFQRQTRAILNKTIQTMYLATANQEKFSEKALYHDLCAEYPDMISSTVNATLREATKKYRSSVREILAGRMSLPSYKRDHPILLHNQSVALKQGNQGSYFATISVFSRKYQQGTPGVKQPSFQLIAKDNTQRTILQRLLSGEYKLGQCQLIYIRPKWFLNVAYSFTPSEKALDQEKVLGVDLGCVYAIYASSYGNHGIFKISGDEITSFERKQAAIQNRAFKNDLTRIREIEERRKQKLEQARYCGEGRIGHGVKTRVAPAYQDEGKISRFRETINHRYSKALVDYAEKNGYGTIQMEDLSGIKSSTGFPKRLQHWTYFDLQQKIKYKAEEQGIKVVKIKPAYTSQRCSRCGHIDPANRKSQSEFKCIACGFSSNADYNASQNISMRNIEKIIQGKAN

SEQ ID NO: 4 - protein, 432 aa, synthetic construct
  REGION 1..432: note = CasM. 284933 Effector Protein
MAKGTITKVMKYELRYLGGFSDFHEMQKEVWQLQRQYREILNKTIQIALHWDYVSAQQFGESGTYLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 5 - protein, 435 aa, synthetic construct
  REGION 1..435: note = CasM. 287908 Effector Protein
MSKGMLTKVMKYTLRYVGGCGDFHEMQSILWELQKQTRAVLNKTIQIAFEWDYRSREAFQETGEYLDVHAETGYKRLDGYIYNCLKNEYADFAGKNLNAAIQTAWKKYNQSKRDIQTGKMSLPSYRSNQPLIIHNDNVMISQDMQAAPSVRFTLLSLEYKKAHDLNTNPTFEVLINDGTQRAIFEKVRSGEYKLGQCMIQYDKKKWFLLLTYSFQPEKLTLDKNKILGVDLGETIVICASSVSERGRFVIDGGEITRFATQIEARKRSQQHQAAYCGEGRIGHGTKTRVDAVYKTEDRIANFRDTINHRYSRALVNYAVKHGFGTIQMEDLSGIKSSDDFPKFLRHWTYYDLQSKIESKAKERGIAVVKVNPRFTSRRCSKCGYIDEGNRKDQAHFCCLSCGFRANADFNASQNLSIKGIDKIIEKEYNANSKQT

SEQ ID NO: 6 - protein, 442 aa, synthetic construct
  REGION 1..442: note = CasM. 288518 Effector Protein
MGKPITKTMKYQIHYIDGCGDFHNMQKELWDLQRIVRQILNKTINESYLWFVRSEQYYRDTGENLSVEEQTGYKTLDGHIYNLLKQEYTQKLVSNSLNASIQAAYKKMKDSRRDVMIGTMSLPSYRSDQPIIIYNKNIKFSSHPEHGFVVDCSLFSDAYKKSQGYEKSVKFQVSVDDNTQRSIFENILTGNYKHGQCSIVYEKKKWFLLLTYSFVPEETKLDPDKILGVDVGVVYALYASSKGNHGTFKIKGDEAITFIQRVEARKHSRQLQGTYCGDGRIGHGTKTRVQPVYNERALISNFQDTINHRYSKALIDYAKKNGYGTIQMEDLSGIKEVQQYPKYLQHWTYYDLQLKIQYKAKEAGIGFVKVTPKYTSQRCSHCGNIDEANRPKQDVFRCTVCGYERNADYNASQNLSIKGIDRIIDDQLKQMNKANPKKTENA

SEQ ID NO: 7 - protein, 435 aa, synthetic construct
  REGION 1..435: note = CasM. 293891 Effector Protein
MSGGAITKVMKYDLTYKDGYGNFKDMQEAVWKLIRDTRTILNETIKIAYHWDYLNEKSKRETGEHLDLLEETGYKRLDGYIYDDLKDRFPDFASSNLNAAIQTAWKKYKQSQKDVYIGKMTLPSYKSDQPLPINKQSIKIYDEEREHIVELNLFSTKHKKEHGLASNVRFRINLHDNTQHAIYERVLSGEYTLGQCQLLYDRPKWFFILTYSFKPAQNKLDPDKILGVDMGETCALYASTFGEQGSFVINGGEVSEYAKREEARKRSLQKQAAVCGEGRIGHGTKTRVSSVYKEQERISNFRDTINHRYSKALIEYAVKNGCGTIQMEDLSGIRQSTDFPKFLRHWTYYDLQQKIKTKAKETGIAVSMIDPRYTSQRCSRCGHIDKANRKDQAHFHCLKCGYSCNADFNASQNISIRGIDKIIQKELGAKAKQTD

SEQ ID NO: 8 - protein, 431 aa, synthetic construct
  REGION 1..431: note = CasM. 294270 Effector Protein
MKEIAKVMKYQLIYLDGGGDFYELQQTLWDLQRQTREILNKTIQSMYLATATNTAFEENALYHRFGAEYPMMAALNVNATLRTAKKRYTSTIKETLRGTMSLPSYKRDQPILLHNQTIHLALEDGQYSALFSVYSEKFQKAHEGVARPRFALMARDGTQRAILDRLLDGSYRLGQSQMTYEQKKWFLSLTYKFVPEVRELDKSKILGVDLGCVYAIYASSMQQKGIFKISGDEITEFEKRQAAMQNREPVSTLERVEQLEQRRWQKQQQARYCGEGRVGHGTGTRVAPAYRDADKIARFRDTINHRYSKALVEYAEKNGFGTIQMEDLSGIKEDTGFPKRLRHWTYFDLQTKIQYKAAERGITVVKIDPQYTSQRCSRCGYIDKANRASQEKFLCQSCGFEANADYNASQNISVEKIDKLIAKDKKKLART

SEQ ID NO: 9 - protein, 436 aa, synthetic construct
  REGION 1..436: note = CasM. 294491 Effector Protein
MGQVTKVMRYQLIYQDGGGDFYTVQQELWELQRQTREILNKTIQTMYLADANKEKFDNAAERTLNRRFCVDHPDMYTKTVTATLRKAKAKYNASQKEILAGRMSLPSYKRDQPILLNPQGFKIEEESDSFFAAIAVFSDKYKNKHPDVDVKRLRFRLVVKDGTQRAIIRRVISGEYKLGRSQLLYSKKKWFLNVTYSFEPAEKKVDPDKILGVDLGCVYAIYASSFGSPGVFKISGDEVSSFERKQAAIQNRSPKSTLERVEKIEERHKQKQQQARYCGEGRIGHGTKTRIAPVYQDEDKIARFRDTVNHRYSKALIDYAEKNGYGTIQMEDLSGIKSATGFPKRLKHWTYYDLQTKIEYKAEERGIKVVKIDPRYTSQRCSRCGYIDSGNRKSQAEFCCMACGFSCNADYNASQNISIGGIAKIIADKRKEADAK

SEQ ID NO: 10 - protein, 368 aa, synthetic construct
  REGION 1..368: note = CasM. 295047 Effector Protein
YLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 11 - protein, 437 aa, synthetic construct
  REGION 1..437: note = CasM. 299588 Effector Protein
MAEKTIVKVMKFELRYIDGAGEFSEMQKHLWELQKQTREVLNKTIQMGYALECKRFAHHDKTGQWLDDKELTGSKYKAVADYINAELKEDYNIFYSDCRNSTVRKAYKKFKDAKNKIFSGEMSLPSYRSNQPIIIHNRNVIIRGNAESALVGLKVFSDGFKALHGFPAAVNFKLCVKDGTQRAIIENVISEIYKISESQLIYDNKKWFLILAYRFTQKKNDLNPDKILGVDLGVKFAVYASSIGEYGSFRIKGGEVTEFIKRLEKRKKSLQNQATVCGDGRIGHGTKTRVADVYKARDKISNFQDTINHRYSRAIVDYARKNGYGTIQLEKLDNSIEKKGDYSPVLVHWTYYDLRTKMEYKAAEYGIKVIAVEPKYTSQRCSKCGYISSENRKTQESFECIKCGYKCNADFNASQNLSVRDIDRIIDEYLGANPELT

SEQ ID NO: 12 - protein, 434 aa, synthetic construct
  REGION 1..434: note = CasM. 277328 Effector Protein
VVNVAKGALSKVMKFELSYLDGCGDFQNMQKELWTLQRQTREILNRTIQIAYHWDYTDREHFKKTGQHLDVKSETGYKRLDGYIYDELKETVQNFASVNVNATIQKAWAKYKSSKTDVLRGDMSLPSYKSDQPLVLHAQSIKLSEDKDGPVLQVTLFSNAHKKACDYSNVRFAFRLHDATQRAIFKNVLSGEYGLGQSQIVYKRPKWFLYLTYNFSPEQHGLDPDKILGVDLGESIALYASSLGDYGSLRIEGGEVTAFAKQLEARKRSLQKQAAHCGEGRVGHGTRARVSDVYKAEDKIANFRNTVNHRYSKKLIEYAIQNRYGTIQMEDLSGIKQDTGFPKFLQHWTYYDLQQKIEAKAKENGINFIKVDPSYTSQRCSKCGNIDSDNRPSQAVFCCTKCGFRANADFNASQNLSIPEIDKIIKKERGANTK

SEQ ID NO: 13 - protein, 455 aa, synthetic construct
  REGION 1..455: note = CasM. 297894 Effector Protein
MAKKGTNRKKMIVKVMKYELKYEKGCADFNEMQNELWKLQRQTREVMNRTVQLCYHWNYVQADYCKQHGCAHRDVKPCDVYETNATSLDGYIYQLFKDEYPNFLMANLIATLRKAHQKYDALLPDIQEGNSSIPSFKKDQPLIFSKEAIHLPECLSDKRQITLFCFSKPYKSAHPTLDKITFAVRAHSASEKSIFDNIINGKYALGTSQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKAMESEKPYLKHWTYFDLQSKIIYKAEEKGIRVVKVNPKCTSRRCSACGYISKENRKNQAEFLCVNCGYHHNADYNAAQNLSIPQIDRLIEKQLKEQESEESEAGANPK

SEQ ID NO: 14 - protein, 500 aa, synthetic construct
  REGION 1..500: note = CasM. 291449 Effector Protein
MTERHDNESSKIKAEVSLLNSSVPDFEKKRHVKVLKLHILKPAGDMKWDELGALLRDARYRVFRLANLAISEAYLDFHKWRSGGNEQPKLKISQLNRNLRSMLEDEVTGKQTKMIKSDRYSKSGALPDSIVSPLSMYKLGGLTSKSKWSEVLRGKSSLPTFKLNMAIPVRCDKPGDRRIERTKNGDAEVELRICLQPYPRVIIATGRNSLGDGQRAILDRLLDNTKYSEQGYRQRCFEIKEDQRSGKWHLFVTYDFPAIEPAKNLSRERIVGVDLGAACPLYAAINTGHARLGWKHFSPLAARVRALQNQTIRRRRQILRGGKVSLSEDSARSGHGRKRKLKPISKLEGKIDRAYTTLNHQLSATVIKFAKDNGAGVVQMEDLKGLRETLTGTFLGERWRYEELQRFIRYKADEAGIEIRLVNPQYTSRRCSECGHIHKDFTREFRDKSREGNKSVRFLCPDCGFTADPDYNAARNLASLDIAAIIERQLEIQGLRKHDP

SEQ ID NO: 15 - protein, 471 aa, synthetic construct
  REGION 1..471: note = CasM. 297599 Effector Protein
MKEKSKTLVKVARLRILKPAGDMKWSELGEMLRTVRYRVFRLANLAVSEAYLGFHMYRTNRATEFKAETIGKLSRRLREMLIEEGVDEKDLSRYSQTGAVPDTVAGALGQYKIRGITSPTKWRQVVRGQAALPTFRNDMAIPIRCDKQYQRRLEKTEAGEIEVELMICRKPYPRIVLGTADLGPGQRAILERLLQNTDNSADGYRQRLFEAKQDTQTKKWWLYVTYDFPRLKEGKLNQEIVVGVDLGFSIPLYVALNIGHARLGRRHFQALGNRIRSLQRQVLARRRSIQRGGRVNISHSTARSGHGRKRKLLPTEKLRGRIEKSYSTLNHQLSASVIDFAKNHHAGTIQIEDLANLKEELAGTFIGARWRYHQLQQFLKYKAEEAGITLNQVNPRYTSRRCSECGFINIDFDRAFRDAGRTEGRVTKFLCPECGYEADPDYNAARNISILDIDKLIRVQCKKQGLTYDAH

SEQ ID NO: 16 - protein, 475 aa, synthetic construct
  REGION 1..475: note = CasM. 286588 Effector Protein
MPERPKTVNKVIWFQIHKPAGDMTWKELGNLLREARYRVFRLANLAVSEKYLSFHMWRTGQEYKSETIGKLNRRLREMLIEEGVEEESQKRFSATGALPDTVVSTLAKGKLAAITSKSKWKDVVNGKTSLPTFKLNMAIPVRCDKAEQRRLRRTESGDVELELMICKQPYPRVVLKTGKLKSGQRAILDRLVENNDNSKEGYSQRVFEIKQVENNDGSKEWRLYISYTFPKKAVEANADVAVGVDIGFSVPLVAAVNNGLERLGYNDFRALNERIRSLQRQVLVRRRSMQSGGRDYVSTPTARSGHGRKRKLLPIQTLRKRWDNAYTTLNHQLSHAVVSFAENHGAATIQIENVKSLKDELRGTFLGQRWRYFELQQFLKYKADEVGIELREVNARYTSRRCSECGYINMAFTRQARDKGRVDGKPMEFVCPECGYKAHPDYNAARNIAMLDIEQKMQVQCKQQGITYADDSEVL

SEQ ID NO: 17 - protein, 448 aa, synthetic construct
  REGION 1..448: note = CasM. 286910 Effector Protein
MTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 18 - protein, 477 aa, synthetic construct
  REGION 1..477: note = CasM. 292335 Effector Protein
VGKEGKRNVKVMKIRILKPCDGMTWNELGQLLRDARYRVFRLANLTVSEAYLNFHLWRTGRSQEFKKQTIGQLNRQLRNILQQEKYDDEKLNRYSKTGALPDTVCSALWQYKLMAVMKKSKWSEVIRGKSSLPTFRNDMAIPVRCDKPEQKRIEKTEQGQVEAALQVCVQPYPRVILGTHTLGDGQDAILKRLLDNQNQAIGGYRQRSFEIKYDEQKRWWLFITYDFPATEVATDKTIAVGVDLGVSVPLYAAVNNGPARLGRREFGGLGRRIRDLRNQTDARRRSIQRSGREGQSDDTARAGHGRKRKLLPIHILEGRLDKAYTTLNHQMSAAVIKFAAEQGAGIIQIENLAGLQDELRGTFIGGRWRYRQLQDFLKYKTQEMGIELRQVNPKYTSRRCSKCGFIHKDFDRDYRNRHSENGKPAQFVCPNPDCKYESDPDYNAARNLATLDIEEQIRVQCQKQGLEYDSKKDKNAL

SEQ ID NO: 19 - protein, 472 aa, synthetic construct
  REGION 1..472: note = CasM. 293576 Effector Protein
MKEKSKTLVKVARLRILKPAGDMTWSELGEMLRTVRYRVFRLANLAVSEAYLGFHMFRTQRAAEFKAETMGKLSRRLREMLIEEGVDEKELNCYSLTGAVPDTVAGALHQYKIRGITSPTKWRQVVRGQAALPTFRNDMSIPIRCDKPYQRRLEKTEAGEVEVELMICRKPYPRIVLGTADVGPGQEVILERLLQNKDNSSDGYRQRLFEAKQDRQTGKWWLYVTYDFPRPEEGELNPEIVVGVDLGFSVPLYVAINNGYARLGRRHFQALGNRIRSLQRQVLARRRSIQRGGRVNISHDTARSGHGIKRKLLPTEKLRGRIEKSYSTLNHQLSASVIDFTKNHHAGTIQIEDLANLKEVLAGTFIGARWRYHQLQQFLKYKADEAGITLKEVNPRYTSRRCSECGFIHKDFDRAFRDSGRTDGKVARFVCPECGYGPVDPDYNAAKNISTLDIEKHIRVQCKKQGLEYEVH

SEQ ID NO: 20 - protein, 470 aa, synthetic construct
  REGION 1..470: note = CasM. 294537 Effector Protein
MKEKAKTLVKVARLRILKPAGDMTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 21 - protein, 468 aa, synthetic construct
  REGION 1..468: note = CasM. 298538 Effector Protein
MAKKAKTMFKVTNFRILKPAGDMTWKELGQLLRDARYRTFRMANLALSEAYLNFYLLKKGDLKEYKNVKIGQIAKRLRDMLIEEGVDEEVQNRFSPKVALPAYVYSALDQFKLRGLTSKSNWKKVLRGQASLPTFRLNMSVPIRCDKPEHRRLEKTENGNVEVDLMICRKPYPRVVLETLKLDGSSKAILDRLLENEDNSPGNYRQRCFEVKQNPRSNDWWLYVTYEMPVDKDKKLDPKVIVGVDLGFSVPLYVAINNGHARLGRRHFQALGKRIHNLQNQVLARRRSIQRGGQVNLSHSTSRSGHGRKRKLQPTEKLQQKINSAYSTLNHQLSSSVIDFANNHKAGTIQIEDLETLKEQLTGTYIGRQWRYYQLQQFIEYKAKENSITVKKINPKYTSRRCSMCGHIHADFDRTFRDRSSNKGFVTKFICPECNFEADPDYNAAKNISTLDIENKIKLQCKKQKIDY

SEQ ID NO: 22 - protein, 457 aa, synthetic construct
  REGION 1..457: note = CasM. 19924 Effector Protein
MPKITRKIELLFDRSGLSEEECKEKWRFIYQINDNLYRVANRLVNQLYLADEIDDILRLSDQEYIALRKKLANKKLDEATRISLEEQMSQVMKRVNERRSAILQRPQQSFAYSVVTDSDTEGLTAKILDVLKQDVLSHYKADTKEVLKGEKSISNYKKGMPIPFAFNDSLRLYKEDGFFYLKWYNGIRFLLNFGRDASNNQLIVERCLGISKDEISYKACSSSIQIKKKGNHSKIFLLLVVDVPVEQYAQKPNMVVGVDLGLNVPIYAASNSTLERKAIGSREAFLNQRGAFQRRFRALQRLQTTKGGRGRLHKLEPLERVREAERNWVRTQNHLFSREVINFAIDVGASTIQMEKLANFGRDAQGEVREDKKYVLRNWSYFELQNLIEYKAKRAGIKVKYINPAFTSQTCSECGQLGERDSIHFKCTNPDCPNCGKDIHADYNGARNIAKSKDYIK

SEQ ID NO: 23 - protein, 468 aa, synthetic construct
  REGION 1..468: note = CasM. 19952 Effector Protein
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 24 - protein, 468 aa, synthetic construct
  REGION 1..468: note = CasM.274559 Effector Protein
MPTITRKIELTLCTDGLSEEQRKEQWGLLYHINDNLYKAANNISSKLYLDDHVSSMVRMKHAEYLSLLKELARAEKQKTPDADAIAELKKKVAATEKEMTDQEHAICKYATEMSTQSLSYRFSTEFETKIFAKILDCLKQGVFATFNSDAKDVKRGERAIRNYKKGMPIPFAWTDSLRIKKDNKDFYLLWYNGLRFLFNFGKDRSNNRLIVERCLKMDADYDGEYKLCNSSIQIAKREGKVKLFLLLVVSIPKEHVELNKKVVVSVDLGINVPAYVATNITEERKAIGDREHFLNSRMAFQRRYKSLQRLKGTTGGKGRTKKLEPLERLRKAEHNWVHTQNHLFSREVVDFAVKTHAATIHMEDLSGFGKDNDGNADERKEFVLRNWSYYELQNMISYKAAKYGIKVEKIRPAYTSKTCSWCGQHGFREGVTFICENPACKQCGEKVHADYNAARNIANSKEIIKKNE

SEQ ID NO: 25 - protein, 469 aa, synthetic construct
  REGION 1..469: note = CasM. 286251 Effector Protein
MPTITRKIELTLCTEGLSDQERKDQWNLLYHINDNLYRAANNISSKLYLDDHVGSMVRLKHAEYLSLLRALEKAKKQKAPDEEVIAELSQQVATAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNCLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDNDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 26 - protein, 468 aa, synthetic construct
  REGION 1..468: note = CasM. 288480 Effector Protein
MPTITRKIELTLLTEGLSEEQRKEQWGLLYHINDNLYKAANNISSKLYLDDHVSTMVRMKHAEYLSLLRELARAEKQKKPDVDAIAELREKVTAAEKEMSDQERAICTYATEMSTQSLSYRFATEIETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIVKREGKVKLFLLLVVSIPQEHVELNKKIVVGVDLGINVPAYVATNITEERKAIGDREHFLNSRMAFQRRYKSLQRLKGTAGGKGRTKKLEPLERLRKAEHNWVHTQNHLFSREVVDFAVKSHAATIHMEDLSGFGKDNDGNADERKEFVLRNWSYYELQNMIAYKAAKYGIKVERIRPAYTSKTCSWCGQLGFREGVTFICENPECKQCGEKVHADYNAARNIANSKDIIKKNE

SEQ ID NO: 27 - protein, 463 aa, synthetic construct
  REGION 1..463: note = CasM. 288668 Effector Protein
MPTMTRKIELKLCTEGLSDEERKAQLGLLYHINDNLYKAANNISSKLYLDDHVSSMVRLKHAEYLSLLNEFEKAKKKGDEEQIVELSLRVAAAEKELTDQELAICKYATEMSTDTLAYRFANEIEINVFGQILACLKQGIHSTFKKDAADVKRGERAIRNFKKGMPIPFPWSKSIRIENEGSDFYLRWYNGLRFRFDFGKDRSNNRLIVSRCLNLDPDFEDEYKLSNSSLQMVKRDGRPKLFLLLVVNIPQENVELNKKIVVGVDLGINSPAYVATNITMERQRIGSRDTFLNARMAIQRRFQSLQKLQNTAGGRGRKKKLEPLERLKETERNWVRTQNHLFSRDVVQFAVKTRAATIHMEDLSGFGKDDDGNADEKKEFVLRNWSYYELQTMIKYKAAKYGIKVEKIRPAYTSRTCSWCGHEGDRKGETFICENPECEKYGKKENADYNAARNIANSTDIIK

SEQ ID NO: 28 - protein, 468 aa, synthetic construct
  REGION 1..468: note = CasM. 289206 Effector Protein
MPTITRKIELTLCTEGLSDEQRKEQWGLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNNSLKIESDSGEFYLRWYNGLRFLLTFGKDRSNNRMIVNRCMKMDEDFEGEYKLCNSSIQLAKRDGKPKLFLLLVVNIPQEHVKLNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDLSGFGKDKDGNADEKKEFVLRNWSFYELQNMIAYKSAKYGIKVVKIRPAYTSKTCSWCGQQGDRKSTTFICENPKCKHYGESIHADYNAARNIANSNDIVKENE

SEQ ID NO: 29 - protein, 467 aa, synthetic construct
  REGION 1..467: note = CasM. 290598 Effector Protein
MPKITRKIEMTLCTEGLSDEQRKEQWGLLYHINDNLYKAANNISTKLYLDEHVSSMVRMKHADYLSLLKELAKAEKKSPDEDLIAELREKLAAAEQEMTDQELAICKYATEMSTQTLAYKFATEIEINVFGQILACLKQAAQSNFKSDAKDVKRGERAIRNYKKGMPIPFPWNDNIRIDADGDEFYLRWYNGLRFHLTFGKDKSNNRMIVKRCLKMDKDFEGEYKLCNSSIQMVKRDGKPKLFLLLVVNIPQEHVELNKNVVVGVDLGVNVPAYVATNITEERKAIGEREHFLNTRMQIQRRYKSLQRLKATAGGKGRTKKLEPLERLRKAEHNWVHTQNHLFSREVVNFAVQTHAATIHMEDLSGFGKDDDGNADEQKEFVLRNWSFYELQNMIAYKAAKYGIKVEKVKPAYTSKTCSWCGQLGFRQGVTFICENPACKQCGEKVHADYNAARNIANSKDIIKKNE

SEQ ID NO: 30 - protein, 469 aa, synthetic construct
  REGION 1..469: note = CasM. 290816 Effector Protein
MPTITRKIELHLCTDGLTDEQQKAQRLLLYHINDNLYKAANNVSSKLYLDEHVSSMVRLKHDEYLSLSRELARAEKKHDDELTTELRGKLAAAEREMTDQELAICKYATEMSTQSLSYRLVTELETKIFAKILDCLKQGVYATFNSDARDVKRGERAIRNYKKGMPIPFAWNDSVRIEYDEKEKDFYLRWYNDIRFKFHFGRDRSNNRLIVSRCLKLDKDYEGDYQLCNSSIQIVKRDGSTKFFLLLVVKIPQEHVELNKRIVVGVDLGINYPAYVATNCTEERMYIGDREHFLNTRMQFQRRYKSLQKLKGTAGGKGRSKKLEPLERLRNAERNWVHTQNHLFSLKVVNFAVQTHAATIHLEDLSGFGKDDDGNADERKEFVLRNWSYYELQSMIEYKAKKYGIKVEKIRPAYTSQTCSWCGQRGFRQGVTFICENPECKKCGEKENADYNAARNIANSKDVIKDKNE

SEQ ID NO: 31 - protein, 491 aa, synthetic construct
  REGION 1..491: note = CasM. 295071 Effector Protein
TPFVLYFQNYSLSLRQHITLYSMPTITRKIELTLCTEGLSDQERKDQWNLLYHINDNLYRAANNISSKLYLDDHVGSMVRLKHAEYLSLLRAMEKAKKQKAPDEEVIAELSQQVAAAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNRLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDRDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 32 - protein, 469 aa, synthetic construct
  REGION 1..469: note = CasM. 295231 Effector Protein
MPTITRKIELHLCTEELSDEQQKAQRLLLYHINDNLYKAANNVSSKLYLDEHVSSMVRLKHDEYLSLLRELARAEKKADDELATQLREKLVAAEREMTDQELAICKYATEMSTQSLSYRFVTELETKIFAKILDCLKQGVYATFNSDSRDVKRGERAIRNYKKGMPIPFAWDKSVRIEYEEKEKDFFLRWYNDIRFKFHFGRDRSNNRLIVSRCMKLDKDYEGDYQLCNSSIQIVKRDGSTKYFLLLVVKIPQEHVELNKKIVVGVDLGINYPAFAATNCTEERMSIGDREHFLNTRMQFQRRFKSLQRLKGTTGGKGRNKKLEPLERLRKAEHNWVHTQNHLFSLKVVNFAVQAHAATIHLEDLSGFGKDDDGNADERKEFVLRNWSYYELQNMIKYKAKKFGIQVEKIRPAYTSQTCSWCGQRGFRQGITFICENPECKKCGEKENADYNAARNIANSKDIIKDKDE

SEQ ID NO: 33 - protein, 460 aa, synthetic construct
  REGION 1..460: note = CasM.292139 Effector Protein
MPIITRKIELHISKEGLSAEDYKAQWQYLRQINDNLYMAANRVSSHCFLNDEYKYRLCLQIPDYIDIEKQLKDSKRARLSKEELGQLKKRKKELENTVKGRFQDEFEKNSLYTIISNEFGEIIPGQILTCLRQCVQSKYNRAKEELEKGERAISTYKKGMPIPFPINKSIRLQKQGEDFVLKWYNKIVFKLHFGRDRSNNRVIVERLIQSALNDKQKGEDYVMNNSSIQLVEKDKMTKIFLLLSMDIPTQKRKLDSELVLGVDLGLNFPLYYATNQSANIHDHIGDKDIFLKERMVFQRRFKELQRLQCTQGGRGRKKKLEPLEKLRDKERNWVRTKNHIFSREVIKVALHLGAGTIHLENLHNFGKDGNGELKNSKKFVFRNWSYFELQSMIEYKAKMEGITVKYVNPAYTSQTCSVCGMIGERKEQAVFRCMNSSCLEYGKEVNADFNAARNIAKAKM

SEQ ID NO: 34 - protein, 466 aa, synthetic construct
  REGION 1..466: note = CasM. 279423 Effector Protein
MPTITRKIELTLCTDGLSDDLRKDQWQLLYHINDNLYKAANNISSKLYLDEHVASMVRLKHAEYLGLIKELAKARKRADDEAVRDLCSKLAVAEQEMNEQAKAICDYATEMSTQTLSYNFAKEIETNIFGQILTCLRQGVLLNFNSDARDVKRGERAIRNYKKGMPIPFPWNDTIKIVSEGDEFYLRWFSGLRFHLNFGKDRSNNRMIVRRCLKMEQDFDEEYKISNSSIQVAKRDGKQKLFLLLVVQIPQEQVVLNKKIVVGVDLGVNVPAYVATNITEERKAIGDREHFLNTRMQFQRRYKSLQRLKTTEGGRGRAKKLEPLERLRKAEHNWVHTQNHLFSREVVNFALQTQAATINMEDLSGFGKDNDGNADECKEFVLRNWSYYELQNMIVYKASKYGIRVQKIRPAYTSKTCSWCGHMGFREGVTFICENPDCKQFGEKVHADYNAARNIANSKEIIKNDE

SEQ ID NO: 35 - protein, 444 aa, synthetic construct
  REGION 1..444: note = CasM. 20054 Effector Protein
MSKTVTKTVKIALICEHTNKYGEKVDYKDINKLLWKLQKQTRELKNKTIQLCWEYNNFSCDYYKEHHEYPNMEDILKYKRINGFVENKLKTVNDLYSSNCSTTILSTCNEFQNYRSEFLKGTRSINSYKSDQPLDLHKGAIKLEHDGKDFYVSLKLLKRSAFNAMEFKGSDIRFKLNVKDKDKSTLKILESCYDKIYSISASKMTYDRKAGKWFLLLAYSFTPAKTENLDPEKILGVDLGIKIPICASVYGDLDRLTIEGGKIEEFRRRVEARKRSLQKQGKQCGDGRIGHGTKKRIKPITDIGDKIARFRDTENHIYSRYLIEYAVKKGCGTIQMEKLEGITREKDIFLKNWTYFDLQKKIEYKAKEKGIKVVYIEPAYTSKRCSSCGFIDTDNRLDQAHFKCLKCGFNENADYNASQNIGIKNIDKIIKEEHKSASDKLTSE

SEQ ID NO: 36 - protein, 439 aa, synthetic construct
  REGION 1..439: note = CasM. 282673 Effector Protein
VIILTKVVKLYLISEQINKEGQKIDYQRINSILWDLQKQTRDIKNRTVQLCWEWMNFSSDYCKTQEEYPKERDILGYTLEGYVYDYFKTGYDLYTGNISTSSREVCSSFKNVKKEILKGERSILSYKANQPLDLHKKAISLEYDNFNFFVKLKLLNRTGKKKYDITEDINFKIQVNDKSTRTILERCYDKEYKISGSKLIYEKKKKLWRLNLCYSFENSQVETLEKDKILGIDLGIVYPLMASIYGEYDRFSIKGGEIEEFRRRTEARKRSILQQTKYCGDGRIGHGRNKRTQPAYKINDKIARFRDTANHKYSRALIEYAVKKNCGIIQMENLTGISDNTDCFLKDWSYYDLQTKIENKAKEMGIKVVYIKAQYTSQRCSRCGYIDVNNRIRQALFKCQNCGYETNADYNASQNIGMYDIENIIEETLKIQSANVKQS

SEQ ID NO: 37 - protein, 437 aa, synthetic construct
  REGION 1..437: note = CasM. 282952 Effector Protein
MTKVTKVYLISEQIDKDGNKIDFKKISELLWNLQMQTRDIKNKCVQLCWEWLNFSSDYYKKSEEYPKEKDTLGYTLSGFVYDRIKNGSDLYSSNLSTSSRDTCTAFSNYKKEMLKGERSVLSFKANQPLDIHNKAIKLSYENGNFFVALKMLNRAGKEKYGIKDDLRFRMQVRDKSVRTILERLMNDEYKVSASKLMYDKKKKLWKLNLCYSFDNHVISTLDTEKIMGVDLGVVYPIMASVNGDYARFSIKGGEIEAFRSRVEARRRSLLNQSRYCGDGRIGHGRKKRTEPATQIADKIARFRDTTNHKYSRALIDYAIKNGCGTIQMEKLTGITSSAEHFLKEWSYFDLQTKIESKAKEAGIKVVYINPKFTSQRCNKCGYIHTDNRPVQARFCCQKCGYEENADYNASQNIGTKHIDVIIEETLKMQCEPETPTE

SEQ ID NO: 38 - protein, 428 aa, synthetic construct
  REGION 1..428: note = CasM. 283262 Effector Protein
MNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 39 - protein, 443 aa, synthetic construct
  REGION 1..443: note = CasM. 284833 Effector Protein
VTLLVKVVKIYLISEQFDKAGNQIDYKEVNKILWELQKQTREAKNKTVQLLWEWNNFSSDYVKASGIYPKAKDIFGYSSVHGQANKELRTKLALNSSNLSTTTMDVCKIFNTYKKEVWEGKRSVPSYKSDQPLDLHKESIKLIYENNEFYVRLALLKKAEFAKYGFKDGFRFKMQVKDNSTKTILERCFDEVYKINASKLLYDQKKKKWKLNLSYSFDNKNISELDKEKILGVDVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 40 - protein, 428 aa, synthetic construct
  REGION 1..428: note = CasM. 287700 Effector Protein
MNKVVKLALICEQSDKNNSPVDYKKVNEILWELQKQTREIKNKTIQYCWEYYNFSSDYYKKFNKYPKEKDILSYTLWGFINDKFKTGNDLYSGNCSATTKKVIKEFKNSKKELIRGSRSIINYKSNQPLNIHNKCIHLQFKNNNFYVSINLLNRRSFKKYNFANTAIKFKILVRDNSTKAILERCISNEYKISESQLIYNKKKKCWFLNLSYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKNNCGTIQMEDLTGITDNANRFLKNWSYYDLQTKIEYKAKEASINVVYINPENTSRRCSKCGYIDKENRKTQSSFICLKCGFKENADYNASQNISIKDIDKLIKEDVH

SEQ ID NO: 41 - protein, 443 aa, synthetic construct
  REGION 1..443: note = CasM. 291507 Effector Protein
VTLLVKVVKIHLISEQFDKAGNRIDYEEVNKILWELQKQTREAKNKTVQLLWEWNNFSSDYVKASGIYPKAKDIFGYSSVHGQANKELRTKLALNSSNLSTTTMDVCKNFNTYKKEVWKGKRSVPSYKSDQPLDLHKDSIKLIYENNQFYVRLALLKKAEFAKYGFKDGFHFKMQVKDNSTKTILERCFDEVYKINASKLLYDQKKKKWKLNLSYSFDNKNISELDKEKILGVDVGVSYPLVASVFGDRDRFKIKGGEIEKFRKSVEARRRSMLEQTKYCGDGRIGHGRKKRTEPALNIGDKIARFRDTTNHKYSRALIEYAVKKGCGTIQMEKLTGITSKADRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 42 - protein, 449 aa, synthetic construct
  REGION 1..449: note = CasM. 293410 Effector Protein
LIWKDALGGIILTKIVKLYLISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDKKEHFLKEWSYYDLQTKIENKAKEKGIKIVYINPEYTSQRCSKCGYIDANNRELRAVFKCQKCGFEADADYNASQNIGIKNIEDIIENTLKISSANEKQTKNT

SEQ ID NO: 43 - protein, 462 aa, synthetic construct
  REGION 1..462: note = CasM. 295105 Effector Protein
VFYSTFLCYILTKYIDFSANECYNINTSSEVKQLMNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 44 - protein, 430 aa, synthetic construct
  REGION 1..430: note = CasM. 295187 Effector Protein
LISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDNKEHFLKEWSYYDLQTKIENKAKEKGIKIVYINPEYTSQRCSKCGYIDANNRELRAVFKCQNCGFEADADYNASQNIGIKNIEDIIENTLKISSANEKQTKNT

SEQ ID NO: 45 - protein, 429 aa, synthetic construct
  REGION 1..429: note = CasM. 295929 Effector Protein
LVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 46 - protein, 707 aa, synthetic construct
  REGION 1..707: note = Cas Phi.1 Effector Protein
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 47 - protein, 757 aa, synthetic construct
  REGION 1..757: note = Cas Phi .2 Effector Protein
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 48 - protein, 765 aa, synthetic construct
  REGION 1..765: note = Cas Phi.3 Effector Protein
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 49 - protein, 766 aa, synthetic construct
  REGION 1..766: note = Cas Phi.4 Effector Protein
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 50 - protein, 812 aa, synthetic construct
  REGION 1..812: note = Cas Phi.5 Effector Protein
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 51 - protein, 812 aa, synthetic construct
  REGION 1..812: note = Cas Phi.6 Effector Protein
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 52 - protein, 793 aa, synthetic construct
  REGION 1..793: note = Cas Phi.7 Effector Protein
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 53 - protein, 441 aa, synthetic construct
  REGION 1..441: note = Cas Phi.8 Effector Protein
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 54 - protein, 812 aa, synthetic construct
  REGION 1..812: note = Cas Phi.9 Effector Protein
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 55 - protein, 812 aa, synthetic construct
  REGION 1..812: note = Cas Phi.10 Effector Protein
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 56 - protein, 761 aa, synthetic construct
  REGION 1..761: note = Cas Phi.11 Effector Protein
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 57 - protein, 717 aa, synthetic construct
  REGION 1..717: note = Cas Phi.12 Effector Protein
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 58 - protein, 781 aa, synthetic construct
  REGION 1..781: note = Cas Phi.13 Effector Protein
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 59-61 - skipped (3 entries)

SEQ ID NO: 62 - protein, 765 aa, synthetic construct
  REGION 1..765: note = Cas Phi.17 Effector Protein
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 63 - protein, 766 aa, synthetic construct
  REGION 1..766: note = Cas Phi.18 Effector Protein
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 64 - protein, 870 aa, synthetic construct
  REGION 1..870: note = Cas Phi.19 Effector Protein
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 65 - protein, 830 aa, synthetic construct
  REGION 1..830: note = Cas Phi.20 Effector Protein
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 66 - protein, 790 aa, synthetic construct
  REGION 1..790: note = Cas Phi.21 Effector Protein
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 67 - protein, 790 aa, synthetic construct
  REGION 1..790: note = Cas Phi.22 Effector Protein
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 68 - protein, 782 aa, synthetic construct
  REGION 1..782: note = Cas Phi.23 Effector Protein
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 69 - protein, 774 aa, synthetic construct
  REGION 1..774: note = Cas Phi.24 Effector Protein
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 70 - protein, 769 aa, synthetic construct
  REGION 1..769: note = Cas Phi.25 Effector Protein
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 71 - protein, 767 aa, synthetic construct
  REGION 1..767: note = Cas Phi.26 Effector Protein
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 72 - protein, 763 aa, synthetic construct
  REGION 1..763: note = Cas Phi.27 Effector Protein
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 73 - protein, 761 aa, synthetic construct
  REGION 1..761: note = Cas Phi.28 Effector Protein
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 74 - protein, 746 aa, synthetic construct
  REGION 1..746: note = Cas Phi.29 Effector Protein
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 75 - protein, 735 aa, synthetic construct
  REGION 1..735: note = Cas Phi.30 Effector Protein
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 76 - protein, 725 aa, synthetic construct
  REGION 1..725: note = Cas Phi.31 Effector Protein
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 77 - protein, 718 aa, synthetic construct
  REGION 1..718: note = Cas Phi.32 Effector Protein
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 78 - protein, 708 aa, synthetic construct
  REGION 1..708: note = Cas Phi.33 Effector Protein
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 79 - protein, 848 aa, synthetic construct
  REGION 1..848: note = Cas Phi.34 Effector Protein
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 80 - protein, 781 aa, synthetic construct
  REGION 1..781: note = Cas Phi.35 Effector Protein
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 81 - protein, 752 aa, synthetic construct
  REGION 1..752: note = Cas Phi.36 Effector Protein
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 82 - protein, 747 aa, synthetic construct
  REGION 1..747: note = Cas Phi.37 Effector Protein
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 83 - protein, 702 aa, synthetic construct
  REGION 1..702: note = Cas Phi.38 Effector Protein
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 84 - protein, 661 aa, synthetic construct
  REGION 1..661: note = Cas Phi.39 Effector Protein
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 85 - protein, 491 aa, synthetic construct
  REGION 1..491: note = Cas Phi.41 Effector Protein
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 86 - protein, 531 aa, synthetic construct
  REGION 1..531: note = Cas Phi.42 Effector Protein
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 87 - protein, 767 aa, synthetic construct
  REGION 1..767: note = Cas Phi.43 Effector Protein
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 88 - protein, 760 aa, synthetic construct
  REGION 1..760: note = Cas Phi.44 Effector Protein
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 89 - protein, 733 aa, synthetic construct
  REGION 1..733: note = Cas Phi.45 Effector Protein
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 90 - protein, 479 aa, synthetic construct
  REGION 1..479: note = Cas Phi.46 Effector Protein
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 91 - protein, 452 aa, synthetic construct
  REGION 1..452: note = Cas Phi.47 Effector Protein
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 92 - protein, 425 aa, synthetic construct
  REGION 1..425: note = Cas Phi.48 Effector Protein
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 93 - protein, 809 aa, synthetic construct
  REGION 1..809: note = CasM. 1584 Effector Protein
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 94 - protein, 840 aa, synthetic construct
  REGION 1..840: note = CasM. 1730 Effector Protein
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 95 - protein, 849 aa, synthetic construct
  REGION 1..849: note = CasM. 1816 Effector Protein
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 96 - protein, 779 aa, synthetic construct
  REGION 1..779: note = CasM. 1862939 Effector Protein
ELCKIDFTDARSSSLIEYFKFAINDNLKNDRGYLKAYVNDVEQIRADLKKVGGKQRNLEDRVSRTKVQLTLTNHIEDREGKQRVSRTERELIVPQNIKLYSQIVGREVKTTKEIYLIKRFLEYRSDLLFYYGFVDNFFKVEGNKKELCKIDFTDARSSSLIEYFKFAINDNLKNDRGYLKAYVNDVEQIRTDLQKVKTIFSKLRHALMHFDYDFFEKLFNGEEVGFDFDIKFLNIMIDKVEKLNIETKKEFIEDEVITLFGERLSLKKLYGLFSHIAINRVAFNKFINSFLIKDGIENRALKDFFNDEKGSQAYEIDIHSNAEYKALYVQHKKLVMATSAMSDGNEIAKKNQEISELKEKMNAITKANSLARLEYKLRLAFGFIYTEYGDYTAFKNSFDRDVKSAKYKELSVERLKAYYLATFKASKPQSHEKLEEVAKKIDRLSLKQLIENETLLKFVLLLFTFMPQELKGEFLGFIKKYYHDKKHIEQDTKEKEEEREGLSTGLKLKVLEKNIRSLSILKHALSFQVKYNKKDKNFYEEGNLHGKFYKKLAISHNQEEFNKSVYAPLFRYYVALYKLINDFEIYSLAQHIVNNETLADQVGKAQFRQRGYFNFRKLVNCTYATAQNSSYNVLIFMRNDISHLSYEPLFNCPLEEKASYKQKIRGREKIISVKPLSESRAEIVRFIASQTDMKKLLGYDAVNDFNMKMVQLRRRLSVYANKQETIEKMINKAKTPNDFYNLYKLKGIECINQHLLKVIGVTEAEKRIEKQIEEGNEKY

SEQ ID NO: 97 - protein, 802 aa, synthetic construct
  REGION 1..802: note = CasM. 1862895 Effector Protein
MLKKPSNRYALPKVILSTVDHEKILEFKVKYEKLARLDRLVVERMHFDGESVVFDEVIANSGDLEIAYQDDHRKLLIQAAGKSYTITGKKVGGKKRKLEERISRAKIQLTLTDGQEDQHRRIRATVTEKALLEPKEDRDIYSKISDRKIKTSKEIYLVKRFLSYRSDLLFYYFFVDNFFKVGNNKQELWKIKFQNQPELIEYFRFIINDRFKNAKNDKFDNYLKNDKAIQEDLEKIQKVFEKLRHALMHYDYGFFEKLFGGEDQGFDLDIAFLDNFVKKIDKLNIDTKKEFVDDEKIKIFGEDLNLADLYKLYASISINRVGFNRVVNEMIIKDGIEKSELKRAFEKKLDKTYALDIHSDPSYKKLYNEHKRLVTEVSTYTDGNKIKEGNQKIAKLKYEMKEITKKNALVRLECKMRLAFGLIYGRYDTHEAFKNGFDTDLKRGEFAQIGSEEAIGYFNTTFEKSKPKSKEEIKKIARQIDNLSLSTLIEDDPLMKFIVLMFLFVPRELKGEFLGFWRKYYHDIHSIDSDAKSDEMPDEVSLSLKLKILTRNIRRLNLFEYSLSEKIKYSPKNTQFYTDKSPYQKVYKRLKISHNKEEFDKTLLVPLFRYYSILFKLINDFEIYSLAKANPDASSLSELTKTKHGFRGHYNFTTLMMDAHKVSQGDSKKHFGIRGEIAHINTKDLIYDPLFRKSKMAQQRNDVIDFVLKYEKEIKAVLGYDAINDFRMKVVQLRTKLKVYSDKTQTIEKLLNEVEAPDDFYVLYKVKGVEAINKYLLEIVSVTQAEEEIERKIITGNKRYNT

SEQ ID NO: 98 - protein, 840 aa, synthetic construct
  REGION 1..840: note = CasM. 1862903 Effector Protein
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 99 - protein, 845 aa, synthetic construct
  REGION 1..845: note = CasM. 1862909 Effector Protein
MIKKPSNRHALPKVIISKVDNQNILEFKIKYKKLSRLDRVEIKTMHYDDRAIVFDEVIINGGLIDVEYRDNHKTIFVKVGDKSYSISGQKVGGKERLLENRISQTKVQLELKDEATNRVSKTERELIVDDNIKLYSQIVGRDVKTTKDIYLIKRFLGYRSDLLFYYGFVNNFFHVANNRPEFWKIDFNDNRNSKLIEYFIFTINDHLKNDENYLKDYISDRGQIVDDLENIKHIFSALRHGLMHFDYDFFEALFNGEDIDIKMDNQGNTQPLSSLNIKFLDIMIDKLDKLNIDTKKEFIDAEKITIFGEELSLAKLYRFYAHTAINRVAFNKLINSFIIENGVENQSLKEYFNQQAGGIAYEIDIHQNREYKNLYNEHKKLVSRVLSISDGQEIATLNQKIVELKEQMKQITKINSIKRLEYKLRLAFGFIYTEYKNYEEFKNSFDTDIKNGRFTPKDEDGNKRAFDSRELEHLKGYYKATLQTQKPQTDEKMEEVSKRVDRLSLKSLIGDDTLLKFILLMFTFMPQELKGEFLGFIKKYYHDTKHIDQDTISDSDDTIEEGLSIGLKLKILDKNIRSLSILKHSLSFQTKYNKKDRSYYEDGNIHGKFFKKLGISHNQEEFNKSVYAPLFRYYSALYKLINDFEIYTLSLHIVGNETLSDQVNKPQFLSGRYFNFRKLLTQSYNISNNSTHSVIFNAVINMRNDISHLSYEPLLDCPLNGKKSYKRKIRNQFRTINIKPLVESRKMIIDFITLQTDMQKVLGCDAVNDFTMKIVQLRTRLKAYANKEQTIEKMITEAKTPNDFYNIYKVKGVEAINKYLLEVIGETQVEKEIREEIERGNIANS

SEQ ID NO: 100 - protein, 822 aa, synthetic construct
  REGION 1..822: note = CasM. 1862917 Effector Protein
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 101 - protein, 832 aa, synthetic construct
  REGION 1..832: note = CasM. 1862921 Effector Protein
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 102 - protein, 806 aa, synthetic construct
  REGION 1..806: note = CasM. 1862947 Effector Protein
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 103 - protein, 835 aa, synthetic construct
  REGION 1..835: note = CasM. 1422 Effector Protein
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 104 - protein, 847 aa, synthetic construct
  REGION 1..847: note = CasM. 1740 Effector Protein
MMTKKPANRHALPKVIISEVDNTNILEFKIKYEKLARLDRVEVKAMHYEDGRIIFDEVVVNGGLIEVEYQDDHKTLFVQVGEKSYSISGQKVGGKQRLLEDRVSKTKVQLELSDGSSERVSRTERELIVADNIKLYSQIVGHEVKTTKEIYLAKRFLGYRSDLLFYYGFVDNFFRESKNLKYGKQPVELWEDKFQVNDKLTAYTKFMFNDDLQNSESYLKEYVKDNHKIKNDLESARDIFATFRHNLMHFNYSFFTRLFNGEDVKIKNLQTKKFESLSDVLRNVEFLNKVIQSIDKLNIDTRKEFIDKEKITLFNEELDLQQLYGFFAYTAINRVAFNKLINSFIIKDGIENEQLKEYFNQRVDGTAYEIDIHQNREYKELYKKHKNLVSKVSTLSDGKEIARGNTEISVLKEQMNKITKANSLKRLEHKLRLAFGFIYTEYGSYKAFVSRFNEDTKRKKIKNVEFEKIGVEKQKEYYESTFTSNNKDKLGELIQEYEKLSLNDLIENDTFLKVILLLFIFMPKEVKGDFLGFIKKYYHDTKHIEEDTKEKDEGFTNTLPIGLKLKIVERNIAKLSVLKHSLSLKVKYNRGQYEEDNTYRKVFKKLNISHNQEEFHKSMFSPLLRYYASLYKLINDFEIYTLSHYITDKYSTLNKVIASEQFHYRYGWNREEKKGELVKTDNYTFSTLLSKKYGHKNSQEISEMRNKISHFDEKILFKFPLEEVSSVPKGKGKYKKDEPIKSLKEKREEIVSLMEKQTDMQKVLGYDAINDFRMKTVQFQTKLKVYSNKEETIKKMIVEAKTPNDYYNIYKVKGVEGINEHLLNVIGETEAEKSIQEQIAEGNKVNV

SEQ ID NO: 105 - protein, 492 aa, synthetic construct
  REGION 1..492: note = CasM. 280852 Effector Protein
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 106 - protein, 477 aa, synthetic construct
  REGION 1..477: note = CasM. 292901 Effector Protein
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 107 - protein, 478 aa, synthetic construct
  REGION 1..478: note = CasM. 294601 Effector Protein
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 108 - protein, 477 aa, synthetic construct
  REGION 1..477: note = Effector Protein
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 109 - protein, 472 aa, synthetic construct
  REGION 1..472: note = CasM. 298142 Effector Protein
VPITKTISLRILRPYYPPEIEAKIKAEKEKRKENGDTGSLNSSYYRELKKEYPSIIINDEFFPLLSEMQRNITSIYNRTISHLYHRLIIKKESISTAKALSEGPYRDFKSTFNSYIALGLRQKVQSNFRKKDLMAFKIALPTAKSDKFPIPIYMQTNFKIKESPDSDFIIELPLVEYIAKETKGKNKMFTKVEILSPPKVKNIPVILSTRRRKESGQWFSDEGTNAEIRRIISGEYKVSWIEIVKRTRFGKHDWFVNMVISFEESQEGLDPDVIGGIDIGVSKPLICAINNSLDRYIVKGDDIIAFNRRALSRRRSLLRRNRLKRSGHGSRNKLEPITVLTEKNERFKKSIMQRWAKEVAEFFKSKRASIVQMEELTGIKEREDFFSKTLRMYWNYGQLQKTVENKLREYGIEVRYASPKDTSRRCHSCGHINDYFTFEFRQQNNFPLFKCMNCGIECSADYNAARNIAIAR

SEQ ID NO: 110 - protein, 500 aa, synthetic construct
  REGION 1..500: note = Effector Protein
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 111 - protein, 507 aa, synthetic construct
  REGION 1..507: note = Effector Protein
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 112 - protein, 529 aa, synthetic construct
  REGION 1..529: note = Cas14a.1 Effector Protein
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 113 - protein, 726 aa, synthetic construct
  REGION 1..726: note = Effector Protein
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 114 - protein, 517 aa, synthetic construct
  REGION 1..517: note = Effector Protein
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 115 - protein, 481 aa, synthetic construct
  REGION 1..481: note = Effector Protein
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 116 - protein, 358 aa, synthetic construct
  REGION 1..358: note = Effector Protein
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 117 - protein, 507 aa, synthetic construct
  REGION 1..507: note = Effector Protein
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 118 - protein, 534 aa, synthetic construct
  REGION 1..534: note = Effector Protein
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 119 - protein, 537 aa, synthetic construct
  REGION 1..537: note = Effector Protein
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 120 - protein, 540 aa, synthetic construct
  REGION 1..540: note = Effector Protein
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 121 - protein, 542 aa, synthetic construct
  REGION 1..542: note = Effector Protein
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 122 - protein, 564 aa, synthetic construct
  REGION 1..564: note = Effector Protein
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 123 - protein, 610 aa, synthetic construct
  REGION 1..610: note = Effector Protein
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 124 - protein, 369 aa, synthetic construct
  REGION 1..369: note = Effector Protein
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 125 - protein, 552 aa, synthetic construct
  REGION 1..552: note = Effector Protein
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 126 - protein, 534 aa, synthetic construct
  REGION 1..534: note = Effector Protein
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 127 - protein, 577 aa, synthetic construct
  REGION 1..577: note = Effector Protein
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 128 - protein, 613 aa, synthetic construct
  REGION 1..613: note = Effector Protein
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 129 - protein, 615 aa, synthetic construct
  REGION 1..615: note = Effector Protein
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 130 - protein, 775 aa, synthetic construct
  REGION 1..775: note = Effector Protein
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 131 - protein, 777 aa, synthetic construct
  REGION 1..777: note = Effector Protein
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 132 - protein, 610 aa, synthetic construct
  REGION 1..610: note = Effector Protein
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 133 - protein, 632 aa, synthetic construct
  REGION 1..632: note = Effector Protein
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 134 - protein, 625 aa, synthetic construct
  REGION 1..625: note = Effector Protein
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 135 - protein, 517 aa, synthetic construct
  REGION 1..517: note = Effector Protein
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 136 - protein, 410 aa, synthetic construct
  REGION 1..410: note = Effector Protein
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 137 - protein, 486 aa, synthetic construct
  REGION 1..486: note = Effector Protein
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 138 - protein, 602 aa, synthetic construct
  REGION 1..602: note = Effector Protein
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 139 - protein, 494 aa, synthetic construct
  REGION 1..494: note = Effector Protein
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 140 - protein, 526 aa, synthetic construct
  REGION 1..526: note = Effector Protein
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 141 - protein, 649 aa, synthetic construct
  REGION 1..649: note = Effector Protein
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 142 - protein, 414 aa, synthetic construct
  REGION 1..414: note = Effector Protein
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 143 - protein, 413 aa, synthetic construct
  REGION 1..413: note = Effector Protein
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 144 - protein, 449 aa, synthetic construct
  REGION 1..449: note = Effector Protein
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 145 - protein, 711 aa, synthetic construct
  REGION 1..711: note = Effector Protein
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 146 - protein, 574 aa, synthetic construct
  REGION 1..574: note = Effector Protein
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 147 - protein, 400 aa, synthetic construct
  REGION 1..400: note = Effector Protein
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 148 - protein, 743 aa, synthetic construct
  REGION 1..743: note = Effector Protein
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 149 - protein, 769 aa, synthetic construct
  REGION 1..769: note = Effector Protein
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 150 - protein, 666 aa, synthetic construct
  REGION 1..666: note = Effector Protein
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 151 - protein, 564 aa, synthetic construct
  REGION 1..564: note = Effector Protein
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 152 - protein, 565 aa, synthetic construct
  REGION 1..565: note = Effector Protein
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 153 - protein, 499 aa, synthetic construct
  REGION 1..499: note = Effector Protein
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 154 - protein, 560 aa, synthetic construct
  REGION 1..560: note = Effector Protein
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 155 - protein, 404 aa, synthetic construct
  REGION 1..404: note = Effector Protein
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 156 - protein, 392 aa, synthetic construct
  REGION 1..392: note = Effector Protein
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 157-201 - skipped (45 entries)

SEQ ID NO: 202 - RNA, 98 nt, synthetic construct
  misc_feature 1..98: note = Guide sgRNA Sequence
  source 1..98: mol_type = other RNA
tggggcagttggttgcccttagcctgaggcatttattgcactcgggaagtaccatttctcagaaatggtacatccaactctaggcgcccgctaagttc

SEQ ID NO: 203 - RNA, 98 nt, synthetic construct
  misc_feature 1..98: note = Guide sgRNA Sequence
  source 1..98: mol_type = other RNA
tggggcagttggttgcccttagcctgaggcatttattgcactcgggaagtaccatttctcagaaatggtacatccaaccccgggtaagcctgtctgct

SEQ ID NO: 204 - RNA, 98 nt, synthetic construct
  misc_feature 1..98: note = Guide sgRNA Sequence
  source 1..98: mol_type = other RNA
tggggcagttggttgcccttagcctgaggcatttattgcactcgggaagtaccatttctcagaaatggtacatccaaccgtgctgtttcctccccacg

SEQ ID NO: 205 - RNA, 98 nt, synthetic construct
  misc_feature 1..98: note = Guide sgRNA Sequence
  source 1..98: mol_type = other RNA
tggggcagttggttgcccttagcctgaggcatttattgcactcgggaagtaccatttctcagaaatggtacatccaacgtgccttagtttcttcatct

SEQ ID NO: 206 - RNA, 98 nt, synthetic construct
  misc_feature 1..98: note = Guide sgRNA Sequence
  source 1..98: mol_type = other RNA
tggggcagttggttgcccttagcctgaggcatttattgcactcgggaagtaccatttctcagaaatggtacatccaacgggggcgggggggagaaaaa

SEQ ID NO: 207 - RNA, 98 nt, synthetic construct
  misc_feature 1..98: note = Guide sgRNA Sequence
  source 1..98: mol_type = other RNA
tggggcagttggttgcccttagcctgaggcatttattgcactcgggaagtaccatttctcagaaatggtacatccaacgcgccctccgatctggggtg

SEQ ID NO: 208 - DNA, 98 bp, synthetic construct
  misc_feature 1..98: note = Guide nucleic acid
  source 1..98: mol_type = other DNA
tggggcagttggttgcccttagcctgaggcatttattgcactcgggaagtaccatttctcagaaatggtacatccaactctaggcgcccgctaagttc

SEQ ID NO: 209 - DNA, 98 bp, synthetic construct
  misc_feature 1..98: note = Guide nucleic acid
  source 1..98: mol_type = other DNA
tggggcagttggttgcccttagcctgaggcatttattgcactcgggaagtaccatttctcagaaatggtacatccaaccccgggtaagcctgtctgct

SEQ ID NO: 210 - DNA, 98 bp, synthetic construct
  misc_feature 1..98: note = Guide nucleic acid
  source 1..98: mol_type = other DNA
tggggcagttggttgcccttagcctgaggcatttattgcactcgggaagtaccatttctcagaaatggtacatccaaccgtgctgtttcctccccacg

SEQ ID NO: 211 - DNA, 98 bp, synthetic construct
  misc_feature 1..98: note = Guide nucleic acid
  source 1..98: mol_type = other DNA
tggggcagttggttgcccttagcctgaggcatttattgcactcgggaagtaccatttctcagaaatggtacatccaacgtgccttagtttcttcatct

SEQ ID NO: 212 - DNA, 98 bp, synthetic construct
  misc_feature 1..98: note = Guide nucleic acid
  source 1..98: mol_type = other DNA
tggggcagttggttgcccttagcctgaggcatttattgcactcgggaagtaccatttctcagaaatggtacatccaacgggggcgggggggagaaaaa

SEQ ID NO: 213 - DNA, 98 bp, synthetic construct
  misc_feature 1..98: note = Guide nucleic acid
  source 1..98: mol_type = other DNA
tggggcagttggttgcccttagcctgaggcatttattgcactcgggaagtaccatttctcagaaatggtacatccaacgcgccctccgatctggggtg

SEQ ID NO: 214 - RNA, 78 nt, synthetic construct
  misc_feature 1..78: note = Handle Sequence
  source 1..78: mol_type = other RNA
tggggcagttggttgcccttagcctgaggcatttattgcactcgggaagtaccatttctcagaaatggtacatccaac

SEQ ID NO: 215 - RNA, 13 nt, synthetic construct
  misc_feature 1..13: note = Repeat Sequence
  source 1..13: mol_type = other RNA
tggtacatccaac

SEQ ID NO: 216-217 - skipped (2 entries)

SEQ ID NO: 218 - protein, 8 aa, synthetic construct
  REGION 1..8: note = Nuclear localization signal Sequence
  SITE 3: note = misc_feature - Xaa can be any naturally occurring amino acid
  SITE 4: note = MISC_FEATURE - Xaa = W or F or Y
  REGION 5..6: note = misc_feature - Xaa can be any naturally occurring amino acid
KRXXXXAF

SEQ ID NO: 219 - skipped

SEQ ID NO: 220 - protein, 6 aa, synthetic construct
  REGION 1..6: note = Nuclear localization signal Sequence
  SITE 5: note = MISC_FEATURE - Xaa = K or R
  SITE 6: note = MISC_FEATURE - Xaa = W or F or Y
LGKRXX

SEQ ID NO: 221 - skipped

SEQ ID NO: 222 - protein, 16 aa, synthetic construct
  REGION 1..16: note = Nuclear localization signal Sequence
APKKKRKVGIHGVPAA

SEQ ID NO: 223 - protein, 18 aa, synthetic construct
  REGION 1..18: note = Nuclear localization signal Sequence
KRPAATKKAGQAKKKKEF

SEQ ID NO: 224 - RNA, 20 nt, synthetic construct
  misc_feature 1..20: note = Spacer Sequence
  source 1..20: mol_type = other RNA
tctaggcgcccgctaagttc

SEQ ID NO: 225 - RNA, 20 nt, synthetic construct
  misc_feature 1..20: note = Spacer Sequence
  source 1..20: mol_type = other RNA
cccgggtaagcctgtctgct

SEQ ID NO: 226 - RNA, 20 nt, synthetic construct
  misc_feature 1..20: note = Spacer Sequence
  source 1..20: mol_type = other RNA
cgtgctgtttcctccccacg

SEQ ID NO: 227 - RNA, 20 nt, synthetic construct
  misc_feature 1..20: note = Spacer Sequence
  source 1..20: mol_type = other RNA
gtgccttagtttcttcatct

SEQ ID NO: 228 - RNA, 20 nt, synthetic construct
  misc_feature 1..20: note = Spacer Sequence
  source 1..20: mol_type = other RNA
gggggcgggggggagaaaaa

SEQ ID NO: 229 - RNA, 20 nt, synthetic construct
  misc_feature 1..20: note = Spacer Sequence
  source 1..20: mol_type = other RNA
gcgccctccgatctggggtg

SEQ ID NO: 230 - protein, 4 aa, synthetic construct
  REGION 1..4: note = Linker Sequence
GAAA

SEQ ID NO: 231 - RNA, 61 nt, synthetic construct
  misc_feature 1..61: note = Handle Sequence
  source 1..61: mol_type = other RNA
tggggcagttggttgcccttagcctgaggcatttattgcactcgggaagtaccatttctca

SEQ ID NO: 232 - RNA, 65 nt, synthetic construct
  misc_feature 1..65: note = Handle Sequence
  source 1..65: mol_type = other RNA
tggggcagttggttgcccttagcctgaggcatttattgcactcgggaagtaccatttctcagaaa

SEQ ID NO: 233 - RNA, 20 nt, synthetic construct
  misc_feature 1..20: note = Repeat Sequence
  source 1..20: mol_type = other RNA
attgctccttacgaggagac

SEQ ID NO: 234 - RNA, 17 nt, synthetic construct
  misc_feature 1..17: note = Spacer Sequence
  source 1..17: mol_type = other RNA
gagcaacggcggaaggt

SEQ ID NO: 235 - RNA, 39 nt, synthetic construct
  misc_feature 1..39: note = crRNA Sequence
  source 1..39: mol_type = other RNA
ggattgctccttacgaggagacgagcaacggcggaaggt

SEQ ID NO: 236 - protein, 1224 aa, synthetic construct
  REGION 1..1224: note = M08 Effector Protein
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 237 - protein, 1228 aa, synthetic construct
  REGION 1..1228: note = CasM21 Effector Protein
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 238 - protein, 1159 aa, synthetic construct
  REGION 1..1159: note = CasM26 Effector Protein
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 239 - protein, 1160 aa, synthetic construct
  REGION 1..1160: note = Cas12d.3 Effector Protein
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAV

SEQ ID NO: 240 - protein, 1281 aa, synthetic construct
  REGION 1..1281: note = CasM. 21526 (CasFire) Effector protein
MLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 241 - protein, 1345 aa, synthetic construct
  REGION 1..1345: note = CasM. 279793 Effector Protein
MTKQNKSVTQNTKRKNFGEFTNLYSLSKTLRFELKPVKATKTILEVERKDRENFKKDRKIAKNYQKLKGILNELHQEFIQDVMREFSFQEKEIKEFEERYLEALNFKEKDNYKKRTQLKNAYEKVAKKLAGKIATAFGKYNQEKYGVKFTKKNLTGENVFDILEGKYKGDKKILGIIHTFKFKPTKEEKKQGKEAVNFSTYLGGFNQNRENFYKGEMKAGQFATRTIENLIQFLKNKKLFIDKYKDNYQKIGFSQEQVEIFNLNYFNNLFLQEGLDVYNGILGAKKGEKNTENDGLNQKINLFKQKEKTRCKANGEKFNKSDYPIFKELYKQIGSIKKDNDVYVEIKSDEELVNVLQSLPEKTANTLREVQKFYENFFDKIFNDEFDLDKIYLPKSVGTHFSHLAFSDWSKLAFVFNKRWRNEKVKIKEGEDVNVQSRSLADIKKRMEEILEMDGGVSFGKTYCQKVGLEKEARTIEDVWSGFWKIIQYHINSQFIGGEKEVFDKEKKDDKTEKIQTIDDLQEEYLQATEMYRERMVESEEGLNDGEEKEIKTKLKNYLDRIKDIERIARYFDLRKHFDDIDEASKDGDFYFIYQELLQDISEAKINDHYNEIRNYLTKANVVDDKFKLNFNDGQTLSGWDLNKETEKFSLIFKRKVDGGVEYYLGIINKEKNKTIFDKKKHPEIFTENSEFEKMEYKLFPSPSKMLPKIAFTKNKEGERIKPVFLDENAGKEIAQIKKEFALFQDAKKEDKNKWSDEFDRKKLNKLIDYYKLVLEKHPEKYMQTFNFVFKSSAKYKNLGEFNDDVARQNYVTKFVSVDKDYIDQKVESGELYLFKIHNKDWNLTKAGDTKKQSKKNLHTIYFEELFSEKNIAEPVFKLSGGAEVFFRDAIEKKKQKKKKDKKGKEILEKFRFTKNKILFHVPITINYGKPSINQGQFNQKINEFIADNSRSVNILGIDRGEKHLLYYSLVDNNGKIIKSGSLNEINGVDYHEKLDKAEKERQEARKSWQKINQIKNLKAGYISQVIKKIVDLAIENNAIIVLEDLNFGFKSFRQKIEKNVYQQFEKALIDKLGFVTDKEKLNHRQAPQLSAPFESFEKMGKQTGIVFYVLATNTSKVCPQCQWKKNIFPHYSTKKSIAENLQKQYKMKMYWRENENRFEFEYKGDGDKEFSSIFSNVDRVRYDKRANNNQGGYVIYQIDSTTKEKDGRNIKEKSITNLLKELLLEKFEIDNLEGELLVKLSEKSPDVSKETIKDFFGLLNSILNIRNSMTDTEEDYIQCPACGFDTRKENKIGIKNGDDNGAYNIALRGRFLIERIKKAKKEDKKPNLTFSNNDYFQWVREFVK

SEQ ID NO: 242 - protein, 1332 aa, synthetic construct
  REGION 1..1332: note = CasM. 286498 Effector Protein
MKSIFDNFTGLYSLSKTLRFELRPVGQTLENIKNGHFLESDKKMADDYQDVKKIIDNYHKFFIDDVLKGASFDWALLEKELTDFNKNKTDDSKVEAEQKKLREQIAKTLAGDKRFKSLTASTPNDLFNKDKDFIGWLEQSSVKEIRKDALDTFKKFSSYFKGFQENRKNVYSADDIPTAVPYRIVNDNFPKFLQNISIFKTIQEKCPQVIADVENELASYLGKEKLADIFTVQAFNKYLCQGGKENQRGIDFYNQVIGGIAEKEGGVNLRGINQFLNLYWQQHPDFAKENRRIKMVPLYKQILSDRSSLSFKIETIDTDEELKTAISEYADKLESKSNDEKKSVLDVCVELFDSIKEQNLQEIYVNRKDINNISRILTGDWSWLQSRMNLYADEVFTTKAEKTRWQKSVDGDEGENKSKGAYSLAELNRVLEYASENVAETDIRITDYFCHRNRFYYEKESGLFKQGEELVALSIKESCEDILSKRKAMNEAFANISESNSLRDNSEDIAKTKTYLDSVQDLLHRIKPLKVNGLGDPSFYAVFDSIYSALSEVISIYNKTRNYITRKAESPEKYKLNFDNPTLANGWDLNKEKDNTCVLLRKNGMYYLGIMNPKDKPKFAEKYDCGTESCYEKMIYKLLPGPNKMLPKVFFSTKGKKQYNPPENILHGYEQGKHKKGVAFDINFCHELIDWFKSAINQHEDWKKFGFKFSDTKSYKDISDFYREVTEQGYKLTFINIPESEISKMVSEGKLYLFQIYNMDFAPGANGMPNMHTLYWKNLFSEENLKDVVLKLNGEAELFYRPAGIKEPVVHAKGSYLVNRITKDGEPIPEKIHDEIYRNANGNLNNLSKEASEYKESHKVVIKQASHEIIKDRHYTEPKFLFHIPLTINFKAPSMKPTVCTAIQNENVRRFLKNNPDVNIIGLDRGERNLIYLSLINQKGEIIKQFSFNDVEREQNGQTVKVNYHEKLDQREKERDAARKSWQAIGKIAELKEGYLSAVIHQLTKLMVEYNAIVVMEDLNFGFKRGRFHVEKQVYQKFEHMLIDKLNYLVFKDRGLNEPGGALNGYQLAGQFESFQKLGKQSGMLFYVPAAYTSKIDPKTGFVSMMNFKDLTNVHKKRDFFSKFEDIHFDEATCSFVFTFDYKNFNGKAKEEMKQTKWAVYSREKRIVYFSKTKSSEDIMPTEKLRALFESDGIEYKSGNNIHDSVMAVGADLKEGAKPSKEIADFWDGLLYNFKLILQMRNSNAKTGEDYIISPVMASDGTFFDSRVEAKKGKDAKLPLDADANGAYHIALKGLSLINKINLAGEDELKKFDMKISNEDWFNFAQEKKYAE

SEQ ID NO: 243 - protein, 1265 aa, synthetic construct
  REGION 1..1265: note = CasM. 1895868 Effector Protein
MRTTTSLDAFTNRYALSKTLRFELKPIGNTQMMLEQNNVFAKDRAIREKYEKTKPWIDLLHREFVAESLQNAQLGNLDDYYAALQNVQKITKDTNAEDKKRWKKGFEKQEKRLRKEVVALFDKAAHIWATQRYPQLKKKTKDFLFEEGVFEHVLFARYGSAPDTTVKIVTSNPETGEVIDEREESIFKGWKGFTGYFDKFFETRKNFYKDNGTATAIATRAINQNLRRFAENMQKLTDIKNNYPELLAHTDFGDFDIAHAQSLDFYARTCLLQEGIDAYNKKFVGVLKSAINEYQQKNKGVRISYPKTLDNQILGERERRLFDVIEDDRELHDVFRAFVDDGTVFAAEMRQLAQAFSAQNGTYDYTQIYISKKGFETISRKYTHDTRAWHDALADVFKAKAKKRIATTASGEKKFPAYIPVAYITQALTLVQESEDTECTWKERYASITENKTLEEGFFAIFADEFERLFVHMEATVQDTDYVVAEDKAKKLLSDGQITKNEQTTQIIKEYADALLRIYQMAKYFAVEKKSMWDDAVAIDDTFYETFKEIYGNTHSTIVASYNLLRNYLTKKPWEDVQKWKLNFENPTLLDGWDKNKEAANFGVILRDGDKFYLGIMRKGHNNIFANQHHSNFEGQGLQKMVYKFFPDPKKMFPKVCFSAKGMEFFAPSEEIVRIYKNAEFKSGDTFNVESMQKLIDFYKNALQKYDGWKIYDFKHLKDTAQYTSNIGEFYDDVAKGGYQLGWQNISKEYVEEKNANGELYLFQIKNKDWNDGATGRKNLHTLYFEYLFSEKNAAADFVFRLNGGAEVFYRPAAIESKTERRGNREVAAKKRYTQDKVFLHVPITLNRTAGDVKTSAFNDAVNRFLAGNPDINIMGIDRGEKHLAYYSIIDQNGNRIVSGSFNTIGSKDYHALLTERQGAREEARKNWQRVEQIKDLKKGYISLVVREIADLAIKHNAIIVLENLNMRFKQIRGGIEKSVYQQLEKALIEKLNFLVNKGEVDATKAGHLLRAYQLAAPFETFEKMGNQTGIIFYTTASYTSQVDPVTGWRPHVYLKYRNAQKTKEDILRIFDDIVFNDEKQRFEFAYRHNGVSWTVCSSVERHRWNRSNNAGKGGYDVFPVEGEGSITQRLQEACAQRGIDTTRNILAQIDELDESASATVSFLRDLCFYFRLICQIRNTDDGADDINAQDFLMSPVEPFFDTRNAQEQYPQNGDENGAYNIARKGIIILQKITAWGRSQDTQRRYPDTFVSQDEWDTFLTQHTT

SEQ ID NO: 244 - protein, 1281 aa, synthetic construct
  REGION 1..1281: note = CasM. 291253 Effector Protein
MIKSYDDFTKLYSLQKTLRFELKPIGKTLEHIKKSEIIESDETLEEQYAIVKNIIDRFHRKHIDEALSLVDFTKHLDTFKTIQELYLKRGKTDREKKELEELSADLRKLIVSYLKGNVKQKTQHNLNPIKERFEILFGKELFTNEEFFTLAENKEEKKAIQAFKGFTTYFKGFQENRKNMYSEEDKSTAIAYRIINENMPLFIENIARFQKVLDVIEKTKLTELKQNLKTELKGHSVSDIFRIEYFNNVLTQEGISRYNTIIGGKTTTEGVKIQGLNEIINLHNQQSKDVKLPILKPLHKQILSEEYSTSFKIKAFENDNEVLKAIDTFWNEHIEKSIHPVTGKRFNILLKIENLCKKLEKYKDKEIEKLFIERKNLSTVSHQVYGQWNIIRDALRMHLEMNNKNIKEKDIDKYLDNDAFAWKEIKDSIKIYKEHVEDAKELDENGIVKYFSSMSINEEDDEKEYSISLIKNINEKYNNVKSILEEDRTGKSDLHQDKEKVAIIKEFLDSLKQLQWFLKLLYVTVPLDEKDYEFYNELEVYYEALLPLNSLYNKVRNYMTRKPYSVEKFKLNFYSPTLLDGWDKNKETANLSIILKKNGKYYLGIMNKENNTIFENFPKSKSNDYYEKMIYKLLPGPNKMLPKVFFSKKGLEYYKPSKEILRIYEKGEFKKDKSGNFKKESLHTLIDFYKEAIAKNEDWKIFKFKFKNTREYEDISQFYRDVEEQGYLIIFEKVDANYVDKLVEEGELYLFQIYNKDFSENKKSKGNPNLHTIYWESLFDNQNLKDVVYKLNGEAEVFYRKKSIDYPEEIYNNGHHKEELNGKFNYPIIKDRRYTQDKFLFHVPITMNFISKEEKRVNQLACEYLSTTKEDVHIIGIDRGERHLLYLSLIDKEGNIKKQLSLNTIKNENYDKEIDYRVKLDEKEKKRDEARKNWDVIENIKELKEGYMSQVIHIIAKMMAEEKAILIMEDLNIGFKRGRFKVEKQVYQKFEKMMIDKLNYLVFKNKEPLEPGGSLNAYQLTSKFDSFKKLGKQSGFIFYVPSAYTSKIDPTTGFYNFIRVDVPNLEKGKEFFSKFEKIIYNTKEDYFEFHCKYGKFVPEPKNKDNDRKTKESLTYYNAIKDTVWVVCSTHHERYKIVRNKAGYYESQPVDVTKNLRDIFSEANINYSDGKDIKPIIIESNNAKLLKSIAEQLKLILAMRYNNGKHDDDEKDYILSPVKNKQGKFFCSLDGDQSLPINADANGAYNIALKGLLLIEKIKKQQGKAKDLYISNLEWFMFMMSR

SEQ ID NO: 245 - protein, 1259 aa, synthetic construct
  REGION 1..1259: note = CasM. 1895869 Effector Protein
MLKEKQFKTFGDFTNLYELSKTLRFELRPTPETKDLLDKNKIIQTDKKIAENYQEIKKYFDKLHKKFIKEALSNTQIDFSDFCKLWEQNSKDSGKIKDLSRKLRKSIKQAFDKKGADWHKRYLEKGIKLKKKNLDILFEERVLDILKEEFKDDVDVKLFESFKGFSTYFTNFHESRKNFYKDDGTASAIATRIIDENLKRFCDNIKVKKHSKKLISELNEREAKIFEADFYNRCLLQQGIDDYNQVIGDINKKINNLRQNKIENTPTLKILYKQILGDVRRQETEQDAFIEIKNNEEVFDFLQDFIKHSDENNKYFKNLFYKFIEGKHSLDKIFLAKRFVNTISGKWFASWEVFGAELIKKFGNKKDLPDFIPFAAVKDVLQNCNIPANELFKEKIKNDEDKNIYDIFINLWKEEFDSNLKKVEESKKEVENMIAEDKVYSNKKEKRKNDNGEEIEIEIQKEKIKNYADAAMNIFRMMKYFLLEKNGKTVEGMGEDNNFYNELNIVFKGGEIDGKVYEGVKTYLYYNEFRNYLTKKPFNEEKTKLNFDCGQILSGWDKNKESEKLGVILRKDNKYYLAIINKKHNKIFDVKKNSYAYIVGDNFYEKMEYKLFPDAKRMIPKIAFAKNNKEKFGWTDEIQKIKNEYAEFQEGKKNDKNLWKDKFNKNKMEKLITYYQNCLEKGGYKDIYNFRWKSPDKYSGIGEFNDEIDRQSYCLKFVKVDFNYVFEKVKSGELYLFQIYNKDFSDKADRAQKENIHTEYFKLLFDQRNLDNVVLKLSGGAEIFYRPKTEGLPKKKDNKGNEVVRHRRYADDKYFLHLPIQLNFGRGNLSGGEFNSKINQYLSEQREIKIIGIDRGEKHLAYYSVINQDGKIEEIESLNTVNGIDYRKKLDELEKKREQERKSWQSISKIKDLKKGYISHVIKKICDLAIEHNAIIVFEDLSGGFKNSRKKIEKQIYQNLELALATKLNYLTFKDKNFGESGHYLNAYQLAPKIDNYQDIKMQTGIVFYTPAGYTSSTCPQCGFRKTLKFDYTATISKAEDLIRGSKLNIVFEKEKNRFKINYLFNPIEKKKKKIKENELFADAGAKNEFTIYSDVKRIKWHNTGTKRLEEAEGERLLENKNSRGRDKEYDINKCLTRLFRENKIDVNGDIIGQITKIKSLKLYQDLFYYLFLATLIRNNVSGSDIDYIQCPSCHFHSDGGFQKQKFNGDANGAYNIARKGILILKKIKQFAAQDKDMKNFGWKHLTVDINEWDKFTQK

SEQ ID NO: 246 - protein, 1371 aa, synthetic construct
  REGION 1..1371: note = CasM. 1895866 Effector Protein
MNKNFSNFTELYTLSKTLRFELKPVAQTKENIKKGKFLESDKKKADDYKDVKKIIDNYHKFFIDDVLKNASFDWTVLEKEMSDFNKSKADDSKVEAEQKKLRDQIAKKLTSDKRFKALTASTPSDLFNKDKDFIDWFTQNSTKDINKEALETFKRFSSYFKGFQENRKNVYSAEPIPTAVPYRLVNDNFPKFLQNIALFKIIQEKCPQVISDVEKELASYLGKEKLADIFTVQAFNKYLCQGGKENQRGIDFYNNILGGIAEKEGGINLRGINQFLNLYWQQHQDFAKQNKRIKMIPLFKQILSDRSSLSFKIESINTDQELLTSITEYADKLETKSNDEKKSVLDICSDLFASIKAQNLQEIYVNRKDINSISRILTGDWSWLQSRMNVYADEVFTTKAEKTRWQKSIDGDEGENKSKGVFSLAELNSVLEYSSENVSETDVRITDFFDHRNRFYYEKESGLFKQGDELVALSIRESCEDILAKRKAMDEAFANVSENNSLRDNSEDVAKIKIYLDCVQELLHRIKPLKVNGLGDPAFYAVFDTVYNSLSEVISLYNKTRNYITKKAANPEKYKLNFDNPTLADGWDLNKEQANTSVLMRKDGMYYLGIMNPKDKPKFAEKYECGNEACYEKMIYKQFDATKQIPKCSTQVKEVKKHFQSGATDSIILNDKSKFKLDLVITKEIWFLNNHVWNGEKFVPKRESNETRPKKFQIGYYKQTGDLGGYKEALNIWISFCKTFLQSYISSSIYDYDFKESSNYDSLDEFYNYLNATCYKLSFINIPEATISQMVSEGKLYLFQIYNKDFAPGASGMPNMHTLYWKNLFSEENLKNVVLKLNGEAELFYRPAGIKEPVIHAKGSYLVNRITKDGEPIPEKIHDEIYRNANGKLESLSKEATEYKASHKVVIKEAKHDIIKDRHYTEPKFLFHVPLTINFKASGNSYINENVRRFLKNNPDVNVIGLDRGERHLIYLSLINQKGEIIKQFTFNEVERNKNGQVIKVNYHEKLDQREKVRGAARKSWQAIGKIAELKEGYLSAVIHQLTKLMVEYNAIVVMEDLNFGFKRGRFHVEKQVYQKFEHMLIDKLNYLVFKDRGLTEAGGVLNGYQLAGQFESFQKLGKQSGMLFYVPAGYTSKIDPKTGFASMSNFKDLTNVHKKRAFFSKFDDIHFDDATGSFVFTFDYKNFDGKAKEEMKRTKWSVYSKDKRIVYLSKTKSYEDVQPTEKIKASLESVGIEYMSGNNLIDSIMVIGAELKDGAKPSKEIADFWDRLLYNFKLILQMRNSNAKTGEDYIISPVMADDGTFFDSREEFKKGENAKMPVDADANGAYHIALKGLSLLKRFDAASENELKKFDMKISNVDWFKFAQEKSYAE

SEQ ID NO: 247 - RNA, 20 nt, synthetic construct
  misc_feature 1..20: note = Spacer Sequence
  source 1..20: mol_type = other RNA
gtgaggaagcacctgagccc

SEQ ID NO: 248 - RNA, 20 nt, synthetic construct
  misc_feature 1..20: note = Spacer Sequence
  source 1..20: mol_type = other RNA
ggttctgggaggaaaagtcc

SEQ ID NO: 249 - RNA, 20 nt, synthetic construct
  misc_feature 1..20: note = Spacer Sequence
  source 1..20: mol_type = other RNA
gtgagagtatggagatgcca

SEQ ID NO: 250 - RNA, 20 nt, synthetic construct
  misc_feature 1..20: note = Spacer Sequence
  source 1..20: mol_type = other RNA
agcctttcaaagccaagtcc

SEQ ID NO: 251 - RNA, 20 nt, synthetic construct
  misc_feature 1..20: note = Spacer Sequence
  source 1..20: mol_type = other RNA
atggtgaacttctcctctgc

SEQ ID NO: 252 - RNA, 20 nt, synthetic construct
  misc_feature 1..20: note = Spacer Sequence
  source 1..20: mol_type = other RNA
ggacctaaagaaactggagt

SEQ ID NO: 253 - RNA, 20 nt, synthetic construct
  misc_feature 1..20: note = Spacer Sequence
  source 1..20: mol_type = other RNA
ctcgctgcatccctgctcag

SEQ ID NO: 254 - RNA, 20 nt, synthetic construct
  misc_feature 1..20: note = Spacer Sequence
  source 1..20: mol_type = other RNA
tgtgcttccggacatggtgt

SEQ ID NO: 255 - RNA, 20 nt, synthetic construct
  misc_feature 1..20: note = Spacer Sequence
  source 1..20: mol_type = other RNA
ctccatcagccactgacctg

SEQ ID NO: 256 - RNA, 20 nt, synthetic construct
  misc_feature 1..20: note = Spacer Sequence
  source 1..20: mol_type = other RNA
gcgagtttgtaggcacccag

SEQ ID NO: 257 - RNA, 20 nt, synthetic construct
  misc_feature 1..20: note = Spacer Sequence
  source 1..20: mol_type = other RNA
ccgaggccctgccttcgctc

SEQ ID NO: 258 - RNA, 20 nt, synthetic construct
  misc_feature 1..20: note = Spacer Sequence
  source 1..20: mol_type = other RNA
ctgcatccctgctcaggcta

SEQ ID NO: 259 - RNA, 20 nt, synthetic construct
  misc_feature 1..20: note = Spacer Sequence
  source 1..20: mol_type = other RNA
ctgttaagaagctccaggta

SEQ ID NO: 260 - RNA, 20 nt, synthetic construct
  misc_feature 1..20: note = Spacer Sequence
  source 1..20: mol_type = other RNA
cagttgatggtgtctgtgtc

SEQ ID NO: 261 - RNA, 20 nt, synthetic construct
  misc_feature 1..20: note = Spacer Sequence
  source 1..20: mol_type = other RNA
cagatgcagttattgtacaa

SEQ ID NO: 262 - RNA, 20 nt, synthetic construct
  misc_feature 1..20: note = Spacer Sequence
  source 1..20: mol_type = other RNA
gctcccacgtcgcagatgca

SEQ ID NO: 263 - RNA, 20 nt, synthetic construct
  misc_feature 1..20: note = Spacer Sequence
  source 1..20: mol_type = other RNA
ctgccagccttcggaggtgt

SEQ ID NO: 264 - RNA, 20 nt, synthetic construct
  misc_feature 1..20: note = Spacer Sequence
  source 1..20: mol_type = other RNA
gaagacacagctggggagct

SEQ ID NO: 265 - RNA, 20 nt, synthetic construct
  misc_feature 1..20: note = Spacer Sequence
  source 1..20: mol_type = other RNA
tcctggagagaacaggcaat

SEQ ID NO: 266 - RNA, 20 nt, synthetic construct
  misc_feature 1..20: note = Spacer Sequence
  source 1..20: mol_type = other RNA
gggacgagggtgtctcgcag

SEQ ID NO: 267 - RNA, 20 nt, synthetic construct
  misc_feature 1..20: note = Spacer Sequence
  source 1..20: mol_type = other RNA
ttgagctgcctgaatctccc

SEQ ID NO: 268 - RNA, 20 nt, synthetic construct
  misc_feature 1..20: note = Spacer Sequence
  source 1..20: mol_type = other RNA
ctcaaggcagccctgtggag

SEQ ID NO: 269 - RNA, 20 nt, synthetic construct
  misc_feature 1..20: note = Spacer Sequence
  source 1..20: mol_type = other RNA
gaggtgtcctcatgtggaga

SEQ ID NO: 270 - RNA, 20 nt, synthetic construct
  misc_feature 1..20: note = Spacer Sequence
  source 1..20: mol_type = other RNA
tccccgatcttgttctcact

SEQ ID NO: 271 - RNA, 20 nt, synthetic construct
  misc_feature 1..20: note = Spacer Sequence
  source 1..20: mol_type = other RNA
ggaggtcagggcaggttcag

SEQ ID NO: 272 - RNA, 20 nt, synthetic construct
  misc_feature 1..20: note = Spacer Sequence
  source 1..20: mol_type = other RNA
cagctctcagccaccttccc

SEQ ID NO: 273 - RNA, 20 nt, synthetic construct
  misc_feature 1..20: note = Spacer Sequence
  source 1..20: mol_type = other RNA
accagcttgacatcacagga

SEQ ID NO: 274 - RNA, 20 nt, synthetic construct
  misc_feature 1..20: note = Spacer Sequence
  source 1..20: mol_type = other RNA
agaaaagctttgaaacaggt

SEQ ID NO: 275 - RNA, 20 nt, synthetic construct
  misc_feature 1..20: note = Spacer Sequence
  source 1..20: mol_type = other RNA
gaacccaatcactgacaggt

SEQ ID NO: 276 - RNA, 20 nt, synthetic construct
  misc_feature 1..20: note = Spacer Sequence
  source 1..20: mol_type = other RNA
tatctgtaaaaccaagaggc

SEQ ID NO: 277 - RNA, 20 nt, synthetic construct
  misc_feature 1..20: note = Spacer Sequence
  source 1..20: mol_type = other RNA
gtgaataggcagacagactt

SEQ ID NO: 278 - RNA, 20 nt, synthetic construct
  misc_feature 1..20: note = Spacer Sequence
  source 1..20: mol_type = other RNA
cgctactctctctttctggc

SEQ ID NO: 279 - RNA, 20 nt, synthetic construct
  misc_feature 1..20: note = Spacer Sequence
  source 1..20: mol_type = other RNA
gatggatgaaacccagacac

SEQ ID NO: 280 - RNA, 20 nt, synthetic construct
  misc_feature 1..20: note = Spacer Sequence
  source 1..20: mol_type = other RNA
agacatgtaagcagcatcat

SEQ ID NO: 281 - RNA, 20 nt, synthetic construct
  misc_feature 1..20: note = Spacer Sequence
  source 1..20: mol_type = other RNA
ggccgagatgtctcgctccg

SEQ ID NO: 282 - RNA, 20 nt, synthetic construct
  misc_feature 1..20: note = Spacer Sequence
  source 1..20: mol_type = other RNA
ctccgtggccttagctgtgc

SEQ ID NO: 283 - RNA, 20 nt, synthetic construct
  misc_feature 1..20: note = Spacer Sequence
  source 1..20: mol_type = other RNA
gcccgaatgctgtcagcttc

SEQ ID NO: 284 - RNA, 20 nt, synthetic construct
  misc_feature 1..20: note = Spacer Sequence
  source 1..20: mol_type = other RNA
atctatgaaaaagacagtgg

SEQ ID NO: 285-328 - skipped (44 entries)

SEQ ID NO: 329 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgtgaggaagcacctgagccc

SEQ ID NO: 330 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacggttctgggaggaaaagtcc

SEQ ID NO: 331 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgtgagagtatggagatgcca

SEQ ID NO: 332 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacagcctttcaaagccaagtcc

SEQ ID NO: 333 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacatggtgaacttctcctctgc

SEQ ID NO: 334 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacggacctaaagaaactggagt

SEQ ID NO: 335 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacctcgctgcatccctgctcag

SEQ ID NO: 336 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaactgtgcttccggacatggtgt

SEQ ID NO: 337 - RNA, 98 nt, synthetic construct; identical to SEQ ID NO: 336
  misc_feature 1..98: note = Guide sgRNA
  source 1..98: mol_type = other RNA

SEQ ID NO: 338 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgcgagtttgtaggcacccag

SEQ ID NO: 339 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacccgaggccctgccttcgctc

SEQ ID NO: 340 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacctgcatccctgctcaggcta

SEQ ID NO: 341 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacctgttaagaagctccaggta

SEQ ID NO: 342 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaaccagttgatggtgtctgtgtc

SEQ ID NO: 343 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaaccagatgcagttattgtacaa

SEQ ID NO: 344 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgctcccacgtcgcagatgca

SEQ ID NO: 345 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacctgccagccttcggaggtgt

SEQ ID NO: 346 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgaagacacagctggggagct

SEQ ID NO: 347 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaactcctggagagaacaggcaat

SEQ ID NO: 348 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgggacgagggtgtctcgcag

SEQ ID NO: 349 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacttgagctgcctgaatctccc

SEQ ID NO: 350 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacctcaaggcagccctgtggag

SEQ ID NO: 351 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgaggtgtcctcatgtggaga

SEQ ID NO: 352 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaactccccgatcttgttctcact

SEQ ID NO: 353 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacggaggtcagggcaggttcag

SEQ ID NO: 354 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaaccagctctcagccaccttccc

SEQ ID NO: 355 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacaccagcttgacatcacagga

SEQ ID NO: 356 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacagaaaagctttgaaacaggt

SEQ ID NO: 357 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgaacccaatcactgacaggt

SEQ ID NO: 358 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaactatctgtaaaaccaagaggc

SEQ ID NO: 359 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgtgaataggcagacagactt

SEQ ID NO: 360 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaaccgctactctctctttctggc

SEQ ID NO: 361 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgatggatgaaacccagacac

SEQ ID NO: 362 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacagacatgtaagcagcatcat

SEQ ID NO: 363 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacggccgagatgtctcgctccg

SEQ ID NO: 364 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacctccgtggccttagctgtgc

SEQ ID NO: 365 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacgcccgaatgctgtcagcttc

SEQ ID NO: 366 - RNA, 98 nt, synthetic construct
  misc_feature 1..98: note = Guide sgRNA
  source 1..98: mol_type = other RNA
tggggcagttggttgcccttagcctgaggcatttattgcactcgggaagtaccatttctcagaaatggtacatccaacatctatgaaaaagacagtgg

SEQ ID NO: 367 - RNA, 78 nt, synthetic construct
  misc_feature 1..78: note = Guide sgRNA
  source 1..78: mol_type = other RNA
tggggcagttggttgcccttagcctgaggcatttattgcactcgggaatcaatccaacctccgtggccttagctgtgc

SEQ ID NO: 368 - RNA, 51 nt, synthetic construct
  misc_feature 1..51: note = Handle Sequence
  source 1..51: mol_type = other RNA
tggggcagttggttgcccttagcctgaggcatttattgcactcgggaatca

SEQ ID NO: 369-404 - skipped (36 entries)

SEQ ID NO: 405 - protein, 483 aa, synthetic construct
  REGION 1..483: note = CasM. 19498 Effector Protein
LHETEKSLKFAEKYIAMPTITRKIELTLCTEGLSDEQRKEQWGLLYHINDNLYKAANNISSKLYLDEHVSSMVRMKHAEYLSLQKELARAEKQKVDDAIIVELTRKLAVAEKEMTDQELAICKYATEMSTNTLAYNFAKEIETKIFGQILACLENNAHALFVDDSPNVRRGERAIRNYKKGMPIPFPWNRSIKIEADGGEFYLRWYNGLRFLLTFGKDRSNNRLIVKRCMKMDEVFEGEYKLCNSSIQLAKRDGKPKLFLLLVVNIPQEHVELNKNIVVGVDLGVNVPAYVATNITEERKAIGDREHFLNTRMTFQKRYKSLQRLKGTAGGKGRTKKLEPLERLRDAERNWVHTQNHLFSREVVNFAVQARAATINIEDLSGFGKDNDGNADEKKEFVLRNWSYYELQNMITYKASKYGIKVEKIRPDYTSKTCSWCGQQGFREGVTFICENPECKQHGEKIHADYNAARNIANSKDIIKKNE

SEQ ID NO: 406 - protein, 467 aa, synthetic construct
  REGION 1..467: note = CasM. 19548 Effector Sequence
MAETKRLQKVAKFQIVKPVNMSWDELGRMLRDVRYRLSRLANMAVSETYQNLHQRYRLKNQDAPKSLKIGQLSRNLRKILREEGVEEENLSKYSKTCVLPDTITGAFSRYKLSSIDWRKVLTGKISVPNYKTNLSIPIRCDKPHQRRLELTETGEIEADLMICNKPYPRVLLSTRTISDGQRTVLERLVSNKTNFLPGYRHRFFEVKEKKGKWELSVTYDFPKAEATRLHPDIIVGVDLGWSVPLYAAINNGYARIGYRKFEPLAKRIKHLQKQIKGRRFSTQKGGVKDLAQPTARAGHGRKRILKPIEKLEYKIDNAYTTLNHQLSHCVVEFAKNNGAGLIQIENLEGLKDDLSGTFIGQNWRYNQLQNFIKYKADEAGIKVHPVNPCYTSRRCSHCGFIHISFDREYRDKNRKNGKATMFECPKGCKPLNADYNAAKNLATFDIEEKIRLQCKQQSIEYKELPKD

SEQ ID NO: 407 - protein, 472 aa, synthetic construct
  REGION 1..472: note = CasM. 19910 Effector Protein
MPGTEKRLQKVATFEIVKPVNMSWPEFGKMLRDVRYRYWRLANMAVCENYMRFYQWRTQQTDANDRYKVKTLNRILRKMLIEEKNADEKELSRYSRDGAVSGYICGAFEKTKLSAVKSSSKWKKVIAGKESLPLFKKDLAIPINCSDHQPRLIERTQSGEYEVDLRICQQPYPRVLLSTAKISDGQKAILERLVSNETNSLPGYRHRFFEIKEKRNKWYLSVSYDFPKIDATRLHPNIIVGVDLGWSVPLYAAISNGYARIGYRKLKALGDRIKALQRQTIARRRSIQRTGEQDLSAPTARSGHGRKRILHPIEKLEGKIDNAYKTLNHQLSHCVIEFAKNHGAGLIQVENLKGLAEELSGTFIGQNWRYNQLQEFIKYKAKEAGIEVKEVNPCYTSRRCSECGFIHKEFTFEYRQANKKTDKATMFECPKCGYKAIADYNAARNLANPDIAEKIRLQCKEQGIEYKELPKD

SEQ ID NO: 408 - protein, 468 aa, synthetic construct
  REGION 1..468: note = CasM. 19948 Effector Protein
MPTITRKIELHFCTEGLSDEKQKEQRQLLYHINDNLYKAANNISSKLYLDEHVSSMVRLKHADYLSLQRELARAEKQKTPDDELITELSRKLSAAEKEMTDQELAICKYATEMATSTLAYNFAKEMETEIFGQILACLENNAHAVFVDDSLSVKRGERAIRNYKKGMPIPFPWNKNIKIETKDCEFYLRWYNGIRFRLHFGKDRSNNRLIVQRCLKLDDNFESEYKLCNSSIQLDKRDGKTKLFLLLVVNIPQEHVELNKNIVVGVDLGLNYPAYVATNSTEERKYIGDRDHFLKIRMQFQSRYKSLQRLKGTAGGKGRAKKLEPLERLRKAERNWVHTQNHLFSRDVVNFAVQTHAATIHMEDLSGFGKDNDGNADEKKEFVLRNWSYYELQSMIEYKAAKYGIKVEKIRPAYTSKTCSWCGQQGDRKSTTFICENPECKHYGESIHADYNAARNIANSKDIVKKNE

SEQ ID NO: 409 - protein, 453 aa, synthetic construct
  REGION 1..453: note = CasM. 265291 Effector Protein
MSKITRKIEIIPDIDGITHEESNKKCYNTFYKFDRKLYKVANLLVSQLYGLDNLLSLMRLQNDEYVKCQSKLSFKSITDATKEEIKKRMQEIDAELVSMKNDIAPKHPQTYSYRAVTSSEYAKDIPSDILNNLKQDVYQHFNENKKEQIRGERSLATYKKGMPIPFSFEKRHVIICDGDNYYLPWFEDTRFRLNFGRDRSNNRAIIDNCIKTKKYKLCAAAKIQLKERKLFLLITVDIPKAESVPVKGKVMGVDLGVINPAYVAVNDGPERSRIGNGEAFQKQRDVFRRRFRELQRSQLTQGGHGRKHKTKATEILRGKERNWVQTENHRISREIVNLASRWKVETIQMESLKGFGKNQEGEVEYNHKRLLGRWSYFELQKDIEYKAAMAGIAVQYVNPAYTSQTCHVCGQRGNRIERDTFICTNPECTCYNQAQDADMNAAINIAKSKDVIK

SEQ ID NO: 410 - protein, 468 aa, synthetic construct
  REGION 1..468: note = CasM. 270012 Effector Protein
MPTITRKIELTLCTDGLSDEERKAQWGLLYHINDNLYKAANNISSKLYLDEHVSSMVRLKHAEYLSLQKELAKAERQKMPDVDVIEELRERLSAAEQEMSDQELAICKYATEMSTNTLAYRFATEIETNIFGQILARLENNAQAVFLTDAPDVKRGERAIRNYKKGMPIPFPWNNSIKIECEGGEFYLRWYSGLRFHFNFGKDRSGNRLIVQRCLKLDKEYDGEYKLCNSSIQMVKRDGSTKFFLLMVVNIPQEYVELNKHIVVGVDLGINVPAYVATNITPERKAIGDREHFLNTRMAFQRRYKSLQRLKTTAGGKGRTKKLEPLERLRQAEHNWVHTQNHLFSREVVNFALQTHAATIHLEDLSGFGKDSDGNADERKEFVLRNWSYYELQNMITYKAAKYGIRVEKIRPAFTSRTCSCCGHEGFREGVTFICENPECQQFGEKVHADYNAARNIANSKDIIKKNE

SEQ ID NO: 411 - protein, 468 aa, synthetic construct
  REGION 1..468: note = CasM. 272451 Effector Protein
MPTITRKIELTLCTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTETLAYKFATEIETNVFGQILACLKQAAQSNFKNDAKDVKRGERAIRNYKKGMPIPFPWNDSLRIEKDNKDFYLRWYNGLRFLFNFGKDRSNNRLIVERCLKMDADYDGEYKLCNSSIQIAKREGKVKLFLLLVVSIPQEHVELNKKVVVGVDLGINVPAYVATNITEERKAIGDREHFLNTRMAFQRRYKSLQRLKGTAGGKGRTKKLEPLERLRKAEHNWVHTQNHLFSREVVDFAVKTHAATIHMEDLSGFGKDNDGNADERKEFVLRNWSFYELQNMITYKAAKYGIKVEKIRPAYTSKTCSCCGRQGFRSGVTFICENPECKQYGEKVHADYNAARNIANSKEIIKKNE

SEQ ID NO: 412 - protein, 480 aa, synthetic construct
  REGION 1..480: note = CasM. 274429 Effector Protein
MKNNVEEKRPDKEKRLTKVATFQIVKPVNMSWSEFGKMLRDVRYRLSRLANMAVSEAYQNLHQRYRLKNQNAPKSVKIGQISRDLRKILLEEGLEEENLSKYSKMCVLPDTITGAFSRYKLSTIDWRKVLTGKISIPNYKANLSIPIRCDKPQQRRLERTETGEIEVDLMICNKPYPRVLLSTRTISDGQRSVLERLVLNNANSLPGYRHRIFEIKEKRNEWYLSVTYDFPKAETTKLHSDIIVGVDLGWSVPLYAAINNGYARIGYKQLKPLGDSIKALQRQTIARRRSIQRGGTQDLAAPTARSGHGIKRILQPIEKLEGKIDNAYKTLNHQLSHCVIEFAKNHGAGVIQIENLKGLAEELSGTFIGQNWRYYQLQEFIKYKAKEAGIIVKEVNPFYTSRRCSECGYIHKDFTFEYRQANRKNGKSTMFECPKKEEKGCKPLNADYNAARNLATSDIEDKIRLQCKEQGIEYKEIKEK

SEQ ID NO: 413 - protein, 442 aa, synthetic construct
  REGION 1..442: note = CasM. 277378 Effector Protein
VTLLVKVVKIHLISEQFDKAGNRIDYKEVNKILWELQKQTREAKNKTVQLLWEWNNFSSDYVKASGIYPKAKDIFGYSSVHGQANKELRTKLILNSSNLSTTTMDVCKIFNTYKKEVWEGKRSVPSYKSDQPLDLHNDSIKLIYENKEFYVRLGLLNRAGFAKYGFKDGFRFKMQVKDNSTKTILERCFDGIYTIVASKLLYDQKKNRWKLNLSYSFDNKNISELDKEKILGVDVGVSCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKADRFLKDWTYYDLQTKIENKAKEVGINVVYIAPKYTSQRCSKCGYIHKDNRPNQAKFRCLKCDFESNADYNASQNIGIKNIDKTIKKERKKQKSEAQVNEK

SEQ ID NO: 414 - skipped

SEQ ID NO: 415 - protein, 487 aa, synthetic construct
  REGION 1..487: note = CasM. 281050 Effector Protein
MPDTDKGKRLTKVATFQIVKPVNMSWNEFGKMLHDVRYRYWRLANMAVCENYMRFYRWRTQQTDTNDHYKVKIINGILRKMLIEEKNADEKELSRYSRDGAVSGYVYGAFTQTKLSAITSKSKWGEVIKGKSALPLFKRDTSIPIMCTDKKPSMIEKTASGEYEVDLRICLKDKQLRPNGYPSVLLSTTKISDGQKAVLERLVSNKTNSLPGYRHRFFEVKEKRGDWYLSVSYDFPQAEATRLHPDIIVGVDLGWSVPLYAAINNGYARIGWRKLEPLAKSIKHLQKQTIVRRRSFQKGGKKDLAASTARTGHGIKRILQPIEKLEGKIDNAYKTLNHQLSHCIIEFAKNHGAGVIQIENLKGLAEELSGTFIGQNWRYHQLQEFIKYKAEEAGIAVKEVNPRYTSRRCSKCGYIHIGFDREYRDKNRKNGKSTMFECPECSKRIKDYKPLNADYNAAKNLATADIEEKIRLQCKEQGIEYKELPKD

SEQ ID NO: 416 - protein, 471 aa, synthetic construct
  REGION 1..471: note = CasM. 285333 Effector Protein
MPTITRKIKLELCTKGLSEEERKAQWNLLYHINDNLYRSANNISSKLYLDEHVSSLVWLKHKEHQTLKADLAKAKKQKIQDEKTIAELESRLKSCESEMSDQELAICKYTDEMSSKTLSYKFATELELNIYAQILTQVQSKVYADFQNDQKDVRDGKRAIRTYKKGMPIPFPWRNNIRMEPVKKGREYEFYIKWYNDIRFQLIFGKDRSNNRLILQRCFKLDENCVEDYQMRTSSIKMVKGANGTELFLYLVVDIPQEKHILNNKIVVGVDLGINVPAYVATNVTDDRKAIGDREHFLNTRMAISKRFHSFQRLKGTTGGRGKTKKLEPLERLKEKERNWVHTQNHLFSRDVITFALHVKAATIQMEDLSGYGKDDEGNVVEEKKFLLGKWSYYELQEMIKYKAKKVGMRVNFIKPAYSSQTCSWCGERGERNSTSFVCTNSECSHYGEDLHADYNAARNIARSKNIIRYE

SEQ ID NO: 417 - protein, 377 aa, synthetic construct
  REGION 1..377: note = CasM. 286285 Effector Protein
MIITRKIQILFAAQGEEFKKDKDTLYKWSNIVHHASNIVASNKYVCDHLQGMVYLTEEGKEAVSELSQKVDDIFNTSRMNTTYRMISSLYKGEIPTDILSCVNMQVSKLYNKERKKMADGDRSLRSYRSNIPIPFSANSLMRKWKYADKEYSFDLFGIPFKVVLGKDKSNNRSILERLMDGTYKAATSSIKIQNCEDETGKKTRKFFLLLCVEIPDKSYAGREDNILFAELSIDHPLLVSFPIKKEESKPIPIGNKQSYLYKRLQIQKGLDSCKASCKWNKGGRGRKRKMKSTERFKAKEHNFVDAYMHQISAALIKFAIKHDIGKLCLVDVDKKIKEAKESPFVLRNWSYYSLLTKIQYKAKMNGITVVMVDKNVL

SEQ ID NO: 418 - protein, 483 aa, synthetic construct
  REGION 1..483: note = CasM. 286678 Effector Protein
MPTITRKIRLHLCTDGLSEEERKAQWKMLYRINDNLYRAANNISSKLYLDEHISSMVRLKHAEYTSLKTELLKAKKADDEETVAELEARINVLNAELSAQEEAICSYATEMATRTLAGKFASELDLNIYGQILAEVKSVVFKNFNSDSKDVREGKRSIRTYKKGMPIPFPWNKTIRLEAVKKESSSKHDEDEYEVYLNWYKSSRTEKKAIRFRLDFGKDKSNNQQIVKRCLNLDNTSSESYQLQTSSIQMKKGSEGAELYLLLVVNIPQDQHVLNKKIVVGVDLGINVPAYVATNCTEERKSIGDREHFLNARIAFHRRFHSFQKLKGTTGGRGRKKKLEPLERLREKERNWVHTQNHLISRDVINFALQTKAATIQMEDLSGYGKDEEGNVKPENKFLQSRWSYFELQSMIKYKAAKCGIKVNLINPSYTSQTCSWCGQMGVRESTSFVCQNPECKKYGKDIHADYNAARNIARSNKTVKNE

SEQ ID NO: 419 - protein, 478 aa, synthetic construct
  REGION 1..478: note = CasM. 287128 Effector Protein
MPTITRKIELRLCTEGLSDEERKAQWMLLYHINDNLYRSANNISSKLYLDEHVSSMVRLKHAEYQSTAAELLKAKKNNADEGTISTLEDKVETLKTEMSAQGIAICNYATEMATRTLAGKFASELELNIYGQILAEVKNVVHTNFTNDAKEVREGKRSIRTYKKGMPIPFPWNKSIKIEPVKASSQNEGQDDYEFYLKWYNGLKFILHFGKDRSNNRQILKRCFGLDNLCNERYQMRTSSIQMKKGSNGMELYLLLVLSIPKEQHSLNKKVVVGVDLGINVPAYVATNCTEERRAIGDREQFLNTRMAIIRRKHSFQRLKGTAGGRGRKKKLDPLERLRETERNWVHTQNHLYSRDIIKFALETKAATIQMEKLKGFGRDDNGNVIEEKKFLLGKWSYYELQNMIKYKAGKVGIKVNFIAPAYTSQTCSCCGVRDDRNRKSTSFICHNPDCQMYGKEIHADYNAARNIARSKNVIKDE

SEQ ID NO: 420 - protein, 468 aa, synthetic construct
  REGION 1..468: note = CasM. 287826 Effector Protein
MPAITRKIELTLCTEGLSEEQRKEQWGLLYHINDNLYKAANNISSKLYLDEHVSSMVRMKHADYLSLLKELARAEKQKTPDDELIAELREKLSLAEQEMTDQELAICNYATEMATSTLAYNFAKEIETEIFGQILACLENNAHAVFVDDSPTVRRGERAIRNYKKGMPIPFPWNKSIRIVEKDGEFYLRWYNGMRFLLTFGKDRSNNRIIMKRCLKMDQDFEGEYKLCNSSIQMVKREGKTKLFLLIVVNIPQEHVELNKNIVVGVDLGVNVPAYVATNITEERKAIGDREHFLNTRMQFQRRYKSLQRLKGTAGGKGRTKKLEPLERLRKAEHNWVHTQNHLFSREVVNFAVQTRAATIHMEDLSGFGKDNDGNADEQKEFVLRNWSFYELQNMIAYKAAKYGIKVEKVKPAYTSKTCSWCGQLGFRQGVTFICENPACKQCGEKVHADYNAARNIANSKDIIKKNE

SEQ ID NO: 421 - protein, 478 aa, synthetic construct
  REGION 1..478: note = CasM. 287896 Effector protein
MAGQRHTKVAKFQILKPAADMRWSELGRLLRDAQYRVYRLANLALSEKYLRFHLFRTGQTESLPECRIGRLNRQLRQMLKDEGGADDSVLDRFSRTGALPDTVVGALWQYRLHALTKGEKWNKVTRGETALPTFRRSMALPIRCDKRIHHRLERAALDSVELDLMICTRPYPRVILKTAKLDDGAAAILERLLDNEGQLLEGYRQRCFEVRYAEDEKAWWLHVTYDSPATPAPHLSKDIIVGVDLGFSCPMYVALSNGDARLGRRQFAALAARIRSLQTQVMARRRQMLSGGKASLSGDTARSGHGRKRKLLPIESLEGRINRAYTTLNHQLSISVVHFAVHHGAGVIQIENLEGLQNELTGTFLGQRWRYHQLQEFLNYKANEAGIEVRRVNPRYTSRRCSKCGYIHVDFNRAFRDAARQEGKVARFCCPKCEYEAHPDYNAARNLATVDIEGIIKVQCERQGIDRPSVENQDEVAK

SEQ ID NO: 422 - protein, 469 aa, synthetic construct
  REGION 1..469: note = CasM. 287936 Effector Protein
MPTITRKIELTLCTEGLSDQERKDQWNLLYHINDNLYRAANNISSKLYLDDHVGSMVRLKHAEYLSLLRALEKAKKQKAPDEEVIAELSQQVATAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNRLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKSTTGGRGRAKKLEPLERLRKAEQNWVHTQNHLFSREVIDFAVKARAATIHMEDLSGFGKDRDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 423 - protein, 469 aa, synthetic construct
  REGION 1..469: note = CasM. 288450 Effector Protein
MPTITRKIELSLCTDGLSDEQLKEQRQLLYHINDNLYRAANNVSSKLYLDEHVSSMVRLKHADYLSLLRDLARAEKQKSPDEALISELRSKLAAAQREMTEQELAICRYATEMSTQSLSYRFVTEMETHIFAKILDCLKQGVYATFNSDARDVKRGERSIRNYKKGMPIPFAWSDSVRIEQEADEFYLRWYNGIRFRLVFGKDRSNNRLIVKRCLKLDKDYEGDYKLCNSSVQMVKREGKPKTFLLLVVKIPQEQVELNKKIVLGVDLGINYPVYAATNCTEERIYFGEREHFLNTRMQFQRRYKSLQRLKGTTGGKGRKKKLEPLERLRKAERNWVHTQNHLFSQKTVDFALQTHAATIHLEDLSGFGRDSDGSAEEKKEFVLRNWSYYELQQMITYKAAKYGIKVEKIRPAYTSQTCSWCGQRGFRQGVTFICENPECKKCGEKEQADYNAARNIAKSKDVIKDDDE

SEQ ID NO: 424 - protein, 453 aa, synthetic construct
  REGION 1..453: note = CasM. 288712 Effector Protein
MSIVTRKIELIPDIENLTHEESNQRCYKLLYNIDKKLYKLANLLVCQLFGLDNLLSLMRLQNDEYVKFQSKLASKSISKETQKNIKEHMKEIDKELLARKAEIAPKSPLTFAYRAIKGSLYAKDLPSDIFNTLKQDVFKHFNETKKEQLRGERSLATYKRGIPIPFSLMKKNVIVSEGDNYYLTWFEETRFKLNFGKDRSNNRAIIDNCLKTNKYKLCTAAKIQLKNKKLFFLVTVDIPETKNTIIKGKVMGVDLGVVHPAYVAVNDGPERSLIGDGDAFQKQRDVFRRRFKELQRCQLTQGGHGRKHKTKATEILRGKERNWVQTENHRISRKIVNLAIRWKVESIQMENLKGFGKDSEGEVETKHKRLLGRWSYFELQKDIEYKAQKAGIKVVYINPAYTSQTCHVCGKKGDRTERDTFICLNTECSCYGKPQDADMNAAINIARSKNIVK

SEQ ID NO: 425 - protein, 468 aa, synthetic construct
  REGION 1..468: note = CasM. 289248 Effector Protein
MPTITRKIELMLCSEGLSDEQRKEQWGLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNKSLKIEAEGGDFYLRWYNGLRFLLTFGKDRSNNRMIVKRCMKMDEDFEGEYKLCNSSIQLAKRDGKPKLFLLLVVNIPQEHVELNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDMSGFGKDKDGNADEKKEFVLRNWSFYELQNMIAYKSAKYGIKVVKIRPAYTSKTCSWCGQQGERKSTTFICENPECKHYGESIHADYNAARNIANSNDIVKENE

SEQ ID NO: 426 - protein, 385 aa, synthetic construct
  REGION 1..385: note = CasM. 289726 Effector Protein
MVITRKIEVFVCESDNDLRRSYYEKLYDIRNIAQEAANRATSMLYAIDNLIPCLDEDSRKLIQYIGAKGTPASRQNAAYTIMSHLYKDRMPGIMDMLSNLAQYVTKNYSEDRKRGMYKNALRSYKCSLPVPYQKKSFKGLRFNWYEDSDGDAHEGCFFSLAGVPLQMRFGRDRSNNRLIVERVISGEYKMCTSSLKFDGKKLFLLLCVDIPKQEANVDPKKTLYAYLGVMNPIICTCDVRAKQEYDSGYKCFEIGTKEEFNYRRRQIQEAVRRCQINNRYSSGGKGRKKKCQAIERWHEKEKNYVDTKLHTYSRMLVDLAVAHKCGTIVLLNQKKREDKAKDDNQNGEPFVLRNWSYYNLKDKIGYKCKLAGIKLVQDKEETEEE

SEQ ID NO: 427 - protein, 372 aa, synthetic construct
  REGION 1..372: note = CasM. 289802 Effector Protein
MVITRKIEVFVCEDSKDLRKEYYDKIYKCRDIAVKTANLGVSHLFMLDNTTPYLSDDDREKLTFLGCSGKKATKQNAPYVAASEKFKGQADMSMLSSVLQNVGKMYQDDKKKGMWSKSLRSYKANMPIPFKASCYRNLRFADYNDKEDKPHNGCFFTLMGIPFQCKFGKDRSGNRIIMQAVVDGKYKMCTSSLQIDGKKIFLLLCVDIPKKVVKLDESKTLYAFLGVMNPIVCTTDIKQKGDIDTDWKLWEIGTEAEFNYRRRQIQEAVKRCQVNNRYSRGGHGRFAKTKAIERWRAVERNYVDTKLHTYSKMLIDLAVKHKCGKIVLMNQLHREDAAKDDKFVLRNWSYHSLRTKIDYKAKMYGIKVEVEK

SEQ ID NO: 428 - protein, 483 aa, synthetic construct
  REGION 1..483: note = CasM. 290380 Effector Protein
MPVITRKIKLNLCTEGLSEDERKAQWKMLYRINDNLYRAANNISSKLYLDEHVSSMVRLKNAEYTSLVSDLMKAKKAEDEAAITDLEAKIESLKSEMTAQEEAICCYATEMATRTLAGKFASELDLDIYGQILAEVKSVVFKNFNSDSKEVREGNRSIRTYKKGMPIPFPWNKTIRLEAVKKELSGKHDEDEYDFYLNWYKSSRTDKKAIRFRLYFGKDKSNNQQIVKRCLHLDSTSSENYQMQTSSIQMKKGPEGAELYLLLVVNIPQEQHALNKKIVVGVDLGINVPAYVATNCTEERKAIGDRDHFLNTRMAFSRRFHSFQRLKGTSGGKGRKKKLEPLERLREKERNWVHTQNHLISRDVINFALQVKAATIQMEDLSGYGKDEEGNVKPENKFLQSKWSYFELQSMIKYKAAKCGIKVNLIAPAYTSQTCSWCGQMGIRESTSFVCQNPECKQYGKDIHADYNAARNIARSNKIVKNE

SEQ ID NO: 429 - skipped

SEQ ID NO: 430 - protein, 478 aa, synthetic construct
  REGION 1..478: note = CasM. 293203 Effector Protein
MEEKTKRLQKVAKFQIVKPVNMTWVELGKMLRDVRYRLWRLANMAVCENYMRFYQWRIGKTDANENHKVKILNRRLREMIIEEKQADAKELMRYSRDGVVSGYICGAFEKIHLSAIKNKSKWREVIRGKSNLPLFKRDLPIPINCSDHKPSLIAKTESDEYEVDLRICQKPYPRVLLSTAKISGGERAILERLVSNKTNSLPGYRHRFFEIKEKPKGRWNLHVTYDFARSEATMLHSDIIVGVDLGWSVPLYAAVNKGHARIGWRKLEPLAKRIRHLQKQVKARRLSVQKGGQRDLAAPTARAGHGRKRILQPIEKLEGKIDDAYKTLNHQLSHCVIEFAKNNGAGVIQVENLEGLKDTLTGTFIGQNWRYNQLQNYIEYKAKEAGMELKKVNPCQTSQRCSNCGFIHRDFTFEYRQANKKNGKAAMFECPECSKKENYKPLNADYNAARNLATAGIEGKIRLQCEKQGIEYKGLPEE

SEQ ID NO: 431 - protein, 453 aa, synthetic construct
  REGION 1..453: note = CasM. 294190 Effector Protein
MSKITRKIEIIPDIEGLTHDESNKKCYGAFYTFDKNLYKVANLLVSQLYGLDNLLSLMRLQNDEYVKCQSKLSLKSTTDAEKENLKKRMKKIDAELVSIKNGMAPKHPQTFAYRAVTNCVYAKNIPSDILNTLKQDVYKHFNDTKKEQFLGERSLTTYKRGMPVPFSIEKKHAIVCDGDNYYLPWFEDTRFRLNFGRDKSNNRAIIDNCIKTKRYKLCAAAKIQLKDKKLFLLVTVDIPATETTSVKGKVMGVDLGVVNPAYVAVNDGPERSRIGNGEAFQKQRDVFRRRFRELQRSQLTQGGHGRKHKTKATETLRGKERNWVQTENHRISREIVNLASRWKVECIQMESLKGYGKNQEGEVEDNHKRLLGRWSYFELQKDIEYKAAMVGIQVKYINPAYTSQTCHVCGQRGNRIERDTFICTNPECTCYNQAQDADMNAAINIAKSKDVVK

SEQ ID NO: 432 - protein, 469 aa, synthetic construct
  REGION 1..469: note = CasM. 294406 Effector Protein
MPTITRKIEMKLCTEGLSDQERKDQWNLLYHINDNLYRAANNISSKLYLDDHVLSMVRLKHAEYLGLLRALEKAKKQKIPDEEVIAELSQKVAAAEQEMDDQAKAICQYATEMSTQSLSYRFATELETGIFTKILDCLKQGVFATFNSDTRDVKRGERSIRTYKKGMPIPFAWNDSLRIELEDGEFYLRWYNGLRFRFDFGKDRSNNRLIVRRCLNMDEDYEGDYKLCNSSIQMVKREGLAKFFLLMVVNIPQEQVELNKKIVVGVDLGINAPAYVATNITSERKQIGDREHFLNERMAFQRRFKSLQRLKGTTGGRGRAKKLEPLERLRKAEQNWVHTQNHLFSREVIDFAVKSRAATIHMEDLSGFGKDRDGNADDKKEFVLRNWSYYELQSMITYKAAKYGIKVEKIRPAYTSKTCSWCGHQGFREGITFICENPECKKYGEKEHADYNAARNIANSIEIVKNNEE

SEQ ID NO: 433 - skipped

SEQ ID NO: 434 - protein, 480 aa, synthetic construct
  REGION 1..480: note = CasM. 294655 Effector Protein
MPFKVLKLKIIKPVNMDWNELGQSIRDTRYRVYRLANLAVSEAYLAFHLWRAGKTDAIPKATAGQLNRRLRDMLLEEARTKAVKDRKNTGEKGTEDDAKKAQKEMNKFSKTGALPDTVAGALFMYKVKGLISKGKWTQVIRGKSALPTFRNNMAIPIRCDKKTQRRLERTENGVELELMIRNKPYPRVLLGTQGIGEGAEAIIERLLSNESQAEQGYKQRYFEVREDVNRTWWLYVCYALPASTPPRLDPSKIVGVDLGFTCPMYAAISNGHARLGYRAFSSLAARVKALKLRTMRRRREIQRGGRTIVSGEAARSGHGRKRKLLGIEKLQGRVNQAYTTLNHQMSAAVVKFAIENGAGTIQVENLEGLREELSGTFLGQMWRYFQLQEFLQYKAEENGIVIRKVNPRYTSRRCSQCGHINKEFTRKARDRNAEGGYSAKFKCPDCEYEADADYNAAKNLAVDGIEGIIEKQCGSQGIVL

SEQ ID NO: 435 - protein, 494 aa, synthetic construct
  REGION 1..494: note = CasM. 295201 Effector Protein
MFLYKELKTMAKTNAEEGKIENKEKRLTKVAKFQIVKPVNMTWPEFGKMLGDVRYRLSRVANMAVTEKYLESQQKRTGQKIQRENTLVTIANRKLREMLKKEKVKEEELDRYSRDGAVSGYVTGPFEHNKLSAISKRFKEVLKGNMSLPNFKREMAIPINCSNAKLSTIEKTETGEYVVDLRISQKPWPRVLLSTNRISNGQREILERLAANKTFSDDGYKHLFFEVKQQGKDWFLSVTYSFPKSEAPKLHKDIIVGVDLGWSVPLYAAVNKGYARIGWQKFRPLAERIKHLQKQVKARRITIQKGGQQDLATPTARTGHGRKRILRPIEKLERKIENAYTTLNHQLSHCVIEFAKNNGAGVIQIENLSGLANELSGTYIGQNWRYEQLQEYIRYKAEEAGIEVKHVNPCRTSQRCSECGFINDKFNFEYRQANRNNGMSAMFECPECKKNKKDYKPINADYNAAKNLTTANIDEIIRLQCKKQGIEYKELPKD

SEQ ID NO: 436 - protein, 453 aa, synthetic construct
  REGION 1..453: note = CasM. 296640 Effector Protein
MSKITRKIELIPDIENLTHEESNQRCYKVFYNIDNKLYKVANLLVCQLFGLDNLLSLMRLQNDEYVKCQSKLASKSISEETKRDIKKRMEAIDKELLARKDEIAPKHPQTFAYRAIKDSDYAKDLPSDIFNTLKQDVFKHFNETKKEQLRGERSLTTYKRGIPVPFNLMKKNVIVSDGDNYYLTWFEETRFKLNFGKDRSNNRAIIDNCLKTNKYKLCTAAKIQLKNKKLFLLVTVDIPETKNKIIKGKVMGVDLGVVHPAYVAVNDGPERSLIGDGDAFQKQRDVFRRRFRELQRCQLTQGGHGRKHKTKATENLRGKERNWVQTENHRISREIVNLAIRWRVETIQMENLKGFGKDSDGDVETKHQRLLGRWSYFELQKDIEYKAAMAGIKVVYVNPAYTSQTCHVCGERGDRTERDTFICTNTECDCYGKPQDADMNAAINIARSKNIVK

SEQ ID NO: 437 - protein, 386 aa, synthetic construct
  REGION 1..386: note = CasM. 296642 Effector Protein
MTKVVKLPLICEQSDKDGNPIDYKKIYEILFELQRQTREIKNKSIQYCWEFSNFSSDYYKQNHEYPKEKDILSYTLVGFVNDKFKTGNDLYSGNCSTTVRGACGEFKNSKTDFLKGTKSIINYKGNQPLDLHNKNSSKTIVERCIDNIYKISASKLIYNEKKKCWVLNLSYSFTNNNVCELDENKILGVDLGIHYPICASVNGERKFFKIDGGEIDHTRRKIEVRKKSLLKQGSSCGEGRIGHGIKTRNKPVYNIEDKIACFRDTANHKYSRALINYAVNNNCGIIQMEKLTGITADSDRFLKNWSYFDLQTKIEYKAKEAGITVVYIDPQYTSQRCSKCGYISKENRKVQAKFCCQKCGYEANADYNASQNIGIKDIDKIIKNTK

SEQ ID NO: 438 - skipped

SEQ ID NO: 439 - protein, 1331 aa, synthetic construct
  REGION 1..1331: note = CasM. 298248 Effector Protein
MNRIYQGRVSKVEIPDGKDEWKKLDDGESALWQHHQLFQDDVNYLLAAFAALVPTSCEDDIWKDYQAAIERSWESYTGRQGIWDRPFENACVIVGCKKDASFKEFRRKLNSLTGSKASEKQKFEALKQLFEPATEAAKKLKKHDEPVEESLKGKAKDLFGSTLVNLCAQKTKVTPRDVIAKQRNRASECTKKVNEGERLKWADVFYFKTDTSAAKWSREDAAKNIIQFLDKLLGEVEEKEKDAKTSDQKKKMADLAERLEKQKKPLAAWCNNSKTDLPTTEPTRKGSGGYDLKAAVLFSLQPDLDGFRDAFLLFNQARLKEEFATTEKGDAAYIARMAGGVARPVFPFFCDVWAGKVNDEKIGQGIWPDFEKQAFSEVFTKIGQFIVRGRKFELRLAIADQIIAKIETQKKSDARLQAVERIAEDLADELPDTAVDENGQKRPYGIRERTLKGWRKVRPAWREALKKTPNLTAEDLIKQKNRMQERQREKYGSASLFDRLAKEPEIWNHDDKEDAVETWADYVENLEEKAHLETERLFAPAHATLSPRFFRWSETNNKEHLEASSPDVPFELKADALDLSKKEKSQIKIHFWSPRLWRDGLRGKKENLDKDEPDQNWMPPVLRAFVKARKWPCDKQSFAGASVRLAPRCKENIQLVFEPELHTEILSAKWKENFPFSPAKNKESESVGLFWPRTKEDKVLWFDKGETRCLGVDLGLTNSAAWQILQATNKDATAKAPRLRHRLNPDSEKAAWFAHSITNGIVRVAGEDCWGWRKFAPDEKAKLRAELKKPAGKRNALCRKFLSLNREIEFETATHSFLPELSGSGGRNPTDDETKEAAEFFSTLKTKGFDITDRQPSWGKNLSFPKQNDELLWGLKRVRAQLFRLNRWSEQLGKERDSKPYQSAIEIIGNLRSDDPLIELATLKSEPKRLKSRIAELAGEYLDCFKTLLPRIADRILPWRRGHWSWKPCDNDWHRMELDASKPRPEALLAGQRGISLPRLNQLKDLRQLAQSLNHLCRRKQIKRNETVPEPFEDCRQAMEDAREDRAKKIAHEVFAIALGVELAPPPPDKQERKQTESLHGVYRCLERGPVNFIALENLGGYNPSAKQGRRENRQLSSWLKGRIHKILGELCEMVGMPIVLVNAEYTSRFSAKDHSPGFRAEEVQTDDSRRSFWQRKAKEEPSGWQNEFLCWLNKVPDGKSLLLPKKGGEFFVPLGEGTSLYHADLNAAYRIALRALAHRDRAELLGQTWIEKKPYLVDVAGVFPDSILRNGCAFKTISSSERLWEKVNGDLAMQRCREINLARFASWKIALPQQIISEALPPDEEDDIPM

SEQ ID NO: 440 - protein, 517 aa, synthetic construct
  REGION 1..517: note = CasM. 298264 Effector Protein
MSEATKTLAYRYRLRLTPAQEDILDRSQEQLRLVWNHLVRSQHKVEHEWRHGRAASIKNELLELSLAKNATGQAIPSARKITEERGVSMEEALRLMRQKFVEKVSAIPLRKKDGSRCLRIARRKMATEYAVTVVNAKFKHYYGLGARMCKVLRDKFQKCSDMWIKGKFRRPRFKRKGESVALQRQVQSNSPFKLKRFSDLSALGGQALKKCEVIIHRPLPDSAEIKQIAVSGRRGQRHLIVMFKAASSDVAKNFPATNRTAGVDPGIKVALTITPLDSPDFGTSDKIEKQPDLARDACFLKRLRRLQRKHDRQRRQNNPECFDEKGRWIKGKRLHNESKNMQRTQSRITAMNTHLAESRRDFYHNAACEILRSFDNVAVGKWRPAQTRQRKPTTPSPKGLGAARRATNRISYDHAISLFISYLKDKAERSVTTKHVQEVSEFGSTRSCPKCGKLTGPVGTEGLAVRDWTCVNCNTTFQRDAASAWQIAKRFKAEVASTSQPAESQDSANSASVLTQV

SEQ ID NO: 441 - protein, 470 aa, synthetic construct
  REGION 1..470: note = CasM. 298446 Effector Protein
MPTLTRKVELYVVGDKEEVSRVYDYIRLAMNATYKCFNECMTALYIAQVKEDTKEDRKELNHLYSRQTYTKKETAFTNDIVFPEGLALAAYVNRMAQQKFVTSLKNGLMYGCVSLPTFKKDCAVPLHVKFVSLAGEKGTNTGFYHEYADVNDLVNALEYDNSPKVFLRFPNNITFGVVFGNPYRGREQRSVFSKIFLGEYKIQGSSIQINSRGKIILNLSMEVPKKKMEHIEGRVVGVDVGLAIPAMCAINDDDYTRSAIGNIDDFLKVRTQIQSQRRRLQKSLKNTSSGHGRTKKLKPLERIAEKERNFANTYNHMVSKRVVDFAVKNGASQINIEDLSGFAKDKNGKSVEDDNMKRVLSNWSYFELQQQIRYKAEQYDIKVRTVNPAYTSQTCSYCGQIGKRETQSKFVCTNPDCKCHKMYKKDWFNADFNAARNIALSTDYTDDEDGKKTKKKKSAKKKPEKKTEEA

SEQ ID NO: 442 - protein, 500 aa, synthetic construct
  REGION 1..500: note = CasM. 298612 Effector Protein
MSGASGQITRDNKAQRSGPNKGEMSEDHSSTKRPKRVVKVAKYRIIKPVGEMTWPELGEILRTVRYRVFRLGNLAVSEAYLNFHAFRTGKAEEFKSETIGKLSRRLRDMLISEGVKKEDIDRYSATGAVPDTVAGALGQYKVRGITSPAKWRQVIRGTVSLPTFRNDMAIPVRCDKPAQRRLEKAKSEEVEVDLMICRKPYPRVLIGTADLGGGQQAILERLLDNKDNSSDGYRQRLFEIKQDTQSKKWFLFVTYDFPSSGALPLDPNVAVGVDLGVSVPLYAAINNGHARLGRRQFQALGSRIRSLQTQVDARRRAIQRGGRSDVSQSTARSGHGVRRKLQPTEKLRKRIDRSYSTLNHQLSAAVVEFAKNQGAGTVQMEDLGGLREELTGTFIGARWRYHQLQQFLEYKCDEAGITLNKVNPMYTSRRCSECGFIDKDFDRAFRDRSRSDGRVARFICPECSYEADPDYNAARNIATLDIDKLIRVQCQKQGLKYDAL

SEQ ID NO: 443 - protein, 459 aa, synthetic construct
  REGION 1..459: note = CasM. 299584 Effector Protein
MIITRKIELWLSEDDNELRKAKWSYLKELNDEVYRAANFIVNNQYFNEILENRVIMQDTRLIDIDSEIRKLYKSREKNKEKIDELKKIKKIRYQEAKNFYQTSKQNVTYQLTSREFPNIPANIVTSLNASIIKTLKTEWNEIKSGKRAVRNYRKGMPIPFNFSSSQKWFENKGEDIFLNWLGGLKFKLFFGRDKSNNRAIVERAINKEYKYADSSIQLKDKKIFLLFVVDIPYEKANLNKNIAAGVDLGIAFPAFCALSEGYSRLSIGNKEDLLKVRLQMQSRRKRLQKALKITSGGKGRTKKLKALESLTNKEKNYVTTYNHKVSYQVIKFAKDNKAGIIKLEFLEGFGEDEKNKFILRNWSFYQLQKMIEYKAKREGIEVLYIDPYHTSQTCAICGNYEEGQREKQEDFICKNPECKNFEKIVNADYNAALNIAKSNKIVSSSEQCEYNKKHENNVL

SEQ ID NO: 444 - protein, 469 aa, synthetic construct
  REGION 1..469: note = CasM. 286251 (D267A) Effector Protein
MPTITRKIELTLCTEGLSDQERKDQWNLLYHINDNLYRAANNISSKLYLDDHVGSMVRLKHAEYLSLLRALEKAKKQKAPDEEVIAELSQQVATAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNCLIVQRCMKMDKDYEGDYKLCNSSIQMVKREGKPKFFLLLVVNIPQERVELNKNIVVGVALGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDNDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 445 - protein, 468 aa, synthetic construct
  REGION 1..468: note = CasM. 19952 (D267A) Effector Protein
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVALGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 446 - protein, 468 aa, synthetic construct
  REGION 1..468: note = CasM. 19952 (D267N) Effector Protein
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVNLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 447 - protein, 468 aa, synthetic construct
  REGION 1..468: note = CasM. 19952 (E363Q) Effector Protein
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMQDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 448 - protein, 50 aa, synthetic construct
  REGION 1..50: note = Prosite motif
  SITE 1: note = MISC_FEATURE - Xaa = K or G
  SITE 2: note = MISC_FEATURE - Xaa = E or T
  SITE 4: note = MISC_FEATURE - Xaa = V or F or L
  SITE 5: note = MISC_FEATURE - Xaa = L or G
  SITE 6: note = MISC_FEATURE - Xaa = R or K
  SITE 9: note = MISC_FEATURE - Xaa = S or T or R
  SITE 11: note = MISC_FEATURE - Xaa = Y or F
  SITE 12: note = MISC_FEATURE - Xaa = E or D or Q
  SITE 15: note = MISC_FEATURE - Xaa = N or T
  SITE 16: note = MISC_FEATURE - Xaa = M or K
  SITE 18: note = MISC_FEATURE - Xaa = E or K
  SITE 22: note = MISC_FEATURE - Xaa =K or A
  SITE 24: note = MISC_FEATURE - Xaa =Y or A
  SITE 29: note = MISC_FEATURE - Xaa =V or E
  SITE 30: note = MISC_FEATURE - Xaa =K or Y
  SITE 31: note = MISC_FEATURE - Xaa =I or V
  SITE 32: note = MISC_FEATURE - Xaa =N or R
  SITE 34: note = MISC_FEATURE - Xaa =A or K
  SITE 38: note = MISC_FEATURE - Xaa =Q or R or K
  SITE 39: note = MISC_FEATURE - Xaa = R or T
  SITE 42: note = MISC_FEATURE - Xaa = W or K
  SITE 45: note = MISC_FEATURE - Xaa = Y or Q or H
  SITE 47: note = MISC_FEATURE - Xaa = G or D
  SITE 48: note = MISC_FEATURE - Xaa = K or F
  SITE 49: note = MISC_FEATURE - Xaa = R or D
  SITE 50: note = MISC_FEATURE - Xaa = N or F
XXFXXXNWXYXXLQXXIXYKAXEXGIKVXXXXPXYTSXXCSXCGXIXXXX

SEQ ID NO: 449 - protein, 29 aa, synthetic construct
  REGION 1..29: note = Prosite motif
  SITE 2: note = MISC_FEATURE - Xaa = Q or L
  SITE 5: note = MISC_FEATURE - Xaa = L or R or Q
  SITE 6: note = MISC_FEATURE - Xaa = Y or F
  SITE 9: note = MISC_FEATURE - Xaa = E or A
  SITE 10: note = MISC_FEATURE - Xaa = V or L
  SITE 11: note = MISC_FEATURE - Xaa = I or V
  SITE 12: note = MISC_FEATURE - Xaa = D or E or N
  SITE 13: note = MISC_FEATURE - Xaa = F or Y
  SITE 17: note = MISC_FEATURE - Xaa = N or H
  SITE 17: note = MISC_FEATURE - Xaa = N or H
  SITE 20: note = MISC_FEATURE - Xaa = G or A
  SITE 23: note = MISC_FEATURE - Xaa = Q or H
  SITE 26: note = MISC_FEATURE - Xaa = D or N
TXNHXXSRXXXXXAVKXGAXTIXMEXLSG

SEQ ID NO: 450 - protein, 21 aa, synthetic construct
  REGION 1..21: note = Prosite motif
  SITE 2: note = MISC_FEATURE - Xaa = N or D
  SITE 3: note = MISC_FEATURE - Xaa = K or P
  SITE 4: note = MISC_FEATURE - Xaa = N or K or E
  SITE 5: note = MISC_FEATURE - Xaa = I or K
  SITE 6: note = MISC_FEATURE - Xaa = V or I
  SITE 7: note = MISC_FEATURE - Xaa = V or L
  SITE 13: note = MISC_FEATURE - Xaa = I or V
  SITE 14: note = MISC_FEATURE - Xaa = N or S
  SITE 15: note = MISC_FEATURE - Xaa = V or Y
  SITE 17: note = MISC_FEATURE - Xaa = L or A
  SITE 19: note = MISC_FEATURE - Xaa = A or V
  SITE 20: note = MISC_FEATURE - Xaa = A or S
  SITE 21: note = MISC_FEATURE - Xaa = T or V
LXXXXXXGVDLGXXXPXYXXX

SEQ ID NO: 451 - protein, 41 aa, synthetic construct
  REGION 1..41: note = Prosite motif
  SITE 3: note = MISC_FEATURE - Xaa = G or N
  SITE 14: note = MISC_FEATURE - Xaa =K or R
  SITE 27: note = MISC_FEATURE - Xaa = D or E
  SITE 35: note = MISC_FEATURE - Xaa = L or M
  SITE 38: note = MISC_FEATURE - Xaa = A or D
QWXLLYHINDNLYXAANNISSKLYLDXHVSSMVRXKHXEYL

SEQ ID NO: 452 - protein, 21 aa, synthetic construct
  REGION 1..21: note = Prosite motif
  SITE 2: note = MISC_FEATURE - Xaa = L or K
  SITE 5: note = MISC_FEATURE - Xaa = E or K
  SITE 7: note = MISC_FEATURE - Xaa = S or A
  SITE 8: note = MISC_FEATURE - Xaa = I or L
  SITE 9: note = MISC_FEATURE - Xaa = P or R
  SITE 10: note = MISC_FEATURE - Xaa = N or T or S
  SITE 11: note = MISC_FEATURE - Xaa = Y or F
  SITE 12: note = MISC_FEATURE - Xaa = K or R
  SITE 13: note = MISC_FEATURE - Xaa = K or S
  SITE 14: note = MISC_FEATURE - Xaa = G or D or N
  SITE 15: note = MISC_FEATURE - Xaa = M or Q
  SITE 17: note = MISC_FEATURE - Xaa = I or L
  SITE 19: note = MISC_FEATURE - Xaa = F or I
  SITE 20: note = MISC_FEATURE - Xaa = H or P
  SITE 21: note = MISC_FEATURE - Xaa = W or C
VXRGXRXXXXXXXXXPXPXXX

SEQ ID NO: 453 - protein, 15 aa, synthetic construct
  REGION 1..15: note = Prosite motif
  SITE 1: note = MISC_FEATURE - Xaa = N or H
  SITE 7: note = MISC_FEATURE - Xaa = A or S
  SITE 8: note = MISC_FEATURE - Xaa = R or Q
  SITE 10: note = MISC_FEATURE - Xaa = I or L
  SITE 11: note = MISC_FEATURE - Xaa = A or S
  SITE 12: note = MISC_FEATURE - Xaa = I or N
  SITE 13: note = MISC_FEATURE - Xaa = S or K
  SITE 14: note = MISC_FEATURE - Xaa = K or D
  SITE 15: note = MISC_FEATURE - Xaa = I or D
XADYNAXXNXXXXXX

SEQ ID NO: 454 - protein, 23 aa, synthetic construct
  REGION 1..23: note = Prosite motif
  SITE 1: note = MISC_FEATURE - Xaa = R or Y
  SITE 2: note = MISC_FEATURE - Xaa = L or C
  SITE 3: note = MISC_FEATURE - Xaa = G or K
  SITE 4: note = MISC_FEATURE - Xaa = G or T
  SITE 5: note = MISC_FEATURE - Xaa = T or G
  SITE 7: note = MISC_FEATURE - Xaa = G or I
  SITE 9: note = MISC_FEATURE - Xaa = H or K
  SITE 13: note = MISC_FEATURE - Xaa = K or R
  SITE 14: note = MISC_FEATURE - Xaa = K or R
  SITE 18: note = MISC_FEATURE - Xaa = L or I
  SITE 19: note = MISC_FEATURE - Xaa = E or Y
  SITE 20: note = MISC_FEATURE - Xaa = R or K
  SITE 22: note = MISC_FEATURE - Xaa = R or E
  SITE 23: note = MISC_FEATURE - Xaa = D or G
XXXXXRXGXGRKXXLEPXXXLXX

SEQ ID NO: 455 - protein, 20 aa, synthetic construct
  REGION 1..20: note = Endosomal Escape Protein
  SITE 4: note = MISC_FEATURE - X is independently selected from lysine, histidine, and arginine
  SITE 8: note = MISC_FEATURE - X is independently selected from lysine, histidine, and arginine
  SITE 11: note = MISC_FEATURE - X is independently selected from lysine, histidine, and arginine
  SITE 15: note = MISC_FEATURE - X is independently selected from lysine, histidine, and arginine
  SITE 19: note = MISC_FEATURE - X is independently selected from lysine, histidine, and arginine
GLFXALLXLLXSLWXLLLXA

SEQ ID NO: 456 - protein, 20 aa, synthetic construct
  REGION 1..20: note = Endosomal Escape Protein
GLFHALLHLLHSLWHLLLHA

SEQ ID NO: 457-462 - skipped (6 entries)

SEQ ID NO: 463 - DNA, 182 bp, synthetic construct
  misc_feature 1..182: note = Amplicon Sequence
  source 1..182: mol_type = other DNA
acacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgt

SEQ ID NO: 464 - DNA, 231 bp, synthetic construct
  misc_feature 1..231: note = Amplicon Sequence
  source 1..231: mol_type = other DNA
tctagggatggtggcttctggaaggctgaccatgcacaggcctccaatccctccccctggcctctgtttccgacagcttgtacaataactgcatctgcgacgtgggagccgagagcttggctcgtgtgcttccggacatggtgtccctccgggtgatggagtgagtgtgggagtctgggcggtgggtggctcagcccggggtgggagacactgaagtctctccctggtgtc

SEQ ID NO: 465 - DNA, 260 bp, synthetic construct
  misc_feature 1..260: note = Amplicon Sequence
  source 1..260: mol_type = other DNA
ggatgggaagggtcagatggccccaggacgctagctgatggcccccatctgattccacctgcagcctggatgcgctgagtgagaacaagatcggggacgagggtgtctcgcagctctcagccaccttcccccagctgaagtccttggaaaccctcaagtgagtgagctgggcctgcccttcctgctgaatcgggcccccaaagtccggctgactttttcaaaattaatttaaatttgtttttttagacaagggctcgctg

SEQ ID NO: 466 - DNA, 255 bp, synthetic construct
  misc_feature 1..255: note = Amplicon Sequence
  source 1..255: mol_type = other DNA
gcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggtt

SEQ ID NO: 467 - DNA, 251 bp, synthetic construct
  misc_feature 1..251: note = Amplicon Sequence
  source 1..251: mol_type = other DNA
aggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgc

SEQ ID NO: 468 - DNA, 251 bp, synthetic construct; identical to SEQ ID NO: 467
  misc_feature 1..251: note = Amplicon Sequence
  source 1..251: mol_type = other DNA

SEQ ID NO: 469 - DNA, 225 bp, synthetic construct
  misc_feature 1..225: note = Amplicon Sequence
  source 1..225: mol_type = other DNA
aatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctcc

SEQ ID NO: 470 - DNA, 266 bp, synthetic construct
  misc_feature 1..266: note = Amplicon Sequence
  source 1..266: mol_type = other DNA
cccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactg

SEQ ID NO: 471 - DNA, 236 bp, synthetic construct
  misc_feature 1..236: note = Amplicon Seqeunce
  source 1..236: mol_type = other DNA
agcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacat

SEQ ID NO: 472 - DNA, 233 bp, synthetic construct
  misc_feature 1..233: note = Amplicon Sequence
  source 1..233: mol_type = other DNA
cctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctc

SEQ ID NO: 473-480 - skipped (8 entries)

SEQ ID NO: 481 - RNA, 33 nt, synthetic construct
  misc_feature 1..33: note = Repeat Sequence
  source 1..33: mol_type = other RNA
cgttgcagctcgcacgttggcactggttgaagg

SEQ ID NO: 482 - RNA, 34 nt, synthetic construct
  misc_feature 1..34: note = Repeat Sequence
  source 1..34: mol_type = other RNA
cgttgcagctcgcacgttggcactgggttgaagg

SEQ ID NO: 483 - RNA, 17 nt, synthetic construct
  misc_feature 1..17: note = Repeat Sequence
  source 1..17: mol_type = other RNA
ttggcactggttgaagg

SEQ ID NO: 484 - RNA, 13 nt, synthetic construct
  misc_feature 1..13: note = Repeat Sequence
  source 1..13: mol_type = other RNA
cactggttgaagg

SEQ ID NO: 485 - RNA, 32 nt, synthetic construct
  misc_feature 1..32: note = Repeat Sequence
  source 1..32: mol_type = other RNA
gttgcaactcacgcgcgtatgtggcttgaagg

SEQ ID NO: 486 - RNA, 32 nt, synthetic construct
  misc_feature 1..32: note = Repeat Sequence
  source 1..32: mol_type = other RNA
gttgcaattcatatctccgggtggattgaagg

SEQ ID NO: 487 - RNA, 32 nt, synthetic construct
  misc_feature 1..32: note = Repeat Sequence
  source 1..32: mol_type = other RNA
gttgcagcgtgcgcgagcgtgtggcttgaagg

SEQ ID NO: 488 - RNA, 17 nt, synthetic construct
  misc_feature 1..17: note = Repeat Sequence
  source 1..17: mol_type = other RNA
agcgtgtggcttgaagg

SEQ ID NO: 489 - RNA, 13 nt, synthetic construct
  misc_feature 1..13: note = Repeat Sequence
  source 1..13: mol_type = other RNA
tgtggcttgaagg

SEQ ID NO: 490 - RNA, 32 nt, synthetic construct
  misc_feature 1..32: note = Repeat Sequence
  source 1..32: mol_type = other RNA
gttgcaactcgcacgtgaatgcgacttgaagg

SEQ ID NO: 491 - RNA, 17 nt, synthetic construct
  misc_feature 1..17: note = Repeat Sequence
  source 1..17: mol_type = other RNA
tgaatgcgacttgaagg

SEQ ID NO: 492 - RNA, 32 nt, synthetic construct
  misc_feature 1..32: note = Repeat Sequence
  source 1..32: mol_type = other RNA
gatgcaactcgtgtgtatgtgcgagttgaagg

SEQ ID NO: 493 - RNA, 32 nt, synthetic construct
  misc_feature 1..32: note = Repeat Sequence
  source 1..32: mol_type = other RNA
gacgcaactcgcgcgcgggcatgtattgaggg

SEQ ID NO: 494 - RNA, 32 nt, synthetic construct
  misc_feature 1..32: note = Repeat Sequence
  source 1..32: mol_type = other RNA
gatgcatctgacacagctgggtgagttgaagg

SEQ ID NO: 495 - RNA, 17 nt, synthetic construct
  misc_feature 1..17: note = Repeat Sequence
  source 1..17: mol_type = other RNA
gctgggtgagttgaagg

SEQ ID NO: 496 - RNA, 32 nt, synthetic construct
  misc_feature 1..32: note = Repeat Sequence
  source 1..32: mol_type = other RNA
gttgcaacacatgtatgtgggtgagttgaagg

SEQ ID NO: 497 - RNA, 32 nt, synthetic construct
  misc_feature 1..32: note = Repeat Sequence
  source 1..32: mol_type = other RNA
gttgcaatttgtatacgagtgtgacttgaagg

SEQ ID NO: 498 - RNA, 32 nt, synthetic construct
  misc_feature 1..32: note = Repeat Sequence
  source 1..32: mol_type = other RNA
gctgcaacacgcgcgggtacgcgggttgaagg

SEQ ID NO: 499 - RNA, 32 nt, synthetic construct
  misc_feature 1..32: note = Repeat Sequence
  source 1..32: mol_type = other RNA
gttgcaactcgcacgttggcactgattgaagg

SEQ ID NO: 500 - RNA, 36 nt, synthetic construct
  misc_feature 1..36: note = Repeat Sequence
  source 1..36: mol_type = other RNA
gctgtagccctgctcaaattgtagggcgcatgcagg

SEQ ID NO: 501 - RNA, 36 nt, synthetic construct
  misc_feature 1..36: note = Repeat Sequence
  source 1..36: mol_type = other RNA
gttgtagtcgacctgaatctgtggggtgcttacagg

SEQ ID NO: 502 - RNA, 17 nt, synthetic construct
  misc_feature 1..17: note = Repeat Sequence
  source 1..17: mol_type = other RNA
tgtggggtgcttacagg

SEQ ID NO: 503 - RNA, 36 nt, synthetic construct
  misc_feature 1..36: note = Repeat Sequence
  source 1..36: mol_type = other RNA
ggtgtatgtaaccgcaatttgaagggtgcatacagg

SEQ ID NO: 504 - RNA, 36 nt, synthetic construct
  misc_feature 1..36: note = Repeat Sequence
  source 1..36: mol_type = other RNA
gttggaatcgaccttaatttgaggtgtgcttacagg

SEQ ID NO: 505 - RNA, 36 nt, synthetic construct
  misc_feature 1..36: note = Repeat Sequence
  source 1..36: mol_type = other RNA
gctgaaagagcagagaatttgttgtgtgcatacagg

SEQ ID NO: 506 - RNA, 36 nt, synthetic construct
  misc_feature 1..36: note = Repeat Sequence
  source 1..36: mol_type = other RNA
gttggagtcggcttgaatctgcggggtgcttacagg

SEQ ID NO: 507 - RNA, 24 nt, synthetic construct
  misc_feature 1..24: note = Repeat Sequence
  source 1..24: mol_type = other RNA
cctgattgctcgctgcggcgagac

SEQ ID NO: 508 - RNA, 36 nt, synthetic construct
  misc_feature 1..36: note = Repeat Sequence
  source 1..36: mol_type = other RNA
actgtcagacaatgcaaaatgtgtggtacatccaac

SEQ ID NO: 509 - RNA, 10 nt, synthetic construct
  misc_feature 1..10: note = Repeat Sequence
  source 1..10: mol_type = other RNA
tggtacatcc

SEQ ID NO: 510 - RNA, 65 nt, synthetic construct
  misc_feature 1..65: note = Repeat Sequence
  source 1..65: mol_type = other RNA
tggggcagttggttgcccttagcctgaggcatttattgcactcgggaagtaccattacatccaac

SEQ ID NO: 511 - RNA, 23 nt, synthetic construct
  misc_feature 1..23: note = Repeat Sequence
  source 1..23: mol_type = other RNA
tggtacatccaactctaggcgcc

SEQ ID NO: 512 - RNA, 15 nt, synthetic construct
  misc_feature 1..15: note = Repeat Sequence
  source 1..15: mol_type = other RNA
aatggtacatccaac

SEQ ID NO: 513 - RNA, 19 nt, synthetic construct
  misc_feature 1..19: note = Repeat Sequence
  source 1..19: mol_type = other RNA
tggtacatccaactctagg

SEQ ID NO: 514 - RNA, 22 nt, synthetic construct
  misc_feature 1..22: note = Repeat Sequence
  source 1..22: mol_type = other RNA
tggtacatccaactctaggcgc

SEQ ID NO: 515 - RNA, 21 nt, synthetic construct
  misc_feature 1..21: note = Repeat Sequence
  source 1..21: mol_type = other RNA
tggtacatccaactctaggcg

SEQ ID NO: 516 - RNA, 19 nt, synthetic construct
  misc_feature 1..19: note = Repeat Sequence
  source 1..19: mol_type = other RNA
tggtacatccaactctagg

SEQ ID NO: 517 - RNA, 16 nt, synthetic construct
  misc_feature 1..16: note = Repeat Sequence
  source 1..16: mol_type = other RNA
aaatggtacatccaac

SEQ ID NO: 518 - RNA, 16 nt, synthetic construct
  misc_feature 1..16: note = Repeat Sequence
  source 1..16: mol_type = other RNA
tggtacatccaactct

SEQ ID NO: 519 - RNA, 15 nt, synthetic construct
  misc_feature 1..15: note = Repeat Sequence
  source 1..15: mol_type = other RNA
tggtacatccaactc

SEQ ID NO: 520 - RNA, 14 nt, synthetic construct
  misc_feature 1..14: note = Repeat Sequence
  source 1..14: mol_type = other RNA
tggtacatccaact

SEQ ID NO: 521 - RNA, 18 nt, synthetic construct
  misc_feature 1..18: note = Repeat Sequence
  source 1..18: mol_type = other RNA
tggtacatccaactctag

SEQ ID NO: 522 - RNA, 13 nt, synthetic construct
  misc_feature 1..13: note = Repeat Sequence
  source 1..13: mol_type = other RNA
tggtatatccaac

SEQ ID NO: 523 - RNA, 17 nt, synthetic construct
  misc_feature 1..17: note = Repeat Sequence
  source 1..17: mol_type = other RNA
tggtacatccaactcta

SEQ ID NO: 524 - RNA, 14 nt, synthetic construct
  misc_feature 1..14: note = Repeat Sequence
  source 1..14: mol_type = other RNA
atggtacatccaac

SEQ ID NO: 525 - RNA, 12 nt, synthetic construct
  misc_feature 1..12: note = Repeat Sequence
  source 1..12: mol_type = other RNA
tggtacatccaa

SEQ ID NO: 526 - RNA, 11 nt, synthetic construct
  misc_feature 1..11: note = Repeat Sequence
  source 1..11: mol_type = other RNA
tggtacatcca

SEQ ID NO: 527 - RNA, 36 nt, synthetic construct
  misc_feature 1..36: note = Repeat Sequence
  source 1..36: mol_type = other RNA
gctgtcagtagtagtaaaaatgggggtacatccaac

SEQ ID NO: 528 - RNA, 36 nt, synthetic construct
  misc_feature 1..36: note = Repeat Sequence
  source 1..36: mol_type = other RNA
actgtcagtacatgcaaaaatgagggtacatccaac

SEQ ID NO: 529 - RNA, 36 nt, synthetic construct
  misc_feature 1..36: note = Repeat Sequence
  source 1..36: mol_type = other RNA
actgtcagacaatgcaaaatgagtggtacatccaac

SEQ ID NO: 530 - RNA, 36 nt, synthetic construct
  misc_feature 1..36: note = Repeat Sequence
  source 1..36: mol_type = other RNA
gctgttagaacatacaaaatgaaaggtacatccaac

SEQ ID NO: 531 - RNA, 36 nt, synthetic construct
  misc_feature 1..36: note = Repeat Sequence
  source 1..36: mol_type = other RNA
gctgcatgtcatggcaaaaggaaaggtacatccaac

SEQ ID NO: 532 - RNA, 36 nt, synthetic construct
  misc_feature 1..36: note = Repeat Sequence
  source 1..36: mol_type = other RNA
gctgtcagacacctaaaaaatgagggtacatccaac

SEQ ID NO: 533 - RNA, 36 nt, synthetic construct
  misc_feature 1..36: note = Repeat Sequence
  source 1..36: mol_type = other RNA
gctgtgagtcacagtaaaaatgaaggtatatccaac

SEQ ID NO: 534 - RNA, 36 nt, synthetic construct
  misc_feature 1..36: note = Repeat Sequence
  source 1..36: mol_type = other RNA
gatgtatatgctatgattttgtatggtacatccaac

SEQ ID NO: 535 - RNA, 37 nt, synthetic construct
  misc_feature 1..37: note = Repeat Sequence
  source 1..37: mol_type = other RNA
gttgcagaacccgaatagacgaatgaaggaatgcaac

SEQ ID NO: 536 - RNA, 33 nt, synthetic construct
  misc_feature 1..33: note = Repeat Sequence
  source 1..33: mol_type = other RNA
gttgagctctgcattacgcagatgaatgacgag

SEQ ID NO: 537 - RNA, 32 nt, synthetic construct
  misc_feature 1..32: note = Repeat Sequence
  source 1..32: mol_type = other RNA
gatatatcttgtatgcatatgtaggttgtgag

SEQ ID NO: 538 - RNA, 33 nt, synthetic construct
  misc_feature 1..33: note = Repeat Sequence
  source 1..33: mol_type = other RNA
gttgcaacttacgcataggtgtaaaatacgagg

SEQ ID NO: 539 - RNA, 32 nt, synthetic construct
  misc_feature 1..32: note = Repeat Sequence
  source 1..32: mol_type = other RNA
gatgcaacttagatgcatatgtaagttgtgag

SEQ ID NO: 540 - RNA, 32 nt, synthetic construct
  misc_feature 1..32: note = Repeat Sequence
  source 1..32: mol_type = other RNA
gttgcaatgaacgtatgtgcatgaggtgtgag

SEQ ID NO: 541 - RNA, 32 nt, synthetic construct
  misc_feature 1..32: note = Repeat Sequence
  source 1..32: mol_type = other RNA
gttgcaattcgtatgcgcaggtaagtttcgag

SEQ ID NO: 542 - RNA, 17 nt, synthetic construct
  misc_feature 1..17: note = Repeat Sequence
  source 1..17: mol_type = other RNA
tgtgcatgaggtgtgag

SEQ ID NO: 543 - RNA, 32 nt, synthetic construct
  misc_feature 1..32: note = Repeat Sequence
  source 1..32: mol_type = other RNA
gttgcaatctgcgtacaggcgtaagatgtgag

SEQ ID NO: 544 - RNA, 17 nt, synthetic construct
  misc_feature 1..17: note = Repeat Sequence
  source 1..17: mol_type = other RNA
caggcgtaagatgtgag

SEQ ID NO: 545 - RNA, 32 nt, synthetic construct
  misc_feature 1..32: note = Repeat Sequence
  source 1..32: mol_type = other RNA
gatcatatctgcttgtatgggtatgctgcgag

SEQ ID NO: 546 - RNA, 17 nt, synthetic construct
  misc_feature 1..17: note = Repeat Sequence
  source 1..17: mol_type = other RNA
tatgggtatgctgcgag

SEQ ID NO: 547 - RNA, 32 nt, synthetic construct
  misc_feature 1..32: note = Repeat Sequence
  source 1..32: mol_type = other RNA
gttgcaacttacgcataggtgtaaaatacgag

SEQ ID NO: 548 - RNA, 32 nt, synthetic construct
  misc_feature 1..32: note = Repeat Sequence
  source 1..32: mol_type = other RNA
gattatatctgcttgtatgggtatactgcgag

SEQ ID NO: 549 - RNA, 38 nt, synthetic construct
  misc_feature 1..38: note = Repeat Sequence
  source 1..38: mol_type = other RNA
tcagctcacaacctacatatgcatacaagatatatcgt

SEQ ID NO: 550 - RNA, 17 nt, synthetic construct
  misc_feature 1..17: note = Repeat Sequence
  source 1..17: mol_type = other RNA
catatgtaggttgtgag

SEQ ID NO: 551 - RNA, 13 nt, synthetic construct
  misc_feature 1..13: note = Repeat Sequence
  source 1..13: mol_type = other RNA
tgtaggttgtgag

SEQ ID NO: 552 - RNA, 13 nt, synthetic construct
  misc_feature 1..13: note = Repeat Sequence
  source 1..13: mol_type = other RNA
catgaggtgtgag

SEQ ID NO: 553 - RNA, 36 nt, synthetic construct
  misc_feature 1..36: note = Repeat Sequence
  source 1..36: mol_type = other RNA
ggagagatctcaaacgattgctcgattagtcgagac

SEQ ID NO: 554 - RNA, 36 nt, synthetic construct
  misc_feature 1..36: note = Repeat Sequence
  source 1..36: mol_type = other RNA
gtcggaacgctcaacgattgcccctcacgaggggac

SEQ ID NO: 555 - RNA, 36 nt, synthetic construct
  misc_feature 1..36: note = Repeat Sequence
  source 1..36: mol_type = other RNA
accaaaacgactattgattgcccagtacgctgggac

SEQ ID NO: 556 - RNA, 37 nt, synthetic construct
  misc_feature 1..37: note = Repeat Sequence
  source 1..37: mol_type = other RNA
ggatccaatcctttttgattgcccaattcgttgggac

SEQ ID NO: 557 - RNA, 36 nt, synthetic construct
  misc_feature 1..36: note = Repeat Sequence
  source 1..36: mol_type = other RNA
ggatctgaggatcattattgctcgttacgacgagac

SEQ ID NO: 558 - RNA, 36 nt, synthetic construct
  misc_feature 1..36: note = Repeat Sequence
  source 1..36: mol_type = other RNA
gtctcagcgtactgagcaatcaaaaggtttcgcagg

SEQ ID NO: 559 - RNA, 36 nt, synthetic construct
  misc_feature 1..36: note = Repeat Sequence
  source 1..36: mol_type = other RNA
ctttcaagactaatagattgctccttacgaggagac

SEQ ID NO: 560 - skipped

SEQ ID NO: 561 - RNA, 36 nt, synthetic construct
  misc_feature 1..36: note = Repeat Sequence
  source 1..36: mol_type = other RNA
gtagaagacctcgctgattgctcggtgcgccgagac

SEQ ID NO: 562 - RNA, 36 nt, synthetic construct
  misc_feature 1..36: note = Repeat Sequence
  source 1..36: mol_type = other RNA
atggcaacagactctcattgcgcggtacgccgcgac

SEQ ID NO: 563 - RNA, 34 nt, synthetic construct
  misc_feature 1..34: note = Repeat Sequence
  source 1..34: mol_type = other RNA
gtcgctctctaacgcttgcccagtacgctgggac

SEQ ID NO: 564 - DNA, 36 bp, synthetic construct
  misc_feature 1..36: note = Repeat Sequence
  source 1..36: mol_type = other DNA
gctggaagactcaatgatggctccttacgaggagac

SEQ ID NO: 565 - RNA, 37 nt, synthetic construct
  misc_feature 1..37: note = Repeat Sequence
  source 1..37: mol_type = other RNA
ggttgaaccctcaacagattgctcggtaagccgagac

SEQ ID NO: 566 - RNA, 36 nt, synthetic construct
  misc_feature 1..36: note = Repeat Sequence
  source 1..36: mol_type = other RNA
cttgaaatcctgtcagattgctcccttcggggagac

SEQ ID NO: 567 - RNA, 36 nt, synthetic construct
  misc_feature 1..36: note = Repeat Sequence
  source 1..36: mol_type = other RNA
gctggaagactcaatgatggctccttacgaggagac

SEQ ID NO: 568 - RNA, 36 nt, synthetic construct
  misc_feature 1..36: note = Repeat Sequence
  source 1..36: mol_type = other RNA
ctaggaacgcacgcagattgctcggtacgccgagac

SEQ ID NO: 569 - RNA, 36 nt, synthetic construct
  misc_feature 1..36: note = Repeat Sequence
  source 1..36: mol_type = other RNA
attgcaacgcctaaagattgctcgatacgtcgagac

SEQ ID NO: 570 - RNA, 36 nt, synthetic construct
  misc_feature 1..36: note = Repeat Sequence
  source 1..36: mol_type = other RNA
gttcggcraycctttgattgctcagtacgctgagac

SEQ ID NO: 571 - RNA, 36 nt, synthetic construct
  misc_feature 1..36: note = Repeat Sequence
  source 1..36: mol_type = other RNA
gttgaacctagatcagatggctcagtacgctgagac

SEQ ID NO: 572 - RNA, 36 nt, synthetic construct
  misc_feature 1..36: note = Repeat Sequence
  source 1..36: mol_type = other RNA
ccctcaacacgtcagaaatgcccggcacgccgggac

SEQ ID NO: 573 - RNA, 36 nt, synthetic construct
  misc_feature 1..36: note = Repeat Sequence
  source 1..36: mol_type = other RNA
gtcgcaagactcgaataattgcccctctatggggac

SEQ ID NO: 574 - RNA, 37 nt, synthetic construct
  misc_feature 1..37: note = Repeat Sequence
  source 1..37: mol_type = other RNA
gctggggaccgatcctgattgctcgctgcggcgagac

SEQ ID NO: 575 - RNA, 36 nt, synthetic construct
  misc_feature 1..36: note = Repeat Sequence
  source 1..36: mol_type = other RNA
ctctcaatggataacgattgctctctacggagagac

SEQ ID NO: 576 - RNA, 36 nt, synthetic construct
  misc_feature 1..36: note = Repeat Sequence
  source 1..36: mol_type = other RNA
gttgaaccctcaacagattgctcggtaagccgagac

SEQ ID NO: 577 - DNA, 36 bp, synthetic construct
  misc_feature 1..36: note = Repeat Sequence
  source 1..36: mol_type = other DNA
gttgaaccctcaacagattgctcggtaagccgagac

SEQ ID NO: 578 - DNA, 36 bp, synthetic construct
  misc_feature 1..36: note = Repeat Sequence
  source 1..36: mol_type = other DNA
gtcgcaagactcgaataattgcccctctatggggac

SEQ ID NO: 579 - DNA, 36 bp, synthetic construct
  misc_feature 1..36: note = Repeat Sequence
  source 1..36: mol_type = other DNA
gtcggaacgctcaacgattgcccctcacgaggggac

SEQ ID NO: 580 - DNA, 36 bp, synthetic construct
  misc_feature 1..36: note = Repeat Sequence
  source 1..36: mol_type = other DNA
gttgaacctagatcagatggctcagtacgctgagac

SEQ ID NO: 581 - DNA, 36 bp, synthetic construct
  misc_feature 1..36: note = Repeat Sequence
  source 1..36: mol_type = other DNA
ctctcaatggataacgattgctctctacggagagac

SEQ ID NO: 582 - DNA, 36 bp, synthetic construct
  misc_feature 1..36: note = Repeat Sequence
  source 1..36: mol_type = other DNA
actgaaaccaccaacgattgcgctcctcggagcgac

SEQ ID NO: 583 - DNA, 36 bp, synthetic construct
  misc_feature 1..36: note = Repeat Sequence
  source 1..36: mol_type = other DNA
accaaaacgactattgattgcccagtacgctgggac

SEQ ID NO: 584 - RNA, 36 nt, synthetic construct
  misc_feature 1..36: note = Repeat Sequence
  source 1..36: mol_type = other RNA
actgaaaccaccaacgattgcgctcctcggagcgac

SEQ ID NO: 585 - DNA, 37 bp, synthetic construct
  misc_feature 1..37: note = Repeat Sequence
  source 1..37: mol_type = other DNA
ggttgaaccctcaacagattgctcggtaagccgagac

SEQ ID NO: 586 - RNA, 35 nt, synthetic construct
  misc_feature 1..35: note = Repeat Sequence
  source 1..35: mol_type = other RNA
gttagaatataaccctgtttgtaggggtaataaaa

SEQ ID NO: 587 - RNA, 37 nt, synthetic construct
  misc_feature 1..37: note = Repeat Sequence
  source 1..37: mol_type = other RNA
agttgtgtctacctcctttttgagaggtaaaaacaac

SEQ ID NO: 588 - RNA, 35 nt, synthetic construct
  misc_feature 1..35: note = Repeat Sequence
  source 1..35: mol_type = other RNA
atttgactatacccctatttgagaggggaataaaa

SEQ ID NO: 589 - RNA, 35 nt, synthetic construct
  misc_feature 1..35: note = Repeat Sequence
  source 1..35: mol_type = other RNA
attggaatataaccccgtttgtaggggtagtaaaa

SEQ ID NO: 590 - RNA, 35 nt, synthetic construct
  misc_feature 1..35: note = Repeat Sequence
  source 1..35: mol_type = other RNA
ttgactacactctctatctcttagggagactgaaa

SEQ ID NO: 591 - RNA, 35 nt, synthetic construct
  misc_feature 1..35: note = Repeat Sequence
  source 1..35: mol_type = other RNA
gtttgaatataaccctgtttgtaggggtaataaaa

SEQ ID NO: 592 - RNA, 35 nt, synthetic construct
  misc_feature 1..35: note = Repeat Sequence
  source 1..35: mol_type = other RNA
gatagaatataaccctgtttgtaggggtaataaaa

SEQ ID NO: 593 - RNA, 37 nt, synthetic construct
  misc_feature 1..37: note = Repeat Sequence
  source 1..37: mol_type = other RNA
agtcagactataccctcgtttgtagggggaataaaac

SEQ ID NO: 594 - RNA, 36 nt, synthetic construct
  misc_feature 1..36: note = Repeat Sequence
  source 1..36: mol_type = other RNA
gttgcatctgcctgctgtttgcaaggtaaaaacaac

SEQ ID NO: 595 - RNA, 37 nt, synthetic construct
  misc_feature 1..37: note = Repeat Sequence
  source 1..37: mol_type = other RNA
gatttagactaccccaaaaacgaaggggactaaaacc

SEQ ID NO: 596 - RNA, 36 nt, synthetic construct
  misc_feature 1..36: note = Repeat Sequence
  source 1..36: mol_type = other RNA
gttttagtccccttcgtttttggggtagtctaaatc

SEQ ID NO: 597 - RNA, 36 nt, synthetic construct
  misc_feature 1..36: note = Repeat Sequence
  source 1..36: mol_type = other RNA
gttgtaactgcccttattttgaagggtaaacacaac

SEQ ID NO: 598 - RNA, 37 nt, synthetic construct
  misc_feature 1..37: note = Repeat Sequence
  source 1..37: mol_type = other RNA
ggttagaatataaccctgtttgtaggggtaataaaac

SEQ ID NO: 599 - RNA, 17 nt, synthetic construct
  misc_feature 1..17: note = Repeat Sequence
  source 1..17: mol_type = other RNA
agaagaaggattgggac

SEQ ID NO: 600 - RNA, 17 nt, synthetic construct
  misc_feature 1..17: note = Repeat Sequence
  source 1..17: mol_type = other RNA
aaaacaaggattgaaac

SEQ ID NO: 601 - RNA, 17 nt, synthetic construct
  misc_feature 1..17: note = Repeat Sequence
  source 1..17: mol_type = other RNA
cgtacgtggattgaaac

SEQ ID NO: 602 - RNA, 37 nt, synthetic construct
  misc_feature 1..37: note = Repeat Sequence
  source 1..37: mol_type = other RNA
gtctgaaagctacttccaataaaacaaggattgaaac

SEQ ID NO: 603 - RNA, 37 nt, synthetic construct
  misc_feature 1..37: note = Repeat Sequence
  source 1..37: mol_type = other RNA
cttgcaggccttgaatagaggagttaaggaatgcaac

SEQ ID NO: 604 - RNA, 17 nt, synthetic construct
  misc_feature 1..17: note = Repeat Sequence
  source 1..17: mol_type = other RNA
gaatgaaggaatgcaac

SEQ ID NO: 605 - RNA, 37 nt, synthetic construct
  misc_feature 1..37: note = Repeat Sequence
  source 1..37: mol_type = other RNA
ctttcatactcagaacaaagggattaaggaatgcaac

SEQ ID NO: 606 - RNA, 37 nt, synthetic construct
  misc_feature 1..37: note = Repeat Sequence
  source 1..37: mol_type = other RNA
atttcatactcagaacaaagggattaaggaatgcaac

SEQ ID NO: 607 - RNA, 37 nt, synthetic construct
  misc_feature 1..37: note = Repeat Sequence
  source 1..37: mol_type = other RNA
gttgcgcgaatagaataaaggaattaaggaatgcaac

SEQ ID NO: 608 - RNA, 28 nt, synthetic construct
  misc_feature 1..28: note = Repeat Sequence
  source 1..28: mol_type = other RNA
gctagcccgtgcgcgcagggacgagtgg

SEQ ID NO: 609 - RNA, 20 nt, synthetic construct
  misc_feature 1..20: note = Repeat Sequence
  source 1..20: mol_type = other RNA
gaatttctactattgtagat

SEQ ID NO: 610 - RNA, 20 nt, synthetic construct
  misc_feature 1..20: note = Repeat Sequence
  source 1..20: mol_type = other RNA
agatttctacttttgtagat

SEQ ID NO: 611 - RNA, 13 nt, synthetic construct
  misc_feature 1..13: note = Repeat Sequence
  source 1..13: mol_type = other RNA
aggtacatccaac

SEQ ID NO: 612 - RNA, 17 nt, synthetic construct
  misc_feature 1..17: note = Repeat Sequence
  source 1..17: mol_type = other RNA
tgcggtgtaattcgagg

SEQ ID NO: 613 - RNA, 36 nt, synthetic construct
  misc_feature 1..36: note = Repeat Sequence
  source 1..36: mol_type = other RNA
gatgtgaacgacctttttttgcggtgtgcttcgagg

SEQ ID NO: 614 - RNA, 36 nt, synthetic construct
  misc_feature 1..36: note = Repeat Sequence
  source 1..36: mol_type = other RNA
ggtggatatcatcttaaaaagtgaggtacatccaac

SEQ ID NO: 615 - RNA, 36 nt, synthetic construct
  misc_feature 1..36: note = Repeat Sequence
  source 1..36: mol_type = other RNA
ggtgtgaacgacctttttttgcggtgtaattcgagg

SEQ ID NO: 616 - RNA, 18 nt, synthetic construct
  misc_feature 1..18: note = Repeat Seqeunce
  source 1..18: mol_type = other RNA
ttgcggtgtacttcgagg

SEQ ID NO: 617 - RNA, 68 nt, synthetic construct
  misc_feature 1..68: note = Repeat Sequence
  source 1..68: mol_type = other RNA
gatatatcttgtatgcatatgtaggttgtgagaatgtgaacgaccttcttttgcggtgtacttcgagg

SEQ ID NO: 618 - RNA, 14 nt, synthetic construct
  misc_feature 1..14: note = Repeat Sequence
  source 1..14: mol_type = other RNA
aaggttgatacagc

SEQ ID NO: 619 - RNA, 36 nt, synthetic construct
  misc_feature 1..36: note = Repeat Sequence
  source 1..36: mol_type = other RNA
gctgtaagtcatggaaaaatggtgagtacatccaac

SEQ ID NO: 620 - RNA, 18 nt, synthetic construct
  misc_feature 1..18: note = Repeat Sequence
  source 1..18: mol_type = other RNA
atggtgagtacatccaac

SEQ ID NO: 621 - RNA, 13 nt, synthetic construct
  misc_feature 1..13: note = Repeat Sequence
  source 1..13: mol_type = other RNA
gagcacatccaac

SEQ ID NO: 622 - RNA, 13 nt, synthetic construct
  misc_feature 1..13: note = Repeat Sequence
  source 1..13: mol_type = other RNA
gggtacatccaac

SEQ ID NO: 623 - RNA, 36 nt, synthetic construct
  misc_feature 1..36: note = Repeat Sequence
  source 1..36: mol_type = other RNA
gttgcgtttgcccgtgatttcgggtgtgtatacagg

SEQ ID NO: 624 - RNA, 13 nt, synthetic construct
  misc_feature 1..13: note = Repeat Sequence
  source 1..13: mol_type = other RNA
aggtatatccaac

SEQ ID NO: 625 - RNA, 36 nt, synthetic construct
  misc_feature 1..36: note = Repeat Sequence
  source 1..36: mol_type = other RNA
ggcgtatgtctacctgaaaaagaaggtatatccaac

SEQ ID NO: 626 - RNA, 13 nt, synthetic construct
  misc_feature 1..13: note = Repeat Sequence
  source 1..13: mol_type = other RNA
ggctacatacagc

SEQ ID NO: 627 - RNA, 36 nt, synthetic construct
  misc_feature 1..36: note = Repeat Sequence
  source 1..36: mol_type = other RNA
ggtgtatgtgcaccatatatgtaggtgacatacagc

SEQ ID NO: 628 - RNA, 36 nt, synthetic construct
  misc_feature 1..36: note = Repeat Sequence
  source 1..36: mol_type = other RNA
gatgtgaacgacctttttttgcggtgtacttcgagg

SEQ ID NO: 629 - RNA, 13 nt, synthetic construct
  misc_feature 1..13: note = Repeat Sequence
  source 1..13: mol_type = other RNA
gtgtacttcgagg

SEQ ID NO: 630 - RNA, 36 nt, synthetic construct
  misc_feature 1..36: note = Repeat Seqeunce
  source 1..36: mol_type = other RNA
gatgtaaatcatctataaaagaaaggtacatccaac

SEQ ID NO: 631 - RNA, 12 nt, synthetic construct
  misc_feature 1..12: note = Repeat Sequence
  source 1..12: mol_type = other RNA
ggtacatccaac

SEQ ID NO: 632 - RNA, 36 nt, synthetic construct
  misc_feature 1..36: note = Repeat Sequence
  source 1..36: mol_type = other RNA
gctgcactgcaccgcccattgatggtgtgctctagg

SEQ ID NO: 633 - RNA, 36 nt, synthetic construct
  misc_feature 1..36: note = Repeat Sequence
  source 1..36: mol_type = other RNA
attgtaggcgacctttttttgcgatgtagttcgagg

SEQ ID NO: 634 - RNA, 13 nt, synthetic construct
  misc_feature 1..13: note = Repeat Sequence
  source 1..13: mol_type = other RNA
atgtagttcgagg

SEQ ID NO: 635 - RNA, 36 nt, synthetic construct
  misc_feature 1..36: note = Repeat Sequence
  source 1..36: mol_type = other RNA
agtgtatgattacctgtagtatgaggtacatccaac

SEQ ID NO: 636 - RNA, 36 nt, synthetic construct
  misc_feature 1..36: note = Repeat Sequence
  source 1..36: mol_type = other RNA
gctgcaagagctcctaatttgaggggtgcatacagg

SEQ ID NO: 637 - RNA, 36 nt, synthetic construct
  misc_feature 1..36: note = Repeat Sequence
  source 1..36: mol_type = other RNA
gatagttttaacttccatttgaaatgtaaatgcaac

SEQ ID NO: 638 - RNA, 13 nt, synthetic construct
  misc_feature 1..13: note = Repeat Sequence
  source 1..13: mol_type = other RNA
atgtaaatgcaac

SEQ ID NO: 639 - RNA, 36 nt, synthetic construct
  misc_feature 1..36: note = Repeat Sequence
  source 1..36: mol_type = other RNA
gttgtaagagacccgaattttagctgtgtatacagg

SEQ ID NO: 640 - RNA, 36 nt, synthetic construct
  misc_feature 1..36: note = Repeat Sequence
  source 1..36: mol_type = other RNA
gttgtgaatgcaggcatttttgatggtaaatccaac

SEQ ID NO: 641-646 - skipped (6 entries)

(19) United States
(12) Patent Application Publication
(10) Pub. No.: US 2024/0218393 A1
(43) Pub. Date: Jul. 4, 2024

(54) VECTORS ENCODING GENE EDITING SYSTEMS AND USES THEREOF

(71) Applicant: Mammoth Biosciences, Inc., Brisbane, CA (US)

(72) Inventors: Pei-Qi LIU, Oakland, CA (US); Wiputra Jaya HARTONO, San Francisco, CA (US); Lucas Benjamin HARRINGTON, San Francisco, CA (US); Sean CHEN, Brisbane, CA (US)

(21) Appl. No.: 18/413,896

(22) Filed: Jan. 16, 2024

Related U.S. Application Data
(63) Continuation of application No. PCT/US22/74074, filed on Jul. 22, 2022.
(60) Provisional application No. 63/225,210, filed on Jul. 23, 2021.
(60) Provisional application No. 63/283,100, filed on Nov. 24, 2021.
(60) Provisional application No. 63/335,467, filed on Apr. 27, 2022.
(60) Provisional application No. 63/342,584, filed on May 16, 2022.

(51) Int. Cl. C12N 15/86; C12N 15/11

(52) U.S. Cl. CPC C12N 15/86; C12N 15/111; C12N 2310/20; C12N 2750/14143; C12N 2830/008; C12N 2830/48

(57) ABSTRACT

Provided herein are viral vectors comprising a transgene encoding a compact effector protein and uses thereof. These effector proteins are shown to be active with guide RNAs and may be characterized as CRISPR-associated (Cas) proteins. Due to the small nature of the effector protein, the viral vector has ample room for additional genome editing cargo, such as a guide nucleic acid and a donor nucleic acid. Various compositions, systems, and methods of the present disclosure leverage the activities of these effector proteins for the modification, detection, and engineering of nucleic acids.

### CROSS REFERENCE TO RELATED APPLICATIONS

This application is a continuation of International Patent Application No. PCT/US22/74074, filed Jul. 22, 2022, which claims the benefit of U.S. Provisional Application No. 63/225,210, filed Jul. 23, 2021, U.S. Provisional Application No. 63/283,100, filed Nov. 24, 2021, U.S. Provisional Application No. 63/335,467, filed Apr. 27, 2022, and U.S. Provisional Application No. 63/342,584, filed May 16, 2022, the disclosures of which are incorporated herein by reference in their entirety.

### SEQUENCE LISTING

The instant application contains a Sequence Listing, which has been submitted via Patent Center. The Sequence Listing titled 203477-708301US_SL.xml, which was created on Jan. 12, 2024, and is 817,654 bytes in size, is hereby incorporated by reference in its entirety.

### BACKGROUND

Clustered regularly interspaced short palindromic repeats (CRISPR) is a segment of DNA found in the genomes of certain prokaryotic organisms, including some bacteria and archaea, that includes repeated short sequences of nucleotides interspersed at regular intervals between unique sequences of nucleotides derived from another organism. Programmable nucleases are proteins that bind and cleave nucleic acids in a sequence-specific manner. A programmable nuclease, such as a CRISPR-associated (Cas) protein, may be coupled to a guide nucleic acid that imparts activity or sequence selectivity to the programmable nuclease. The programmable nuclease and guide nucleic acid may form a complex that recognizes a target region of a nucleic acid and cleaves the nucleic acid within the target region or at a position near or adjacent to the target region. In general, guide nucleic acids comprise a CRISPR RNA (crRNA), or a component thereof, that is at least partially complementary to a target nucleic acid. In some cases, guide nucleic acids comprise a trans-activating crRNA (tracrRNA), at least a portion of which interacts with the programmable nuclease. In some cases, a tracrRNA is provided separately from the crRNA. The tracrRNA may hybridize to a portion of the crRNA that does not hybridize to the target nucleic acid. In some cases, a guide nucleic acid comprises a single guide RNA (sgRNA). An sgRNA may comprise a first region linked to a second region, wherein the first region interacts with the programmable nuclease, and the second region hybridizes with the target nucleic acid. In some cases, a sgRNA does not comprise a tracrRNA.

Programmable nucleases may cleave nucleic acids, including single stranded RNA (ssRNA), double stranded DNA (dsDNA), and single-stranded DNA (ssDNA). Programmable nucleases may provide cis cleavage activity, trans cleavage activity, nickase activity, or a combination thereof. Cis cleavage activity is cleavage of a target nucleic acid, a region of which is hybridized to a guide RNA (e.g. a crRNA or a sgRNA), wherein cleavage occurs near, within or directly adjacent to the region of the target nucleic acid that is hybridized to guide nucleic acid. Trans cleavage activity (also referred to as transcollateral cleavage) is cleavage of ssDNA or ssRNA that is near, but not hybridized to the guide RNA. Trans cleavage activity may be triggered by the hybridization of a guide RNA to the target nucleic acid. Nickase activity comprises the selective cleavage of one strand of a dsDNA molecule.

In order for effector proteins to be used therapeutically, e.g., for genome editing, they must be packaged in an appropriate manner to be delivered to a subject. In some instances, that may include genetically modifying a cell that will be delivered to the subject. Adeno-associated virus (AAV) vectors are useful delivery platforms for therapeutic genome editing. However, if the AAV vector is loaded with too much cargo (e.g., a transgene encoding genome editing components totaling more than 4.5 kb in length), viral production becomes compromised. For example, if the transgene included a region encoding a Cas9 protein, which is ˜4 kb, a guide nucleic acid, and respective promoters, there would be no substantial space remaining for a donor nucleic acid.

### SUMMARY

Disclosed herein are compositions comprising viral vectors encoding an effector protein and a guide nucleic acid. Often the viral vector is an AAV vector. The guide nucleic acid may also be referred to as a guide RNA, and the effector protein may be considered an RNA guided nuclease. Often, the effector protein is a Cas effector protein. The effector protein may share substantial sequence identity (e.g., >50%) with a Cas effector protein. The effector protein may comprise more than 200 contiguous amino acids that are at least 75% identical to a Cas effector protein. The Cas effector protein is generally a small Cas effector protein, e.g., less than 900 amino acids in length, allowing for the inclusion of other gene editing components in the viral vector, such as a guide nucleic acid, donor nucleic acid, and respective promoters, without compromising production of the virus. In some instances, the portion of the AAV vector encoding the genome editing components is less than 5 kb. In some instances, the portion of the AAV vector encoding the genome editing components is between 4 kb and 5 kb. In some instances, the AAV vector is AAV8 vector.

In some instances, the AAV vector is a self-complementary AAV (scAAV) vector. The coding region of the scAAV vector forms an intramolecular double-stranded DNA template in contrast to a standard AAV vector having a single-stranded DNA template. The two complementary sequences of the scAAV coding region associate to form dsDNA that is ready for expression. An scAAV vector does not require cell mediated synthesis of a second strand like the standard AAV vector. Consequently, the scAAV vector provides greater and longer expression of the genome editing components (also referred to collectively as the transgene) that it carries relative to the standard AAV vector. Whereas an AAV carrying an AAV vector can be efficiently produced with a standard AAV vector carrying genome editing components having a total length of about 4 kb to about 5 kb, the genome editing components of an scAAV vector has a total length of about 2 kb to about 3 kb. Described herein are many compact Cas effector proteins that are suitable for scAAV. Due to their small size, there is space for a guide nucleic acid and a donor nucleic acid. In some instances, the length of the Cas effector protein encoded by the scAAV vector transgene is less than 500 amino acids. In addition to the efficient viral production and expression afforded by scAAV, scAAV can offer the advantage of a lower dose or lower viral titer than a standard AAV vector to sufficiently infect cells. A viral titer or dose that is too high can be toxic to cells. This is particularly useful for cells that are notoriously difficult to infect, e.g., muscle cells.

In some instances, the AAV vector comprises a donor nucleic acid. The donor nucleic acid may comprise a cDNA encoding a wildtype or mutant protein. The donor nucleic acid may comprise a regulatory element, such as a promoter or transcription factor binding site. In some instances, the donor nucleic acid may encode a fusion protein. In some instances, the AAV vector comprises a sequence that encodes a chimeric antigen receptor (CAR). CARs are engineered proteins that combine an antigen-binding peptide with a portion of a T cell receptor, such that when the antigen binding peptide binds its respective antigen, a T cell expressing the CAR is activated. Often CARs are engineered to bind antigens expressed by cancer cells, e.g., CD19 and BCMA. Thus, the instant disclosure provides for CAR T-cells, methods of quickly and efficiently producing CAR T-cells, and methods of administering such CAR T-cells to patients in need thereof. Due to the small size of the Cas effector proteins encoded by the AAV vectors, the AAV vectors have sufficient space for a construct encoding the CAR. In some instances, the genome editing components include a guide RNA or another nucleic acid for knocking out a Human Leukocyte Antigen (HLA), rendering the T cell immunologically compatible (“allogeneic”) with a patient that lacks that HLA, which is especially advantageous for patients that do not have enough T cells, or do not have T cells that replicate well enough ex vivo, to produce autologous CAR T-cells. By combining nucleic acids encoding a Cas effector protein, HLA directed guide RNA, and CAR into one viral vector, the process of producing CAR T-cells, whether autologous or allogeneic, is accelerated and produces a higher quantity and quality of CAR T-cells, relative to current methods that require multiple viral vectors and/or electroporation.

### Certain Embodiments

Disclosed herein, in some aspects, are compositions comprising a self-complementary adeno-associated viral (scAAV) vector, wherein the scAAV vector comprises a transgene comprising: a first region that encodes an effector protein; and a second region that encodes a guide nucleic acid, wherein at least a portion of the guide nucleic acid is complementary to a target region of a target nucleic acid. Also disclosed herein, in some aspects are compositions comprising a AAV8 vector, wherein the AAV8 vector comprises a transgene comprising: a first region that encodes an effector protein; a second region that encodes a guide nucleic acid, wherein at least a portion of the guide nucleic acid is complementary to a target region of a target nucleic acid. Also disclosed herein, in some aspects are compositions comprising the scAAV vector or the AAV8 vector further comprises a third region comprising a donor nucleic acid. Also disclosed herein, in some aspects are compositions comprising a viral vector, wherein the viral vector comprises a transgene comprising: a first region that encodes an effector protein; a second region that encodes a guide nucleic acid, wherein at least a portion of the guide nucleic acid is complementary to a target region of a target nucleic acid; and a third region that comprises a donor nucleic acid. In some instances, the viral vector is an adeno-associated viral (AAV) vector. In some instances, the length of the effector protein is less than about 1000, less than about 900, less than about 800, less than about 700, less than about 600, less than about 500, less than about 450 amino acids, or less than about 400 amino acids. In some instances, the length of the effector protein is at least about 300, at least about 350, at least about 400, or at least about 450 linked amino acids. In some instances, the length of the effector protein is about 300 to about 800 linked amino acids. In some instances, the length of the effector protein is about 400 to about 600 linked amino acids. In some instances, the length of the effector protein is about 420 to about 480 linked amino acids. In some instances, the effector protein is a Type V Cas effector protein. In some instances, the effector protein is a Type VI Cas effector protein. In some instances, the effector protein is a Cas12 protein. In some instances, the effector protein is a Cas13 protein. In some instances, the effector protein is a Cas14 protein. In some instances, the effector protein is at least about 70%, at least about 75%, at least about 80%, at least about 85%, at least about 90%, at least about 95%, at least about 98%, at least about 99%, or at least 100% identical to any one of the sequences recited in TABLE 1. In some instances, the sequence of the first region encoding the effector protein is at least about 70%, at least about 75%, at least about 80%, at least about 85%, at least about 90%, at least about 95%, at least about 98%, at least about 99%, or at least 100% identical to SEQ ID NO: 23. In some embodiments, the guide nucleic acid comprises a repeat sequence selected from TABLE 3. In some instances, the guide RNA comprises a crRNA. In some embodiments, a tracrRNA is linked to the crRNA. In some embodiments, the guide RNA comprises a sgRNA. In some embodiments, the sgRNA comprises at least a portion of or all of a repeat sequence, at least a portion of or all of a tracrRNA sequence, or a combination thereof. In some embodiments, the guide nucleic acid is at least about 80%, at least about 85%, at least about 90%, at least about 95%, at least about 98%, at least about 99% or 100% identical to any one of the sequences recited in TABLE 3, TABLE 4, TABLE 6, or TABLE 7. In some instances, the guide nucleic acid is at least about 80%, at least about 85%, at least about 90%, at least about 95%, at least about 98%, at least about 99% or 100% identical to a sequence selected from SEQ ID NOS: 202-207 and 329-367. In some instances, the guide nucleic acid comprises a sequence at least about 80%, at least about 85%, at least about 90%, at least about 95%, at least about 98%, at least about 99% or 100% identical to any one of SEQ ID NO: 214 and 231-232. In some instances, the guide nucleic acid comprises a sequence at least about 80%, at least about 85%, at least about 90%, at least about 95%, at least about 98%, at least about 99% or 100% identical to SEQ ID NO: 215. In some instances, the guide nucleic acid comprises a sequence with at least 8, at least 9, at least 10, at least 11, at least 12, or at least 13 contiguous nucleotides of SEQ ID NO: 215. In some embodiments, either the 5′ or 3′ end of the target region is adjacent to a protospacer adjacent motif (PAM) of TABLE 1.1. In some embodiments, the effector protein comprises a sequence that is at least 95% identical to a sequence of TABLE 1, wherein the guide nucleic acid comprises a repeat sequence that is at least 95% identical to a sequence of TABLE 3 and corresponds to the effector protein as shown in TABLE 3, and wherein either the 5′ or 3′ end of the target region is within 20 nucleotides of a PAM of TABLE 1.1 and corresponds to the effector protein as shown in TABLE 1.1. In some instances, the length of the donor nucleic acid is less than about 2000, less than about 1950, less than about 1900, less than about 1850, less than about 1800, less than about 1750, less than about 1700, less than about 1650, less than about 1600, less than about 1550, less than about 1500, less than about 1450, less than about 1400, less than about 1350, less than about 1300, less than about 1250, less than about 1200, less than about 1150, less than about 1100, less than about 1050, or less than about 1000 linked nucleosides. In some instances, the length of the donor nucleic acid is at least about 10, at least about 20, at least about 30, at least about 40, at least about 50 linked nucleosides. In some instances, the length of the donor nucleic acid is about 50 to about 1500, about 800 to about 1200, about 50 to about 600, about 1000 to about 1200, about 1200 to about 1400, about 1400 to about 1600, about 1600 to about 1800, or about 1800 to about 2000, or about 500 to about 1500 linked nucleosides. In some instances, the length of the donor nucleic acid is about 800 to about 1200 linked nucleosides. In some embodiments, the donor nucleic acid comprises a cDNA. In some embodiments, the donor nucleic acid comprises a regulatory element or a primer binding site. In some embodiments, the donor nucleic acid encodes a mRNA, miRNA, siRNA, protein tag, detectable marker, or a combination thereof. In some instances, the donor nucleic acid encodes a chimeric antigen receptor or a portion thereof. In some instances, the target region of the target nucleic acid is at least 90%, at least 95% or 100% identical to an equal length portion of a gene encoding a human leukocyte antigen (HLA). In some instances, the target nucleic acid is a PCSK9 gene. In some embodiments, the composition described herein comprises a fourth region that encodes a second guide nucleic acid, wherein at least a portion of the guide nucleic acid is complementary to a second target region of the target nucleic acid. In some instances, the viral vector, scAAV vector or AAV8 vector comprises a first promoter that drives expression of the guide nucleic acid and the second promoter that drives expression of the effector protein. In some embodiments, the viral vector, scAAV vector or AAV8 vector comprises a first promoter that drives expression of the guide nucleic acid, the second promoter that drives expression of the effector protein, and a third promoter that drives expression of the second guide nucleic acid. In some embodiments, one or more of the first promoter, the second promoter, and the third promoter are same. In some embodiments, one or more of the first promoter, the second promoter, and the third promoter are different. In some embodiments, the viral vector, scAAV vector or AAV8 vector comprises a promoter selected from an EFS promoter, a U6 promoter, an H1 promoter, a 7SK promoter or any combination thereof. In some instances, the first promoter is a U6 promoter or H1 promoter. In some instances, the second promoter is an EFS promoter. In some embodiments, the viral vector, the scAAV vector or the AAV8 vector is a self-inactivating viral vector.

In some instances, AAV vectors comprise a transgene, wherein the transgene comprises or consists essentially of a nucleic acid encoding a effector protein, a guide nucleic acid, a first promoter driving expression of the effector protein, a second promoter driving expression of the guide nucleic acid, wherein the amino acid sequence of the effector protein is at least about 75%, at least about 80%, at least about 85%, at least about 90%, at least about 95%, at least about 98%, at least about 99% or 100% identical to a sequence selected from any one of sequences recited in TABLE 1. In some instances, the composition further comprises a donor nucleic acid. In some instances, the length of the donor nucleic acid is about 50 bp to about 600 bp. In some instances, the amino acid sequence of the effector protein is at least about 75%, at least about 80%, at least about 85%, at least about 90%, at least about 95%, at least about 98%, at least about 99% or 100% identical to a sequence selected from SEQ ID NOS: 46-52, 54-58, 62-84, 87-89, 93-104, 113, 130, 131, 145, and 150. In some instances, the length of the donor nucleic acid is about 1 kb to about 1.2 kb, about 1.2 kb to about 1.4 kb, about 1.4 kb to about 1.6 kb, about 1.6 kb to about 1.8 kb, or about 1.8 kb to about 2 kb. In some instances, wherein the amino acid sequence of the effector protein is at least about 75%, at least about 80%, at least about 85%, at least about 90%, at least about 95%, at least about 98%, at least about 99% or 100% identical to a sequence selected from SEQ ID NOS: 1-45, 53, 85, 86, 90-92, 105-112, 114-129, 132-144, 146-149, and 151-156. In some instances, the donor nucleic acid encodes a chimeric antigen receptor. In some instances, the AAV vector is AAV8 vector. In some instances, wherein the amino acid sequence of the effector protein is at least about 75%, at least about 80%, at least about 85%, at least about 90%, at least about 95%, at least about 98%, at least about 99% or 100% identical to a sequence selected from SEQ ID NOS: 1-45, 53, 85, 86, 90-92, 105-112, 114-129, 132-144, 146-149, and 151-156. In some instances, the amino acid sequence of the effector protein is at least about 75%, at least about 80%, at least about 85%, at least about 90%, at least about 95%, at least about 98%, at least about 99% or 100% identical to SEQ ID NO: 23. In some instances, the length of the guide RNA is less than about 200 linked nucleosides. In some instances, the length of the crRNA is less than about 100, less than about 80, or less than about 60 linked nucleosides. In some instances, the first promoter is an EFS promoter and/or the second promoter is a U6 promoter or an H1 promoter. In some embodiments, the vector further comprises a fourth region comprising a third promoter that drives expression of a second guide nucleic acid, and a nucleotide sequence encoding the second guide nucleic acid. In some embodiments, the third promoter is a 7SK promoter. In some instances, the guide nucleic acid comprises or consists essentially of a crRNA. In some embodiments, the guide nucleic acid comprises or consists essentially of a sgRNA. In some embodiments, the sgRNA comprises a handle sequence and a spacer sequence. In some embodiments, the handle sequence comprises one or more of a linker, at least a portion of or all of a repeat sequence, and at least a portion of or all of a tracrRNA sequence. In some instances, the viral vector or the scAAV is a self-inactivating viral vector.

Disclosed herein, in some aspects, are compositions comprising an scAAV vector comprising a transgene, wherein the transgene comprises or consists essentially of a nucleic acid encoding a effector protein, a guide nucleic acid, a first promoter driving expression of the effector protein, and a second promoter driving expression of the guide nucleic acid, wherein the amino acid sequence of the effector protein is at least about 75%, at least about 80%, at least about 85%, at least about 90%, at least about 95%, at least about 98%, at least about 99% or 100% identical to a sequence selected from SEQ ID NOS: 1-45, 53, 85, 86, 90-92, 105-112, 114-129, 132-144, 146-149, and 151-156. In some instances, the amino acid sequence of the effector protein is at least about 75%, at least about 80%, at least about 85%, at least about 90%, at least about 95%, at least about 98%, at least about 99% or 100% identical to SEQ ID NO: 23. In some instances, the length of the guide RNA is less than about 200 linked nucleosides. In some instances, the length of the crRNA is less than about 100, less than about 80, or less than about 60 linked nucleosides. In some instances, the first promoter is an EFS promoter and/or the second promoter is a U6 promoter or an H1 promoter. In some embodiments, the vector further comprises a fourth region comprising a third promoter that drives expression of a second guide nucleic acid, and a nucleotide sequence encoding the second guide nucleic acid. In some embodiments, the third promoter is a 7SK promoter. In some instances, the guide nucleic acid comprises or consists essentially of a crRNA. In some embodiments, the guide nucleic acid comprises or consists essentially of a sgRNA. In some embodiments, the sgRNA comprises a handle sequence and a spacer sequence. In some embodiments, the handle sequence comprises at least a portion of or all of a repeat sequence and at least a portion of or all of a tracrRNA sequence. In some instances, the amino acid sequence of the effector protein is at least about 75%, at least about 80%, at least about 85%, at least about 90%, at least about 95%, at least about 98%, at least about 99% or 100% identical to SEQ ID NO: 23. In some instances, the viral vector or the scAAV is a self-inactivating viral vector.

Disclosed herein, in some aspects, are compositions comprising an adeno-associated virus comprising a composition disclosed herein. Also disclosed herein, in some aspects, are pharmaceutical compositions, comprising a composition disclosed herein or an adeno-associated virus disclosed herein, and a pharmaceutically acceptable excipient. Also disclosed herein, in some aspects, are methods of modifying a target nucleic acid, the methods comprising contacting the target nucleic acid with a composition disclosed herein, an adeno-associated virus disclosed herein, or a pharmaceutical composition disclosed herein, thereby modifying the target nucleic acid. In some instances, contacting occurs in vitro. In some instances, contacting occurs in vivo. In some instances, contacting occurs in a cell. In some instances, the cell is a eukaryotic cell. In some instances, the cell is a mammalian cell. In some instances, the cell is a human cell. In some instances, the cell is a liver cell or a hepatocyte. In some instances, the cell is selected from a T cell, an iPSC cell, a hepatocyte, a lung epithelial cell, a macrophage, a monocyte, a Kupffer cell, a microglial cell, a neutrophil, an eosinophil, a basophil, a mast cell, a dendritic cell, a natural killer cell, a B cell, and a CD34 positive hematopoietic stem cell. In some instances, contacting occurs ex vivo.

Disclosed herein, in some aspects are cells that has been contacted by a compositions described herein. Disclosed herein, in some aspects are systems comprising a cell and any one of the compositions described herein. In some instances, the cell has been contacted with the composition. In some instances, the cell is a eukaryotic cell. In some instances, the cell is a mammalian cell. In some instances, the cell is a human cell. In some instances, the cell is selected from a T cell, an iPSC cell, a hepatocyte, a lung epithelial cell, a macrophage, a monocyte, a Kupffer cell, a microglial cell, a neutrophil, an eosinophil, a basophil, a mast cell, a dendritic cell, a natural killer cell, a B cell, and a CD34 positive hematopoietic stem cell. In some instances, the cell is a T cell. Also disclosed herein, in some aspects, are methods of treating a disease comprising administering to a subject in need thereof a composition disclosed herein, an adeno-associated virus disclosed herein, a pharmaceutical composition disclosed herein, or a system disclosed herein.

Also disclosed herein are methods of modifying a cell comprising contacting the cell with a composition disclosed herein. In some instances, the cell is a eukaryotic cell; optionally wherein the eukaryotic cell is a mammalian cell; optionally wherein the mammalian cell is a human cell. In some instances, the cell is selected from a T cell, an iPSC cell, a hepatocyte, a lung epithelial cell, a macrophage, a monocyte, a Kupffer cell, a microglial cell, a neutrophil, an eosinophil, a basophil, a mast cell, a dendritic cell, a natural killer cell, a B cell, and a CD34 positive hematopoietic stem cell.

Disclosed herein, in some aspects are methods of producing an allogeneic CAR T-cell, the methods comprising contacting a population of T cells from a donor subject with an adeno-associated virus comprising a viral vector, wherein the viral vector comprises a transgene comprising: a first region that encodes an effector protein; a second region that encodes a guide nucleic acid, wherein at least a portion of the guide nucleic acid is complementary to a target region of a target nucleic acid; and a third region that comprises a donor nucleic acid. In some instances, the methods comprise culturing the T cells in vitro for less than two weeks. Also disclosed herein, in some aspects, are methods comprising: contacting a population of T cells from a first subject with an adeno-associated virus comprising a viral vector, wherein the viral vector comprises a transgene comprising: a first region that encodes an effector protein; a second region that encodes a guide nucleic acid, wherein at least a portion of the guide nucleic acid is complementary to a target region of a target nucleic acid; and a third region that comprises a donor nucleic acid to produce CAR T-cells; culturing the CAR T-cells in vitro for less than two weeks after contacting; and administering the CAR-T cells to a second subject. In some instances, the donor nucleic acid encodes a chimeric antigen receptor or a portion thereof. In some instances, the target region of the target nucleic acid is at least 90%, at least 95% or 100% identical to an equal length portion of a gene encoding a human leukocyte antigen (HLA).

Disclosed herein, in some aspects are scAAV vector comprising a transgene. In some embodiments, the transgene encodes an effector protein, a guide nucleic acid, and a chimeric antigen receptor. In some embodiments, the effector protein is a Cas protein. In some embodiments, the length of the Cas effector protein is 300 to 600 amino acids. In some embodiments, the guide nucleic acid comprises a spacer sequence that is at least 95% complementary to a human gene selected from CIITA, B2M and TRAC.

Disclosed herein, in some aspects are cell comprising an scAAV vector disclosed herein. In some embodiments, the cell is a T cell or a natural killer cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings described below.

FIG. 1 shows exemplary AAV vectors encoding small Cas effectors compared to an AAV vector encoding a Cas9 protein.

FIG. 2 shows gene editing tools that can fit into an AAV vector with small Cas effectors.

FIG. 3 shows the frequency of indel mutations generated in the PCSK9 gene in Hepal-6 cells with AAV vector encoding CasΦ.12 and a guide RNA.

FIG. 4 shows an exemplary AAV vector for restoring a wildtype sequence to the CFTR gene that has a cystic fibrosis causing mutation (CFTRdelF508).

FIG. 5A shows that a plasmid encoding a guide RNA and a Cas effector having a length of between 400 and 500 amino acids edits the genome of mammalian cells. FIG. 5B shows that this plasmid can edit the genome of mammalian cells at multiple doses.

FIG. 6A shows in vivo frequency of indel mutations generated in the PCSK9 gene in mice liver with AAV8 vector encoding CasΦ.12 and a guide RNA. FIG. 6B shows serum PCSK9 concentration in mouse post AAV8 vector injection. FIG. 6C shows effect of viral doses on generating indel mutations in the PCSK9 gene using the AAV vector encoding CasΦ.12 and the guide RNA.

FIG. 7 shows an exemplary schematic of AAV construct for gene editing according to one or more embodiments of the present disclosure. Included in FIG. 7 are the following abbreviations representing elements of the AAV construct: ITR=Inverted terminal repeat; gRNA=guide RNA; P1=first promoter; P2=second promoter; UTR=untranslated region; ssAAV=single-stranded AAV; scAAV=self-complementary AAV; and WPRE=Woodchuck Hepatitis Virus (WHV) posttranscriptional regulatory element.

FIGS. 8A-8C shows exemplary schematics of ssAAV and scAAV constructs for gene editing according to one or more embodiments of the present disclosure. FIG. 8A and FIG. 8B are ssAAV constructs, whereas FIG. 8C is an scAAV construct. Included in FIGS. 8A-8C are the following abbreviations representing elements of the AAV construct: gRNA=guide RNA; P1=first promoter; P2=second promoter; Cas=effector protein.

FIG. 9 shows the frequency of indel mutations generated in primary T cells with AAV vector encoding Cas19952 and a guide RNA at a ranging from 5e+02 to 5e+05.

FIG. 10A-10B show the conserved motifs that are shared by effector proteins. FIG. 10A shows weblogos of the multilevel consensus sequences of the conserved motifs. Weblogos corresponding to MEME_1, MEME_2, MEME_3, MEME_4, MEME_5, MEME_6 and MEME_7 are shown to the right of the “MEME” descriptor. FIG. 10B shows the location of the detected motifs in the effector proteins.

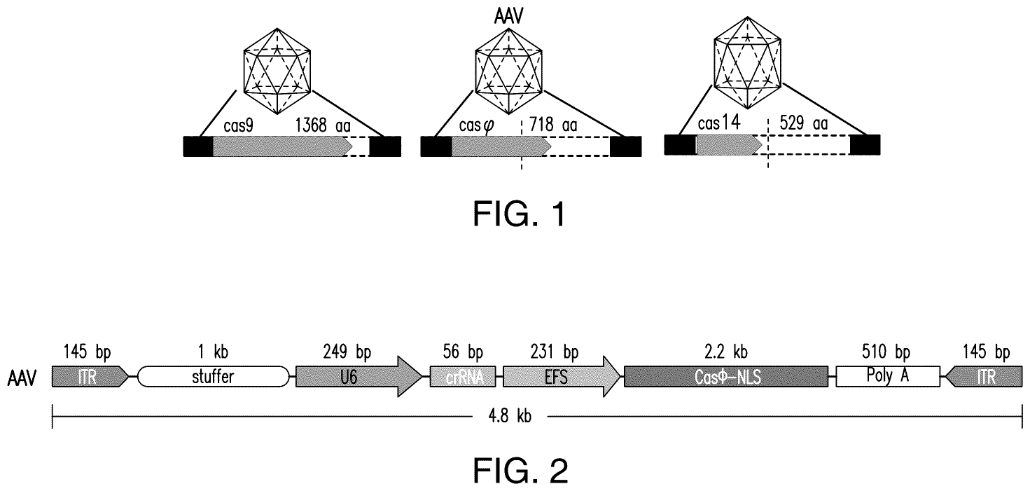

### DETAILED DESCRIPTION OF THE INVENTION

It is to be understood that both the foregoing general description and the following detailed description are exemplary and explanatory only and are not restrictive of the disclosure. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### I. Definitions

Unless otherwise indicated, all technical terms used herein have the same meaning as commonly understood by one of ordinary skill in the art to which this invention belongs. Unless otherwise indicated or obvious from context, the following terms have the following meanings:

As used in the specification and claims, the singular forms “a”, “an” and “the” include plural references unless the context clearly dictates otherwise. For example, the term “a sample” includes a plurality of samples, including mixtures thereof.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

“Percent identity,” “% identity,” and % “identical” refers to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 Mar;4(1):11-7), FASTA (Pearson and Lipman, Proc Nat Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The term “donor nucleic acid” refers to a polynucleotide in a vector that will be delivered into a cell. In some instances, the donor nucleic acid is integrated into the genome of the cell. The donor nucleic acid may be expressed (e.g., transcribed and/or translated) by the cell.

As used herein, the term, “donor subject,” refers to a subject that provides cells to a subject in need thereof.

The term “effector protein” refers to a protein that is capable of modifying a nucleic acid molecule (e.g., by cleavage, deamination, recombination). Modifying the nucleic acid may modulate the expression of the nucleic acid molecule (e.g., increasing or decreasing the expression of a nucleic acid molecule). The effector protein may be a Cas protein (i.e., an effector protein of a CRISPR-Cas system).

As used herein, the term “transgene” refers to the nucleic acid region of an AAV vector, including an scAAV vector, that falls between the two inverted terminal repeats (ITRs). The 5′ end of the transgene is directly adjacent to the 3′ end of the 5′ ITR, and the 3′ end of the transgene is directly adjacent to the 5′ end of the 3′ ITR. The transgene may comprise or consist essentially of genome editing components, including, e.g., a sequence encoding an effector protein, a sequence encoding a guide nucleic acid, and a donor nucleic acid.

The term “length” as it applies to a nucleic acid (polynucleotide) or polypeptide may be expressed as “kilobases” (kb) or “base pairs (bp),” and may be used interchangeably with the term, “linked nucleosides.” Thus, a length of 1 kb refers to a length of 1000 linked nucleosides, and a length of 500 bp refers to a length of 500 linked nucleosides. Similarly, a protein having a length of 500 linked amino acids may also be simply described as having a length of 500 amino acids.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some instances, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes place in a container for holding laboratory reagents such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

Disclosed herein are non-naturally occurring compositions and systems comprising an effector protein (e.g., an effector protein) and an engineered guide nucleic acid, which may simply be referred to herein as a guide nucleic acid. In general, the guide nucleic acid is at least one nucleic acid comprising: a first nucleotide sequence for interaction with an effector protein; and a second nucleotide sequence that hybridizes to a target nucleic acid. The first sequence may be non-covalently bound by an effector protein or hybridize to an additional nucleic acid, wherein the additional nucleic acid is non-covalently bound by the effector protein. The first sequence may be referred to herein as a repeat sequence or handle sequence. The second sequence may be referred to herein as a spacer sequence. A guide nucleic acid may be referred to interchangeably as a guide RNA, however it is understood that guide nucleic acids may comprise DNA, RNA, or a combination thereof (e.g., RNA with a thymine base).

As used herein, a nucleic acid can be described as a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more sequence modifications, or both. In some embodiments, the sequence modification of one or more nucleic acid residues of a nucleotide sequence or one or more amino acid residue of an amino acid sequence comprises chemical modification of one or more nucleobases; or chemical modifications to the phosphate backbone, a nucleotide, a nucleobase, or a nucleoside. Such modifications can be made to an effector protein amino acid sequence or guide nucleic acid nucleotide sequence or any sequence disclosed herein (e.g., a nucleic acid encoding an effector protein or a nucleic acid that, when transcribed, produces a guide nucleic acid). Methods of modifying a nucleic acid or amino acid sequence are known. One of ordinary skill in the art will appreciate that the sequence modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

In some embodiments, a target nucleic acid is a nucleic acid that is selected for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA). In some embodiments, the target nucleic acid comprises a target sequence. In some embodiments, the target sequence can hybridize to a respective length portion of a guide nucleic acid. Hybridization of the guide nucleic acid to the target sequence may bring an effector protein into contact with the target nucleic acid.

In general, an engineered effector protein and an engineered guide nucleic acid refer to an effector protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, non-naturally occurring and/or engineered are used interchangeably and indicate the involvement of the hand of man. In some cases, non-naturally occurring and/or an engineered composition (e.g., nucleic acid, nucleotide, protein, polypeptide, peptide or amino acid, refer to a nucleic acid, nucleotide, protein, polypeptide, peptide or amino acid) is at least substantially free from at least one other feature with which it is naturally associated in nature and as found in nature, and/or contains a modification (e.g., chemical modification, nucleotide sequence, or amino acid sequence) that is not present in the naturally occurring composition (e.g., nucleic acid, nucleotide, protein, polypeptide, peptide, or amino acid). In some embodiments, the terms, when referring to a composition or system described herein, is a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is natural, naturally-occurring, and/or found in nature includes an effector protein and a guide nucleic acid from a cell or organism that have not been genetically modified by the hand of man.

In some embodiments, the guide nucleic acid comprises two heterologous sequences arranged in an order or proximity that is not observed in nature. Therefore, compositions and systems described herein are not naturally occurring. In some instances, the guide nucleic acid comprises a non-natural nucleobase sequence. In some instances, the non-natural sequence is a nucleobase sequence that is not found in nature. The non-natural sequence may comprise a portion of a naturally occurring sequence, wherein the portion of the naturally-occurring sequence is not present in nature, absent the remainder of the naturally-occurring sequence. In some instances, the guide nucleic acid comprises two naturally occurring sequences arranged in an order or proximity that is not observed in nature. In some instances, compositions and systems comprise a ribonucleotide complex comprising an effector protein and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally-occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence located at a 3′ or 5′ end of the guide nucleic acid, or between the first and second sequences of the guide nucleic acid. For example, an engineered guide nucleic acid may comprise a naturally occurring CRISPR RNA (crRNA), wherein the guide nucleic acid comprises a linker sequence. In some embodiments, crRNA and trans-activating crRNA (tracrRNA) are coupled by a linker sequence. In some embodiments, tracrRNA comprises a first sequence that is capable of being non-covalently bound by an effector protein. TracrRNAs may comprise a second sequence that hybridizes to a portion of a crRNA, which may be referred to as a repeat hybridization sequence.

In some embodiments, a guide nucleic acid comprises a single guide nucleic acid, also referred to as a single guide RNA (sgRNA). In some embodiments, sgRNA comprises a handle sequence. In some embodiments, a handle sequence binds non-covalently to an effector protein. In some instances, the handle sequence comprises all or a portion of a repeat sequence. In some embodiments, a handle sequence includes a portion of a tracrRNA sequence that is capable of being non-covalently bound by an effector protein. In some embodiments, a handle sequence does not include all or a part of the portion of a tracrRNA that hybridizes to a portion of a crRNA as found in a dual nucleic acid system. In some embodiments, a handle sequence can include a portion of a tracrRNA sequence as well as a portion of a repeat sequence, which can optionally be connected by a linker. In some embodiments, a handle sequence in the context of a sgRNA can also be described as the portion of the sgRNA that does not hybridize to a target sequence in a target nucleic acid (e.g., a spacer sequence). As used herein, the term “linker” or “linker sequence” refers to a covalent bond or molecule that links a first polypeptide to a second polypeptide or a first nucleic acid to a second nucleic acid.

A person of ordinary skill in the art would appreciate that referring to a “nucleotide(s)”, and/or “nucleoside(s)”, in the context of a nucleic acid molecule having multiple residues, is interchangeable and describe the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all ofthymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are complementary to the same sequence (5′-CAU).

In some instances, compositions and systems described herein comprise an engineered effector protein that is similar to a naturally occurring effector protein. The engineered effector protein may lack a portion of the naturally occurring effector protein. The effector protein may comprise a mutation relative to the naturally occurring effector protein, wherein the mutation is not found in nature. The effector protein may also comprise at least one additional amino acid relative to the naturally occurring effector protein. For example, the effector protein may comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

### II. Introduction

Disclosed herein are compositions, systems and methods comprising a viral vector, wherein the viral vector comprises: a nucleic acid encoding the polypeptide (e.g., effector protein); and a guide nucleic acid or a nucleic acid encoding the guide nucleic acid.

Further described herein are viral vectors encoding polypeptides (e.g., effector protein) that can recognize and, optionally, cleave nucleic acids in a sequence-specific manner when complexed with a guide nucleic acid. Such a polypeptide (e.g., effector protein) and guide nucleic acid complex can hybridize a target region of a target nucleic acid and cleave the target nucleic acid at a position adjacent to the target region. In some embodiments, polypeptide (e.g., effector protein) can be activated when it binds a target region of a target nucleic acid to cleave regions of the nucleic acid that are near, but not adjacent to the target region. A polypeptide can be an effector protein, such as a CRISPR-associated (Cas) protein, which may be coupled to a guide nucleic acid that imparts activity or sequence selectivity to the polypeptide.

A person skilled in the art would understand that the terms cleave, cleaving, and cleavage are interchangeable. In some embodiments, when used in reference to a nucleic acid molecule or nuclease activity of an effector protein, cleavage can be a hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

A person skilled in the art would understand that the terms hybridize and hydbrizable are interchangeable. In some embodiments, a hybridized sequence of nucleotides can be a nucleotide sequence that is able to noncovalently bind, i.e. form Watson-Crick base pairs and/or G/U base pairs, or anneal, to another nucleotide sequence in a sequence-specific, antiparallel, manner (i.e., a nucleotide sequence specifically binds to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, or for hybridization to occur.

Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and TABLE 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

A person skilled in the art would understand that the terms complementary and complementarity are interchangeable. In some embodiments, when used in reference to a nucleic acid molecule or nucleotide sequence, complementarity can be a characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the reverse complement sequence or the reverse complementary sequence is understood as the sequence of the lower strand in the direction of its 5′- to its 3′-end. Each nucleotide in a double stranded DNA or RNA molecule that is paired with its Watson-Crick counterpart called its complementary nucleotide.

A person skilled in the art would understand that the terms bind and binding are interchangeable.

In some embodiments, binding can be a non-covalent interaction between macromolecules (e.g., between two polypeptides, between a polypeptide and a nucleic acid; between an effector protein/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

In general, guide nucleic acids comprise a CRISPR RNA (crRNA) that is at least partially complementary to a target region in a target nucleic acid. A person skilled in the art would appreciate that crRNA can be a type of guide nucleic acid, wherein the nucleic acid is a RNA. Such a crRNA, in some embodiments, can comprise a first sequence, often referred to herein as a spacer sequence, that hybridizes to a target sequence of a target nucleic acid, and a second sequence, often referred to herein as a repeat sequence, that interacts with an effector protein. In some embodiments, the second sequence is bound by the effector protein. In some embodiments, compositions, systems, and methods comprising effector proteins and guide nucleic acids can further comprise a trans-activating crRNA (tracrRNA), at least a portion of which interacts with the effector protein. In some embodiments, the second sequence hybridizes to a portion of a tracrRNA. In some embodiments, the tracrRNA is provided separately from the guide nucleic acid. In some embodiments, the tracrRNA forms a complex with the effector protein. In general, the guide nucleic acid (e.g., crRNA) does not comprise a tracrRNA.

Polypeptides (e.g., effector proteins) disclosed herein may cleave nucleic acids, including single stranded RNA (ssRNA), double stranded DNA (dsDNA), and single-stranded DNA (ssDNA). Polypeptides (e.g., effector proteins) disclosed herein may provide cis cleavage activity, trans cleavage activity, nickase activity, or a combination thereof. Cis cleavage activity is cleavage of a region of a target nucleic acid that is hybridized to at least a portion of the guide RNA (crRNA or sgRNA), wherein cleavage occurs within or directly adjacent to the region of the target nucleic acid that is hybridized to at least the portion of the guide RNA. Trans cleavage activity (also referred to as transcollateral cleavage) is cleavage of ssDNA or ssRNA that is near, but not hybridized to the guide RNA. Alternatively, trans cleavage refers to a cleavage (hydrolysis of a phosphodiester bond) of one or more non-target nucleic acids by an effector protein that is complexed with a guide nucleic acid and a target nucleic acid. Trans cleavage activity is triggered by the hybridization of guide RNA to the target nucleic acid. Nickase activity is the selective cleavage of one strand of a dsDNA molecule.

Effector proteins as described herein, through their ability to cleave DNA at a precise target location in the genome of a wide variety of cells and organisms, allow for precise and efficient editing of DNA sequences of interest. Single strand breaks and double strand breaks can be an effective way to disrupt a gene of interest, generate DNA or RNA modifications, and to treat genetic disease through gene correction.

Disclosed herein are non-naturally occurring compositions, systems and methods comprising a viral vector encoding at least one of an engineered polypeptide or effector protein and an engineered guide nucleic acid, which may simply be referred to herein as a polypeptide or effector protein and a guide nucleic acid, respectively. In general, an effector protein and a guide nucleic acid refer to an effector protein and a guide nucleic acid, respectively, that are not found in nature. In some embodiments, compositions, methods and systems described herein comprise a viral vector encoding at least one non-naturally occurring component. For example, disclosed compositions, methods and systems may comprise a viral vector encoding a guide nucleic acid, wherein the sequence ofthe guide nucleic acid is different or modified from that of a naturally-occurring guide nucleic acid. In some embodiments, disclosed compositions, systems and methods comprise a viral vector encoding at least two components that do not naturally occur together. For example, disclosed compositions, methods and systems may comprise a viral vector encoding a guide nucleic acid comprising a repeat region and a spacer region which do not naturally occur together. Also, by way of example, disclosed compositions, methods and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural sequence is a nucleotide sequence that is not found in nature. The non-natural sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. Therefore, compositions described herein are not naturally occurring.

In some embodiments, a guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid. A protein that is heterologous to the effector protein is a protein that is not covalently linked via an amide bond to the effector protein in nature. In some embodiments, a heterologous protein is not encoded by a species that encodes the effector protein.

In some embodiments, compositions and systems described herein comprise a viral vector encoding an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. For example, the effector protein may comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

### III. Viral Vectors

Disclosed herein, in some aspects, are compositions, systems, and methods comprising a viral vector, wherein the viral vector encodes an effector protein and a guide nucleic acid. In some embodiments, a viral vector is a nucleic acid to be delivered into a host cell via a recombinantly produced virus or viral particle. Alternatively, in some embodiments, recombinant technology is used to produce virus or viral particle, wherein the recombinant technology uses various combinations of cloning, restriction, and/or ligation steps resulting in a construct having a nucleic acid distinguishable from endogenous nucleic acids found in natural systems. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

There are a variety of viral vectors that are associated with various types of viruses, including but not limited to lentiviruses, adenoviruses and adeno-associated viruses. Often the viral vectors of the instant disclosure are an adeno-associated viral vector (AAV vector). Generally, an AAV vector has two inverted terminal repeats (ITRs). According, in some embodiments, the viral vector provided herein comprises two inverted terminal repeats of AAV. Typically, the length of each ITR is about 145 bp. The DNA sequence in between the ITRs may be referred to as the “transgene” or the sequence encoding the “genome editing tools.” The genome editing tools may include, but are not limited to, an effector protein, effector protein modifications (e.g., nuclear localization signal (NLS), polyA tail), a guide nucleic acid, respective promoter(s), and a donor nucleic acid, or combinations thereof. In some instances, the length of the transgene (also referred to as the cloning capacity) between the ITRs is about 4 kb to about 5 kb. In some instances, the length of the transgene is about 4.2 kb to about 4.8 kb. In some instances, the length of the transgene is about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, about 3.3 kb, about 3.4 kb, about 3.5 kb, about 3.6 kb, about 3.7 kb, about 3.8 kb, about 3.9 kb, about 4.0 kb, about 4.1 kb, about 4.2 kb, about 4.3 kb, about 4.4 kb, about 4.5 kb, about 4.6 kb, about 4.7 kb, about 4.8 kb, about 4.9 kb, or about 5 kb.

In some embodiments, the transgene is inserted into a cell for expression of said nucleotide sequence in the cell. A transgene may comprise (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced, or a combination thereof. In some embodiments, the transgene comprises a nucleotide sequence encoding a wildtype protein. In some embodiments, a transgene comprises a donor nucleic acid. The cell in which transgene expression occurs may be referred to as a target cell or a host cell.

In some instances, the AAV vector is a self-complementary AAV (scAAV) vector. The coding region of the scAAV vector forms an intramolecular double-stranded DNA template. In general, the transgene of an scAAV vector has a length of about 2 kb to about 3 kb. In some instances, the length of the transgene of an scAAV vector is about 2 kb, about 2.1 kb, about 2.2 kb, about 2.3 kb, about 2.4 kb, about 2.5 kb, about 2.6 kb, about 2.7 kb, or about 2.8 kb. The scAAV vector may comprise a nucleic acid encoding the effector protein and a guide nucleic acid. In some instances, the guide nucleic acid comprises or consists essentially of a crRNA. In some instances, the scAAV vector encodes a crRNA and a tracrRNA. In some instances, the guide nucleic acid is single guide RNA. In some instances, the scAAV does not encode a tracrRNA.

In some instances, the AAV vector is a self-inactivating AAV vector. In some instances, the self-inactivating AAV vector is a self-inactivating scAAV vector. In some instances, the self-inactivating AAV vector comprises a guide nucleic acid, wherein the guide nucleic acid comprises a region that is complementary to the region of the AAV vector encoding the effector protein. In some instances, the AAV vector comprises one or more guide nucleic acids that comprise a region that is complementary to sequences near the 5′ and 3′ ends of the region of the AAV vector encoding the effector protein, thereby allowing for the region of the AAV vector encoding the effector protein to be excised. Thus, the effector protein may control expression of itself, in some instances limiting the duration of expression of the effector protein, thereby limiting off-target effector protein activity and enabling safe genome editing.

In some cases, an AAV vector comprises a modification such as an insertion, deletion, chemical alteration, or synthetic modification. In some cases, the modification is in a protein coding region or a non-coding region of an AAV vector (e.g., relative to a wild-type vector). In some cases, a modification improves the protein expression activity ofthe AAV vector. In some cases, an AAV vector provided herein is chimeric. In some cases, inverted terminal repeats of an AAV vector comprise a 5′ inverted terminal repeat, a 3′ inverted terminal repeat, and a mutated inverted terminal repeat. In some cases, a mutated inverted terminal repeat lacks a terminal resolution site. In some cases, an AAV vector provided herein comprises a modification in a capsid or rep protein. In some cases, an AAV vector provided herein comprises a modification which reduces immunogenicity (e.g., relative to a wild-type vector) to allow for repeated dosing. In some cases, methods provided herein comprise administering to a subject a composition comprising a vector of a first serotype in a first dose and a vector of a second serotype in a second to dose to reduce and/or eliminate immunogenicity (e.g., compared to a method of administering vectors of the same serotype in first and second doses). In some instances, an AAV vector provided herein comprises any combination of rep, cap, and ITR sequences from different AAV serotypes. In some cases, an AAV vector comprises a genome comprising a replication gene and inverted terminal repeats from a first AAV serotype and a capsid protein from a second AAV serotype.

### AAV Viruses

In general, the AAV vector is delivered by an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof. In some instances, the AAV serotype is AAV-DJ. AAV-DJ is a synthetic serotype with a chimeric capsid of AAV-2, 8 and 9, see Grimm et al. J Virol. 2008 Jun;82(12):5887-911 for additional description of AAV-DJ. In some instances, the AAV serotype is a AAV-retro serotype. An AAV-retro serotype virus infects neuronal cell bodies, is internalized by axons and mediates retrograde access to projection neurons. Non-limiting examples of AAV-retro are described by Tervo et al (2016) Neuron, vol. 92, pp. 372-382, and Lin et al., (2020), Molecular Brain vol. 13, No. 138.

In some cases, an AAV provided herein is engineered or modified. In some cases, a modification comprises a deletion, insertion, mutation, substitution, or a combination thereof of the capsid protein, the rep protein, an ITR sequence, or other components of an AAV. In some cases, modifications to the AAV genome and/or the capsids/rep proteins can be designed to facilitate more efficient or more specific transduction of cells or tissues for gene therapy. In general, an AAV undergoes several steps prior to achieving gene expression: 1) binding or attachment to cellular surface receptors, 2) endocytosis, 3) trafficking to the nucleus, 4) uncoating of the virus to release the genome, and 5) conversion of the genome from single-stranded to double-stranded DNA as a template for transcription in the nucleus. In some instances, the cumulative efficiency with which an AAV can successfully execute each individual step can determine the overall transduction efficiency. In some cases, modifications of AAV may improve or modify the rate limiting steps in AAV transduction including the absence or low abundance of required cellular surface receptors for viral attachment and internalization, inefficient endosomal escape leading to lysosomal degradation, slow conversion of single-stranded to double-stranded DNA template, or a combination thereof.

In some cases, an AAV viral capsid is modified relative to a naturally occurring AAV viral capsid.

In some cases, modifying an AAV viral capsid comprises modifying a combination of capsid components. In some cases, a mutated AAV virus particle comprises a mutation in at least one capsid protein. In some cases, the mutation is in VP1 and VP2, in VP1 and VP3, in VP2 and VP3, or in VP1, VP2, and VP3. In some cases, a VP is eliminated. A mutation can occur at any of AAV capsid positions described thereof and can include any number of mutations. In some cases, a mutation is from one amino acid to another amino acid. A mutation may comprise modifying an amino acid to any permutation of the canonical amino acids (e.g., relative to a wildtype capsid protein). Any of the following amino acid modifications can be made at any of VP1, VP2, and VP3: A to R, A to N, A to D, A to C, A to Q, A to E, A to G, A to H, A to I, A to L, A to K, A to M, A to F, A to P, A to S, A to T, A to W, A to Y, A to V, R to N, R to D, R to C, R to Q, R to E, R to G, R to H, R to I, R to L, R to K, R to M, R to F, R to P, R to S, R to T, R to W, R to Y, R to V, N to D, N to C, N to Q, N to E, N to G, N to H, N to I, N to L, N to K, N to M, N to F, N to P, N to S, N to T, N to W, N to Y, N to V, D to C, D to Q, D to E, D to G, D to H, D to I, D to L, D to K, D to M, D to F, D to P, D to S, D to T, D to W, D to Y, D to V, C to Q, C to E, C to G, C to H, C to I, C to L, C to K, C to M, C to F, C to P, C to S, C to T, C to W, C to Y, C to V, Q to E, Q to G, Q to H, Q to I, Q to L, Q to K, Q to M, Q to F, Q to P, Q to S, Q to T, Q to W, Q to Y, Q to V, E to G, E to H, E to I, E to L, E to K, E to M, E to F, E to P, E to S, E to T, E to W, E to Y, E to V, G to H, G to I, G to L, G to K, G to M, G to F, G to P, G to S, G to T, G to W, G to Y, G to V, H to I, H to L, H to K, H to M, H to F, H to P, H to S, H to T, H to W, H to Y, H to V, I to L, I to K, I to M, I to F, I to P, I to S, I to T, I to W, I to Y, I to V, L to K, L to M, L to F, L to P, L to S, L to T, L to W, L to Y, L to V, K to M, K to F, K to P, K to S, K to T, K to W, K to Y, K to V, M to F, M to P, M to S, M to T, M to W, M to Y, M to V, F to P, F to S, F to T, F to W, F to Y, F to V, P to S, P to T, P to W, P to Y, P to V, S to T, S to W, S to Y, S to V, T to W, T to Y, T to V, W to Y, W to V, Y to V, and any of the previously described mutations in reverse.

In some cases, a vector provided herein comprises a chimeric capsid. In some cases, a chimeric capsid comprises an insertion of a foreign protein sequence into the open reading frame of the capsid gene, either from another wild-type (wt) AAV sequence or an unrelated protein. In some cases, a chimeric capsid is produced using a naturally existing serotype as a template. In some instances, a chimeric capsid is produced using a serotype that is mutated relative to a wild type as a template. In some cases, a chimeric capsid can comprise at least one capsid polypeptide from an AAV serotype comprising AAV1, AAV2, AAV3, AAV4, AAV5, AAV6, AAV7, AAV8, AAV9, AAV10, AAV11, or AAV12. In some cases, a viral vector provided herein comprises a polypeptide comprising a VP1 from an AAV serotype comprising AAV1, AAV2, AAV3, AAV4, AAV5, AAV6, AAV7, AAV8, AAV9, AAV10, AAV11, or AAV12. In other cases, a viral vector provided herein comprises a polypeptide comprising a VP2 from an AAV comprising AAV1, AAV2, AAV3, AAV4, AAV5, AAV6, AAV7, AAV8, AAV9, AAV10, AAV11, or AAV12. In some cases, a viral vector provided herein comprises a polypeptide comprising a VP3 from an AAV serotype comprising AAV1, AAV2, AAV3, AAV4, AAV5, AAV6, AAV7, AAV8, AAV9, AAV10, AAV11, or AAV12.

In some instances, the AAV targets a cell. In some instances, the AAV is capable of infecting a cell. In some instances, the cell is a blood cell. The blood cell may be a leukocyte. The leukocyte may be a T cell. The cell may be a hematopoietic stem cell (HSC). In some instances, the cell is a hepatocyte. In some instances, the cell is a lung cell. In some instances, the cell is a neuron. In some instances, the cell is a stem cell. In some instances, the cell is a pluripotent cell. In some instances, the cell is an epithelial cell. In some instances, the cell is a part of a target tissue or produced by a target tissue. In some instances, the target tissue of AAV1, AAV2, AAV4, AAV5, AAV8, or AAV9 viruses comprises retinal pigment epithelium. In some cases, the target tissue of an AAV1, AAV2, AAV4, AAV5, AAV8, or AAV9 vector comprises the central nervous system. In some cases, the target tissue of an AAV1, AAV6, AAV7, AAV8, or AAV9 vector comprises skeletal muscle. In some cases, the target tissue of an AAV1, AAV8, or AAV9 vector comprises the heart. In some cases, the target tissue of an AAV2 vector comprises kidney, joint or brain tissue. In some cases, the target tissue of an AAV2, AAV5, or AAV8 vector comprises photoreceptor cells. In some cases, the target tissue of an AAV4, AAV5, AAV6, AAV9 vector comprises the lung. In some cases, the target tissue of an AAV2, AAV5, AAV7, AAV8, or AAV9 vector comprises the liver. In some cases, the target tissue of an AAV8 vector comprises a pancreas. Details of selecting an AAV vector based on the target tissues are provided in, for example, Srivastava, Curr. Opin. Virol., 2016 Dec;21:75-80 and Benskey et al., 2019, Viral Vectors for Gene Therapy, Humana Press, each of which is incorporated by reference in their entireties.

### Producing AAV

The AAV vectors described herein may be referred to as recombinant AAV (rAAV). Often, rAAV particles are generated by transfecting AAV producing cells with an AAV-containing plasmid carrying the transgene, a plasmid that carries viral encoding regions, i.e., Rep and Cap gene regions; and a plasmid that provides the helper genes such as E1A, E1B, E2A, E4ORF6 and VA. In some instances, the AAV producing cells are mammalian cells. In some instances, host cells for rAAV viral particle production are mammalian cells. In some cases, a mammalian cell for rAAV viral particle production is a COS cell, a HEK293T cell, a HeLa cell, a KB cell, a derivative thereof, or a combination thereof. In some cases, rAAV virus particles may be produced in the mammalian cell culture system by providing the rAAV plasmid to the mammalian cell. In some cases, producing rAAV virus particles in a mammalian cell may comprise transfecting vectors that express the rep protein, the capsid protein, and the gene-of-interest expression construct flanked by the ITR sequence on the 5′ and 3′ ends. Methods of such processes are provided in, for example Naso et al., BioDrugs, 2017 Aug;31(4):317-334 and Benskey et al., 2019, Viral Vectors for Gene Therapy, Humana Press, each of which is incorporated by reference in their entireties.

In some instances, rAAV is produced in a non-mammalian cell. In some instances, rAAV is produced in an insect cell. In some cases, an insect cell for producing rAAV viral particles comprises a Sf9 cell. In some cases, production of rAAV virus particles in insect cells may comprise baculovirus. In some cases, production of rAAV virus particles in insect cells may comprise infecting the insect cells with three recombinant baculoviruses, one carrying the cap gene, one carrying the rep gene, and one carrying the gene-of-interest expression construct enclosed by an ITR on both the 5′ and 3′ end. In some cases, rAAV virus particles are produced by the One Bac system. In some cases, rAAV virus particles may be produced by the Two Bac system. In some instances, in the Two Bac system, the rep gene and the cap gene of the AAV is integrated into one baculovirus virus genome, and the ITR sequence and the gene-of-interest expression construct is integrated into another baculovirus virus genome. In some cases, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., Mol. Cell. Biol., 1983 Dec;3(12):2156-65; Urabe et al. Hum. Gene. Ther., 2002, Nov;1;13(16):1935-43; and Benskey et al., 2019, Viral Vectors for Gene Therapy, Humana Press, each of which is incorporated by reference in its entirety.

In some embodiments, viral vectors comprise one or more regulatory elements. In some embodiments, the regulatory element comprises one or more transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

In general, viral vectors comprise at least one promoter. As used herein, the term “promoter” or “promoter sequence” is a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

Promoters can be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 10 fold, by 100 fold, or by 1000 fold, or more. In addition, vectors used for providing a nucleic acid that, when transcribed, produces an guide nucleic acid and/or a nucleic acid that encodes an effector protein to a cell may include nucleic acid sequences that encode for selectable markers in the target cells, so as to identify cells that have taken up the guide nucleic acid and/or an effector protein.

In some instances, the viral vector comprises a first promoter that drives expression of the effector protein. In some instances, the viral vector comprises a second promoter that drives expression of the guide nucleic acid. In some instances, the length of the promoter is less than about 500, less than about 400, or less than about 300 linked nucleosides. In some instances, the length of the promoter is at least 100 linked nucleosides. Non-limiting examples of promoters include ApoE, TBG, CMV, 7SK, EF1a, RPBSA, hPGK, EFS, SV40, PGK1, hPGK, Ube, human beta actin promoter, CAG, TRE, UAS, Ac5, Polyhedrin, CaMKIIa, GAL1-10, H1, TEF1, GDS, ADH1, CaMV35S, Ubi, U6, MNDU3, Ck8e, SPC5-12, Desmin, MND, and MSCV. In some instances, the promoter for the guide nucleic acid is a U6 promoter, having a length of about 249 linked nucleosides. In some instances, the promoter for the effector protein is an EFS promoter, having a length of about 231 linked nucleosides. In some instances, the promoter is a tissue-specific promoter that has activity in only certain cell types. In some instances, the cell type is a hepatocyte, an epithelial cell, an endothelial cell, a leukocyte, a lymphocyte, a pancreatic cell, a kidney cell, a neuron, a pluripotent cell, a stem cell, a muscle cell, a heart cell, a skin cell, or a lung cell. In some instances, the cell is an immune cell e.g., a macrophage, a monocyte, a Kupffer cell, a microglial cell, a neutrophil, an eosinophil, a basophil, a mast cell, a dendritic cell, a natural killer cell, a B cell or a T cell. Non-limiting examples of tissue-specific promoters include those that drive expression of the following genes B29 (in B cells); BD14 (in monocytes); CD43 (in leukocytes and platelets); CD45, IFN-beta, WASP (in hematopoietic cells); CD68 (in macrophages); desmin, Mb (in muscle cells); elastase-1 (in pancreatic cells); endoglin, Flt-1, ICAM-2, Tie2 (in endothelial cells); fibronectin (in skin); GFAP (in astrocytes); NphsI (in podocytes); OG-2 (in osteoblasts); SP-B (in lung cells); SYN-1 (in neurons); and SV-40, bAlb (in liver). In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a liver-specific promoter.

In some embodiments, the liver-specific promoter comprises ApoE or TBG promoter sequence. In some embodiments, the promoter for expressing effector protein is a ubiquitous promoter. In some embodiments, the ubiquitous promoter comprises MND or CAG promoter sequence.

In some instances, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44 Donor Nucleic Acids.

In some instances, viral vectors comprise a synthetic promoter that has been designed to control expression of a coding sequence in the viral vector (e.g., the effector protein, guide nucleic acid and/or donor nucleic acid). In some instances, the sequence of the synthetic promoter is not a sequence that exists in nature. In some instances, the synthetic promoter comprises a cis-regulatory sequence derived from one or more naturally-occurring promoter elements. In some instances, synthetic promoters are short, e.g. less than 150 or less than 100 nucleotides in length. In some instances, synthetic promoters comprise a transcription binding site element. In some instances, synthetic promoters comprise two or more transcription binding site elements found in a genome, but that do not correspond to the same gene within that genome. A non-limiting example of a synthetic promoter comprises multiple copies of NFAT and AP1 promoter binding sites. This NFAT/AP1 promoter may be especially useful in immune cells such as T cells. Synthetic promoters are further described by Ali et al. (2019) Front. Plant Sci., vol. 10, article 1433.

### All-In-One CAR T+CRISPR KO AAV

Provided herein are viral vectors encoding an effector protein, a guide nucleic acid, and a donor nucleic acid, wherein the donor nucleic acid encodes a chimeric antigen receptor (CAR). Also provided herein are viruses comprising such viral vectors. Also provided herein are methods of contacting cells with such viral vectors and viruses. Also provided herein are cells that have been modified with such viral vectors and viruses, referred to herein as “CAR T-cells.” In general, CAR T-cells are T cells that express a CAR. A CAR T-cell may be activated in the presence of its respective antigen on a target cell, resulting in the destruction of the target cell. The term, CAR T-cells, includes natural killer (NK) cells that express a CAR. In some embodiments, the T cell expresses CD4 (also referred to as a “CD4+ T cell”). In some instances, the T cell expresses CD8 (also referred to as a “CD8+ T cell”). In some instances, the T cell expresses CD4 and CD8 (also referred to as a “CD4+CD8+ T cell”). A T cell includes all types of immune cells expressing CD3, including: naïve T cells (cells that have not encountered their cognate antigens), T-helper cells (CD4+ cells), cytotoxic T-cells (CD8+ cells), natural killer T-cells, T-regulatory cells (T-reg) and gamma-delta T cells. Non-limiting exemplary sources for commercially available T cell lines include the American Type Culture Collection, or ATCC, and the German Collection of Microorganisms and Cell Cultures.

In general, a CAR comprises an antigen binding domain that is expressed on the surface of the CAR T-cell. In some embodiments, a CAR comprises a fused protein comprising an extracellular domain capable of binding to an antigen, a transmembrane domain derived from a polypeptide different from a polypeptide from which the extracellular domain is derived, and at least one intracellular domain. In some embodiments, a CAR can be referred to as a chimeric receptor, a T-body or a chimeric immune receptor (CIR). In some cases, the extracellular domain capable of binding to an antigen is any oligopeptide or polypeptide (e.g., antibody binding domain(s)) that can bind to an antigen. In some cases, the transmembrane domain is any oligopeptide or polypeptide known to span the cell membrane and link the extracellular domain and the signaling domain. In some cases, the intracellular domain is any oligopeptide or polypeptide known to function as a domain that transmits a signal to cause activation or inhibition of a biological process in a cell (primary signaling domain). In some instances, the intracellular domain may include one or more costimulatory signaling domains in addition to the primary signaling domain. A CAR may also include a hinge domain that serves as a linker between the extracellular and transmembrane domains.

The antigen binding domain may be considered to be an extracellular domain. In general, the antigen binding domain binds an antigen on a target cell. The antigen binding domain may comprise an antibody. The antibody may comprise an immunoglobulin or antigen binding fragment thereof. The antibody may be a polyclonal antibody or a monoclonal antibody. The antigen binding domain may comprise or consist essentially of an antigen binding antibody fragment, referred to simply herein as an antibody fragment. Non-limiting examples of antibody fragments include Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), and isolated CDRs.

In some instances, the target cell is a damaged cell or a cell affected by a disease. In some instances, the target cell is an immune cell (T cell or B cell) and the disease is an autoimmune cell. In some instances, the target cell is an infected cell, and the CAR recognizes an antigen expressed on the surface ofthe infected cell due to the infection/pathogen (e.g., hepatitis virus, human immunodeficiency virus, influenza virus and corona virus). The target cell may be a circulating cell, including, but not limited to, a leukocyte, lymphocyte or metastatic cell. The target cell may be part of a solid tumor. The target cell may be a benign cell. The target cell may be a pluripotent cell. The target cell may be a cell of the blood, bone, heart, lung, liver, esophagus, intestine, colon, bladder, stomach, pancreas, kidney, adrenal gland, thyroid gland, prostate gland, brain, nervous system, skeletal muscle, endothelium, vascular smooth muscle, adipose tissue, ovary, uterus, breast, testes, or immune system.

In some instances, the target cell is a cancer cell. A cancer cell may be a cell harboring one or more mutations that results in unchecked proliferation of the cancer cell. Such mutations are known in the art. Non-limiting examples of antigens are ADRB3, AKAP-4, ALK, Androgen receptor, B7H3, BCMA, BORIS, BST2, CAIX, CD179a, CD123, CD171, CD19, CD20, CD22, CD24, CD30, CD300LF, CD33, CD38, CD44v6, CD72, CD79a, CD79b, CD97, CEA, CLDN6, CLEC12A, CLL-1, CS-1, CXORF61, CYP1B1, Cyclin B1, E7, EGFR, EGFRvIII, ELF2M, EMR2, EPCAM, ERBB2 (Her2/neu), ERG (TMPRSS2 ETS fusion gene), ETV6-AML, EphA2, Ephrin B2, FAP, FCAR, FCRL5, FLT3, Folate receptor alpha, Folate receptor beta, Fos-related antigen 1, Fucosyl GM1, GD2, GD3, GM3, GPC3, GPR20, GPRC5D, GloboH, HAVCR1, HMWMAA, HPV E6, IGF-I receptor, IL-13Ra2, IL-11Ra, KIT, LAGE-1a, LAIR1, LCK, LILRA2, LMP2, LY6K, LY75, LewisY, MAD-CT-1, MAD-CT-2, MAGE A1, MAGE-A1, ML-IAP, MUC1, MYCN, MelanA/MARTI, Mesothelin, NA17, NCAM, NY-BR-1, NY-ESO-1, OR51E2, OY-TES 1, PANX3, PAP, PAX3, PAX5, PCTA-1/Galectin 8, PDGFR-beta, PLAC1, PRSS21, PSCA, PSMA, Polysialic acid, Prostase, RAGE-1, ROR1, RU1, RU2, Ras mutant, RhoC, SART3, SSEA-4, SSX2, TAG72, TARP, TEM1/CD248, TEM7R, TGS5, TRP-2, TSHR, Tie 2, Tn Ag, UPK2, VEGFR2, WT1, XAGE1, and IGLL1.

In general, a CAR comprises an intracellular binding domain. The intracellular binding domain generally contributes to the activation of the CAR T-cell when the antigen binding domain of the CAR associates with its respective antigen. In some embodiments, the intracellular signaling domain of said CAR comprises a functional signaling domain of a protein selected from the group consisting of 4-1BB (CD137), B7-H3, BAFFR, BLAME (SLAMF8), CD100 (SEMA4D), CD103, CD150, CD160, CD160 (BY55), CD162 (SELPLG), CD18, CD19, CD2, CD229, CD27, CD28, CD29, CD30, CD4, CD40, CD49D, CD49a, CD49f, CD69, CD7, CD84, CD8alpha, CD8beta, CD96, CDS, CD11a, CD11b, CD11c, CD11d, CEACAM1, CRTAM, DNAM1 (CD226), GADS, GITR, HVEM (LIGHTR), IA4, ICAM-1, ICOS, IL2R beta, IL2R gamma, IL7R alpha, ITGA4, ITGA4, ITGA6, ITGAD, ITGAE, ITGAL, ITGAM, ITGAX, ITGB1, ITGB2, ITGB7, LAT, LFA-1, LFA-1, LIGHT, LTBR, NKG2C, NKp30, NKp44, NKp46, NKp80 (KLRF1), OX40, PAG/Cbp, PD-1, PSGL1, SLAMF1, SLAMF4, SLAMF6, SLAMF7, SLP-76, TNFR2, TRANCE/RANKL, VLA1, and VLA-6.

In some instances, the length of the donor nucleic acid encoding the CAR is about 0.5 kb to about 1 kb, about 1 kb to about 1.5 kb, about 1.5 kb to about 2 kb, or about 2 kb to about 2.5 kb. In some instances, the length of the donor nucleic acid is about 1 kb to about 2 kb. In some instances, the length of the donor nucleic acid is about 2.5 kb to about 5 kb. In some instances, the length of the donor nucleic acid is about 1 kb to about 1.2 kb, about 1.2 kb to about 1.6 kb, about 1 kb to about 1.2 kb, about 1.2 kb to about 1.4 kb, about 1.4 kb to about 1.6 kb, about 1.6 kb to about 1.8 kb, about 1.8 kb to about 2 kb.

In some instances, the guide nucleic acid comprises a sequence that is at least partially identical or complementary to an equal length portion of at least one human leukocyte antigen (HLA) gene. In some instances, the HLA is expressed by a subject who is serving as a T cell donor to a subject in need of T cells.

Such modified T cells may be referred to as allogeneic T cells. The HLA may be an HLA corresponding to MHC class I, e.g., HLA-A, HLA-B, or HLA-C). The HLA may be an HLA corresponding to MHC class II, e.g., HLA-DP, HLA-DM, HLA-DO, HLA-DQ, or HLA-DR. In some instances, the donor nucleic acid encoding the CAR is inserted into the HLA gene.

### Certain Vectors

In some instances, compositions, systems, and methods described herein comprise an AAV vector comprising a transgene, wherein the transgene comprises or consists essentially of a nucleic acid encoding an effector protein, a guide nucleic acid, a first promoter driving the expression of the effector protein, a second promoter driving expression of the guide nucleic acid, and a donor nucleic acid, wherein the length of the effector protein is between about 600 amino acid and about 800 amino acids, and wherein the length of the donor nucleic acid is about 50 bp to about 600 bp. In some embodiments, the amino acid sequence of the effector protein is at least about 75%, at least about 80%, at least about 85%, at least about 90%, at least about 95%, at least about 98%, at least about 99% or 100% identical to any one of sequences recited in TABLE 1. In some instances, the amino acid sequence of the effector protein is at least about 75%, at least about 80%, at least about 85%, at least about 90%, at least about 95%, at least about 98%, at least about 99% or 100% identical to a sequence selected from SEQ ID NOS: 46-52, 54-58, 62-84, 87-89, 93-104, 113, 130, 131, 145, and 150. In some instances, the effector protein or corresponding mRNA comprises an NLS and/or a polyA tail, respectively. An NLS is a sequence that tags a protein for import into the cell nucleus. There are many NLS described in the art. The length of the NLS may be about 5 to about 100 amino acids. The length of the NLS may be about 10 amino acids to about 20, about 30, about 40, about 50, or about 60 amino acids. The NLS may be located at the 5′ end of the effector protein. The NLS may be located at the 3′ end of the effector protein. The NLS may be located at an internal site of the effector protein (e.g., between the 5′ and 3′ end of the effector protein, but not at the 5′ or 3′ end of the effector protein). In some instances, the first promoter is an EFS promoter. In some instances, the second promoter is a U6 promoter. In some instances, the donor nucleic acid encodes a chimeric antigen receptor.

In some instances, the guide nucleic acid comprises or consists essentially of a crRNA. In some embodiments, the guide nucleic acid comprises or consists essentially of a sgRNA. In some embodiments, the sgRNA comprises a handle sequence and a spacer sequence. In some embodiments, the handle sequence comprises one or more of a linker, at least a portion of or all of a repeat sequence, and at least a portion of or all of a tracrRNA. In some instances, the length of the guide nucleic acid is less than about 200 linked nucleosides. In some instances, the length of the crRNA/sgRNA is less than about 200, less than about 180, less than about 160, less than about 140, less than about 120, less than about 100, less than about 80, or less than about 60 linked nucleosides.

In some instances, compositions, systems, and methods described herein comprise an AAV vector comprising a transgene, wherein the transgene comprises or consists essentially of a nucleic acid encoding an effector protein, a guide nucleic acid, a first promoter driving the expression of the effector protein, a second promoter driving expression of the guide nucleic acid, and a donor nucleic acid, wherein the length of the effector protein is between about 400 amino acid and about 1400 amino acids, and wherein the length of the donor nucleic acid is about 1 kb to about 1.2 kb, about 1.2 kb to about 1.4 kb, about 1.4 kb to about 1.6 kb, about 1.6 kb to about 1.8 kb, or about 1.8 kb to about 2 kb. In some embodiments, the length of the effector protein is between about 400 amino acid and about 600 amino acids, and wherein the length of the donor nucleic acid is about 1 kb to about 1.2 kb, about 1.2 kb to about 1.4 kb, about 1.4 kb to about 1.6 kb, about 1.6 kb to about 1.8 kb, or about 1.8 kb to about 2 kb. In some instances, the length of the donor nucleic acid is about 1.5 kb to about 2 kb. In some embodiments, the amino acid sequence of the effector protein is at least about 75%, at least about 80%, at least about 85%, at least about 90%, at least about 95%, at least about 98%, at least about 99% or 100% identical to any one of sequences recited in TABLE 1. In some instances, the amino acid sequence of the effector protein is at least about 75%, at least about 80%, at least about 85%, at least about 90%, at least about 95%, at least about 98%, at least about 99% or 100% identical to a sequence selected from SEQ ID NOS: 1-45, 53, 85, 86, 90-92, 105-112, 114-129, 132-144, 146-149, and 151-156. In some instances, the amino acid sequence of the effector protein is at least about 75%, at least about 80%, at least about 85%, at least about 90%, at least about 95%, at least about 98%, at least about 99% or 100% identical to SEQ ID NO: 23. In some instances, the effector protein or corresponding mRNA comprises an NLS and/or a polyA tail, respectively. In some instances, the first promoter is an EFS promoter. In some instances, the second promoter is a U6 promoter. In some instances, the donor nucleic acid encodes a chimeric antigen receptor. In some instances, the guide nucleic acid comprises or consists essentially of a crRNA. In some embodiments, the guide nucleic acid comprises or consists essentially of a sgRNA. In some embodiments, the sgRNA comprises a handle sequence and a spacer sequence. In some embodiments, the handle sequence comprises one or more of a linker, at least a portion of or all of a repeat sequence, and at least a portion of or all of a tracrRNA. In some instances, the length of the guide nucleic acid is less than about 200 linked nucleosides. In some instances, the length of the crRNA/sgRNA is less than about 200, less than about 180, less than about 160, less than about 140, less than about 120, less than about 100, less than about 80, or less than about 60 linked nucleosides.

In some instances, compositions comprise an scAAV vector comprising a transgene, wherein the transgene comprises or consists essentially of a nucleic acid encoding an effector protein, a guide nucleic acid, a first promoter driving the expression of the effector protein, and a second promoter driving expression of the guide nucleic acid, wherein the length of the effector protein is between about 400 amino acids and about 600 amino acids. In some instances, the amino acid sequence of the effector protein is at least about 75%, at least about 80%, at least about 85%, at least about 90%, at least about 95%, at least about 98%, at least about 99% or 100% identical to any one of sequences recited in TABLE 1. In some instances, the amino acid sequence of the effector protein is at least about 75%, at least about 80%, at least about 85%, at least about 90%, at least about 95%, at least about 98%, at least about 99% or 100% identical to a sequence selected from SEQ ID NOS: 1-45, 53, 85, 86, 90-92, 105-112, 114-129, 132-144, 146-149, and 151-156. In some instances, the amino acid sequence of the effector protein is at least about 75%, at least about 80%, at least about 85%, at least about 90%, at least about 95%, at least about 98%, at least about 99% or 100% identical to SEQ ID NO: 23. In some instances, the effector protein or corresponding mRNA comprises an NLS and/or a polyA tail, respectively. In some instances, the first promoter is an EFS promoter. In some instances, the second promoter is a U6 promoter. In some instances, the guide nucleic acid comprises or consists essentially of a crRNA. In some embodiments, the guide nucleic acid comprises or consists essentially of a sgRNA. In some embodiments, the sgRNA comprises a handle sequence and a spacer sequence. In some embodiments, the handle sequence comprises one or more of a linker, at least a portion of or all of a repeat sequence, and at least a portion of or all of a tracrRNA. In some instances, the length of the guide nucleic acid is less than about 200 linked nucleosides. In some instances, the length of the crRNA/sgRNA is less than about 200, less than about 180, less than about 160, less than about 140, less than about 120, less than about 100, less than about 80, or less than about 60 linked nucleosides.

In some embodiments, a guide nucleic acid as described herein is at least about 70%, at least about 75%, at least about 80%, at least about 85%, at least about 90%, at least about 95%, at least about 98%, at least about 99% or 100% identical to any one of sequences recited in TABLE 3, TABLE 4, TABLE 6, and TABLE 7. In some embodiments, a guide nucleic acid as described herein comprises a sequence with at least 8, at least 9, at least 10, at least 11, at least 12, or at least 13 contiguous nucleotides of any one of sequences recited in TABLE 3, TABLE 4, TABLE 6, and TABLE 7. In some embodiments, a guide nucleic acid as described herein is at least about 70%, at least about 75%, at least about 80%, at least about 85%, at least about 90%, at least about 95%, at least about 98%, at least about 99% or 100% identical to a sequence selected from SEQ ID NOS: 202-207 and 329-367. In some embodiments, a guide nucleic acid as described herein comprises a sequence at least about 70%, at least about 75%, at least about 80%, at least about 85%, at least about 90%, at least about 95%, at least about 98%, at least about 99% or 100% identical to UGGGGCAGUUGGUUGCCCUUAGCCUGAGGCAUUUAUUGCACUCGGGAAGUACCAUUUCU CAGAAAUGGUACAUCCAAC (SEQ ID NO: 214). In some embodiments, a guide nucleic acid as described herein comprises a sequence at least about 70%, at least about 75%, at least about 80%, at least about 85%, at least about 90%, at least about 95%, at least about 98%, at least about 99% or 100% identical to UGGUACAUCCAAC (SEQ ID NO: 215). In some embodiments, a guide nucleic acid as described herein comprises a sequence with at least 8, at least 9, at least 10, at least 11, at least 12, or at least 13 contiguous nucleotides of SEQ ID NO: 215. In some embodiments, a composition described herein can comprise CasM.19952 (SEQ ID NO: 23) and a guide nucleic acid comprising any one of SEQ ID NOs: 202-207, 214-215, 224-229 and 231-232. In some embodiments, a composition described herein can comprise CasM.19952 (SEQ ID NO: 23) and a guide nucleic acid comprising SEQ ID NOs: 208-213.

### IV. Effector Proteins

Provided herein are viral vectors encoding a polypeptide (e.g., effector protein) and a guide nucleic acid, and in certain embodiments, are compositions, systems and methods that comprise the viral vectors. An effector protein may also be referred to herein and throughout as a programmable nuclease.

A polypeptide (e.g., effector protein) may be brought into proximity of a target nucleic acid in the presence of a guide nucleic acid when the guide nucleic acid includes a nucleotide sequence that is complementary with a target region in the target nucleic acid. The ability of a polypeptide (e.g., effector protein) to modify a target nucleic acid may be dependent upon the polypeptide being bound to a guide nucleic acid and the guide nucleic acid being hybridized to a target nucleic acid. A polypeptide (e.g., effector protein) may also recognize a protospacer adjacent motif (PAM) sequence present in the target nucleic acid, which may direct the modification activity of the polypeptide. In some embodiments, a given polypeptide (e.g., effector protein) may require a PAM sequence being present in a target nucleic acid for a complex of an effector protein and a guide nucleic acid to hybridize to and modify the target nucleic acid. In some embodiments, a given polypeptide (e.g., effector protein) may not require a PAM sequence being present in a target nucleic acid for the effector protein to modify the target nucleic acid. Accordingly, in some embodiments, a polypeptide (e.g., effector protein) may also recognize a sequence that is at least 1, at least 2, at least 3, at least 4, at least 5, at least 6, at least 7, at least 8, at least 9, or at least 10 nucleotides away from 5′ or 3′ terminus of a PAM sequence present in the target nucleic acid, which may direct the modification activity of the polypeptide.

A polypeptide (e.g., effector protein) may modify a nucleic acid by cis cleavage or trans cleavage. In some embodiments, cis cleavage is a cleavage (hydrolysis of a phosphodiester bond) of a target nucleic acid by a complex of an effector protein and a guide nucleic acid, wherein at least a portion of the guide nucleic acid is hybridized to at least a portion of the target nucleic acid. In some embodiments, cis cleavage may occur within or directly adjacent to the portion of the target nucleic acid that is hybridized to the portion of the guide nucleic acid. In some embodiments, trans cleavage is a cleavage (hydrolysis of a phosphodiester bond) of one or more non-target nucleic acids by an effector protein that is complexed with a guide nucleic acid and a target nucleic acid. In some embodiments, the effector protein may cleave the target nucleic acid as well as non-target nucleic acids

The modification of the target nucleic acid generated by a polypeptide (e.g., effector protein) may, as a non-limiting example, result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization).

A polypeptide (e.g., effector protein) may function as a single protein, including a single protein that is capable of binding to a guide nucleic acid and modifying a target nucleic acid. Alternatively, a polypeptide (e.g., effector protein) may function as part of a multiprotein complex, including, for example, a complex having two or more effector proteins, including two or more of the same effector proteins (e.g., dimer or multimer). A polypeptide (e.g., effector protein), when functioning in a multiprotein complex, may have only one functional activity (e.g., binding to a guide nucleic acid), while polypeptides present in the multiprotein complex are capable of the other functional activity (e.g., modifying a target nucleic acid). In some embodiments, a polypeptide (e.g., effector protein) may be a CRISPR-associated (“Cas”) protein.

In certain embodiments, the polypeptide (e.g., effector protein) described herein can comprise one or more functional domains. In certain embodiments, the polypeptide (e.g., effector protein) described herein can comprise one or more functional domains comprising a protospacer adjacent motif (PAM)-interacting domain, an oligonucleotide-interacting domain, one or more recognition domains, a non-target strand interacting domain, and a RuvC, domain.

A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of a polypeptide (e.g., effector protein) that interacts with target nucleic acid.

In some embodiments, the polypeptides (e.g., effector proteins) comprise a RuvC domain. In some embodiments, the RuvC domain comprises a partial RuvC domain. In some embodiments, a RuvC domain comprises with substrate binding activity, catalytic activity, or both. In some embodiments, the RuvC domain may be defined by a single, contiguous sequence, or a set of RuvC subdomains that are not contiguous with respect to the primary amino acid sequence of the protein. A polypeptide (e.g., effector protein) of the present disclosure may include multiple RuvC subdomains, which may combine to generate a RuvC domain with substrate binding or catalytic activity. For example, a polypeptide (e.g., effector protein) may include three RuvC subdomains (RuvC-I, RuvC-II, and RuvC-III) that are not contiguous with respect to the primary amino acid sequence of the polypeptide (e.g., effector protein) but form a RuvC domain once the protein is produced and folds.

In some embodiments, polypeptides (e.g., effector proteins) comprise one or more recognition domain (REC domain) with a binding affinity for a guide nucleic acid or for a guide nucleic acid-target nucleic acid heteroduplex. In some embodiments, the REC domain is in an α-helical recognition region or lobe. An effector protein may contain at least one REC domain (e.g., REC1, REC2) which generally helps to accommodate and stabilize the guide nucleic acid and target nucleic acid heteroduplex. A polypeptide (e.g., effector protein) may comprise a zinc finger domain. In some embodiments, a polypeptide (e.g., effector protein) does not comprise a zinc finger domain. In some embodiments, a polypeptide (e.g., effector protein) does not comprise an HNH domain.

Provided herein are viral vectors encoding an effector protein and a guide nucleic acid. In general, the effector protein is a Cas effector protein. The effector proteins may be small, which may be beneficial for nucleic acid editing. The small nature of these effector proteins may allow for them to be more easily packaged and delivered with higher efficiency in the context of genome editing.

In some embodiments, effector proteins non-covalently bind to a guide nucleic acid to form a complex that contacts a target nucleic acid, wherein at least a portion of the guide nucleic acid hybridizes to a target region of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is fused to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

An effector protein may be a modified effector protein having reduced modification activity (e.g., a catalytically defective effector protein) or no modification activity (e.g., a catalytically inactive effector protein) relative to the corresponding unmodified effector protein. Accordingly, an effector protein as used herein encompasses a modified effector protein that does not have nuclease activity. Alternatively, an effector protein may be a modified effector protein having increased modification activity and/or increased substrate binding activity (e.g., substrate selectivity, specificity, and/or affinity) relative to the corresponding unmodified effector protein.

In some instances, the length of the effector protein is at least about 300 linked amino acids, at least about 350 linked amino acids, at least about 400 linked amino acid residues, at least about 450 linked amino acids, or at least about 500 linked amino acids. In some instances, the length of the effector protein is at least 400 linked amino acid residues. In some instances, the length of the effector protein is less than about 500, less than about 550, less than about 600, less than about 650, less than about 700, less than about 750, less than about 800, less than about 850, or less than about 900 linked amino acid residues. In some instances, the length of the effector protein is about 400 to about 600 linked amino acid residues. In some instances, the length of the effector protein is about 400 to about 500 linked amino acid residues. In some instances, the length of the effector protein is about 450 to about 550 linked amino acids. In some instances, the length of the effector protein is about 500 to about 600 amino acids, about 600 to about 700 amino acids, about 700 to about 800 amino acids, about 800 to about 900 amino acids. In some instances, the length of the effector protein is about 400 to about 420, about 420 to about 440, about 440 to about 460, about 460 to about 480, about 480 to about 500, about 500 to about 520, about 520 to about 540, about 540 to about 560, about 560 to about 580, about 580 to about 600, about 600 to about 620, about 620 to about 640, about 640 to about 660, about 660 to about 680, about 680 to about 700 linked amino acids, about 700 to about 720, about 720 to about 740, about 740 to about 760, about 760 to about 780, or about 780 to about 800 linked amino acids. In some instances, the length of the effector protein is greater than 800 amino acids. In some instances, the length of the effector protein is less than 900 amino acids. In some instances, the length of the effector protein is less than 1000 amino acids.

In some instances, the Cas effector is a Type V Cas protein. Type V Cas effector proteins may have diverse N-terminal structures and often comprise a conserved single catalytic RuvC-like endonuclease domain that is C-terminal of the N-terminal structures. In some instances, the Cas effector is a Type VI Cas protein. In general, a Type V Cas effector protein comprises a RuvC domain but lacks an HNH domain. In most instances, the RuvC domain of the Type V Cas effector protein comprises three RuvC subdomains. In some instances, the three RuvC subdomains are located within the C-terminal half of the Type V Cas effector protein. In some instances, none of the RuvC subdomains are located at the N terminus of the protein. In some instances, the RuvC subdomains are contiguous. In some instances, there are zero to about 50 amino acids between the first and second RuvC subdomains. In some instances, there are zero to about 50 amino acids between the second and third RuvC subdomains. In some instances, the Cas effector is a Cas14 effector. In some instances, the Cas effector is a Cas12 effector. In some instances, the Cas12 effector is a Cas12a, Cas12b, Cas12c, Cas12d, a Cas12e or a Cas12j effector. In some instances, the Cas effector is a Cas13 effector. In some instances, the Cas13 effector is a Cas13a, a Cas13b, a Cas13c or a Cas13d effector.

In some embodiments, effector proteins comprise a “RuvC-like” domain. Various RuvC-like domains are known in the art and are easily identified using online tools such as InterPro (https://www.ebi.ac.uk/interpro/). For example, a RuvC-like domain may be a domain which shares greater than 5000 identity with a region of TnpB proteins of the IS605 and other related families of transposons.

TABLE 1 provides illustrative amino acid sequences of small effector proteins having a length of less than 900 linked amino acids. In some instances, viral vectors described herein comprise a sequence that encodes an effector protein, wherein the amino acid sequence of the effector protein is at least 650%, at least 70%, at least 75%, at least 80%, at least 85%, at least 90%, at least 95%, at least 97%, or at least 98%, at least 99%, or 100% identical to any one of the sequences recited in TABLE 1.

TABLE 1
Amino Acid Sequences of Exemplary Effector Proteins
Effector Protein Amino Acid sequence
CasM.298706
MAKKGTNRKKMIVKVMKYELKYESGCADFNEMQNELWKLQRQTREVMNRTIQLCYHWSY
VQADYCKQHGCARRDVKPCDVYETNATSLDGYIYQLFKDEYPNFLMANLIATLRKAHQKYD
ALLFDIQEGNSSIPSFKKDQPLIFSKEAIRLPECLSDKRQITLFCFSKPYKSAHPTLDKITFAVRAR
SASEKSIFDHIISGKYALGESQLVYEKKKWFFLLSYKFTPESVDVNPEKVLGVDLGVVNALCAG
SVENPHDSLFIKGTEAIEQIRRLEARKRDLQKQARYPGDGRIGHGTKTRVSPVYQTRDAIARMQ
DTLNHRWSRALIDFACKKGYGTIQMEDLSGIKALESEKPYLKHWTYFDLQSKIIYKAEEKGIRV
VKVNPKCTSRRCSACGYISKENRKNQVEFLCVNCGYHHNADYNAAQNLSIPQIDRLIEKQLKE
QESEENEAGANPK (SEQ ID NO: 1)
CasM.280604
MAKGTLSKVMKYELRYLDGCGDFQNMQKELWTLQRQSREILNRTIQIAYHWDYTDREQFKK
TGQHLDIKAETGYKRLDGYIYDSLKEDVQNFASVNVNATIQKAWAKYKSSKIDVLRGDMSLP
SYKSDQPLVLHAQSMKIFSSDDDDVLQVTLFSNAYKKACNYSNIRFIIGLHDATQRTIIKKVLSG
DWGIGQSQIVYKRPKWFLYLTYNFSPEQHEVNPDKILGVDLGESIAIYASSIGEYGSLRIEGGEIS
AFAKQLEARKRSLQKQAAYCGKGRIGHGTKSRVSDVYKMEDKIANFRNTVNHRYSKMLIDY
ALKHMYGTIQMEDLSGIKKETGFPKFLQHWTYYDLQQKIEAKAKEHGINFIKVDPAFTSQRCS
KCGNIDSENRPSQAVFCCKKCGYKTNADFNAS (SEQ ID NO: 2)
CasM.281060
MNVTKVMRYQLIYQGGGGDFESLQNQLWEFQRQTRAILNKTIQTMYLATANQEKFSEKALY
HDLCAEYPDMISSTVNATLREATKKYRSSVREILAGRMSLPSYKRDHPILLHNQSVALKQGNQ
GSYFATISVFSRKYQQGTPGVKQPSFQLIAKDNTQRTILQRLLSGEYKLGQCQLIYIRPKWFLNV
AYSFTPSEKALDQEKVLGVDLGCVYAIYASSYGNHGIFKISGDEITSFERKQAAIQNRAFKNDL
TRIREIEERRKQKLEQARYCGEGRIGHGVKTRVAPAYQDEGKISRFRETINHRYSKALVDYAEK
NGYGTIQMEDLSGIKSSTGFPKRLQHWTYFDLQQKIKYKAEEQGIKVVKIKPAYTSQRCSRCG
HIDPANRKSQSEFKCIACGFSSNADYNASQNISMRNIEKIIQGKAN (SEQ ID NO: 3)
CasM.284933
MAKGTITKVMKYELRYLGGFSDFHEMQKEVWQLQRQYREILNKTIQIALHWDYVSAQQFGES
GTYLDIREETGYKTLDGYIYNCLKGAYSEMASANLNAAVQKAWKKYKNSKTQVLQGVMSLP
SYKSDQPILIDKGNVKLSAEENNGRAVLTLFSRNYRDTRGLKGNVEFSVLLHDGTQKSIFRNLI
DKTYALGQCQLVYERKKWFLLLTYSFTPAGHALDPEKILGVDLGECYALYASSCYAPGILKIE
GGEIAEYALRLEKRKRSLQQQARYCGEGRIGHGTKTRVGVVYKAEDRIASFRETINHRYSKEL
VDYAVSNGYGTIQMEDLSAIQKDLGFPKRLRHWTYYDLQMKITNKAKEHGIAVVKIDPRYTS
QRCSKCGHIDPANRPRQEEFCCTACGYACNADYNASQNISIKGIEKIIQKMLSAKAD (SEQ ID
NO: 4)
CasM.287908
MSKGMLTKVMKYTLRYVGGCGDFHEMQSILWELQKQTRAVLNKTIQIAFEWDYRSREAFQE
TGEYLDVHAETGYKRLDGYIYNCLKNEYADFAGKNLNAAIQTAWKKYNQSKRDIQTGKMSL
PSYRSNQPLIIHNDNVMISQDMQAAPSVRFTLLSLEYKKAHDLNTNPTFEVLINDGTQRAIFEK
VRSGEYKLGQCMIQYDKKKWFLLLTYSFQPEKLTLDKNKILGVDLGETIVICASSVSERGRFVI
DGGEITRFATQIEARKRSQQHQAAYCGEGRIGHGTKTRVDAVYKTEDRIANFRDTINHRYSRA
LVNYAVKHGFGTIQMEDLSGIKSSDDFPKFLRHWTYYDLQSKIESKAKERGIAVVKVNPRFTS
RRCSKCGYIDEGNRKDQAHFCCLSCGFRANADFNASQNLSIKGIDKIIEKEYNANSKQT (SEQ
ID NO: 5)
CasM.288518
MGKPITKTMKYQIHYIDGCGDFHNMQKELWDLQRIVRQILNKTINESYLWFVRSEQYYRDTG
ENLSVEEQTGYKTLDGHIYNLLKQEYTQKLVSNSLNASIQAAYKKMKDSRRDVMIGTMSLPS
YRSDQPIIIYNKNIKFSSHPEHGFVVDCSLFSDAYKKSQGYEKSVKFQVSVDDNTQRSIFENILT
GNYKHGQCSIVYEKKKWFLLLTYSFVPEETKLDPDKILGVDVGVVYALYASSKGNHGTFKIK
GDEAITFIQRVEARKHSRQLQGTYCGDGRIGHGTKTRVQPVYNERALISNFQDTINHRYSKALI
DYAKKNGYGTIQMEDLSGIKEVQQYPKYLQHWTYYDLQLKIQYKAKEAGIGFVKVTPKYTSQ
RCSHCGNIDEANRPKQDVFRCTVCGYERNADYNASQNLSIKGIDRIIDDQLKQMNKANPKKTE
NA (SEQ ID NO: 6)
CasM.293891
MSGGAITKVMKYDLTYKDGYGNFKDMQEAVWKLIRDTRTILNETIKIAYHWDYLNEKSKRET
GEHLDLLEETGYKRLDGYIYDDLKDRFPDFASSNLNAAIQTAWKKYKQSQKDVYIGKMTLPS
YKSDQPLPINKQSIKIYDEEREHIVELNLFSTKHKKEHGLASNVRFRINLHDNTQHAIYERVLSG
EYTLGQCQLLYDRPKWFFILTYSFKPAQNKLDPDKILGVDMGETCALYASTFGEQGSFVINGG
EVSEYAKREEARKRSLQKQAAVCGEGRIGHGTKTRVSSVYKEQERISNFRDTINHRYSKALIEY
AVKNGCGTIQMEDLSGIRQSTDFPKFLRHWTYYDLQQKIKTKAKETGIAVSMIDPRYTSQRCS
RCGHIDKANRKDQAHFHCLKCGYSCNADFNASQNISIRGIDKIIQKELGAKAKQTD (SEQ ID
NO: 7)
CasM.294270
MKEIAKVMKYQLIYLDGGGDFYELQQTLWDLQRQTREILNKTIQSMYLATATNTAFEENALY
HRFGAEYPMMAALNVNATLRTAKKRYTSTIKETLRGTMSLPSYKRDQPILLHNQTIHLALEDG
QYSALFSVYSEKFQKAHEGVARPRFALMARDGTQRAILDRLLDGSYRLGQSQMTYEQKKWF
LSLTYKFVPEVRELDKSKILGVDLGCVYAIYASSMQQKGIFKISGDEITEFEKRQAAMQNREPV
STLERVEQLEQRRWQKQQQARYCGEGRVGHGTGTRVAPAYRDADKIARFRDTINHRYSKAL
VEYAEKNGFGTIQMEDLSGIKEDTGFPKRLRHWTYFDLQTKIQYKAAERGITVVKIDPQYTSQ
RCSRCGYIDKANRASQEKFLCQSCGFEANADYNASQNISVEKIDKLIAKDKKKLART (SEQ ID
NO: 8)
CasM.294491
MGQVTKVMRYQLIYQDGGGDFYTVQQELWELQRQTREILNKTIQTMYLADANKEKFDNAAE
RTLNRRFCVDHPDMYTKTVTATLRKAKAKYNASQKEILAGRMSLPSYKRDQPILLNPQGFKIE
EESDSFFAAIAVFSDKYKNKHPDVDVKRLRFRLVVKDGTQRAIIRRVISGEYKLGRSQLLYSKK
KWFLNVTYSFEPAEKKVDPDKILGVDLGCVYAIYASSFGSPGVFKISGDEVSSFERKQAAIQNR
SPKSTLERVEKIEERHKQKQQQARYCGEGRIGHGTKTRIAPVYQDEDKIARFRDTVNHRYSKA
LIDYAEKNGYGTIQMEDLSGIKSATGFPKRLKHWTYYDLQTKIEYKAEERGIKVVKIDPRYTSQ
RCSRCGYIDSGNRKSQAEFCCMACGFSCNADYNASQNISIGGIAKIIADKRKEADAK (SEQ ID
NO: 9)
CasM.295047
YLDIREETGYKTLDGYIYNCLKGAYSEMASANLNAAVQKAWKKYKNSKTQVLQGVMSLPSY
KSDQPILIDKGNVKLSAEENNGRAVLTLFSRNYRDTRGLKGNVEFSVLLHDGTQKSIFRNLIDK
TYALGQCQLVYERKKWFLLLTYSFTPAGHALDPEKILGVDLGECYALYASSCYAPGILKIEGG
EIAEYALRLEKRKRSLQQQARYCGEGRIGHGTKTRVGVVYKAEDRIASFRETINHRYSKELVD
YAVSNGYGTIQMEDLSAIQKDLGFPKRLRHWTYYDLQMKITNKAKEHGIAVVKIDPRYTSQR
CSKCGHIDPANRPRQEEFCCTACGYACNADYNASQNISIKGIEKIIQKMLSAKAD (SEQ ID NO:
10)
CasM.299588
MAEKTIVKVMKFELRYIDGAGEFSEMQKHLWELQKQTREVLNKTIQMGYALECKRFAHHDK
TGQWLDDKELTGSKYKAVADYINAELKEDYNIFYSDCRNSTVRKAYKKFKDAKNKIFSGEMS
LPSYRSNQPIIIHNRNVIIRGNAESALVGLKVFSDGFKALHGFPAAVNFKLCVKDGTQRAIIENVI
SEIYKISESQLIYDNKKWFLILAYRFTQKKNDLNPDKILGVDLGVKFAVYASSIGEYGSFRIKGG
EVTEFIKRLEKRKKSLQNQATVCGDGRIGHGTKTRVADVYKARDKISNFQDTINHRYSRAIVD
YARKNGYGTIQLEKLDNSIEKKGDYSPVLVHWTYYDLRTKMEYKAAEYGIKVIAVEPKYTSQ
RCSKCGYISSENRKTQESFECIKCGYKCNADFNASQNLSVRDIDRIIDEYLGANPELT (SEQ ID
NO: 11)
CasM.277328
VVNVAKGALSKVMKFELSYLDGCGDFQNMQKELWTLQRQTREILNRTIQIAYHWDYTDREH
FKKTGQHLDVKSETGYKRLDGYIYDELKETVQNFASVNVNATIQKAWAKYKSSKTDVLRGD
MSLPSYKSDQPLVLHAQSIKLSEDKDGPVLQVTLFSNAHKKACDYSNVRFAFRLHDATQRAIF
KNVLSGEYGLGQSQIVYKRPKWFLYLTYNFSPEQHGLDPDKILGVDLGESIALYASSLGDYGS
LRIEGGEVTAFAKQLEARKRSLQKQAAHCGEGRVGHGTRARVSDVYKAEDKIANFRNTVNHR
YSKKLIEYAIQNRYGTIQMEDLSGIKQDTGFPKFLQHWTYYDLQQKIEAKAKENGINFIKVDPS
YTSQRCSKCGNIDSDNRPSQAVFCCTKCGFRANADFNASQNLSIPEIDKIIKKERGANTK (SEQ
ID NO: 12)
CasM.297894
MAKKGTNRKKMIVKVMKYELKYEKGCADFNEMQNELWKLQRQTREVMNRTVQLCYHWN
YVQADYCKQHGCAHRDVKPCDVYETNATSLDGYIYQLFKDEYPNFLMANLIATLRKAHQKY
DALLPDIQEGNSSIPSFKKDQPLIFSKEAIHLPECLSDKRQITLFCFSKPYKSAHPTLDKITFAVRA
HSASEKSIFDNIINGKYALGTSQLVYEKKKWFFLLSYKFTPESVDVNPEKVLGVDLGVVNALC
AGSVENPHDSLFIKGTEAIEQIRRLEARKRDLQKQARYPGDGRIGHGTKTRVSPVYQTRDAIAR
MQDTLNHRWSRALIDFACKKGYGTIQMEDLSGIKAMESEKPYLKHWTYFDLQSKIIYKAEEK
GIRVVKVNPKCTSRRCSACGYISKENRKNQAEFLCVNCGYHHNADYNAAQNLSIPQIDRLIEK
QLKEQESEESEAGANPK (SEQ ID NO: 13)
CasM.291449
MTERHDNESSKIKAEVSLLNSSVPDFEKKRHVKVLKLHILKPAGDMKWDELGALLRDARYRV
FRLANLAISEAYLDFHKWRSGGNEQPKLKISQLNRNLRSMLEDEVTGKQTKMIKSDRYSKSGA
LPDSIVSPLSMYKLGGLTSKSKWSEVLRGKSSLPTFKLNMAIPVRCDKPGDRRIERTKNGDAEV
ELRICLQPYPRVIIATGRNSLGDGQRAILDRLLDNTKYSEQGYRQRCFEIKEDQRSGKWHLFVT
YDFPAIEPAKNLSRERIVGVDLGAACPLYAAINTGHARLGWKHFSPLAARVRALQNQTIRRRR
QILRGGKVSLSEDSARSGHGRKRKLKPISKLEGKIDRAYTTLNHQLSATVIKFAKDNGAGVVQ
MEDLKGLRETLTGTFLGERWRYEELQRFIRYKADEAGIEIRLVNPQYTSRRCSECGHIHKDFTR
EFRDKSREGNKSVRFLCPDCGFTADPDYNAARNLASLDIAAIIERQLEIQGLRKHDP (SEQ ID
NO: 14)
CasM.297599
MKEKSKTLVKVARLRILKPAGDMKWSELGEMLRTVRYRVFRLANLAVSEAYLGFHMYRTNR
ATEFKAETIGKLSRRLREMLIEEGVDEKDLSRYSQTGAVPDTVAGALGQYKIRGITSPTKWRQ
VVRGQAALPTFRNDMAIPIRCDKQYQRRLEKTEAGEIEVELMICRKPYPRIVLGTADLGPGQRA
ILERLLQNTDNSADGYRQRLFEAKQDTQTKKWWLYVTYDFPRLKEGKLNQEIVVGVDLGFSIP
LYVALNIGHARLGRRHFQALGNRIRSLQRQVLARRRSIQRGGRVNISHSTARSGHGRKRKLLP
TEKLRGRIEKSYSTLNHQLSASVIDFAKNHHAGTIQIEDLANLKEELAGTFIGARWRYHQLQQF
LKYKAEEAGITLNQVNPRYTSRRCSECGFINIDFDRAFRDAGRTEGRVTKFLCPECGYEADPDY
NAARNISILDIDKLIRVQCKKQGLTYDAH (SEQ ID NO: 15)
CasM.286588
MPERPKTVNKVIWFQIHKPAGDMTWKELGNLLREARYRVFRLANLAVSEKYLSFHMWRTGQ
EYKSETIGKLNRRLREMLIEEGVEEESQKRFSATGALPDTVVSTLAKGKLAAITSKSKWKDVV
NGKTSLPTFKLNMAIPVRCDKAEQRRLRRTESGDVELELMICKQPYPRVVLKTGKLKSGQRAI
LDRLVENNDNSKEGYSQRVFEIKQVENNDGSKEWRLYISYTFPKKAVEANADVAVGVDIGFS
VPLVAAVNNGLERLGYNDFRALNERIRSLQRQVLVRRRSMQSGGRDYVSTPTARSGHGRKRK
LLPIQTLRKRWDNAYTTLNHQLSHAVVSFAENHGAATIQIENVKSLKDELRGTFLGQRWRYFE
LQQFLKYKADEVGIELREVNARYTSRRCSECGYINMAFTRQARDKGRVDGKPMEFVCPECGY
KAHPDYNAARNIAMLDIEQKMQVQCKQQGITYADDSEVL (SEQ ID NO: 16)
CasM.286910
MTWPELGNMLRTVRYRVFRLANLAVSEAYLGFHMFRTKRAEEFKAETMGKLSRRLREMLIEE
GVDEKDLSRYSQTGAVPDTVAGALSQYKIRGITSPTKWRQIVRGQVALPTFRNTMSIPVRCDK
LYQRRLEQGDSGEVEVELMICRNPYPRVVLGTGDLNPGQQAILERLLQNTDNSADGYRQRLFE
IKEDVQTRKWWLYVTYDFPKTTGKLNPEIVVGVDLGFSIPLYVALNSGHARLGYLHFKALGER
IKSLQKQVMARRRAIQRGGRVSISHSTARTGHGVKRKLQPTEKLRGRIEKSYSTLNHQLSASVI
DFAKNHHAGVIQIEDLSGLKEQLTGTFIGARWRYHQLQQFLKYKAEEAGITLKQINPRYTSRRC
SECGFINMDFDRAFRDAGRTYGKVTKFLCPECGYEADPDYNAARNIATLDIEKLIRVQCEKHG
LKFDAH (SEQ ID NO: 17)
CasM.292335
VGKEGKRNVKVMKIRILKPCDGMTWNELGQLLRDARYRVFRLANLTVSEAYLNFHLWRTGR
SQEFKKQTIGQLNRQLRNILQQEKYDDEKLNRYSKTGALPDTVCSALWQYKLMAVMKKSKW
SEVIRGKSSLPTFRNDMAIPVRCDKPEQKRIEKTEQGQVEAALQVCVQPYPRVILGTHTLGDGQ
DAILKRLLDNQNQAIGGYRQRSFEIKYDEQKRWWLFITYDFPATEVATDKTIAVGVDLGVSVP
LYAAVNNGPARLGRREFGGLGRRIRDLRNQTDARRRSIQRSGREGQSDDTARAGHGRKRKLL
PIHILEGRLDKAYTTLNHQMSAAVIKFAAEQGAGIIQIENLAGLQDELRGTFIGGRWRYRQLQD
FLKYKTQEMGIELRQVNPKYTSRRCSKCGFIHKDFDRDYRNRHSENGKPAQFVCPNPDCKYES
DPDYNAARNLATLDIEEQIRVQCQKQGLEYDSKKDKNAL (SEQ ID NO: 18)
CasM.293576
MKEKSKTLVKVARLRILKPAGDMTWSELGEMLRTVRYRVFRLANLAVSEAYLGFHMFRTQR
AAEFKAETMGKLSRRLREMLIEEGVDEKELNCYSLTGAVPDTVAGALHQYKIRGITSPTKWRQ
VVRGQAALPTFRNDMSIPIRCDKPYQRRLEKTEAGEVEVELMICRKPYPRIVLGTADVGPGQE
VILERLLQNKDNSSDGYRQRLFEAKQDRQTGKWWLYVTYDFPRPEEGELNPEIVVGVDLGFS
VPLYVAINNGYARLGRRHFQALGNRIRSLQRQVLARRRSIQRGGRVNISHDTARSGHGIKRKL
LPTEKLRGRIEKSYSTLNHQLSASVIDFTKNHHAGTIQIEDLANLKEVLAGTFIGARWRYHQLQ
QFLKYKADEAGITLKEVNPRYTSRRCSECGFIHKDFDRAFRDSGRTDGKVARFVCPECGYGPV
DPDYNAAKNISTLDIEKHIRVQCKKQGLEYEVH (SEQ ID NO: 19)
CasM.294537
MKEKAKTLVKVARLRILKPAGDMTWPELGNMLRTVRYRVFRLANLAVSEAYLGFHMFRTKR
AEEFKAETMGKLSRRLREMLIEEGVDEKDLSRYSQTGAVPDTVAGALSQYKIRGITSPTKWRQ
IVRGQVALPTFRNTMSIPVRCDKLYQRRLEQGDSGEVEVELMICRNPYPRVVLGTGDLNPGQQ
AILERLLQNTDNSADGYRQRLFEIKEDVQTRKWWLYVTYDFPKTTGKLNPEIVVGVDLGFSIP
LYVALNSGHARLGYLHFKALGERIKSLQKQVMARRRAIQRGGRVSISHSTARTGHGVKRKLQ
PTEKLRGRIEKSYSTLNHQLSASVIDFAKNHHAGVIQIEDLSGLKEQLTGTFIGARWRYHQLQQ
FLKYKAEEAGITLKQINPRYTSRRCSECGFINMDFDRAFRDAGRTYGKVTKFLCPECGYEADP
DYNAARNIATLDIEKLIRVQCEKHGLKFDAH (SEQ ID NO: 20)
CasM.298538
MAKKAKTMFKVTNFRILKPAGDMTWKELGQLLRDARYRTFRMANLALSEAYLNFYLLKKGD
LKEYKNVKIGQIAKRLRDMLIEEGVDEEVQNRFSPKVALPAYVYSALDQFKLRGLTSKSNWK
KVLRGQASLPTFRLNMSVPIRCDKPEHRRLEKTENGNVEVDLMICRKPYPRVVLETLKLDGSS
KAILDRLLENEDNSPGNYRQRCFEVKQNPRSNDWWLYVTYEMPVDKDKKLDPKVIVGVDLG
FSVPLYVAINNGHARLGRRHFQALGKRIHNLQNQVLARRRSIQRGGQVNLSHSTSRSGHGRKR
KLQPTEKLQQKINSAYSTLNHQLSSSVIDFANNHKAGTIQIEDLETLKEQLTGTYIGRQWRYYQ
LQQFIEYKAKENSITVKKINPKYTSRRCSMCGHIHADFDRTFRDRSSNKGFVTKFICPECNFEAD
PDYNAAKNISTLDIENKIKLQCKKQKIDY (SEQ ID NO: 21)
CasM.19924
MPKITRKIELLFDRSGLSEEECKEKWRFIYQINDNLYRVANRLVNQLYLADEIDDILRLSDQEYI
ALRKKLANKKLDEATRISLEEQMSQVMKRVNERRSAILQRPQQSFAYSVVTDSDTEGLTAKIL
DVLKQDVLSHYKADTKEVLKGEKSISNYKKGMPIPFAFNDSLRLYKEDGFFYLKWYNGIRFLL
NFGRDASNNQLIVERCLGISKDEISYKACSSSIQIKKKGNHSKIFLLLVVDVPVEQYAQKPNMV
VGVDLGLNVPIYAASNSTLERKAIGSREAFLNQRGAFQRRFRALQRLQTTKGGRGRLHKLEPL
ERVREAERNWVRTQNHLFSREVINFAIDVGASTIQMEKLANFGRDAQGEVREDKKYVLRNWS
YFELQNLIEYKAKRAGIKVKYINPAFTSQTCSECGQLGERDSIHFKCTNPDCPNCGKDIHADYN
GARNIAKSKDYIK (SEQ ID NO: 22)
CasM.19952
MPTITRKIELTLLTEGLSEEQRKEQWGLLYHINDNLYKAANNISSKLYLDDHVSSMVRMKHAE
YLSLLKELARAEKQKTPDADAIAELRKKVAAAEKEMTDQEHAICKYATEMSTQSLSYRFATEL
ETNIFAKILDCLKQGVFATFNSDARDVKRGERAIRNYKKGMPIPFAWDKSLRIEKDNKDFYLR
WYNGLRFLFNFGKDRSNNRLIVERCLKMDADYDGEYKLCNSSIQIAKREGKTKLFLLLVVKIP
QEHVELNKKVVVGVDLGINVPAYVATNITEERKAIGDREHFLNSRMAFQRRYKSLQRLRGTA
GGKGRAKKLEPLERLRKAEHNWVHTQNHLFSREVVDFAVKSHAATIHMEDLSGFGKDNDGN
ADERKEFVLRNWSYYELQNMIAYKAAKYGIKVEKIHPAYTSKTCSWCGQLGFREGVTFICENP
ECKQCGEKVHADYNAARNIANSKDIIKKNE (SEQ ID NO: 23)
CasM.274559
MPTITRKIELTLCTDGLSEEQRKEQWGLLYHINDNLYKAANNISSKLYLDDHVSSMVRMKHAE
YLSLLKELARAEKQKTPDADAIAELKKKVAATEKEMTDQEHAICKYATEMSTQSLSYRFSTEF
ETKIFAKILDCLKQGVFATFNSDAKDVKRGERAIRNYKKGMPIPFAWTDSLRIKKDNKDFYLL
WYNGLRFLFNFGKDRSNNRLIVERCLKMDADYDGEYKLCNSSIQIAKREGKVKLFLLLVVSIP
KEHVELNKKVVVSVDLGINVPAYVATNITEERKAIGDREHFLNSRMAFQRRYKSLQRLKGTTG
GKGRTKKLEPLERLRKAEHNWVHTQNHLFSREVVDFAVKTHAATIHMEDLSGFGKDNDGNA
DERKEFVLRNWSYYELQNMISYKAAKYGIKVEKIRPAYTSKTCSWCGQHGFREGVTFICENPA
CKQCGEKVHADYNAARNIANSKEIIKKNE (SEQ ID NO: 24)
CasM.286251
MPTITRKIELTLCTEGLSDQERKDQWNLLYHINDNLYRAANNISSKLYLDDHVGSMVRLKHAE
YLSLLRALEKAKKQKAPDEEVIAELSQQVATAEQEMDEQAKAICQYATEMSTQTLSYRFATEL
ETNIFGQILTCLRQGVFSTENSDARDVKRGERSIRTYKKGMPIPFPWNDSLRIGFEDGEFYLRW
YNGLRFRFDFGKDRSNNCLIVQRCMKMDKDYEGDYKLCNSSIQMVKREGKPKFFLLLVVNIP
QERVELNKNIVVGVDLGINAPAYVATNTTPERKQIGDREHFLNERMAFQRRFKSLQRLKGTTG
GRGRAKKLEPLERLRKAEQNWVHTQNHLFSREVIDFAVKARAATIHMEDLSGFGKDNDGNA
DERKEFVLRNWSYYELQNMITYKAAKYGIKVEKIRPAYTSKTCSWCGHQGFREGITFICENPE
CKKFGEKEHADYNAARNIANSKEIIKNNEE (SEQ ID NO: 25)
CasM.288480
MPTITRKIELTLLTEGLSEEQRKEQWGLLYHINDNLYKAANNISSKLYLDDHVSTMVRMKHAE
YLSLLRELARAEKQKKPDVDAIAELREKVTAAEKEMSDQERAICTYATEMSTQSLSYRFATEIE
TNIFAKILDCLKQGVFATFNSDARDVKRGERAIRNYKKGMPIPFAWDKSLRIEKDNKDFYLRW
YNGLRFLFNFGKDRSNNRLIVERCLKMDADYDGEYKLCNSSIQIVKREGKVKLFLLLVVSIPQE
HVELNKKIVVGVDLGINVPAYVATNITEERKAIGDREHFLNSRMAFQRRYKSLQRLKGTAGGK
GRTKKLEPLERLRKAEHNWVHTQNHLFSREVVDFAVKSHAATIHMEDLSGFGKDNDGNADE
RKEFVLRNWSYYELQNMIAYKAAKYGIKVERIRPAYTSKTCSWCGQLGFREGVTFICENPECK
QCGEKVHADYNAARNIANSKDIIKKNE (SEQ ID NO: 26)
CasM.288668
MPTMTRKIELKLCTEGLSDEERKAQLGLLYHINDNLYKAANNISSKLYLDDHVSSMVRLKHAE
YLSLLNEFEKAKKKGDEEQIVELSLRVAAAEKELTDQELAICKYATEMSTDTLAYRFANEIEIN
VFGQILACLKQGIHSTFKKDAADVKRGERAIRNFKKGMPIPFPWSKSIRIENEGSDFYLRWYNG
LRFRFDFGKDRSNNRLIVSRCLNLDPDFEDEYKLSNSSLQMVKRDGRPKLFLLLVVNIPQENVE
LNKKIVVGVDLGINSPAYVATNITMERQRIGSRDTFLNARMAIQRRFQSLQKLQNTAGGRGRK
KKLEPLERLKETERNWVRTQNHLFSRDVVQFAVKTRAATIHMEDLSGFGKDDDGNADEKKEF
VLRNWSYYELQTMIKYKAAKYGIKVEKIRPAYTSRTCSWCGHEGDRKGETFICENPECEKYG
KKENADYNAARNIANSTDIIK (SEQ ID NO: 27)
CasM.289206
MPTITRKIELTLCTEGLSDEQRKEQWGLLYHINDNLYKAANNISSKLYLDEHVSSMVRMKHAE
YLSLLKELARAEKQQTPDEGLIAELSRKLSAAEKEMADQELAICKYATEMSTQTLSYNFAKEIE
TNIFGQILTCLRQGVYATFNSDAKDVKRGERAIRNYKKGMPIPFPWNNSLKIESDSGEFYLRWY
NGLRFLLTFGKDRSNNRMIVNRCMKMDEDFEGEYKLCNSSIQLAKRDGKPKLFLLLVVNIPQE
HVKLNKKIVVGVDLGVNVPAYVATNITEERKAIGDREHFLNTRMAFQRRYKSLQRLKGTAGG
KGRTKKLEPLERLRDAERNWVHTQNHLFSREVVNFAVQARAATIHMEDLSGFGKDKDGNAD
EKKEFVLRNWSFYELQNMIAYKSAKYGIKVVKIRPAYTSKTCSWCGQQGDRKSTTFICENPKC
KHYGESIHADYNAARNIANSNDIVKENE (SEQ ID NO: 28)
CasM.290598
MPKITRKIEMTLCTEGLSDEQRKEQWGLLYHINDNLYKAANNISTKLYLDEHVSSMVRMKHA
DYLSLLKELAKAEKKSPDEDLIAELREKLAAAEQEMTDQELAICKYATEMSTQTLAYKFATEI
EINVFGQILACLKQAAQSNFKSDAKDVKRGERAIRNYKKGMPIPFPWNDNIRIDADGDEFYLR
WYNGLRFHLTFGKDKSNNRMIVKRCLKMDKDFEGEYKLCNSSIQMVKRDGKPKLFLLLVVNI
PQEHVELNKNVVVGVDLGVNVPAYVATNITEERKAIGEREHFLNTRMQIQRRYKSLQRLKAT
AGGKGRTKKLEPLERLRKAEHNWVHTQNHLFSREVVNFAVQTHAATIHMEDLSGFGKDDDG
NADEQKEFVLRNWSFYELQNMIAYKAAKYGIKVEKVKPAYTSKTCSWCGQLGFRQGVTFICE
NPACKQCGEKVHADYNAARNIANSKDIIKKNE (SEQ ID NO: 29)
CasM.290816
MPTITRKIELHLCTDGLTDEQQKAQRLLLYHINDNLYKAANNVSSKLYLDEHVSSMVRLKHDE
YLSLSRELARAEKKHDDELTTELRGKLAAAEREMTDQELAICKYATEMSTQSLSYRLVTELET
KIFAKILDCLKQGVYATFNSDARDVKRGERAIRNYKKGMPIPFAWNDSVRIEYDEKEKDFYLR
WYNDIRFKFHFGRDRSNNRLIVSRCLKLDKDYEGDYQLCNSSIQIVKRDGSTKFFLLLVVKIPQ
EHVELNKRIVVGVDLGINYPAYVATNCTEERMYIGDREHFLNTRMQFQRRYKSLQKLKGTAG
GKGRSKKLEPLERLRNAERNWVHTQNHLFSLKVVNFAVQTHAATIHLEDLSGFGKDDDGNA
DERKEFVLRNWSYYELQSMIEYKAKKYGIKVEKIRPAYTSQTCSWCGQRGFRQGVTFICENPE
CKKCGEKENADYNAARNIANSKDVIKDKNE (SEQ ID NO: 30)
CasM.295071
TPFVLYFQNYSLSLRQHITLYSMPTITRKIELTLCTEGLSDQERKDQWNLLYHINDNLYRAANN
ISSKLYLDDHVGSMVRLKHAEYLSLLRAMEKAKKQKAPDEEVIAELSQQVAAAEQEMDEQA
KAICQYATEMSTQTLSYRFATELETNIFGQILTCLRQGVFSTFNSDARDVKRGERSIRTYKKGM
PIPFPWNDSLRIGFEDGEFYLRWYNGLRFRFDFGKDRSNNRLIVQRCMKMDKDYEGDYKLCN
SSIQMVKREGKPKFFLLLVVNIPQERVELNKNIVVGVDLGINAPAYVATNTTPERKQIGDREHF
LNERMAFQRRFKSLQRLKGTTGGRGRAKKLEPLERLRKAEQNWVHTQNHLFSREVIDFAVKA
RAATIHMEDLSGFGKDRDGNADERKEFVLRNWSYYELQNMITYKAAKYGIKVEKIRPAYTSK
TCSWCGHQGFREGITFICENPECKKFGEKEHADYNAARNIANSKEIIKNNEE (SEQ ID NO: 31)
CasM.295231
MPTITRKIELHLCTEELSDEQQKAQRLLLYHINDNLYKAANNVSSKLYLDEHVSSMVRLKHDE
YLSLLRELARAEKKADDELATQLREKLVAAEREMTDQELAICKYATEMSTQSLSYRFVTELET
KIFAKILDCLKQGVYATFNSDSRDVKRGERAIRNYKKGMPIPFAWDKSVRIEYEEKEKDFFLR
WYNDIRFKFHFGRDRSNNRLIVSRCMKLDKDYEGDYQLCNSSIQIVKRDGSTKYFLLLVVKIP
QEHVELNKKIVVGVDLGINYPAFAATNCTEERMSIGDREHFLNTRMQFQRRFKSLQRLKGTTG
GKGRNKKLEPLERLRKAEHNWVHTQNHLFSLKVVNFAVQAHAATIHLEDLSGFGKDDDGNA
DERKEFVLRNWSYYELQNMIKYKAKKFGIQVEKIRPAYTSQTCSWCGQRGFRQGITFICENPE
CKKCGEKENADYNAARNIANSKDIIKDKDE (SEQ ID NO: 32)
CasM.292139
MPIITRKIELHISKEGLSAEDYKAQWQYLRQINDNLYMAANRVSSHCFLNDEYKYRLCLQIPDY
IDIEKQLKDSKRARLSKEELGQLKKRKKELENTVKGRFQDEFEKNSLYTIISNEFGEIIPGQILTC
LRQCVQSKYNRAKEELEKGERAISTYKKGMPIPFPINKSIRLQKQGEDFVLKWYNKIVFKLHFG
RDRSNNRVIVERLIQSALNDKQKGEDYVMNNSSIQLVEKDKMTKIFLLLSMDIPTQKRKLDSEL
VLGVDLGLNFPLYYATNQSANIHDHIGDKDIFLKERMVFQRRFKELQRLQCTQGGRGRKKKL
EPLEKLRDKERNWVRTKNHIFSREVIKVALHLGAGTIHLENLHNFGKDGNGELKNSKKFVFRN
WSYFELQSMIEYKAKMEGITVKYVNPAYTSQTCSVCGMIGERKEQAVFRCMNSSCLEYGKEV
NADFNAARNIAKAKM (SEQ ID NO: 33)
CasM.279423
MPTITRKIELTLCTDGLSDDLRKDQWQLLYHINDNLYKAANNISSKLYLDEHVASMVRLKHAE
YLGLIKELAKARKRADDEAVRDLCSKLAVAEQEMNEQAKAICDYATEMSTQTLSYNFAKEIE
TNIFGQILTCLRQGVLLNFNSDARDVKRGERAIRNYKKGMPIPFPWNDTIKIVSEGDEFYLRWF
SGLRFHLNFGKDRSNNRMIVRRCLKMEQDFDEEYKISNSSIQVAKRDGKQKLFLLLVVQIPQE
QVVLNKKIVVGVDLGVNVPAYVATNITEERKAIGDREHFLNTRMQFQRRYKSLQRLKTTEGG
RGRAKKLEPLERLRKAEHNWVHTQNHLFSREVVNFALQTQAATINMEDLSGFGKDNDGNAD
ECKEFVLRNWSYYELQNMIVYKASKYGIRVQKIRPAYTSKTCSWCGHMGFREGVTFICENPDC
KQFGEKVHADYNAARNIANSKEIIKNDE (SEQ ID NO: 34)
CasM.20054
MSKTVTKTVKIALICEHTNKYGEKVDYKDINKLLWKLQKQTRELKNKTIQLCWEYNNFSCDY
YKEHHEYPNMEDILKYKRINGFVENKLKTVNDLYSSNCSTTILSTCNEFQNYRSEFLKGTRSIN
SYKSDQPLDLHKGAIKLEHDGKDFYVSLKLLKRSAFNAMEFKGSDIRFKLNVKDKDKSTLKIL
ESCYDKIYSISASKMTYDRKAGKWFLLLAYSFTPAKTENLDPEKILGVDLGIKIPICASVYGDLD
RLTIEGGKIEEFRRRVEARKRSLQKQGKQCGDGRIGHGTKKRIKPITDIGDKIARFRDTENHIYS
RYLIEYAVKKGCGTIQMEKLEGITREKDIFLKNWTYFDLQKKIEYKAKEKGIKVVYIEPAYTSK
RCSSCGFIDTDNRLDQAHFKCLKCGFNENADYNASQNIGIKNIDKIIKEEHKSASDKLTSE (SEQ
ID NO: 35)
CasM.282673
VIILTKVVKLYLISEQINKEGQKIDYQRINSILWDLQKQTRDIKNRTVQLCWEWMNFSSDYCKT
QEEYPKERDILGYTLEGYVYDYFKTGYDLYTGNISTSSREVCSSFKNVKKEILKGERSILSYKA
NQPLDLHKKAISLEYDNFNFFVKLKLLNRTGKKKYDITEDINFKIQVNDKSTRTILERCYDKEY
KISGSKLIYEKKKKLWRLNLCYSFENSQVETLEKDKILGIDLGIVYPLMASIYGEYDRFSIKGGEI
EEFRRRTEARKRSILQQTKYCGDGRIGHGRNKRTQPAYKINDKIARFRDTANHKYSRALIEYA
VKKNCGIIQMENLTGISDNTDCFLKDWSYYDLQTKIENKAKEMGIKVVYIKAQYTSQRCSRCG
YIDVNNRIRQALFKCQNCGYETNADYNASQNIGMYDIENIIEETLKIQSANVKQS (SEQ ID NO:
36)
CasM.282952
MTKVTKVYLISEQIDKDGNKIDFKKISELLWNLQMQTRDIKNKCVQLCWEWLNFSSDYYKKS
EEYPKEKDTLGYTLSGFVYDRIKNGSDLYSSNLSTSSRDTCTAFSNYKKEMLKGERSVLSFKA
NQPLDIHNKAIKLSYENGNFFVALKMLNRAGKEKYGIKDDLRFRMQVRDKSVRTILERLMND
EYKVSASKLMYDKKKKLWKLNLCYSFDNHVISTLDTEKIMGVDLGVVYPIMASVNGDYARFS
IKGGEIEAFRSRVEARRRSLLNQSRYCGDGRIGHGRKKRTEPATQIADKIARFRDTTNHKYSRA
LIDYAIKNGCGTIQMEKLTGITSSAEHFLKEWSYFDLQTKIESKAKEAGIKVVYINPKFTSQRCN
KCGYIHTDNRPVQARFCCQKCGYEENADYNASQNIGTKHIDVIIEETLKMQCEPETPTE (SEQ
ID NO: 37)
CasM.283262
MNKVVKLALICEQSDKDNSPVDYKKINEILWELQKQTREIKNKAIQYCWEYNNFSSDYYKKF
NEYPKEKDILSYTLVGFVNDKFKTGNDLYSGNCSTTVRNACTEFKNSKKELIKGSRSIINYRSN
QPLDIHNKCIRIEFENNCFYTYLKLLNRPAFKKYNFANTEIKFKILVRDNSTKTILERCISNEYEIA
ASKLLYDQKKKCWFLNLVYAFEIKSNNSLDPNKILGVDLGIHYPICASVYGSLDRFTIDGGEID
EFRRRVESRKISMLKQGKNCGDGRIGHGIKARNKPVYNIEDKIARFRDTANHKYSRALIEYAV
KHTCGTIQMEDLTGITDIANRFLKNWSYYDLQTKIEYKAKEAGINIVYIDPKNTSRRCSKCGYI
DKENRETQSRFICLKCGFKENADYNASQNIGIKDIDKLIKEDVH (SEQ ID NO: 38)
CasM.284833
VTLLVKVVKIYLISEQFDKAGNQIDYKEVNKILWELQKQTREAKNKTVQLLWEWNNFSSDYV
KASGIYPKAKDIFGYSSVHGQANKELRTKLALNSSNLSTTTMDVCKIFNTYKKEVWEGKRSVP
SYKSDQPLDLHKESIKLIYENNEFYVRLALLKKAEFAKYGFKDGFRFKMQVKDNSTKTILERCF
DEVYKINASKLLYDQKKKKWKLNLSYSFDNKNISELDKEKILGVDVGVNCPLVASVFGDRDR
FIIKGGEIEKFRKSVEARRRSMLEQTKYCGDGRIGHGRKKRTEPALNIGDKIARFRDTTNHKYS
RALIEYAVKKGCGTIQMEKLTGITSKSDRFLKDWTYYDLQTKIENKAKEVGINVVYIAPKYTS
QRCSKCGYIHKDNRPNQAKFRCLECDFESNADYNASQNIGIKNIDKIIEKDLQKQESEVQVNEN
K (SEQ ID NO: 39)
CasM.287700
MNKVVKLALICEQSDKNNSPVDYKKVNEILWELQKQTREIKNKTIQYCWEYYNFSSDYYKKF
NKYPKEKDILSYTLWGFINDKFKTGNDLYSGNCSATTKKVIKEFKNSKKELIRGSRSIINYKSNQ
PLNIHNKCIHLQFKNNNFYVSINLLNRRSFKKYNFANTAIKFKILVRDNSTKAILERCISNEYKIS
ESQLIYNKKKKCWFLNLSYAFEIKSNNSLDPNKILGVDLGIHYPICASVYGSLDRFTIDGGEIDE
FRRRVESRKISMLKQGKNCGDGRIGHGIKARNKPVYNIEDKIARFRDTANHKYSRALIEYAVK
NNCGTIQMEDLTGITDNANRFLKNWSYYDLQTKIEYKAKEASINVVYINPENTSRRCSKCGYID
KENRKTQSSFICLKCGFKENADYNASQNISIKDIDKLIKEDVH (SEQ ID NO: 40)
CasM.291507
VTLLVKVVKIHLISEQFDKAGNRIDYEEVNKILWELQKQTREAKNKTVQLLWEWNNFSSDYV
KASGIYPKAKDIFGYSSVHGQANKELRTKLALNSSNLSTTTMDVCKNFNTYKKEVWKGKRSV
PSYKSDQPLDLHKDSIKLIYENNQFYVRLALLKKAEFAKYGFKDGFHFKMQVKDNSTKTILER
CFDEVYKINASKLLYDQKKKKWKLNLSYSFDNKNISELDKEKILGVDVGVSYPLVASVFGDR
DRFKIKGGEIEKFRKSVEARRRSMLEQTKYCGDGRIGHGRKKRTEPALNIGDKIARFRDTTNHK
YSRALIEYAVKKGCGTIQMEKLTGITSKADRFLKDWTYYDLQTKIENKAKEVGINVVYIAPKY
TSQRCSKCGYIHKDNRPNQAKFRCLECDFESNADYNASQNIGIKNIDKIIEKDLQKQESEVQVN
ENK (SEQ ID NO: 41)
CasM.293410
LIWKDALGGIILTKIVKLYLISEQIDKDGNRVDYKEINSILWNLQKQTRDIKNKTVQLCWEWM
NFSSDYYKKNELYPNEKEILNLTLRGYAYDHFKQGYDLYSSNISVLTEAVCGAFKNAKKEML
NGEKSVLSYKAEQPLDIHKKCIKLEYDKNFYVKLKMLNKAGKKKYGIEDDLNFKIQVEDKST
RTILERCIDGEYVVSGSKLIYDKKKKLWKLNLCYSFKANEIESLDKNKILGIDLGIACPLMASVN
GEFDRFSIKGGEIETFRKRIEARKRSVLHQTKYCGDGRIGHGRNKRTEPAYKINDKIARFRDTA
NHKYSRALIDYAIRKNCGMIQMENLTGISDKKEHFLKEWSYYDLQTKIENKAKEKGIKIVYINP
EYTSQRCSKCGYIDANNRELRAVFKCQKCGFEADADYNASQNIGIKNIEDIIENTLKISSANEKQ
TKNT (SEQ ID NO: 42)
CasM.295105
VFYSTFLCYILTKYIDFSANECYNINTSSEVKQLMNKVVKLALICEQSDKDNSPVDYKKINEIL
WELQKQTREIKNKAIQYCWEYNNFSSDYYKKFNEYPKEKDILSYTLVGFVNDKFKTGNDLYS
GNCSTTVRNACTEFKNSKKELIKGSRSIINYRSNQPLDIHNKCIRIEFENNCFYTYLKLLNRPAFK
KYNFANTEIKFKILVRDNSTKTILERCISNEYEIAASKLLYDQKKKCWFLNLVYAFEIKSNNSLD
PNKILGVDLGIHYPICASVYGSLDRFTIDGGEIDEFRRRVESRKISMLKQGKNCGDGRIGHGIKA
RNKPVYNIEDKIARFRDTANHKYSRALIEYAVKHTCGTIQMEDLTGITDIANRFLKNWSYYDL
QTKIEYKAKEAGINIVYIDPKNTSRRCSKCGYIDKENRETQSRFICLKCGFKENADYNASQNIGI
KDIDKLIKEDVH (SEQ ID NO: 43)
CasM.295187
LISEQIDKDGNRVDYKEINSILWNLQKQTRDIKNKTVQLCWEWMNFSSDYYKKNELYPNEKEI
LNLTLRGYAYDHFKQGYDLYSSNISVLTEAVCGAFKNAKKEMLNGEKSVLSYKAEQPLDIHK
KCIKLEYDKNFYVKLKMLNKAGKKKYGIEDDLNFKIQVEDKSTRTILERCIDGEYVVSGSKLIY
DKKKKLWKLNLCYSFKANEIESLDKNKILGIDLGIACPLMASVNGEFDRFSIKGGEIETFRKRIE
ARKRSVLHQTKYCGDGRIGHGRNKRTEPAYKINDKIARFRDTANHKYSRALIDYAIRKNCGMI
QMENLTGISDNKEHFLKEWSYYDLQTKIENKAKEKGIKIVYINPEYTSQRCSKCGYIDANNREL
RAVFKCQNCGFEADADYNASQNIGIKNIEDIIENTLKISSANEKQTKNT (SEQ ID NO: 44)
CasM.295929
LVKVVKIYLISEQVDEQGKDVDYNTICGVLWDLQWETREIKNKTVQLCWEWSGFSSDYYKK
YGEYPKEKNLLDYTMGGFVYDKLKSKYHLYTANLSTTSQNTCGIFRTYKVDFVKGNRSVLSF
KADQPLDVHKKSISIDRIDDNYFVKLKLLNKSGIQKYGIRDDFHFRMLVKDNSTKTILERCVGG
DYKAAASKIIYDKKKKMWCLNLSYEFDVNTAKDLNKNRILGIDIGIVYPVVASVNGELDRFVI
QGGEIETFRRRVENRKKSLLKQTKYCGDGRIGHGRNKRTEPVDIISDQIARFRNTANHKYSRAV
IDYAVRKQCGTIQMENLKGITDKSDRFLKNWSYYDLQQKIEYKAKEKGINVVFINPKYTSQRC
SRCGYIDSANRPKLPNQSKFLCIKCGFTENADYNASQNIALYNIEKLIDAEA (SEQ ID NO: 45)
CasΦ.1
MADTPTLFTQFLRHHLPGQRFRKDILKQAGRILANKGEDATIAFLRGKSEESPPDFQPPVKCPII
ACSRPLTEWPIYQASVAIQGYVYGQSLAEFEASDPGCSKDGLLGWFDKTGVCTDYFSVQGLN
LIFQNARKRYIGVQTKVTNRNEKRHKKLKRINAKRIAEGLPELTSDEPESALDETGHLIDPPGL
NTNIYCYQQVSPKPLALSEVNQLPTAYAGYSTSGDDPIQPMVTKDRLSISKGQPGYIPEHQRAL
LSQKKHRRMRGYGLKARALLVIVRIQDDWAVIDLRSLLRNAYWRRIVQTKEPSTITKLLKLVT
GDPVLDATRMVATFTYKPGIVQVRSAKCLKNKQGSKLFSERYLNETVSVTSIDLGSNNLVAV
ATYRLVNGNTPELLQRFTLPSHLVKDFERYKQAHDTLEDSIQKTAVASLPQGQQTEIRMWSM
YGFREAQERVCQELGLADGSIPWNVMTATSTILTDLFLARGGDPKKCMFTSEPKKKKNSKQV
LYKIRDRAWAKMYRTLLSKETREAWNKALWGLKRGSPDYARLSKRKEELARRCVNYTISTA
EKRAQCGRTIVALEDLNIGFFHGRGKQEPGWVGLFTRKKENRWLMQALHKAFLELAHHRGY
HVIEVNPAYTSQTCPVCRHCDPDNRDQHNREAFHCIGCGFRGNADLDVATHNIAMVAITGES
LKRARGSVASKTPQPLAAE (SEQ ID NO: 46)
CasΦ.2
MPKPAVESEFSKVLKKHFPGERFRSSYMKRGGKILAAQGEEAVVAYLQGKSEEEPPNFQPPAK
CHVVTKSRDFAEWPIMKASEAIQRYIYALSTTERAACKPGKSSESHAAWFAATGVSNHGYSH
VQGLNLIFDHTLGRYDGVLKKVQLRNEKARARLESINASRADEGLPEIKAEEEEVATNETGHL
LQPPGINPSFYVYQTISPQAYRPRDEIVLPPEYAGYVRDPNAPIPLGVVRNRCDIQKGCPGYIPE
WQREAGTAISPKTGKAVTVPGLSPKKNKRMRRYWRSEKEKAQDALLVTVRIGTDWVVIDVR
GLLRNARWRTIAPKDISLNALLDLFTGDPVIDVRRNIVTFTYTLDACGTYARKWTLKGKQTKA
TLDKLTATQTVALVAIDLGQTNPISAGISRVTQENGALQCEPLDRFTLPDDLLKDISAYRIAWD
RNEEELRARSVEALPEAQQAEVRALDGVSKETARTQLCADFGLDPKRLPWDKMSSNTTFISE
ALLSNSVSRDQVFFTPAPKKGAKKKAPVEVMRKDRTWARAYKPRLSVEAQKLKNEALWAL
KRTSPEYLKLSRRKEELCRRSINYVIEKTRRRTQCQIVIPVIEDLNVRFFHGSGKRLPGWDNFFT
AKKENRWFIQGLHKAFSDLRTHRSFYVFEVRPERTSITCPKCGHCEVGNRDGEAFQCLSCGKT
CNADLDVATHNLTQVALTGKTMPKREEPRDAQGTAPARKTKKASKSKAPPAEREDQTPAQE
PSQTS (SEQ ID NO: 47)
CasΦ.3
MYILEMADLKSEPSLLAKLLRDRFPGKYWLPKYWKLAEKKRLTGGEEAACEYMADKQLDSP
PPNFRPPARCVILAKSRPFEDWPVHRVASKAQSFVIGLSEQGFAALRAAPPSTADARRDWLRS
HGASEDDLMALEAQLLETIMGNAISLHGGVLKKIDNANVKAAKRLSGRNEARLNKGLQELPP
EQEGSAYGADGLLVNPPGLNLNIYCRKSCCPKPVKNTARFVGHYPGYLRDSDSILISGTMDRL
TIIEGMPGHIPAWQREQGLVKPGGRRRRLSGSESNMRQKVDPSTGPRRSTRSGTVNRSNQRTG
RNGDPLLVEIRMKEDWVLLDARGLLRNLRWRESKRGLSCDHEDLSLSGLLALFSGDPVIDPV
RNEVVFLYGEGIIPVRSTKPVGTRQSKKLLERQASMGPLTLISCDLGQTNLIAGRASAISLTHGS
LGVRSSVRIELDPEIIKSFERLRKDADRLETEILTAAKETLSDEQRGEVNSHEKDSPQTAKASLC
RELGLHPPSLPWGQMGPSTTFIADMLISHGRDDDAFLSHGEFPTLEKRKKFDKRFCLESRPLLS
SETRKALNESLWEVKRTSSEYARLSQRKKEMARRAVNFVVEISRRKTGLSNVIVNIEDLNVRIF
HGGGKQAPGWDGFFRPKSENRWFIQAIHKAFSDLAAHHGIPVIESDPQRTSMTCPECGHCDSK
NRNGVRFLCKGCGASMDADFDAACRNLERVALTGKPMPKPSTSCERLLSATTGKVCSDHSLS
HDAIEKAS (SEQ ID NO: 48)
CasQ.4
MEKEITELTKIRREFPNKKFSSTDMKKAGKLLKAEGPDAVRDFLNSCQEIIGDFKPPVKTNIVSI
SRPFEEWPVSMVGRAIQEYYFSLTKEELESVHPGTSSEDHKSFFNITGLSNYNYTSVQGLNLIF
KNAKAIYDGTLVKANNKNKKLEKKFNEINHKRSLEGLPIITPDFEEPFDENGHLNNPPGINRNI
YGYQGCAAKVFVPSKHKMVSLPKEYEGYNRDPNLSLAGFRNRLEIPEGEPGHVPWFQRMDIP
EGQIGHVNKIQRFNFVHGKNSGKVKFSDKTGRVKRYHHSKYKDATKPYKFLEESKKVSALDS
ILAIITIGDDWVVFDIRGLYRNVFYRELAQKGLTAVQLLDLFTGDPVIDPKKGVVTFSYKEGVV
PVFSQKIVPRFKSRDTLEKLTSQGPVALLSVDLGQNEPVAARVCSLKNINDKITLDNSCRISFLD
DYKKQIKDYRDSLDELEIKIRLEAINSLETNQQVEIRDLDVFSADRAKANTVDMFDIDPNLISW
DSMSDARVSTQISDLYLKNGGDESRVYFEINNKRIKRSDYNISQLVRPKLSDSTRKNLNDSIWK
LKRTSEEYLKLSKRKLELSRAVVNYTIRQSKLLSGINDIVIILEDLDVKKKFNGRGIRDIGWDNF
FSSRKENRWFIPAFHKAFSELSSNRGLCVIEVNPAWTSATCPDCGFCSKENRDGINFTCRKCGV
SYHADIDVATLNIARVAVLGKPMSGPADRERLGDTKKPRVARSRKTMKRKDISNSTVEAMVT
A (SEQ ID NO: 49)
CasΦ.5
MDMLDTETNYATETPAQQQDYSPKPPKKAQRAPKGFSKKARPEKKPPKPITLFTQKHFSGVRF
LKRVIRDASKILKLSESRTITFLEQAIERDGSAPPDVTPPVHNTIMAVTRPFEEWPEVILSKALQK
HCYALTKKIKIKTWPKKGPGKKCLAAWSARTKIPLIPGQVQATNGLFDRIGSIYDGVEKKVTN
RNANKKLEYDEAIKEGRNPAVPEYETAYNIDGTLINKPGYNPNLYITQSRTPRLITEADRPLVE
KILWQMVEKKTQSRNQARRARLEKAAHLQGLPVPKFVPEKVDRSQKIEIRIIDPLDKIEPYMPQ
DRMAIKASQDGHVPYWQRPFLSKRRNRRVRAGWGKQVSSIQAWLTGALLVIVRLGNEAFLA
DIRGALRNAQWRKLLKPDATYQSLFNLFTGDPVVNTRINHLTMAYREGVVNIVKSRSFKGRQ
TREHLLTLLGQGKTVAGVSFDLGQKHAAGLLAAHFGLGEDGNPVFTPIQACFLPQRYLDSLT
NYRNRYDALTLDMRRQSLLALTPAQQQEFADAQRDPGGQAKRACCLKLNLNPDEIRWDLVS
GISTMISDLYIERGGDPRDVHQQVETKPKGKRKSEIRILKIRDGKWAYDFRPKIADETRKAQRE
QLWKLQKASSEFERLSRYKINIARAIANWALQWGRELSGCDIVIPVLEDLNVGSKFFDGKGK
WLLGWDNRFTPKKENRWFIKVLHKAVAELAPHRGVPVYEVMPHRTSMTCPACHYCHPTNRE
GDRFECQSCHVVKNTDRDVAPYNILRVAVEGKTLDRWQAEKKPQAEPDRPMILIDNQES
(SEQ ID NO: 50)
CasΦ.6
MDMLDTETNYATETPAQQQDYSPKPPKKAQRAPKGFSKKARPEKKPPKPITLFTQKHFSGVRF
LKRVIRDASKILKLSESRTITFLEQAIERDGSAPPDVTPPVHNTIMAVTRPFEEWPEVILSKALQK
HCYALTKKIKIKTWPKKGPGKKCLAAWSARTKIPLIPGQVQATNGLFDRIGSIYDGVEKKVTN
RNANKKLEYDEAIKEGRNPAVPEYETAYNIDGTLINKPGYNPNLYITQSRTPRLITEADRPLVE
KILWQMVEKKTQSRNQARRARLEKAAHLQGLPVPKFVPEKVDRSQKIEIRIIDPLDKIEPYMPQ
DRMAIKASQDGHVPYWQRPFLSKRRNRRVRAGWGKQVSSIQAWLTGALLVIVRLGNEAFLA
DIRGALRNAQWRKLLKPDATYQSLFNLFTGDPVVNTRINHLTMAYREGVVDIVKSRSFKGRQ
TREHLLTLLGQGKTVAGVSFDLGQKHAAGLLAAHFGLGEDGNPVFTPIQACFLPQRYLDSLT
NYRNRYDALTLDMRRQSLLALTPAQQQEFADAQRDPGGQAKRACCLKLNLNPDEIRWDLVS
GISTMISDLYIERGGDPRDVHQQVETKPKGKRKSEIRILKIRDGKWAYDFRPKIADETRKAQRE
QLWKLQKASSEFERLSRYKINIARAIANWALQWGRELSGCDIVIPVLEDLNVGSKFFDGKGK
WLLGWDNRFTPKKENRWFIKVLHKAVAELAPHKGVPVYEVMPHRTSMTCPACHYCHPTNR
EGDRFECQSCHVVKNTDRDVAPYNILRVAVEGKTLDRWQAEKKPQAEPDRPMILIDNQES
(SEQ ID NO: 51)
CasΦ.7
MSSLPTPLELLKQKHADLFKGLQFSSKDNKMAGKVLKKDGEEAALAFLSERGVSRGELPNFR
PPAKTLVVAQSRPFEEFPIYRVSEAIQLYVYSLSVKELETVPSGSSTKKEHQRFFQDSSVPDFGY
TSVQGLNKIFGLARGIYLGVITRGENQLQKAKSKHEALNKKRRASGEAETEFDPTPYEYMTPE
RKLAKPPGVNHSIMCYVDISVDEFDFRNPDGIVLPSEYAGYCREINTAIEKGTVDRLGHLKGGP
GYIPGHQRKESTTEGPKINFRKGRIRRSYTALYAKRDSRRVRQGKLALPSYRHHMMRLNSNA
ESAILAVIFFGKDWVVFDLRGLLRNVRWRNLFVDGSTPSTLLGMFGDPVIDPKRGVVAFCYKE
QIVPVVSKSITKMVKAPELLNKLYLKSEDPLVLVAIDLGQTNPVGVGVYRVMNASLDYEVVT
RFALESELLREIESYRQRTNAFEAQIRAETFDAMTSEEQEEITRVRAFSASKAKENVCHRFGMP
VDAVDWATMGSNTIHIAKWVMRHGDPSLVEVLEYRKDNEIKLDKNGVPKKVKLTDKRIANL
TSIRLRFSQETSKHYNDTMWELRRKHPVYQKLSKSKADFSRRVVNSIIRRVNHLVPRARIVFIIE
DLKNLGKVFHGSGKRELGWDSYFEPKSENRWFIQVLHKAFSETGKHKGYYIIECWPNWTSCT
CPKCSCCDSENRHGEVFRCLACGYTCNTDFGTAPDNLVKIATTGKGLPGPKKRCKGSSKGKN
PKIARSSETGVSVTESGAPKVKKSSPTQTSQSSSQSAP (SEQ ID NO: 52)
CasΦ.8
MNKIEKEKTPLAKLMNENFAGLRFPFAIIKQAGKKLLKEGELKTIEYMTGKGSIEPLPNFKPPV
KCLIVAKRRDLKYFPICKASCEIQSYVYSLNYKDFMDYFSTPMTSQKQHEEFFKKSGLNIEYQ
NVAGLNLIFNNVKNTYNGVILKVKNRNEKLKKKAIKNNYEFEEIKTFNDDGCLINKPGINNVI
YCFQSISPKILKNITHLPKEYNDYDCSVDRNIIQKYVSRLDIPESQPGHVPEWQRKLPEFNNTNN
PRRRRKWYSNGRNISKGYSVDQVNQAKIEDSLLAQIKIGEDWIILDIRGLLRDLNRRELISYKN
KLTIKDVLGFFSDYPIIDIKKNLVTFCYKEGVIQVVSQKSIGNKKSKQLLEKLIENKPIALVSIDL
GQTNPVSVKISKLNKINNKISIESFTYRFLNEEILKEIEKYRKDYDKLELKLINEA (SEQ ID NO:
53)
CasΦ.9
MDMLDTETNYATETPSQQQDYSPKPPKKDRRAPKGFSKKARPEKKPPKPITLFTQKHFSGVRF
LKRVIRDASKILKLSESRTITFLEQAIERDGSAPPDVTPPVHNTIMAVTRPFEEWPEVILSKALQK
HCYALTKKIKIKTWPKKGPGKKCLAAWSARTKIPLIPGQVQATNGLFDRIGSIYDGVEKKVTN
RNANKKLEYDEAIKEGRNPAVPEYETAYNIDGTLINKPGYNPNLYITQSRTPRLITEADRPLVE
KILWQMVEKKTQSRNQARRARLEKAAHLQGLPVPKFVPEKVDRSQKIEIRIIDPLDKIEPYMPQ
DRMAIKASQDGHVPYWQRPFLSKRRNRRVRAGWGKQVSSIQAWLTGALLVIVRLGNEAFLA
DIRGALRNAQWRKLLKPDATYQSLFNLFTGDPVVNTRTNHLTMAYREGVVDIVKSRSFKGRQ
TREHLLTLLGQGKTVAGVSFDLGQKHAAGLLAAHFGLGEDGNPVFTPIQACFLPQRYLDSLT
NYRNRYDALTLDMRRQSLLALTPAQQQEFADAQRDPGGQAKRACCLKLNLNPDEIRWDLVS
GISTMISDLYIERGGDPRDVHQQVETKPKGKRKSEIRILKIRDGKWAYDFRPKIADETRKAQRE
QLWKLQKASSEFERLSRYKINIARAIANWALQWGRELSGCDIVIPVLEDLNVGSKFFDGKGK
WLLGWDNRFTPKKENRWFIKVLHKAVAELAPHRGVPVYEVMPHRTSMTCPACHYCHPTNRE
GDRFECQSCHVVKNTDRDVAPYNILRVAVEGKTLDRWQAEKKPQAEPDRPMILIDNQES
(SEQ ID NO: 54)
CasΦ.10
MDMLDTETNYATETPSQQQDYSPKPPKKDRRAPKGFSKKARPEKKPPKPITLFTQKHFSGVRF
LKRVIRDASKILKLSESRTITFLEQAIERDGSAPPDVTPPVHNTIMAVTRPFEEWPEVILSKALQK
HCYALTKKIKIKTWPKKGPGKKCLAAWSARTKIPLIPGQVQATNGLFDRIGSIYDGVEKKVTN
RNANKKLEYDEAIKEGRNPAVPEYETAYNIDGTLINKPGYNPNLYITQSRTPRLITEADRPLVE
KILWQMVEKKTQSRNQARRARLEKAAHLQGLPVPKFVPEKVDRSQKIEIRIIDPLDKIEPYMPQ
DRMAIKASQDGHVPYWQRPFLSKRRNRRVRAGWGKQVSSIQAWLTGALLVIVRLGNEAFLA
DIRGALRNAQWRKLLKPDATYQSLFNLFTGDPVVNTRINHLTMAYREGVVNIVKSRSFKGRQ
TREHLLTLLGQGKTVAGVSFDLGQKHAAGLLAAHFGLGEDGNPVFTPIQACFLPQRYLDSLT
NYRNRYDALTLDMRRQSLLALTPAQQQEFADAQRDPGGQAKRACCLKLNLNPDEIRWDLVS
GISTMISDLYIERGGDPRDVHQQVETKPKGKRKSEIRILKIRDGKWAYDFRPKIADETRKAQRE
QLWKLQKASSEFERLSRYKINIARAIANWALQWGRELSGCDIVIPVLEDLNVGSKFFDGKGK
WLLGWDNRFTPKKENRWFIKVLHKAVAELAPHRGVPVYEVMPHRTSMTCPACHYCHPTNRE
GDRFECQSCHVVKNTDRDVAPYNILRVAVEGKTLDRWQAEKKPQAEPDRPMILIDNQES
(SEQ ID NO: 55)
CasΦ.11
MSNKTTPPSPLSLLLRAHFPGLKFESQDYKIAGKKLRDGGPEAVISYLTGKGQAKLKDVKPPA
KAFVIAQSRPFIEWDLVRVSRQIQEKIFGIPATKGRPKQDGLSETAFNEAVASLEVDGKSKLNE
ETRAAFYEVLGLDAPSLHAQAQNALIKSAISIREGVLKKVENRNEKNLSKTKRRKEAGEEATF
VEEKAHDERGYLIHPPGVNQTIPGYQAVVIKSCPSDFIGLPSGCLAKESAEALTDYLPHDRMTI
PKGQPGYVPEWQHPLLNRRKNRRRRDWYSASLNKPKATCSKRSGTPNRKNSRTDQIQSGRFK
GAIPVLMRFQDEWVIIDIRGLLRNARYRKLLKEKSTIPDLLSLFTGDPSIDMRQGVCTFIYKAG
QACSAKMVKTKNAPEILSELTKSGPVVLVSIDLGQTNPIAAKVSRVTQLSDGQLSHETLLRELL
SNDSSDGKEIARYRVASDRLRDKLANLAVERLSPEHKSEILRAKNDTPALCKARVCAALGLNP
EMIAWDKMTPYTEFLATAYLEKGGDRKVATLKPKNRPEMLRRDIKFKGTEGVRIEVSPEAAE
AYREAQWDLQRTSPEYLRLSTWKQELTKRILNQLRHKAAKSSQCEVVVMAFEDLNIKMMHG
NGKWADGGWDAFFIKKRENRWFMQAFHKSLTELGAHKGVPTIEVTPHRTSITCTKCGHCDK
ANRDGERFACQKCGFVAHADLEIATDNIERVALTGKPMPKPESERSGDAKKSVGARKAAFKP
EEDAEAAE (SEQ ID NO: 56)
CasΦ.12
MIKPTVSQFLTPGFKLIRNHSRTAGLKLKNEGEEACKKFVRENEIPKDECPNFQGGPAIANIIAK
SREFTEWEIYQSSLAIQEVIFTLPKDKLPEPILKEEWRAQWLSEHGLDTVPYKEAAGLNLIIKNA
VNTYKGVQVKVDNKNKNNLAKINRKNEIAKLNGEQEISFEEIKAFDDKGYLLQKPSPNKSIYC
YQSVSPKPFITSKYHNVNLPEEYIGYYRKSNEPIVSPYQFDRLRIPIGEPGYVPKWQYTFLSKKE
NKRRKLSKRIKNVSPILGIICIKKDWCVFDMRGLLRTNHWKKYHKPTDSINDLFDYFTGDPVID
TKANVVRFRYKMENGIVNYKPVREKKGKELLENICDQNGSCKLATVDVGQNNPVAIGLFELK
KVNGELTKTLISRHPTPIDFCNKITAYRERYDKLESSIKLDAIKQLTSEQKIEVDNYNNNFTPQN
TKQIVCSKLNINPNDLPWDKMISGTHFISEKAQVSNKSEIYFTSTDKGKTKDVMKSDYKWFQD
YKPKLSKEVRDALSDIEWRLRRESLEFNKLSKSREQDARQLANWISSMCDVIGIENLVKKNNF
FGGSGKREPGWDNFYKPKKENRWWINAIHKALTELSQNKGKRVILLPAMRTSITCPKCKYCD
SKNRNGEKFNCLKCGIELNADIDVATENLATVAITAQSMPKPTCERSGDAKKPVRARKAKAP
EFHDKLAPSYTVVLREAV (SEQ ID NO: 57)
CasΦ.13
MRQPAEKTAFQVFRQEVIGTQKLSGGDAKTAGRLYKQGKMEAAREWLLKGARDDVPPNFQP
PAKCLVVAVSHPFEEWDISKTNHDVQAYIYAQPLQAEGHLNGLSEKWEDTSADQHKLWFEK
TGVPDRGLPVQAINKIAKAAVNRAFGVVRKVENRNEKRRSRDNRIAEHNRENGLTEVVREAP
EVATNADGFLLHPPGIDPSILSYASVSPVPYNSSKHSFVRLPEEYQAYNVEPDAPIPQFVVEDRF
AIPPGQPGYVPEWQRLKCSTNKHRRMRQWSNQDYKPKAGRRAKPLEFQAHLTRERAKGALL
VVMRIKEDWVVFDVRGLLRNVEWRKVLSEEAREKLTLKGLLDLFTGDPVIDTKRGIVTFLYK
AEITKILSKRTVKTKNARDLLLRLTEPGEDGLRREVGLVAVDLGQTHPIAAAIYRIGRTSAGAL
ESTVLHRQGLREDQKEKLKEYRKRHTALDSRLRKEAFETLSVEQQKEIVTVSGSGAQITKDKV
CNYLGVDPSTLPWEKMGSYTHFISDDFLRRGGDPNIVHFDRQPKKGKVSKKSQRIKRSDSQW
VGRMRPRLSQETAKARMEADWAAQNENEEYKRLARSKQELARWCVNTLLQNTRCITQCDEI
VVVIEDLNVKSLHGKGAREPGWDNFFTPKTENRWFIQILHKTFSELPKHRGEHVIEGCPLRTSI
TCPACSYCDKNSRNGEKFVCVACGATFHADFEVATYNLVRLATTGMPMPKSLERQGGGEKA
GGARKARKKAKQVEKIVVQANANVTMNGASLHSP (SEQ ID NO: 58)
CasΦ.17
MYSLEMADLKSEPSLLAKLLRDRFPGKYWLPKYWKLAEKKRLTGGEEAACEYMADKQLDSP
PPNFRPPARCVILAKSRPFEDWPVHRVASKAQSFVIGLSEQGFAALRAAPPSTADARRDWLRS
HGASEDDLMALEAQLLETIMGNAISLHGGVLKKIDNANVKAAKRLSGRNEARLNKGLQELPP
EQEGSAYGADGLLVNPPGLNLNIYCRKSCCPKPVKNTARFVGHYPGYLRDSDSILISGTMDRL
TIIEGMPGHIPAWQREQGLVKPGGRRRRLSGSESNMRQKVDPSTGPRRSTRSGTVNRSNQRTG
RNGDPLLVEIRMKEDWVLLDARGLLRNLRWRESKRGLSCDHEDLSLSGLLALFSGDPVIDPV
RNEVVFLYGEGIIPVRSTKPVGTRQSKKLLERQASMGPLTLISCDLGQTNLIAGRASAISLTHGS
LGVRSSVRIELDPEIIKSFERLRKDADRLETEILTAAKETLSDEQRGEVNSHEKDSPQTAKASLC
RELGLHPPSLPWGQMGPSTTFIADMLISHGRDDDAFLSHGEFPTLEKRKKFDKRFCLESRPLLS
SETRKALNESLWEVKRTSSEYARLSQRKKEMARRAVNFVVEISRRKTGLSNVIVNIEDLNVRIF
HGGGKQAPGWDGFFRPKSENRWFIQAIHKAFSDLAAHHGIPVIESDPQRTSMTCPECGHCDSK
NRNGVRFLCKGCGASMDADFDAACRNLERVALTGKPMPKPSTSCERLLSATTGKVCSDHSLS
HDAIEKAS (SEQ ID NO: 62)
CasΦ.18
MEKEITELTKIRREFPNKKFSSTDMKKAGKLLKAEGPDAVRDFLNSCQEIIGDFKPPVKTNIVSI
SRPFEEWPVSMVGRAIQEYYFSLTKEELESVHPGTSSEDHKSFFNITGLSNYNYTSVQGLNLIF
KNAKAIYDGTLVKANNKNKKLEKKFNEINHKRSLEGLPIITPDFEEPFDENGHLNNPPGINRNI
YGYQGCAAKVFVPSKHKMVSLPKEYEGYNRDPNLSLAGFRNRLEIPEGEPGHVPWFQRMDIP
EGQIGHVNKIQRFNFVHGKNSGKVKFSDKTGRVKRYHHSKYKDATKPYKFLEESKKVSALDS
ILAIITIGDDWVVFDIRGLYRNVFYRELAQKGLTAVQLLDLFTGDPVIDPKKGVVTFSYKEGVV
PVFSQKIVPRFKSRDTLEKLTSQGPVALLSVDLGQNEPVAARVCSLKNINDKITLDNSCRISFLD
DYKKQIKDYRDSLDELEIKIRLEAINSLETNQQVEIRDLDVFSADRAKANTVDMFDIDPNLISW
DSMSDARVSTQISDLYLKNGGDESRVYFEINNKRIKRSDYNISQLVRPKLSDSTRKNLNDSIWK
LKRTSEEYLKLSKRKLELSRAVVNYTIRQSKLLSGINDIVIILEDLDVKKKFNGRGIRDIGWDNF
FSSRKENRWFIPAFHKTFSELSSNRGLCVIEVNPAWTSATCPDCGFCSKENRDGINFTCRKCGV
SYHADIDVATLNIARVAVLGKPMSGPADRERLGDTKKPRVARSRKTMKRKDISNSTVEAMVT
A (SEQ ID NO: 63)
CasΦ.19
MLVRTSTLVQDNKNSRSASRAFLKKPKMPKNKHIKEPTELAKLIRELFPGQRFTRAINTQAGKI
LKHKGRDEVVEFLKNKGIDKEQFMDFRPPTKARIVATSGAIEEFSYLRVSMAIQECCFGKYKF
PKEKVNGKLVLETVGLTKEELDDFLPKKYYENKKSRDRFFLKTGICDYGYTYAQGLNEIFRNT
RAIYEGVFTKVNNRNEKRREKKDKYNEERRSKGLSEEPYDEDESATDESGHLINPPGVNLNIW
TCEGFCKGPYVTKLSGTPGYEVILPKVFDGYNRDPNEIISCGITDRFAIPEGEPGHIPWHQRLEIP
EGQPGYVPGHQRFADTGQNNSGKANPNKKGRMRKYYGHGTKYTQPGEYQEVFRKGHREGN
KRRYWEEDFRSEAHDCILYVIHIGDDWVVCDLRGPLRDAYRRGLVPKEGITTQELCNLFSGDP
VIDPKHGVVTFCYKNGLVRAQKTISAGKKSRELLGALTSQGPIALIGVDLGQTEPVGARAFIVN
QARGSLSLPTLKGSFLLTAENSSSWNVFKGEIKAYREAIDDLAIRLKKEAVATLSVEQQTEIES
YEAFSAEDAKQLACEKFGVDSSFILWEDMTPYHTGPATYYFAKQFLKKNGGNKSLIEYIPYQK
KKSKKTPKAVLRSDYNIACCVRPKLLPETRKALNEAIRIVQKNSDEYQRLSKRKLEFCRRVVN
YLVRKAKKLTGLERVIIAIEDLKSLEKFFTGSGKRDNGWSNFFRPKKENRWFIPAFHKAFSELA
PNRGFYVIECNPARTSITDPDCGYCDGDNRDGIKFECKKCGAKHHTDLDVAPLNIAIVAVTGR
PMPKTVSNKSKRERSGGEKSVGASRKRNHRKSKANQEMLDATSSAAE (SEQ ID NO: 64)
CasΦ.20
MPKIKKPTEISLLRKEVFPDLHFAKDRMRAASLVLKNEGREAAIEYLRVNHEDKPPNFMPPAK
TPYVALSRPLEQWPIAQASIAIQKYIFGLTKDEFSATKKLLYGDKSTPNTESRKRWFEVTGVPN
FGYMSAQGLNAIFSGALARYEGVVQKVENRNKKRFEKLSEKNQLLIEEGQPVKDYVPDTAYH
TPETLQKLAENNHVRVEDLGDMIDRLVHPPGIHRSIYGYQQVPPFAYDPDNPKGIILPKAYAG
YTRKPHDIIEAMPNRLNIPEGQAGYIPEHQRDKLKKGGRVKRLRTTRVRVDATETVRAKAEAL
NAEKARLRGKEAILAVFQIEEDWALIDMRGLLRNVYMRKLIAAGELTPTTLLGYFTETLTLDP
RRTEATFCYHLRSEGALHAEYVRHGKNTRELLLDLTKDNEKIALVTIDLGQRNPLAAAIFRVG
RDASGDLTENSLEPVSRMLLPQAYLDQIKAYRDAYDSFRQNIWDTALASLTPEQQRQILAYEA
YTPDDSKENVLRLLLGGNVMPDDLPWEDMTKNTHYISDRYLADGGDPSKVWFVPGPRKRKK
NAPPLKKPPKPRELVKRSDHNISHLSEFRPQLLKETRDAFEKAKIDTERGHVGYQKLSTRKDQ
LCKEILNWLEAEAVRLTRCKTMVLGLEDLNGPFFNQGKGKVRGWVSFFRQKQENRWIVNGF
RKNALARAHDKGKYILELWPSWTSQTCPKCKHVHADNRHGDDFVCLQCGARLHADAEVAT
WNLAVVAIQGHSLPGPVREKSNDRKKSGSARKSKKANESGKVVGAWAAQATPKRATSKKET
GTARNPVYNPLETQASCPAP (SEQ ID NO: 65)
CasΦ.21
MTPSPQIARLVETPLAAALKAHHPGKKFRSDYLKKAGKILKDQGVEAAMAHLDGKDQAEPP
NFKPPAKCRIVARSREFSEWPIVKASVEIQKYIYGLTLEERKACDPGKSSASHKAWFAKTGVN
TFGYSSVQGFNLIFGHTLGRYDGVLVKTENLNKKRAEKNERFRAKALAEGRAEPVCPPLVTA
TNDTGQDVTLEDGRVVRPGQLLQPPGINPNIYAYQQVSPKAYVPGIIELPEEFQGYSRDPNAVI
LPLVPRDRLSIPKGQPGYVPEPHREGLTGRKDRRMRRYYETERGTKLKRPPLTAKGRADKAN
EALLVVVRIDSDWVVMDVRGLLRNARWRRLVSKEGITLNGLLDLFTGDPVLNPKDCSVSRDT
GDPVNDPRHGVVTFCYKLGVVDVCSKDRPIKGFRTKEVLERLTSSGTVGMVSIDLGQTNPVA
AAVSRVTKGLQAETLETFTLPDDLLGKVRAYRAKTDRMEEGFRRNALRKLTAEQQAEITRYN
DATEQQAKALVCSTYGIGPEEVPWERMTSNTTYISDHILDHGGDPDTVFFMATKRGQNKPTL
HKRKDKAWGQKFRPAISVETRLARQAAEWELRRASLEFQKLSVWKTELCRQAVNYVMERT
KKRTQCDVIIPVIEDLPVPLFHGSGKRDPGWANFFVHKRENRWFIDGLHKAFSELGKHRGIYV
FEVCPQRTSITCPKCGHCDPDNRDGEKFVCLSCQATLNADLDVATTNLVRVALTGKVMPRSE
RSGDAQTPGPARKARTGKIKGSKPTSAPQGATQTDAKAHLSQTGV (SEQ ID NO: 66)
CasΦ.22
MTPSPQIARLVETPLAAALKAHHPGKKFRSDYLKKAGKILKDQGVEAAMAHLDGKDQAEPP
NFKPPAKCRIVARSREFSEWPIVKASVEIQKYIYGLTLEERKACDPGKSSASHKAWFAKTGVN
TFGYSSVQGFNLIFGHTLGRYDGVLVKTENLNKKRAEKNERFRAKALAEGRAEPVCPPLVTA
TNDTGQDVTLEDGRVVRPGQLLQPPGINPNIYAYQQVSPKAYVPGIIELPEEFQGYSRDPNAVI
LPLVPRDRLSIPKGQPGYVPEPHREGLTGRKDRRMRRYYETERGTKLKRPPLTAKGRADKAN
EALLVVVRIDSDWVVMDVRGLLRNARWRRLVSKEGITLNGLLDLFTGDPVLNPKDCSVSRDT
GDPVNDPRHGVVTFCYKLGVVDVCSKDRPIKGFRTKEVLERLTSSGTVGMVSIDLGQTNPVA
AAVSRVTKGLQAETLETFTLPDDLLGKVRAYRAKTDRMEEGFRRNALRKLTAEQQAEITRYN
DATEQQAKALVCSTYGIGPEEVPWERMTSNTTYISDHILDHGGDPDTVFFMATKRGQNKPTL
HKRKDKAWGQKFRPAISVETRLARQAAEWELRRASLEFQKLSVWKTELCRQAVNYVMERT
KKRTQCDVIIPVIEDLPVPLFHGSGKRDPGWANFFVHKRENRWFIDGLHKAFSELGKHRGIYV
FEVCPQRTSITCPKCGHCDPDNRDGEKFVCLSCQATLHADLDVATTNLVRVALTGKVMPRSE
RSGDAQTPGPARKARTGKIKGSKPTSAPQGATQTDAKAHLSQTGV (SEQ ID NO: 67)
CasΦ.23
MKTEKPKTALTLLREEVFPGKKYRLDVLKEAGKKLSTKGREATIEFLTGKDEERPQNFQPPAK
TSIVAQSRPFDQWPIVQVSLAVQKYIYGLTQSEFEANKKALYGETGKAISTESRRAWFEATGV
DNFGFTAAQGINPIFSQAVARYEGVIKKVENRNEKKLKKLTKKNLLRLESGEEIEDFEPEATFN
EEGRLLQPPGANPNIYCYQQISPRIYDPSDPKGVILPQIYAGYDRKPEDIISAGVPNRLAIPEGQP
GYIPEHQRAGLKTQGRIRCRASVEAKARAAILAVVHLGEDWVVLDLRGLLRNVYWRKLASP
GTLTLKGLLDFFTGGPVLDARRGIATFSYTLKSAAAVHAENTYKGKGTREVLLKLTENNSVA
LVTVDLGQRNPLAAMIARVSRTSQGDLTYPESVEPLTRLFLPDPFLEEVRKYRSSYDALRLSIR
EAAIASLTPEQQAEIRYIEKFSAGDAKKNVAEVFGIDPTQLPWDAMTPRTTYISDLFLRMGGDR
SRVFFEVPPKKAKKAPKKPPKKPAGPRIVKRTDGMIARLREIRPRLSAETNKAFQEARWEGER
SNVAFQKLSVRRKQFARTVVNHLVQTAQKMSRCDTVVLGIEDLNVPFFHGRGKYQPGWEGF
FRQKKENRWLINDMHKALSERGPHRGGYVLELTPFWTSLRCPKCGHTDSANRDGDDFVCVK
CGAKLHSDLEVATANLALVAITGQSIPRPPREQSSGKKSTGTARMKKTSGETQGKGSKACVSE
ALNKIEQGTARDPVYNPLNSQVSCPAP (SEQ ID NO: 68)
CasΦ.24
VYNPDMKKPNNIRRIREEHFEGLCFGKDVLTKAGKIYEKDGEEAAIDFLMGKDEEDPPNFKPP
AKTTIVAQSRPFDQWPIYQVSQAVQERVFAYTEEEFNASKEALFSGDISSKSRDFWFKTNNISD
QGIGAQGLNTILSHAFSRYSGVIKKVENRNKKRLKKLSKKNQLKIEEGLEILEFKPDSAFNENG
LLAQPPGINPNIYGYQAVTPFVFDPDNPGDVILPKQYEGYSRKPDDIIEKGPSRLDIPKGQPGYV
PEHQRKNLKKKGRVRLYRRTPPKTKALASILAVLQIGKDWVLFDMRGLLRSVYMREAATPG
QISAKDLLDTFTGCPVLNTRTGEFTFCYKLRSEGALHARKIYTKGETRTLLTSLTSENNTIALVT
VDLGQRNPAAIMISRLSRKEELSEKDIQPVSRRLLPDRYLNELKRYRDAYDAFRQEVRDEAFT
SLCPEHQEQVQQYEALTPEKAKNLVLKHFFGTHDPDLPWDDMTSNTHYIANLYLERGGDPSK
VFFTRPLKKDSKSKKPRKPTKRTDASISRLPEIRPKMPEDARKAFEKAKWEIYTGHEKFPKLAK
RVNQLCREIANWIEKEAKRLTLCDTVVVGIEDLSLPPKRGKGKFQETWQGFFRQKFENRWVI
DTLKKAIQNRAHDKGKYVLGLAPYWTSQRCPACGFIHKSNRNGDHFKCLKCEALFHADSEV
ATWNLALVAVLGKGITNPDSKKPSGQKKTGTTRKKQIKGKNKGKETVNVPPTTQEVEDIIAFF
EKDDETVRNPVYKPTGT (SEQ ID NO: 69)
CasΦ.25
MKKPNNIRRIREEHFEGLCFGKDVLTKAGKIYEKDGEEAAIDFLMGKDEEDPPNFKPPAKTTIV
AQSRPFDQWPIYQVSQAVQERVFAYTEEEFNASKEALFSGDISSKSRDFWFKTNNISDQGIGAQ
GLNTILSHAFSRYSGVIKKVENRNKKRLKKLSKKNQLKIEEGLEILEFKPDSAFNENGLLAQPP
GINPNIYGYQAVTPFVFDPDNPGDVILPKQYEGYSRKPDDIIEKGPSRLDIPKGQPGYVPEHQR
KNLKKKGRVRLYRRTPPKTKALASILAVLQIGKDWVLFDMRGLLRSVYMREAATPGQISAKD
LLDTFTGCPVLNTRTGEFTFCYKLRSEGALHARKIYTKGETRTLLTSLTSENNTIALVTVDLGQ
RNPAAIMISRLSRKEELSEKDIQPVSRRLLPDRYLNELKRYRDAYDAFRQEVRDEAFTSLCPEH
QEQVQQYEALTPEKAKNLVLKHFFGTHDPDLPWDDMTSNTHYIANLYLERGGDPSKVFFTRP
LKKDSKSKKPRKPTKRTDASISRLPEIRPKMPEDARKAFEKAKWEIYTGHEKFPKLAKRVNQL
CREIANWIEKEAKRLTLCDTVVVGIEDLSLPPKRGKGKFQETWQGFFRQKFENRWVIDTLKKA
IQNRAHDKGKYVLGLAPYWTSQRCPACGFIHKSNRNGDHFKCLKCEALFHADSEVATWNLA
LVAVLGKGITNPDSKKPSGQKKTGTTRKKQIKGKNKGKETVNVPPTTQEVEDIIAFFEKDDET
VRNPVYKPTGT (SEQ ID NO: 70)
CasΦ.26
VIKTHFPAGRFRKDHQKTAGKKLKHEGEEACVEYLRNKVSDYPPNFKPPAKGTIVAQSRPFSE
WPIVRASEAIQKYVYGLTVAELDVFSPGTSKPSHAEWFAKTGVENYGYRQVQGLNTIFQNTV
NRFKGVLKKVENRNKKSLKRQEGANRRRVEEGLPEVPVTVESATDDEGRLLQPPGVNPSIYG
YQGVAPRVCTDLQGFSGMSVDFAGYRRDPDAVLVESLPEGRLSIPKGERGYVPEWQRDPERN
KFPLREGSRRQRKWYSNACHKPKPGRTSKYDPEALKKASAKDALLVSISIGEDWAIIDVRGLL
RDARRRGFTPEEGLSLNSLLGLFTEYPVFDVQRGLITFTYKLGQVDVHSRKTVPTFRSRALLES
LVAKEEIALVSVDLGQTNPASMKVSRVRAQEGALVAEPVHRMFLSDVLLGELSSYRKRMDAF
EDAIRAQAFETMTPEQQAEITRVCDVSVEVARRRVCEKYSISPQDVPWGEMTGHSTFIVDAVL
RKGGDESLVYFKNKEGETLKFRDLRISRMEGVRPRLTKDTRDALNKAVLDLKRAHPTFAKLA
KQKLELARRCVNFIEREAKRYTQCERVVFVIEDLNVGFFHGKGKRDRGWDAFFTAKKENRW
VIQALHKAFSDLGLHRGSYVIEVTPQRTSMTCPRCGHCDKGNRNGEKFVCLQCGATLHADLE
VATDNIERVALTGKAMPKPPVRERSGDVQKAGTARKARKPLKPKQKTEPSVQEGSSDDGVD
KSPGDASRNPVYNPSDTLSI (SEQ ID NO: 71)
CasΦ.27
MAKAKTLAALLRELLPGQHLAPHHRWVANKLLMTSGDAAAFVIGKSVSDPVRGSFRKDVIT
KAGRIFKKDGPDAAAAFLDGKWEDRPPNFQPPAKAAIVAISRSFDEWPIVKVSCAIQQYLYAL
PVQEFESSVPEARAQAHAAWFQDTGVDDCNFKSTQGLNAIFNHGKRTYEGVLKKAQNRNDK
KNLRLERINAKRAEAGQAPLVAGPDESPTDDAGCLLHPPGINANIYCYQQVSPRPYEQSCGIQL
PPEYAGYNRLSNVAIPPMPNRLDIPQGQPGYVPEHHRHGIKKFGRVRKRYGVVPGRNRDADG
KRTRQVLTEAGAAAKARDSVLAVIRIGDDWTVVDLRGLLRNAQWRKLVPDGGITVQGLLDL
FTGDPVIDPRRGVVTFIYKADSVGIHSEKVCRGKQSKNLLERLCAMPEKSSTRLDCARQAVAL
VSVDLGQRNPVAARFSRVSLAEGQLQAQLVSAQFLDDAMVAMIRSYREEYDRFESLVREQA
KAALSPEQLSEIVRHEADSAESVKSCVCAKFGIDPAGLSWDKMTSGTWRIADHVQAAGGDVE
WFFFKTCGKGKEIKTVRRSDFNVAKQFRLRLSPETRKDWNDAIWELKRGNPAYVSFSKRKSE
FARRVVNDLVHRARRAVRCDEVVFAIEDLNISFFHGKGQRQMGWDAFFEVKQENRWFIQAL
HKAFVERATHKGGYVLEVAPARTSTTCPECRHCDPESRRGEQFCCIKCRHTCHADLEVATFNI
EQVALTGVSLPKRLSSTLL (SEQ ID NO: 72)
CasΦ.28
MSKEKTPPSAYAILKAKHFPDLDFEKKHKMMAGRMFKNGASEQEVVQYLQGKGSESLMDV
KPPAKSPILAQSRPFDEWEMVRTSRLIQETIFGIPKRGSIPKRDGLSETQFNELVASLEVGGKPM
LNKQTRAIFYGLLGIKPPTFHAMAQNILIDLAINIRKGVLKKVDNLNEKNRKKVKRIRDAGEQ
DVMVPAEVTAHDDRGYLNHPPGVNPTIPGYQGVVIPFPEGFEGLPSGMTPVDWSHVLVDYLP
HDRLSIPKGSPGYIPEWQRPLLNRHKGRRHRSWYANSLNKPRKSRTEEAKDRQNAGKRTALIE
AERLKGVLPVLMRFKEDWLIIDARGLLRNARYRGVLPEGSTLGNLIDLFSDSPRVDTRRGICTF
LYRKGRAYSTKPVKRKESKETLLKLTEKSTIALVSIDLGQTNPLTAKLSKVRQVDGCLVAEPV
LRKLIDNASEDGKEIARYRVAHDLLRARILEDAIDLLGIYKDEVVRARSDTPDLCKERVCRFLG
LDSQAIDWDRMTPYTDFIAQAFVAKGGDPKVVTIKPNGKPKMFRKDRSIKNMKGIRLDISKEA
SSAYREAQWAIQRESPDFQRLAVWQSQLTKRIVNQLVAWAKKCTQCDTVVLAFEDLNIGMM
HGSGKWANGGWNALFLHKQENRWFMQAFHKALTELSAHKGIPTIEVLPHRTSITCTQCGHCH
PGNRDGERFKCLKCEFLANTDLEIATDNIERVALTGLPMPKGERSSAKRKPGGTRKTKKSKHS
GNSPLAAE (SEQ ID NO: 73)
CasΦ.29
MEKAGPTSPLSVLIHKNFEGCRFQIDHLKIAGRKLAREGEAAAIEYLLDKKCEGLPPNFQPPAK
GNVIAQSRPFTEWAPYRASVAIQKYIYSLSVDERKVCDPGSSSDSHEKWFKQTGVQNYGYTH
VQGLNLIFKHALARYDGVLKKVDNRNEKNRKKAERVNSFRREEGLPEEVFEEEKATDETGHL
LQPPGVNHSIYCYQSVRPKPFNPRKPGGISLPEAYSGYSLKPQDELPIGSLDRLSIPPGQPGYVPE
WQRSQLTTQKHRRKRSWYSAQKWKPRTGRTSTFDPDRLNCARAQGAILAVVRIHEDWVVFD
VRGLLRNALWRELAGKGLTVRDLLDFFTGDPVVDTKRGVVTFTYKLGKVDVHSLRTVRGKR
SKKVLEDLTLSSDVGLVTIDLGQTNVLAADYSKVTRSENGELLAVPLSKSFLPKHLLHEVTAY
RTSYDQMEEGFRRKALLTLTEDQQVEVTLVRDFSVESSKTKLLQLGVDVTSLPWEKMSSNTT
YISDQLLQQGADPASLFFDGERDGKPCRHKKKDRTWAYLVRPKVSPETRKALNEALWALKN
TSPEFESLSKRKIQFSRRCMNYLLNEAKRISGCGQVVFVIEDLNVRVHHGRGKRAIGWDNFFK
PKRENRWFMQALHKAASELAIHRGMHIIEACPARSSITCPKCGHCDPENRCSSDREKFLCVKC
GAAFHADLEVATFNLRKVALTGTALPKSIDHSRDGLIPKGARNRKLKEPQANDEKACA (SEQ
ID NO: 74)
CasΦ.30
MKEQSPLSSVLKSNFPGKKFLSADIRVAGRKLAQLGEAAAVEYLSPRQRDSVPNFRPPAFCTV
VAKSRPFEEWPIYKASVLLQEQIYGMTGQEFEERCGSIPTSLSGLRQWASSVGLGAAMEGLHV
QGMNLMVKNAINRYKGVLVKVENRNKKLVEANEAKNSSREERGLPPLRPPELGSAFGPDGR
LVNPPGIDKSIRLYQGVSPVPVVKTTGRPTVHRLDIPAGEKGHVPLWQREAGLVKEGPRRRRM
WYSNSNLKRSRKDRSAEASEARKADSVVVRVSVKEDWVDIDVRGLLRNVAWRGIERAGEST
EDLLSLFSGDPVVDPSRDSVVFLYKEGVVDVLSKKVVGAGKSRKQLEKMVSEGPVALVSCDL
GQTNYVAARVSVLDESLSPVRSFRVDPREFPSADGSQGVVGSLDRIRADSDRLEAKLLSEAEA
SLPEPVRAEIEFLRSERPSAVAGRLCLKLGIDPRSIPWEKMGSTTSFISEALSAKGSPLALHDGAP
IKDSRFAHAARGRLSPESRKALNEALWERKSSSREYGVISRRKSEASRRMANAVLSESRRLTG
LAVVAVNLEDLNMVSKFFHGRGKRAPGWAGFFTPKMENRWFIRSIHKAMCDLSKHRGITVIE
SRPERTSISCPECGHCDPENRSGERFSCKSCGVSLHADFEVATRNLERVALTGKPMPRRENLHS
PEGATASRKTRKKPREATASTFLDLRSVLSSAENEGSGPAARAG (SEQ ID NO: 75)
CasΦ.31
MLPPSNKIGKSMSLKEFINKRNFKSSIIKQAGKILKKEGEEAVKKYLDDNYVEGYKKRDFPITA
KCNIVASNRKIEDFDISKFSSFIQNYVFNLNKDNFEEFSKIKYNRKSFDELYKKIANEIGLEKPN
YENIQGEIAVIRNAINIYNGVLKKVENRNKKIQEKNQSKDPPKLLSAFDDNGFLAERPGINETIY
GYQSVRLRHLDVEKDKDIIVQLPDIYQKYNKKSTDKISVKKRLNKYNVDEYGKLISKRRKERI
NKDDAILCVSNFGDDWIIFDARGLLRQTYRYKLKKKGLCIKDLLNLFTGDPIINPTKTDLKEAL
SLSFKDGIINNRTLKVKNYKKCPELISELIRDKGKVAMISIDLGQTNPISYRLSKFTANNVAYIE
NGVISEDDIVKMKKWREKSDKLENLIKEEAIASLSDDEQREVRLYENDIADNTKKKILEKFNIR
EEDLDFSKMSNNTYFIRDCLKNKNIDESEFTFEKNGKKLDPTDACFAREYKNKLSELTRKKINE
KIWEIKKNSKEYHKISIYKKETIRYIVNKLIKQSKEKSECDDIIVNIEKLQIGGNFFGGRGKRDPG
WNNFFLPKEENRWFINACHKAFSELAPHKGIIVIESDPAYTSQTCPKCENCDKENRNGEKFKC
KKCNYEANADIDVATENLEKIAKNGRRLIKNFDQLGERLPGAEMPGGARKRKPSKSLPKNGR
GAGVGSEPELINQSPSQVIA (SEQ ID NO: 76)
CasΦ.32
VPDKKETPLVALCKKSFPGLRFKKHDSRQAGRILKSKGEGAAVAFLEGKGGTTQPNFKPPVK
CNIVAMSRPLEEWPIYKASVVIQKYVYAQSYEEFKATDPGKSEAGLRAWLKATRVDTDGYFN
VQGLNLIFQNARATYEGVLKKVENRNSKKVAKIEQRNEHRAERGLPLLTLDEPETALDETGH
LRHRPGINCSVFGYQHMKLKPYVPGSIPGVTGYSRDPSTPIAACGVDRLEIPEGQPGYVPPWDR
ENLSVKKHRRKRASWARSRGGAIDDNMLLAVVRVADDWALLDLRGLLRNTQYRKLLDRSV
PVTIESLLNLVTNDPTLSVVKKPGKPVRYTATLIYKQGVVPVVKAKVVKGSYVSKMLDDTTE
TFSLVGVDLGVNNLIAANALRIRPGKCVERLQAFTLPEQTVEDFFRFRKAYDKHQENLRLAAV
RSLTAEQQAEVLALDTFGPEQAKMQVCGHLGLSVDEVPWDKVNSRSSILSDLAKERGVDDTL
YMFPFFKGKGKKRKTEIRKRWDVNWAQHFRPQLTSETRKALNEAKWEAERNSSKYHQLSIR
KKELSRHCVNYVIRTAEKRAQCGKVIVAVEDLHHSFRRGGKGSRKSGWGGFFAAKQEGRWL
MDALFGAFCDLAVHRGYRVIKVDPYNTSRTCPECGHCDKANRDRVNREAFICVCCGYRGNA
DIDVAAYNIAMVAITGVSLRKAARASVASTPLESLAAE (SEQ ID NO: 77)
CasΦ.33
MSKTKELNDYQEALARRLPGVRHQKSVRRAARLVYDRQGEDAMVAFLDGKEVDEPYTLQPP
AKCHILAVSRPIEEWPIARVTMAVQEHVYALPVHEVEKSRPETTEGSRSAWFKNSGVSNHGVT
HAQTLNAILKNAYNVYNGVIKKVENRNAKKRDSLAAKNKSRERKGLPHFKADPPELATDEQ
GYLLQPPSPNSSVYLVQQHLRTPQIDLPSGYTGPVVDPRSPIPSLIPIDRLAIPPGQPGYVPLHDR
EKLTSNKHRRMKLPKSLRAQGALPVCFRVFDDWAVVDGRGLLRHAQYRRLAPKNVSIAELL
ELYTGDPVIDIKRNLMTFRFAEAVVEVTARKIVEKYHNKYLLKLTEPKGKPVREIGLVSIDLNV
QRLIALAIYRVHQTGESQLALSPCLHREILPAKGLGDFDKYKSKFNQLTEEILTAAVQTLTSAQ
QEEYQRYVEESSHEAKADLCLKYSITPHELAWDKMTSSTQYISRWLRDHGWNASDFTQITKG
RKKVERLWSDSRWAQELKPKLSNETRRKLEDAKHDLQRANPEWQRLAKRKQEYSRHLANT
VLSMAREYTACETVVIAIENLPMKGGFVDGNGSRESGWDNFFTHKKENRWMIKDIHKALSDL
APNRGVHVLEVNPQYTSQTCPECGHRDKANRDPIQRERFCCTHCGAQRHADLEVATHNIAM
VATTGKSLTGKSLAPQRLQEAAE (SEQ ID NO: 78)
CasΦ.34
MTPSFGYQMIIVTPIHHASGAWATLRLLFLNPKTSGVMLGMTKTKSAFALMREEVFPGLLFKS
ADLKMAGRKFAKEGREAAIEYLRGKDEERPANFKPPAKGDIIAQSRPFDQWPIVQVSQAIQKY
IFGLTKAEFDATKTLLYGEGNHPTTESRRRWFEATGVPDFGFTSAQGLNAIFSSALARYEGVIQ
KVENRNEKRLKKLSEKNQRLVEEGHAVEAYVPETAFHTLESLKALSEKSLVPLDDLMDKIDR
LAQPPGINPCLYGYQQVAPYIYDPENPRGVVLPDLYLGYCRKPDDPITACPNRLDIPKGQPGYI
PEHQRGQLKKHGRVRRFRYTNPQAKARAKAQTAILAVLRIDEDWVVMDLRGLLRNVYFREV
AAPGELTARTLLDTFTGCPVLNLRSNVVTFCYDIESKGALHAEYVRKGWATRNKLLDLTKDG
QSVALLSVDLGQRHPVAVMISRLKRDDKGDLSEKSIQVVSRTFADQYVDKLKRYRVQYDAL
RKEIYDAALVSLPPEQQAEIRAYEAFAPGDAKANVLSVMFQGEVSPDELPWDKMNTNTHYIS
DLYLRRGGDPSRVFFVPQPSTPKKNAKKPPAPRKPVKRTDENVSHMPEFRPHLSNETREAFQK
AKWTMERGNVRYAQLSRFLNQIVREANNWLVSEAKKLTQCQTVVWAIEDLHVPFFHGKGK
YHETWDGFFRQKKEDRWFVNVFHKAISERAPNKGEYVMEVAPYRTSQRCPVCGFVDADNRH
GDHFKCLRCGVELHADLEVATWNIALVAVQGHGIAGPPREQSCGGETAGTARKGKNIKKNK
GLADAVTVEAQDSEGGSKKDAGTARNPVYIPSESQVNCPAP (SEQ ID NO: 79)
CasΦ.35
MKPKTPKPPKTPVAALIDKHFPGKRFRASYLKSVGKKLKNQGEDVAVRFLTGKDEERPPNFQ
PPAKSNIVAQSRPIEEWPIHKVSVAVQEYVYGLTVAEKEACSDAGESSSSHAAWFAKTGVENF
GYTSVQGLNKIFPPTFNRFDGVIKKVENRNEKKRQKATRINEAKRNKGQSEDPPEAEVKATDD
AGYLLQPPGINHSVYGYQSITLCPYTAEKFPTIKLPEEYAGYHSNPDAPIPAGVPDRLAIPEGQP
GHVPEEHRAGLSTKKHRRVRQWYAMANWKPKPKRTSKPDYDRLAKARAQGALLIVIRIDED
WVVVDARGLLRNVRWRSLGKREITPNELLDLFTGDPVLDLKRGVVTFTYAEGVVNVCSRSTT
KGKQTKVLLDAMTAPRDGKKRQIGMVAVDLGQTNPIAAEYSRVGKNAAGTLEATPLSRSTLP
DELLREIALYRKAHDRLEAQLREEAVLKLTAEQQAENARYVETSEEGAKLALANLGVDTSTL
PWDAMTGWSTCISDHLINHGGDTSAVFFQTIRKGTKKLETIKRKDSSWADIVRPRLTKETREA
LNDFLWELKRSHEGYEKLSKRLEELARRAVNHVVQEVKWLTQCQDIVIVIEDLNVRNFHGGG
KRGGGWSNFFTVKKENRWFMQALHKAFSDLAAHRGIPVLEVYPARTSITCLGCGHCDPENRD
GEAFVCQQCGATFHADLEVATRNIARVALTGEAMPKAPAREQPGGAKKRGTSRRRKLTEVA
VKSAEPTIHQAKNQQLNGTSRDPVYKGSELPAL (SEQ ID NO: 80)
CasΦ.36
MSDNKMKKLSKEEKPLTPLQILIRKYIDKSQYPSGFKTTIIKQAGVRIKSVKSEQDEINLANWII
SKYDPTYIKRDFNPSAKCQIIATSRSVADFDIVKMSNKVQEIFFASSHLDKNVFDIGKSKSDHDS
WFERNNVDRGIYTYSNVQGMNLIFSNTKNTYLGVAVKAQNKFSSKMKRIQDINNFRITNHQS
PLPIPDEIKIYDDAGFLLNPPGVNPNIFGYQSCLLKPLENKEIISKTSFPEYSRLPADMIEVNYKIS
NRLKFSNDQKGFIQFKDKLNLFKINSQELFSKRRRLSGQPILLVASFGDDWVVLDGRGLLRQV
YYRGIAKPGSITISELLGFFTGDPIVDPIRGVVSLGFKPGVLSQETLKTTSARIFAEKLPNLVLNN
NVGLMSIDLGQTNPVSYRLSEITSNMSVEHICSDFLSQDQISSIEKAKTSLDNLEEEIAIKAVDHL
SDEDKINFANFSKLNLPEDTRQSLFEKYPELIGSKLDFGSMGSGTSYIADELIKFENKDAFYPSG
KKKFDLSFSRDLRKKLSDETRKSYNDALFLEKRTNDKYLKNAKRRKQIVRTVANSLVSKIEEL
GLTPVINIENLAMSGGFFDGRGKREKGWDNFFKVKKENRWVMKDFHKAFSELSPHHGVIVIE
SPPYCTSVTCTKCNFCDKKNRNGHKFTCQRCGLDANADLDIATENLEKVAISGKRMPGSERSS
DERKVAVARKAKSPKGKAIKGVKCTITDEPALLSANSQDCSQSTS (SEQ ID NO: 81)
CasΦ.37
MALSLAEVRERHFKGLRFRSSYLKRAGKILKKEGEAACVAYLTGKDEESPPNFKPPAKCDVV
AQSRPFEEWPIVQASVAVQSYVYGLTKEAFEAFNPGTTKQSHEACLAATGIDTCGYSNVQGL
NLIFRQAKNRYEGVITKVENRNKKAKKKLTRKNEWRQKNGHSELPEAPEELTFNDEGRLLQP
PGINPSLYTYQQISPTPWSPKDSSILPPQYAGYERDPNAPIPFGVAKDRLTIASGCPGYIPEWMR
TAGEKTNPRTQKKFMHPGLSTRKNKRMRLPRSVRSAPLGALLVTIHLGEDWLVLDVRGLLRN
ARWRGVAPKDISTQGLLNLFTGDPVIDTRRGVVTFTYKPETVGIHSRTWLYKGKQTKEVLEK
LTQDQTVALVAIDLGQTNPVSAAASRVSRSGENLSIETVDRFFLPDELIKELRLYRMAHDRLEE
RIREESTLALTEAQQAEVRALEHVVRDDAKNKVCAAFNLDAASLPWDQMTSNTTYLSEAILA
QGVSRDQVFFTPNPKKGSKEPVEVMRKDRAWVYAFKAKLSEETRKAKNEALWALKRASPD
YARLSKRREELCRRSVNMVINRAKKRTQCQVVIPVLEDLNIGFFHGSGKRLPGWDNFFVAKK
ENRWLMNGLHKSFSDLAVHRGFYVFEVMPHRTSITCPACGHCDSENRDGEAFVCLSCKRTYH
ADLDVATHNLTQVAGTGLPMPEREHPGGTKKPGGSRKPESPQTHAPILHRTDYSESADRLGS
(SEQ ID NO: 82)
CasΦ.38
MIKEQSELSKLIEKYYPGKKFYSNDLKQAGKHLKKSEHLTAKESEELTVEFLKSCKEKLYDFR
PPAKALIISTSRPFEEWPIYKASESIQKYIYSLTKEELEKYNISTDKTSQENFFKESLIDNYGFANV
SGLNLIFQHTKAIYDGVLKKVNNRNNKILKKYKRKIEEGIEIDSPELEKAIDESGHFINPPGINKN
IYCYQQVSPTIFNSFKETKIICPFNYKRNPNDIIQKGVIDRLAIPFGEPGYIPDHQRDKVNKHKKR
IRKYYKNNENKNKDAILAKINIGEDWVLFDLRGLLRNAYWRKLIPKQGITPQQLLDMFSGDPV
IDPIKNNITFIYKESIIPIHSESIIKTKKSKELLEKLTKDEQIALVSIDLGQTNPVAARFSRLSSDLKP
EHVSSSFLPDELKNEICRYREKSDLLEIEIKNKAIKMLSQEQQDEIKLVNDISSEELKNSVCKKY
NIDNSKIPWDKMNGFTTFIADEFINNGGDKSLVYFTAKDKKSKKEKLVKLSDKKIANSFKPKIS
KETREILNKITWDEKISSNEYKKLSKRKLEFARRATNYLINQAKKATRLNNVVLVVEDLNSKF
FHGSGKREDGWDNFFIPKKENRWFIQALHKSLTDVSIHRGINVIEVRPERTSITCPKCGCCDKE
NRKGEDFKCIKCDSVYHADLEVATFNIEKVAITGESMPKPDCERLGGEESIG (SEQ ID NO: 83)
CasΦ.39
VAFLDGKEVDEPYTLQPPAKCHILAVSRPIEEWPIARVTMAVQEHVYALPVHEVEKSRPETTE
GSRSAWFKNSGVSNHGVTHAQTLNAILKNAYNVYNGVIKKVENRNAKKRDSLAAKNKSRER
KGLPHFKADPPELATDEQGYLLQPPSPNSSVYLVQQHLRTPQIDLPSGYTGPVVDPRSPIPSLIPI
DRLAIPPGQPGYVPLHDREKLTSNKHRRMKLPKSLRAQGALPVCFRVFDDWAVVDGRGLLR
HAQYRRLAPKNVSIAELLELYTGDPVIDIKRNLMTFRFAEAVVEVTARKIVEKYHNKYLLKLT
EPKGKPVREIGLVSIDLNVQRLIALAIYRVHQTGESQLALSPCLHREILPAKGLGDFDKYKSKF
NQLTEEILTAAVQTLTSAQQEEYQRYVEESSHEAKADLCLKYSITPHELAWDKMTSSTQYISR
WLRDHGWNASDFTQITKGRKKVERLWSDSRWAQELKPKLSNETRRKLEDAKHDLQRANPE
WQRLAKRKQEYSRHLANTVLSMAREYTACETVVIAIENLPMKGGFVDGNGSRESGWDNFFT
HKKENRWMIKDIHKALSDLAPNRGVHVLEVNPQYTSQTCPECGHRDKANRDPIQRERFCCTH
CGAQRHADLEVATHNIAMVATTGKSLTGKSLAPQRLQ (SEQ ID NO: 84)
CasΦ.41
VLLSDRIQYTDPSAPIPAMTVVDRRKIKKGEPGYVPPFMRKNLSTNKHRRMRLSRGQKEACAL
PVGLRLPDGKDGWDFIIFDGRALLRACRRLRLEVTSMDDVLDKFTGDPRIQLSPAGETIVTCM
LKPQHTGVIQQKLITGKMKDRLVQLTAEAPIAMLTVDLGEHNLVACGAYTVGQRRGKLQSER
LEAFLLPEKVLADFEGYRRDSDEHSETLRHEALKALSKRQQREVLDMLRTGADQARESLCYK
YGLDLQALPWDKMSSNSTFIAQHLMSLGFGESATHVRYRPKRKASERTILKYDSRFAAEEKIK
LTDETRRAWNEAIWECQRASQEFRCLSVRKLQLARAAVNWTLTQAKQRSRCPRVVVVVEDL
NVRFMHGGGKRQEGWAGFFKARSEKRWFIQALHKAYTELPTNRGIHVMEVNPARTSITCTKC
GYCDPENRYGEDFHCRNPKCKVRGGHVANADLDIATENLARVALSGPMPKAPKLK (SEQ ID
NO: 85)
CasΦ.42
LEIPEGEPGHVPWFQRMDIPEGQIGHVNKIQRFNFVHGKNSGKVKFSDKTGRVKRYHHSKYK
DATKPYKFLEESKKVSALDSILAIITIGDDWVVFDIRGLYRNVFYRELAQKGLTAVQLLDLFTG
DPVIDPKKGIITFSYKEGVVPVFSQKIVSRFKSRDTLEKLTSQGPVALLSVDLGQNEPVAARVC
SLKNINDKIALDNSCRIPFLDDYKKQIKDYRDSLDELEIKIRLEAINSLDVNQQVEIRDLDVFSA
DRAKASTVDMFDIDPNLISWDSMSDARFSTQISDLYLKNGGDESRVYFEINNKRIKRSDYNISQ
LVRPKLSDSTRKNLNDSIWKLKRTSEEYLKLSKRKLELSRAVVNYTIRQSKLLSGINDIVIILED
LDVKKKFNGRGIRDIGWDNFFSSRKENRWFIPAFHKSFSELSSNRGLCVIEVNPAWTSATCPDC
GFCSKENRDGINFTCRKCGVSYHADIDVATLNIARVAVLGKPMSGPADRERLGGTKKPRVAR
SRKDMKRKDISNGTVEVMVTA (SEQ ID NO: 86)
CasΦ.43
MSEITDLLKANFKGKTFKSADMRMAGRILKKSGAQAVIKYLSDKGAVDPPDFRPPAKCNIIAQ
SRPFDEWPICKASMAIQQHIYGLTKNEFDESSPGTSSASHEQWFAKTGVDTHGFTHVQGLNLIF
QHAKKRYEGVIKKVENYNEKERKKFEGINERRSKEGMPLLEPRLRTAFGDDGKFAEKPGVNP
SIYLYQQTSPRPYDKTKHPYVHAPFELKEITTIPTQDDRLKIPFGAPGHVPEKHRSQLSMAKHK
RRRAWYALSQNKPRPPKDGSKGRRSVRDLADLKAASLADAIPLVSRVGFDWVVIDGRGLLR
NLRWRKLAHEGMTVEEMLGFFSGDPVIDPRRNVATFIYKAEHATVKSRKPIGGAKRAREELL
KATASSDGVIRQVGLISVDLGQTNPVAYEISRMHQANGELVAEHLEYGLLNDEQVNSIQRYR
AAWDSMNESFRQKAIESLSMEAQDEIMQASTGAAKRTREAVLTMFGPNATLPWSRMSSNTT
CISDALIEVGKEEETNFVTSNGPRKRTDAQWAAYLRPRVNPETRALLNQAVWDLMKRSDEYE
RLSKRKLEMARQCVNFVVARAEKLTQCNNIGIVLENLVVRNFHGSGRRESGWEGFFEPKREN
RWFMQVLHKAFSDLAQHRGVMVFEVHPAYSSQTCPACRYVDPKNRSSEDRERFKCLKCGRS
FNADREVATFNIREIARTGVGLPKPDCERSRGVQTTGTARNPGRSLKSNKNPSEPKRVLQSKT
RKKITSTETQNEPLATDLKT (SEQ ID NO: 87)
CasΦ.44
MTPKTESPLSALCKKHFPGKRFRTNYLKDAGKILKKHGEDAVVAFLSDKQEDEPANFCPPAK
VHILAQSRPFEDWPINLASKAIQTYVYGLTADERKTCEPGTSKESHDRWFKETGVDHHGFTSV
QGLNLIFKHTLNRYDGVIKKVETRNEKRRSSVVRINEKKAAEGLPLIAAEAEETAFGEDGRLL
QPPGVNHSIYCFQQVSPQPYSSKKHPQVVLPHAVQGVDPDAPIPVGRPNRLDIPKGQPGYVPE
WQRPHLSMKCKRVRMWYARANWRRKPGRRSVLNEARLKEASAKGALPIVLVIGDDWLVM
DARGLLRSVFWRRVAKPGLSLSELLNVTPTGLFSGDPVIDPKRGLVTFTSKLGVVAVHSRKPT
RGKKSKDLLLKMTKPTDDGMPRHVGMVAIDLGQTNPVAAEYSRVVQSDAGTLKQEPVSRGV
LPDDLLKDVARYRRAYDLTEESIRQEAIALLSEGHRAEVTKLDQTTANETKRLLVDRGVSESL
PWEKMSSNTTYISDCLVALGKTDDVFFVPKAKKGKKETGIAVKRKDHGWSKLLRPRTSPEAR
KALNENQWAVKRASPEYERLSRRKLELGRRCVNHIIQETKRWTQCEDIVVVLEDLNVGFFHG
SGKRPDGWDNFFVSKRENRWFIQVLHKAFGDLATHRGTHVIEVHPARTSITCIKCGHCDAGN
RDGESFVCLASACGDRRHADLEVATRNVARVAITGERMPPSEQARDVQKAGGARKRKPSAR
NVKSSYPAVEPAPASP (SEQ ID NO: 88)
CasΦ.45
QAVIKYLSDKGAVDPPDFRPPAKCNIIAQSRPFDEWPICKASMAIQQHIYGLTKNEFDESSPGTS
SASHEQWFAKTGVDTHGFTHVQGLNLIFQHAKKRYEGVIKKVENYNEKERKKFEGINERRSK
EGMPLLEPRLRTAFGDDGKFAEKPGVNPSIYLYQQTSPRPYDKTKHPYVHAPFELKEITTIPTQ
DDRLKIPFGAPGHVPEKHRSQLSMAKHKRRRAWYALSQNKPRPPKDGSKGRRSVRDLADLK
AASLADAIPLVSRVGFDWVVIDGRGLLRNLRWRKLAHEGMTVEEMLGFFSGDPVIDPRRNVA
TFIYKAEHATVKSRKPIGGAKRAREELLKATASSDGVIRQVGLISVDLGQTNPVAYEISRMHQ
ANGELVAEHLEYGLLNDEQVNSIQRYRAAWDSMNESFRQKAIESLSMEAQDEIMQASTGAA
KRTREAVLTMFGPNATLPWSRMSSNTTCISDALIEVGKEEETNFVTSNGPRKRTDAQWAAYL
RPRVNPETRALLNQAVWDLMKRSDEYERLSKRKLEMARQCVNFVVARAEKLTQCNNIGIVL
ENLVVRNFHGSGRRESGWEGFFEPKRENRWFMQVLHKAFSDLAQHRGVMVFEVHPAYSSQT
CPACRYVDPKNRSSEDRERFKCLKCGRSFNADREVATFNIREIARTGVGLPKPDCERSRDVQT
PGTARKSGRSLKSQDNLSEPKRVLQSKTRKKITSTETQNEPLATDLKT (SEQ ID NO: 89)
CasΦ.46
IPSFGYLDRLKIAKGQPGYIPEWQRETINPSKKVRRYWATNHEKIRNAIPLVVFIGDDWVIIDGR
GLLRDARRRKLADKNTTIEQLLEMVSNDPVIDSTRGIATLSYVEGVVPVRSFIPIGEKKGREYL
EKSTQKESVTLLSVDIGQINPVSCGVYKVSNGCSKIDFLDKFFLDKKHLDAIQKYRTLQDSLEA
SIVNEALDEIDPSFKKEYQNINSQTSNDVKKSLCTEYNIDPEAISWQDITAHSTLISDYLIDNNIT
NDVYRTVNKAKYKTNDFGWYKKFSAKLSKEAREALNEKIWELKIASSKYKKLSVRKKEIART
IANDCVKRAETYGDNVVVAMESLTKNNKVMSGRGKRDPGWHNLGQAKVENRWFIQAISSA
FEDKATHHGTPVLKVNPAYTSQTCPSCGHCSKDNRSSKDRTIFVCKSCGEKFNADLDVATYNI
AHVAFSGKKLSPPSEKSSATKKPRSARKSKKSRKS (SEQ ID NO: 90)
CasΦ.7
SPIEKLLNGLLVKITFGNDWIICDARGLLDNVQKGIIHKSYFTNKSSLVDLIDLFTCNPIVNYKN
NVVTFCYKEGVVDVKSFTPIKSGPKTQENLIKKLKYSRFQNEKDACVLGVGVDVGVTNPFAIN
GFKMPVDESSEWVMLNEPLFTIETSQAFREEIMAYQQRTDEMNDQFNQQSIDLLPPEYKVEFD
NLPEDINEVAKYNLLHTLNIPNNFLWDKMSNTTQFISDYLIQIGRGTETEKTITTKKGKEKILTI
RDVNWFNTFKPKISEETGKARTEIKRDLQKNSDQFQKLAKSREQSCRTWVNNVTEEAKIKSG
CPLIIFVIEALVKDNRVFSGKGHRAIGWHNFGKQKNERRWWVQAIHKAFQEQGVNHGYPVIL
CPPQYTSQTCPKCNHVDRDNRSGEKFKCLKYGWIGNADLDVGAYNIARVAITGKALSKPLEQ
KKIKKAKNKT (SEQ ID NO: 91)
CasΦ.48
LLDNVQKGIIHKSYFTNKSSLVDLIDLFTCNPIVNYKNNVVTFCYKEGVVDVKSFTPIKSGPKT
QENLIKKLKYSRFQNEKDACVLGVGVDVGVTNPFAINGFKMPVDESSEWVMLNEPLFTIETSQ
AFREEIMAYQQRTDEMNDQFNQQSIDLLPPEYKVEFDNLPEDINEVAKYNLLHTLNIPNNFLW
DKMSNTTQFISDYLIQIGRGTETEKTITTKKGKEKILTIRDVNWFNTFKPKISEETGKARTEIKR
DLQKNSDQFQKLAKSREQSCRTWVNNVTEEAKIKSGCPLIIFVIEALVKDNRVFSGKGHRAIG
WHNFGKQKNERRWWVQAIHKAFQEQGVNHGYPVILCPPQYTSQTCPKCNHVDRDNRSGEKF
KCLKYGWIGNADLDVGAYNIARVAITGKALSKPLEQKKIKKAKNKT (SEQ ID NO: 92)
CasM.1584
MIKNPSNRHSLPKVIISEVDHEKILEFKIKYEKLARLDRFEVKAMHYEGKEIVFDEVLVNGGLIE
VEYQDDNKTLFVKVGEKSYSIRGKKVGGKQRLLEDRVSKTKVQLELSDGVVDNKGNLRKSR
TERELIVADNIKLYSQIVGREVTTTKEIYLVKRFLAYRSDLLFYYSFVDNFFKVAGNEKELWKI
NFDDATSAQFMGYIPFMVNDNLKNDNAYLKDYVRNDVQIKDDLKKVQTIFSALRHTLLHFNY
EFFEKLFNGEDVGFDFDIGFLNLLIENIDKLNIDAKKEFIDNEKIRLFGENLSLAKVYRLYSDICV
NRVGFNKFINSMLIKDGVENQVLKAEFNRKFGGNAYTIDIHSNQEYKRIYNEHKKLVIKVSTLK
DGQAIRRGNKKISELKEQMKSMTKKNSLARLECKMRLAFGFLYGEYNNYKAFKNNFDTNIKN
SQFDVNDVEKSKAYFLSTYERRKPRTREKLEKVAKDIESLELKTVIANDTLLKFILLMFVFMPQ
ELKGDFLGFVKKYYHDVHSIDDDTKEQEEDVVEAMSTSLKLKILGRNIRSLTLFKYALSSQVN
YNSTDNIFYVEGNRYGKIYKKLGISHNQEEFDKTLVVPLLRYYSSLFKLMNDFEIYSLAKANPT
AVSLQELVDDETSPYKQGNYFNFNKMLRDIYGLTSDEIKSGQVVFMRNKIAHFDTEVLLSKPL
LGQTKMNLQRKDIVSFIEARGDIKELLGYDAINDFRMKVIHLRTKMRVYSDKLQTMMDLLRN
AKTPNDFYNVYKVKGVESINKHLLEVLAQTAEERTVEKQIRDGNEKYDL (SEQ ID NO: 93)
CasM.1730
MLKHKRKNKNSLARVVLSNYDSNNIYEIKIKYEKLAKLDKINIIEMDYDADNNVMFKKVLFN
NKEIDLSHKDKTKINIELDNKKYNISAKKQIGKTHLVVRDKQTSKISRIKKIQDTYYRGKDVFIL
DNNIEILDKKQTKDKFIVTLNDITNDKTTSTEAELIDDTKDIFKKISAKKDLKSSDIYKIKRFISIR
SNFSFYYTFVDNYFKIFHAKKDKNKEELYKIKFKDEINIKPYLENILDNMKNKNGILYDYADDR
EKVLNDLKNIQYVFTEFRHKLAHFDYNFLDNFFSNSVTDQYKQKVNEIKLLDILLDNIDSLNVV
PKQNYIEDETISVFDAKDIKLKRLYTYYIKLTINYPGFKKLINSFFIQDGIENQELKEYINNKEKD
TQVLKELDNKAYYMDISQYRKYKNIYNKHKELVSEKELSSDGQKINSLNQKINKLKIEMKNIT
KPNALNRLIYRLRVAFGFIYKEYATINNFNKSFLQDTKIKRFENISQQDIKNYLDISYQDKGKFF
VKSKKTFKNKTTIKYTFEDLDLTLNEIITQDDIFVKVIFLFSIFMPKELNGDFFGFINMYYHKMK
NISYDTKDIDMLDTISQNMKLKILEQNIKKTYVFKYYLDLDSSIYSKLVQNIKITEDIDSKKYLY
AKIFKYYQHLYKLISDVEIYLLYKYNSKENLSITIDKDELKHRGYYNFQSLLIKNNINKDDAYW
SIVNMRNNLSHQNIDELVGHFCKGCLRKSTTDIAELWLRKDILTITNEIINKIESFKDIKITLGYD
CVNDFTQKVKQYKQKLKASNERLAKKIEEKQNQVVDEKNKEELEKKILNMKNIQKINRYILDI
L (SEQ ID NO: 94)
CasM.1816
MSQLKNPSNKNSLPRIIISDFNEIKINEIKIKYHKLDRLDKIIVKEMEIINNKIFFKKILFNNQIKDIN
SENIELENYILAGEVKPSNTKIILNRDGKEKSFIVYDGFTFKYKPNDKRISETKTNAKYILTIKDK
TRHRESSTQRDILKSSIIETYKQISGFENITSKDIYTIKRYIDFKNEMMFYYTFIDDFFFPITGKNK
QDKKNNFYNYKIKENAKKFISLINYRINDDFKNKNGILYDYLSNKEEIIINDFIHIQTILKDVRHA
IAHFNFDFIQKLFDNEQAFNSKFDGIEILNILFNQKQEKYFEAQTNYIEEETIKILDEKELSFKKLH
SFYSQICQKKPAFNKLINSFIIQDGIENKELKDYISQKYNSKFDYYLDIHTCKIYKDIYNQHKKFV
ADKQFLENQKTDGQKIKKLNDQINQLKTKMNNLTKKNSLKRLEIKFRLAFGFIFTEYQTFKNF
NERFIEDIKANKYSTKIELLDYGKIKEYISITHEEKRFFNYKTFNKKTNKNINKTIFQSLEKETFE
NLVKNDNLIKMMFLFQLLLPRELKGEFLGFILKIYHDLKNIDNDTKPDEKSLSELNISTALKLKI
LVKNIRQINLFNYTISNNTKYEEKEKRFYEEGNQWKDIYKKLYISHDFDIFDIHLIIPIIKYNINLY
KLIGDFEVYLLLKYLERNTNYKTLDKLIEAEELKYKGYYNFTTLLSKAINIALNDKEYHNITHL
RNNTSHQDIQNIISSFKNNKLLEQRENIIELISKESLKKKLHFDPINDFTMKTLQLLKSLEVHSDK
SEKIENLLKKEPLLPNDVYLLYKLKGIEFIKKELISNIGITKYEEKIQEKIAKGVEK (SEQ ID NO:
95)
CasM.1862939
ELCKIDFTDARSSSLIEYFKFAINDNLKNDRGYLKAYVNDVEQIRADLKKVGGKQRNLEDRVS
RTKVQLTLTNHIEDREGKQRVSRTERELIVPQNIKLYSQIVGREVKTTKEIYLIKRFLEYRSDLLF
YYGFVDNFFKVEGNKKELCKIDFTDARSSSLIEYFKFAINDNLKNDRGYLKAYVNDVEQIRTD
LQKVKTIFSKLRHALMHFDYDFFEKLFNGEEVGFDFDIKFLNIMIDKVEKLNIETKKEFIEDEVI
TLFGERLSLKKLYGLFSHIAINRVAFNKFINSFLIKDGIENRALKDFFNDEKGSQAYEIDIHSNAE
YKALYVQHKKLVMATSAMSDGNEIAKKNQEISELKEKMNAITKANSLARLEYKLRLAFGFIY
TEYGDYTAFKNSFDRDVKSAKYKELSVERLKAYYLATFKASKPQSHEKLEEVAKKIDRLSLKQ
LIENETLLKFVLLLFTFMPQELKGEFLGFIKKYYHDKKHIEQDTKEKEEEREGLSTGLKLKVLE
KNIRSLSILKHALSFQVKYNKKDKNFYEEGNLHGKFYKKLAISHNQEEFNKSVYAPLFRYYVA
LYKLINDFEIYSLAQHIVNNETLADQVGKAQFRQRGYFNFRKLVNCTYATAQNSSYNVLIFMR
NDISHLSYEPLFNCPLEEKASYKQKIRGREKIISVKPLSESRAEIVRFIASQTDMKKLLGYDAVN
DFNMKMVQLRRRLSVYANKQETIEKMINKAKTPNDFYNLYKLKGIECINQHLLKVIGVTEAEK
RIEKQIEEGNEKY (SEQ ID NO: 96)
CasM.1862895
MLKKPSNRYALPKVILSTVDHEKILEFKVKYEKLARLDRLVVERMHEDGESVVFDEVIANSGD
LEIAYQDDHRKLLIQAAGKSYTITGKKVGGKKRKLEERISRAKIQLTLTDGQEDQHRRIRATVT
EKALLEPKEDRDIYSKISDRKIKTSKEIYLVKRFLSYRSDLLFYYFFVDNFFKVGNNKQELWKIK
FQNQPELIEYFRFIINDRFKNAKNDKFDNYLKNDKAIQEDLEKIQKVFEKLRHALMHYDYGFFE
KLFGGEDQGFDLDIAFLDNFVKKIDKLNIDTKKEFVDDEKIKIFGEDLNLADLYKLYASISINRV
GFNRVVNEMIIKDGIEKSELKRAFEKKLDKTYALDIHSDPSYKKLYNEHKRLVTEVSTYTDGN
KIKEGNQKIAKLKYEMKEITKKNALVRLECKMRLAFGLIYGRYDTHEAFKNGFDTDLKRGEF
AQIGSEEAIGYFNTTFEKSKPKSKEEIKKIARQIDNLSLSTLIEDDPLMKFIVLMFLFVPRELKGEF
LGFWRKYYHDIHSIDSDAKSDEMPDEVSLSLKLKILTRNIRRLNLFEYSLSEKIKYSPKNTQFYT
DKSPYQKVYKRLKISHNKEEFDKTLLVPLFRYYSILFKLINDFEIYSLAKANPDASSLSELTKTK
HGFRGHYNFTTLMMDAHKVSQGDSKKHFGIRGEIAHINTKDLIYDPLFRKSKMAQQRNDVIDF
VLKYEKEIKAVLGYDAINDFRMKVVQLRTKLKVYSDKTQTIEKLLNEVEAPDDFYVLYKVKG
VEAINKYLLEIVSVTQAEEEIERKIITGNKRYNT (SEQ ID NO: 97)
CasM.1862903
MLKHKRKNKNSLARVVLSNYDSNNIYEIKIKYEKLAKLDKINIIEMDYDADNNVMFKKVLFN
NKEIDLSHKDKTKINIELDNKKYNISAKKQIGKTHLVVRNKQTSKISRIKKIQDTYYRGKDVFIL
DNNIEILDKKQTKDKFIVTLNDITNNKTTSTEAELIDDTKDIFKKISAKKDLKSSDIYKIKRFISIR
SNFSFYYTFVDNYFKIFHAKKDKNKEELYKIKFKDEINIKPYLENILDNMKNKNGILYNYANDR
KKVLNDLRNIQYVFKEFRHKLAHFDYNFLDNFFSNSVEEKYKQKVNEIKLLDILLDNIDSLNVV
PKQNYIEDETISVFDAKDIKLKRLYTYYIKLTINYPGFKKLINSFFIQDGIENQELKEYINNKEKD
TQVLKELDNKAYYMDISQYRKYKNIYNKHKELVSEKELSSDGKKINSLNQKINKLKIDMKNIT
KPNALNRLIYRLRVAFGFIYKEYATINNFNKSFLQDTKTKRFENISQQDIKSYLDISYQDKGKFF
VKSKKTFKNKTTVKYTFEDLDLTLNEIITQDDIFVKVIFLFSIFMPKELNGDFFGFINMYYHKMK
NISYDTKDIDMLDTISQNMKLKILEQNIKKTYVFKYYLDLDSSIYSKLVQNIKITEDIDSKKYLY
AKIFKYYQHLYKLISDVEIYLLYKYNSKENLSITIDKDELKHRGYYNFQSLLIKNNINKDDAYW
SIVNMRNNLSHQNIDELVGHFCKGCLRKSTTDIAELWLRKDILTITNEIINKIESFKDIKITLGYD
CVNDFTQKVKQYKQKLKASNERLAKKIEEKQNQVVDEKNKEELEKNILNMKNIQKINRYILDI
L (SEQ ID NO: 98)
CasM.1862909
MIKKPSNRHALPKVIISKVDNQNILEFKIKYKKLSRLDRVEIKTMHYDDRAIVFDEVIINGGLID
VEYRDNHKTIFVKVGDKSYSISGQKVGGKERLLENRISQTKVQLELKDEATNRVSKTERELIVD
DNIKLYSQIVGRDVKTTKDIYLIKRFLGYRSDLLFYYGFVNNFFHVANNRPEFWKIDENDNRNS
KLIEYFIFTINDHLKNDENYLKDYISDRGQIVDDLENIKHIFSALRHGLMHFDYDFFEALFNGED
IDIKMDNQGNTQPLSSLNIKFLDIMIDKLDKLNIDTKKEFIDAEKITIFGEELSLAKLYRFYAHTA
INRVAFNKLINSFIIENGVENQSLKEYFNQQAGGIAYEIDIHQNREYKNLYNEHKKLVSRVLSIS
DGQEIATLNQKIVELKEQMKQITKINSIKRLEYKLRLAFGFIYTEYKNYEEFKNSFDTDIKNGRF
TPKDEDGNKRAFDSRELEHLKGYYKATLQTQKPQTDEKMEEVSKRVDRLSLKSLIGDDTLLKF
ILLMFTFMPQELKGEFLGFIKKYYHDTKHIDQDTISDSDDTIEEGLSIGLKLKILDKNIRSLSILKH
SLSFQTKYNKKDRSYYEDGNIHGKFFKKLGISHNQEEFNKSVYAPLFRYYSALYKLINDFEIYT
LSLHIVGNETLSDQVNKPQFLSGRYFNFRKLLTQSYNISNNSTHSVIFNAVINMRNDISHLSYEP
LLDCPLNGKKSYKRKIRNQFRTINIKPLVESRKMIIDFITLQTDMQKVLGCDAVNDFTMKIVQL
RTRLKAYANKEQTIEKMITEAKTPNDFYNIYKVKGVEAINKYLLEVIGETQVEKEIREEIERGNI
ANS (SEQ ID NO: 99)
CasM.1862917
MVKNPANRHALPKVIISEVDNNNILEFKIKYEKLARLDKVEVKSMHFDNNKQVVFDEVVINGG
LIEPTYEDKHKKLVVTAGEKSYSIVGQKVGGKPRLLEDRVSKTKVQLELTNYVEDKEGKKRV
SKTERELIVADNIELYSQIVGREVKTTKEIYLIKRFLEYRSDLLFYYGFVDNFFKVAGNGKELW
KIDFTNSDSLHLIEYFKFSINDNLKNDENYLKNYVSDNTKIENDLVKCQNNFNSLRHALMHFD
YDFFEKLFNGEDVGFDFDIEFLNIMIDKVDKLNIDTKKEFIDDEEVTLFGEALSLKKLYGLFSHI
AINRVAFNKLINSFIIEDGIENKELKDFFNNKKESQAYEIDIHSNAEYKALYVQHKKLVMATSA
MTDGDEIAKKNQEISDLKEKMKVITKENSLARLEHKLRLAFGFIYTEYKDYKTFKKHFDQDIK
GAKYKGLNVEKLKEYYETTLKNSKPKTDEKLEDVAKKIDKLSLKELIDDDTLLKFVLLLFIFMP
QELKGDFLGFIKKYYHDKKHIDQDTKDKDTEIEELSTGLKLKVLDKNIRSLSILKHSFSFQVKY
NRKDKNFYEDGNLHGKFYKKLSISHNQEEFNKSVYAPLFRYYSALYKLINDFEIYALAQHVEN
HETLADQVNKSQFIQKSYFNFRKLLDNTDSISQSSSYNTLIVMRNDISHLSYEPLFNYPLDERKS
YKKKTQKGVKTFHVELLYISRAKIIELISLQTDMKKLLGYDAVNDFNMKVVHLRKRLSVYAN
KEESIRKMQADAKTPNDFYNIYKVKGVESINQHLLKVIGVTEAEKSIEKQINEGNKKHNT (SEQ
ID NO: 100)
CasM.1862921
MIKNPSNRYALPKVIISKIDNQNILEFKIKYKKLSKLDIVKVKSMHYDDRAIIFDEVIVNDGLIDV
EYRDNHKTIFVKVGNKSYSISGQKVGGKERLLENRVSKTKVQLELKDKATNRVSKTERELIVD
DNIKIYSQIVGRDVKTTKDIYLIKRFLAYRSDLLFYYGFVNNFFHVANNRSEFWKIDFNDSNNS
KLIEYFKFTINDHLKNDENYLKDYISDNEKLKNDLIKVKNSFEKIRHALMHFDYDFFVKLENGE
DVGLELDIEFLDIMIDKLDKLNIDTKKEFIDDEKITIFGEELSLAKLYRFYAHTAINRVAFNKLIN
SFIIENGVENQSLKEYFNQQAGGIAYEIDIHQNREYKNLYNEHKKLVSRVLSISDGQEIAILNQKI
AKLKDQMKQITKANSIKRLEYKLRLALGFIYTEYENYEEFKNNFDTDIKNGRFTPKDNDGNKR
AFDSRELEQLKGYYEATIQTQKPKTDEKIEEVSKKIDRLSLKSLIADDILLKFILLMFTFMPQELK
GEFLGFIKKYYHDTKHIDQDTISDSDDTIETLSIGLKLKILDKNIRSLSILKHSLSFQTKYNKKDR
NYYEDGNIHGKFFKKLGISHNQEEFNKSVYAPLFRYYSALYKLINDFEIYTLSLHIVGSETLTDQ
VNKSQFLSGRYFNFRKLLTQSYHINNNSTHSTIFNAVINMRNDISHLSYEPLFDCPLNGKKSYK
RKIRNQFKTINIKPLVESRKIIIDFITLQTDMQKVLGYDAVNDFTMKIVQLRTRLKAYANKEQTI
QKMITEAKTPNDFYNIYKVQGVEEINKYLLEVIGETQAEKEIREKIERGNIANF (SEQ ID NO:
101)
CasM.1862947
MTKKPSNRNSLPKVIINKVDESSILEFKIKYEKLARLDRFEVRSMRYDGDGRIIFDEVVANAGLL
DVDYEDDNRTIVVKIENKAYNIYGKKVGGEKRLNGKISKAKVQLILTDSIRKNANDTHRHSLT
ERELINKNEVDLYSKIAEREISTTKDIYLVKRFLAYRSDLLLYYAFINHYVRVNGNKKEFWKTE
IDDKIIDYFIYTINDTLKNKEGYLEKYIVDRDQIKKDLEKIKQIFSHLRHKLMHYDFRFFTDLFD
GKDVDIKVDNSIQKISELLDIEFLNIVIDKLEKLNIDAKKEFIDDEKITLFGQEIELKKLYSLYAHT
SINRVAFNKLINSFLIKDGVENKELKEYFNAHNQGKESYYIDIHQNQEYKKLYIEHKNLVAKLS
ATTDGKEIAKINRELADKKEQMKQITKANSLKRLEYKLRLAFGFIYTEYKDYERFKNSFDTDT
KKKKFDAIDNAKIIEYFEATNKAKKIEKLEEILKGIDKLSLKTLIQDDILLKFLLLFFTFLPQEIKG
EFLGFIKKYYHDITSLDEDTKDKDDEITELPRSLKLKIFSKNIRKLSILKHSLSYQIKYNKKESSY
YEAGNVFNKMFKKQAISHNLEEFGKSIYLPMLKYYSALYKLINDFEIYALYKDMDTSETLSQQ
VDKQEYKRNEYFNFETLLRKKFGNDIEKVLVTYRNKIAHLDFNFLYDKPINKFISLYKSREKIV
NYIKNHDIQAVLKYDAVNDFVMKVIQLRTKLKVYADKEQTIESMIQNTQNPNGFYNIYKVKA
VENINRHLLKVIGYTESEKAVEEKIRAGNTSKS (SEQ ID NO: 102)
CasM.1422
MEKIKKPSNRNSIPSIIISDYDANKIKEIKVKYLKLARLDKITIQDMEIVDNIVEFKKILLNGVEHT
IIDNQKIEFDNYEITGCIKPSNKRRDGRISQAKYVVTITDKYLRENEKEKRFKSTERELPNNTLLS
RYKQISGFDTLTSKDIYKIKRYIDFKNEMLFYFQFIEEFFNPLLPKGKNFYDLNIEQNKDKVAKFI
VYRLNDDFKNKSLNSYITDTCMIINDFKKIQKILSDFRHALAHFDFDFIQKFFDDQLDKNKFDIN
TISLIETLLDQKEEKNYQEKNNYIDDNDILTIFDEKGSKFSKLHNFYTKISQKKPAFNKLINSFLS
QDGVPNEEFKSYLVTKKLDFFEDIHSNKEYKKIYIQHKNLVIKKQKEESQEKPDGQKLKNYND
ELQKLKDEMNTITKQNSLNRLEVKLRLAFGFIANEYNYNFKNFNDEFTNDVKNEQKIKAFKNS
SNEKLKEYFESTFIEKRFFHFSVNFFNKKTKKEETKQKNIFNSIENETLEELVKESPLLQIITLLYL
FIPRELQGEFVGFILKIYHHTKNITSDTKEDEISIEDAQNSFSLKFKILAKNLRGLQLFHYSLSHNT
LYNNKQCFFYEKGNRWQSVYKSFQISHNQDEFDIHLVIPVIKYYINLNKLMGDFEIYALLKYA
DKNSITVKLSDITSRDDLKYNGHYNFATLLFKTFGIDTNYKQNKVSIQNIKKTRNNLAHQNIEN
MLKAFENSEIFAQREEIVNYLQTEHRMQEVLHYNPINDFTMKTVQYLKSLSVHSQKEGKIADI
HKKESLVPNDYYLIYKLKAIELLKQKVIEVIGESEDEKKIKNAIAKEEQIKKGNN (SEQ ID NO:
103)
CasM.1740
MMTKKPANRHALPKVIISEVDNTNILEFKIKYEKLARLDRVEVKAMHYEDGRIIFDEVVVNGG
LIEVEYQDDHKTLFVQVGEKSYSISGQKVGGKQRLLEDRVSKTKVQLELSDGSSERVSRTERE
LIVADNIKLYSQIVGHEVKTTKEIYLAKRFLGYRSDLLFYYGFVDNFFRESKNLKYGKQPVEL
WEDKFQVNDKLTAYTKFMFNDDLQNSESYLKEYVKDNHKIKNDLESARDIFATFRHNLMHFN
YSFFTRLFNGEDVKIKNLQTKKFESLSDVLRNVEFLNKVIQSIDKLNIDTRKEFIDKEKITLFNEE
LDLQQLYGFFAYTAINRVAFNKLINSFIIKDGIENEQLKEYFNQRVDGTAYEIDIHQNREYKELY
KKHKNLVSKVSTLSDGKEIARGNTEISVLKEQMNKITKANSLKRLEHKLRLAFGFIYTEYGSYK
AFVSRFNEDTKRKKIKNVEFEKIGVEKQKEYYESTFTSNNKDKLGELIQEYEKLSLNDLIENDT
FLKVILLLFIFMPKEVKGDFLGFIKKYYHDTKHIEEDTKEKDEGFTNTLPIGLKLKIVERNIAKLS
VLKHSLSLKVKYNRGQYEEDNTYRKVFKKLNISHNQEEFHKSMFSPLLRYYASLYKLINDFEI
YTLSHYITDKYSTLNKVIASEQFHYRYGWNREEKKGELVKTDNYTFSTLLSKKYGHKNSQEIS
EMRNKISHFDEKILFKFPLEEVSSVPKGKGKYKKDEPIKSLKEKREEIVSLMEKQTDMQKVLGY
DAINDFRMKTVQFQTKLKVYSNKEETIKKMIVEAKTPNDYYNIYKVKGVEGINEHLLNVIGET
EAEKSIQEQIAEGNKVNV (SEQ ID NO: 104)
CasM.280852
MAGKKKDKDVINKTLSVRIIRPRYSDDIEKEISDEKAKRKQDGKTGELDRAFFSELKSRNPDIIT
NDELFPLFTEIQKNLTEIYNKSISLLYMKLIVEEEGGSTASALSAGPYKECKARFNSYISLGLRQK
IQSNFRRKELKGFQVSLPTAKSDRFPIPFCHQVENGKGGFKVYETGDDFIFEVPLIKYTATNKKS
TSGKNYTKVQLNNPPVPMNVPLLLSTMRRRQTKKGMQWNKDEGTNAELRRVMSGEYKVSY
AEIIRRTRFGKHDDWFVNFSIKFKNKTDELNQNVRGGIDIGVSNPLVCAVINGLDRYIVANNDI
MAFNERAMARRRTLLRKNRFKRSGHGAKNKLEPITVLTEKNERFRKSILQRWAREVAEFFKR
TSASVVNMEDLSGITEREDFFSTKLRTTWNYRLMQTTIENKLKEYGIAVNYISPKYTSQTCHSC
GKRNDYFTFSYRSENNYPPFECKECNKVKCNADFNAAKNIALKVVL (SEQ ID NO: 105)
CasM.292901
MRISKTLSLRIVRPFYTPEVEAGIKAEKDKREAQGQTRSLDAKFFNELKKKHSEIILSSEFYSLLS
EVQRQLTSIYNHAMSNLYHKIIVEGEKTSTSKALSNIGYDECKAIFPSYMALGLRQKIQSNFRRR
DLKNFRMAVPTAKSDKFPIPIYRQVDGSKGGFKISENDGKDFIVELPLVDYVAEEVKTAKGRFT
KINISKPPKIKNIPVILSTLRRRQSGQWFSDDGTNAEIRRVISGEYKVSWIEIVRRTRFGKHDDWF
VNMVIKYDKPEEGLDSKVVGGIDVGVSSPLVCALNNSLDRYFVKSSDIIAFNKRAMARRRTLL
RQNKYKRSGHGSKNKLEPITVLTEKNERFKKSIMQRWAKEVAEFFRGKGASVVRMEELSGLK
EKDNFFSSYLRMYWNYGQLQQIIENKLKEYGIKVNYVSPKDTSKKCHSCTHINEFFTFEYRQK
NNFPLFKCEKCGVECSADYNAAKNMAIA (SEQ ID NO: 106)
CasM.294601
MKDYIRKTLSLRILRPYYGEEIEKEIAAAKKKSQAEGGDGALDNKFWDRLKAEHPEIISSREFY
DLLDAIQRETTLYYNRAISKLYHSLIVEREQVSTAKALSAGPYHEFREKFNAYISLGLREKIQSN
FRRKELARYQVALPTAKSDTFPIPIYKGFDKNGKGGFKVREIENGDFVIDLPLMAYHRVGGKA
GREYIELDRPPAVLNVPVILSTSRRRANKTWFRDEGTDAEIRRVMAGEYKVSWVEILQRKRFG
KPYGGWYVNFTIKYQPRDYGLDPKVKGGIDIGLSSPLVCAVTNSLARLTIRDNDLVAFNRKAM
ARRRTLLRQNRYKRSGHGSANKLKPIEALTEKNELYRKAIMRRWAREAADFFRQHRAATVN
MEDLTGIKDREDYFSQMLRCYWNYSQLQTMLENKLKEYGIAVKYIEPKDTSKTCHSCGHVNE
YFDFNYRSAHKFPMFKCEKCGVECGADYNAARNIAQA (SEQ ID NO: 107)
VKISKTLSLRIIRPYYTPEVESAIKAEKDKREAQGQTRNLDAKFFNELKKKHPQIILSGEFYSLLF
EMQRQLTSIYNRAMSSLYHKIIVEGEKTSTSKALSDIGYDECKSVFPSYIALGLRQKIQSNFRRK
ELKGFRMAVPTAKSDKFPIPIYKQVDDGKGGFKISENKEGDFIVELPLVEYTAEDVKTAKGKFT
KINISKPPKIKNIPVILSTLRRKQSGQWFSDEGTNAEIRRVISGEYKVSWIEVVRRTRFGKHDDW
FLNIVIKYDKTEDGLDPEVVGGIDVGVSTPLVCAVNNSLDRYFVKSSDIIAFKKRAMARRRTLL
RQNRFKRSGHGSKSKLEPITILTEKNERFKKSIMQRWAKEVAEFFKGERASVVQMEELSGLKE
KDNFFGSYLRMYWNYGQLQQIIENKLKEYGIKVNYVSPKDTSKKCHSCGYINEFFTFEFRQKN
NFPLFKCKKCGVECNADYNAAKNIAIA (SEQ ID NO: 108)
CasM.298142
VPITKTISLRILRPYYPPEIEAKIKAEKEKRKENGDTGSLNSSYYRELKKEYPSIIINDEFFPLLSEM
QRNITSIYNRTISHLYHRLIIKKESISTAKALSEGPYRDFKSTFNSYIALGLRQKVQSNFRKKDLM
AFKIALPTAKSDKFPIPIYMQTNFKIKESPDSDFIIELPLVEYIAKETKGKNKMFTKVEILSPPKVK
NIPVILSTRRRKESGQWFSDEGTNAEIRRIISGEYKVSWIEIVKRTRFGKHDWFVNMVISFEESQ
EGLDPDVIGGIDIGVSKPLICAINNSLDRYIVKGDDIIAFNRRALSRRRSLLRRNRLKRSGHGSRN
KLEPITVLTEKNERFKKSIMQRWAKEVAEFFKSKRASIVQMEELTGIKEREDFFSKTLRMYWN
YGQLQKTVENKLREYGIEVRYASPKDTSRRCHSCGHINDYFTFEFRQQNNFPLFKCMNCGIEC
SADYNAARNIAIAR (SEQ ID NO: 109)
MEVQKTVMKTLSLRILRPLYSQEIEKEIKEEKERRKQAGGTGELDGGFYKKLEKKHSEMFSFD
RLNLLLNQLQREIAKVYNHAISELYIATIAQGNKSNKHYISSIVYNRAYGYFYNAYIALGICSKV
EANFRSNELLTQQSALPTAKSDNFPIVLHKQKGAEGEDGGFRISTEGSDLIFEIPIPFYEYNGENR
KEPYKWVKKGGQKPVLKLILSTFRRQRNKGWAKDEGTDAEIRKVTEGKYQVSQIEINRGKKL
GEHQKWFANFSIEQPIYERKPNRSIVGGLDVGIRSPLVCAINNSFSRYSVDSNDVFKFSKQVFAF
RRRLLSKNSLKRKGHGAAHKLEPITEMTEKNDKFRKKIIERWAKEVTNFFVKNQVGIVQIEDLS
TMKDREDHFFNQYLRGFWPYYQMQTLIENKLKEYGIEVKRVQAKYTSQLCSNPNCRYWNNY
FNFEYRKVNKFPKFKCEKCNLEISADYNAARNLSTPDIEKFVAKATKGINLPEK (SEQ ID NO:
110)
MEEAKTVSKTLSLRILRPLYSAEIEKEIKEEKERRKQGGKSGELDSGFYKKLEKKHTQMFGWD
KLNLMLSQLQRQIARVFNQSISELYIETVIQGKKSNKHYTSKIVYNRAYSVFYNAYLALGITSK
VEANFRSTELLMQKSSLPTAKSDNFPILLHKQKGVEGEEGGFKISADGNDLIFEIPIPFYEYDSA
NKKEPFKWIKKGGQKPTIKLILSTFRRQRNKGWAKDEGTDAEIRKVIEGKYQVSHIEINRGKKL
GDHQKWFVNFTIEQPIYERKLDKNIIGGIDVGIKSPLVCAVNNSFARYSVDSNDVLKFSKQAFA
FRRRLLSKNSLKRSGHGSKNKLDPITRMTEKNDRFRKKIIERWAKEVTNFFIKNQVGTVQIEDL
STMKDRQDNFFNQYLRGFWPYYQMQNLIENKLKEYGIETKRIKARYTSQLCSNPSCRHWNSY
FSFDHRKTNNFPKFKCEKCALEISADYNAARNISTPDIEKFVAKATKGINLPDKNENVILE (SEQ
ID NO: 111)
Cas14a.1
MAKNTITKTLKLRIVRPYNSAEVEKIVADEKNNREKIALEKNKDKVKEACSKHLKVAAYCTT
QVERNACLFCKARKLDDKFYQKLRGQFPDAVFWQEISEIFRQLQKQAAEIYNQSLIELYYEIFI
KGKGIANASSVEHYLSDVCYTRAAELFKNAAIASGLRSKIKSNFRLKELKNMKSGLPTTKSDN
FPIPLVKQKGGQYTGFEISNHNSDFIIKIPFGRWQVKKEIDKYRPWEKFDFEQVQKSPKPISLLLS
TQRRKRNKGWSKDEGTEAEIKKVMNGDYQTSYIEVKRGSKIGEKSAWMLNLSIDVPKIDKGV
DPSIIGGIDVGVKSPLVCAINNAFSRYSISDNDLFHFNKKMFARRRILLKKNRHKRAGHGAKNK
LKPITILTEKSERFRKKLIERWACEIADFFIKNKVGTVQMENLESMKRKEDSYFNIRLRGFWPY
AEMQNKIEFKLKQYGIEIRKVAPNNTSKTCSKCGHLNNYFNFEYRKKNKFPHFKCEKCNFKEN
ADYNAALNISNPKLKSTKEEP (SEQ ID NO: 112)
MERQKVPQIRKIVRVVPLRILRPKYSDVIENALKKFKEKGDDTNTNDFWRAIRDRDTEFFRKEL
NFSEDEINQLERDTLFRVGLDNRVLFSYFDFLQEKLMKDYNKIISKLFINRQSKSSFENDLTDEE
VEELIEKDVTPFYGAYIGKGIKSVIKSNLGGKFIKSVKIDRETKKVTKLTAINIGLMGLPVAKSD
TFPIKIIKTNPDYITFQKSTKENLQKIEDYETGIEYGDLLVQITIPWFKNENKDFSLIKTKEAIEYY
KLNGVGKKDLLNINLVLTTYHIRKKKSWQIDGSSQSLVREMANGELEEKWKSFFDTFIKKYGD
EGKSALVKRRVNKKSRAKGEKGRELNLDERIKRLYDSIKAKSFPSEINLIPENYKWKLHFSIEIP
PMVNDIDSNLYGGIDFGEQNIATLCVKNIEKDDYDFLTIYGNDLLKHAQASYARRRIMRVQDE
YKARGHGKSRKTKAQEDYSERMQKLRQKITERLVKQISDFFLWRNKFHMAVCSLRYEDLNTL
YKGESVKAKRMRQFINKQQLFNGIERKLKDYNSEIYVNSRYPHYTSRLCSKCGKLNLYFDFLK
FRTKNIIIRKNPDGSEIKYMPFFICEFCGWKQAGDKNASANIADKDYQDKLNKEKEFCNIRKPK
SKKEDIGEENEEERDYSRRFNRNSFIYNSLKKDNKLNQEKLFDEWKNQLKRKIDGRNKFEPKE
YKDRFSYLFAYYQEIIKNESES (SEQ ID NO: 113)
MVPTELITKTLQLRVIRPLYFEEIEKELAELKEQKEKEFEETNSLLLESKKIDAKSLKKLKRKAR
SSAAVEFWKIAKEKYPDILTKPEMEFIFSEMQKMMARFYNKSMTNIFIEMNNDEKVNPLSLISK
ASTEANQVIKCSSISSGLNRKIAGSINKTKFKQVRDGLISLPTARTETFPISFYKSTANKDEIPISKI
NLPSEEEADLTITLPFPFFEIKKEKKGQKAYSYFNIIEKSGRSNNKIDLLLSTHRRQRRKGWKEE
GGTSAEIRRLMEGEFDKEWEIYLGEAEKSEKAKNDLIKNMTRGKLSKDIKEQLEDIQVKYFSD
NNVESWNDLSKEQKQELSKLRKKKVEELKDWKHVKEILKTRAKIGWVELKRGKRQRDRNK
WFVNITITRPPFINKELDDTKFGGIDLGVKVPFVCAVHGSPARLIIKENEILQFNKMVSARNRQIT
KDSEQRKGRGKKNKFIKKEIFNERNELFRKKIIERWANQIVKFFEDQKCATVQIENLESFDRTSY
K (SEQ ID NO: 114)
MKSDTKDKKIIIHQTKTLSLRIVKPQSIPMEEFTDLVRYHQMIIFPVYNNGAIDLYKKLFKAKIQ
KGNEARAIKYFMNKIVYAPIANTVKNSYIALGYSTKMQSSFSGKRLWDLRFGEATPPTIKADFP
LPFYNQSGFKVSSENGEFIIGIPFGQYTKKTVSDIEKKTSFAWDKFTLEDTTKKTLIELLLSTKTR
KMNEGWKNNEGTEAEIKRVMDGTYQVTSLEILQRDDSWFVNFNIAYDSLKKQPDRDKIAGIH
MGITRPLTAVIYNNKYRALSIYPNTVMHLTQKQLARIKEQRTNSKYATGGHGRNAKVTGTDT
LSEAYRQRRKKIIEDWIASIVKFAINNEIGTIYLEDISNTNSFFAAREQKLIYLEDISNTNSFLSTY
KYPISAISDTLQHKLEEKAIQVIRKKAYYVNQICSLCGHYNKGFTYQFRRKNKFPKMKCQGCL
EATSTEFNAAANVANPDYEKLLIKHGLLQLKK (SEQ ID NO: 115)
MSTITRQVRLSPTPEQSRLLMAHCQQYISTVNVLVAAFDSEVLTGKVSTKDFRAALPSAVKNQ
ALRDAQSVFKRSVELGCLPVLKKPHCQWNNQNWRVEGDQLILPICKDGKTQQERFRCAAVAL
EGKAGILRIKKKRGKWIADLTVTQEDAPESSGSAIMGVDLGIKVPAVAHIGGKGTRFFGNGRS
QRSMRRRFYARRKTLQKAKKLRAVRKSKGKEARWMKTINHQLSRQIVNHAHALGVGTIKIEA
LQGIRKGTTRKSRGAAARKNNRMTNTWSFSQLTLFITYKAQRQGITVEQVDPAYTSQDCPACR
ARNGAQDRTYVCSECGWRGHRDTVGAINISRRAGLSGHRRGATGA (SEQ ID NO: 116)
MIAQKTIKIKLNPTKEQIIKLNSIIEEYIKVSNFTAKKIAEIQESFTDSGLTQGTCSECGKEKTYRK
YHLLKKDNKLFCITCYKRKYSQFTLQKVEFQNKTGLRNVAKLPKTYYTNAIRFASDTFSGFDEI
IKKKQNRLNSIQNRLNFWKELLYNPSNRNEIKIKVVKYAPKTDTREHPHYYSEAEIKGRIKRLE
KQLKKFKMPKYPEFTSETISLQRELYSWKNPDELKISSITDKNESMNYYGKEYLKRYIDLINSQ
TPQILLEKENNSFYLCFPITKNIEMPKIDDTFEPVGIDWGITRNIAVVSILDSKTKKPKFVKFYSA
GYILGKRKHYKSLRKHFGQKKRQDKINKLGTKEDRFIDSNIHKLAFLIVKEIRNHSNKPIILMEN
ITDNREEAEKSMRQNILLHSVKSRLQNYIAYKALWNNIPTNLVKPEHTSQICNRCGHQDRENRP
KGSKLFKCVKCNYMSNADFNASINIARKFYIGEYEPFYKDNEKMKSGVNSISM (SEQ ID NO:
117)
LKLSEQENITTGVKFKLKLDKETSEGLNDYFDEYGKAINFAIKVIQKELAEDRFAGKVRLDENK
KPLLNEDGKKIWDFPNEFCSCGKQVNRYVNGKSLCQECYKNKFTEYGIRKRMYSAKGRKAEQ
DINIKNSTNKISKTHFNYAIREAFILDKSIKKQRKERFRRLREMKKKLQEFIEIRDGNKILCPKIEK
QRVERYIHPSWINKEKKLEDFRGYSMSNVLGKIKILDRNIKREEKSLKEKGQINFKARRLMLDK
SVKFLNDNKISFTISKNLPKEYELDLPEKEKRLNWLKEKIKIIKNQKPKYAYLLRKDDNFYLQY
TLETEFNLKEDYSGIVGIDRGVSHIAVYTFVHNNGKNERPLFLNSSEILRLKNLQKERDRFLRRK
HNKKRKKSNMRNIEKKIQLILHNYSKQIVDFAKNKNAFIVFEKLEKPKKNRSKMSKKSQYKLS
QFTFKKLSDLVDYKAKREGIKVLYISPEYTSKECSHCGEKVNTQRPFNGNSSLFKCNKCGVEL
NADYNASINIAKKGLNILNSTN (SEQ ID NO: 118)
MEESIITGVKFKLRIDKETTKKLNEYFDEYGKAINFAVKIIQKELADDRFAGKAKLDQNKNPIL
DENGKKIYEFPDEFCSCGKQVNKYVNNKPFCQECYKIRFTENGIRKRMYSAKGRKAEHKINIL
NSTNKISKTHFNYAIREAFILDKSIKKQRKKRNERLRESKKRLQQFIDMRDGKREICPTIKGQKV
DRFIHPSWITKDKKLEDFRGYTLSIINSKIKILDRNIKREEKSLKEKGQIIFKAKRLMLDKSIRFVG
DRKVLFTISKTLPKEYELDLPSKEKRLNWLKEKIEIIKNQKPKYAYLLRKNIESEKKPNYEYYLQ
YTLEIKPELKDFYDGAIGIDRGINHIAVCTFISNDGKVTPPKFFSSGEILRLKNLQKERDRFLLRK
HNKNRKKGNMRVIENKINLILHRYSKQIVDMAKKLNASIVFEELGRIGKSRTKMKKSQRYKLS
LFIFKKLSDLVDYKSRREGIRVTYVPPEYTSKECSHCGEKVNTQRPFNGNYSLFKCNKCGIQLN
SDYNASINIAKKGLKIPNST (SEQ ID NO: 119)
LWTIVIGDFIEMPKQDLVTTGIKFKLDVDKETRKKLDDYFDEYGKAINFAVKIIQKNLKEDRFA
GKIALGEDKKPLLDKDGKKIYNYPNESCSCGNQVRRYVNAKPFCVDCYKLKFTENGIRKRMY
SARGRKADSDINIKNSTNKISKTHFNYAIREGFILDKSLKKQRSKRIKKLLELKRKLQEFIDIRQG
QMVLCPKIKNQRVDKFIHPSWLKRDKKLEEFRGYSLSVVEGKIKIFNRNILREEDSLRQRGHVN
FKANRIMLDKSVRFLDGGKVNFNLNKGLPKEYLLDLPKKENKLSWLNEKISLIKLQKPKYAYL
LRREGSFFIQYTIENVPKTFSDYLGAIGIDRGISHIAVCTFVSKNGVNKAPVFFSSGEILKLKSLQ
KQRDLFLRGKHNKIRKKSNMRNIDNKINLILHKYSRNIVNLAKSEKAFIVFEKLEKIKKSRFKM
SKSLQYKLSQFTFKKLSDLVEYKAKIEGIKVDYVPPEYTSKECSHCGEKVDTQRPFNGNSSLFK
CNKCRVQLNADYNASINIAKKSLNISN (SEQ ID NO: 120)
MSKTTISVKLKIIDLSSEKKEFLDNYFNEYAKATTFCQLRIRRLLRNTHWLGKKEKSSKKWIFES
GICDLCGENKELVNEDRNSGEPAKICKRCYNGRYGNQMIRKLFVSTKKREVQENMDIRRVAK
LNNTHYHRIPEEAFDMIKAADTAEKRRKKNVEYDKKRQMEFIEMFNDEKKRAARPKKPNERE
TRYVHISKLESPSKGYTLNGIKRKIDGMGKKIERAEKGLSRKKIFGYQGNRIKLDSNWVRFDLA
ESEITIPSLFKEMKLRITGPTNVHSKSGQIYFAEWFERINKQPNNYCYLIRKTSSNGKYEYYLQY
TYEAEVEANKEYAGCLGVDIGCSKLAAAVYYDSKNKKAQKPIEIFTNPIKKIKMRREKLIKLLS
RVKVRHRRRKLMQLSKTEPIIDYTCHKTARKIVEMANTAKAFISMENLETGIKQKQQARETKK
QKFYRNMFLFRKLSKLIEYKALLKGIKIVYVKPDYTSQTCSSCGADKEKTERPSQAIFRCLNPT
CRYYQRDINADFNAAVNIAKKALNNTEVVTTLL (SEQ ID NO: 121)
MARAKNQPYQKLTTTTGIKFKLDLSEEEGKRFDEYFSEYAKAVNFCAKVIYQLRKNLKFAGK
KELAAKEWKFEISNCDFCNKQKEIYYKNIANGQKVCKGCHRTNFSDNAIRKKMIPVKGRKVES
KFNIHNTTKKISGTHRHWAFEDAADIIESMDKQRKEKQKRLRREKRKLSYFFELFGDPAKRYE
LPKVGKQRVPRYLHKIIDKDSLTKKRGYSLSYIKNKIKISERNIERDEKSLRKASPIAFGARKIK
MSKLDPKRAFDLENNVFKIPGKVIKGQYKFFGTNVANEHGKKFYKDRISKILAGKPKYFYLLR
KKVAESDGNPIFEYYVQWSIDTETPAITSYDNILGIDAGITNLATTVLIPKNLSAEHCSHCGNNH
VKPIFTKFFSGKELKAIKIKSRKQKYFLRGKHNKLVKIKRIRPIEQKVDGYCHVVSKQIVEMAK
ERNSCIALEKLEKPKKSKFRQRRREKYAVSMFVFKKLATFIKYKAAREGIEIIPVEPEGTSYTCS
HCKNAQNNQRPYFKPNSKKSWTSMFKCGKCGIELNSDYNAAFNIAQKALNMTSA (SEQ ID
NO: 122)
MDEKHFFCSYCNKELKISKNLINKISKGSIREDEAVSKAISIHNKKEHSLILGIKFKLFIENKLDK
KKLNEYFDNYSKAVTFAARIFDKIRSPYKFIGLKDKNTKKWTFPKAKCVFCLEEKEVAYANEK
DNSKICTECYLKEFGENGIRKKIYSTRGRKVEPKYNIFNSTKELSSTHYNYAIRDAFQLLDALKK
QRQKKLKSIFNQKLRLKEFEDIFSDPQKRIELSLKPHQREKRYIHLSKSGQESINRGYTLRFVRG
KIKSLTRNIEREEKSLRKKTPIHFKGNRLMIFPAGIKFDFASNKVKISISKNLPNEFNFSGTNVKN
EHGKSFFKSRIELIKTQKPKYAYVLRKIKREYSKLRNYEIEKIRLENPNADLCDFYLQYTIETESR
NNEEINGIIGIDRGITNLACLVLLKKGDKKPSGVKFYKGNKILGMKIAYRKHLYLLKGKRNKLR
KQRQIRAIEPKINLILHQISKDIVKIAKEKNFAIALEQLEKPKKARFAQRKKEKYKLALFTFKNLS
TLIEYKSKREGIPVIYVPPEKTSQMCSHCAINGDEHVDTQRPYKKPNAQKPSYSLFKCNKCGIE
LNADYNAAFNIAQKGLKTLMLNHSH (SEQ ID NO: 123)
MLQTLLVKLDPSKEQYKMLYETMERFNEACNQIAETVFAIHSANKIEVQKTVYYPIREKFGLS
AQLTILAIRKVCEAYKRDKSIKPEFRLDGALVYDQRVLSWKGLDKVSLVTLQGRQIIPIKFGDY
QKARMDRIRGQADLILVKGVFYLCVVVEVSEESPYDPKGVLGVDLGIKNLAVDSDGEVHSGE
QTTNTRERLDSLKARLQSKGTKSAKRHLKKLSGRMAKFSKDVNHCISKKLVAKAKGTLMSIA
LEDLQGIRDRVTVRKAQRRNLHTWNFGLLRMFVDYKAKIAGVPLVFVDPRNTSRTCPSCGHV
AKANRPTRDEFRCVSCGFAGAADHIAAMNIAFRAEVSQPIVTRFFVQSQAPSFRVG (SEQ ID
NO: 124)
MDEEPDSAEPNLAPISVKLKLVKLDGEKLAALNDYFNEYAKAVNFCELKMQKIRKNLVNIRG
TYLKEKKAWINQTGECCICKKIDELRCEDKNPDINGKICKKCYNGRYGNQMIRKLFVSTNKRA
VPKSLDIRKVARLHNTHYHRIPPEAADIIKAIETAERKRRNRILFDERRYNELKDALENEEKRVA
RPKKPKEREVRYVPISKKDTPSKGYTMNALVRKVSGMAKKIERAKRNLNKRKKIEYLGRRILL
DKNWVRFDFDKSEISIPTMKEFFGEMRFEITGPSNVMSPNGREYFTKWFDRIKAQPDNYCYLL
RKESEDETDFYLQYTWRPDAHPKKDYTGCLGIDIGGSKLASAVYFDADKNRAKQPIQIFSNPIG
KWKTKRQKVIKVLSKAAVRHKTKKLESLRNIEPRIDVHCHRIARKIVGMALAANAFISMENLE
GGIREKQKAKETKKQKFSRNMFVFRKLSKLIEYKALMEGVKVVYIVPDYTSQLCSSCGTNNTK
RPKQAIFMCQNTECRYFGKNINADFNAAINIAKKALNRKDIVRELS (SEQ ID NO: 125)
MEKNNSEQTSITTGIKFKLKLDKETKEKLNNYFDEYGKAINFAVRIIQMQLNDDRLAGKYKRD
EKGKPILGEDGKKILEIPNDFCSCGNQVNHYVNGVSFCQECYKKRFSENGIRKRMYSAKGRKA
EQDINIKNSTNKISKTHFNYAIREAFNLDKSIKKQREKRFKKLKDMKRKLQEFLEIRDGKRVICP
KIEKQKVERYIHPSWINKEKKLEEFRGYSLSIVNSKIKSFDRNIQREEKSLKEKGQINFKAQRLM
LDKSVKFLKDNKVSFTISKELPKTFELDLPKKEKKLNWLNEKLEIIKNQKPKYAYLLRKENNIF
LQYTLDSIPEIHSEYSGAVGIDRGVSHIAVYTFLDKDGKNERPFFLSSSGILRLKNLQKERDKFL
RKKHNKIRKKGNMRNIEQKINLILHEYSKQIVNFAKDKNAFIVFELLEKPKKSRERMSKKIQYK
LSQFTFKKLSDLVDYKAKREGIKVIYVEPAYTSKDCSHCGERVNTQRPFNGNFSLFKCNKCGIV
LNSDYNASLNIARKGLNISAN (SEQ ID NO: 126)
MAEEKFFFCEKCNKDIKIPKNYINKQGAEEKARAKHEHRVHALILGIKFKIYPKKEDISKLNDY
FDEYAKAVTFTAKIVDKLKAPFLFAGKRDKDTSKKKWVFPVDKCSFCKEKTEINYRTKQGKNI
CNSCYLTEFGEQGLLEKIYATKGRKVSSSFNLFNSTKKLTGTHNNYVVKESLQLLDALKKQRS
KRLKKLSNTRRKLKQFEEMFEKEDKRFQLPLKEKQRELRFIHVSQKDRATEFKGYTMNKIKSK
IKVLRRNIEREQRSLNRKSPVFFRGTRIRLSPSVQFDDKDNKIKLTLSKELPKEYSFSGLNVANE
HGRKFFAEKLKLIKENKSKYAYLLRRQVNKNNKKPIYDYYLQYTVEFLPNIITNYNGILGIDRG
INTLACIVLLENKKEKPSFVKFFSGKGILNLKNKRRKQLYFLKGVHNKYRKQQKIRPIEPRIDQI
LHDISKQIIDLAKEKRVAISLEQLEKPQKPKFRQSRKAKYKLSQFNFKTLSNYIDYKAKKEGIRV
IYIAPEMTSQNCSRCAMKNDLHVNTQRPYKNTSSLFKCNKCGVELNADYNAAFNIAQKGLKIL
NS (SEQ ID NO: 127)
MISLKLKLLPDEEQKKLLDEMFWKWASICTRVGFGRADKEDLKPPKDAEGVWFSLTQLNQAN
TDINDLREAMKHQKHRLEYEKNRLEAQRDDTQDALKNPDRREISTKRKDLFRPKASVEKGFL
KLKYHQERYWVRRLKEINKLIERKTKTLIKIEKGRIKFKATRITLHQGSFKIRFGDKPAFLIKALS
GKNQIDAPFVVVPEQPICGSVVNSKKYLDEITTNFLAYSVNAMLFGLSRSEEMLLKAKRPEKIK
KKEEKLAKKQSAFENKKKELQKLLGRELTQQEEAIIEETRNQFFQDFEVKITKQYSELLSKIAN
ELKQKNDFLKVNKYPILLRKPLKKAKSKKINNLSPSEWKYYLQFGVKPLLKQKSRRKSRNVLG
IDRGLKHLLAVTVLEPDKKTFVWNKLYPNPITGWKWRRRKLLRSLKRLKRRIKSQKHETIHEN
QTRKKLKSLQGRIDDLLHNISRKIVETAKEYDAVIVVEDLQSMRQHGRSKGNRLKTLNYALSL
FDYANVMQLIKYKAGIEGIQIYDVKPAGTSQNCAYCLLAQRDSHEYKRSQENSKIGVCLNPNC
QNHKKQIDADLNAARVIASCYALKINDSQPFGTRKRFKKRTTN (SEQ ID NO: 128)
METLSLKLKLNPSKEQLLVLDKMFWKWASICTRLGLKKAEMSDLEPPKDAEGVWFSKTQLN
QANTDVNDLRKAMQHQGKRIEYELDKVENRRNEIQEMLEKPDRRDISPNRKDLFRPKAAVEK
GYLKLKYHKLGYWSKELKTANKLIERKRKTLAKIDAGKMKFKPTRISLHTNSFRIKFGEEPKIA
LSTTSKHEKIELPLITSLQRPLKTSCAKKSKTYLDAAILNFLAYSTNAALFGLSRSEEMLLKAKK
PEKIEKRDRKLATKRESFDKKLKTLEKLLERKLSEKEKSVFKRKQTEFFDKFCITLDETYVEAL
HRIAEELVSKNKYLEIKKYPVLLRKPESRLRSKKLKNLKPEDWTYYIQFGFQPLLDTPKPIKTKT
VLGIDRGVRHLLAVSIFDPRTKTFTFNRLYSNPIVDWKWRRRKLLRSIKRLKRRLKSEKHVHLH
ENQFKAKLRSLEGRIEDHFHNLSKEIVDLAKENNSVIVVENLGGMRQHGRGRGKWLKALNYA
LSHFDYAKVMQLIKYKAELAGVFVYDVAPAGTSINCAYCLLNDKDASNYTRGKVINGKKNTK
IGECKTCKKEFDADLNAARVIALCYEKRLNDPQPFGTRKQFKPKKP (SEQ ID NO: 129)
MKALKLQLIPTRKQYKILDEMFWKWASLANRVSQKGESKETLAPKKDIQKIQFNATQLNQIEK
DIKDLRGAMKEQQKQKERLLLQIQERRSTISEMLNDDNNKERDPHRPLNFRPKGWRKFHTSK
HWVGELSKILRQEDRVKKTIERIVAGKISFKPKRIGIWSSNYKINFFKRKISINPLNSKGFELTLM
TEPTQDLIGKNGGKSVLNNKRYLDDSIKSLLMFALHSRFFGLNNTDTYLLGGKINPSLVKYYK
KNQDMGEFGREIVEKFERKLKQEINEQQKKIIMSQIKEQYSNRDSAFNKDYLGLINEFSEVENQ
RKSERAEYLLDSFEDKIKQIKQEIGESLNISDWDFLIDEAKKAYGYEEGFTEYVYSKRYLEILNK
IVKAVLITDIYFDLRKYPILLRKPLDKIKKISNLKPDEWSYYIQFGYDSINPVQLMSTDKFLGIDR
GLTHLLAYSVFDKEKKEFIINQLEPNPIMGWKWKLRKVKRSLQHLERRIRAQKMVKLPENQM
KKKLKSIEPKIEVHYHNISRKIVNLAKDYNASIVVESLEGGGLKQHGRKKNARNRSLNYALSLF
DYGKIASLIKYKADLEGVPMYEVLPAYTSQQCAKCVLEKGSFVDPEIIGYVEDIGIKGSLLDSLF
EGTELSSIQVLKKIKNKIELSARDNHNKEINLILKYNFKGLVIVRGQDKEEIAEHPIKEINGKFAIL
DFVYKRGKEKVGKKGNQKVRYTGNKKVGYCSKHGQVDADLNASRVIALCKYLDINDPILFG
EQRKSFK (SEQ ID NO: 130)
MVTRAIKLKLDPTKNQYKLLNEMFWKWASLANRFSQKGASKETLAPKDGTQKIQFNATQLN
QIKKDVDDLRGAMEKQGKQKERLLIQIQERLLTISEILRDDSKKEKDPHRPQNFRPFGWRRFHT
SAYWSSEASKLTRQVDRVRRTIERIKAGKINFKPKRIGLWSSTYKINFLKKKINISPLKSKSFELD
LITEPQQKIIGKEGGKSVANSKKYLDDSIKSLLIFAIKSRLFGLNNKDKPLFENIITPNLVRYHKK
GQEQENFKKEVIKKFENKLKKEISQKQKEIIFSQIERQYENRDATFSEDYLRAISEFSEIFNQRKK
ERAKELLNSFNEKIRQLKKEVNGNISEEDLKILEVEAEKAYNYENGFIEWEYSEQFLGVLEKIA
RAVLISDNYFDLKKYPILIRKPTNKSKKITNLKPEEWDYYIQFGYGLINSPMKIETKNFMGIDRG
LTHLLAYSIFDRDSEKFTINQLELNPIKGWKWKLRKVKRSLQHLERRMRAQKGVKLPENQMK
KRLKSIEPKIESYYHNLSRKIVNLAKANNASIVVESLEGGGLKQHGRKKNSRHRALNYALSLFD
YGKIASLIKYKSDLEGVPMYEVLPAYTSQQCAKCVLKKGSFVEPEIIGYIEEIGFKENLLTLLFE
DTGLSSVQVLKKSKNKMTLSARDKEGKMVDLVLKYNFKGLVISQEKKKEEIVEFPIKEIDGKF
AVLDSAYKRGKERISKKGNQKLVYTGNKKVGYCSVHGQVDADLNASRVIALCKYLGINEPIV
FGEQRKSFK (SEQ ID NO: 131)
LDLITEPIQPHKSSSLRSKEFLEYQISDFLNFSLHSLFFGLASNEGPLVDFKIYDKIVIPKPEERFPK
KESEEGKKLDSFDKRVEEYYSDKLEKKIERKLNTEEKNVIDREKTRIWGEVNKLEEIRSIIDEIN
EIKKQKHISEKSKLLGEKWKKVNNIQETLLSQEYVSLISNLSDELTNKKKELLAKKYSKFDDKI
KKIKEDYGLEFDENTIKKEGEKAFLNPDKFSKYQFSSSYLKLIGEIARSLITYKGFLDLNKYPIIF
RKPINKVKKIHNLEPDEWKYYIQFGYEQINNPKLETENILGIDRGLTHILAYSVFEPRSSKFILNK
LEPNPIEGWKWKLRKLRRSIQNLERRWRAQDNVKLPENQMKKNLRSIEDKVENLYHNLSRKI
VDLAKEKNACIVFEKLEGQGMKQHGRKKSDRLRGLNYKLSLFDYGKIAKLIKYKAEIEGIPIYR
IDSAYTSQNCAKCVLESRRFAQPEEISCLDDFKEGDNLDKRILEGTGLVEAKIYKKLLKEKKED
FEIEEDIAMFDTKKVIKENKEKTVILDYVYTRRKEIIGTNHKKNIKGIAKYTGNTKIGYCMKHG
QVDADLNASRTIALCKNFDINNPEIWK (SEQ ID NO: 132)
MSDESLVSSEDKLAIKIKIVPNAEQAKMLDEMFKKWSSICNRISRGKEDIETLRPDEGKELQFNS
TQLNSATMDVSDLKKAMARQGERLEAEVSKLRGRYETIDASLRDPSRRHTNPQKPSSFYPSD
WDISGRLTPRFHTARHYSTELRKLKAKEDKMLKTINKIKNGKIVFKPKRITLWPSSVNMAFKGS
RLLLKPFANGFEMELPIVISPQKTADGKSQKASAEYMRNALLGLAGYSINQLLFGMNRSQKML
ANAKKPEKVEKFLEQMKNKDANFDKKIKALEGKWLLDRKLKESEKSSIAVVRTKFFKSGKVE
LNEDYLKLLKHMANEILERDGFVNLNKYPILSRKPMKRYKQKNIDNLKPNMWKYYIQFGYEPI
FERKASGKPKNIMGIDRGLTHLLAVAVFSPDQQKFLFNHLESNPIMHWKWKLRKIRRSIQHME
RRIRAEKNKHIHEAQLKKRLGSIEEKTEQHYHIVSSKIINWAIEYEAAIVLESLSHMKQRGGKKS
VRTRALNYALSLFDYEKVARLITYKARIRGIPVYDVLPGMTSKTCATCLLNGSQGAYVRGLET
TKAAGKATKRKNMKIGKCMVCNSSENSMIDADLNAARVIAICKYKNLNDPQPAGSRKVFKRF
(SEQ ID NO: 133)
MLALKLKIMPTEKQAEILDAMFWKWASICSRIAKMKKKVSVKENKKELSKKIPSNSDIWFSKT
QLCQAEVDVGDHKKALKNFEKRQESLLDELKYKVKAINEVINDESKREIDPNNPSKFRIKDST
KKGNLNSPKFFTLKKWQKILQENEKRIKKKESTIEKLKRGNIFFNPTKISLHEEEYSINFGSSKLL
LNCFYKYNKKSGINSDQLENKFNEFQNGLNIICSPLQPIRGSSKRSFEFIRNSIINFLMYSLYAKL
FGIPRSVKALMKSNKDENKLKLEEKLKKKKSSFNKTVKEFEKMIGRKLSDNESKILNDESKKFF
EIIKSNNKYIPSEEYLKLLKDISEEIYNSNIDFKPYKYSILIRKPLSKFKSKKLYNLKPTDYKYYLQ
LSYEPFSKQLIATKTILGIDRGLKHLLAVSVFDPSQNKFVYNKLIKNPVFKWKKRYHDLKRSIR
NRERRIRALTGVHIHENQLIKKLKSMKNKINVLYHNVSKNIVDLAKKYESTIVLERLENLKQHG
RSKGKRYKKLNYVLSNFDYKKIESLISYKAKKEGVPVSNINPKYTSKTCAKCLLEVNQLSELK
NEYNRDSKNSKIGICNIHGQIDADLNAARVIALCYSKNLNEPHFK (SEQ ID NO: 134)
VINLFGYKFALYPNKTQEELLNKHLGECGWLYNKAIEQNEYYKADSNIEEAQKKFELLPDKNS
DEAKVLRGNISKDNYVYRTLVKKKKSEINVQIRKAVVLRPAETIRNLAKVKKKGLSVGRLKFI
PIREWDVLPFKQSDQIRLEENYLILEPYGRLKFKMHRPLLGKPKTFCIKRTATDRWTISFSTEYD
DSNMRKNDGGQVGIDVGLKTHLRLSNENPDEDPRYPNPKIWKRYDRRLTILQRRISKSKKLGK
NRTRLRLRLSRLWEKIRNSRADLIQNETYEILSENKLIAIEDLNVKGMQEKKDKKGRKGRTRA
QEKGLHRSISDAAFSEFRRVLEYKAKRFGSEVKPVSAIDSSKECHNCGNKKGMPLESRIYECPK
CGLKIDRDLNSAKVILARATGVRPGSNARADTKISATAGASVQTEGTVSEDFRQQMETSDQKP
MQGEGSKEPPMNPEHKSSGRGSKHVNIGCKNKVGLYNEDENSRSTEKQIMDENRSTTEDMVE
IGALHSPVLTT (SEQ ID NO: 135)
MIASIDYEAVSQALIVFEFKAKGKDSQYQAIDEAIRSYRFIRNSCLRYWMDNKKVGKYDLNKY
CKVLAKQYPFANKLNSQARQSAAECSWSAISRFYDNCKRKVSGKKGFPKFKKHARSVEYKTS
GWKLSENRKAITFTDKNGIGKLKLKGTYDLHFSQLEDMKRVRLVRRADGYYVQFCISVDVKV
ETEPTGKAIGLDVGIKYFLADSSGNTIENPQFYRKAEKKLNRANRRKSKKYIRGVKPQSKNYH
KARCRYARKHLRVSRQRKEYCKRVAYCVIHSNDVVAYEDLNVKGMVKNRHLAKSISDVAW
STFRHWLEYFAIKYGKLTIPVAPHNTSQNCSNCDKKVPKSLSTRTHICHHCGYSEDRDVNAAK
NILKKALSTVGQTGSLKLGEIEPLLVLEQSCTRKFDL (SEQ ID NO: 136)
LAEENTLHLTLAMSLPLNDLPENRTRSELWRRQWLPQKKLSLLLGVNQSVRKAAADCLRWFE
PYQELLWWEPTDPDGKKLLDKEGRPIKRTAGHMRVLRKLEEIAPFRGYQLGSAVKNGLRHKV
ADLLLSYAKRKLDPQFTDKTSYPSIGDQFPIVWTGAFVCYEQSITGQLYLYLPLFPRGSHQEDIT
NNYDPDRGPALQVFGEKEIARLSRSTSGLLLPLQFDKWGEATFIRGENNPPTWKATHRRSDKK
WLSEVLLREKDFQPKRVELLVRNGRIFVNVACEIPTKPLLEVENFMGVSFGLEHLVTVVVINRD
GNVVHQRQEPARRYEKTYFARLERLRRRGGPFSQELETFHYRQVAQIVEEALRFKSVPAVEQV
GNIPKGRYNPRLNLRLSYWPFGKLADLTSYKAVKEGLPKPYSVYSATAKMLCSTCGAANKEG
DQPISLKGPTVYCGNCGTRHNTGENTALNLARRAQELFVKGVVAR (SEQ ID NO: 137)
MSQSLLKWHDMAGRDKDASRSLQKSAVEGVLLHLTASHRVALEMLEKSVSQTVAVTMEAA
QQRLVIVLEDDPTKATSRKRVISADLQFTREEFGSLPNWAQKLASTCPEIATKYADKHINSIRIA
WGVAKESTNGDAVEQKLQWQIRLLDVTMFLQQLVLQLADKALLEQIPSSIRGGIGQEVAQQV
TSHIQLLDSGTVLKAELPTISDRNSELARKQWEDAIQTVCTYALPFSRERARILDPGKYAAEDP
RGDRLINIDPMWARVLKGPTVKSLPLLFVSGSSIRIVKLTLPRKHAAGHKHTFTATYLVLPVSR
EWINSLPGTVQEKVQWWKKPDVLATQELLVGKGALKKSANTLVIPISAGKKRFFNHILPALQR
GFPLQWQRIVGRSYRRPATHRKWFAQLTIGYTNPSSLPEMALGIHFGMKDILWWALADKQGN
ILKDGSIPGNSILDFSLQEKGKIERQQKAGKNVAGKKYGKSLLNATYRVVNGVLEFSKGISAEH
ASQPIGLGLETIRFVDKASGSSPVNARHSNWNYGQLSGIFANKAGPAGFSVTEITLKKAQRDLS
DAEQARVLAIEATKRFASRIKRLATKRKDDTLFV (SEQ ID NO: 138)
VEPVEKERFYYRTYTFRLDGQPRTQNLTTQSGWGLLTKAVLDNTKHYWEIVHHARIANQPIVF
ENPVIDEQGNPKLNKLGQPRFWKRPISDIVNQLRALFENQNPYQLGSSLIQGTYWDVAENLAS
WYALNKEYLAGTATWGEPSFPEPHPLTEINQWMPLTFSSGKVVRLLKNASGRYFIGLPILGEN
NPCYRMRTIEKLIPCDGKGRVTSGSLILFPLVGIYAQQHRRMTDICESIRTEKGKLAWAQVSIDY
VREVDKRRRMRRTRKSQGWIQGPWQEVFILRLVLAHKAPKLYKPRCFAGISLGPKTLASCVIL
DQDERVVEKQQWSGSELLSLIHQGEERLRSLREQSKPTWNAAYRKQLKSLINTQVFTIVTFLRE
RGAAVRLESIARVRKSTPAPPVNFLLSHWAYRQITERLKDLAIRNGMPLTHSNGSYGVRFTCS
QCGATNQGIKDPTKYKVDIESETFLCSICSHREIAAVNTATNLAKQLLDE (SEQ ID NO: 139)
MNDTETSETLTSHRTVCAHLHVVGETGSLPRLVEAALAELITLNGRATQALLSLAKNGLVLRR
DKEENLIAAELTLPCRKNKYADVAAKAGEPILATRINNKGKLVTKKWYGEGNSYHIVRFTPET
GMFTVRVFDRYAFDEELLHLHSEVVFGSDLPKGIKAKTDSLPANFLQAVFTSFLELPFQGFPDI
VVKPAMKQAAEQLLSYVQLEAGENQQAEYPDTNERDPELRLVEWQKSLHELSVRTEPFEFVR
ARDIDYYAETDRRGNRFVNITPEWTKFAESPFARRLPLKIPPEFCILLRRKTEGHAKIPNRIYLGL
QIFDGVTPDSTLGVLATAEDGKLFWWHDHLDEFSNLEGKPEPKLKNKPQLLMVSLEYDREQR
FEESVGGDRKICLVTLKETRNFRRGWNGRILGIHFQHNPVITWALMDHDAEVLEKGFIEGNAF
LGKALDKQALNEYLQKGGKWVGDRSFGNKLKGITHTLASLIVRLAREKDAWIALEEISWVQK
QSADSVANHEIVEQPHHSLTR (SEQ ID NO: 140)
MNDTETSETLTSHRTVCAHLHVVGETGSLPRLVEAALAELITLNGRATQALLSLAKNGLVLRR
DKEENLIAAELTLPCRKNKYADVAAKAGEPILATRINNKGKLVTKKWYGEGNSYHIVRFTPET
GMFTVRVFDRYAFDEELLHLHSEVVFGSDLPKGIKAKTDSLPANFLQAVFTSFLELPFQGFPDI
VVKPAMKQAAEQLLSYVQLEAGENQQAEYPDTNERDPELRLVEWQKSLHELSVRTEPFEFVR
ARDIDYYAETDRRGNRFVNITPEWTKFAESPFARRLPLKIPPEFCILLRRKTEGHAKIPNRIYLGL
QIFDGVTPDSTLGVLATAEDGKLFWWHDHLDEFSNLEGKPEPKLKNKPQLLMVSLEYDREQR
FEESVGGDRKICLVTLKETRNFRRGRHGHTRTDRLPAGNTLWRADFATSAEVAAPKWNGRIL
GIHFQHNPVITWALMDHDAEVLEKGFIEGNAFLGKALDKQALNEYLQKGGKWVGDRSFGNK
LKGITHTLASLIVRLAREKDAWIALEEISWVQKQSADSVANRRFSMWNYSRLATLIEWLGTDI
ATRDCGTAAPLAHKVSDYLTHFTCPECGACRKAGQKKEIADTVRAGDILTCRKCGFSGPIPDN
FIAEFVAKKALERMLKKKPV (SEQ ID NO: 141)
MAKRNFGEKSEALYRAVRFEVRPSKEELSILLAVSEVLRMLFNSALAERQQVFTEFIASLYAEL
KSASVPEEISEIRKKLREAYKEHSISLFDQINALTARRVEDEAFASVTRNWQEETLDALDGAYK
SFLSLRRKGDYDAHSPRSRDSGFFQKIPGRSGFKIGEGRIALSCGAGRKLSFPIPDYQQGRLAET
TKLKKFELYRDQPNLAKSGRFWISVVYELPKPEATTCQSEQVAFVALGASSIGVVSQRGEEVIA
LWRSDKHWVPKIEAVEERMKRRVKGSRGWLRLLNSGKRRMHMISSRQHVQDEREIVDYLVR
NHGSHFVVTELVVRSKEGKLADSSKPERGGSLGLNWAAQNTGSLSRLVRQLEEKVKEHGGSV
RKHKLTLTEAPPARGAENKLWMARKLRESFLKEV (SEQ ID NO: 142)
LAKNDEKELLYQSVKFEIYPDESKIRVLTRVSNILVLVWNSALGERRARFELYIAPLYEELKKFP
RKSAESNALRQKIREGYKEHIPTFFDQLKKLLTPMRKEDPALLGSVPRAYQEETLNTLNGSFVS
FMTLRRNNDMDAKPPKGRAEDRFHEISGRSGFKIDGSEFVLSTKEQKLRFPIPNYQLEKLKEAK
QIKKFTLYQSRDRRFWISIAYEIELPDQRPFNPEEVIYIAFGASSIGVISPEGEKVIDFWRPDKHW
KPKIKEVENRMRSCKKGSRAWKKRAAARRKMYAMTQRQQKLNHREIVASLLRLGFHFVVTE
YTVRSKPGKLADGSNPKRGGAPQGFNWSAQNTGSFGEFILWLKQKVKEQGGTVQTFRLVLGQ
SERPEKRGRDNKIEMVRLLREKYLESQTIVV (SEQ ID NO: 143)
MAKGKKKEGKPLYRAVRFEIFPTSDQITLFLRVSKNLQQVWNEAWQERQSCYEQFFGSIYERI
GQAKKRAQEAGFSEVWENEAKKGLNKKLRQQEISMQLVSEKESLLQELSIAFQEHGVTLYDQI
NGLTARRIIGEFALIPRNWQEETLDSLDGSFKSFLALRKNGDPDAKPPRQRVSENSFYKIPGRSG
FKVSNGQIYLSFGKIGQTLTSVIPEFQLKRLETAIKLKKFELCRDERDMAKPGRFWISVAYEIPK
PEKVPVVSKQITYLAIGASRLGVVSPKGEFCLNLPRSDYHWKPQINALQERLEGVVKGSRKWK
KRMAACTRMFAKLGHQQKQHGQYEVVKKLLRHGVHFVVTELKVRSKPGALADASKSDRKG
SPTGPNWSAQNTGNIARLIQKLTDKASEHGGTVIKRNPPLLSLEERQLPDAQRKIFIAKKLREEF
LADQK (SEQ ID NO: 144)
MAKREKKDDVVLRGTKMRIYPTDRQVTLMDMWRRRCISLWNLLLNLETAAYGAKNTRSKL
GWRSIWARVVEENHAKALIVYQHGKCKKDGSFVLKRDGTVKHPPRERFPGDRKILLGLFDAL
RHTLDKGAKCKCNVNQPYALTRAWLDETGHGARTADIIAWLKDFKGECDCTAISTAAKYCPA
PPTAELLTKIKRAAPADDLPVDQAILLDLFGALRGGLKQKECDHTHARTVAYFEKHELAGRAE
DILAWLIAHGGTCDCKIVEEAANHCPGPRLFIWEHELAMIMARLKAEPRTEWIGDLPSHAAQT
VVKDLVKALQTMLKERAKAAAGDESARKTGFPKFKKQAYAAGSVYFPNTTMFFDVAAGRV
QLPNGCGSMRCEIPRQLVAELLERNLKPGLVIGAQLGLLGGRIWRQGDRWYLSCQWERPQPT
LLPKTGRTAGVKIAASIVFTTYDNRGQTKEYPMPPADKKLTAVHLVAGKQNSRALEAQKEKE
KKLKARKERLRLGKLEKGHDPNALKPLKRPRVRRSKLFYKSAARLAACEAIERDRRDGFLHR
VTNEIVHKFDAVSVQKMSVAPMMRRQKQKEKQIESKKNEAKKEDNGAAKKPRNLKPVRKLL
RHVAMARGRQFLEYKYNDLRGPGSVLIADRLEPEVQECSRCGTKNPQMKDGRRLLRCIGVLP
DGTDCDAVLPRNRNAARNAEKRLRKHREAHNA (SEQ ID NO: 145)
MNEVLPIPAVGEDAADTIMRGSKMRIYPSVRQAATMDLWRRRCIQLWNLLLELEQAAYSGEN
RRTQIGWRSIWATVVEDSHAEAVRVAREGKKRKDGTFRKAPSGKEIPPLDPAMLAKIQRQMN
GAVDVDPKTGEVTPAQPRLFMWEHELQKIMARLKQAPRTHWIDDLPSHAAQSVVKDLIKALQ
AMLRERKKRASGIGGRDTGFPKFKKNRYAAGSVYFANTQLRFEAKRGKAGDPDAVRGEFAR
VKLPNGVGWMECRMPRHINAAHAYAQATLMGGRIWRQGENWYLSCQWKMPKPAPLPRAG
RTAAIKIAAAIPITTVDNRGQTREYAMPPIDRERIAAHAAAGRAQSRALEARKRRAKKREAYA
KKRHAKKLERGIAAKPPGRARIKLSPGFYAAAAKLAKLEAEDANAREAWLHEITTQIVRNFDV
IAVPRMEVAKLMKKPEPPEEKEEQVKAPWQGKRRSLKAARVMMRRTAMALIQTTLKYKAVD
LRGPQAYEEIAPLDVTAAACSGCGVLKPEWKMARAKGREIMRCQEPLPGGKTCNTVLTYTRN
SARVIGRELAVRLAERQKA (SEQ ID NO: 146)
MTTQKTYNFCFYDQRFFELSKEAGEVYSRSLEEFWKIYDETGVWLSKFDLQKHMRNKLERKL
LHSDSFLGAMQQVHANLASWKQAKKVVPDACPPRKPKFLQAILFKKSQIKYKNGFLRLTLGT
EKEFLYLKWDINIPLPIYGSVTYSKTRGWKINLCLETEVEQKNLSENKYLSIDLGVKRVATIFDG
ENTITLSGKKFMGLMHYRNKLNGKTQSRLSHKKKGSNNYKKIQRAKRKTTDRLLNIQKEMLH
KYSSFIVNYAIRNDIGNIIIGDNSSTHDSPNMRGKTNQKISQNPEQKLKNYIKYKFESISGRVDIV
PEPYTSRKCPHCKNIKKSSPKGRTYKCKKCGFIFDRDGVGAINIYNENVSFGQIISPGRIRSLTEPI
GMKFHNEIYFKSYVAA (SEQ ID NO: 147)
MSVRSFQARVECDKQTMEHLWRTHKVFNERLPEIIKILFKMKRGECGQNDKQKSLYKSISQSIL
EANAQNADYLLNSVSIKGWKPGTAKKYRNASFTWADDAAKLSSQGIHVYDKKQVLGDLPGM
MSQMVCRQSVEAISGHIELTKKWEKEHNEWLKEKEKWESEDEHKKYLDLREKFEQFEQSIGG
KITKRRGRWHLYLKWLSDNPDFAAWRGNKAVINPLSEKAQIRINKAKPNKKNSVERDEFFKA
NPEMKALDNLHGYYERNFVRRRKTKKNPDGFDHKPTFTLPHPTIHPRWFVFNKPKTNPEGYR
KLILPKKAGDLGSLEMRLLTGEKNKGNYPDDWISVKFKADPRLSLIRPVKGRRVVRKGKEQG
QTKETDSYEFFDKHLKKWRPAKLSGVKLIFPDKTPKAAYLYFTCDIPDEPLTETAKKIQWLETG
DVTKKGKKRKKKVLPHGLVSCAVDLSMRRGTTGFATLCRYENGKIHILRSRNLWVGYKEGK
GCHPYRWTEGPDLGHIAKHKREIRILRSKRGKPVKGEESHIDLQKHIDYMGEDRFKKAARTIV
NFALNTENAASKNGFYPRADVLLLENLEGLIPDAEKERGINRALAGWNRRHLVERVIEMAKD
AGFKRRVFEIPPYGTSQVCSKCGALGRRYSIIRENNRREIRFGYVEKLFACPNCGYCANADHNA
SVNLNRRFLIEDSFKSYYDWKRLSEKKQKEEIETIESKLMDKLCAMHKISRGSISK (SEQ ID
NO: 148)
MHLWRTHCVFNQRLPALLKRLFAMRRGEVGGNEAQRQVYQRVAQFVLARDAKDSVDLLNA
VSLRKRSANSAFKKKATISCNGQAREVTGEEVFAEAVALASKGVFAYDKDDMRAGLPDSLFQ
PLTRDAVACMRSHEELVATWKKEYREWRDRKSEWEAEPEHALYLNLRPKFEEGEAARGGRF
RKRAERDHAYLDWLEANPQLAAWRRKAPPAVVPIDEAGKRRIARAKAWKQASVRAEEFWK
RNPELHALHKIHVQYLREFVRPRRTRRNKRREGFKQRPTFTMPDPVRHPRWCLFNAPQTSPQG
YRLLRLPQSRRTVGSVELRLLTGPSDGAGFPDAWVNVRFKADPRLAQLRPVKVPRTVTRGKN
KGAKVEADGFRYYDDQLLIERDAQVSGVKLLFRDIRMAPFADKPIEDRLLSATPYLVFAVEIK
DEARTERAKAIRFDETSELTKSGKKRKTLPAGLVSVAVDLDTRGVGFLTRAVIGVPEIQQTHH
GVRLLQSRYVAVGQVEARASGEAEWSPGPDLAHIARHKREIRRLRQLRGKPVKGERSHVRLQ
AHIDRMGEDRFKKAARKIVNEALRGSNPAAGDPYTRADVLLYESLETLLPDAERERGINRALL
RWNRAKLIEHLKRMCDDAGIRHFPVSPFGTSQVCSKCGALGRRYSLARENGRAVIRFGWVER
LFACPNPECPGRRPDRPDRPFTCNSDHNASVNLHRVFALGDQAVAAFRALAPRDSPARTLAVK
RVEDTLRPQLMRVHKLADAGVDSPF (SEQ ID NO: 149)
MATLVYRYGVRAHGSARQQDAVVSDPAMLEQLRLGHELRNALVGVQHRYEDGKRAVWSGF
ASVAAADHRVTTGETAVAELEKQARAEHSADRTAATRQGTAESLKAARAAVKQARADRKA
AMAAVAEQAKPKIQALGDDRDAEIKDLYRRFCQDGVLLPRCGRCAGDLRSDGDCTDCGAAH
EPRKLYWATYNAIREDHQTAVKLVEAKRKAGQPARLRFRRWTGDGTLTVQLQRMHGPACRC
VTCAEKLTRRARKTDPQAPAVAADPAYPPTDPPRDPALLASGQGKWRNVLQLGTWIPPGEWS
AMSRAERRRVGRSHIGWQLGGGRQLTLPVQLHRQMPADADVAMAQLTRVRVGGRHRMSVA
LTAKLPDPPQVQGLPPVALHLGWRQRPDGSLRVATWACPQPLDLPPAVADVVVSHGGRWGE
VIMPARWLADAEVPPRLLGRRDKAMEPVLEALADWLEAHTEACTARMTPALVRRWRSQGRL
AGLTNRWRGQPPTGSAEILTYLEAWRIQDKLLWERESHLRRRLAARRDDAWRRVASWLARH
AGVLVVDDADIAELRRRDDPADTDPTMPASAAQAARARAALAAPGRLRHLATITATRDGLGV
HTVASAGLTRLHRKCGHQAQPDPRYAASAVVTCPGCGNGYDQDYNAAMLMLDRQQQP
(SEQ ID NO: 150)
MSRVELHRAYKFRLYPTPAQVAELAEWERQLRRLYNLAHSQRLAAMQRHVRPKSPGVLKSE
CLSCGAVAVAEIGTDGKAKKTVKHAVGCSVLECRSCGGSPDAEGRTAHTAACSFVDYYRQG
REMTQLLEEDDQLARVVCSARQETLRDLEKAWQRWHKMPGFGKPHFKKRIDSCRIYFSTPKS
WAVDLGYLSFTGVASSVGRIKIRQDRVWPGDAKFSSCHVVRDVDEWYAVFPLTFTKEIEKPK
GGAVGINRGAVHAIADSTGRVVDSPKFYARSLGVIRHRARLLDRKVPFGRAVKPSPTKYHGLP
KADIDAAAARVNASPGRLVYEARARGSIAAAEAHLAALVLPAPRQTSQLPSEGRNRERARRFL
ALAHQRVRRQREWFLHNESAHYAQSYTKIAIEDWSTKEMTSSEPRDAEEMKRVTRARNRSIL
DVGWYELGRQIAYKSEATGAEFAKVDPGLRETETHVPEAIVRERDVDVSGMLRGEAGISGTCS
RCGGLLRASASGHADAECEVCLHVEVGDVNAAVNVLKRAMFPGAAPPSKEKAKVTIGIKGRK
KKRAA (SEQ ID NO: 151)
MSRVELHRAYKFRLYPTPVQVAELSEWERQLRRLYNLGHEQRLLTLTRHLRPKSPGVLKGEC
LSCDSTQVQEVGADGRPKTTVRHAEQCPTLACRSCGALRDAEGRTAHTVACAFVDYYRQGR
EMTELLAADDQLARVVCSARQEVLRDLDKAWQRWRKMPGFGKPRFKRRTDSCRIYFSTPKA
WKLEGGHLSFTGAATTVGAIKMRQDRNWPASVQFSSCHVVRDVDEWYAVFPLTFVAEVARP
KGGAVGINRGAVHAIADSTGRVVDSPRYYARALGVIRHRARLFDRKVPSGHAVKPSPTKYRG
LSAIEVDRVARATGFTPGRVVTEALNRGGVAYAECALAAIAVLGHGPERPLTSDGRNREKAR
KFLALAHQRVRRQREWFLHNESAHYARTYSKIAIEDWSTKEMTASEPQGEETRRVTRSRNRSI
LDVGWYELGRQLAYKTEATGAEFAQVDPGLKETETNVPKAIADARDVDVSGMLRGEAGISGT
CSKCGGLLRAPASGHADAECEICLNVEVGDVNAAVNVLKRAMFPGDAPPASGEKPKVSIGIK
GRQKKKKAA (SEQ ID NO: 152)
MEAIATGMSPERRVELGILPGSVELKRAYKFRLYPMKVQQAELSEWERQLRRLYNLAHEQRL
AALLRYRDWDFQKGACPSCRVAVPGVHTAACDHVDYFRQAREMTQLLEVDAQLSRVICCAR
QEVLRDLDKAWQRWRKKLGGRPRFKRRTDSCRIYLSTPKHWEIAGRYLRLSGLASSVGEIRIE
QDRAFPEGALLSSCSIVRDVDEWYACLPLTFTQPIERAPHRSVGLNRGVVHALADSDGRVVDS
PKFFERALATVQKRSRDLARKVSGSRNAHKARIKLAKAHQRVRRQRAAFLHQESAYYSKGFD
LVALEDMSVRKMTATAGEAPEMGRGAQRDLNRGILDVGWYELARQIDYKRLAHGGELLRVD
PGQTTPLACVTEEQPARGISSACAVCGIPLARPASGNARMRCTACGSSQVGDVNAAENVLTRA
LSSAPSGPKSPKASIKIKGRQKRLGTPANRAGEASGGDPPVRGPVEGGTLAYVVEPVSESQSDT
(SEQ ID NO: 153)
MTVRTYKYRAYPTPEQAEALTSWLRFASQLYNAALEHRKNAWGRHDAHGRGFRFWDGDAA
PRKKSDPPGRWVYRGGGGAHISKNDQGKLLTEFRREHAELLPPGMPALVQHEVLARLERSMA
AFFQRATKGQKAGYPRWRSEHRYDSLTFGLTSPSKERFDPETGESLGRGKTVGAGTYHNGDL
RLTGLGELRILEHRRIPMGAIPKSVIVRRSGKRWFVSIAMEMPSVEPAASGRPAVGLDMGVVT
WGTAFTADTSAAAALVADLRRMATDPSDCRRLEELEREAAQLSEVLAHCRARGLDPARPRRC
PKELTKLYRRSLHRLGELDRACARIRRRLQAAHDIAEPVPDEAGSAVLIEGSNAGMRHARRVA
RTQRRVARRTRAGHAHSNRRKKAVQAYARAKERERSARGDHRHKVSRALVRQFEEISVEAL
DIKQLTVAPEHNPDPQPDLPAHVQRRRNRGELDAAWGAFFAALDYKAADAGGRVARKPAPH
TTQECARCGTLVPKPISLRVHRCPACGYTAPRTVNSARNVLQRPLEEPGRAGPSGANGRGVPH
AVA (SEQ ID NO: 154)
MNCRYRYRIYPTPGQRQSLARLFGCVRVVWNDALFLCRQSEKLPKNSELQKLCITQAKKTEA
RGWLGQVSAIPLQQSVADLGVAFKNFFQSRSGKRKGKKVNPPRVKRRNNRQGARFTRGGFK
VKTSKVYLARIGDIKIKWSRPLPSEPSSVTVIKDCAGQYFLSFVVEVKPEIKPPKNPSIGIDLGLK
TFASCSNGEKIDSPDYSRLYRKLKRCQRRLAKRQRGSKRRERMRVKVAKLNAQIRDKRKDFL
HKLSTKVVNENQVIALEDLNVGGMLKNRKLSRAISQAGWYEFRSLCEGKAEKHNRDFRVISR
WEPTSQVCSECGYRWGKIDLSVRSIVCINCGVEHDRDDNASVNIEQAGLKVGVGHTHDSKRT
GSACKTSNGAVCVEPSTHREYVQLTLFDW (SEQ ID NO: 155)
MKSRWTFRCYPTPEQEQHLARTFGCVRFVWNWALRARTDAFRAGERIGYPATDKALTLLKQ
QPETVWLNEVSSVCLQQALRDLQVAFSNFFDKRAAHPSFKRKEARQSANYTERGFSFDHERRI
LKLAKIGAIKVKWSRKAIPHPSSIRLIRTASGKYFVSLVVETQPAPMPETGESVGVDFGVARLA
TLSNGERISNPKHGAKWQRRLAFYQKRLARATKGSKRRMRIKRHVARIHEKIGNSRSDTLHKL
STDLVTRFDLICVEDLNLRGMVKNHSLARSLHDASIGSAIRMIEEKAERYGKNVVKIDRWFPSS
KTCSDCGHIVEQLPLNVREWTCPECGTTHDRDANAAANILAVGQTVSAHGGTVRRSRAKASE
RKSQRSANRQGVNRA (SEQ ID NO: 156)
M08
MKKIDNFVGCYPVSKTLRFKAIPIGKTQENIEKKRLVEEDEVRAKDYKAVKKLIDRYHREFIEG
VLDNVKLDGLEEYYMLFNKSDREESDNKKIEIMEERFRRVISKSFKNNEEYKKIFSKKIIEEILPN
YIKDEEEKELVKGFKGFYTAFVGYAQNRENMYSDEKKSTAISYRIVNENMPRFITNIKVFEKAK
SILDVDKINEINEYILNNDYYVDDFFNIDFFNYVLNQKGIDIYNAIIGGIVTGDGRKIQGLNECIN
LYNQENKKIRLPQFKPLYKQILSESESMSFYIDEIESDDMLIDMLKESLQIDSTINNAIDDLKVLF
NNIFDYDLSGIFINNGLPITTISNDVYGQWSTISDGWNERYDVLSNAKDKESEKYFEKRRKEYK
KVKSFSISDLQELGGKDLSICKKINEIISEMIDDYKSKIEEIQYLFDIKELEKPLVTDLNKIELIKNS
LDGLKRIERYVIPFLGTGKEQNRDEVFYGYFIKCIDAIKEIDGVYNKTRNYLTKKPYSKDKFKL
YFENPQLMGGWDRNKESDYRSTLLRKNGKYYVAIIDKSSSNCMMNIEEDENDNYEKINYKLL
PGPNKMLPKVFFSKKNREYFAPSKEIERIYSTGTFKKDTNFVKKDCENLITFYKDSLDRHEDWS
KSFDFSFKESSAYRDISEFYRDVEKQGYRVSFDLLSSNAVNTLVEEGKLYLFQLYNKDFSEKSH
GIPNLHTMYFRSLFDDNNKGNIRLNGGAEMFMRRASLNKQDVTVHKANQPIKNKNLLNPKKT
TTLPYDVYKDKRFTEDQYEVHIPITMNKVPNNPYKINHMVREQLVKDDNPYVIGIDRGERNLI
YVVVVDGQGHIVEQLSLNEIINENNGISIRTDYHTLLDAKERERDESRKQWKQIENIKELKEGYI
SQVVHKICELVEKYDAVIALEDLNSGFKNSRVKVEKQVYQKFEKMLITKLNYMVDKKKDYN
KPGGVLNGYQLTTQFESFSKMGTQNGIMFYIPAWLTSKMDPTTGFVDLLKPKYKNKADAQKF
FSQFDSIRYDNQEDAFVFKVNYTKFPRTDADYNKEWEIYTNGERIRVFRNPKKNNEYDYETVN
VSERMKELFDSYDLLYDKGELKETICEMEESKFFEELIKLFRLTLQMRNSISGRTDVDYLISPVK
NSNGYFYNSNDYKKEGAKYPKDADANGAYNIARKVLWAIEQFKMADEDKLDKTKISIKNQE
WLEYAQTHCE (SEQ ID NO: 236)
CasM21
MSKLEKFTNCYSLSKTLRFKAIPVGKTQENIDNKRLLVEDEKRAEDYKGVKKLLDRYYLSFIN
DVLHSIKLKNLNNYISLFRKKTRTEKENKELENLEINLRKEIAKAFKGNEGYKSLFKKDIIETILP
EFLDDKDEIALVNSFNGFTTAFTGFFDNRENMFSEEAKSTSIAFRCINENLTRYISNMDIFEKVD
AIFDKHEVQEIKEKILNSDYDVEDFFEGEFFNFVLTQEGIDVYNAIIGGFVTESGEKIKGLNEYIN
LYNQKTKQKLPKFKPLYKQVLSDRESLSFYGEGYTSDEEVLEVFRNTLNKNSEIFSSIKKLEKLF
KNFDEYSSAGIFVKNGPAISTISKDIFGEWNVIRDKWNAEYDDIHLKKKAVVTEKYEDDRRKSF
KKIGSFSLEQLQEYADADLSVVEKLKEIIIQKVDEIYKVYGSSEKLFDADFVLEKSLKKNDAVV
AIMKDLLDSVKSFENYIKAFFGEGKETNRDESFYGDFVLAYDILLKVDHIYDAIRNYVTQKPYS
KDKFKLYFQNPQFMGGWDKDKETDYRATILRYGSKYYLAIMDKKYAKCLQKIDKDDVNGN
YEKINYKLLPGPNKMLPKVFFSKKWMAYYNPSEDIQKIYKNGTFKKGDMFNLNDCHKLIDFF
KDSISRYPKWSNAYDFNFSETEKYKDIAGFYREVEEQGYKVSFESASKKEVDKLVEEGKLYMF
QIYNKDFSDKSHGTPNLHTMYFKLLFDENNHGQIRLSGGAELFMRRASLKKEELVVHPANSPI
ANKNPDNPKKTTTLSYDVYKDKRFSEDQYELHIPIAINKCPKNIFKINTEVRVLLKHDDNPYVI
GIDRGERNLLYIVVVDGKGNIVEQYSLNEIINNFNGIRIKTDYHSLLDKKEKERFEARQNWTSIE
NIKELKAGYISQVVHKICELVEKYDAVIALEDLNSGFKNSRVKVEKQVYQKFEKMLIDKLNYM
VDKKSNPCATGGALKGYQITNKFESFKSMSTQNGFIFYIPAWLTSKIDPSTGFVNLLKTKYTSIA
DSKKFISSFDRIMYVPEEDLFEFALDYKNFSRTDADYIKKWKLYSYGNRIRIFRNPKKNNVFDW
EEVCLTSAYKELFNKYGINYQQGDIRALLCEQSDKAFYSSFMALMSLMLQMRNSITGRTDVDF
LISPVKNSDGIFYDSRNYEAQENAILPKNADANGAYNIARKVLWAIGQFKKAEDEKLDKVKIAI
SNKEWLEYAQTSVKH (SEQ ID NO: 237)
CasM26
MKVTKVGGISHKKYTSEGRLVKSESEENRTDERLSALLNMRLDMYIKNPSSTETKENQKRIGK
LKKFFSNKMVYLKDNTLSLKNGKKENIDREYSETDILESDVRDKKNFAVLKKIYLNENVNSEE
LEVFRNDIKKKLNKINSLKYSFEKNKANYQKINENNIEKVEGKSKRNIIYDYYRESAKRDAYVS
NVKEAFDKLYKEEDIAKLVLEIENLTKLEKYKIREFYHEIIGRKNDKENFAKIIYEEIQNVNNMK
ELIEKVPDMSELKKSQVFYKYYLDKEELNDKNIKYAFCHFVEIEMSQLLKNYVYKRLSNISND
KIKRIFEYQNLKKLIENKLLNKLDTYVRNCGKYNYYLQDGEIATSDFIARNRQNEAFLRNIIGVS
SVAYFSLRNILETENENDITGRMRGKTVKNNKGEEKYVSGEVDKIYNENKKNEVKENLKMFY
SYDFNMDNKNEIEDFFANIDEAISSIRHGIVHFNLELEGKDIFAFKNIAPSEISKKMFQNEINEKK
LKLKIFRQLNSANVFRYLEKYKILNYLKRTRFEFVNKNIPFVPSFTKLYSRIDDLKNSLGIYWKT
PKTNDDNKTKEIIDAQIYLLKNIYYGEFLNYFMSNNGNFFEISKEIIELNKNDKRNLKTGFYKLQ
KFEDIQEKIPKEYLANIQSLYMINAGNQDEEEKDTYIDFIQKIFLKGFMTYLANNGRLSLIYIGSD
EETNTSLAEKKQEFDKFLKKYEQNNNIKIPYEINEFLREIKLGNILKYTERLNMFYLILKLLNHK
ELTNLKGSLEKYQSANKEEAFSDQLELINLLNLDNNRVTEDFELEADEIGKFLDFNGNKVKDN
KELKKFDTNKIYFDGENIIKHRAFYNIKKYGMLNLLEKIADKAGYKISIEELKKYSNKKNEIEK
NHKMQENLHRKYARPRKDEKFTDEDYESYKQAIENIEEYTHLKNKVEFNELNLLQGLLLRILH
RLVGYTSIWERDLRFRLKGEFPENQYIEEIFNFENKKNVKYKGGQIVEKYIKFYKELHQNDEVK
INKYSSANIKVLKQEKKDLYIRNYIAHFNYIPHAEISLLEVLENLRKLLSYDRKLKNAVMKSVV
DILKEYGFVATFKIGADKKIGIQTLESEKIVHLKNLKKKKLMTDRNSEELCKLVKIMFEYKMEE
KKSEN (SEQ ID NO: 238)
Cas12d.3
MKAKKSFYNQKRKFGKRGYRLHDERIAYSGGIGSMRSIKYELKDSYGIAGLRNRIADATISDN
KWLYGNINLNDYLEWRSSKTDKQIEDGDRESSLLGFWLEALRLGFVFSKQSHAPNDFNETALQ
DLFETLDDDLKHVLDRKKWCDFIKIGTPKTNDQGRLKKQIKNLLKGNKREEIEKTLNESDDEL
KEKINRIADVFAKNKSDKYTIFKLDKPNTEKYPRINDVQVAFFCHPDFEEITERDRTKTLDLIIN
RFNKRYEITENKKDDKTSNRMALYSLNQGYIPRVLNDLFLFVKDNEDDFSQFLSDLENFFSFSN
EQIKIIKERLKKLKKYAEPIPGKPQLADKWDDYASDFGGKLESWYSNRIEKLKKIPESVSDLRN
NLEKIRNVLKKQNNASKILELSQKIIEYIRDYGVSFEKPEIIKFSWINKTKDGQKKVFYVAKMA
DREFIEKLDLWMADLRSQLNEYNQDNKVSFKKKGKKIEELGVLDFALNKAKKNKSTKNENG
WQQKLSESIQSAPLFFGEGNRVRNEEVYNLKDLLFSEIKNVENILMSSEAEDLKNIKIEYKEDG
AKKGNYVLNVLARFYARFNEDGYGGWNKVKTVLENIAREAGTDFSKYGNNNNRNAGRFYL
NGRERQVFTLIKFEKSITVEKILELVKLPSLLDEAYRDLVNENKNHKLRDVIQLSKTIMALVLSH
SDKEKQIGGNYIHSKLSGYNALISKRDFISRYSVQTTNGTQCKLAIGKGKSKKGNEIDRYFYAF
QFFKNDDSKINLKVIKNNSHKNIDFNDNENKINALQVYSSNYQIQFLDWFFEKHQGKKTSLEV
GGSFTIAEKSLTIDWSGSNPRVGFKRSDTEEKRVFVSQPFTLIPDDEDKERRKERMIKTKNRFIGI
DIGEYGLAWSLIEVDNGDKNNRGIRQLESGFITDNQQQVLKKNVKSWRQNQIRQTFTSPDTKI
ARLRESLIGSYKNQLESLMVAKKANLSFEYEVSGFEVGGKRVAKIYDSIKRGSVRKKDNNSQN
DQSWGKKGINEWSFETTAAGTSQFCTHCKRWSSLAIVDIEEYELKDYNDNLFKVKINDGEVRL
LGKKGWRSGEKIKGKELFGPVKDAMRPNVDGLGMKIVKRKYLKLDLRDWVSRYGNMAIFIC
PYVDCHHISHADKQAAFNIAV (SEQ ID NO: 239)
CasM.21526 (CasFire)
MLKSYDYFTKLYSLQKTLRFELKPIGKTLEHIKNSGIIESDETLEEQYAIVKNIIDKLHRKHIDEA
LSLVDFTKHLDTLKTFQELYLKRGKTDKEKEELEKLSADLRKLIVSYLKGNVKEKTQHNLNPI
KERFEILFGKELFTNEEFFLLAENEKEKKAIQAFKGFTTYFKGFQENRKNMYSEEGNSTSIAYRII
NENLPLFIENIARFQKVMSTIEKTTIKKLEQNLKTELKKHNLPGIFTIEYFNNVLTQEGISRYNTII
GGKTTHEGVKIQGLNEIINLYNQQSKDVKLPILKPLHKQILSEEYSTSFKIKAFENDNEVLKAID
TFWNEHIEKSIHPVTGNKFNILSKIENLCDQLQKYKDKDLEKLFIERKNLSTVSHQVYGQWNIIR
DALRMHLEMNNKNIKEKDIDKYLDNDAFSWKEIKDSIKIYKEHVEDAKELNENGIIKYFSAMSI
NEEDDEKEYSISLIKNINEKYNNVKSILQEDRTGKSDLHQDKEKVGIIKEFLDSLKQLQWFLRLL
YVTVPLDEKDYEFYNELEVYYEALLPLNSLYNKVRNYMTRKPYSVEKFKLNFNSPTLLDGWD
KNKETANLSIILRKNGKYYLGIMNKENNTIFEYYPGTKSNDYYEKMIYKLLPGPNKMLPKVFFS
KKGLEYYNPPKEILNIYEKGEFKKDKSGNFKKESLHTLIDFYKEAIAKNEDWEVFNFKFKNTKE
YEDISQFYRDVEEQGYLITFEKVDANYVDKLVKEGKLYLFQIYNKDFSENKKSKGNPNLHTIY
WKGLYDSENLKNVVYKLNGEAEVFYRKKSIDYPEEIYNHGHHKEELLGKFNYPIIKDRRYTQD
KFLFHVPITMNFISKEEKRVNQLACEYLSATKEDVHIIGIDRGERHLLYLSLIDKEGNIKKQLSL
NTIKNENYDKEIDYRVKLDEKEKKRDEARKNWDVIENIKELKEGYMSQVIHIIAKMMVEEKAI
LIMEDLNIGFKRGRFKVEKQVYQKFEKMLIDKLNYLVFKNKNPLEPGGSLNAYQLTSKFDSFK
KLGKQSGFIFYVPSAYTSKIDPTTGFYNFIQVDVPNLEKGKEFFSKFEKIIYNTKEDYFEFHCKY
GKFVSEPKNKDNDRKTKESLTYYNAIKDTVWVVCSTNHERYKIVRNKAGYYESHPVDVTKNL
KDIFSQANINYNEGKDIKPIIIESNNAKLLKSIAEQLKLILAMRYNNGKHGDDEKDYILSPVKNK
QGKFFCTLDGNQTLPINADANGAYNIALKGLLLIEKIKKQQGKIKDLYISNLEWFMFMMSR
(SEQ ID NO: 240)
CasM.279793
MTKQNKSVTQNTKRKNFGEFTNLYSLSKTLRFELKPVKATKTILEVERKDRENFKKDRKIAKN
YQKLKGILNELHQEFIQDVMREFSFQEKEIKEFEERYLEALNFKEKDNYKKRTQLKNAYEKVA
KKLAGKIATAFGKYNQEKYGVKFTKKNLTGENVFDILEGKYKGDKKILGIIHTFKFKPTKEEK
KQGKEAVNFSTYLGGFNQNRENFYKGEMKAGQFATRTIENLIQFLKNKKLFIDKYKDNYQKIG
FSQEQVEIFNLNYFNNLFLQEGLDVYNGILGAKKGEKNTENDGLNQKINLFKQKEKTRCKANG
EKFNKSDYPIFKELYKQIGSIKKDNDVYVEIKSDEELVNVLQSLPEKTANTLREVQKFYENFFD
KIFNDEFDLDKIYLPKSVGTHFSHLAFSDWSKLAFVFNKRWRNEKVKIKEGEDVNVQSRSLAD
IKKRMEEILEMDGGVSFGKTYCQKVGLEKEARTIEDVWSGFWKIIQYHINSQFIGGEKEVFDKE
KKDDKTEKIQTIDDLQEEYLQATEMYRERMVESEEGLNDGEEKEIKTKLKNYLDRIKDIERIAR
YFDLRKHFDDIDEASKDGDFYFIYQELLQDISEAKINDHYNEIRNYLTKANVVDDKFKLNFND
GQTLSGWDLNKETEKFSLIFKRKVDGGVEYYLGIINKEKNKTIFDKKKHPEIFTENSEFEKMEY
KLFPSPSKMLPKIAFTKNKEGERIKPVFLDENAGKEIAQIKKEFALFQDAKKEDKNKWSDEFDR
KKLNKLIDYYKLVLEKHPEKYMQTFNFVFKSSAKYKNLGEFNDDVARQNYVTKFVSVDKDYI
DQKVESGELYLFKIHNKDWNLTKAGDTKKQSKKNLHTIYFEELFSEKNIAEPVFKLSGGAEVF
FRDAIEKKKQKKKKDKKGKEILEKFRFTKNKILFHVPITINYGKPSINQGQFNQKINEFIADNSR
SVNILGIDRGEKHLLYYSLVDNNGKIIKSGSLNEINGVDYHEKLDKAEKERQEARKSWQKINQI
KNLKAGYISQVIKKIVDLAIENNAIIVLEDLNFGFKSFRQKIEKNVYQQFEKALIDKLGFVTDKE
KLNHRQAPQLSAPFESFEKMGKQTGIVFYVLATNTSKVCPQCQWKKNIFPHYSTKKSIAENLQ
KQYKMKMYWRENENRFEFEYKGDGDKEFSSIFSNVDRVRYDKRANNNQGGYVIYQIDSTTK
EKDGRNIKEKSITNLLKELLLEKFEIDNLEGELLVKLSEKSPDVSKETIKDFFGLLNSILNIRNSM
TDTEEDYIQCPACGFDTRKENKIGIKNGDDNGAYNIALRGRFLIERIKKAKKEDKKPNLTFSNN
DYFQWVREFVK (SEQ ID NO: 241)
CasM.286498
MKSIFDNFTGLYSLSKTLRFELRPVGQTLENIKNGHFLESDKKMADDYQDVKKIIDNYHKFFID
DVLKGASFDWALLEKELTDFNKNKTDDSKVEAEQKKLREQIAKTLAGDKRFKSLTASTPNDL
FNKDKDFIGWLEQSSVKEIRKDALDTFKKFSSYFKGFQENRKNVYSADDIPTAVPYRIVNDNFP
KFLQNISIFKTIQEKCPQVIADVENELASYLGKEKLADIFTVQAFNKYLCQGGKENQRGIDFYN
QVIGGIAEKEGGVNLRGINQFLNLYWQQHPDFAKENRRIKMVPLYKQILSDRSSLSFKIETIDTD
EELKTAISEYADKLESKSNDEKKSVLDVCVELFDSIKEQNLQEIYVNRKDINNISRILTGDWSW
LQSRMNLYADEVFTTKAEKTRWQKSVDGDEGENKSKGAYSLAELNRVLEYASENVAETDIRI
TDYFCHRNRFYYEKESGLFKQGEELVALSIKESCEDILSKRKAMNEAFANISESNSLRDNSEDIA
KTKTYLDSVQDLLHRIKPLKVNGLGDPSFYAVFDSIYSALSEVISIYNKTRNYITRKAESPEKYK
LNFDNPTLANGWDLNKEKDNTCVLLRKNGMYYLGIMNPKDKPKFAEKYDCGTESCYEKMIY
KLLPGPNKMLPKVFFSTKGKKQYNPPENILHGYEQGKHKKGVAFDINFCHELIDWFKSAINQH
EDWKKFGFKFSDTKSYKDISDFYREVTEQGYKLTFINIPESEISKMVSEGKLYLFQIYNMDFAP
GANGMPNMHTLYWKNLFSEENLKDVVLKLNGEAELFYRPAGIKEPVVHAKGSYLVNRITKD
GEPIPEKIHDEIYRNANGNLNNLSKEASEYKESHKVVIKQASHEIIKDRHYTEPKFLFHIPLTINF
KAPSMKPTVCTAIQNENVRRFLKNNPDVNIIGLDRGERNLIYLSLINQKGEIIKQFSFNDVEREQ
NGQTVKVNYHEKLDQREKERDAARKSWQAIGKIAELKEGYLSAVIHQLTKLMVEYNAIVVM
EDLNFGFKRGRFHVEKQVYQKFEHMLIDKLNYLVFKDRGLNEPGGALNGYQLAGQFESFQKL
GKQSGMLFYVPAAYTSKIDPKTGFVSMMNFKDLTNVHKKRDFFSKFEDIHFDEATCSFVFTFD
YKNFNGKAKEEMKQTKWAVYSREKRIVYFSKTKSSEDIMPTEKLRALFESDGIEYKSGNNIHD
SVMAVGADLKEGAKPSKEIADFWDGLLYNFKLILQMRNSNAKTGEDYIISPVMASDGTFFDSR
VEAKKGKDAKLPLDADANGAYHIALKGLSLINKINLAGEDELKKFDMKISNEDWFNFAQEKK
YAE (SEQ ID NO: 242)
CasM.1895868
MRTTTSLDAFTNRYALSKTLRFELKPIGNTQMMLEQNNVFAKDRAIREKYEKTKPWIDLLHRE
FVAESLQNAQLGNLDDYYAALQNVQKITKDTNAEDKKRWKKGFEKQEKRLRKEVVALFDKA
AHIWATQRYPQLKKKTKDFLFEEGVFEHVLFARYGSAPDTTVKIVTSNPETGEVIDEREESIFK
GWKGFTGYFDKFFETRKNFYKDNGTATAIATRAINQNLRRFAENMQKLTDIKNNYPELLAHT
DFGDFDIAHAQSLDFYARTCLLQEGIDAYNKKFVGVLKSAINEYQQKNKGVRISYPKTLDNQI
LGERERRLFDVIEDDRELHDVFRAFVDDGTVFAAEMRQLAQAFSAQNGTYDYTQIYISKKGFE
TISRKYTHDTRAWHDALADVFKAKAKKRIATTASGEKKFPAYIPVAYITQALTLVQESEDTEC
TWKERYASITENKTLEEGFFAIFADEFERLFVHMEATVQDTDYVVAEDKAKKLLSDGQITKNE
QTTQIIKEYADALLRIYQMAKYFAVEKKSMWDDAVAIDDTFYETFKEIYGNTHSTIVASYNLL
RNYLTKKPWEDVQKWKLNFENPTLLDGWDKNKEAANFGVILRDGDKFYLGIMRKGHNNIFA
NQHHSNFEGQGLQKMVYKFFPDPKKMFPKVCFSAKGMEFFAPSEEIVRIYKNAEFKSGDTFNV
ESMQKLIDFYKNALQKYDGWKIYDFKHLKDTAQYTSNIGEFYDDVAKGGYQLGWQNISKEY
VEEKNANGELYLFQIKNKDWNDGATGRKNLHTLYFEYLFSEKNAAADFVFRLNGGAEVFYRP
AAIESKTERRGNREVAAKKRYTQDKVFLHVPITLNRTAGDVKTSAFNDAVNRFLAGNPDINIM
GIDRGEKHLAYYSIIDQNGNRIVSGSFNTIGSKDYHALLTERQGAREEARKNWQRVEQIKDLK
KGYISLVVREIADLAIKHNAIIVLENLNMRFKQIRGGIEKSVYQQLEKALIEKLNFLVNKGEVDA
TKAGHLLRAYQLAAPFETFEKMGNQTGIIFYTTASYTSQVDPVTGWRPHVYLKYRNAQKTKE
DILRIFDDIVENDEKQRFEFAYRHNGVSWTVCSSVERHRWNRSNNAGKGGYDVFPVEGEGSIT
QRLQEACAQRGIDTTRNILAQIDELDESASATVSFLRDLCFYFRLICQIRNTDDGADDINAQDFL
MSPVEPFFDTRNAQEQYPQNGDENGAYNIARKGIIILQKITAWGRSQDTQRRYPDTFVSQDEW
DTFLTQHTT (SEQ ID NO: 243)
CasM.291253
MIKSYDDFTKLYSLQKTLRFELKPIGKTLEHIKKSEIIESDETLEEQYAIVKNIIDRFHRKHIDEAL
SLVDFTKHLDTFKTIQELYLKRGKTDREKKELEELSADLRKLIVSYLKGNVKQKTQHNLNPIKE
RFEILFGKELFTNEEFFTLAENKEEKKAIQAFKGFTTYFKGFQENRKNMYSEEDKSTAIAYRIIN
ENMPLFIENIARFQKVLDVIEKTKLTELKQNLKTELKGHSVSDIFRIEYFNNVLTQEGISRYNTII
GGKTTTEGVKIQGLNEIINLHNQQSKDVKLPILKPLHKQILSEEYSTSFKIKAFENDNEVLKAIDT
FWNEHIEKSIHPVTGKRFNILLKIENLCKKLEKYKDKEIEKLFIERKNLSTVSHQVYGQWNIIRD
ALRMHLEMNNKNIKEKDIDKYLDNDAFAWKEIKDSIKIYKEHVEDAKELDENGIVKYFSSMSI
NEEDDEKEYSISLIKNINEKYNNVKSILEEDRTGKSDLHQDKEKVAIIKEFLDSLKQLQWFLKLL
YVTVPLDEKDYEFYNELEVYYEALLPLNSLYNKVRNYMTRKPYSVEKFKLNFYSPTLLDGWD
KNKETANLSIILKKNGKYYLGIMNKENNTIFENFPKSKSNDYYEKMIYKLLPGPNKMLPKVFFS
KKGLEYYKPSKEILRIYEKGEFKKDKSGNFKKESLHTLIDFYKEAIAKNEDWKIFKFKFKNTRE
YEDISQFYRDVEEQGYLIIFEKVDANYVDKLVEEGELYLFQIYNKDFSENKKSKGNPNLHTIYW
ESLFDNQNLKDVVYKLNGEAEVFYRKKSIDYPEEIYNNGHHKEELNGKFNYPIIKDRRYTQDK
FLFHVPITMNFISKEEKRVNQLACEYLSTTKEDVHIIGIDRGERHLLYLSLIDKEGNIKKQLSLNT
IKNENYDKEIDYRVKLDEKEKKRDEARKNWDVIENIKELKEGYMSQVIHIIAKMMAEEKAILI
MEDLNIGFKRGRFKVEKQVYQKFEKMMIDKLNYLVFKNKEPLEPGGSLNAYQLTSKFDSFKK
LGKQSGFIFYVPSAYTSKIDPTTGFYNFIRVDVPNLEKGKEFFSKFEKIIYNTKEDYFEFHCKYG
KFVPEPKNKDNDRKTKESLTYYNAIKDTVWVVCSTHHERYKIVRNKAGYYESQPVDVTKNLR
DIFSEANINYSDGKDIKPIIIESNNAKLLKSIAEQLKLILAMRYNNGKHDDDEKDYILSPVKNKQ
GKFFCSLDGDQSLPINADANGAYNIALKGLLLIEKIKKQQGKAKDLYISNLEWFMFMMSR
(SEQ ID NO: 244)
CasM.1895869
MLKEKQFKTFGDFTNLYELSKTLRFELRPTPETKDLLDKNKIIQTDKKIAENYQEIKKYFDKLH
KKFIKEALSNTQIDFSDFCKLWEQNSKDSGKIKDLSRKLRKSIKQAFDKKGADWHKRYLEKGI
KLKKKNLDILFEERVLDILKEEFKDDVDVKLFESFKGFSTYFTNFHESRKNFYKDDGTASAIAT
RIIDENLKRFCDNIKVKKHSKKLISELNEREAKIFEADFYNRCLLQQGIDDYNQVIGDINKKINN
LRQNKIENTPTLKILYKQILGDVRRQETEQDAFIEIKNNEEVFDFLQDFIKHSDENNKYFKNLFY
KFIEGKHSLDKIFLAKRFVNTISGKWFASWEVFGAELIKKFGNKKDLPDFIPFAAVKDVLQNCN
IPANELFKEKIKNDEDKNIYDIFINLWKEEFDSNLKKVEESKKEVENMIAEDKVYSNKKEKRKN
DNGEEIEIEIQKEKIKNYADAAMNIFRMMKYFLLEKNGKTVEGMGEDNNFYNELNIVFKGGEI
DGKVYEGVKTYLYYNEFRNYLTKKPFNEEKTKLNFDCGQILSGWDKNKESEKLGVILRKDNK
YYLAIINKKHNKIFDVKKNSYAYIVGDNFYEKMEYKLFPDAKRMIPKIAFAKNNKEKFGWTDE
IQKIKNEYAEFQEGKKNDKNLWKDKFNKNKMEKLITYYQNCLEKGGYKDIYNFRWKSPDKY
SGIGEFNDEIDRQSYCLKFVKVDFNYVFEKVKSGELYLFQIYNKDFSDKADRAQKENIHTEYFK
LLFDQRNLDNVVLKLSGGAEIFYRPKTEGLPKKKDNKGNEVVRHRRYADDKYFLHLPIQLNF
GRGNLSGGEFNSKINQYLSEQREIKIIGIDRGEKHLAYYSVINQDGKIEEIESLNTVNGIDYRKKL
DELEKKREQERKSWQSISKIKDLKKGYISHVIKKICDLAIEHNAIIVFEDLSGGFKNSRKKIEKQI
YQNLELALATKLNYLTFKDKNFGESGHYLNAYQLAPKIDNYQDIKMQTGIVFYTPAGYTSSTC
PQCGFRKTLKFDYTATISKAEDLIRGSKLNIVFEKEKNRFKINYLFNPIEKKKKKIKENELFADA
GAKNEFTIYSDVKRIKWHNTGTKRLEEAEGERLLENKNSRGRDKEYDINKCLTRLFRENKIDV
NGDIIGQITKIKSLKLYQDLFYYLFLATLIRNNVSGSDIDYIQCPSCHFHSDGGFQKQKENGDAN
GAYNIARKGILILKKIKQFAAQDKDMKNFGWKHLTVDINEWDKFTQK (SEQ ID NO: 245)
CasM.1895866
MNKNFSNFTELYTLSKTLRFELKPVAQTKENIKKGKFLESDKKKADDYKDVKKIIDNYHKFFI
DDVLKNASFDWTVLEKEMSDFNKSKADDSKVEAEQKKLRDQIAKKLTSDKRFKALTASTPSD
LFNKDKDFIDWFTQNSTKDINKEALETFKRFSSYFKGFQENRKNVYSAEPIPTAVPYRLVNDNF
PKFLQNIALFKIIQEKCPQVISDVEKELASYLGKEKLADIFTVQAFNKYLCQGGKENQRGIDFYN
NILGGIAEKEGGINLRGINQFLNLYWQQHQDFAKQNKRIKMIPLFKQILSDRSSLSFKIESINTDQ
ELLTSITEYADKLETKSNDEKKSVLDICSDLFASIKAQNLQEIYVNRKDINSISRILTGDWSWLQ
SRMNVYADEVFTTKAEKTRWQKSIDGDEGENKSKGVFSLAELNSVLEYSSENVSETDVRITDF
FDHRNRFYYEKESGLFKQGDELVALSIRESCEDILAKRKAMDEAFANVSENNSLRDNSEDVAK
IKIYLDCVQELLHRIKPLKVNGLGDPAFYAVFDTVYNSLSEVISLYNKTRNYITKKAANPEKYK
LNFDNPTLADGWDLNKEQANTSVLMRKDGMYYLGIMNPKDKPKFAEKYECGNEACYEKMI
YKQFDATKQIPKCSTQVKEVKKHFQSGATDSIILNDKSKFKLDLVITKEIWFLNNHVWNGEKF
VPKRESNETRPKKFQIGYYKQTGDLGGYKEALNIWISFCKTFLQSYISSSIYDYDFKESSNYDSL
DEFYNYLNATCYKLSFINIPEATISQMVSEGKLYLFQIYNKDFAPGASGMPNMHTLYWKNLFS
EENLKNVVLKLNGEAELFYRPAGIKEPVIHAKGSYLVNRITKDGEPIPEKIHDEIYRNANGKLES
LSKEATEYKASHKVVIKEAKHDIIKDRHYTEPKFLFHVPLTINFKASGNSYINENVRRFLKNNP
DVNVIGLDRGERHLIYLSLINQKGEIIKQFTFNEVERNKNGQVIKVNYHEKLDQREKVRGAAR
KSWQAIGKIAELKEGYLSAVIHQLTKLMVEYNAIVVMEDLNFGFKRGRFHVEKQVYQKFEHM
LIDKLNYLVFKDRGLTEAGGVLNGYQLAGQFESFQKLGKQSGMLFYVPAGYTSKIDPKTGFAS
MSNFKDLTNVHKKRAFFSKFDDIHFDDATGSFVFTFDYKNFDGKAKEEMKRTKWSVYSKDK
RIVYLSKTKSYEDVQPTEKIKASLESVGIEYMSGNNLIDSIMVIGAELKDGAKPSKEIADFWDRL
LYNFKLILQMRNSNAKTGEDYIISPVMADDGTFFDSREEFKKGENAKMPVDADANGAYHIAL
KGLSLLKRFDAASENELKKFDMKISNVDWFKFAQEKSYAE (SEQ ID NO: 246)
CasM.19498
LHETEKSLKFAEKYIAMPTITRKIELTLCTEGLSDEQRKEQWGLLYHINDNLYKAANNISSKLY
LDEHVSSMVRMKHAEYLSLQKELARAEKQKVDDAIIVELTRKLAVAEKEMTDQELAICKYAT
EMSTNTLAYNFAKEIETKIFGQILACLENNAHALFVDDSPNVRRGERAIRNYKKGMPIPFPWNR
SIKIEADGGEFYLRWYNGLRFLLTFGKDRSNNRLIVKRCMKMDEVFEGEYKLCNSSIQLAKRD
GKPKLFLLLVVNIPQEHVELNKNIVVGVDLGVNVPAYVATNITEERKAIGDREHFLNTRMTFQ
KRYKSLQRLKGTAGGKGRTKKLEPLERLRDAERNWVHTQNHLFSREVVNFAVQARAATINIE
DLSGFGKDNDGNADEKKEFVLRNWSYYELQNMITYKASKYGIKVEKIRPDYTSKTCSWCGQQ
GFREGVTFICENPECKQHGEKIHADYNAARNIANSKDIIKKNE (SEQ ID NO: 405)
CasM.19548
MAETKRLQKVAKFQIVKPVNMSWDELGRMLRDVRYRLSRLANMAVSETYQNLHQRYRLKN
QDAPKSLKIGQLSRNLRKILREEGVEEENLSKYSKTCVLPDTITGAFSRYKLSSIDWRKVLTGKI
SVPNYKTNLSIPIRCDKPHQRRLELTETGEIEADLMICNKPYPRVLLSTRTISDGQRTVLERLVSN
KTNFLPGYRHRFFEVKEKKGKWELSVTYDFPKAEATRLHPDIIVGVDLGWSVPLYAAINNGYA
RIGYRKFEPLAKRIKHLQKQIKGRRFSTQKGGVKDLAQPTARAGHGRKRILKPIEKLEYKIDNA
YTTLNHQLSHCVVEFAKNNGAGLIQIENLEGLKDDLSGTFIGQNWRYNQLQNFIKYKADEAGI
KVHPVNPCYTSRRCSHCGFIHISFDREYRDKNRKNGKATMFECPKGCKPLNADYNAAKNLAT
FDIEEKIRLQCKQQSIEYKELPKD (SEQ ID NO: 406)
CasM.19910
MPGTEKRLQKVATFEIVKPVNMSWPEFGKMLRDVRYRYWRLANMAVCENYMRFYQWRTQ
QTDANDRYKVKTLNRILRKMLIEEKNADEKELSRYSRDGAVSGYICGAFEKTKLSAVKSSSKW
KKVIAGKESLPLFKKDLAIPINCSDHQPRLIERTQSGEYEVDLRICQQPYPRVLLSTAKISDGQK
AILERLVSNETNSLPGYRHRFFEIKEKRNKWYLSVSYDFPKIDATRLHPNIIVGVDLGWSVPLY
AAISNGYARIGYRKLKALGDRIKALQRQTIARRRSIQRTGEQDLSAPTARSGHGRKRILHPIEKL
EGKIDNAYKTLNHQLSHCVIEFAKNHGAGLIQVENLKGLAEELSGTFIGQNWRYNQLQEFIKY
KAKEAGIEVKEVNPCYTSRRCSECGFIHKEFTFEYRQANKKTDKATMFECPKCGYKAIADYNA
ARNLANPDIAEKIRLQCKEQGIEYKELPKD (SEQ ID NO: 407)
CasM.19948
MPTITRKIELHFCTEGLSDEKQKEQRQLLYHINDNLYKAANNISSKLYLDEHVSSMVRLKHAD
YLSLQRELARAEKQKTPDDELITELSRKLSAAEKEMTDQELAICKYATEMATSTLAYNFAKEM
ETEIFGQILACLENNAHAVFVDDSLSVKRGERAIRNYKKGMPIPFPWNKNIKIETKDCEFYLRW
YNGIRFRLHFGKDRSNNRLIVQRCLKLDDNFESEYKLCNSSIQLDKRDGKTKLFLLLVVNIPQE
HVELNKNIVVGVDLGLNYPAYVATNSTEERKYIGDRDHFLKIRMQFQSRYKSLQRLKGTAGG
KGRAKKLEPLERLRKAERNWVHTQNHLFSRDVVNFAVQTHAATIHMEDLSGFGKDNDGNAD
EKKEFVLRNWSYYELQSMIEYKAAKYGIKVEKIRPAYTSKTCSWCGQQGDRKSTTFICENPEC
KHYGESIHADYNAARNIANSKDIVKKNE (SEQ ID NO: 408)
CasM.265291
MSKITRKIEIIPDIDGITHEESNKKCYNTFYKFDRKLYKVANLLVSQLYGLDNLLSLMRLQNDE
YVKCQSKLSFKSITDATKEEIKKRMQEIDAELVSMKNDIAPKHPQTYSYRAVTSSEYAKDIPSDI
LNNLKQDVYQHFNENKKEQIRGERSLATYKKGMPIPFSFEKRHVIICDGDNYYLPWFEDTRFR
LNFGRDRSNNRAIIDNCIKTKKYKLCAAAKIQLKERKLFLLITVDIPKAESVPVKGKVMGVDLG
VINPAYVAVNDGPERSRIGNGEAFQKQRDVFRRRFRELQRSQLTQGGHGRKHKTKATEILRGK
ERNWVQTENHRISREIVNLASRWKVETIQMESLKGFGKNQEGEVEYNHKRLLGRWSYFELQK
DIEYKAAMAGIAVQYVNPAYTSQTCHVCGQRGNRIERDTFICTNPECTCYNQAQDADMNAAI
NIAKSKDVIK (SEQ ID NO: 409)
CasM.270012
MPTITRKIELTLCTDGLSDEERKAQWGLLYHINDNLYKAANNISSKLYLDEHVSSMVRLKHAE
YLSLQKELAKAERQKMPDVDVIEELRERLSAAEQEMSDQELAICKYATEMSTNTLAYRFATEI
ETNIFGQILARLENNAQAVFLTDAPDVKRGERAIRNYKKGMPIPFPWNNSIKIECEGGEFYLRW
YSGLRFHFNFGKDRSGNRLIVQRCLKLDKEYDGEYKLCNSSIQMVKRDGSTKFFLLMVVNIPQ
EYVELNKHIVVGVDLGINVPAYVATNITPERKAIGDREHFLNTRMAFQRRYKSLQRLKTTAGG
KGRTKKLEPLERLRQAEHNWVHTQNHLFSREVVNFALQTHAATIHLEDLSGFGKDSDGNADE
RKEFVLRNWSYYELQNMITYKAAKYGIRVEKIRPAFTSRTCSCCGHEGFREGVTFICENPECQQ
FGEKVHADYNAARNIANSKDIIKKNE (SEQ ID NO: 410)
CasM.272451
MPTITRKIELTLCTEGLSEEQRKEQWGLLYHINDNLYKAANNISSKLYLDDHVSSMVRMKHAE
YLSLLKELARAEKQKTPDADAIAELRKKVAAAEKEMTDQEHAICKYATEMSTETLAYKFATEI
ETNVFGQILACLKQAAQSNFKNDAKDVKRGERAIRNYKKGMPIPFPWNDSLRIEKDNKDFYLR
WYNGLRFLFNFGKDRSNNRLIVERCLKMDADYDGEYKLCNSSIQIAKREGKVKLFLLLVVSIP
QEHVELNKKVVVGVDLGINVPAYVATNITEERKAIGDREHFLNTRMAFQRRYKSLQRLKGTA
GGKGRTKKLEPLERLRKAEHNWVHTQNHLFSREVVDFAVKTHAATIHMEDLSGFGKDNDGN
ADERKEFVLRNWSFYELQNMITYKAAKYGIKVEKIRPAYTSKTCSCCGRQGFRSGVTFICENPE
CKQYGEKVHADYNAARNIANSKEIIKKNE (SEQ ID NO: 411)
CasM.274429
MKNNVEEKRPDKEKRLTKVATFQIVKPVNMSWSEFGKMLRDVRYRLSRLANMAVSEAYQNL
HQRYRLKNQNAPKSVKIGQISRDLRKILLEEGLEEENLSKYSKMCVLPDTITGAFSRYKLSTID
WRKVLTGKISIPNYKANLSIPIRCDKPQQRRLERTETGEIEVDLMICNKPYPRVLLSTRTISDGQR
SVLERLVLNNANSLPGYRHRIFEIKEKRNEWYLSVTYDFPKAETTKLHSDIIVGVDLGWSVPLY
AAINNGYARIGYKQLKPLGDSIKALQRQTIARRRSIQRGGTQDLAAPTARSGHGIKRILQPIEKL
EGKIDNAYKTLNHQLSHCVIEFAKNHGAGVIQIENLKGLAEELSGTFIGQNWRYYQLQEFIKYK
AKEAGIIVKEVNPFYTSRRCSECGYIHKDFTFEYRQANRKNGKSTMFECPKKEEKGCKPLNAD
YNAARNLATSDIEDKIRLQCKEQGIEYKEIKEK (SEQ ID NO: 412)
CasM.277378
VTLLVKVVKIHLISEQFDKAGNRIDYKEVNKILWELQKQTREAKNKTVQLLWEWNNFSSDYV
KASGIYPKAKDIFGYSSVHGQANKELRTKLILNSSNLSTTTMDVCKIFNTYKKEVWEGKRSVPS
YKSDQPLDLHNDSIKLIYENKEFYVRLGLLNRAGFAKYGFKDGFRFKMQVKDNSTKTILERCF
DGIYTIVASKLLYDQKKNRWKLNLSYSFDNKNISELDKEKILGVDVGVSCPLVASVFGDRDRFI
IKGGEIEKFRKSVEARRRSMLEQTKYCGDGRIGHGRKKRTEPALNIGDKIARFRDTTNHKYSRA
LIEYAVKKGCGTIQMEKLTGITSKADRFLKDWTYYDLQTKIENKAKEVGINVVYIAPKYTSQR
CSKCGYIHKDNRPNQAKFRCLKCDFESNADYNASQNIGIKNIDKTIKKERKKQKSEAQVNEK
(SEQ ID NO: 413)
CasM.281050
MPDTDKGKRLTKVATFQIVKPVNMSWNEFGKMLHDVRYRYWRLANMAVCENYMRFYRWR
TQQTDTNDHYKVKIINGILRKMLIEEKNADEKELSRYSRDGAVSGYVYGAFTQTKLSAITSKSK
WGEVIKGKSALPLFKRDTSIPIMCTDKKPSMIEKTASGEYEVDLRICLKDKQLRPNGYPSVLLST
TKISDGQKAVLERLVSNKTNSLPGYRHRFFEVKEKRGDWYLSVSYDFPQAEATRLHPDIIVGV
DLGWSVPLYAAINNGYARIGWRKLEPLAKSIKHLQKQTIVRRRSFQKGGKKDLAASTARTGH
GIKRILQPIEKLEGKIDNAYKTLNHQLSHCIIEFAKNHGAGVIQIENLKGLAEELSGTFIGQNWR
YHQLQEFIKYKAEEAGIAVKEVNPRYTSRRCSKCGYIHIGFDREYRDKNRKNGKSTMFECPEC
SKRIKDYKPLNADYNAAKNLATADIEEKIRLQCKEQGIEYKELPKD (SEQ ID NO: 415)
CasM.285333
MPTITRKIKLELCTKGLSEEERKAQWNLLYHINDNLYRSANNISSKLYLDEHVSSLVWLKHKE
HQTLKADLAKAKKQKIQDEKTIAELESRLKSCESEMSDQELAICKYTDEMSSKTLSYKFATELE
LNIYAQILTQVQSKVYADFQNDQKDVRDGKRAIRTYKKGMPIPFPWRNNIRMEPVKKGREYE
FYIKWYNDIRFQLIFGKDRSNNRLILQRCFKLDENCVEDYQMRTSSIKMVKGANGTELFLYLV
VDIPQEKHILNNKIVVGVDLGINVPAYVATNVTDDRKAIGDREHFLNTRMAISKRFHSFQRLKG
TTGGRGKTKKLEPLERLKEKERNWVHTQNHLFSRDVITFALHVKAATIQMEDLSGYGKDDEG
NVVEEKKFLLGKWSYYELQEMIKYKAKKVGMRVNFIKPAYSSQTCSWCGERGERNSTSFVCT
NSECSHYGEDLHADYNAARNIARSKNIIRYE (SEQ ID NO: 416)
CasM.286285
MIITRKIQILFAAQGEEFKKDKDTLYKWSNIVHHASNIVASNKYVCDHLQGMVYLTEEGKEAV
SELSQKVDDIFNTSRMNTTYRMISSLYKGEIPTDILSCVNMQVSKLYNKERKKMADGDRSLRS
YRSNIPIPFSANSLMRKWKYADKEYSFDLFGIPFKVVLGKDKSNNRSILERLMDGTYKAATSSI
KIQNCEDETGKKTRKFFLLLCVEIPDKSYAGREDNILFAELSIDHPLLVSFPIKKEESKPIPIGNKQ
SYLYKRLQIQKGLDSCKASCKWNKGGRGRKRKMKSTERFKAKEHNFVDAYMHQISAALIKF
AIKHDIGKLCLVDVDKKIKEAKESPFVLRNWSYYSLLTKIQYKAKMNGITVVMVDKNVL
(SEQ ID NO: 417)
CasM.286678
MPTITRKIRLHLCTDGLSEEERKAQWKMLYRINDNLYRAANNISSKLYLDEHISSMVRLKHAE
YTSLKTELLKAKKADDEETVAELEARINVLNAELSAQEEAICSYATEMATRTLAGKFASELDL
NIYGQILAEVKSVVFKNFNSDSKDVREGKRSIRTYKKGMPIPFPWNKTIRLEAVKKESSSKHDE
DEYEVYLNWYKSSRTEKKAIRFRLDFGKDKSNNQQIVKRCLNLDNTSSESYQLQTSSIQMKKG
SEGAELYLLLVVNIPQDQHVLNKKIVVGVDLGINVPAYVATNCTEERKSIGDREHFLNARIAFH
RRFHSFQKLKGTTGGRGRKKKLEPLERLREKERNWVHTQNHLISRDVINFALQTKAATIQMED
LSGYGKDEEGNVKPENKFLQSRWSYFELQSMIKYKAAKCGIKVNLINPSYTSQTCSWCGQMG
VRESTSFVCQNPECKKYGKDIHADYNAARNIARSNKTVKNE (SEQ ID NO: 418)
CasM.287128
MPTITRKIELRLCTEGLSDEERKAQWMLLYHINDNLYRSANNISSKLYLDEHVSSMVRLKHAE
YQSTAAELLKAKKNNADEGTISTLEDKVETLKTEMSAQGIAICNYATEMATRTLAGKFASELE
LNIYGQILAEVKNVVHTNFTNDAKEVREGKRSIRTYKKGMPIPFPWNKSIKIEPVKASSQNEGQ
DDYEFYLKWYNGLKFILHFGKDRSNNRQILKRCFGLDNLCNERYQMRTSSIQMKKGSNGMEL
YLLLVLSIPKEQHSLNKKVVVGVDLGINVPAYVATNCTEERRAIGDREQFLNTRMAIIRRKHSF
QRLKGTAGGRGRKKKLDPLERLRETERNWVHTQNHLYSRDIIKFALETKAATIQMEKLKGFG
RDDNGNVIEEKKFLLGKWSYYELQNMIKYKAGKVGIKVNFIAPAYTSQTCSCCGVRDDRNRK
STSFICHNPDCQMYGKEIHADYNAARNIARSKNVIKDE (SEQ ID NO: 419)
CasM.287826
MPAITRKIELTLCTEGLSEEQRKEQWGLLYHINDNLYKAANNISSKLYLDEHVSSMVRMKHAD
YLSLLKELARAEKQKTPDDELIAELREKLSLAEQEMTDQELAICNYATEMATSTLAYNFAKEIE
TEIFGQILACLENNAHAVFVDDSPTVRRGERAIRNYKKGMPIPFPWNKSIRIVEKDGEFYLRWY
NGMRFLLTFGKDRSNNRIIMKRCLKMDQDFEGEYKLCNSSIQMVKREGKTKLFLLIVVNIPQE
HVELNKNIVVGVDLGVNVPAYVATNITEERKAIGDREHFLNTRMQFQRRYKSLQRLKGTAGG
KGRTKKLEPLERLRKAEHNWVHTQNHLFSREVVNFAVQTRAATIHMEDLSGFGKDNDGNAD
EQKEFVLRNWSFYELQNMIAYKAAKYGIKVEKVKPAYTSKTCSWCGQLGFRQGVTFICENPA
CKQCGEKVHADYNAARNIANSKDIIKKNE (SEQ ID NO: 420)
CasM.287896
MAGQRHTKVAKFQILKPAADMRWSELGRLLRDAQYRVYRLANLALSEKYLRFHLFRTGQTE
SLPECRIGRLNRQLRQMLKDEGGADDSVLDRFSRTGALPDTVVGALWQYRLHALTKGEKWN
KVTRGETALPTFRRSMALPIRCDKRIHHRLERAALDSVELDLMICTRPYPRVILKTAKLDDGAA
AILERLLDNEGQLLEGYRQRCFEVRYAEDEKAWWLHVTYDSPATPAPHLSKDIIVGVDLGFSC
PMYVALSNGDARLGRRQFAALAARIRSLQTQVMARRRQMLSGGKASLSGDTARSGHGRKRK
LLPIESLEGRINRAYTTLNHQLSISVVHFAVHHGAGVIQIENLEGLQNELTGTFLGQRWRYHQL
QEFLNYKANEAGIEVRRVNPRYTSRRCSKCGYIHVDFNRAFRDAARQEGKVARFCCPKCEYE
AHPDYNAARNLATVDIEGIIKVQCERQGIDRPSVENQDEVAK (SEQ ID NO: 421)
CasM.287936
MPTITRKIELTLCTEGLSDQERKDQWNLLYHINDNLYRAANNISSKLYLDDHVGSMVRLKHAE
YLSLLRALEKAKKQKAPDEEVIAELSQQVATAEQEMDEQAKAICQYATEMSTQTLSYRFATEL
ETNIFGQILTCLRQGVFSTFNSDARDVKRGERSIRTYKKGMPIPFPWNDSLRIGFEDGEFYLRW
YNGLRFRFDFGKDRSNNRLIVQRCMKMDKDYEGDYKLCNSSIQMVKREGKPKFFLLLVVNIP
QERVELNKNIVVGVDLGINAPAYVATNTTPERKQIGDREHFLNERMAFQRRFKSLQRLKSTTG
GRGRAKKLEPLERLRKAEQNWVHTQNHLFSREVIDFAVKARAATIHMEDLSGFGKDRDGNA
DERKEFVLRNWSYYELQNMITYKAAKYGIKVEKIRPAYTSKTCSWCGHQGFREGITFICENPE
CKKFGEKEHADYNAARNIANSKEIIKNNEE (SEQ ID NO: 422)
CasM.288450
MPTITRKIELSLCTDGLSDEQLKEQRQLLYHINDNLYRAANNVSSKLYLDEHVSSMVRLKHAD
YLSLLRDLARAEKQKSPDEALISELRSKLAAAQREMTEQELAICRYATEMSTQSLSYRFVTEME
THIFAKILDCLKQGVYATFNSDARDVKRGERSIRNYKKGMPIPFAWSDSVRIEQEADEFYLRW
YNGIRFRLVFGKDRSNNRLIVKRCLKLDKDYEGDYKLCNSSVQMVKREGKPKTFLLLVVKIPQ
EQVELNKKIVLGVDLGINYPVYAATNCTEERIYFGEREHFLNTRMQFQRRYKSLQRLKGTTGG
KGRKKKLEPLERLRKAERNWVHTQNHLFSQKTVDFALQTHAATIHLEDLSGFGRDSDGSAEE
KKEFVLRNWSYYELQQMITYKAAKYGIKVEKIRPAYTSQTCSWCGQRGFRQGVTFICENPECK
KCGEKEQADYNAARNIAKSKDVIKDDDE (SEQ ID NO: 423)
CasM.288712
MSIVTRKIELIPDIENLTHEESNQRCYKLLYNIDKKLYKLANLLVCQLFGLDNLLSLMRLQNDE
YVKFQSKLASKSISKETQKNIKEHMKEIDKELLARKAEIAPKSPLTFAYRAIKGSLYAKDLPSDI
FNTLKQDVFKHFNETKKEQLRGERSLATYKRGIPIPFSLMKKNVIVSEGDNYYLTWFEETRFKL
NFGKDRSNNRAIIDNCLKTNKYKLCTAAKIQLKNKKLFFLVTVDIPETKNTIIKGKVMGVDLG
VVHPAYVAVNDGPERSLIGDGDAFQKQRDVFRRRFKELQRCQLTQGGHGRKHKTKATEILRG
KERNWVQTENHRISRKIVNLAIRWKVESIQMENLKGFGKDSEGEVETKHKRLLGRWSYFELQ
KDIEYKAQKAGIKVVYINPAYTSQTCHVCGKKGDRTERDTFICLNTECSCYGKPQDADMNAAI
NIARSKNIVK (SEQ ID NO: 424)
CasM.289248
MPTITRKIELMLCSEGLSDEQRKEQWGLLYHINDNLYKAANNISSKLYLDEHVSSMVRMKHA
EYLSLLKELARAEKQQTPDEGLIAELSRKLSAAEKEMADQELAICKYATEMSTQTLSYNFAKEI
ETNIFGQILTCLRQGVYATFNSDAKDVKRGERAIRNYKKGMPIPFPWNKSLKIEAEGGDFYLR
WYNGLRFLLTFGKDRSNNRMIVKRCMKMDEDFEGEYKLCNSSIQLAKRDGKPKLFLLLVVNI
PQEHVELNKKIVVGVDLGVNVPAYVATNITEERKAIGDREHFLNTRMAFQRRYKSLQRLKGT
AGGKGRTKKLEPLERLRDAERNWVHTQNHLFSREVVNFAVQARAATIHMEDMSGFGKDKDG
NADEKKEFVLRNWSFYELQNMIAYKSAKYGIKVVKIRPAYTSKTCSWCGQQGERKSTTFICEN
PECKHYGESIHADYNAARNIANSNDIVKENE (SEQ ID NO: 425)
CasM.289726
MVITRKIEVFVCESDNDLRRSYYEKLYDIRNIAQEAANRATSMLYAIDNLIPCLDEDSRKLIQYI
GAKGTPASRQNAAYTIMSHLYKDRMPGIMDMLSNLAQYVTKNYSEDRKRGMYKNALRSYK
CSLPVPYQKKSFKGLRFNWYEDSDGDAHEGCFFSLAGVPLQMRFGRDRSNNRLIVERVISGEY
KMCTSSLKFDGKKLFLLLCVDIPKQEANVDPKKTLYAYLGVMNPIICTCDVRAKQEYDSGYK
CFEIGTKEEFNYRRRQIQEAVRRCQINNRYSSGGKGRKKKCQAIERWHEKEKNYVDTKLHTYS
RMLVDLAVAHKCGTIVLLNQKKREDKAKDDNQNGEPFVLRNWSYYNLKDKIGYKCKLAGIK
LVQDKEETEEE (SEQ ID NO: 426)
CasM.289802
MVITRKIEVFVCEDSKDLRKEYYDKIYKCRDIAVKTANLGVSHLFMLDNTTPYLSDDDREKLT
FLGCSGKKATKQNAPYVAASEKFKGQADMSMLSSVLQNVGKMYQDDKKKGMWSKSLRSY
KANMPIPFKASCYRNLRFADYNDKEDKPHNGCFFTLMGIPFQCKFGKDRSGNRIIMQAVVDG
KYKMCTSSLQIDGKKIFLLLCVDIPKKVVKLDESKTLYAFLGVMNPIVCTTDIKQKGDIDTDW
KLWEIGTEAEFNYRRRQIQEAVKRCQVNNRYSRGGHGRFAKTKAIERWRAVERNYVDTKLHT
YSKMLIDLAVKHKCGKIVLMNQLHREDAAKDDKFVLRNWSYHSLRTKIDYKAKMYGIKVEV
EK (SEQ ID NO: 427)
CasM.290380
MPVITRKIKLNLCTEGLSEDERKAQWKMLYRINDNLYRAANNISSKLYLDEHVSSMVRLKNA
EYTSLVSDLMKAKKAEDEAAITDLEAKIESLKSEMTAQEEAICCYATEMATRTLAGKFASELD
LDIYGQILAEVKSVVFKNFNSDSKEVREGNRSIRTYKKGMPIPFPWNKTIRLEAVKKELSGKHD
EDEYDFYLNWYKSSRTDKKAIRFRLYFGKDKSNNQQIVKRCLHLDSTSSENYQMQTSSIQMK
KGPEGAELYLLLVVNIPQEQHALNKKIVVGVDLGINVPAYVATNCTEERKAIGDRDHFLNTRM
AFSRRFHSFQRLKGTSGGKGRKKKLEPLERLREKERNWVHTQNHLISRDVINFALQVKAATIQ
MEDLSGYGKDEEGNVKPENKFLQSKWSYFELQSMIKYKAAKCGIKVNLIAPAYTSQTCSWCG
QMGIRESTSFVCQNPECKQYGKDIHADYNAARNIARSNKIVKNE (SEQ ID NO: 428)
CasM.293203
MEEKTKRLQKVAKFQIVKPVNMTWVELGKMLRDVRYRLWRLANMAVCENYMRFYQWRIG
KTDANENHKVKILNRRLREMIIEEKQADAKELMRYSRDGVVSGYICGAFEKIHLSAIKNKSKW
REVIRGKSNLPLFKRDLPIPINCSDHKPSLIAKTESDEYEVDLRICQKPYPRVLLSTAKISGGERAI
LERLVSNKTNSLPGYRHRFFEIKEKPKGRWNLHVTYDFARSEATMLHSDIIVGVDLGWSVPLY
AAVNKGHARIGWRKLEPLAKRIRHLQKQVKARRLSVQKGGQRDLAAPTARAGHGRKRILQPI
EKLEGKIDDAYKTLNHQLSHCVIEFAKNNGAGVIQVENLEGLKDTLTGTFIGQNWRYNQLQN
YIEYKAKEAGMELKKVNPCQTSQRCSNCGFIHRDFTFEYRQANKKNGKAAMFECPECSKKEN
YKPLNADYNAARNLATAGIEGKIRLQCEKQGIEYKGLPEE (SEQ ID NO: 430)
CasM.294190
MSKITRKIEIIPDIEGLTHDESNKKCYGAFYTFDKNLYKVANLLVSQLYGLDNLLSLMRLQNDE
YVKCQSKLSLKSTTDAEKENLKKRMKKIDAELVSIKNGMAPKHPQTFAYRAVTNCVYAKNIP
SDILNTLKQDVYKHFNDTKKEQFLGERSLTTYKRGMPVPFSIEKKHAIVCDGDNYYLPWFEDT
RFRLNFGRDKSNNRAIIDNCIKTKRYKLCAAAKIQLKDKKLFLLVTVDIPATETTSVKGKVMG
VDLGVVNPAYVAVNDGPERSRIGNGEAFQKQRDVFRRRFRELQRSQLTQGGHGRKHKTKATE
TLRGKERNWVQTENHRISREIVNLASRWKVECIQMESLKGYGKNQEGEVEDNHKRLLGRWS
YFELQKDIEYKAAMVGIQVKYINPAYTSQTCHVCGQRGNRIERDTFICTNPECTCYNQAQDAD
MNAAINIAKSKDVVK (SEQ ID NO: 431)
CasM.294406
MPTITRKIEMKLCTEGLSDQERKDQWNLLYHINDNLYRAANNISSKLYLDDHVLSMVRLKHA
EYLGLLRALEKAKKQKIPDEEVIAELSQKVAAAEQEMDDQAKAICQYATEMSTQSLSYRFATE
LETGIFTKILDCLKQGVFATFNSDTRDVKRGERSIRTYKKGMPIPFAWNDSLRIELEDGEFYLR
WYNGLRFRFDFGKDRSNNRLIVRRCLNMDEDYEGDYKLCNSSIQMVKREGLAKFFLLMVVNI
PQEQVELNKKIVVGVDLGINAPAYVATNITSERKQIGDREHFLNERMAFQRRFKSLQRLKGTT
GGRGRAKKLEPLERLRKAEQNWVHTQNHLFSREVIDFAVKSRAATIHMEDLSGFGKDRDGNA
DDKKEFVLRNWSYYELQSMITYKAAKYGIKVEKIRPAYTSKTCSWCGHQGFREGITFICENPE
CKKYGEKEHADYNAARNIANSIEIVKNNEE (SEQ ID NO: 432)
CasM.294655
MPFKVLKLKIIKPVNMDWNELGQSIRDTRYRVYRLANLAVSEAYLAFHLWRAGKTDAIPKAT
AGQLNRRLRDMLLEEARTKAVKDRKNTGEKGTEDDAKKAQKEMNKFSKTGALPDTVAGAL
FMYKVKGLISKGKWTQVIRGKSALPTFRNNMAIPIRCDKKTQRRLERTENGVELELMIRNKPY
PRVLLGTQGIGEGAEAIIERLLSNESQAEQGYKQRYFEVREDVNRTWWLYVCYALPASTPPRL
DPSKIVGVDLGFTCPMYAAISNGHARLGYRAFSSLAARVKALKLRTMRRRREIQRGGRTIVSG
EAARSGHGRKRKLLGIEKLQGRVNQAYTTLNHQMSAAVVKFAIENGAGTIQVENLEGLREEL
SGTFLGQMWRYFQLQEFLQYKAEENGIVIRKVNPRYTSRRCSQCGHINKEFTRKARDRNAEGG
YSAKFKCPDCEYEADADYNAAKNLAVDGIEGIIEKQCGSQGIVL (SEQ ID NO: 434)
CasM.295201
MFLYKELKTMAKTNAEEGKIENKEKRLTKVAKFQIVKPVNMTWPEFGKMLGDVRYRLSRVA
NMAVTEKYLESQQKRTGQKIQRENTLVTIANRKLREMLKKEKVKEEELDRYSRDGAVSGYVT
GPFEHNKLSAISKRFKEVLKGNMSLPNFKREMAIPINCSNAKLSTIEKTETGEYVVDLRISQKPW
PRVLLSTNRISNGQREILERLAANKTFSDDGYKHLFFEVKQQGKDWFLSVTYSFPKSEAPKLHK
DIIVGVDLGWSVPLYAAVNKGYARIGWQKFRPLAERIKHLQKQVKARRITIQKGGQQDLATPT
ARTGHGRKRILRPIEKLERKIENAYTTLNHQLSHCVIEFAKNNGAGVIQIENLSGLANELSGTYI
GQNWRYEQLQEYIRYKAEEAGIEVKHVNPCRTSQRCSECGFINDKFNFEYRQANRNNGMSAM
FECPECKKNKKDYKPINADYNAAKNLTTANIDEIIRLQCKKQGIEYKELPKD (SEQ ID NO:
435)
CasM.296640
MSKITRKIELIPDIENLTHEESNQRCYKVFYNIDNKLYKVANLLVCQLFGLDNLLSLMRLQNDE
YVKCQSKLASKSISEETKRDIKKRMEAIDKELLARKDEIAPKHPQTFAYRAIKDSDYAKDLPSDI
FNTLKQDVFKHFNETKKEQLRGERSLTTYKRGIPVPFNLMKKNVIVSDGDNYYLTWFEETRFK
LNFGKDRSNNRAIIDNCLKTNKYKLCTAAKIQLKNKKLFLLVTVDIPETKNKIIKGKVMGVDL
GVVHPAYVAVNDGPERSLIGDGDAFQKQRDVFRRRFRELQRCQLTQGGHGRKHKTKATENL
RGKERNWVQTENHRISREIVNLAIRWRVETIQMENLKGFGKDSDGDVETKHQRLLGRWSYFE
LQKDIEYKAAMAGIKVVYVNPAYTSQTCHVCGERGDRTERDTFICTNTECDCYGKPQDADMN
AAINIARSKNIVK (SEQ ID NO: 436)
CasM.296642
MTKVVKLPLICEQSDKDGNPIDYKKIYEILFELQRQTREIKNKSIQYCWEFSNFSSDYYKQNHE
YPKEKDILSYTLVGFVNDKFKTGNDLYSGNCSTTVRGACGEFKNSKTDFLKGTKSIINYKGNQ
PLDLHNKTIRFECIGKDYYAYLKLLNRPAFQRNNFSSSEIKFKVLVYDNSSKTIVERCIDNIYKIS
ASKLIYNEKKKCWVLNLSYSFTNNNVCELDENKILGVDLGIHYPICASVNGERKFFKIDGGEID
HTRRKIEVRKKSLLKQGSSCGEGRIGHGIKTRNKPVYNIEDKIACFRDTANHKYSRALINYAVN
NNCGIIQMEKLTGITADSDRFLKNWSYFDLQTKIEYKAKEAGITVVYIDPQYTSQRCSKCGYIS
KENRKVQAKFCCQKCGYEANADYNASQNIGIKDIDKIIKNTK (SEQ ID NO: 437)
CasM.298248
MNRIYQGRVSKVEIPDGKDEWKKLDDGESALWQHHQLFQDDVNYLLAAFAALVPTSCEDDI
WKDYQAAIERSWESYTGRQGIWDRPFENACVIVGCKKDASFKEFRRKLNSLTGSKASEKQKF
EALKQLFEPATEAAKKLKKHDEPVEESLKGKAKDLFGSTLVNLCAQKTKVTPRDVIAKQRNR
ASECTKKVNEGERLKWADVFYFKTDTSAAKWSREDAAKNIIQFLDKLLGEVEEKEKDAKTSD
QKKKMADLAERLEKQKKPLAAWCNNSKTDLPTTEPTRKGSGGYDLKAAVLFSLQPDLDGFR
DAFLLFNQARLKEEFATTEKGDAAYIARMAGGVARPVFPFFCDVWAGKVNDEKIGQGIWPDF
EKQAFSEVFTKIGQFIVRGRKFELRLAIADQIIAKIETQKKSDARLQAVERIAEDLADELPDTAV
DENGQKRPYGIRERTLKGWRKVRPAWREALKKTPNLTAEDLIKQKNRMQERQREKYGSASLF
DRLAKEPEIWNHDDKEDAVETWADYVENLEEKAHLETERLFAPAHATLSPRFFRWSETNNKE
HLEASSPDVPFELKADALDLSKKEKSQIKIHFWSPRLWRDGLRGKKENLDKDEPDQNWMPPV
LRAFVKARKWPCDKQSFAGASVRLAPRCKENIQLVFEPELHTEILSAKWKENFPFSPAKNKES
ESVGLFWPRTKEDKVLWFDKGETRCLGVDLGLTNSAAWQILQATNKDATAKAPRLRHRLNP
DSEKAAWFAHSITNGIVRVAGEDCWGWRKFAPDEKAKLRAELKKPAGKRNALCRKFLSLNR
EIEFETATHSFLPELSGSGGRNPTDDETKEAAEFFSTLKTKGFDITDRQPSWGKNLSFPKQNDEL
LWGLKRVRAQLFRLNRWSEQLGKERDSKPYQSAIEIIGNLRSDDPLIELATLKSEPKRLKSRIAE
LAGEYLDCFKTLLPRIADRILPWRRGHWSWKPCDNDWHRMELDASKPRPEALLAGQRGISLP
RLNQLKDLRQLAQSLNHLCRRKQIKRNETVPEPFEDCRQAMEDAREDRAKKIAHEVFAIALGV
ELAPPPPDKQERKQTESLHGVYRCLERGPVNFIALENLGGYNPSAKQGRRENRQLSSWLKGRI
HKILGELCEMVGMPIVLVNAEYTSRFSAKDHSPGFRAEEVQTDDSRRSFWQRKAKEEPSGWQ
NEFLCWLNKVPDGKSLLLPKKGGEFFVPLGEGTSLYHADLNAAYRIALRALAHRDRAELLGQ
TWIEKKPYLVDVAGVFPDSILRNGCAFKTISSSERLWEKVNGDLAMQRCREINLARFASWKIA
LPQQIISEALPPDEEDDIPM (SEQ ID NO: 439)
CasM.298264
MSEATKTLAYRYRLRLTPAQEDILDRSQEQLRLVWNHLVRSQHKVEHEWRHGRAASIKNELL
ELSLAKNATGQAIPSARKITEERGVSMEEALRLMRQKFVEKVSAIPLRKKDGSRCLRIARRKM
ATEYAVTVVNAKFKHYYGLGARMCKVLRDKFQKCSDMWIKGKFRRPRFKRKGESVALQRQ
VQSNSPFKLKRFSDLSALGGQALKKCEVIIHRPLPDSAEIKQIAVSGRRGQRHLIVMFKAASSD
VAKNFPATNRTAGVDPGIKVALTITPLDSPDFGTSDKIEKQPDLARDACFLKRLRRLQRKHDR
QRRQNNPECFDEKGRWIKGKRLHNESKNMQRTQSRITAMNTHLAESRRDFYHNAACEILRSF
DNVAVGKWRPAQTRQRKPTTPSPKGLGAARRATNRISYDHAISLFISYLKDKAERSVTTKHVQ
EVSEFGSTRSCPKCGKLTGPVGTEGLAVRDWTCVNCNTTFQRDAASAWQIAKRFKAEVASTS
QPAESQDSANSASVLTQV (SEQ ID NO: 440)
CasM.298446
MPTLTRKVELYVVGDKEEVSRVYDYIRLAMNATYKCFNECMTALYIAQVKEDTKEDRKELN
HLYSRQTYTKKETAFTNDIVFPEGLALAAYVNRMAQQKFVTSLKNGLMYGCVSLPTFKKDCA
VPLHVKFVSLAGEKGTNTGFYHEYADVNDLVNALEYDNSPKVFLRFPNNITFGVVFGNPYRG
REQRSVFSKIFLGEYKIQGSSIQINSRGKIILNLSMEVPKKKMEHIEGRVVGVDVGLAIPAMCAI
NDDDYTRSAIGNIDDFLKVRTQIQSQRRRLQKSLKNTSSGHGRTKKLKPLERIAEKERNFANTY
NHMVSKRVVDFAVKNGASQINIEDLSGFAKDKNGKSVEDDNMKRVLSNWSYFELQQQIRYK
AEQYDIKVRTVNPAYTSQTCSYCGQIGKRETQSKFVCTNPDCKCHKMYKKDWFNADFNAAR
NIALSTDYTDDEDGKKTKKKKSAKKKPEKKTEEA (SEQ ID NO: 441)
CasM.298612
MSGASGQITRDNKAQRSGPNKGEMSEDHSSTKRPKRVVKVAKYRIIKPVGEMTWPELGEILRT
VRYRVFRLGNLAVSEAYLNFHAFRTGKAEEFKSETIGKLSRRLRDMLISEGVKKEDIDRYSATG
AVPDTVAGALGQYKVRGITSPAKWRQVIRGTVSLPTFRNDMAIPVRCDKPAQRRLEKAKSEE
VEVDLMICRKPYPRVLIGTADLGGGQQAILERLLDNKDNSSDGYRQRLFEIKQDTQSKKWFLF
VTYDFPSSGALPLDPNVAVGVDLGVSVPLYAAINNGHARLGRRQFQALGSRIRSLQTQVDARR
RAIQRGGRSDVSQSTARSGHGVRRKLQPTEKLRKRIDRSYSTLNHQLSAAVVEFAKNQGAGT
VQMEDLGGLREELTGTFIGARWRYHQLQQFLEYKCDEAGITLNKVNPMYTSRRCSECGFIDK
DFDRAFRDRSRSDGRVARFICPECSYEADPDYNAARNIATLDIDKLIRVQCQKQGLKYDAL
(SEQ ID NO: 442)
CasM.299584
MIITRKIELWLSEDDNELRKAKWSYLKELNDEVYRAANFIVNNQYFNEILENRVIMQDTRLIDI
DSEIRKLYKSREKNKEKIDELKKIKKIRYQEAKNFYQTSKQNVTYQLTSREFPNIPANIVTSLNA
SIIKTLKTEWNEIKSGKRAVRNYRKGMPIPFNFSSSQKWFENKGEDIFLNWLGGLKFKLFFGRD
KSNNRAIVERAINKEYKYADSSIQLKDKKIFLLFVVDIPYEKANLNKNIAAGVDLGIAFPAFCAL
SEGYSRLSIGNKEDLLKVRLQMQSRRKRLQKALKITSGGKGRTKKLKALESLINKEKNYVTTY
NHKVSYQVIKFAKDNKAGIIKLEFLEGFGEDEKNKFILRNWSFYQLQKMIEYKAKREGIEVLYI
DPYHTSQTCAICGNYEEGQREKQEDFICKNPECKNFEKIVNADYNAALNIAKSNKIVSSSEQCE
YNKKHENNVL (SEQ ID NO: 443)
CasM.286251 (D267A)
MPTITRKIELTLCTEGLSDQERKDQWNLLYHINDNLYRAANNISSKLYLDDHVGSMVRLKHAE
YLSLLRALEKAKKQKAPDEEVIAELSQQVATAEQEMDEQAKAICQYATEMSTQTLSYRFATEL
ETNIFGQILTCLRQGVFSTFNSDARDVKRGERSIRTYKKGMPIPFPWNDSLRIGFEDGEFYLRW
YNGLRFRFDFGKDRSNNCLIVQRCMKMDKDYEGDYKLCNSSIQMVKREGKPKFFLLLVVNIP
QERVELNKNIVVGVALGINAPAYVATNTTPERKQIGDREHFLNERMAFQRRFKSLQRLKGTTG
GRGRAKKLEPLERLRKAEQNWVHTQNHLFSREVIDFAVKARAATIHMEDLSGFGKDNDGNA
DERKEFVLRNWSYYELQNMITYKAAKYGIKVEKIRPAYTSKTCSWCGHQGFREGITFICENPE
CKKFGEKEHADYNAARNIANSKEIIKNNEE (SEQ ID NO: 444)
CasM.19952 (D267A)
MPTITRKIELTLLTEGLSEEQRKEQWGLLYHINDNLYKAANNISSKLYLDDHVSSMVRMKHAE
YLSLLKELARAEKQKTPDADAIAELRKKVAAAEKEMTDQEHAICKYATEMSTQSLSYRFATEL
ETNIFAKILDCLKQGVFATFNSDARDVKRGERAIRNYKKGMPIPFAWDKSLRIEKDNKDFYLR
WYNGLRFLFNFGKDRSNNRLIVERCLKMDADYDGEYKLCNSSIQIAKREGKTKLFLLLVVKIP
QEHVELNKKVVVGVALGINVPAYVATNITEERKAIGDREHFLNSRMAFQRRYKSLQRLRGTA
GGKGRAKKLEPLERLRKAEHNWVHTQNHLFSREVVDFAVKSHAATIHMEDLSGFGKDNDGN
ADERKEFVLRNWSYYELQNMIAYKAAKYGIKVEKIHPAYTSKTCSWCGQLGFREGVTFICENP
ECKQCGEKVHADYNAARNIANSKDIIKKNE (SEQ ID NO: 445)
CasM.19952 (D267N)
MPTITRKIELTLLTEGLSEEQRKEQWGLLYHINDNLYKAANNISSKLYLDDHVSSMVRMKHAE
YLSLLKELARAEKQKTPDADAIAELRKKVAAAEKEMTDQEHAICKYATEMSTQSLSYRFATEL
ETNIFAKILDCLKQGVFATFNSDARDVKRGERAIRNYKKGMPIPFAWDKSLRIEKDNKDFYLR
WYNGLRFLFNFGKDRSNNRLIVERCLKMDADYDGEYKLCNSSIQIAKREGKTKLFLLLVVKIP
QEHVELNKKVVVGVNLGINVPAYVATNITEERKAIGDREHFLNSRMAFQRRYKSLQRLRGTA
GGKGRAKKLEPLERLRKAEHNWVHTQNHLFSREVVDFAVKSHAATIHMEDLSGFGKDNDGN
ADERKEFVLRNWSYYELQNMIAYKAAKYGIKVEKIHPAYTSKTCSWCGQLGFREGVTFICENP
ECKQCGEKVHADYNAARNIANSKDIIKKNE (SEQ ID NO: 446)
CasM.19952 (E363Q)
MPTITRKIELTLLTEGLSEEQRKEQWGLLYHINDNLYKAANNISSKLYLDDHVSSMVRMKHAE
YLSLLKELARAEKQKTPDADAIAELRKKVAAAEKEMTDQEHAICKYATEMSTQSLSYRFATEL
ETNIFAKILDCLKQGVFATFNSDARDVKRGERAIRNYKKGMPIPFAWDKSLRIEKDNKDFYLR
WYNGLRFLFNFGKDRSNNRLIVERCLKMDADYDGEYKLCNSSIQIAKREGKTKLFLLLVVKIP
QEHVELNKKVVVGVDLGINVPAYVATNITEERKAIGDREHFLNSRMAFQRRYKSLQRLRGTA
GGKGRAKKLEPLERLRKAEHNWVHTQNHLFSREVVDFAVKSHAATIHMQDLSGFGKDNDGN
ADERKEFVLRNWSYYELQNMIAYKAAKYGIKVEKIHPAYTSKTCSWCGQLGFREGVTFICENP
ECKQCGEKVHADYNAARNIANSKDIIKKNE (SEQ ID NO: 447)

In some embodiments, the effector protein comprises an amino acid sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 alterations relative to any one of sequences recited in TABLE 1.

In some embodiments, the effector protein comprises an amino acid sequence having at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 alterations relative to any one of sequences recited in TABLE 1.

The one or more alterations may be located at one or more positions located in a region of the polypeptide (e.g., effector protein) that comprises substrate binding activity, catalytic activity, and/or binding affinity for a substrate such as a target nucleic acid, an guide nucleic acid or a guide nucleic acid-target nucleic acid heteroduplex. The one or more alterations may be located at one or more positions corresponding to the one or more positions in any one of sequences recited in TABLE 1. As used herein, the phrase “a residue corresponding to position X in SEQ ID NO: Y” refers to a residue at a corresponding position following an alignment of two sequences.

In some embodiments, compositions, systems and methods described herein comprise a viral vector comprising a nucleic acid encoding an effector protein, wherein the amino acid sequence of the effector protein comprises an effector protein, wherein the amino acid sequence of the effector protein comprises at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500, at least about 520, at least about 540, at least about 560, at least about 580, at least about 600, at least about 620, at least about 640, at least about 660, at least about 680, at least about 700, at least about 720, at least about 740, at least about 760, at least about 780, at least about 800, at least about 850, at least about 900, at least about 950, at least about 1000, at least about 1050, at least about 1100, at least about 1150, at least about 1200, at least about 1250, at least about 1300, or at least about 1350 contiguous amino acids of any one of the sequences recited in TABLE 1.

As use herein, the terms “endonuclease” and “nuclease” are used interchangeably. In some embodiments, the endonuclease possesses nuclease activity (e.g., catalytic activity) for nucleic acid cleavage (e.g., ribonuclease activity (ribonucleic acid cleavage), or deoxyribonuclease activity (deoxyribonucleic acid cleavage), etc.). In some embodiments, the effector proteins function as an endonuclease that catalyzes cleavage within a target nucleic acid. In some embodiments, the effector proteins are capable of catalyzing non-sequence-specific cleavage of a single stranded nucleic acid. In some embodiments, the effector proteins (e.g., the effector proteins having the sequence of TABLE 1) are activated to perform trans cleavage activity after binding of a guide nucleic acid with a target nucleic acid. This trans cleavage activity may also be referred to as “collateral” or “transcollateral” cleavage. Trans cleavage activity may be non-specific cleavage of nearby single-stranded nucleic acid by the activated effector protein, such as trans cleavage of detector nucleic acids with a detection moiety.

Effector proteins disclosed herein may function as an endonuclease that catalyzes cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some instances, the target nucleic acid is single-stranded DNA. In some instances, the target nucleic acid is single-stranded RNA. The effector proteins may provide cis cleavage activity, trans cleavage activity, nickase activity, or a combination thereof. Cis cleavage activity is cleavage of a target nucleic acid that is hybridized to a guide nucleic acid (e.g., a dual gRNA or a sgRNA), wherein cleavage occurs within or directly adjacent to the region of the target nucleic acid that is hybridized to guide RNA. Trans cleavage activity (also referred to as transcollateral cleavage) is cleavage of ssDNA or ssRNA that is near, but not hybridized to the guide nucleic acid. Trans cleavage activity is triggered by the hybridization of the guide nucleic acid to the target nucleic acid. Nickase activity is a selective cleavage of one strand of a dsDNA. In some embodiments, trans cleavage is in reference to cleavage (hydrolysis of a phosphodiester bond) of one or more nucleic acids by an effector protein that is complexed with a guide nucleic acid and a target nucleic acid. The one or more nucleic acids may include the target nucleic acid as well as non-target nucleic acids. Trans cleavage may occur near, but not within or directly adjacent to, the region of the target nucleic acid that is hybridized to the guide nucleic acid. Trans cleavage activity may be triggered by the hybridization of the guide nucleic acid to the target nucleic acid.

Effector proteins of the present disclosure, dimers thereof, and multimeric complexes thereof may cleave or nick a target nucleic acid within or near a PAM sequence of the target nucleic acid. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer sequence. In some embodiments, a PAM is a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid. TABLE 1.1 provides exemplary PAM sequences for an effector protein.

TABLE 1.1
Exemplary PAM Sequences for
an Effector Proteins

| Effector | | Effector | | Effector | |
|---|---|---|---|---|---|
| Protein | | Protein | | Protein | |
| SEQ | PAM | SEQ | PAM | SEQ | PAM |
| ID NO: | Sequence | ID NO: | Sequence | ID NO: | Sequence |
| 1 | CTT | 43 | TTC | 243 | TTTT |
| 4 | TTC, | 44 | TTY | 246 | TTTN |
| | TTTC | | | | |
| 5 | TTY | 45 | TTY, | 405 | RTTN, |
| | | | TY, TTC | | TCG, |
| | | | | | RTTR, |
| | | | | | KRTTN |
| 8 | TTC, | 49 | TYN | 406 | CCN, |
| | YTN | | | | CCR |
| 9 | GNNN | 56 | TTN | 407 | TTYN, |
| | | | | | WTTYN |
| 12 | YTTR, | 57 | TYN, | 408 | RTTN |
| | TTYN | | TTN | | |
| 13 | CTT | 60 | TYN | 409 | TG, |
| | | | | | TNTG, |
| 14 | CC | 61 | TTN | 410 | GRTT, |
| | | | | | ANRTT |
| 15 | CC | 65 | TTN | 411 | RTTR, |
| | | | | | RTWG |
| 16 | CC | 66 | TTG | 412 | CCN |
| 18 | CC | 69 | TYN | 413 | TTYN, |
| | | | | | YN, |
| | | | | | YTTR |
| 19 | CC | 70 | TYN | 415 | TTTN |
| 20 | CC | 71 | TTN | 416 | GTYG, |
| | | | | | RGTYG |
| 21 | TC | 72 | RTTS | 418 | RTRG |
| 22 | TCG | 73 | TTN | 419 | RTNG |
| 23 | TCG, | 74 | TTN | 420 | RTTN |
| | KYG | | | | |
| 24 | TCG | 75 | VTTN | 422 | RTTR |
| 25 | RTTR | 76 | TTN | 423 | TCG, |
| | | | | | KCG |
| 26 | TCG | 77 | TYN, | 424 | TG, |
| | | | GTTN | | WNTG |
| 28 | RTTR | 78 | RTTN | 425 | RTTR |
| 29 | RTTG, | 79 | TTS | 428 | RTRG |
| | RTTR | | | | |
| 30 | TCG, | 80 | TTS | 430 | TYN |
| | RTTR | | | | |
| 31 | RTTR | 82 | TTN | 431 | TG, |
| | | | | | TNTG, |
| | | | | | WYTG, |
| | | | | | WNTG, |
| | | | | | TNTG |
| 32 | TCG, | 83 | TWN | 432 | TCG, |
| | KCG | | | | RTTR |
| 33 | KNTK, | 84 | RTTN | 434 | CCN, |
| | KNTT | | | | CCR |
| 34 | RTTR | 87 | TTN | 435 | TYN, |
| | | | | | WWTTN, |
| | | | | | TTTYN |
| 35 | TTC, | 88 | TTS | 436 | TG, |
| | YTC | | | | TNTG, |
| | | | | | WNTG |
| 36 | TTC, | 89 | TTN | 437 | TTC, |
| | TTCN | | | | TTNY |
| 37 | TTY, | 108 | TTTR | 439 | TCG, |
| | TY | | | | RTTR |
| 38 | TTC, | 110 | TTTR | 440 | RTTR |
| | TTCN | | | | |
| 39 | TYYT, | 112 | TTTR | 441 | TCG |
| | YN, | | | | |
| | CTTN, T | | | | |
| 40 | TTC | 240 | YYN | 442 | CC |
| 41 | YT, | 242 | TTTR | 443 | TTR, |
| | WNCT | | | | WTTR, |
| | | | | | RTRG |
| 42 | TTC, | | | | |
| | TTYN, | | | | |
| | TYYW | | | | |

In some embodiments, viral vectors described herein comprise a nucleotide sequence that encodes an effector protein, wherein the effector protein recognizes a corresponding PAM sequence as identified in TABLE 1.1, and wherein the amino acid sequence of the effector protein is at least 65%, at least 70%, at least 75%, at least 80%, at least 85%, at least 90%, at least 95%, at least 97%, or at least 98%, at least 99%, or 100% identical to any one of the amino acid sequences recited in TABLE 1.

In some embodiments, viral vectors described herein comprise a nucleic acid encoding an effector protein, wherein the effector protein comprises six amino acid sequences selected from the group comprising: (i) an amino acid sequence that is at least 60%, at least 70%, at least 80%, at least 90%, at least 95%, at least 98%, at least 99% or 100% identical to SEQ ID NO: 448 (shown in TABLE 11), (ii) an amino acid sequence that is at least 60%, at least 70%, at least 80%, at least 90%, at least 95%, at least 98%, at least 99% or 100% identical to SEQ ID NO: 449 (shown in TABLE 11), (iii) an amino acid sequence that is at least 60%, at least 70%, at least 80%, at least 90%, at least 95%, at least 98%, at least 99% or 100% identical to SEQ ID NO: 450 (shown in TABLE 11), (iv) an amino acid sequence that is at least 60%, at least 70%, at least 80%, at least 90%, at least 95%, at least 98%, at least 99% or 100% identical to SEQ ID NO: 451 (shown in TABLE 11), (v) an amino acid sequence that is at least 60%, at least 70%, at least 80%, at least 90%, at least 95%, at least 98%, at least 99% or 100% identical to SEQ ID NO: 452 (shown in TABLE 11), (vi) an amino acid sequence that is at least 60%, at least 70%, at least 80%, at least 90%, at least 95%, at least 98%, at least 99% or 100% identical to SEQ ID NO: 453 (shown in TABLE 11), and (vii) an amino acid sequence that is at least 60%, at least 70%, at least 80%, at least 90%, at least 95%, at least 98%, at least 99% or 100% identical to SEQ ID NO: 454 (shown in TABLE 11).

MEME_1 to MEME_7 are PROSITE motifs, a format which is routinely used in the art to describe a consensus sequence. For example, the PROSITE sequence [NH]AD corresponds to the sequences NAD and HAD. When an amino acid sequence is analyzed to calculate the degree of identity to the PROSITE sequence [NH]AD, both NAD and HAD are given equal weight. In other words, both NAD and HAD share 100% identity with the PROSITE motif [NH]AD.

In some embodiments, viral vectors described herein comprise a nucleic acid encoding an effector protein, wherein the effector protein comprises seven amino acid sequences selected from the group: (i) an amino acid sequence that is at least 40%, at least 50%, at least 60%, at least 70%, at least 80%, at least 90%, at least 95%, at least 98%, at least 99% or 100% identical to SEQ ID NO: 448, (ii) an amino acid sequence that is at least 40%, at least 50%, at least 60%, at least 70%, at least 80%, at least 90%, at least 95%, at least 98%, at least 99% or 100% identical to SEQ ID NO: 449, (iii) an amino acid sequence that is at least 40%, at least 50%, at least 60%, at least 70%, at least 80%, at least 90%, at least 95%, at least 98%, at least 99% or 100% identical to SEQ ID NO: 450, (iv) an amino acid sequence that is at least 40%, at least 50%, at least 60%, at least 70%, at least 80%, at least 90%, at least 95%, at least 98%, at least 99% or 100% identical to SEQ ID NO: 451, (v) an amino acid sequence that is at least 40%, at least 50%, at least 60%, at least 70%, at least 80%, at least 90%, at least 95%, at least 98%, at least 99% or 100% identical to SEQ ID NO: 452, (vi) an amino acid sequence that is at least 40%, at least 50%, at least 60%, at least 70%, at least 80%, at least 90%, at least 95%, at least 98%, at least 99% or 100% identical to SEQ ID NO: 453, and (vii) an amino acid sequence that is at least 40%, at least 50%, at least 60%, at least 70%, at least 80%, at least 90%, at least 95%, at least 98%, at least 99% or 100% identical to SEQ ID NO: 454.

In some embodiments, viral vectors described herein comprise a nucleic acid encoding an effector protein, wherein the effector protein comprises six amino acid sequences selected from the group: (i) an amino acid sequence that is at least 69.5% identical to SEQ ID NO: 448, (ii) an amino acid sequence that is at least 69.5% identical to SEQ ID NO: 449, (iii) an amino acid sequence that is at least 69.5% identical to SEQ ID NO: 450, (iv) an amino acid sequence that is at least 69.5% identical to SEQ ID NO: 451, (v) an amino acid sequence that is at least 69.5% identical to SEQ ID NO: 452, (vi) an amino acid sequence that is at least 69.5% identical to SEQ ID NO: 453, and (vii) an amino acid sequence that is at least 69.5% identical to SEQ ID NO: 454. In further preferred embodiments, the effector protein comprises six amino acid sequences selected from the group: (i) an amino acid sequence that is at least 80% identical to SEQ ID NO: 448, (ii) an amino acid sequence that is at least 80% identical to SEQ ID NO: 449, (iii) an amino acid sequence that is at least 80% identical to SEQ ID NO: 450, (iv) an amino acid sequence that is at least 80% identical to SEQ ID NO: 451, (v) an amino acid sequence that is at least 80% identical to SEQ ID NO: 452, (vi) an amino acid sequence that is at least 80% identical to SEQ ID NO: 453, and (vii) an amino acid sequence that is at least 80% identical to SEQ ID NO: 454.

In some embodiments, viral vectors described herein comprise a nucleic acid encoding an effector protein, wherein the amino acid sequence of the effector protein is (1) at least 50%, at least 60%, at least 70%, at least 75%, at least 80%, at least 85%, at least 90%, at least 95%, at least 98%, at least 99% or 100% identical to SEQ ID NO: 23, preferably at least 68% identical to SEQ ID NO: 23, and (2) includes six amino acid sequences selected from the group: (i) an amino acid sequence that is at 69.5% identical to SEQ ID NO: 448, (ii) an amino acid sequence that is at 69.5% identical to SEQ ID NO: 449, (iii) an amino acid sequence that is at 69.5% identical to SEQ ID NO: 450, (iv) an amino acid sequence that is at 69.5% identical to SEQ ID NO: 451, (v) an amino acid sequence that is at 69.5% identical to SEQ ID NO: 452, (vi) an amino acid sequence that is at 69.5% identical to SEQ ID NO: 453, and (vii) an amino acid sequence that is at 69.5% identical to SEQ ID NO: 454.

In some embodiments, viral vectors described herein comprise a nucleic acid encoding an effector protein, wherein the amino acid sequence of the effector protein is (1) at least 50%, at least 60%, at least 70%, at least 75%, at least 80%, at least 85%, at least 90%, at least 95%, at least 98%, at least 99% or 100% identical to SEQ ID NO: 23 and (2) includes six amino acid sequences selected from the group comprising: (i) an amino acid sequence that is at least 60%, at least 70%, at least 80%, at least 90%, at least 95%, at least 98%, at least 99% or 100% identical to SEQ ID NO: 448, (ii) an amino acid sequence that is at least 60%, at least 70%, at least 80%, at least 90%, at least 95%, at least 98%, at least 99% or 100% identical to SEQ ID NO: 449, (iii) an amino acid sequence that is at least 60%, at least 70%, at least 80%, at least 90%, at least 95%, at least 98%, at least 99% or 100% identical to SEQ ID NO: 450, (iv) an amino acid sequence that is at least 60%, at least 70%, at least 80%, at least 90%, at least 95%, at least 98%, at least 99% or 100% identical to SEQ ID NO: 451, (v) an amino acid sequence that is at least 60%, at least 70%, at least 80%, at least 90%, at least 95%, at least 98%, at least 99% or 100% identical to SEQ ID NO: 452, (vi) an amino acid sequence that is at least 60%, at least 70%, at least 80%, at least 90%, at least 95%, at least 98%, at least 99% or 100% identical to SEQ ID NO: 453, and (vii) an amino acid sequence that is at least 60%, at least 70%, at least 80%, at least 90%, at least 95%, at least 98%, at least 99% or 100% identical to SEQ ID NO: 454.

In some embodiments, viral vectors described herein comprise a nucleic acid encoding an effector protein, wherein the amino acid sequence of the effector protein is (1) at least 68% identical to SEQ ID NO: 23, and (2) includes six amino acid sequences selected from the group: (i) an amino acid sequence that is at least 69.5% identical to SEQ ID NO: 448, (ii) an amino acid sequence that is at least 69.5% identical to SEQ ID NO: 449, (iii) an amino acid sequence that is at least 69.5% identical to SEQ ID NO: 450, (iv) an amino acid sequence that is at least 69.5% identical to SEQ ID NO: 451, (v) an amino acid sequence that is at least 69.5% identical to SEQ ID NO: 452, (vi) an amino acid sequence that is at least 69.5% identical to SEQ ID NO: 453, and (vii) an amino acid sequence that is at least 69.5% identical to SEQ ID NO: 454.

In some embodiments, viral vectors described herein comprise a nucleic acid encoding an effector protein, wherein the amino acid sequence of the effector protein is at least 37%, at least 40%, at least 50%, at least 60%, at least 70%, at least 80%, at least 90%, at least 95%, at least 98%, at least 99% or 100% identical to SEQ ID NO: 451.

In some embodiments, viral vectors described herein comprise a nucleic acid encoding an effector protein, wherein the effector protein comprises (1) an amino acid sequence that is at least 37%, at least 40%, at least 50%, at least 60%, at least 70%, at least 80%, at least 90%, at least 95%, at least 98%, at least 99% or 100% identical to SEQ ID NO: 451, and (2) four amino acid sequences selected from the group: (i) an amino acid sequence that is at least 60%, at least 70%, at least 80%, at least 90%, at least 95%, at least 98%, at least 99% or 100% identical to SEQ ID NO: 448, (ii) an amino acid sequence that is at least 60%, at least 70%, at least 80%, at least 90%, at least 95%, at least 98%, at least 99% or 100% identical to SEQ ID NO: 449, (iii) an amino acid sequence that is at least 60%, at least 70%, at least 80%, at least 90%, at least 95%, at least 98%, at least 99% or 100% identical to SEQ ID NO: 450, (iv) an amino acid sequence that is at least 60%, at least 70%, at least 80%, at least 90%, at least 95%, at least 98%, at least 99% or 100% identical to SEQ ID NO: 452, (v) an amino acid sequence that is at least 60%, at least 70%, at least 80%, at least 90%, at least 95%, at least 98%, at least 99% or 100% identical to SEQ ID NO: 453, and (vi) an amino acid sequence that is at least 60%, at least 70%, at least 80%, at least 90%, at least 95%, at least 98%, at least 99% or 100% identical to SEQ ID NO: 454. In some further instances, the effector protein comprises an amino acid sequence that is at least 50%, at least 60%, at least 70%, at least 75%, at least 80%, at least 85%, at least 90%, at least 95%, at least 98%, at least 99% or 100% identical to SEQ ID NO: 23, preferably wherein the amino acid sequence is at least 68% identical to SEQ ID NO: 23.

In some embodiments, viral vectors described herein comprise a nucleic acid encoding an effector protein, wherein the effector protein comprises (1) an amino acid sequence that is at least 37%, at least 40%, at least 50%, at least 60%, at least 70%, at least 80%, at least 90%, at least 95%, at least 98%, at least 99% or 100% identical to SEQ ID NO: 451, and (2) four amino acid sequences selected from the group: (i) an amino acid sequence that is at least 69.5% identical to SEQ ID NO: 448, (ii) an amino acid sequence that is at least 69.5% identical to SEQ ID NO: 449, (iii) an amino acid sequence that is at least 69.5% identical to SEQ ID NO: 450, (iv) an amino acid sequence that is at least 69.5% identical to SEQ ID NO: 452, (v) an amino acid sequence that is at least 69.5% identical to SEQ ID NO: 453, and (vi) an amino acid sequence that is at least 69.5% identical to SEQ ID NO: 454. In some further instances, the effector protein comprises an amino acid sequence that is at least 50%, at least 60%, at least 70%, at least 75%, at least 80%, at least 85%, at least 90%, at least 95%, at least 98%, at least 99% or 100% identical to SEQ ID NO: 23, preferably wherein the amino acid sequence is at least 68% identical to SEQ ID NO: 23.

In some embodiments, viral vectors described herein comprise a nucleic acid encoding an effector protein, wherein the effector protein comprises one or more of: (i) an amino acid sequence that is at least 80%, preferably at least 90%, identical to SEQ ID NO: 448, (ii) an amino acid sequence that is at least 80%, preferably at least 90%, identical to SEQ ID NO: 449, (iii) an amino acid sequence that is at least 80%, preferably at least 90%, identical to SEQ ID NO: 450, (iv) an amino acid sequence that is at least 80%, preferably at least 90%, identical to SEQ ID NO: 451, (v) an amino acid sequence that is at least 80%, preferably at least 90%, identical to SEQ ID NO: 452, (vi) an amino acid sequence that is at least 80%, preferably at least 90%, identical to SEQ ID NO: 453, and (vii) an amino acid sequence that is at least 80%, preferably at least 90%, identical to SEQ ID NO: 454.

In some embodiments, viral vectors described herein comprise a nucleic acid encoding an effector protein, wherein the effector protein comprises amino acid sequences that have at least a threshold identity referred to herein to any one of SEQ ID NO: 448 to SEQ ID NO: 454 and the amino acid sequences are in the following order, starting from the N-terminus: (i) the sequence having at least the threshold identity with SEQ ID NO: 451, (ii) the sequence having at least the threshold identity with SEQ ID NO: 452, (iii) the sequence having at least the threshold identity with SEQ ID NO: 450, (iv) the sequence having at least the threshold identity with SEQ ID NO: 454, (v) the sequence having at least the threshold identity with SEQ ID NO: 449, (vi) the sequence having at least the threshold identity with SEQ ID NO: 448, and (vii) the sequence having at least the threshold identity with SEQ ID NO: 453. In some embodiments, viral vectors described herein comprise a nucleic acid encoding an effector protein, wherein the effector protein does not include an amino acid that meets a specified degree of identity (i.e., the threshold identity) with any one of SEQ ID NO: 448 to SEQ ID NO: 454. For example, in some instances, the effector protein does not include an amino acid sequence having 36.5% or more identity with SEQ ID NO: 451, and the effector protein comprises, distributed through the protein starting from the N-terminus, (i) a sequence having at least the threshold identity with SEQ ID NO: 452, (ii) a sequence having at least the threshold identity with SEQ ID NO: 450, (iii) a sequence having at least the threshold identity with SEQ ID NO: 454, (iv) a sequence having at least the threshold identity with SEQ ID NO: 449, (v) a sequence having at least the threshold identity with SEQ ID NO: 448, and (vi) a sequence having at least the threshold identity with SEQ ID NO: 453.

In some embodiments, an effector protein having an amino acid sequence of any one recited in TABLE 1 can have higher activity relative to a reference effector protein (e.g., an effector protein having a different amino acid sequence as recited in TABLE 1). For example, an effector protein can have increased modification activity and/or increased substrate binding activity (e.g., substrate selectivity, specificity, and/or affinity) relative to the reference effector protein. In some embodiments, compositions, systems, and methods described herein comprise a viral vector comprising a nucleic acid encoding an engineered protein having higher activity relative to a reference effector protein (e.g., an effector protein having a different amino acid sequence as recited in TABLE 1). In some embodiments, an effector protein as described herein has one or more activities that are at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over the reference effector protein.

In some embodiments, the effector protein does not comprise at least one of sequences recited in TABLE 1. In some embodiments, the effector protein does not comprise any one of sequences recited in TABLE 1. In some embodiments, the effector protein does not comprise SEQ ID NO: 112. In some embodiments, the effector protein comprises any one of sequences recited in TABLE 1 except SEQ ID NO: 112.

### Engineered Proteins

Polypeptides (e.g., effector proteins) described herein can be modified by altering one or more amino acids (e.g., deletion, insertion, or substitution). In some embodiments, the modified polypeptide (e.g., effector protein) has one or more alterations at one or more positions in a region of the polypeptide that comprises substrate binding activity, catalytic activity, and/or binding affinity for a substrate such as a target nucleic acid, an guide nucleic acid, or a guide nucleic acid-target nucleic acid heteroduplex. In some embodiments, the modified polypeptide (e.g., effector protein) may have different nucleic acid-cleaving activity relative to the unmodified polypeptide. Engineered proteins are not identical to a naturally-occurring protein. Engineered proteins described herein include modified polypeptides (e.g., effector proteins), wherein the modified polypeptides comprise amino acid sequences that are at least 65%, at least 70%, at least 75%, at least 80%, at least 85%, at least 90%, at least 95%, at least 97%, or at least 98%, at least 99%, or 100% identical to any one of sequences recited in TABLE 1. In some embodiments, compositions, systems, and methods described herein comprise viral vector comprising a nucleic acid encoding an engineered protein. In some embodiments, compositions, systems, and methods described herein comprise viral vector comprising a nucleic acid encoding a modified polypeptide (e.g., effector protein).

In some embodiments, a modified polypeptide (e.g., effector protein) may comprise one or more amino acid changes (e.g., deletion, insertion, or substitution) that reduces the nucleic acid-cleaving activity of the polypeptide relative to the unmodified polypeptide. For example, a modified polypeptide can have increased modification activity and/or increased substrate binding activity (e.g., substrate selectivity, specificity, and/or affinity) relative to the unmodified polypeptide. In some embodiments, a modified polypeptide (e.g., effector protein) has one or more activities that are at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over the unmodified polypeptide.

In some embodiments, a modified polypeptide (e.g., effector protein) has reduced modification activity (e.g., a catalytically defective effector protein) or no modification activity (e.g., a catalytically inactive effector protein). In some embodiments, the modified polypeptide (e.g., effector protein) has at least 90%, at least 80%, at least 70%, at least 30%, at least 40%, at least 30%, at least 20%, at least 10%, or 0% one or more activities relative to an unmodified polypeptide. Accordingly, a polypeptide (e.g., effector protein) as used herein encompasses a modified polypeptide that does not have nuclease activity.

In some embodiments, a modified polypeptide (e.g., effector protein) can include one or more mutations, including an insertion, deletion or substitution (e.g., conservative or non-conservative substitution). A modified polypeptide (e.g., effector protein), in some embodiments, includes at least one mutation relative to a reference protein (e.g., a naturally-occurring protein). In some embodiments, a modified polypeptide (e.g., effector protein) includes at least 1, at least 2, at least 3, at least 4, at least 5, at least 6, at least 7, at least 8, at least 9, at least 10, at least 15, at least 20, at least 25 or at least 30 mutations relative to a reference protein (e.g., a naturally-occurring protein). In some embodiments, a modified polypeptide (e.g., effector protein) includes no more than 10, 20, 30, 40, or 50 mutations relative to a reference protein (e.g., a naturally-occurring protein).

When describing a mutation that changes an amino acid residue or a nucleotide as described herein, such a change or changes can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions, and substitution of one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in a mutation of the amino acid sequence or function of encoded peptide, polypeptide or protein, also known as a silent mutation. Methods of mutating an amino acid residue or a nucleotide are well known. In some embodiments, a modified polypeptide (e.g., effector protein) comprises an amino acid sequence of any one of the sequences recited in TABLE 1, but the amino acid sequence comprises one or more mutations such that the amino acid sequence is at least 65%, at least 70%, at least 75%, at least 80%, at least 85%, at least 90%, at least 95%, at least 97%, or at least 98%, or at least 99% identical to any one of sequences recited in TABLE 1. In some embodiments, such one or more mutations comprises one or more deletions, one or more insertions, one or more substitutions, or combinations thereof.

In some embodiments, conservative substitution is a replacement of one amino acid for another such that the replacement takes place within a family of amino acids that are related in their side chains. Conversely, in some embodiments, non-conservative substitution can be a replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M). In some instances, an effector protein comprises a sequence that is at least 70%, at least 75%, at least 80%, at least 85%, at least 90%, at least 95%, or at least 98% identical to a sequence recited in Table 1, wherein not more than 1, 2, 3, 4, 5, 6, 7, 8, 9 or 10 amino acid differences between the sequence and the sequence recited in Table 1 are non-conservative amino acid differences (the remaining amino acid differences being conservative amino acid substitutions).

In some embodiments, the polypeptides (e.g., effector proteins) described comprise one or more mutations. In some embodiments, the one or more mutations result amino acid sequences that are at least 65%, at least 70%, at least 75%, at least 80%, at least 85%, at least 90%, at least 95%, at least 97%, or at least 98%, at least 99%, or 100% identical to any one of sequences recited in TABLE 1. In some embodiments, the one or more mutations comprise one or more conservative substitutions, one or more non-conservative substitutions, and combinations thereof. In some embodiments, the conservative substitution comprises substitution of one or more acidic (negatively charged) amino acids with different acidic amino acids, one or more basic (positively charged) amino acids with different basic amino acids, one or more non-polar (hydrophobic) amino acids with different non-polar amino acids, one or more uncharged polar amino acids with different uncharged polar amino acids, or combinations thereof. In some embodiments, the polypeptides (e.g., effector proteins) described herein comprise one or more conservative substitutions, wherein one or more positively charged amino acids (e.g., Lys (K), Arg (R), and His (H)) are substituted with different positively charged amino acids. In some embodiments, the one or more positively charged amino acids, Lys (K) and His (H), are substituted with Arg (R). In some embodiments, the substitution improves binding and/or cleavage activity of a modified polypeptide (e.g., effector protein).

In some embodiments, the one or more mutations are one or more non-conservative substitutions. For example, the polypeptides (e.g., effector proteins) described herein can comprise one or more non-conservative substitutions, wherein one or more non-positively charged amino acids are substituted with a positively charged amino acid (e.g., Lys (K), Arg (R), and His (H)). In some embodiments, the non-conservative substitutions comprise substitution of one or more of Glu (E), Leu (L), Gln (Q), Ser (S), Thr (T), Asp (D), and Asn (N) with Arg (R).

In some embodiments, a modified polypeptide (e.g., effector protein) comprises at least one, at least two, at least three, or at least four substitutions relative to SEQ ID NO: 57, wherein the substitutions are selected from the group consisting of T23R, A24R, G25R, L28R, K29R, P51R, N52R, F53R, Q54R, G55R, G56R, P57R, L125R, I126R, I127R, K128R, N129R, A130R, V131R, T316R, Q511R, D512R, Y513R, K514R, P515R, K516R, L517R, N540R, K541R, L542R, S543R, K544R, S545R, Y590R, K591R, P592R, K593R, K594R, E595R, N596R, A602R, I603R, H604R, K605R, A606R, L607R, T608R, G13R, F14R, K15R, L16R, I17R, N19R, H20R, S21R, N30R, E31R, G32R, E33R, E34R, A35R, C36R, K37R, K38R, F39R, V40R, E42R, N43R, E44R, S108R, E109R, H110R, G111R, L112R, D113R, T114R, V115R, P116R, Y117R, K118R, E119R, A120R, A121R, G122R, L123R, N124R, N132R, T133R, Y134R, K135R, G136R, V137R, Q138R, V139R, G179R, Y180R, L181R, L182R, Q183R, K184R, P185R, S186R, P187R, N188R, K189R, S190R, I191R, Y192R, C193R, Y194R, Q195R, S196R, V197R, S198R, P199R, K200R, P201R, F202R, I203R, T204R, S205R, K206R, Y207R, H208R, N209R, V210R, T23K, A24K, G25K, L28K, P51K, N52K, F53K, Q54K, G55K, G56K, P57K, L125K, I126K, I127K, N129K, A130K, V131K, T316K, Q511K, D512K, Y513K, P515K, L517K, N540K, L542K, S543K, S545K, R546K, Y590K, P592K, E595K, N596K, A602K, I603K, H604K, A606K, L607K, T608K, G13K, F14K, L16K, I17K, R18K, N19K, H20K, S21K, R22K, N30K, E31K, G32K, E33K, E34K, A35K, C36K, F39K, V40K, R41K, E42K, N43K, E44K, S108K, E109K, H110K, G111K, L112K, D113K, T114K, V115K, P116K, Y117K, E119K, A120K, A121K, G122K, L123K, N124K, N132K, T133K, Y134K, G136K, V137K, Q138K, V139K, G179K, Y180K, L181K, L182K, Q183K, P185K, S186K, P187K, N188K, S190K, I191K, Y192K, C193K, Y194K, Q195K, S196K, V197K, S198K, P199K, P201K, F202K, I203K, T204K, S205K, Y207K, H208K, N209K, V210K, T23H, A24H, G25H, L28H, K29H, P51H, N52H, F53H, Q54H, G55H, G56H, P57H, L125H, I126H, I127H, K128H, N129H, A130H, V131H, T316H, Q511H, D512H, Y513H, K514H, P515H, K516H, L517H, N540H, K541H, L542H, S543H, K544H, S545H, R546H, Y590H, K591H, P592H, K593H, K594H, E595H, N596H, A602H, I603H, K605H, A606H, L607H, T608H, G13H, F14H, K15H, L16H, I17H, R18H, N19H, S21H, R22H, N30H, E31H, G32H, E33H, E34H, A35H, C36H, K37H, K38H, F39H, V40H, R41H, E42H, N43H, E44H, S108H, E109H, G111H, L112H, D113H, T114H, V115H, P116H, Y117H, K118H, E119H, A120H, A121H, G122H, L123H, N124H, N132H, T133H, Y134H, K135H, G136H, V137H, Q138H, V139H, G179H, Y180H, L181H, L182H, Q183H, K184H, P185H, S186H, P187H, N188H, K189H, S190H, I191H, Y192H, C193H, Y194H, Q195H, S196H, V197H, S198H, P199H, K200H, P201H, F202H, I203H, T204H, S205H, K206H, Y207H, N209H, and V210H. In some embodiments, a modified polypeptide (e.g., effector protein) comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 57, wherein the modified polypeptide comprises: one or more conservative substitutions selected from H208R and K184R; one or more non-conservative substitutions selected from E109R, K38R, L182R, Q183R, S108R, S198R, T114R, and L26R; or combinations thereof. In some embodiments, a modified polypeptide (e.g., effector protein) comprises at least one substitution relative to SEQ ID NO: 57, wherein the substitution is selected from the group consisting of H208R, K184R, E109R, K38R, L182R, Q183R, S108R, S198R, Ti 14R, and L26R. In some embodiments, a modified polypeptide (e.g., effector protein) comprises at least two substitutions relative to SEQ ID NO: 57, wherein a first substitution is L26R and a second substitution is selected from the group consisting of H208R, K184R, E109R, K38R, L182R, Q183R, S108R, S198R, and T114R. In some embodiments, a modified polypeptide (e.g., effector protein) comprises at least two substitutions relative to SEQ ID NO: 57, wherein a first substitution is K184R and a second substitution is selected from the group consisting of H208R, L26R, E109R, K38R, L182R, Q183R, S108R, S198R, and T114R. In some embodiments, a modified polypeptide (e.g., effector protein) comprises at least three substitutions relative to SEQ ID NO: 57, wherein a first substitution is L26R, a second substitution is K184R, and a third substitution is H208R. In some embodiments, a modified polypeptide (e.g., effector protein) comprises at least three substitutions relative to SEQ ID NO: 57, wherein a first substitution is L26R, a second substitution is K184R, and a substitution is Q183R. In some embodiments, a modified polypeptide (e.g., effector protein) comprises at least three substitutions relative to SEQ ID NO: 57, wherein a first substitution is L26R, a second substitution is H208R, and a third substitution is Q183R. In some embodiments, a modified polypeptide (e.g., effector protein) comprises at least four substitutions relative to SEQ ID NO: 57, wherein a first substitution is L26R, a second substitution is Q183R, a third substitution is Q184R, and a substitution is T114R.

In some embodiments, a modified polypeptide (e.g., effector protein) comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 23, wherein the modified polypeptide comprises: one or more conservative substitutions selected from K261R and K466R; one or more non-conservative substitutions selected from S460R, D462R, N459R, N467R, T281R, E282R, T278R, T124R, T128R, N129R, E127R, and L126R; or combinations thereof. In some embodiments, a modified polypeptide (e.g., effector protein) comprises at least one substitution relative to SEQ ID NO: 23, wherein the substitution is selected from the group consisting of S460R, D462R, N459R, N467R, T281R, E282R, T278R, T124R, T128R, N129R, E127R, and L126R.

In some embodiments, the modified polypeptide (e.g., effector protein) has at least one amino acid residue alteration relative to a sequence of an unmodified polypeptide, wherein the at least one amino acid residue alteration is a conservative amino acid substitution. In some aspects, such a conservative amino acid sequence is a chemically conservative or an evolutionary conservative amino acid substitution. Methods of identifying conservative amino acids are well known to one of skill in the art, any one of which can be used to generate the effector proteins described herein. In some embodiments, the modified polypeptide (e.g., effector protein) has at least one amino acid residue alteration relative to a sequence of the polypeptide, wherein the at least one amino acid residue alteration is a non-conservative amino acid substitution.

In some embodiments, a modified polypeptide (e.g., effector protein) comprises one or more fusion partner. In some embodiments, the fusion partner protein comprises heterologous polypeptides (e.g., effector proteins). In certain embodiments, the fusion partner protein comprises a subcellular localization sequence. A subcellular localization signal may be located at or near the amino terminus (N-terminus) of the modified polypeptides (e.g., effector proteins) disclosed herein. A subcellular localization signal may be located at or near the carboxy terminus (C-terminus) of the modified polypeptides (e.g., effector proteins) disclosed herein. In some embodiments, the modified polypeptide (e.g., effector protein) described herein comprises: 1, 2, 3, 4, 5, 6, 7, 8, 9, 10, or more subcellular localization signals at or near the N-terminus; 1, 2, 3, 4, 5, 6, 7, 8, 9, 10, or more subcellular localization signals at or near the C-terminus; or combinations of these (e.g. one or more subcellular localization signals at the amino-terminus and one or more subcellular localization signals at the carboxy terminus). When more than one subcellular localization signal is present, each may be selected independently of the others, such that a single subcellular localization signal may be present in more than one copy and/or in combination with one or more other subcellular localization signals present in one or more copies. In some embodiments, a subcellular localization signal is considered near the N- or C-terminus when the nearest amino acid of the subcellular localization signal is within about 1, 2, 3, 4, 5, 10, 15, 20, 25, 30, 40, 50, or more amino acids along the polypeptide chain from the N- or C-terminus.

In some embodiments, the subcellular localization sequence can be a nuclear localization signal (NLS) for targeting to the nucleus, a sequence to keep the fusion protein out of the nucleus, e.g., a nuclear export sequence (NES), a sequence to keep a fusion protein retained in the cytoplasm, a mitochondrial localization signal for targeting to the mitochondria, a chloroplast localization signal for targeting to a chloroplast, an ER retention signal, and the like. In some embodiments, a nuclear localization signal (NLS) comprises an entity (e.g., peptide) that facilitates localization of a nucleic acid, protein, or small molecule to the nucleus, when present in a cell that contains a nuclear compartment.

In some embodiments, a vector encodes the polypeptides (e.g., effector proteins) described herein, wherein the vector or vector systems disclosed herein comprises one or more NLSs, such as about or more than about 1, 2, 3, 4, 5, 6, 7, 8, 9, 10, or more NLSs. When more than one NLS is present, each may be selected independently of the others, such that a single NLS may be present in more than one copy and/or in combination with one or more other NLSs present in one or more copies. In certain embodiments, an NLS described herein comprises an NLS sequence recited in TABLE 2.

TABLE 2
Subcellular Localization Sequence

| SEQ ID NO: | Description | Sequence* |
|---|---|---|
| 216 | NLS | KR(K/R)R |
| 217 | NLS | (P/R)XXKR(\hat{ }DE)(K/R) |
| 218 | NLS | KRX(W/F/Y)XXAF |
| 219 | NLS | (R/P)XXKR(K/R)(\hat{ }DE) |
| 220 | NLS | LGKR(K/R)(W/F/Y) |
| 221 | NLS | KRX₁₀K(K/R)(K/R) |
| 222 | NLS | APKKKRKVGIHGVPAA |
| 223 | NLS | KRPAATKKAGQAKKKKEF |
| 641 | NLS | K(K/R)RK |
| 642 | NLS | KRX₁₁K(K/R)(K/R) |
| 643 | NLS | KRX₁₂K(K/R)(K/R) |
| 644 | NLS | KRX₁₀K(K/R)X(K/R) |
| 645 | NLS | KRX₁₁K(K/R)X(K/R) |
| 646 | NLS | KRX₁₂K(K/R)X(K/R) |

*wherein X is any naturally occurring amino-acid;
and \hat{ }D/E is any amino-acid except Asp or Glu

In some embodiments, viral vectors described herein comprise a nucleotide sequence that encodes an effector protein, wherein the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences recited in TABLE 1, wherein the effector protein further comprises any one of NLS sequence recited in TABLE 2. In some embodiments, the effector protein recognizes a corresponding PAM sequence as identified in TABLE 1.1.

In some cases, effector proteins described herein can be modified with a tag (also referred to as a protein tag). A tag can be a heterologous polypeptide (e.g., effector protein) that is detectable for use in tracking and/or purification. Accordingly, in some embodiments, an effector protein, composition, system and methods described herein may comprise a purification tag and/or a fluorescent protein. Non-limiting examples of purification tags include a histidine tag, e.g., a 6XHis tag; a hemagglutinin (HA) tag; a FLAG tag; a Myc tag; and maltose binding protein (MBP). Non-limiting examples of fluorescent proteins include green fluorescent protein (GFP), yellow fluorescent protein (YFP), red fluorescent protein (RFP), cyan fluorescent protein (CFP), mCherry, and tdTomato.

In another example, polypeptides (e.g., effector proteins) provided herein may be codon optimized.

In some embodiments, polypeptides (e.g., effector proteins) described herein are encoded by a codon optimized nucleic acid. In some embodiments, a nucleic acid sequence encoding a polypeptide (e.g., effector protein) described herein, is codon optimized. This type of optimization can entail a mutation of a polypeptide (e.g., effector protein) encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized polypeptide-encoding nucleotide sequence could be used. As another non-limiting example, if the intended host cell were a mouse cell, then a mouse codon-optimized polypeptide-encoding nucleotide sequence could be generated. As another non-limiting example, if the intended host cell were a eukaryotic cell, then a eukaryote codon-optimized polypeptide nucleotide sequence could be generated. As another non-limiting example, if the intended host cell were a prokaryotic cell, then a prokaryote codon optimized polypeptide-encoding nucleotide sequence could be generated. Codon usage tables are readily available, for example, at the “Codon Usage Database” available at www.kazusa.or.jp/codon. Accordingly, in some embodiments, polypeptides (e.g., effector proteins) described herein may be codon optimized for expression in a specific cell, for example, a bacterial cell, a plant cell, a eukaryotic cell, an animal cell, a mammalian cell, or a human cell. In some embodiments, the polypeptide (e.g., effector protein) is codon optimized for a human cell.

It is understood that when describing coding sequences of polypeptides (e.g., effector proteins) described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a modifying heterologous peptide, such as a fusion protein partner, is located at the N terminus ofthe effector protein, a start codon for the fusion protein partner serves as a start codon for the effector protein as well. Thus, the natural start codon encoding an amino acid residue sufficient for initiating translation (e.g., Methionine (M) or a Valine (V)) of the effector protein may be removed or absent.

In some embodiments, effector proteins disclosed herein are engineered proteins. Accordingly, an effector protein, composition, system and methods described herein may comprise a nuclear localization signal (NLS). In some cases, an NLS comprises an entity (e.g., peptide) that facilitates localization of a nucleic acid, protein, or small molecule to the nucleus, when present in a cell that contains a nuclear compartment. An NLS can be located at or near the amino terminus (N-terminus) of the effector protein disclosed herein. An NLS can be located at or near the carboxy terminus (C-terminus) of the effector protein s disclosed herein. In some embodiments, an effector protein described herein comprises 1, 2, 3, 4, 5, 6, 7, 8, 9, 10, or more NLSs at or near the N-terminus, 1, 2, 3, 4, 5, 6, 7, 8, 9, 10, or more NLSs at or near the C-terminus, or a combination of these (e.g. one or more NLS at the amino-terminus and one or more NLS at the carboxy terminus). In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide (e.g., effector protein) chain from the N- or C-terminus. Accordingly, in some embodiments, effector proteins described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% identical to any one of the sequences as set forth in TABLE 1 and further comprises one or more sequence set forth in TABLE 2. Accordingly, in some embodiments, effector proteins described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% identical to any one ofthe sequences as set forth in TABLE 1 and further comprises one or more sequence set forth in TABLE 2.

In some instances, effector proteins disclosed herein are engineered proteins. Engineered proteins are not identical to a naturally occurring protein. Engineered proteins may provide enhanced nuclease or nickase activity as compared to a naturally occurring nuclease or nickase. An engineered protein may comprise a modified form of a wildtype counterpart protein.

In some instances, effector proteins comprise at least one amino acid change (e.g., deletion, insertion, or substitution) that reduces the nucleic acid-cleaving activity of the effector protein relative to the wildtype counterpart. For example, a nuclease domain (e.g., RuvC domain, HEPN domain) of an effector protein may be deleted or mutated relative to a wildtype counterpart effector protein so that it is no longer functional or comprises reduced nuclease activity. The effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins may have no substantial nucleic acid-cleaving activity. Engineered proteins may be enzymatically inactive or “dead,” that is it may bind to a guide nucleic acid and/or a target nucleic acid but not cleave the target nucleic acid. An enzymatically dead protein is also referred to in some instances as a dead protein or a dCas protein. An enzymatically inactive protein may comprise an enzymatically inactive domain (e.g. inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid sequence. In some instances, the enzymatically inactive protein is fused with a fusion partner protein that confers an alternative activity to an engineered protein activity. Such fusion proteins are described herein and throughout. In some embodiments, a fusion partner protein comprising an alternative activity includes but not limited to a transcriptional activation, transcription repression, deaminase activity, transposase activity, and recombinase activity.

In some instances, effector proteins comprise at least one amino acid change (e.g., deletion, insertion, or substitution) that increases the nucleic acid-cleaving activity of the effector protein relative to the wildtype counterpart. The effector protein may provide at least about 20%, at least about 30%, at least about 40%, at least about 50% at least about 60%, at least about 70%, at least about 80%, at least about 90%, or about 100% more nucleic acid-cleaving activity relative to that of the wild-type counterpart. The effector protein may provide at least about 2 fold, at least about 3 fold, at least about 4 fold, at least about 5 fold, at least about 6 fold, at least about 7 fold, at least about 8 fold, at least about 9 fold or at least about 10 fold more nucleic acid-cleaving activity relative to that of the wild-type counterpart.

In general, the viral vector encodes an mRNA that is translated into the effector protein. In some instances, the mRNA comprises a polyA tail. This may increase the stability of the effector protein mRNA, thereby increasing production of effector protein.

### Fusion Proteins

A person skilled in the art would understand that the terms “fusion effector protein,” “fusion protein,” and “fusion polypeptide” are interchangeable. In some embodiments, compositions, systems, and methods described herein comprise viral vectors comprising a nucleotide sequence encoding fusion proteins. In general, a fusion protein comprises at least two heterologous polypeptides. In some instances, an effector protein is a fusion protein, wherein the fusion protein comprises a effector protein and a fusion partner protein. A fusion partner protein is also simply referred to herein as a fusion partner. In general, a fusion partner protein is not an effector protein. The fusion partner may comprise a protein or a functional domain thereof. In some embodiments, the fusion partner protein is fused, or linked via a linker, to an effector protein. The fusion partner generally imparts some function to the fusion protein that is not provided by the effector protein. In some embodiments, the fusion partner provides a detectable signal. In some embodiments, the fusion partner modifies a target nucleic acid. In some embodiments, the fusion partner makes a chemical modification to one or more nucleotides of a target nucleic acid. By way of non-limiting example, a fusion partner may modify a nucleobase of the target nucleic acid to an alternative nucleobase. The fusion partner may be capable of modulating expression of a target nucleic acid. The fusion partner may inhibit, reduce, activate or increase expression of a target nucleic acid. The fusion partner may interact with additional proteins to make modifications to a target nucleic acid. Non-limiting examples of fusion partners include cell surface receptor proteins, intracellular signaling proteins, transcription factors, or functional domains thereof. The fusion partner may comprise a signaling peptide, e.g., a nuclear localization signal (NLS). In some embodiments, a nuclear localization signal is an entity (e.g., peptide) that facilitates localization of a nucleic acid, protein, or small molecule to the nucleus, when present in a cell that contains a nuclear compartment. In some cases, an effector protein described herein is not modified with an NLS so that the polypeptide (e.g., effector protein) is not targeted to the nucleus, which can be advantageous depending on the circumstance (e.g., when the target nucleic acid is an RNA that is present in the cytosol).

In some instances, the fusion partner modulates transcription (e.g., inhibits transcription, increases transcription) of a target nucleic acid. In some instances, the fusion partner is a protein (or a domain from a protein) that inhibits transcription of a target nucleic acid, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof. In some instances, the fusion partner is a protein (or a domain from a protein) that increases transcription of a target nucleic acid, also referred to as a transcription activator. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof.

In some instances, the fusion protein is a base editor. In general, a base editor comprises a deaminase. In some instances, a fusion protein that comprises a deaminase and an effector protein changes a nucleobase to a different nucleobase, e.g., cytosine to thymine or guanine to adenine.

In some embodiments, fusion proteins are targeted by a guide nucleic acid (e.g., guide RNA) to a specific location in the target nucleic acid and exert locus-specific regulation such as blocking RNA polymerase binding to a promoter (which selectively inhibits transcription activator function), and/or modifying the local chromatin status (e.g., when a fusion sequence is used that modifies the target nucleic acid or modifies a protein associated with the target nucleic acid). In some embodiments, the modifications are transient (e.g., transcription repression or activation). In some embodiments, the modifications are inheritable. For embodiment, epigenetic modifications made to a target nucleic acid, or to proteins associated with the target nucleic acid, e.g., nucleosomal histones, in a cell, are observed in cells produced by proliferation of the cell.

In some instances, fusion partners provide enzymatic activity that modifies a target nucleic acid. Such enzymatic activities include, but are not limited to, histone acetyltransferase activity, histone deacetylase activity, nuclease activity, methyltransferase activity, demethylase activity, DNA repair activity, DNA damage activity, deamination activity, dismutase activity, alkylation activity, depurination activity, oxidation activity, kinase activity, phosphatase activity, pyrimidine dimer forming activity, integrase activity, transposase activity, recombinase activity, polymerase activity, ligase activity, helicase activity, photolyase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity, and glycosylase activity. In some instances, the fusion partner comprises an RNA splicing factor.

In some embodiments, viral vectors as described herein comprise nucleotide sequences encoding fusion proteins. In some embodiments, an effector protein is a fusion protein, wherein the fusion protein comprises an effector protein and a fusion partner protein. In some embodiments, the effector protein comprises an amino acid sequence that is at least 70%, at least 75%, at least 80%, at least 85%, at least 90%, at least 95%, or 100% identical to any one of the sequences set forth in TABLE 1. In some embodiments, the amino acid of the effector protein is at least 70%, at least 75%, at least 80%, at least 85%, at least 90%, at least 95%, or 100% identical to any one of the sequences set forth in TABLE 1. Unless otherwise indicated, reference to effector proteins throughout the present disclosure include fusion proteins thereof.

In some embodiments, the fusion partner inhibits the formation of a multimeric complex of the effector protein. In some embodiments, the fusion partner promotes the formation of a multimeric complex of the effector protein. By way of non-limiting example, the fusion protein may comprise a fusion partner comprising a Calcineurin A tag, wherein the fusion protein dimerizes in the presence of Tacrolimus (FK506). Also by way of non-limiting example, the fusion protein may comprise a SpyTag configured to dimerize or associate with another effector protein in a multimeric complex.

### Modifying Target Nucleic Acids

In some embodiments, a modified target nucleic acid is a target nucleic acid that has undergone a modification, for example, after contact with an effector protein. In some embodiments, the modification is an alteration in the sequence of the target nucleic acid. In some embodiments, the modified target nucleic acid comprises an insertion, deletion, or replacement of one or more nucleotides compared to the unmodified target nucleic acid.

In some cases, fusion partners provide enzymatic activity that modifies a target nucleic acid. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity. In some cases, nuclease activity comprises the enzymatic activity of an enzyme which allows the enzyme to cleave the phosphodiester bonds between the nucleotide subunits of nucleic acids. In some case, an enzyme with nuclease activity can comprise a nuclease.

Disclosed herein are compositions and methods for modifying a target nucleic acid using viral vectors. The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions and methods reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions and methods increase expression of one or more genes by at least 10%, at least 20%, at least 30%, at least 40%, at least 50%, at least 60%, at least 70%, at least 80%, at least 90%, or at least 100%.

In some instances, compositions and methods use viral vectors encoding effector proteins that are fused to a heterologous protein. Heterologous proteins include, but are not limited to, transcriptional activators, transcriptional repressors, deaminases, methyltransferases, acetyltransferases, and other nucleic acid modifying proteins. In some cases, effector proteins need not be fused to a partner protein to accomplish the required protein (expression) modification.

In some cases, fusion partners have enzymatic activity that modifies the target nucleic acid. The target nucleic acid may comprise or consist of a ssRNA, dsRNA, ssDNA, or a dsDNA. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins. It is understood that a fusion protein may include the entire protein or in some cases may include a fragment of the protein (e.g., a functional domain). In some instances, the functional domain interacts with or binds ssRNA, including intramolecular and/or intermolecular secondary structures thereof, e.g., hairpins, stem-loops, etc.). The functional domain may interact transiently or irreversibly, directly or indirectly. In some cases, a functional domain comprises a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity. Fusion proteins may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for repressing translation (e.g., Ago2 and Ago4); proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); proteins and protein domains responsible for RNA localization (e.g., from IMP1, ZBP1, She2p, She3p, and Bicaudal-D); proteins and protein domains responsible for nuclear retention of RNA (e.g., Rrp6); proteins and protein domains responsible for nuclear export of RNA (e.g., TAP, NXF1, THO, TREX, REF, and Aly); proteins and protein domains responsible for repression of RNA splicing (e.g., PTB, Sam68, and hnRNP A1); proteins and protein domains responsible for stimulation of RNA splicing (e.g., Serine/Arginine-rich (SR) domains); proteins and protein domains responsible for reducing the efficiency of transcription (e.g., FUS (TLS)); and proteins and protein domains responsible for stimulating transcription (e.g., CDK7 and HIV Tat). Alternatively, the effector domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627, which is hereby incorporated by reference in its entirety.

In some instances, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

### Base Editors

In some embodiments, fusion partners modify a nucleobase of a target nucleic acid. Fusion proteins comprising such fusion partners and an effector protein may be referred to as base editors. When a base editor is described herein, it can refer to a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

In some embodiments, base editors modify a sequence of a target nucleic acid. In some embodiments, base editors provide a nucleobase change in a DNA molecule. In some embodiments, the nucleobase change in the DNA molecule is selected from: an adenine (A) to guanine (G); cytosine (C) to thymine (T); and cytosine (C) to guanine (G). In some embodiments, base editors provide a nucleobase change in an RNA molecule. In some embodiments, the nucleobase change in the RNA molecule is selected from: adenine (A) to guanine (G); uracil (U) to cytosine (C); cytosine (C) to guanine (G); and guanine (G) to adenine (A). In some embodiments, the fusion partner is a deaminase, e.g., ADAR½.

Some base editors modify a nucleobase of on a single strand of DNA. In some embodiments, base editors modify a nucleobase on both strands of dsDNA. In some embodiments, upon binding to its target locus in DNA, base pairing between the guide RNA and target DNA strand leads to displacement of a small segment of single-stranded DNA in an “R-loop”. In some embodiments, DNA bases within the R-loop are modified by the deaminase enzyme. In some embodiments, DNA base editors for improved efficiency in eukaryotic cells comprise a catalytically inactive effector protein that may generate a nick in the non-edited DNA strand, inducing repair of the non-edited strand using the edited strand as a template.

In some embodiments, a catalytically inactive effector protein can comprise an effector protein that is modified relative to a naturally-occurring nuclease to have a reduced or eliminated catalytic activity relative to that of the naturally-occurring nuclease, but retains its ability to interact with a guide nucleic acid. The catalytic activity that is reduced or eliminated is often a nuclease activity. The naturally-occurring nuclease may be a wildtype protein. In some embodiments, the catalytically inactive effector protein is referred to as a catalytically inactive variant of a nuclease/effector protein, e.g., a Cas nuclease/effector protein.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise viral vectors encoding a base editor and a guide nucleic acid, wherein the guide nucleic acid directs the base editor to a sequence in a target gene. The target gene may be associated with a disease. In some embodiments, the guide nucleic acid directs that base editor to or near a mutation in the sequence of a target gene. The mutation may be the deletion of one more nucleotides. The mutation may be the addition of one or more nucleotides. The mutation may be the substitution of one or more nucleotides. The mutation may be the insertion, deletion or substitution of a single nucleotide, also referred to as a point mutation. The point mutation may be a SNP. The mutation may be associated with a disease. In some embodiments, the guide nucleic acid directs the base editor to bind a target region within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary or reverse complementary to a target region of a target nucleic acid that comprises the mutation. In some embodiments, the guide nucleic acid comprises a sequence that is identical, complementary or reverse complementary to a target region of a target nucleic acid that is within 1, 2, 3, 4, 5, 6, 7, 8, 9, 10, 11, 12, 13, 14, 15, 16, 17, 18, 19, or 20 nucleotides of the mutation.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a viral vector encoding a base editor and a guide nucleic acid, wherein the guide nucleic acid directs the base editor to a sequence in a target gene

In some embodiments, fusion partners comprise a base editing enzyme. When a base editing enzyme is described herein, it can refer to a protein, polypeptide, or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

In some embodiments, the base editing enzyme modifies the nucleobase of a deoxyribonucleotide. In some embodiments, the base editing enzyme modifies the nucleobase of a ribonucleotide. A base editing enzyme that converts a cytosine to a guanine or thymine may be referred to as a cytosine base editing enzyme. A base editing enzyme that converts an adenine to a to a guanine may be referred to as an adenine base editing enzyme. In some embodiments, the base editing enzyme comprises a deaminase enzyme. In some embodiments, the deaminase functions as a monomer. In some embodiments, the deaminase functions as heterodimer with an additional protein. In some embodiments, base editors comprise a DNA glycosylase inhibitor. In some embodiments, base editors comprise a uracil glycosylase inhibitor (UGI) or uracil N-glycosylase (UNG). In some embodiments, base editors do not comprise a U•GI. In some embodiments, base editors do not comprise a UNG. In some embodiments, base editors do not comprise a functional fragment of a U•GI. A functional fragment of a U•GI is a fragment of a U•GI that is capable of excising a uracil residue from DNA by cleaving an N-glycosidic bond. In some cases, a functional fragment comprises a fragment of a protein that retains some function relative to the entire protein. Non-limiting examples of functions are nucleic acid binding, protein binding, nuclease activity, nickase activity, deaminase activity, demethylase activity, or acetylation activity. In some embodiments, a functional fragment may be a recognized functional domain, e.g., a catalytic domain such as, but not limited to, a RuvC domain.

In some embodiments, a base editing enzyme can comprise a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase. In some cases, a base editor can be a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

In some embodiments, the base editor is a cytidine deaminase base editor generated by ancestral sequence reconstruction as described in WO2019226953, which is hereby incorporated by reference in its entirety.

Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety.

In some embodiments, the base editor is a cytosine base editor (CBE). In general, a CBE comprises a cytosine base editing enzyme and a catalytically inactive effector protein. In some embodiments, the catalytically inactive effector protein is a catalytically inactive variant of an effector protein described herein. The CBE may convert a cytosine to a thymine. In some embodiments, the base editor is an adenine base editor (ABE). In general, an ABE comprises an adenine base editing enzyme and a catalytically inactive effector protein. In some embodiments, the catalytically inactive effector protein is a catalytically inactive variant of an effector protein described herein. The ABE generally converts an adenine to a guanine. In some embodiments, the base editor is a cytosine to guanine base editor (CGBE). In general, a CGBE converts a cytosine to a guanine.

In some embodiments, the base editor is a CBE. In some embodiments, the cytosine base editing enzyme is a cytidine deaminase. In some embodiments, the cytosine deaminase is an APOBEC1 cytosine deaminase, which accept ssDNA as a substrate but is incapable of cleaving dsDNA, fused to a catalytically inactive effector protein. In some embodiments, when bound to its cognate DNA, the catalytically inactive effector protein performs local denaturation of the DNA duplex to generate an R-loop in which the DNA strand not paired with the guide RNA exists as a disordered single-stranded bubble. In some embodiments, the catalytically inactive effector protein generated ssDNA R-loop enables the CBE to perform efficient and localized cytosine deamination in vitro. In some examples, deamination activity is exhibited in a window of about 4 to about 10 base pairs. In some embodiments, fusion to the catalytically inactive effector protein presents the target site to APOBEC1 in high effective molarity, enabling the CBE to deaminate cytosines located in a variety of different sequence motifs, with differing efficacies. In some embodiments, the CBE is capable of mediating RNA-programmed deamination of target cytosines in vitro. In some embodiments, the CBE is capable of mediating RNA-programmed deamination of target cytosines in vivo. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C•G-to-G•C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U•G in DNA is initiated by a UNG, which recognizes the U•G mismatch and cleaves the glyosidic bond between uracil and the deoxyribose backbone of DNA. In some embodiments, BER results in the reversion of the U•G intermediate created by the first CBE back to a C•G base pair. In some embodiments, UNG may be inhibited by fusion of uracil DNA glycosylase inhibitor (UGI), in some embodiments, a small protein from bacteriophage PBS, to the C-terminus of the CBE. In some embodiments, U•GI is a DNA mimic that potently inhibits both human and bacterial UNG. In some embodiments, a U•GI inhibitor is any protein or polypeptide that inhibits UNG. In some embodiments, the CBE mediates efficient base editing in bacterial cells and moderately efficient editing in mammalian cells, enabling conversion of a C•G base pair to a T•A base pair through a U•G intermediate. In some embodiments, the CBE is modified to increase base editing efficiency while editing more than one strand of DNA.

In some embodiments, the CBE nicks the non-edited DNA strand. In some embodiments, the non-edited DNA strand nicked by the CBE biases cellular repair of the U•G mismatch to favor a UA outcome, elevating base editing efficiency. In some embodiments, the APOBEC1-nickase-UGI fusion efficiently edits in mammalian cells, while minimizing frequency of non-target indels.

In some embodiments, the cytidine deaminase is selected from APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9 (A840H)-UGI), BE3-Gam, saBE3, saBE4-Gam, BE4, BE4-Gam, saBE4, or saBE4-Gam as described in WO2021163587, WO202108746, WO2021062227, and WO2020123887, which are incorporated herein by reference in their entirety.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the non-protein uracil-DNA glycosylase inhibitor (npUGI) is a small molecule derived from uracil. Examples of small molecule non-protein uracil-DNA glycosylase inhibitors, fusion proteins, and Cas-CRISPR systems comprising base editing activity are described in W0202108746, which is incorporated by reference in its entirety.

In some embodiments, the fusion partner is a deaminase, e.g., ADAR½, ADAR-2, or AID. In some embodiments, the base editor is an ABE. In some embodiments, the adenine base editing enzyme of the ABE is an adenosine deaminase. In some embodiments, the adenine base editing enzyme is selected from ABE8e, ABE8.20m, APOBEC3A, Anc APOBEC (a.k.a. AncBE4Max), and BtAPOBEC2. In some embodiments, the ABE base editor is an ABE7 base editor. In some embodiments, the deaminase or enzyme with deaminase activity is selected from ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, or ABE8.24d. In some embodiments, the adenine base editing enzyme is ABE8.1d. In some embodiments, the adenosine base editor is ABE9. Exemplary deaminases are described in US20210198330, WO2021041945, WO2021050571A1, and WO2020123887, all of which are incorporated herein by reference in their entirety. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871. Additional examples of deaminase domains are also described in WO2018027078 and WO2017070632, which are hereby incorporated by reference in their entirety.

In some embodiments, an ABE converts an A•T base pair to a G•C base pair. In some embodiments, the ABE converts a target A•T base pair to G•C in vivo. In some embodiments, the ABE converts a target A•T base pair to G•C in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. In some embodiments, an ABE comprises an engineered adenosine deaminase enzyme capable of acting on ssDNA.

In some embodiments, a base editor comprises an adenosine deaminase variant that differs from a naturally occurring deaminase. Relative to the naturally occurring deaminase, the adenosine deaminase variant may comprise a V82S alteration, a T166R alteration, or a combination thereof. In some embodiments, the adenosine deaminase variant comprises at least one of the following alterations relative to a naturally occurring adenosine deaminase: Y147T, Y147R, Q154S, Y123H, and Q154R., which are incorporated herein by reference in their entirety.

In some embodiments, a base editor comprises a deaminase dimer. In some embodiments, a base editor is a deaminase dimer further comprising a base editing enzyme and an adenine deaminase (e.g., TadA).

In some embodiments, the adenosine deaminase is a TadA monomer (e.g., Tad*7.10, TadA*8 or TadA*9). In some embodiments, the adenosine deaminase is a TadA*8 variant. Such a TadA*8 variant includes TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety. In some embodiments, a base editor is a deaminase dimer comprising a base editing enzyme fused to TadA via a linker.

In some embodiments, the amino terminus of the fusion partner protein is linked to the carboxy terminus of the effector protein via the linker. In some embodiments, the carboxy terminus of the fusion partner protein is linked to the amino terminus of the effector protein via the linker.

In some embodiments, the base editing enzyme is fused to TadA at the N-terminus. In some embodiments, the base editing enzyme is fused to TadA at the C-terminus. In some embodiments, the base editing enzyme is a deaminase dimer comprising an ABE. In some embodiments, the deaminase dimer comprises an adenosine deaminase. In some embodiments, the deaminase dimer comprises TadA fused to an adenine base editing enzyme selected from ABE8e, ABE8.20m, APOBEC3A, Anc APOBEC (a.k.a. AncBE4Max), and BtAPOBEC2. In some embodiments TadA is fused to ABE8e or a variant thereof. In some embodiments TadA is fused to ABE8e or a variant thereof at the amino-terminus (ABE8e-TadA). In some embodiments, TadA is fused to ABE8e or a variant thereof at the carboxy terminus (ABE8e-TadA).

### Prime Editing

In some embodiments, a fusion protein and/or a fusion partner can comprise a prime editing enzyme. When used herein, a prime editing enzyme can describe a protein, polypeptide, or fragment thereof that is capable of catalyzing the modification (insertion, deletion, or base-to-base conversion) of a target nucleotide or nucleotide sequence in a nucleic acid. A prime editing enzyme capable of catalyzing such a reaction includes a reverse transcriptase. In some embodiments, such a prime editing enzyme is an M-MLV RT enzyme or a mutant thereof. In some embodiments, the M-MLV RT enzyme comprises at least one mutation selected from D200N, L603W, T330P, T306K, and W313F relative to wildtype M-MLV RT enzyme. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification.

In some embodiments, the target nucleic acid is a dsDNA molecule. In some embodiments, the pegRNA comprises a guide RNA comprising a first region that is bound by the effector protein, and a second region comprising a spacer sequence that is complementary to a target sequence of the target dsDNA molecule; a template RNA comprising a primer binding sequence that hybridizes to a primer sequence of the target dsDNA molecule that is formed when target nucleic acid is cleaved, and a template sequence that is complementary to at least a portion of the target sequence of the target dsDNA molecule with the exception of at least one nucleotide. In some embodiments, the spacer sequence is complementary to the target sequence on the target strand of the dsDNA molecule. In some embodiments, the spacer sequence is complementary to the target sequence on the non-target strand of the dsDNA molecule. In some instances, the primer binding sequence hybridizes to a primer sequence on the non-target strand of the target dsDNA molecule. In some instances, the primer binding sequence hybridizes to a primer sequence on the target strand of the target dsDNA molecule. In some instances, the target strand is cleaved.

In some instances, the non-target strand is cleaved.

### Recombinases

In some embodiments, the fusion partners comprise a recombinase domain. In some embodiments, the enzymatically inactive protein is fused with a recombinase. In some embodiments, the recombinase is a site-specific recombinase. In some embodiments, the fusion partners comprise a recombinase domain wherein the recombinase is a site-specific recombinase. In some embodiments, described herein is a programmed nuclease comprising reduced nuclease activity or no nuclease activity and fused with a recombinase, wherein the recombinase can be a site-specific recombinase. Such polypeptides can be used for site-directed transgene insertion. Examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include, but are not limited to, gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include, but are not limited to: Bxb1, wBeta, BL3, phiR4, A118, TG1, MR11, phi370, SPBc, TP901-1, phiRV, FC1, K38, phiBTI, and phiC31. Further discussion and examples of suitable recombinase fusion partners are described in U.S. Pat. No. 10,975,392, which is incorporated herein by reference in its entirety.

In some embodiments, the fusion protein comprises a linker that links the recombinase domain to the Cas-CRISPR domain of the effector protein. In some embodiments, the linker is The-Ser.

### Modifying Proteins

In some cases, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

### CRISPRa Fusions and CRISPRi Fusions

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some embodiments, fusion partners activate or increase expression of a target nucleic acid.

Fusion proteins comprising such fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. In some embodiments, a transcriptional activator can describe a polypeptide or a fragment thereof that can activate or increase transcription of a target nucleic acid molecule. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof.

Non-limiting examples of fusion partners that promote or increase transcription include, but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof.

In some embodiments, fusions partners inhibit or reduce expression of a target nucleic acid. Fusion proteins comprising such fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, a transcriptional repressor can describe a polypeptide or a fragment thereof that is capable of arresting, preventing, or reducing transcription of a target nucleic acid. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include, but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof.

### Additional Fusion Partners

In some cases, the fusion partner is a chloroplast transit peptide (CTP), also referred to as a plastid transit peptide. In some instances, this targets the fusion protein to a chloroplast. Chromosomal transgenes from bacterial sources must have a sequence encoding a CTP sequence fused to a sequence encoding an expressed protein if the expressed protein is to be compartmentalized in the plant plastid (e.g. chloroplast). The CTP is removed in a processing step during translocation into the plastid. Accordingly, localization of an exogenous protein to a chloroplast is often accomplished by means of operably linking a polynucleotide sequence encoding a CTP sequence to the 5′ region of a polynucleotide encoding the exogenous protein. In some cases, the CTP is located at the N-terminus of the fusion protein. Processing efficiency may, however, be affected by the amino acid sequence of the CTP and nearby sequences at the amino terminus (NH2 terminus) of the peptide.

In some cases, the fusion partner is an endosomal escape peptide. In some cases, an endosomal escape protein comprises the amino acid sequence GLFXALLXLLXSLWXLLLXA (SEQ ID NO: 455), wherein each X is independently selected from lysine, histidine, and arginine. In some cases, an endosomal escape protein comprises the amino acid sequence GLFHALLHLLHSLWHLLLHA (SEQ ID NO: 456). In some cases, the amino acid sequence of the endosomal escape protein is GLFXALLXLLXSLWXLLLXA (SEQ ID NO: 455) or GLFHALLHLLHSLWHLLLHA (SEQ ID NO: 456).

Further suitable fusion partners include, but are not limited to, proteins (or fragments/domains thereof) that are boundary elements (e.g., CTCF), proteins and fragments thereof that provide periphery recruitment (e.g., Lamin A, Lamin B, etc.), protein docking elements (e.g., FKBP/FRB, Pil1/Aby1, etc.).

### Linkers for Fusion Partners

In some embodiments, viral vectors described herein comprise fusion proteins comprising an effector protein and a fusion partner protein. The effector protein may be fused or linked to the fusion partner protein. The terms “fused” and “linked” may be used interchangeably. In some instances, the effector protein and the fusion partner are directly linked via a covalent bond. In general, effector proteins and fusion partners of a fusion effector protein are connected via a linker. The linker may comprise or consist of a covalent bond. The linker may comprise or consist of a chemical group. In some embodiments, the linker comprises an amino acid. In some cases, a linker comprises a bond or molecule that links a first polypeptide (e.g., first effector protein) to a second polypeptide (e.g., second effector protein). In some instances, a peptide linker comprises at least two amino acids linked by an amide bond. In general, the linker connects a terminus of the effector protein to a terminus ofthe fusion partner. In some embodiments, the carboxy terminus of the effector protein is linked to the amino terminus of the fusion partner. In some embodiments, the carboxy terminus of the fusion partner is linked to the amino terminus of the effector protein.

In some cases, a terminus ofthe effector protein is linked to a terminus ofthe fusion partner through an amide bond. In some embodiments, a terminus of the effector protein is linked to a terminus of the fusion partner through a peptide bond. In some instances, linkers comprise an amino acid. In some embodiments, linkers comprise a peptide. In some cases, an effector protein is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, when a linked amino acids is described herein, it can refer to at least two amino acids linked by an amide bond.

These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G)n, glycine-serine polymers (including, for example, (GS)n, GSGGSn, GGSGGSn, and GGGSn, where n is an integer of at least one), glycine-alanine polymers, and alanine-serine polymers. Exemplary linkers may comprise amino acid sequences including, but not limited to, GGSG, GGSGG, GSGSG, GSGGG, GGGSG, and GSSSG.

### Multimeric Complexes

In some instances, compositions, systems, and methods of the present disclosure comprise a vector that encodes an effector protein, wherein the effector protein forms a multimeric complex with another protein. In general, a multimeric complex comprises multiple proteins that non-covalently interact with one another. In some instances, the multimeric complex comprises a first effector protein encoded by the vector and a second effector protein encoded by the vector, wherein the first effector protein and the second effector protein are the same. In some instances, the multimeric complex comprises a first effector protein encoded by the vector and a second effector protein encoded by the vector, wherein the first effector protein and the second effector protein are the different. A multimeric complex may comprise enhanced activity relative to the activity of any one of its effector proteins alone. For example, a multimeric complex comprising two effector proteins may comprise greater nucleic acid binding affinity, cis-cleavage activity, and/or transcollateral cleavage activity than that of either of the effector proteins provided in monomeric form. A multimeric complex may have an affinity for a target region of a target nucleic acid and is capable of catalytic activity (e.g., cleaving, nicking or modifying the nucleic acid) at or near the target region. Multimeric complexes may be activated when complexed with a guide nucleic acid. Multimeric complexes may be activated when complexed with a guide nucleic acid and a target nucleic acid. In some instances, the multimeric complex cleaves the target nucleic acid. In some instances, the multimeric complex nicks the target nucleic acid.

Various aspects of the present disclosure include compositions, systems, and methods comprising viral vector encoding multiple effector proteins, and uses thereof, respectively. An effector protein comprising at least 65% sequence identity to any one ofthe sequences of TABLE 1 may be provided with a second effector protein. Two effector proteins may target different nucleic acid sequences. Two effector proteins may target different types of nucleic acids (e.g., a first effector protein may target double- and single-stranded nucleic acids, and a second effector protein may only target single-stranded nucleic acids).

In some embodiments, multimeric complexes comprise at least one effector protein, or a fusion protein thereof, comprising an amino acid sequence with at least 65%, at least 70%, at least 75%, at least 80%, at least 85%, at least 90%, at least 95% or 100% identity to any one of the sequences of TABLE 1. In some embodiments, multimeric complexes comprise at least one effector protein or a fusion protein thereof, wherein the amino acid sequence of the effector protein is at least 65%, at least 70%, at least 75%, at least 80%, at least 85%, at least 90%, at least 95% or 100% identical to any one of the sequences of TABLE 1.

In some embodiments, the multimeric complex is a dimer comprising two effector proteins of identical amino acid sequences. In some embodiments, the multimeric complex comprises a first effector protein and a second effector protein, wherein the amino acid sequence of the first effector protein is at least 90%, at least 92%, at least 94%, at least 96%, at least 98% identical, or at least 99% identical to the amino acid sequence of the second effector protein.

In some embodiments, the multimeric complex is a heterodimeric complex comprising at least two effector proteins of different amino acid sequences. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein.

In some embodiments, a multimeric complex comprises at least two effector proteins. In some embodiments, a multimeric complex comprises more than two effector proteins. In some embodiments, a multimeric complex comprises two, three or four effector proteins. In some embodiments, at least one effector protein of the multimeric complex comprises an amino acid sequence with at least 65%, at least 70%, at least 75%, at least 80%, at least 85%, at least 90%, at least 95% or 100% identity to any one of the sequences of TABLE 1. In some embodiments, each effector protein of the multimeric complex comprises an amino acid sequence with at least 65%, at least 70%, at least 75%, at least 80%, at least 85%, at least 90%, at least 95% or 100% identity to any one of the sequences of TABLE 1.

### V. Engineered Guide Nucleic Acids

The compositions, systems, and methods of the present disclosure may comprise a viral vector comprising a guide nucleic acid, DNA encoding a guide nucleic acid, or a use thereof. Unless otherwise indicated, compositions, systems, and methods comprising a guide nucleic acid or uses thereof, as described herein and throughout, include DNA molecules, such as a viral vector, that encode a guide nucleic acid. In some embodiments, the guide nucleic acid imparts activity or sequence selectivity to the effector protein.

Provided herein are compositions comprising a viral vector that encodes one or more guide nucleic acids. Guide nucleic acids may comprise DNA, RNA, or a combination thereof (e.g., RNA with a thymine base). Guide nucleic acids may include a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides.

Guide nucleic acids are often referred to as a “guide RNA.” The term “guide RNA,” as well as crRNA and sgRNA, includes guide nucleic acids comprising DNA bases, RNA bases and modified nucleobases. In general, a guide nucleic acid is a nucleic acid molecule that binds to an effector protein (e.g., a Cas effector protein), thereby forming a ribonucleoprotein complex (RNP). In some embodiments, when a guide nucleic acid and an effector protein form an RNP, at least a portion of the RNP binds, recognizes, and/or hybridizes to a target nucleic acid. Those skilled in the art will understand that in some embodiments, a RNP can hybridize to one or more target sequences in a target nucleic acid, thereby allowing the RNP to modify and/or recognize a target nucleic acid or sequence contained therein or to modify and/or recognize non-target sequences depending on the guide nucleic acid, and in some embodiments, the effector protein, used.

Guide nucleic acids, when complexed with an effector protein, may bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as the nuclease activity of an effector protein.

A guide nucleic acid may comprise a naturally occurring guide nucleic acid. A guide nucleic acid may comprise a non-naturally occurring guide nucleic acid, wherein the sequence of the guide nucleic acid, or any portion thereof, may be different from the sequence of a naturally occurring nucleic acid. In some embodiments, a non-naturally occurring guide nucleic acid includes a guide nucleic acid that is designed to contain a chemical or biochemical modification. In some embodiments, an effector protein or a multimeric complex thereof cleaves a precursor RNA (“pre-crRNA”) to produce a guide RNA, also referred to as a “mature guide RNA.” An effector protein that cleaves pre-crRNA to produce a mature guide RNA is said to have pre-crRNA processing activity. In some embodiments, a repeat sequence of a guide RNA comprises mutations or truncations relative to respective regions in a corresponding pre-crRNA.

In some cases, the guide comprises 10, 11, 12, 13, 14, 15, 16, 17, 18, 19, 20, 21, 22, 23, 24, 25, 26, 27, 28, 29, or 30 linked nucleosides. In general, a guide nucleic acid comprises at least linked nucleosides. In some instances, a guide nucleic acid comprises at least 25 linked nucleosides. A guide nucleic acid may comprise 10 to 50 linked nucleosides. In some cases, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some cases, the guide nucleic acid has about 10 to about 60, about 20 to about 50, or about 30 to about 40 linked nucleosides.

In some embodiments, the guide nucleic acid comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides that are complementary to a eukaryotic sequence. Such a eukaryotic sequence is a sequence of nucleotides that is present in a host eukaryotic cell. Such a sequence of nucleotides is distinguished from nucleotide sequences present in other host cells, such as prokaryotic cells, or viruses. Said sequences present in a eukaryotic cell can be located a gene, an exon, an intron, a non-coding (e.g., promoter or enhancer) region, a selectable marker, tag, signal, and the like.

In some embodiments, the guide nucleic acid may comprise a first region complementary to a target nucleic acid (FR1) and a second region that is not complementary to the target nucleic acid (FR2). In some cases, FR1 is located 5′ to FR2 (FR1-FR2). In some cases, FR2 is located 5′ to FR1 (FR2-FR1). In some embodiments, the first region can comprise or include a repeat region that interacts with the effector protein. In some embodiments, the second region can comprise or include a spacer region, wherein the spacer region can interact in a sequence-specific manner with (e.g., has complementarity with, or can hybridize to) a target nucleic acid.

In some embodiments, the guide nucleic acid or a nucleic acid encoding the guide nucleic acid comprises a spacer sequence and/or a repeat sequence. In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, the compositions, systems, and methods of the present disclosure may comprise viral vectors encoding an additional guide nucleic acid, or a use thereof. In some embodiments, the viral vectors encode at least two, at least three, at least four, at least five, at least six, at least seven, at least eight, at least nine, or at least ten additional guide nucleic acids. An additional guide nucleic acid can target an effector protein to a different location in the target nucleic acid by binding to a different portion of the target nucleic acid from the first guide nucleic acid. For example, a guide nucleic acid can bind a portion of the target nucleic acid that is upstream or downstream of the target gene in a cell or subject as described herein, wherein the additional guide nucleic acid can bind to a portion of the target nucleic acid that is located either upstream or downstream of where the first guide nucleic acid has targeted.

In such embodiments, the dual-guided compositions, systems, and methods described herein can modify the target nucleic acid in two locations. In some embodiments, a first guide nucleic acid may bind or cleave a first portion of a target nucleic acid and a second guide nucleic acid may bind or cleave a second portion of the target nucleic acid. The first portion and the second portion of the target nucleic acid may be located at least 10, at least 20, at least 30, at least 40, at least 50, at least 60, at least 70, at least 80, at least 90 or at least 100 nucleotides apart. The first portion and the second portion of the target nucleic acid may be located between 100 and 200, 200 and 300, 300 and 400, 400 and 500, 500 and 600, 600 and 700, 700 and 800, 800 and 900 or 900 and 1000 nucleotides apart. In some instances, the first portion and/or the second portion of the target nucleic acid are located in an intron of a gene. In some instances, the first portion and/or the second portion of the target nucleic acid are located in an exon of a gene. In some instances, the first portion and/or the second portion of the target nucleic acid span and exon-intron junction of a gene.

In some embodiments, the dual-guided compositions, systems, and methods described herein can cleave the target nucleic acid in the two locations targeted by the guide nucleic acids. In some embodiments, a donor nucleic acid is inserted in replacement of the deleted sequence. For example, in some embodiments, the first portion and/or the second portion of the target nucleic acid are located on either side of an exon and cutting at both sites results in deletion of the exon. The modification of the target nucleic acid at two different loci is referred to herein as “dual-cutting”. Accordingly, in some embodiments, dual-guide nucleic acid compositions, systems, and methods can comprise viral vectors encoding two effector proteins, individually corresponding a guide nucleic acid or a single effector protein with two different guide nucleic acid to achieve dual-cutting.

### Repeat Sequence

In some embodiments, the compositions, systems and methods described herein, including an AAV vector, comprise a guide nucleic acid, or a nucleic acid encoding the guide nucleic acid, comprising a portion of, or all of a repeat sequence that interacts with the effector protein. The repeat sequence may also be referred to as a “protein-binding segment.” Typically, the repeat sequence is adjacent to the spacer sequence. For example, a guide nucleic acid that interacts with the effector protein comprises a repeat sequence that is 5′ of the spacer sequence. In some embodiments, the repeat region is followed by the spacer region in the 5′ to 3′ direction. In some embodiments, the spacer region is followed by the repeat region in the 5′ to 3′ direction.

In some embodiments, the guide nucleic acid comprises more than one repeat sequences. In certain embodiments, the guide nucleic acid comprises a first repeat sequence, followed by a spacer sequence, and a second repeat sequence in the 5′ to 3′ direction. In some embodiments, the first and second repeat sequence are identical. In certain embodiments, the first and second repeat sequences are not identical.

In some embodiments, the spacer sequence and the direct repeat sequence(s) of the guide nucleic acid are present within the same molecule. In some embodiments, the spacer and repeat sequences are linked directly to one another. In some embodiments, a linker is present between the spacer and repeat sequences. The linker may comprise 1, 2, 3, 4, 5, 6, 7, 8, 9 or 10 nucleotides. The linker may comprise more than 10 nucleotides. In some instances, the linker comprises the sequence 5′-GAAA-3.′ In some embodiments, the spacer sequence and the repeat sequence(s) of the guide nucleic acid are present in separate molecules, which are joined to one another by base pairing interactions.

In some embodiments, the repeat region is between 10 and 50, 12 and 48, 14 and 46, 16 and 44, and 18 and 42 nucleotides in length. In certain embodiments, the repeat region is between 19 and 37 nucleotides in length. TABLE 3 shows exemplary repeat sequences.

TABLE 3
Exemplary Repeat Sequences

| Effector Protein | |
|---|---|
| SEQ ID NO: | Repeat Sequences (SEQ ID NO:) |
| 1 | CGUUGCAGCUCGCACGUUGGCACUGGUUGAAGG (SEQ ID NO: 481) |
| 1 | CGUUGCAGCUCGCACGUUGGCACUGGGUUGAAGG (SEQ ID NO: 482) |
| 1 | UUGGCACUGGUUGAAGG (SEQ ID NO: 483) |
| 1 | CACUGGUUGAAGG (SEQ ID NO: 484) |
| 2 | GUUGCAACUCACGCGCGUAUGUGGCUUGAAGG (SEQ ID NO: 485) |
| 3 | GUUGCAAUUCAUAUCUCCGGGUGGAUUGAAGG (SEQ ID NO: 486) |
| 4, 10 | GUUGCAGCGUGCGCGAGCGUGUGGCUUGAAGG (SEQ ID NO: 487) |
| 4 | AGCGUGUGGCUUGAAGG (SEQ ID NO: 488) |
| 4 | UGUGGCUUGAAGG (SEQ ID NO: 489) |
| 5 | GUUGCAACUCGCACGUGAAUGCGACUUGAAGG (SEQ ID NO: 490) |
| 5 | UGAAUGCGACUUGAAGG (SEQ ID NO: 491) |
| 6 | GAUGCAACUCGUGUGUAUGUGCGAGUUGAAGG (SEQ ID NO: 492) |
| 7 | GACGCAACUCGCGCGCGGGCAUGUAUUGAGGG (SEQ ID NO: 493) |
| 8 | GAUGCAUCUGACACAGCUGGGUGAGUUGAAGG (SEQ ID NO: 494) |
| 8 | GCUGGGUGAGUUGAAGG (SEQ ID NO: 495) |
| 9 | GUUGCAACACAUGUAUGUGGGUGAGUUGAAGG (SEQ ID NO: 496) |
| 11 | GUUGCAAUUUGUAUACGAGUGUGACUUGAAGG (SEQ ID NO: 497) |
| 12 | GCUGCAACACGCGCGGGUACGCGGGUUGAAGG (SEQ ID NO: 498) |
| 13 | GUUGCAACUCGCACGUUGGCACUGAUUGAAGG (SEQ ID NO: 499) |
| 14 | GCUGUAGCCCUGCUCAAAUUGUAGGGCGCAUGCAGG (SEQ ID NO: 500) |
| 14, 15, 16 | GUUGUAGUCGACCUGAAUCUGUGGGGUGCUUACAGG (SEQ ID NO: 501) |
| 14, 16, 19 | UGUGGGGUGCUUACAGG (SEQ ID NO: 502) |
| 16 | GGUGUAUGUAACCGCAAUUUGAAGGGUGCAUACAGG (SEQ ID NO: 503) |
| 17,20 | GUUGGAAUCGACCUUAAUUUGAGGUGUGCUUACAGG (SEQ ID NO: 504) |
| 18 | GCUGAAAGAGCAGAGAAUUUGUUGUGUGCAUACAGG (SEQ ID NO: 505) |
| 19 | GUUGGAGUCGGCUUGAAUCUGCGGGGUGCUUACAGG (SEQ ID NO: 506) |
| 21 | GUUGUAAGAGACCCGAAUUUUAGCUGUGUAUACAGG (SEQ ID NO: 639) |
| 22 | GUUGUGAAUGCAGGCAUUUUUGAUGGUAAAUCCAAC (SEQ ID NO: 640) |
| 22-26, 28-34, | UGGUACAUCCAAC (SEQ ID NO: 215) |
| 405, 410-411, | |
| 420, 422, | |
| 425, 432, | |
| 439-441 | |
| 23 | ACUGUCAGACAAUGCAAAAUGUGUGGUACAUCCAAC (SEQ ID NO: 508) |
| 23 | UGGUACAUCC (SEQ ID NO: 509) |
| 23 | UGGGGCAGUUGGUUGCCCUUAGCCUGAGGCAUUUAUUGCACUCGGGAA |
| | GUACCAUUACAUCCAAC (SEQ ID NO: 510) |
| 23 | UGGUACAUCCAACUCUAGGCGCC (SEQ ID NO: 511) |
| 23 | AAUGGUACAUCCAAC (SEQ ID NO: 512) |
| 23 | UGGUACAUCCAACUCUAGGC (SEQ ID NO: 513) |
| 23 | UGGUACAUCCAACUCUAGGCGC (SEQ ID NO: 514) |
| 23 | UGGUACAUCCAACUCUAGGCG (SEQ ID NO: 515) |
| 23 | UGGUACAUCCAACUCUAGG (SEQ ID NO: 516) |
| 23 | AAAUGGUACAUCCAAC (SEQ ID NO: 517) |
| 23 | UGGUACAUCCAACUCU (SEQ ID NO: 518) |
| 23 | UGGUACAUCCAACUC (SEQ ID NO: 519) |
| 23 | UGGUACAUCCAACU (SEQ ID NO: 520) |
| 23 | UGGUACAUCCAACUCUAG (SEQ ID NO: 521) |
| 23 | UGGUAUAUCCAAC (SEQ ID NO: 522) |
| 23 | UGGUACAUCCAACUCUA (SEQ ID NO: 523) |
| 23 | AUGGUACAUCCAAC (SEQ ID NO: 524) |
| 23 | UGGUACAUCCAA (SEQ ID NO: 525) |
| 23 | UGGUACAUCCA (SEQ ID NO: 526) |
| 24, 34, 106 | GCUGUCAGUAGUAGUAAAAAUGGGGGUACAUCCAAC (SEQ ID NO: 527) |
| 25, 31 | ACUGUCAGUACAUGCAAAAAUGAGGGUACAUCCAAC (SEQ ID NO: 528) |
| 26 | ACUGUCAGACAAUGCAAAAUGAGUGGUACAUCCAAC (SEQ ID NO: 529) |
| 27 | GCUGUUAGAACAUACAAAAUGAAAGGUACAUCCAAC (SEQ ID NO: 530) |
| 28 | GCUGCAUGUCAUGGCAAAAGGAAAGGUACAUCCAAC (SEQ ID NO: 531) |
| 29 | GCUGUCAGACACCUAAAAAAUGAGGGUACAUCCAAC (SEQ ID NO: 532) |
| 30, 32 | GCUGUGAGUCACAGUAAAAAUGAAGGUAUAUCCAAC (SEQ ID NO: 533) |
| 33 | GAUGUAUAUGCUAUGAUUUUGUAUGGUACAUCCAAC (SEQ ID NO: 534) |
| 34, 105, 107 | GUUGCAGAACCCGAAUAGACGAAUGAAGGAAUGCAAC (SEQ ID NO: 535) |
| 35 | GUUGAGCUCUGCAUUACGCAGAUGAAUGACGAG (SEQ ID NO: 536) |
| 35-36, 38-39, | GAUAUAUCUUGUAUGCAUAUGUAGGUUGUGAG (SEQ ID NO: 537) |
| 41-44 | |
| 35-36, 38, | GUUGCAACUUACGCAUAGGUGUAAAAUACGAGG (SEQ ID NO: 538) |
| 40-43, 413 | |
| 36 | GAUGCAACUUAGAUGCAUAUGUAAGUUGUGAG (SEQ ID NO: 539) |
| 36-38, 41-43, 45 | GUUGCAAUGAACGUAUGUGCAUGAGGUGUGAG (SEQ ID NO: 540) |
| 36, 38, 42-43, 437 | GUUGCAAUUCGUAUGCGCAGGUAAGUUUCGAG (SEQ ID NO: 541) |
| 36-38, 42-43, 45 | UGUGCAUGAGGUGUGAG (SEQ ID NO: 542) |
| 37 | GUUGCAAUCUGCGUACAGGCGUAAGAUGUGAG (SEQ ID NO: 543) |
| 37 | CAGGCGUAAGAUGUGAG (SEQ ID NO: 544) |
| 38, 43 | GAUCAUAUCUGCUUGUAUGGGUAUGCUGCGAG (SEQ ID NO: 545) |
| 38 | UAUGGGUAUGCUGCGAG (SEQ ID NO: 546) |
| 39, 41 | GUUGCAACUUACGCAUAGGUGUAAAAUACGAG (SEQ ID NO: 547) |
| 40 | GAUUAUAUCUGCUUGUAUGGGUAUACUGCGAG (SEQ ID NO: 548) |
| 42 | UCAGCUCACAACCUACAUAUGCAUACAAGAUAUAUCGU (SEQ ID NO: 549) |
| 44 | CAUAUGUAGGUUGUGAG (SEQ ID NO: 550) |
| 44 | UGUAGGUUGUGAG (SEQ ID NO: 551) |
| 45 | CAUGAGGUGUGAG (SEQ ID NO: 552) |
| 46 | GGAGAGAUCUCAAACGAUUGCUCGAUUAGUCGAGAC (SEQ ID NO: 553) |
| 47 | GUCGGAACGCUCAACGAUUGCCCCUCACGAGGGGAC (SEQ ID NO: 554) |
| 49, 63 | ACCAAAACGACUAUUGAUUGCCCAGUACGCUGGGAC (SEQ ID NO: 555) |
| 52, 59 | GGAUCCAAUCCUUUUUGAUUGCCCAAUUCGUUGGGAC (SEQ ID NO: 556) |
| 55 | GGAUCUGAGGAUCAUUAUUGCUCGUUACGACGAGAC (SEQ ID NO: 557) |
| 56, 61 | GUCUCAGCGUACUGAGCAAUCAAAAGGUUUCGCAGG (SEQ ID NO: 558) |
| 57, 60 | CUUUCAAGACUAAUAGAUUGCUCCUUACGAGGAGAC (SEQ ID NO: 559) |
| 57, 60 | AUUGCUCCUUACGAGGAGAC (SEQ ID NO: 233) |
| 58 | GUAGAAGACCUCGCUGAUUGCUCGGUGCGCCGAGAC (SEQ ID NO: 561) |
| 62 | AUGGCAACAGACUCUCAUUGCGCGGUACGCCGCGAC (SEQ ID NO: 562) |
| 64 | GUCGCUCUCUAACGCUUGCCCAGUACGCUGGGAC (SEQ ID NO: 563) |
| 65 | GCTGGAAGACTCAATGATGGCTCCTTACGAGGAGAC (SEQ ID NO: 564) |
| 66, 67 | GGUUGAACCCUCAACAGAUUGCUCGGUAAGCCGAGAC (SEQ ID NO: 565) |
| 68 | CUUGAAAUCCUGUCAGAUUGCUCCCUUCGGGGAGAC (SEQ ID NO: 566) |
| 69, 70 | GCUGGAAGACUCAAUGAUGGCUCCUUACGAGGAGAC (SEQ ID NO: 567) |
| 71 | CUAGGAACGCACGCAGAUUGCUCGGUACGCCGAGAC (SEQ ID NO: 568) |
| 72 | AUUGCAACGCCUAAAGAUUGCUCGAUACGUCGAGAC (SEQ ID NO: 569) |
| 73 | GUUCGGCRAYCCUUUGAUUGCUCAGUACGCUGAGAC (SEQ ID NO: 570) |
| 74, 89 | GUUGAACCUAGAUCAGAUGGCUCAGUACGCUGAGAC (SEQ ID NO: 571) |
| 75 | CCCUCAACACGUCAGAAAUGCCCGGCACGCCGGGAC (SEQ ID NO: 572) |
| 76 | GUCGCAAGACUCGAAUAAUUGCCCCUCUAUGGGGAC (SEQ ID NO: 573) |
| 77 | GCUGGGGACCGAUCCUGAUUGCUCGCUGCGGCGAGAC (SEQ ID NO: 574) |
| 78 | CUCUCAAUGGAUAACGAUUGCUCUCUACGGAGAGAC (SEQ ID NO: 575) |
| 79 | GUUGAACCCUCAACAGAUUGCUCGGUAAGCCGAGAC (SEQ ID NO: 576) |
| 80, 88 | GTTGAACCCTCAACAGATTGCTCGGTAAGCCGAGAC (SEQ ID NO: 577) |
| 81 | GTCGCAAGACTCGAATAATTGCCCCTCTATGGGGAC (SEQ ID NO: 578) |
| 82, 90 | GTCGGAACGCTCAACGATTGCCCCTCACGAGGGGAC (SEQ ID NO: 579) |
| 83, 87 | GTTGAACCTAGATCAGATGGCTCAGTACGCTGAGAC (SEQ ID NO: 580) |
| 84 | CTCTCAATGGATAACGATTGCTCTCTACGGAGAGAC (SEQ ID NO: 581) |
| 85 | ACTGAAACCACCAACGATTGCGCTCCTCGGAGCGAC (SEQ ID NO: 582) |
| 86 | ACCAAAACGACTATTGATTGCCCAGTACGCTGGGAC (SEQ ID NO: 583) |
| 89 | ACUGAAACCACCAACGAUUGCGCUCCUCGGAGCGAC (SEQ ID NO: 584) |
| 91, 92 | GGTTGAACCCTCAACAGATTGCTCGGTAAGCCGAGAC (SEQ ID NO: 585) |
| 93, 96, 100 | GUUAGAAUAUAACCCUGUUUGUAGGGGUAAUAAAA (SEQ ID NO: 586) |
| 94, 103-104 | AGUUGUGUCUACCUCCUUUUUGAGAGGUAAAAACAAC (SEQ ID NO: 587) |
| 95 | AUUUGACUAUACCCCUAUUUGAGAGGGGAAUAAAA (SEQ ID NO: 588) |
| 97 | AUUGGAAUAUAACCCCGUUUGUAGGGGUAGUAAAA (SEQ ID NO: 589) |
| 98 | UUGACUACACUCUCUAUCUCUUAGGGAGACUGAAA (SEQ ID NO: 590) |
| 99, 101 | GUUUGAAUAUAACCCUGUUUGUAGGGGUAAUAAAA (SEQ ID NO: 591) |
| 102 | GAUAGAAUAUAACCCUGUUUGUAGGGGUAAUAAAA (SEQ ID NO: 592) |
| 103 | AGUCAGACUAUACCCUCGUUUGUAGGGGGAAUAAAAC (SEQ ID NO: 593) |
| 103-104 | GUUGCAUCUGCCUGCUGUUUGCAAGGUAAAAACAAC (SEQ ID NO: 594) |
| 103-104 | GAUUUAGACUACCCCAAAAACGAAGGGGACUAAAACC (SEQ ID NO: 595) |
| 103-104 | GUUUUAGUCCCCUUCGUUUUUGGGGUAGUCUAAAUC (SEQ ID NO: 596) |
| 103-104 | GUUGUAACUGCCCUUAUUUUGAAGGGUAAACACAAC (SEQ ID NO: 597) |
| 104 | GGUUAGAAUAUAACCCUGUUUGUAGGGGUAAUAAAAC (SEQ ID NO: 598) |
| 105 | AGAAGAAGGAUUGGGAC (SEQ ID NO: 599) |
| 106, 108-109 | AAAACAAGGAUUGAAAC (SEQ ID NO: 600) |
| 107 | CGUACGUGGAUUGAAAC (SEQ ID NO: 601) |
| 108 | GUCUGAAAGCUACUUCCAAUAAAACAAGGAUUGAAAC (SEQ ID NO: 602) |
| 110 | CUUGCAGGCCUUGAAUAGAGGAGUUAAGGAAUGCAAC (SEQ ID NO: 603) |
| 112 | GAAUGAAGGAAUGCAAC (SEQ ID NO: 604) |
| 119 | CUUUCAUACUCAGAACAAAGGGAUUAAGGAAUGCAAC (SEQ ID NO: 605) |
| 120 | AUUUCAUACUCAGAACAAAGGGAUUAAGGAAUGCAAC (SEQ ID NO: 606) |
| 126 | GUUGCGCGAAUAGAAUAAAGGAAUUAAGGAAUGCAAC (SEQ ID NO: 607) |
| 151 | GCUAGCCCGUGCGCGCAGGGACGAGUGG (SEQ ID NO: 608) |
| 240 | GAAUUUCUACUAUUGUAGAU (SEQ ID NO: 609) |
| 241-246 | AGAUUUCUACUUUUGUAGAU (SEQ ID NO: 610) |
| 405, 408, | AGGUACAUCCAAC (SEQ ID NO: 611) |
| 416, 436 | |
| 406, 412 | UGCGGUGUAAUUCGAGG (SEQ ID NO: 612) |
| 407 | GAUGUGAACGACCUUUUUUUGCGGUGUGCUUCGAGG (SEQ ID NO: 613) |
| 409 | GGUGGAUAUCAUCUUAAAAAGUGAGGUACAUCCAAC (SEQ ID NO: 614) |
| 412 | GGUGUGAACGACCUUUUUUUGCGGUGUAAUUCGAGG (SEQ ID NO: 615) |
| 412 | UUGCGGUGUACUUCGAGG (SEQ ID NO: 616) |
| 415 | GAUAUAUCUUGUAUGCAUAUGUAGGUUGUGAGAAUGUGAACGACCUUC |
| | UUUUGCGGUGUACUUCGAGG (SEQ ID NO: 617) |
| 417 | AAGGUUGAUACAGC (SEQ ID NO: 618) |
| 418 | GCUGUAAGUCAUGGAAAAAUGGUGAGUACAUCCAAC (SEQ ID NO: 619) |
| 418 | AUGGUGAGUACAUCCAAC (SEQ ID NO: 620) |
| 419 | GAGCACAUCCAAC (SEQ ID NO: 621) |
| 420, 428, 432 | GGGUACAUCCAAC (SEQ ID NO: 622) |
| 421 | GUUGCGUUUGCCCGUGAUUUCGGGUGUGUAUACAGG (SEQ ID NO: 623) |
| 423 | AGGUAUAUCCAAC (SEQ ID NO: 624) |
| 424 | GGCGUAUGUCUACCUGAAAAAGAAGGUAUAUCCAAC (SEQ ID NO: 625) |
| 426 | GGCUACAUACAGC (SEQ ID NO: 626) |
| 427 | GGUGUAUGUGCACCAUAUAUGUAGGUGACAUACAGC (SEQ ID NO: 627) |
| 430 | GAUGUGAACGACCUUUUUUUGCGGUGUACUUCGAGG (SEQ ID NO: 628) |
| 430 | GUGUACUUCGAGG (SEQ ID NO: 629) |
| 431 | GAUGUAAAUCAUCUAUAAAAGAAAGGUACAUCCAAC (SEQ ID NO: 630) |
| 431 | GGUACAUCCAAC (SEQ ID NO: 631) |
| 434 | GCUGCACUGCACCGCCCAUUGAUGGUGUGCUCUAGG (SEQ ID NO: 632) |
| 435 | AUUGUAGGCGACCUUUUUUUGCGAUGUAGUUCGAGG (SEQ ID NO: 633) |
| 435 | AUGUAGUUCGAGG (SEQ ID NO: 634) |
| 436 | AGUGUAUGAUUACCUGUAGUAUGAGGUACAUCCAAC (SEQ ID NO: 635) |
| 442 | GCUGCAAGAGCUCCUAAUUUGAGGGGUGCAUACAGG (SEQ ID NO: 636) |
| 443 | GAUAGUUUUAACUUCCAUUUGAAAUGUAAAUGCAAC (SEQ ID NO: 637) |
| 443 | AUGUAAAUGCAAC (SEQ ID NO: 638) |
| 77 | CCUGAUUGCUCGCUGCGGCGAGAC (SEQ ID NO: 507) |

In some embodiments, the repeat sequence comprises one or more nucleotide alterations at one or more positions in any one of the sequences of TABLE 3. Alternative nucleotides can be any one or more of A, C, G, T or U, or a deletion, or an insertion.

In some embodiments, viral vectors described herein comprise a nucleotide sequence that encodes an effector protein and/or a nucleotide sequence encoding a guide nucleic acid, wherein the effector protein binds to the guide nucleic acid. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences recited in TABLE 1. In some embodiments, the guide nucleic acid comprises a repeat sequence comprising a nucleotide sequence that has at least at least 65%, at least 70%, at least 80%, at least 90%, at least 92%, at least 95%, at least 97%, or at least 99%, or 100% sequence identity to any one of the sequences recited in recited in TABLE 3. In some embodiments, the effector protein binds to the guide nucleic acid comprising the corresponding repeat sequence as identified in TABLE 3. In some embodiments, the effector protein recognizes a corresponding PAM sequence as identified in TABLE 1.1. In some embodiments, the effector protein further comprises a NLS sequence having at least 65%, at least 70%, at least 75%, at least 80%, at least 85%, at least 90%, at least 95%, at least 97%, or at least 98%, at least 99%, or 100% sequence identity to any one of the sequences in TABLE 2.

### Spacer Sequence

In some embodiments, the compositions, systems and methods described herein, including an AAV vector, comprise a guide nucleic acid, or a nucleic acid encoding the guide nucleic acid, comprising a spacer sequence. The spacer sequence of the guide nucleic acid may comprise complementarity with (e.g., hybridize to) a target region of a target nucleic acid. In some embodiments, complementary or complementarity with reference to a nucleic acid molecule or nucleotide sequence, is the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

It is understood that the sequence of a spacer region need not be 100% complementary to that of a target sequence of a target nucleic acid to hybridize or hybridize specifically to the target sequence. The spacer sequence may comprise at least one nucleotide that is not complementary to the corresponding nucleotide of the target sequence. In some embodiments, the guide nucleic acid may comprise at least one uracil between nucleic acid residues 5 to 20 of the spacer region that is not complementary to the corresponding nucleotide of the target sequence. The guide nucleic acid may comprise at least one uracil between nucleic acid residues 5 to 9, 10 to 14, or 15 to 20 of the spacer region that is not complementary to the corresponding nucleotide of the target sequence.

In some cases, the spacer sequence is 15-28 linked nucleosides in length. In some cases, the spacer sequence is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleosides in length. In some cases, the spacer sequence is 18-24 linked nucleosides in length. In some cases, the spacer sequence is at least 15 linked nucleosides in length. In some cases, the spacer sequence is at least 16, 18, 20, or 22 linked nucleosides in length. In some cases, the spacer sequence comprises at least 10, 11, 12, 13, 14, 15, 16, 17, 18, 19, 20, 21, 22, 23, 24, 25, 26, 27, 28, 29, or 30 nucleotides. In some cases, the spacer sequence is at least 17 linked nucleosides in length. In some cases, the spacer sequence is at least 18 linked nucleosides in length. In some cases, the spacer sequence is at least 20 linked nucleosides in length. In some cases, the spacer sequence is at least 80%, at least 85%, at least 90%, at least 95% or 100% complementary to a target region of the target nucleic acid. In some cases, the spacer sequence is 100% complementary to the target region of the target nucleic acid. In some cases, the spacer sequence comprises at least 15 contiguous nucleobases that are complementary to the target nucleic acid. TABLE 4 shows exemplary spacer sequences.

TABLE 4
Exemplary Spacer Sequences

| SEQ | | SEQ | |
|---|---|---|---|
| ID | | ID | |
| NO: | Spacer Sequences | NO: | Spacer Sequences |
| 224 | UCUAGGCGCCCGCUAAGUUC | 264 | GAAGACACAGCUGGGGAGCU |
| 225 | CCCGGGUAAGCCUGUCUGCU | 265 | UCCUGGAGAGAACAGGCAAU |
| 226 | CGUGCUGUUUCCUCCCCACG | 266 | GGGACGAGGGUGUCUCGCAG |
| 227 | GUGCCUUAGUUUCUUCAUCU | 267 | UUGAGCUGCCUGAAUCUCCC |
| 228 | GGGGGCGGGGGGGAGAAAAA | 268 | CUCAAGGCAGCCCUGUGGAG |
| 229 | GCGCCCUCCGAUCUGGGGUG | 269 | GAGGUGUCCUCAUGUGGAGA |
| 247 | GUGAGGAAGCACCUGAGCCC | 270 | UCCCCGAUCUUGUUCUCACU |
| 248 | GGUUCUGGGAGGAAAAGUCC | 271 | GGAGGUCAGGGCAGGUUCAG |
| 249 | GUGAGAGUAUGGAGAUGCCA | 272 | CAGCUCUCAGCCACCUUCCC |
| 250 | AGCCUUUCAAAGCCAAGUCC | 273 | ACCAGCUUGACAUCACAGGA |
| 251 | AUGGUGAACUUCUCCUCUGC | 274 | AGAAAAGCUUUGAAACAGGU |
| 252 | GGACCUAAAGAAACUGGAGU | 275 | GAACCCAAUCACUGACAGGU |
| 253 | CUCGCUGCAUCCCUGCUCAG | 276 | UAUCUGUAAAACCAAGAGGC |
| 254 | UGUGCUUCCGGACAUGGUGU | 277 | GUGAAUAGGCAGACAGACUU |
| 255 | CUCCAUCAGCCACUGACCUG | 278 | CGCUACUCUCUCUUUCUGGC |
| 256 | GCGAGUUUGUAGGCACCCAG | 279 | GAUGGAUGAAACCCAGACAC |
| 257 | CCGAGGCCCUGCCUUCGCUC | 280 | AGACAUGUAAGCAGCAUCAU |
| 258 | CUGCAUCCCUGCUCAGGCUA | 281 | GGCCGAGAUGUCUCGCUCCG |
| 259 | CUGUUAAGAAGCUCCAGGUA | 282 | CUCCGUGGCCUUAGCUGUGC |
| 260 | CAGUUGAUGGUGUCUGUGUC | 283 | GCCCGAAUGCUGUCAGCUUC |
| 261 | CAGAUGCAGUUAUUGUACAA | 284 | AUCUAUGAAAAAGACAGUGG |
| 262 | GCUCCCACGUCGCAGAUGCA | 327 | AUCCAACCUCCGUGGCCUUA |
| | | | GCUGUGC |
| 263 | CUGCCAGCCUUCGGAGGUGU | 234 | GAGCAACGGCGGAAGGU |

In some embodiments, the spacer sequence comprises one or more nucleotide alterations at one or more positions in any one of the sequences of TABLE 4. Alternative nucleotides can be any one or more of A, C, G, T or U, or a deletion, or an insertion.

In some embodiments, viral vectors described herein comprise a sequence that encodes an effector protein and/or a nucleotide sequence encoding a guide nucleic acid, wherein the effector protein binds to the guide nucleic acid. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences recited in TABLE 1. In some embodiments, the guide nucleic acid comprises a spacer sequence comprising a nucleotide sequence that has at least at least 65%, at least 70%, at least 80%, at least 90%, at least 92%, at least 95%, at least 97%, or at least 99%, or 100% sequence identity to any one of the sequences recited in recited in TABLE 4. In some embodiments, the effector protein recognizes a corresponding PAM sequence as identified in TABLE 1.1. In some embodiments, the effector protein further comprises a NLS sequence having at least 65%, at least 70%, at least 75%, at least 80%, at least 85%, at least 90%, at least 95%, at least 97%, or at least 98%, at least 99%, or 100% sequence identity to any one of the sequences in TABLE 2.

crRNA

In some embodiments, the compositions, systems and methods described herein, including an AAV vector, comprise an guide nucleic acid, or a nucleic acid encoding the guide nucleic acid, that imparts activity or sequence selectivity to the effector protein. In general, the guide nucleic acid comprises a CRISPR RNA (crRNA) that includes a nucleotide sequence (e.g., spacer sequence) that is at least partially complementary to a target nucleic acid. In some instances, the guide nucleic acid comprises a crRNA, at least a portion of which interacts with the effector protein. Accordingly, in some embodiments, the repeat sequence of the crRNA may interact with an effector protein, allowing for the guide nucleic acid and the effector protein to form an RNP complex.

In general, crRNAs comprise a spacer sequence that hybridizes to a target region (e.g., target sequence) of a target nucleic acid, and a repeat sequence that interacts with the effector protein. In some embodiments, a crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpfl, from Type V systems). A crRNA may also be specifically generated outside of processing of a pre-crRNA and individually contacted to an effector protein in vivo or in vitro.

In some embodiments, a guide nucleic acid is a crRNA. In some embodiments, the crRNA comprises a repeat sequence and a spacer sequence. In some instances, the crRNA of the guide nucleic acid comprises a repeat sequence and a spacer sequence, wherein the repeat sequence is bound by the effector protein and the spacer sequence hybridizes to a target region of the target nucleic acid. In some embodiments, a repeat sequence of a crRNA interacts with the effector protein.

In some instances, the length of the crRNA is not greater than about 40, about 45, about 50, about 55, about 60, about 65, about 70 or about 75 linked nucleosides. In some instances, the length of the crRNA is about 30 to about 120 linked nucleosides. In some instances, the length of a crRNA is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleosides. In some instances, the length of a crRNA is about 40, about 45, about 50, about 55, about 60, about 65, about 70 or about 75 linked nucleosides. An exemplary crRNA includes a nucleotide sequence of GGAUUGCUCCUUACGAGGAGACGAGCAACGGCGGAAGGU (SEQ ID NO: 235).

In some embodiments, viral vectors described herein comprise a nucleotide sequence that encodes crRNA having at least at least 65%, at least 70%, at least 80%, at least 90%, at least 92%, at least 95%, at least 97%, or at least 99%, or 100% sequence identity to a nucleotide sequences recited in TABLE 3, TABLE 4, or combinations thereof. In some embodiments, viral vectors described herein comprise a nucleotide sequence that encodes crRNA comprising a repeat sequence and a spacer sequence. In some embodiments, the repeat sequence comprises a nucleotide sequence having at least at least 65%, at least 70%, at least 80%, at least 90%, at least 92%, at least 95%, at least 97%, or at least 99%, or 100% sequence identity to any one of the sequences recited in TABLE 3. In some embodiments, the spacer sequence comprises a nucleotide sequence having at least at least 65%, at least 70%, at least 80%, at least 90%, at least 92%, at least 95%, at least 97%, or at least 99%, or 100% sequence identity to any one ofthe sequences recited in TABLE 4.

In some embodiments, viral vectors described herein comprise a sequence that encodes an effector protein and/or a nucleotide sequence encoding a crRNA, wherein the effector protein binds to the crRNA. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences recited in TABLE 1. In some embodiments, the crRNA comprises a spacer sequence comprising a nucleotide sequence that has at least at least 65%, at least 70%, at least 80%, at least 90%, at least 92%, at least 95%, at least 97%, or at least 99%, or 100% sequence identity to any one of the sequences recited in TABLE 4. In some embodiments, the crRNA comprises a repeat sequence comprising a nucleotide sequence that has at least at least 65%, at least 70%, at least 80%, at least 90%, at least 92%, at least 95%, at least 97%, or at least 99%, or 100% sequence identity to any one of the sequences recited in TABLE 3. In some embodiments, the effector protein binds to the crRNA comprising the corresponding repeat sequence as identified in TABLE 3. In some embodiments, the effector protein recognizes a corresponding PAM sequence as identified in TABLE 1.1. In some embodiments, the effector protein further comprises a NLS sequence having at least at least 65%, at least 70%, at least 80%, at least 90%, at least 92%, at least 95%, at least 97%, or at least 99%, or 100% sequence identity to any one of the sequences recited in TABLE 2.

tracrRNA

In some embodiments, the compositions, systems and methods described herein, including an AAV vector, comprise a trans-activating crRNA (tracrRNA) or a nucleic acid encoding the tracrRNA, and a guide nucleic acid (e.g., in a dual nucleic acid system), and an effector protein, wherein at least a portion of tracrRNA interacts with the effector protein. A tracrRNA may include deoxyribonucleotides, ribonucleotides, or a combination thereof. In some embodiments, the tracrRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the tracrRNA comprises a pseudoknot (e.g., a secondary structure comprising a stem at least partially hybridized to a second stem or half-stem secondary structure). An effector protein may recognize a tracrRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, a tracrRNA may comprise a repeat hybridization region and a hairpin region. The repeat hybridization region may be positioned 3′ of the hairpin region. The hairpin region may comprise a first sequence, a second sequence that is reverse complementary to the first sequence, and a stem-loop linking the first sequence and the second sequence. A tracrRNA may also form a secondary structure (e.g., one or more hairpin loops) that facilitates the binding of an effector protein to a guide nucleic acid and/or modification activity of an effector protein on a target nucleic acid (e.g., a hairpin region).

In some instances, a length of a tracrRNA is not greater than 50, 56, 68, 71, 73, 95, or 105 linked nucleosides. In some instances, the length of a tracrRNA is about 30 to about 120 linked nucleosides. In some instances, the length of a tracrRNA is about 50 to about 105, about 50 to about 95, about 50 to about 73, about 50 to about 71, about 50 to about 68, or about 50 to about 56 linked nucleosides. In some instances, the length of a tracrRNA is 56 to 105 linked nucleosides, from 56 to 105 linked nucleosides, 68 to 105 linked nucleosides, 71 to 105 linked nucleosides, 73 to 105 linked nucleosides, or 95 to 105 linked nucleosides. In some instances, the length of a tracrRNA is 40 to 60 nucleotides. In some instances, the length of the tracrRNA is 50, 56, 68, 71, 73, 95, or 105 linked nucleosides. In some instances, the length of the tracrRNA is 50 nucleotides.

In some embodiments, the tracrRNA comprises a nucleotide sequence of UGGGGCAGUUGGUUGCCCUUAGCCUGAGGCAUUUAUUGCACUCGGGAAGUACCAUUUCU CA (SEQ ID NO: 231). In some embodiments, the tracrRNA comprises a nucleotide sequence of UGGGGCAGUUGGUUGCCCUUAGCCUGAGGCAUUUAUUGCACUCGGGAAUCA (SEQ ID NO: 368). In some embodiments, the tracrRNA comprises a nucleotide sequence having at least at least 65%, at least 70%, at least 80%, at least 90%, at least 92%, at least 95%, at least 97%, or at least 99%, or 100% sequence identity to any one of the sequences of SEQ ID NO: 231 or SEQ ID NO: 368.

In some embodiments, the tracrRNA may be separate from but forms a complex with a guide nucleic acid (e.g., crRNA) and an effector protein. In some embodiments, the repeat hybridization region may hybridize to all or part of the sequence of the repeat of the guide nucleic acid (e.g., crRNA). In some embodiments, the tracrRNA may hybridize to a portion of the guide nucleic acid that does not hybridize to the target nucleic acid. An exemplary tracrRNA may comprise, from 5′ to 3′, a 5′ region, a hairpin region, a repeat hybridization region, and a 3′ region. In some cases, the 5′ region may hybridize to the 3′ region. In some instances, a tracrRNA may comprise an unhybridized region at the 3′ end of the tracrRNA. The unhybridized region may have a length of about 1, about 2, about 3, about 4, about 5, about 6, about 7, about 8, about 9, about 10, about 12, about 14, about 16, about 18, or about 20 linked nucleosides. In some instances, the length of the un-hybridized region is 0 to 20 linked nucleosides.

In some embodiments, an guide nucleic acid is linked to a tracrRNA, at least a portion of which interacts with the effector protein. A guide nucleic acid may comprise a repeat sequence or at least a portion thereof, wherein the repeat sequence or at least a portion thereof may be coupled to a tracrRNA. The tracrRNA may comprise deoxyribonucleosides or chemically modified nucleosides in addition to ribonucleosides. In some embodiments, the guide nucleic acid is a crRNA. In some embodiments, the tracrRNA may be (covalently) linked to the crRNA. In some instances, the crRNA and tracrRNA are linked by one or more linked nucleotides. In some embodiments, the crRNA comprises a repeat sequence and a spacer sequence. In some embodiments, the tracrRNA and the repeat sequence of crRNA are linked by one or more nucleotides. Accordingly, in some embodiments, the tracrRNA may be attached (e.g., covalently) by one or more nucleotides to the guide nucleic acid. Accordingly, in some instances, the 5′ region does not hybridize to the 3′ region. In some cases, the 3′ region is covalently linked to the crRNA (e.g., through a phosphodiester bond). In some embodiments, the crRNA and tracrRNA are linked by a phosphodiester bond. Accordingly, in some embodiments, a viral vector comprises a guide nucleic acid or a sequence encoding the guide nucleic acid comprising a tracrRNA or a sequence encoding a tracrRNA.

In some instances, the guide nucleic acid does not comprise a tracrRNA. In some embodiments, the guide nucleic acid is a crRNA. In some instances, the crRNA and tracrRNA are not covalently linked. In some embodiments, the crRNA and tracrRNA sequences are provided as a separate molecule. In some instances, a crRNA and tracrRNA function as two separate, unlinked molecules. In some embodiments, a portion of tracrRNA interacts with an effector protein, wherein the portion does not hybridize to crRNA. In some embodiments, a viral vector comprises a guide nucleic acid, such as a crRNA, wherein the guide nucleic acid does not comprise a tracrRNA. In some instances, the crRNA and the tracrRNA are separate polynucleotides. Accordingly, in some embodiments, a viral vector comprises a tracrRNA or a sequence encoding a tracrRNA. In some embodiments, a viral vector comprises a crRNA or a sequence encoding a crRNA.

In some cases, an effector protein does not require a tracrRNA to locate and/or cleave a target nucleic acid.

sgRNA

In some embodiments, the compositions, systems and methods described herein, including an AAV vector, comprise a single guide nucleic acid, also referred to as a single guide RNA (sgRNA), comprising a handle sequence and a spacer sequence. In some embodiments, a handle sequence and a spacer sequence are (covalently) linked to form a sgRNA. A sgRNA may include deoxyribonucleosides, ribonucleosides, chemically modified nucleosides, or any combination thereof. A sgRNA may also include a nucleotide sequence that forms a secondary structure (e.g., one or more hairpin loops) that facilitates the binding of an effector protein to the sgRNA and/or modification activity of an effector protein on a target nucleic acid (e.g., a hairpin region). Such a sequence can be contained within a handle sequence as described herein. A sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, a sgRNA comprises a linker sequence. In some embodiments, the sgRNA comprises a handle sequence and a spacer sequence, wherein the handle sequence comprises the linker sequence. TABLE 5 shows exemplary linker sequence.

TABLE 5
Exemplary Linker Sequence

| SEQ ID NO: | Linker Sequence |
|---|---|
| 230 | GAAA |

In some embodiments, a portion of sgRNA that is referred to as a handle sequence interacts with the effector protein. In some instances, the portion of a single guide nucleic acid comprising a handle sequence comprises one or more of at least a portion of or all of a tracrRNA sequence and at least a portion of or all of a repeat sequence. The tracrRNA may be attached (e.g., covalently) by an artificial linker to the repeat sequence within a single guide nucleic acid. Accordingly, in some instances, the handle sequence further comprises a linker sequence, wherein the linker sequence comprises one or more nucleotides. Alternatively, in some instances, a region of sgRNA comprising a linker, at least a portion of or all of a tracrRNA, and at least a portion of or all of a repeat sequence, the region is referred to herein as the handle sequence.

In some embodiments, the handle sequence of a sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize a sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

In some embodiments, the length of a handle sequence in a sgRNA is not greater than 50, 56, 66, 67, 68, 69, 70, 71, 72, 73, 95, or 105 linked nucleotides. In some embodiments, the length of a handle sequence in a sgRNA is about 30 to about 120 linked nucleotides. In some embodiments, the length of a handle sequence in a sgRNA is about 50 to about 105, about 50 to about 95, about 50 to about 73, about 50 to about 71, about 50 to about 70, or about 50 to about 69 linked nucleotides. In some embodiments, the length of a handle sequence in a sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in a sgRNA is 40 to 70 nucleotides. In some embodiments, the length of a handle sequence in a sgRNA is 50, 56, 58, 62, 66, 67, 68, 69, 70, 71, 72, 73, 75, 95, or 105 linked nucleotides. In some embodiments, the length of a handle sequence in a sgRNA is 69 nucleotides. In some embodiments, the length of a handle sequence in a sgRNA is up to 75 nucleotides.

TABLE 6 shows exemplary handle sequences. An exemplary handle sequence in a sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

TABLE 6
Exemplary Handle Sequences

| Effector | |
|---|---|
| Protein | |
| SEQ ID NO: | Handle Sequences |
| 23 | UGGUACAUCCAAC |
| | (SEQ ID NO: 215) |
| 23 | UGGGGCAGUUGGUUGCCCUUAGCCUGAGGCAUUUAUUGC |
| | ACUCGGGAAGUACCAUUUCUCA |
| | (SEQ ID NO: 231) |
| 23 | UGGGGCAGUUGGUUGCCCUUAGCCUGAGGCAUUUAUUGC |
| | ACUCGGGAAGUACCAUUUCUCAGAAA |
| | (SEQ ID NO: 232) |
| 23 | UGGGGCAGUUGGUUGCCCUUAGCCUGAGGCAUUUAUUGC |
| | ACUCGGGAAGUACCAUUUCUCAGAAAUGGUACAUCCAAC |
| | (SEQ ID NO: 214) |
| 23 | UGGGGCAGUUGGUUGCCCUUAGCCUGAGGCAUUUAUUGC |
| | ACUCGGGAAUCA |
| | (SEQ ID NO: 368) |

In some embodiments, the handle sequence comprises one or more nucleotide alterations at one or more positions in any one of the sequences of TABLE 6. Alternative nucleotides can be any one or more of A, C, G, T or U, or a deletion, or an insertion.

In some embodiments, viral vectors described herein comprise a nucleotide sequence that encodes a sgRNA comprising a handle sequence. In some embodiments, the handle sequence comprises a nucleotide sequence having at least at least 65%, at least 70%, at least 80%, at least 90%, at least 92%, at least 95%, at least 97%, or at least 99%, or 100% sequence identity to any one of the sequences recited in TABLE 3 and TABLE 6.

In some embodiments, viral vectors described herein comprise a sequence that encodes an effector protein, wherein the amino acid sequence of the effector protein is at least 65%, at least 70%, at least 75%, at least 80%, at least 85%, at least 90%, at least 95%, at least 97%, or at least 98%, at least 99%, or 100% identical to any one ofthe sequences recited in TABLE 1 binds to a sgRNA comprising a handle sequence and a spacer sequence. In some embodiments, the spacer sequence comprises a nucleotide sequence having at least at least 65%, at least 70%, at least 80%, at least 90%, at least 92%, at least 95%, at least 97%, or at least 99%, or 100% sequence identity to any one of the sequences recited in TABLE 4. In some embodiments, the handle sequence comprises a nucleotide sequence having at least at least 65%, at least 70%, at least 80%, at least 90%, at least 92%, at least 95%, at least 97%, or at least 99%, or 100% sequence identity to any one of the sequences recited in TABLE 3 and TABLE 4. In some embodiments, a repeat sequence of the handle sequence corresponds to the effector protein as identified in TABLE 3. In some embodiments, the effector protein recognizes a corresponding PAM sequence as identified in TABLE 1.1. In some embodiments, the effector protein comprises a NLS sequence having at least at least 65%, at least 70%, at least 80%, at least 90%, at least 92%, at least 95%, at least 97%, or at least 99%, or 100% sequence identity to any one of the sequences recited in TABLE 2.

In some embodiments, a viral vector comprises a sgRNA or a sequence encoding a sgRNA. In some embodiments, a viral vector comprises a sgRNA or a sequence encoding a sgRNA, wherein sgRNA comprises a handle sequence and a spacer sequence. In some embodiments, a sgRNA comprises a linker sequence. Accordingly, in some embodiments, the sgRNA comprises a handle sequence and a spacer sequence, wherein the handle sequence comprises the linker sequence. In some embodiments, a viral vector comprises a sgRNA or a sequence encoding a sgRNA. In some embodiments, a viral vector comprises a sgRNA or a sequence encoding a sgRNA, wherein sgRNA comprises a handle sequence, a spacer sequence and optionally a linker sequence as a part of the handle sequence.

TABLE 7 shows exemplary sgRNAs, wherein 1) different portions ofthe sgRNA handle sequence are shown, wherein a) capital letters indicate the tracrRNA derived region of the guide RNA, b) italicized letters indicate a linker, c) bold letters indicate the repeat sequence; and 2) the lowercase letters represent the spacer sequence.

TABLE 7
Exemplary guide sgRNAs

| SEQ ID NO: | sgRNA Sequences |
|---|---|
| 202 | UGGGGCAGUUGGUUGCCCUUAGCCUGAGGCAUUUAUUGCACUCGGGAAGUA |
| | CCAUUUCUCAGAAAUGGUACAUCCAACucuaggegcccgcuaaguuc |
| 203 | UGGGGCAGUUGGUUGCCCUUAGCCUGAGGCAUUUAUUGCACUCGGGAAGUA |
| | CCAUUUCUCAGAAAUGGUACAUCCAACcccggguaagccugucugcu |
| 204 | UGGGGCAGUUGGUUGCCCUUAGCCUGAGGCAUUUAUUGCACUCGGGAAGUA |
| | CCAUUUCUCAGAAAUGGUACAUCCAACcgugcuguuuccuccccacg |
| 205 | UGGGGCAGUUGGUUGCCCUUAGCCUGAGGCAUUUAUUGCACUCGGGAAGUA |
| | CCAUUUCUCAGAAAUGGUACAUCCAACgugccuuaguuucuucaucu |
| 206 | UGGGGCAGUUGGUUGCCCUUAGCCUGAGGCAUUUAUUGCACUCGGGAAGUA |
| | CCAUUUCUCAGAAAUGGUACAUCCAACggggggggggggagaaaaa |
| 207 | UGGGGCAGUUGGUUGCCCUUAGCCUGAGGCAUUUAUUGCACUCGGGAAGUA |
| | CCAUUUCUCAGAAAUGGUACAUCCAACgcgcccuccgaucuggggug |
| 329 | UGGGGCAGUUGGUUGCCCUUAGCCUGAGGCAUUUAUUGCACUCGGGAAGUA |
| | CCAUUUCUCAGAAAUGGUACAUCCAACgugaggaagcaccugagccc |
| 330 | UGGGGCAGUUGGUUGCCCUUAGCCUGAGGCAUUUAUUGCACUCGGGAAGUA |
| | CCAUUUCUCAGAAAUGGUACAUCCAACgguucugggaggaaaagucc |
| 331 | UGGGGCAGUUGGUUGCCCUUAGCCUGAGGCAUUUAUUGCACUCGGGAAGUA |
| | CCAUUUCUCAGAAAUGGUACAUCCAACgugagaguauggagaugcca |
| 332 | UGGGGCAGUUGGUUGCCCUUAGCCUGAGGCAUUUAUUGCACUCGGGAAGUA |
| | CCAUUUCUCAGAAAUGGUACAUCCAACagccuuucaaagccaagucc |
| 333 | UGGGGCAGUUGGUUGCCCUUAGCCUGAGGCAUUUAUUGCACUCGGGAAGUA |
| | CCAUUUCUCAGAAAUGGUACAUCCAACauggugaacuucuccucugc |
| 334 | UGGGGCAGUUGGUUGCCCUUAGCCUGAGGCAUUUAUUGCACUCGGGAAGUA |
| | CCAUUUCUCAGAAAUGGUACAUCCAACggaccuaaagaaacuggagu |
| 335 | UGGGGCAGUUGGUUGCCCUUAGCCUGAGGCAUUUAUUGCACUCGGGAAGUA |
| | CCAUUUCUCAGAAAUGGUACAUCCAACcucgcugcaucccugcucag |
| 336 | UGGGGCAGUUGGUUGCCCUUAGCCUGAGGCAUUUAUUGCACUCGGGAAGUA |
| | CCAUUUCUCAGAAAUGGUACAUCCAACugugcuuccggacauggugu |
| 337 | UGGGGCAGUUGGUUGCCCUUAGCCUGAGGCAUUUAUUGCACUCGGGAAGUA |
| | CCAUUUCUCAGAAAUGGUACAUCCAACcuccaucagccacugaccug |
| 338 | UGGGGCAGUUGGUUGCCCUUAGCCUGAGGCAUUUAUUGCACUCGGGAAGUA |
| | CCAUUUCUCAGAAAUGGUACAUCCAACgcgaguuuguaggcacccag |
| 339 | UGGGGCAGUUGGUUGCCCUUAGCCUGAGGCAUUUAUUGCACUCGGGAAGUA |
| | CCAUUUCUCAGAAAUGGUACAUCCAACccgaggcccugccuucgcuc |
| 340 | UGGGGCAGUUGGUUGCCCUUAGCCUGAGGCAUUUAUUGCACUCGGGAAGUA |
| | CCAUUUCUCAGAAAUGGUACAUCCAACcugcaucccugcucaggcua |
| 341 | UGGGGCAGUUGGUUGCCCUUAGCCUGAGGCAUUUAUUGCACUCGGGAAGUA |
| | CCAUUUCUCAGAAAUGGUACAUCCAACcuguuaagaagcuccaggua |
| 342 | UGGGGCAGUUGGUUGCCCUUAGCCUGAGGCAUUUAUUGCACUCGGGAAGUA |
| | CCAUUUCUCAGAAAUGGUACAUCCAACcaguugauggugucuguguc |
| 343 | UGGGGCAGUUGGUUGCCCUUAGCCUGAGGCAUUUAUUGCACUCGGGAAGUA |
| | CCAUUUCUCAGAAAUGGUACAUCCAACcagaugcaguuauuguacaa |
| 344 | UGGGGCAGUUGGUUGCCCUUAGCCUGAGGCAUUUAUUGCACUCGGGAAGUA |
| | CCAUUUCUCAGAAAUGGUACAUCCAACgcucccacgucgcagaugca |
| 345 | UGGGGCAGUUGGUUGCCCUUAGCCUGAGGCAUUUAUUGCACUCGGGAAGUA |
| | CCAUUUCUCAGAAAUGGUACAUCCAACcugccagccuucggaggugu |
| 346 | UGGGGCAGUUGGUUGCCCUUAGCCUGAGGCAUUUAUUGCACUCGGGAAGUA |
| | CCAUUUCUCAGAAAUGGUACAUCCAACgaagacacagcuggggagcu |
| 347 | UGGGGCAGUUGGUUGCCCUUAGCCUGAGGCAUUUAUUGCACUCGGGAAGUA |
| | CCAUUUCUCAGAAAUGGUACAUCCAACuccuggagagaacaggcaau |
| 348 | UGGGGCAGUUGGUUGCCCUUAGCCUGAGGCAUUUAUUGCACUCGGGAAGUA |
| | CCAUUUCUCAGAAAUGGUACAUCCAACgggacgagggugucucgcag |
| 349 | UGGGGCAGUUGGUUGCCCUUAGCCUGAGGCAUUUAUUGCACUCGGGAAGUA |
| | CCAUUUCUCAGAAAUGGUACAUCCAACuugagcugccugaaucuccc |
| 350 | UGGGGCAGUUGGUUGCCCUUAGCCUGAGGCAUUUAUUGCACUCGGGAAGUA |
| | CCAUUUCUCAGAAAUGGUACAUCCAACcucaaggcagcccuguggag |
| 351 | UGGGGCAGUUGGUUGCCCUUAGCCUGAGGCAUUUAUUGCACUCGGGAAGUA |
| | CCAUUUCUCAGAAAUGGUACAUCCAACgagguguccucauguggaga |
| 352 | UGGGGCAGUUGGUUGCCCUUAGCCUGAGGCAUUUAUUGCACUCGGGAAGUA |
| | CCAUUUCUCAGAAAUGGUACAUCCAACuccccgaucuuguucucacu |
| 353 | UGGGGCAGUUGGUUGCCCUUAGCCUGAGGCAUUUAUUGCACUCGGGAAGUA |
| | CCAUUUCUCAGAAAUGGUACAUCCAACggaggucagggcagguucag |
| 354 | UGGGGCAGUUGGUUGCCCUUAGCCUGAGGCAUUUAUUGCACUCGGGAAGUA |
| | CCAUUUCUCAGAAAUGGUACAUCCAACcagcucucagccaccuuccc |
| 355 | UGGGGCAGUUGGUUGCCCUUAGCCUGAGGCAUUUAUUGCACUCGGGAAGUA |
| | CCAUUUCUCAGAAAUGGUACAUCCAACaccagcuugacaucacagga |
| 356 | UGGGGCAGUUGGUUGCCCUUAGCCUGAGGCAUUUAUUGCACUCGGGAAGUA |
| | CCAUUUCUCAGAAAUGGUACAUCCAACagaaaagcuuugaaacaggu |
| 357 | UGGGGCAGUUGGUUGCCCUUAGCCUGAGGCAUUUAUUGCACUCGGGAAGUA |
| | CCAUUUCUCAGAAAUGGUACAUCCAACgaacccaaucacugacaggu |
| 358 | UGGGGCAGUUGGUUGCCCUUAGCCUGAGGCAUUUAUUGCACUCGGGAAGUA |
| | CCAUUUCUCAGAAAUGGUACAUCCAACuaucuguaaaaccaagaggc |
| 359 | UGGGGCAGUUGGUUGCCCUUAGCCUGAGGCAUUUAUUGCACUCGGGAAGUA |
| | CCAUUUCUCAGAAAUGGUACAUCCAACgugaauaggcagacagacuu |
| 360 | UGGGGCAGUUGGUUGCCCUUAGCCUGAGGCAUUUAUUGCACUCGGGAAGUA |
| | CCAUUUCUCAGAAAUGGUACAUCCAAguacucucucuuucuggc |
| 361 | UGGGGCAGUUGGUUGCCCUUAGCCUGAGGCAUUUAUUGCACUCGGGAAGUA |
| | CCAUUUCUCAGAAAUGGUACAUCCAACgauggaugaaacccagacac |
| 362 | UGGGGCAGUUGGUUGCCCUUAGCCUGAGGCAUUUAUUGCACUCGGGAAGUA |
| | CCAUUUCUCAGAAAUGGUACAUCCAACagacauguaagcagcaucau |
| 363 | UGGGGCAGUUGGUUGCCCUUAGCCUGAGGCAUUUAUUGCACUCGGGAAGUA |
| | CCAUUUCUCAGAAAUGGUACAUCCAACggccgagaugucucgcuccg |
| 364 | UGGGGCAGUUGGUUGCCCUUAGCCUGAGGCAUUUAUUGCACUCGGGAAGUA |
| | CCAUUUCUCAGAAAUGGUACAUCCAACcuccguggccuuagcugugc |
| 365 | UGGGGCAGUUGGUUGCCCUUAGCCUGAGGCAUUUAUUGCACUCGGGAAGUA |
| | CCAUUUCUCAGAAAUGGUACAUCCAACgcccgaaugcugucagcuuc |
| 366 | UGGGGCAGUUGGUUGCCCUUAGCCUGAGGCAUUUAUUGCACUCGGGAAGUA |
| | CCAUUUCUCAGAAAUGGUACAUCCAACaucuaugaaaaagacagugg |
| 367 | UGGGGCAGUUGGUUGCCCUUAGCCUGAGGCAUUUAUUGCACUCGGGAAUCA |
| | AUCCAACCUCCGUGGCCUUAGCUGUGC |

In some embodiments, the sgRNA comprises one or more nucleotide alterations at one or more positions in any one of the sequences of TABLE 7. Alternative nucleotides can be any one or more of A, C, G, T or U, or a deletion, or an insertion.

In some embodiments, the sgRNA sequence comprises at least 65%, at least 70%, at least 80%, at least 90%, at least 92%, at least 95%, at least 97%, or at least 99%, or 100% sequence identity to any one of sequence recited in TABLE 3, TABLE 4, TABLE 6 and TABLE 7.

In some embodiments, viral vectors described herein comprise a nucleotide sequence that encodes sgRNA having at least at least 65%, at least 70%, at least 80%, at least 90%, at least 92%, at least 95%, at least 97%, or at least 99%, or 100% sequence identity to any one of the nucleotide sequences recited in TABLE 3, TABLE 4, TABLE 6 and TABLE 7. In some embodiments, viral vectors described herein comprise a nucleotide sequence that encodes sgRNA comprising a handle sequence and a spacer sequence. In some embodiments, the handle sequence comprises a nucleotide sequence having at least at least 65%, at least 70%, at least 80%, at least 90%, at least 92%, at least 95%, at least 97%, or at least 99%, or 100% sequence identity to any one of the sequences recited in TABLE 3 and TABLE 6. In some embodiments, a repeat sequence of the handle sequence comprises a nucleotide sequence having at least at least 65%, at least 70%, at least 80%, at least 90%, at least 92%, at least 95%, at least 97%, or at least 99%, or 100% sequence identity to any one of the sequences recited in TABLE 3. In some embodiments, the spacer sequence comprises a nucleotide sequence having at least at least 65%, at least 70%, at least 80%, at least 90%, at least 92%, at least 95%, at least 97%, or at least 99%, or 100% sequence identity to any one of the sequences recited in TABLE 4.

In some embodiments, viral vectors described herein comprise a sequence that encodes an effector protein and/or a nucleotide sequence encoding a sgRNA, wherein the effector protein binds to the sgRNA. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences recited in TABLE 1. In some embodiments, the sgRNA comprises a spacer sequence comprising a nucleotide sequence that has at least at least 65%, at least 70%, at least 80%, at least 90%, at least 92%, at least 95%, at least 97%, or at least 99%, or 100% sequence identity to any one of the sequences recited in TABLE 4. In some embodiments, the sgRNA comprises a handle sequence comprising a nucleotide sequence that has at least at least 65%, at least 70%, at least 80%, at least 90%, at least 92%, at least 95%, at least 97%, or at least 99%, or 100% sequence identity to any one of the sequences recited in TABLE 3 and TABLE 4. In some embodiments, the handle sequence comprises a repeat sequence comprising a nucleotide sequence that has at least at least 65%, at least 70%, at least 80%, at least 90%, at least 92%, at least 95%, at least 97%, or at least 99%, or 100% sequence identity to any one of the sequences recited in TABLE 3. In some embodiments, the effector protein binds to the sgRNA comprising the corresponding repeat sequence as identified in TABLE 3. In some embodiments, the effector protein recognizes a corresponding PAM sequence as identified in TABLE 1.1. In some embodiments, the effector protein further comprises a NLS sequence having at least at least 65%, at least 70%, at least 80%, at least 90%, at least 92%, at least 95%, at least 97%, or at least 99%, or 100% sequence identity to any one of the sequences recited in TABLE 2.

### VI. Donor Nucleic Acids

Compositions, systems, vectors, and methods described herein often comprise a donor nucleic acid or a use thereof. Donor nucleic acids may comprise a cDNA. The cDNA may encode a wildtype protein. The cDNA may encode a mutant protein. In some instances, the donor nucleic acid does not comprise a cDNA. In some instances, the donor nucleic acid comprises a regulatory element. Non-limiting examples of regulatory elements are a promoter and a transcription factor binding site. In some instances, the donor nucleic acid comprises or encodes a nucleic acid selected from a mRNA, an miRNA, an siRNA, an antisense oligonucleotide, a primer binding site, a protein tag, a detectable marker (e.g., a fluorescence marker), and a combination thereof.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

In some instances, the viral vector comprises a donor nucleic acid. The donor nucleic acid may be a homology directed repair template. In some instances, the donor nucleic acid comprises a gene or a portion thereof, wherein the sequence of the gene or portion thereof is a wildtype sequence. A wildtype sequence is a sequence that provides for a wildtype phenotype. In some embodiments, the donor nucleic acid is (designed or intended to be) incorporated into a target nucleic acid or target sequence.

In reference to a viral vector, a donor nucleic acid is a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome. As another example, when used in reference to the activity of an effector protein, the term donor nucleic acid refers to a sequence of nucleotides that will be or has been inserted at the site of cleavage by the effector protein (cleaving (hydrolysis of a phosphodiester bond) of a nucleic acid resulting in a nick or double strand break-nuclease activity). As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

In some embodiments, the viral vector comprises a donor nucleic acid. In some embodiments, the viral vector comprising the donor nucleic acid further comprises a nucleic acid encoding an effector protein having an amino acid sequence that is at least 65%, at least 70%, at least 75%, at least 80%, at least 85%, at least 90%, at least 95%, or 100% identical to any one of the sequences of TABLE 1. In some embodiments, the viral vector comprising the donor nucleic acid further comprises a nucleic acid encoding a guide nucleic acid having a sequence that is at least 65%, at least 70%, at least 75%, at least 80%, at least 85%, at least 90%, at least 95%, or 100% identical to any one of the sequences of TABLE 3, TABLE 4, TABLE 6, and TABLE 7. In some embodiments, the viral vector comprising the donor nucleic acid does not comprise one or more of a nucleic acid encoding the effector protein and the guide nucleic acid.

Donor nucleic acids of any suitable size may be integrated into a target nucleic acid or genome. In some instances, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some instances, donor nucleic acids are more than 500 kilobases (kb) in length.

In some instances, the length of the donor nucleic acid is about 100 to about 200, about 200 to about 300, about 300 to about 400, about 400 to about 500, about 500 to about 600, about 600 to about 700, about 700 to about 800, about 800 to about 900, about 900 to about 1000, about 1000 to about 1100, about 1100 to about 1200, about 1200 to about 1300, about 1300 to about 1400, about 1400 to about 1500, about 1500 to about 1600, about 1600 to about 1700, about 1700 to about 1800, about 1800 to about 1900, or about 1900 to about 2000 linked nucleosides. In some instances, the length of the donor nucleic acid is about 300 to about 600 linked nucleosides. In some instances, the length of the donor nucleic acid is about 1 kb to about 2 kb, about 1 kb to about 1.5 kb, or about 1.5 kb to about 2 kb. In some instances, the length of the donor nucleic acid is about 1 kb to about 1.2 kb, about 1.2 kb to about 1.6 kb, about 1 kb to about 1.2 kb, about 1.2 kb to about 1.4 kb, about 1.4 kb to about 1.6 kb, about 1.6 kb to about 1.8 kb, about 1.8 kb to about 2 kb. In some instances, the length of the donor nucleic acid is less than about 100 linked nucleosides. In some instances, the length of the donor nucleic acid is at least about 10 linked nucleosides.

In some instances, the donor nucleic acid is at least 75%, at least 80%, at least 85%, at least 90%, at least 95%, at least 98%, at least 99%, or 100% identical to a gene or an equal length portion thereof. In some instances, the gene encodes a cytokine. In some instances, the gene encodes a DNA damage repair gene. In some instances, the gene encodes a DNA mismatch repair gene. Non-limiting examples of genes are ABCA4, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, ANGPTL3, AQP2, ARG1, ARSA, ARSB, ASL, ASNS, ASPA, ASS1, ATM, ATP6V1B1, ATP7A, ATP7B, ATRX, ATTR, ATXN1, B2M, BBS1, BBS10, BBS12, BBS2, BCL11A, BCKDHA, BCKDHB, BCS1L, BLM, BSND, CAPN3, CBS, CCR5, CDH23, CEP290, CERKL, CFTR, CHM, CHRNE, CIITA, CLN1, CLN3, CLN5, CLN6, CLN8, CLRN1, CNGB3, COL27A1, COL4A3, COL4A4, COL4A5, COL7A1, CPS1, CPT1A, CPT2, CRB1, CRX, CTLA-4, CTNS, CTSK, CXCR4, CYBA, CYBB, CYP11B1, CYP11B2, CYP17A1, CYP19A1, CYP27A1, DBT, DCLRE1C, DHCR7, DHDDS, DLD, DM1, DMD, DNAH5, DNAI1, DNAI2, DYSF, EDA, EIF2B5, ELOVL4, EMD, ERCC6, ERCC8, ESCO2, ETFA, ETFDH, ETHE1, EVC, EVC2, EYS, F8, F9, FAH, FAM161A, FANCA, FANCC, FANCG, FH, FKRP, FKTN, FMR1, FUS, FXN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, GUCY2D, ADHA, HAX1, HBA1, HBA2, HBB, HBG, HD, HEXA, HEXB, HGSNAT, HLA, HLCS, HMGCL, HOGA1, HPD, HPS1, HPS3, HSD17B4, HSD3B2, HTT, HYAL1, HYLS1, IDS, IDUA, IKBKAP, IL7R, IL2RG, IVD, KCNJ11, KLKB1, LAG3, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PCSK9, PD1, PDHA1, PDHB, PEX1, PEX10, PEX12, PEX2, PEX6, PEX7, PFKM, PHGDH, PIGA, PKHD1, PMM2, POMGNT1, PPT1, PROP1, PRPS1, PSAP, PTS, PUS1, PYGM, RAB23, RAG1, RAG2, RAPSN, RARS2, RDH12, RHO, RMRP, RPE65, RPGR, RIP1L, RPS19, RS1, RTEL1, SACS, SAMHD1, SBDA, SEPSECS, SERPINA1, SCNA, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMN1, SMPD1, STAR, SUMF1, TAT, TCIRG1, TECPR2, TFR2, TGFBR2, TGFBi, TGM1, TH, TIGIT, TIM3, TMEM216, TPP1, TRAC, TRMU, TSFM, TTPA, TYMP, USH1B, USH1C, USH2A, VLGR, VPS13A, VPS13B, VPS45, VRK1, VSX2, WAS, WNT10A, XPA, XPC, and ZFYVE26.

### VII. Target Nucleic Acids

In some embodiments, a target nucleic acid is a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The guide nucleic acid may bind to a target nucleic acid (e.g., a single strand of a target nucleic acid) or a portion thereof. The guide nucleic acid may bind to a target nucleic acid such as a nucleic acid from a bacterium, a virus, a parasite, a protozoon, a fungus or other agents responsible for a disease, or an amplicon thereof. The target nucleic acid may comprise a mutation, such as a single nucleotide polymorphism (SNP). A mutation may confer for example, resistance to a treatment, such as antibiotic treatment. The guide nucleic acid may bind to a target nucleic acid, such as DNA or RNA, from a cancer gene or gene associated with a genetic disorder, or an amplicon thereof, as described herein.

In some embodiments, the guide nucleic acid can bind to a target sequence, wherein the target sequence is eukaryotic. The guide nucleic acid may bind to a target nucleic acid, such as DNA or RNA, from a cancer gene or gene associated with a genetic disorder, or an amplicon thereof, as described herein. In some embodiments, the guide nucleic acid comprises a region that is complementary to an equal length portion of a target nucleic acid.

In some instances, the guide nucleic acid comprises a region that is complementary to an equal length portion of a gene selected from: ABCA4, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, ANGPTL3, AQP2, ARG1, ARSA, ARSB, ASL, ASNS, ASPA, ASS1, ATM, ATP6V1B1, ATP7A, ATP7B, ATRX, ATTR, ATXN1, B2M, BBS1, BBS10, BBS12, BBS2, BCL11A, BCKDHA, BCKDHB, BCS1L, BLM, BSND, CAPN3, CBS, CCR5, CDH23, CEP290, CERKL, CFTR, CHM, CHRNE, CIITA, CLN1, CLN3, CLN5, CLN6, CLN8, CLRN1, CNGB3, COL27A1, COL4A3, COL4A4, COL4A5, COL7A1, CPS1, CPT1A, CPT2, CRB1, CRX, CTLA-4, CTNS, CTSK, CXCR4, CYBA, CYBB, CYP11B1, CYP11B2, CYP17A1, CYP19A1, CYP27A1, DBT, DCLRE1C, DHCR7, DHDDS, DLD, DM1, DMD, DNAH5, DNAI1, DNAI2, DYSF, EDA, EIF2B5, ELOVL4, EMD, ERCC6, ERCC8, ESCO2, ETFA, ETFDH, ETHE1, EVC, EVC2, EYS, F8, F9, FAH, FAM161A, FANCA, FANCC, FANCG, FH, FKRP, FKTN, FMR1, FUS, FXN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, GUCY2D, ADHA, HAX1, HBA1, HBA2, HBB, HBG, HD, HEXA, HEXB, HGSNAT, HLA, HLCS, HMGCL, HOGA1, HPD, HPS1, HPS3, HSD17B4, HSD3B2, HTT, HYAL1, HYLS1, IDS, IDUA, IKBKAP, IL7R, IL2RG, IVD, KCNJ11, KLKB1, LAG3, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PCSK9, PD1, PDHA1, PDHB, PEX1, PEX10, PEX12, PEX2, PEX6, PEX7, PFKM, PHGDH, PIGA, PKHD1, PMM2, POMGNT1, PPT1, PROP1, PRPS1, PSAP, PTS, PUS1, PYGM, RAB23, RAG1, RAG2, RAPSN, RARS2, RDH12, RHO, RMRP, RPE65, RPGR, RIP1L, RPS19, RS1, RTEL1, SACS, SAMHD1, SBDA, SEPSECS, SERPINA1, SCNA, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMN1, SMPD1, STAR, SUMF1, TAT, TCIRG1, TECPR2, TFR2, TGFBR2, TGFBi, TGM1, TH, TIGIT, TIM3, TMEM216, TPP1, TRAC, TRMU, TSFM, TTPA, TYMP, USH1B, USH1C, USH2A, VLGR, VPS13A, VPS13B, VPS45, VRK1, VSX2, WAS, WNT10A, XPA, XPC, and ZFYVE26. In some instances, the gene is a safe harbor for gene integration. Such genes include albumin and transferrin. In some instances, the gene is a gene of a viral genome, including, but not limited to an HSV, HIV, HBV, influenza or coronavirus genome.

In some embodiments, the guide nucleic acid comprises a nucleotide sequence as described herein (e.g., TABLE 3, TABLE 4, TABLE 6, or TABLE 7). In some embodiments, a guide nucleic acid herein is at least about 70%, at least about 75%, at least about 80%, at least about 85%, at least about 90%, at least about 95%, at least about 98%, at least about 99% or 100% identical to a sequence selected from SEQ ID NOS: 202-207, 214-215, 224-229 and 231-232. In some embodiments, a guide nucleic acid is at least about 70%, at least about 75%, at least about 80%, at least about 85%, at least about 90%, at least about 95%, at least about 98%, at least about 99% or 100% identical to a sequence selected from SEQ ID NOS: 208-213. In some embodiments, a guide nucleic acid comprises a sequence that is at least about 70%, at least about 75%, at least about 80%, at least about 85%, at least about 90%, at least about 95%, at least about 98%, at least about 99% or 100% identical to UGGGGCAGUUGGUUGCCCUUAGCCUGAGGCAUUUAUUGCACUCGGGAAGUACCAUUUCU CAGAAAUGGUACAUCCAAC (SEQ ID NO: 214). In some embodiments, a guide nucleic acid comprises a sequence that is at least about 70%, at least about 75%, at least about 80%, at least about 85%, at least about 90%, at least about 95%, at least about 98%, at least about 99% or 100% identical to UGGUACAUCCAAC (SEQ ID NO: 215). In some embodiments, a guide nucleic acid comprises a sequence with at least 8, at least 9, at least 10, at least 11, at least 12, or at least 13 contiguous nucleotides of SEQ ID NO: 215. In some embodiments, a composition described herein can comprise a viral vector encoding CasM.19952 (SEQ ID NO: 23) and a guide nucleic acid comprising any one of SEQ ID NOs: 202-207, 214-215, 224-229 and 231-232. In some embodiments, a composition described herein can comprise a viral vector encoding CasM.19952 (SEQ ID NO: 23) and a guide nucleic acid comprising SEQ ID NOs: 208-213. Such nucleotide sequences described herein (e.g., TABLE 7 or TABLE 8) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a viral vector. Similarly, disclosure of the nucleotide sequences described herein (e.g., TABLE 7 or TABLE 8) also discloses the complementary nucleotide sequence, the reverse nucleotide sequence, and the reverse complement nucleotide sequence, any one of which can be a nucleotide sequence for use in a guide nucleic acid as described herein.

### VIII. Pharmaceutical Compositions

Disclosed herein, in some aspects, are pharmaceutical composition comprising a viral vector or a virus comprising a viral vector, wherein the viral vector comprises a transgene that encodes an effector protein and a guide nucleic acid; and a pharmaceutically acceptable excipient, carrier or diluent. In some instances, the transgene encodes an effector protein, effector protein modifications (e.g., nuclear localization signal (NLS), polyA tail), a guide nucleic acid, respective promoter(s), a donor nucleic acid, or a combination thereof. The viral vector may be a viral vector described herein, e.g., an AAV vector or an scAAV vector. The virus may be an adeno-associated virus. The effector protein may be an effector protein described herein. The guide nucleic acid may be a guide nucleic acid described herein. The donor nucleic acid may be a donor nucleic acid described herein. Non-limiting examples of pharmaceutically acceptable carriers and diluents include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); and preservatives.

In some embodiments, a pharmaceutically acceptable excipient, carrier or diluent is any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

### IX. Methods of Nucleic Acid Editing

Provided herein are methods of editing target nucleic acids using viral vectors as described herein. In general, editing refers to modifying the nucleobase sequence of a target nucleic acid. However, compositions disclosed herein may also be capable of making epigenetic modifications of target nucleic acids. Effector proteins, multimeric complexes thereof and systems described herein may be used for editing or modifying a target nucleic acid. Editing a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

Methods of editing may introduce a mutation (e.g., point mutations, deletions) in a target nucleic acid relative to a corresponding wildtype nucleobase sequence. Editing may remove or correct a disease-causing mutation in a nucleic acid sequence to produce a corresponding wildtype nucleobase sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some instances, cleavage (single-stranded or double-stranded) is site-specific, meaning cleavage occurs at a specific site in the target nucleic acid, often within the region of the target nucleic acid that hybridizes with the guide nucleic acid spacer sequence. In some cases, effector proteins introduce a single-stranded break in a target nucleic acid to produce a cleaved nucleic acid. In some cases, the effector protein introduces a break in a single stranded RNA (ssRNA). The effector proteins may be coupled to a guide nucleic acid that targets a particular region of interest in the ssRNA. In some instances, the target nucleic acid, and the resulting cleaved nucleic acid is contacted with a nucleic acid for homologous recombination (e.g., homology directed repair (HDR)) or non-homologous end joining (NHEJ). In some cases, a double-stranded break in the target nucleic acid may be repaired (e.g., by NHEJ or HDR) without insertion of a donor nucleic acid, such that the repair results in an indel in the target nucleic acid at or near the site of the double-stranded break.

In some instances, the effector protein is fused to a chromatin-modifying enzyme. In some cases, the fusion protein chemically modifies the target nucleic acid, for example by methylating, demethylating, or acetylating the target nucleic acid in a sequence specific or non-specific manner.

In some instances, editing a target nucleic acid comprises genome editing. Genome editing may comprise modifying a genome, chromosome, plasmid, or other genetic material of a cell or organism. In some instances, the genome, chromosome, plasmid, or other genetic material of the cell or organism is modified in vivo. In some instances, the genome, chromosome, plasmid, or other genetic material of the cell or organism is modified in a cell. In some instances, the genome, chromosome, plasmid, or other genetic material of the cell or organism is modified in vitro. For example, a plasmid may be modified in vitro using a composition described herein and introduced into a cell or organism. In some instances, modifying a target nucleic acid may comprise deleting a sequence from a target nucleic acid. For example, a mutated sequence or a sequence associated with a disease may be removed from a target nucleic acid. In some instances, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some instances, modifying a target nucleic acid may comprise introducing a sequence into a target nucleic acid. For example, a beneficial sequence or a sequence that may reduce or eliminate a disease may be inserted into the target nucleic acid.

In some instances, methods comprise inserting a donor nucleic acid into a cleaved target nucleic acid. The donor nucleic acid may be inserted at a specified (e.g., effector protein targeted) point within the target nucleic acid. In some instances, methods comprise contacting a target nucleic acid with a first effector protein thereby introducing a single-stranded break in the target nucleic acid; contacting the target nucleic acid with a second effector protein to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

### X. Methods of Treatment

Disclosed herein, in some aspects, are methods of administering a composition described herein to a subject in need thereof. Also disclosed herein, are methods of administering a cell or a population of cells comprising a composition described herein to a subject in need thereof. The subject may be a mammal. The subject may be a non-human subject. The subject may be a human subject. Methods of administering a composition or cell to a subject may be carried out in various manners, including aerosol inhalation, injection, transfusion, and implantation. The compositions and cells described herein may be administered to a subject intravenously, subcutaneously, intradermally, intratumorally, intramuscularly, or intraperitoneally. In some instances, compositions comprising viruses disclosed herein are administered to a subject via intravenous, parenteral, or subcutaneous injection.

In some instances, methods comprise administering a composition or cell described herein to a subject having cancer. The cancer may be a solid cancer (tumor). The cancer may be a blood cell cancer, including leukemias and lymphomas. Non-limiting types of cancer that could be treated with such methods and compositions include acute lymphoblastic lymphoma; adrenocortical carcinoma; anal cancer; appendix cancer; astrocytoma; atypical teratoid/rhabdoid tumor; basal-cell carcinoma; bile duct cancer; bone osteosarcoma; brain cancer; brain tumor; brainstem glioma; bronchial adenoma, carcinoid, or tumor; Burkitt lymphoma; carcinoma; colon cancer, colorectal cancer; emphysema; endometrial cancer; Ewing sarcoma; gallbladder cancer; gastric (stomach) cancer; gastrointestinal tumor; hypopharyngeal cancer; Kaposi Sarcoma; kidney cancer lip and oral cavity cancer; liposarcoma; non-small cell lung cancer; lymphoma; Waldenström; mesothelioma, myeloma; nasopharyngeal carcinoma; neuroblastoma; non-Hodgkin's lymphoma; pineal cancer; pituitary tumor; prostate cancer; rectal cancer, renal-cell carcinoma, retinoblastoma; spinal cord tumor; squamous cell carcinoma; squamous neck cancer; T-cell lymphoma, cutaneous (Mycosis Fungoides and Seźary syndrome); throat cancer; urethral cancer; vaginal cancer; and Wilms Tumor, liver cancer, bladder cancer, cancer of the kidney or ureter, lung cancer, cancer of the small intestine, esophageal cancer, melanoma, bone cancer, pancreatic cancer, skin cancer, brain cancer (e.g., glioblastoma), cancer of the head or neck, uterine cancer, ovarian cancer, breast cancer, testicular cancer, cervical cancer, stomach cancer, Hodgkin's Disease, non-Hodgkin's lymphoma, thyroid cancer. The cancer may be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), gliomahairy cell leukemia, myelogenous leukemia, myeloid leukemia; and chronic lymphocytic leukemia (CLL).

In some instances, methods comprise administering a composition or cell described herein to a subject having an infection caused by a pathogen, wherein the composition, or RNA(s) and/or protein(s) encoded by the composition, modifies a target nucleic acid of the pathogen. Non-limiting examples of pathogens are bacteria, a virus and a fungus. The target nucleic acid, in some cases, is a portion of a nucleic acid from a sexually transmitted infection or a contagious disease. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target region is a portion of a gene locus of bacterium or other pathogen responsible for a disease, wherein the gene locus comprises a mutation that confers resistance to a treatment, such as antibiotic treatment.

### Mutations

In some instances, methods comprise administering a composition or cell described herein to a subject, wherein the composition, or RNA(s) and/or protein(s) encoded by the composition, modifies a target nucleic acid, wherein the target nucleic acid comprises at least one mutation. In some instances, the composition modifies the sequence of the target nucleic acid having the mutation to a wildtype sequence. Non-limiting examples of mutations are insertion-deletion (indel), single nucleotide polymorphism (SNP), and frameshift mutations. The mutation may be a deletion of one or more nucleotides. In some instances, guide nucleic acids described herein hybridize to a region of the target nucleic acid comprising the mutation. The mutation may be located in a non-coding region or a coding region of a gene. Mutations may be associated with a phenotype of the organism that is altered from a wild type phenotype.

In some embodiments, target nucleic acids comprise a mutation. In some embodiments, a composition, system or method described herein can be used to modify a target nucleic acid comprising a mutation such that the mutation is modified to be a wild-type nucleotide or nucleotide sequence. In some embodiments, a composition, system or method described herein can be used to detect a target nucleic acid comprising a mutation.

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some embodiments, mutations comprise a point mutation, a chromosomal mutation, a copy number mutation, or any combination thereof. A point mutation may be a substitution, insertion, or deletion of a single nucleotide. In some embodiments, mutations comprise a chromosomal mutation. A chromosomal mutation may comprise an inversion, a deletion, a duplication, or a translocation of one or more nucleotides. In some embodiments, mutations comprise a copy number variation. A copy number variation may comprise a gene amplification or an expanding trinucleotide repeat. In some embodiments, guide nucleic acids described herein hybridize to a target region of a target nucleic acid comprising the mutation. In some embodiments, mutations are located in a non-coding region of a gene.

In some embodiments, target nucleic acids described herein comprise a mutation, wherein the mutation is a single nucleotide polymorphism (SNP). The SNP may be associated with a phenotype of the sample or a phenotype of the organism from which the sample was taken. A SNP, in some embodiments, is associated with an altered phenotype as compared to a wild-type phenotype. In some embodiments, a single nucleotide mutation, SNP, or deletion described herein is associated with a disease, such as a genetic disease. In some embodiments, the genetic disease is a disease, disorder, condition, or syndrome associated with or caused by one or more mutations in the DNA of an organism having the genetic disease. A SNP may be a synonymous substitution or a nonsynonymous substitution. A nonsynonymous substitution can be a missense substitution, or a nonsense point mutation. A synonymous substitution may be a silent substitution.

In some embodiments, the target nucleic acid comprises a mutation associated with a disease. In some instances, mutations are associated with a disease, that is the mutation in a subject indicates that the subject is susceptible to, or suffers from, a disease, disorder, condition, syndrome, or pathological state. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. Non-limiting examples of diseases associated with mutations are hemophilia, sickle cell anemia, β-thalassemia, Duchene muscular dystrophy, severe combined immunodeficiency (SCID, also known as “bubble boy syndrome”), Huntington's disease, cystic fibrosis, and various cancers.

In some embodiments, a target nucleic acid comprises a mutation of 1, 2, 3, 4, 5, 6, 7, 8, 9, 10, 11, 12, 13, 14, 15, 16, 17, 18, 19, 20 or more nucleotides. In some instances, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. The mutation may comprise a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may comprise a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1to 50, 1to 100, 25 to 50, 25 to 100, 50 to 100, 100 to 500, 100 to 1000, or 500 to 1000 nucleotides.

### Specific Targets and Indications

Described herein are compositions and methods for editing and/or detecting a target nucleic acid, wherein the target nucleic acid is a gene, a portion thereof, a transcript thereof. In some embodiments, the target nucleic acid is a reverse transcript (e.g. a cDNA) of an mRNA transcribed from the gene, or an amplicon thereof. In some embodiments, the target nucleic acid is an amplicon of at least a portion of a gene. In some embodiments, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be a RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by a reverse transcriptase. In some embodiments, the target nucleic acid is single-stranded RNA (ssRNA) or mRNA. In some embodiments, the target nucleic acid is from a virus, a parasite, or a bacterium described herein. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

In certain embodiments, the target nucleic acid is a double stranded nucleic acid comprising a target strand and a non-target strand, wherein the target strand comprises a target sequence. In some embodiments, where a target strand comprises a target sequence, at least a portion of the guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the guide nucleic acid is complementary to the target sequence on the target strand. In some embodiments, a target nucleic acid comprises a PAM that is located on the non-target strand. Such a PAM, in some embodiments, is adjacent (e.g., within 1, 2, 3, 4 or 5 nucleotides) to the 5′ end of the target sequence on the non-target strand of the double stranded DNA molecule. In certain embodiments, such a PAM is directly adjacent to the 5′ end of a target sequence on the non-target strand of the double stranded DNA molecule.

In some embodiments, an effector protein described herein or a multimeric complex thereof recognizes a PAM on a target nucleic acid. In some embodiments, multiple effector proteins of the multimeric complex recognize a PAM on a target nucleic acid. In some embodiments, only one effector protein of the multimeric complex recognizes a PAM on a target nucleic acid. In some embodiments, the PAM is 3′ to the spacer region of the crRNA. In some embodiments, the PAM is directly 3′ to the spacer region of the crRNA.

An effector protein of the present disclosure, a dimer thereof, or a multimeric complex thereof may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a sequence that is complementary to a guide nucleic acid spacer region.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, the target nucleic acid comprises a target locus. In certain embodiments, the target nucleic acid comprises more than one target loci. In some embodiments, the target nucleic acid comprises two target loci. Accordingly, in some embodiments, the target nucleic acid can comprise one or more target sequences.

In some embodiments, the target sequence covers the junction of two exons. In some embodiments, the target sequence is located within about 1 to about 300 nucleotides, about 10 to about 250, about 20 to about 200, about 30 to about 150, about 40 to about 100, or about 50 nucleotides of the 5′ untranslated region (UTR). In some embodiments, the target sequence is located within about 1 to about 300 nucleotides, about 10 to about 250, about 20 to about 200, about 30 to about 150, about 40 to about 100, or about 50 nucleotides of the 3′ UTR.

In some embodiments, the start of an exon is referred to interchangeably herein as the 5′ end of an exon. In certain embodiments, the 5′ region of an exon comprises a sequence about 1 to about 300 nucleotides adjacent to the 5′ end of an exon when moving upstream in the 5′ direction, or a sequence about 1 to about 300 nucleotides adjacent to the 5′ end of an exon when moving downstream in the 3′ direction, or both.

In some embodiments, the end of an exon is referred to interchangeably herein as the 3′ end of an exon. In certain embodiments, the 3′ region of an exon comprises a sequence about 1 to about 300 nucleotides adjacent to the 3′ end of an exon when moving upstream in the 5′ direction, or a sequence about 1 to about 300 nucleotides adjacent to the 3′ end of an exon when moving downstream in the 3′ direction, or both.

Nucleic acids, such as DNA and pre-mRNA, can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In certain embodiments, nucleic acids can contain one or more elements that act as a signal during transcription, splicing, and/or translation. In certain embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs).

In some embodiments, a target sequence that a guide nucleic acid binds is at least partially within a targeted exon, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds can comprise a sequence about 1 to about 300 nucleotides, about 10 to about 250, about 20 to about 200, about 30 to about 150, about 40 to about 100, or about 50 nucleotides adjacent to: the start of a targeted exon, the end of a targeted exon, or both.

In some embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: the start of a targeted exon, the end of a targeted exon, or both.

In some embodiments, a target sequence that a guide nucleic acid binds is at least partially within a targeted exon, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds can comprise a sequence about 1 to about 300 nucleotides, about 10 to about 250, about 20 to about 200, about 30 to about 150, about 40 to about 100, or about 50 nucleotides adjacent to: one or more signaling element comprising a 5′SS, a 3′SS, a premature stop codon, U1 binding sequence, U2 binding sequence, a BS, a PYT, ESE, an ISE, an ESS, an ISS, more than one of the foregoing, or any combination thereof.

In certain embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: one or more signaling element comprising a 5′SS, a 3′SS, a premature stop codon, U1 binding sequence, U2 binding sequence, a BS, a PYT, ESE, an ISE, an ESS, an ISS, more than one of the foregoing, or any combination thereof.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

In some embodiments, the target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are: AAVS1, ABCA4, ABCB11, ABCC8, ABCD1, ABCG5, ABCG8, ACAD9, ACADM, ACADVL, ACAT1, ACOX1, ACSF3, ADA, ADAMTS2, ADGRG1, AGA, AGL, AGPS, AGXT, AHI1, AIRE, ALDH3A2, ALDOB, ALG6, ALK, ALKBH5, ALMS1, ALPL, AMRC9, AMT, ANAPC10, ANAPC11, ANGPTL3, APC, Apo(a), APOCIII, APOEε4, APOL1, APP, AQP2, AR, ARFRP1, ARG1, ARH, ARL13B, ARL6, ARSA, ARSB, ASL, ASNS, ASPA, ASS1, ATM, ATP6V1B1, ATP7A, ATP7B, ATRX, ATXN1, ATXN10, ATXN2, ATXN3, ATXN7, ATXN8OS, AXIN1, AXIN2, B2M, BACE-1, BAK1, BAP1, BARD1, BAX2, BBS1, BBS10, BBS12, BBS2, BCKDHA, BCKDHB, BCL2L2, BCS1L, BEST1, Betaglobin gene, BLM, BMPR1A, BRAF, BRAFV600E, BRCA1, BRCA2, BRIP1, BSND, C9orf72, CA4, CACNA1A, CAH1, CAPN3, CASR, CBS, CCNB1, CC2D2A, CCR5, CD1, CD2, CD3, CD3D, CD3Z, CD4, CD5, CD6, CD7, CD8A, CD8B, CD9, CD14, CD18, CD19, CD21, CD22, CD23, CD27, CD28, CD30, CD33, CD34, CD36, CD38, CD40, CD40L, CD44, CD46, CD47, CD48, CD52, CD55, CD57, CD58, CD59, CD68, CD69, CD72, CD73, CD74, CD79A, CD80, CD81, CD83, CD84, CD86, CD90, CD93, CD96, CD99, CD100, CD123, CD160, CD163, CD164, CD164L2, CD166, CD200, CD204, CD207, CD209, CD226, CD244, CD247, CD274, CD276, CD300, CD320, CDC73, CDH1, CDH23, CDK11, CDK4, CDKN1A, CDKN1B, CDKN1C, CDKN2A, CDKN2B, CEBPA, CELA3B, CEP290, CERKL, CFB, CFTR, CHCHD10, CHEK2, CHM, CHRNE, CIITA, CLN3, CLN5, CLN6, CLN8, CLRN1, CLTA, CMT1A, CNBP, CNGB1, CNGB3, COL1A1, COL1A2, COL27A1, COL4A3, COL4A4, COL4A5, COL7A1, CPS1, CPT1A, CPT2, CRB1, CREBBP, CRX, CRYAA, CTNNA1, CTNNB1, CTNND2, CTNS, CTSK, CXCL12, CYBA, CYBB, CYP11B1, CYP11B2, CYP17A1, CYP19A1, CYP21A2, CYP27A1, DBT, DCC, DCLRE1C, DERL2, DFNA36, DFNB31, DGAT2, DHCR7, DHDDS, DICER1, DIS3L2, DLD, DMD, DMPK, DNAH5, DNAI1, DNAI2, DNM2, DNMT1, DPC4, DYSF, EDA, EDN3, EDNRB, EGFR, EIF2B5, EMC2, EMC3, EMD, EMX1, EN1, EPCAM, ERCC6, ERCC8, ESCO2, ETFA, ETFDH, ETHE1, EVC, EVC2, EYS, F5, F8, F9, FXI, FAH, FAM161A, FANCA, FANCB, FANCC, FANCD1, FANCD2, FANCE, FANCF, FANCG, FANCI, FANCJ, FANCL, FANCM, FANCN, FANCP, FANCS, FBN1, FGF14, FGFR2, FGFR3, FGA, FGB, FGG, FH, FHL1, FIX, FKRP, FKTN, FLCN, FMR1, FOXP3, FSCN2, FSHD1, FUS, FUT8, FVIII, FXII, FXN, G6PC, GAA, GALC, GALK1, GALT, GAMT, GATA2, GATA-4, GBA, GBE1, GCDH, GCGR, GDNF, GFAP, GFM1, GHR, GJB1, GJB2, GLA, GLB1, GLDC, GLE1, GNE, GNPTAB, GNPTG, GNS, GPC3, GPR98, GREM1, GRHPR, GRIN2B, H2AFX H2AX, HADHA, HAX1, HBA1, HBA2, HBB, HER2, HEXA, HEXB, HFE, HGSNAT, HLCS, HMGCL, HOGA1, HOXB13, HPRPF3, HPRT, HPS1, HPS3, HRAS, HRD1, HSD17B4, HSD3B2, HTT, HUS1, HYAL1, HYLS1, IDS, IDUA, IFITM5, IKBKAP, IL2RG, IL7R, IMPDH1, INPP5E, IRF4, ITGB2, ITPR1, IVD, JAG1, JAK1, JAK3, KCNC3, KCND3, KCNJ11, KLHL7, KRAS, LAMA2, LAMA3, LAMB3, LAMC2, LCA5, LDLR, LDLRAP1, LHX3, LIFR, LIPA, LMNA, LOR, LOXHD1, LPL, LRAT, LRP6, LRPPRC, LRRK2, MADR2, MAN2B1, MAPT, MAX, MCM6, MCOLN1, MECP2, MED17, MEFV, MEN1, MERTK, MESP2, MET, METex14, MFN2, MFSD8, MIA3, MITF, MKL2, MKS1, MLC1, MLH1, MLH3, MMJAA, MMAB, MMACHC, MMADHC, MMD, MPI, MPL, MPV17, MSH2, MSH3, MSH6, MTHFD1L, MTHFR, MTM1, MTRR, MTTP, MUT, MUTYH, MYC, MYH7, MYO7A, NAGLU, NAGS, NBN, NDRG1, NDUFAF5, NDUFS6, NEB, NF1, NF2, NKX2-5, NOG, NOTCH1, NOTCH2, NPC1, NPC2, NPHP1, NPHS1, NPHS2, NRAS, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9, PD1, PDCD1, PDE6B, PDGFRA, PDHA1, PDHB, PEX1, PEX10, PEX12, PEX13, PEX14, PEX16, PEX19, PEX2, PEX26, PEX3, PEX5, PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE, POMGNT1, POT1, POU5F1, PPM1A, PPP2R2B, PPT1, PRCD, PRKAG2, PRKAR1A, PRKCG, PRNP, PROM1, PROP1, PRPF31, PRPF8, PRPH2, PRPS1, PSAP, PSD95, PSEN1, PSEN2, PSRC1, PTCH1, PTEN, PTS, PUS1, PYGM RAB23, RAD50, RAD51C, RAD51D, RAG1, RAG2, RAPSN, RARS2, RB1, RDH12, RECQL4, RET, RHO, RICTOR, RMRP, ROS1, RP1, RP2, RPE65, RPGR, RPGRIP1L, RPL32P3, RS1, RTCA, RTEL1, RUNX1, SACS, SAMHD1, SCN1A, SCN2A, SDHA, SDHAF2, SDHB, SDHC, SDHD, SEL1L, SEPSECS, SERPINA1, SERPINC1, SERPING1, SGCA, SGCB, SGCG, SGSH, SIRT1, SLC12A3, SLC12A6, SLC17A5, SLC22A5, SLC25A13, SLC25A15, SLC26A2, SLC26A4, SLC35A3, SLC35B4 SLC37A4, SLC39A4, SLC4A11, SLC6A8, SLC7A7, SMAD3, SMAD4, SMAD7, SMARCA4, SMARCAL1, SMARCB1, SMARCE1, SMN1, SMPD1, SNAI2, SNCA, SNRNP200, SOD1, SOX10, SPARA7, SPTBN2, STAR, STAT3, STK11, SUFU, SUMF1, SYNE1, SYNE2, SYS1, TARDBP, TAT, TBK1, TBP, TCIRG1, TCTN3, TECPR2, TERC, TERT, TFR2, TGFBR2, TGM1, TH, TLE3, TMEM127, TMEM138, TMEM216, TMEM43, TMEM67, TMPRSS6, TOP1, TOPORS, TP53, TPP1, TRAC, TRMU, TSC1, TSC2, TSFM, TSPAN14, TTBK2, TTC8, TTPA, TTR, TULP1, TYMP, UBE2G2, UBE2J1, UBE3A, USH1C, USH1G, USH2A, VEGF, VHL, VPS13A, VPS13B, VPS35, VPS45, VRK1, VSX2, VWF, WAS, WDR19, WDR48, WNT10A, WRN, WS2B, WS2C, WT1, XPA, XPC, XPF, XRCC3, YAP1, ZAC1, ZEB1, ZFYVE26, and ZNF423. In some embodiments, the target sequence is at least partially within a targeted exon within any one of the genes described herein. A targeted exon can mean any portion within, contiguous with, or adjacent to a specified exon of interest can be targeted by the compositions, systems, and methods described herein. In some embodiments, one or more of the exons are targeted. In some embodiments, one or more of exons of any one the genes described herein are targeted. Nucleic acid sequences of target nucleic acids and/or corresponding genes are readily available in public databases as known and used in the art.

In some instances, the mutation is located in a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, D1S3L2, EGFR, EPCAM, FH, FLCN, GATA2, GPC3, GREM1, HOXB13, HRAS, KIT, MAX MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. In some instances, the mutation is associated with a blood disorder, e.g., a thalassemia or an anemia.

The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, ANGPTL3, AMT, Apo(a), ApoCIII, APOEε4, APP, AQP2, ARG1, ARSA, ARSB, ASL, ASNS, ASPA, ASS1, ATM, ATP6V1B1, ATP7A, ATP7B, ATRX, ATXN2, BACE-1, BBS1, BBS10, BBS12, BBS2, BCKDHA, BCKDHB, BCS1L, BLM, BSND, C90RF72, CAH1, CAPN3, CBS, CDH23, CEP290, CERKL, CHCHD10, CHM, CHRNE, CIITA, CLN3, CLN5, CLN6, CLN8, CLRN1, CMT1A, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, FSHD1, FUS, FVIII, FXI, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HTT, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MAPT, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PCSK9, PDHA1, PDHB, PEX1, PEX10, PEX12, PEX2, PEX6, PEX7, PFKM, PHGDH, PKHD1, PMP22, PMM2, POMGNT1, PPT1, PROP1, PRPS1, PSEN1, PSEN2, PSAP, PSD95, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, SOD1, SERPINC1, SERPING1, STAR, SUMF1, TARDBP, TAT, TCIRG1, TECPR2, TFR2, TGM1, TH, TMEM216, TPP1, TRMU, TSFM, TTR, TTPA, TYMP, USH1C, USH2A, VPS13A, VPS13B, VPS45, VRK1, VSX2, WNT10A, XPA, XPC, and ZFYVE26.

Described herein are compositions and methods for treating a disease in a subject by modifying a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include, but are not limited to: 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; acromegaly; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease (also called Alagille Syndrome); Alexander Disease, Alpers syndrome; alpha-1 antitrypsin deficiency (AATD); alpha-mannosidosis; Alstrom syndrome; Alzheimer's disease; amebic dysentery; amelogenesis imperfecta; amish type microcephaly; amyotrophic lateral sclerosis (ALS); anaplastic large cell lymphoma; anauxetic dysplasia; androgen insensitivity syndrome; angiopathic thrombosis; antiphospholipid syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant deafness; autosomal dominant polycystic kidney disease; autosomal recessive microtia; autosomal recessive renal glucosuria; autosomal visceral heterotaxy; babesiosis; balantidial dysentery; Bardet-Biedl syndrome; Bartter syndrome; basal cell nevus syndrome; Batten disease; benign recurrent intrahepatic cholestasis; beta-mannosidosis; β-thalassemia; Bethlem myopathy; Blackfan-Diamond anemia; bleeding disorder (coagulation); blepharophimosis; Byler disease; C syndrome; cachexia; CADASIL; calcific aortic stenosis; calcification of joints and arteries; carbamyl phosphate synthetase deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; Chaga's Disease; Charcot Marie Tooth Disease; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency 3; complement hyperactivation; complete androgen insentivity; cone rod dystrophies; conformational diseases; congenital adrenal hyperplasia; congenital bile acid synthesis defect type 1; congenital bile acid synthesis defect type 2; congenital defect in bile acid synthesis type; congenital erythropoietic porphyria; congenital generalized osteosclerosis; Cornelia de Lange syndrome; coronary heart disease; Cousin syndrome; Cowden disease; COX deficiency; Cri du chat syndrome; Crigler-Najjar disease; Crigler-Najjar syndrome type 1; Crisponi syndrome; Crouzon syndrome; Currarino syndrome; Curth-Macklin type ichthyosis hystrix; cutis laxa; cystic fibrosis; cystinosis; d-2-hydroxyglutaric aciduria; DDP syndrome; Dejerine-Sottas disease; Denys-Drash syndrome; Dercum disease; desmin cardiomyopathy; desmin myopathy; DGUOK-associated mitochondrial DNA depletion; diabetes Type I; diabetes Type II; disorders of glutamate metabolism; distal spinal muscular atrophy type 5; DNA repair diseases; dominant optic atrophy; Doyne honeycomb retinal dystrophy; Dravet Syndrome; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; encephalitis; enzymatic diseases; EPCAM-associated congenital tufting enteropathy; epidermolysis bullosa with pyloric atresia; epilepsy; fabry disease; facioscapulohumeral muscular dystrophy; Factor V Leiden thrombophilia; Faisalabad histiocytosis; familial atypical mycobacteriosis; familial capillary malformation-arteriovenous; Familial Creutzfeld-Jakob disease; familial esophageal achalasia; familial glomuvenous malformation; familial hemophagocytic lymphohistiocytosis; familial mediterranean fever; familial megacalyces; familial schwannomatosis; familial spina bifida; familial splenic asplenia/hypoplasia; familial thrombotic thrombocytopenic purpura; Fanconi disease (Fanconi anemia); Feingold syndrome; FENIB; fibrodysplasia ossificans progressiva; FKTN; Fragile X syndrome; Francois-Neetens fleck corneal dystrophy; Frasier syndrome; Friedreich's ataxia; FTDP-17; Fuchs corneal dystrophy; fucosidosis; G6PD deficiency; galactosialidosis; Galloway syndrome; Gardner syndrome; Gaucher disease; Gitelman syndrome; GLUT1 deficiency; GM2-Gangliosidoses (e.g., Tay Sachs Disease, Sandhoff Disease) glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; hairy cell leukemia; HANAC syndrome; harlequin type ichtyosis congenita; HDR syndrome; hearing loss; hemochromatosis type 3; hemochromatosis type 4; hemolytic anemia; hemolytic uremic syndrome; hemophilia A; hemophilia B; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; histiocytic sarcoma; HNF4A-associated hyperinsulinism; HNPCC; homozygous familial hypercholesterolemia; human immunodeficiency with microcephaly; human papilloma virus (HPV) infection; Huntington's disease; hyper-IgD syndrome; hyperinsulinism-hyperammonemia syndrome; hypercholesterolemia; hypertrophy of the retinal pigment epithelium; hypochondrogenesis; hypohidrotic ectodermal dysplasia; ICF syndrome; idiopathic congenital intestinal pseudo-obstruction; immunodeficiency 13; immunodeficiency 17; immunodeficiency 25; immunodeficiency with hyper-IgM type 1; immunodeficiency with hyper-IgM type 3; immunodeficiency with hyper-IgM type 4; immunodeficiency with hyper-IgM type 5; immunoglobulin alpha deficiency; inborn errors of thyroid metabolism; infantile myofibromatosis; infantile visceral myopathy; infantile X-linked spinal muscular atrophy; intrahepatic cholestasis of pregnancy; IPEX syndrome; IRAK4 deficiency; isolated congenital asplenia; Jeune syndrome; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Leber Congenital Amaurosis Teyp 10; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; leukocyte adhesion deficiency; Li Fraumeni syndrome; LIG4 syndrome; lipodystrophy; lissencephaly type 1; lissencephaly type 3; Loeys-Dietz syndrome; low phospholipid-associated cholelithiasis; Lynch Syndrome; lysinuric protein intolerance; a lysosomal storage disease (e.g., Hunter syndrome, Hurler syndrome); macular dystrophy; Maffucci syndrome; Majeed syndrome; mannose-binding protein deficiency; mantle cell lymphoma; Marfan disease; Marshall syndrome; MASA syndrome; mastocytosis; MCAD deficiency; McCune-Albright syndrome; MCKD2; Meckel syndrome; MECP2 Duplication Syndrome; Meesmann corneal dystrophy; megacystis-microcolon-intestinal hypoperistalsis; megaloblastic anemia type 1; MEHMO; MELAS; Melnick-Needles syndrome; MEN2s; meningitis; Menkes disease; metachromatic leukodystrophies; methymalonic acidemia due to transcobalamin receptor defect; methylmalonic acidurias; methylvalonic aciduria; microcoria-congenital nephrosis syndrome; microvillous atrophy; migraine; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; mycosis fungoides; myotonic dystrophy; NAIC; nail-patella syndrome; nemaline myopathies; neonatal diabetes mellitus; neonatal surfactant deficiency; nephronophtisis; Netherton disease; neurofibromatoses; neurofibromatosis type 1; neurofibromatosis type 2; Niemann-Pick disease type A; Niemann-Pick disease type B; Niemann-Pick disease type C; NKX2E; non-alcoholic fatty liver disease (NAFLD); non-alcoholic steatohepatitis (NASH); Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; osteogenesis imperfecta; otopalatodigital syndrome type 2; OXPHOS diseases; palmoplantar hyperkeratosis; panlobar nephroblastomatosis; Parkes-Weber syndrome; Parkinson's disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; platelet glycoprotein IV deficiency; pleuropulmonary blastoma and cystic nephroma; polycystic kidney disease; polycystic ovarian disease; polycystic lipomembranous osteodysplasia; Pompe disease, including infantile onset Pompe disease (IOPD) and late onset Pompe disease (LOPD); porphyrias; PRKAG2 cardiac syndrome, premature ovarian failure; primary erythermalgia; primary hemochromatoses; primary hyperoxaluria; progressive familial intrahepatic cholestasis; propionic acidemia; protein-losing enteropathy; pyruvate decarboxylase deficiency; RAPADILINO syndrome; renal cystinosis; retinitis pigmentosa; Rett Syndrome; rhabdoid tumor predisposition syndrome; Rieger syndrome; ring chromosome 4; Roberts syndrome; Robinow-Sorauf syndrome; Rothmund-Thomson syndrome; severe combined immunodeficiency disorder (SCID); Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; spinocerebellar ataxia; spinal muscular atrophy; split-hand/foot malformation with long bone deficiencies; spondylocostal dysostosis; sporadic visceral myopathy with inclusion bodies; storage diseases; Stargardt macular dystrophy; STRA6-associated syndrome; stroke; Tay-Sachs disease; thanatophoric dysplasia; thrombophilia due to antithrombin III deficiency; thyroid metabolism diseases; Tourette syndrome; transcarbamylase deficiency; transthyretin-associated amyloidosis; trisomy 13; trisomy 22; trisomy 2p syndrome; tuberous sclerosis; tufting enteropathy; urea cycle diseases; Usher Syndrome; Van Den Ende-Gupta syndrome; Van der Woude syndrome; variegated mosaic aneuploidy syndrome; VLCAD deficiency; von Hippel-Lindau disease; von Willebrand disease; Waardenburg syndrome; WAGR syndrome; Walker-Warburg syndrome; Werner syndrome; Wilson disease; Wiskott-Aldrich Syndrome; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease.

In some embodiments, compositions and methods modify at least one gene associated with the disease or the expression thereof. In some embodiments, the disease is Alzheimer's disease and the gene is selected from APP, BACE-1, PSD95, MAPT, PSEN1, PSEN2, and APOEε4. In some embodiments, the disease is Alzheimer's disease and the gene is APOE. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C90RF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease is congenital adrenal hyperplasia and the gene is CAH1. In some embodiments, the disease is congenital adrenal hyperplasia and the gene is CYP21A2. In some embodiments, the disease is cachexia and the gene is SMAD7. In some embodiments, the disease is hemophilia A and the gene is F8. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease is hypercholesterolemia and the gene is PCSK9. In some embodiments, the disease is hypercholesterolemia and the gene is ANGPTL3. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from DGAT2 and PNPLA3. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease is ornithine transcarbamylase deficiency and the gene is OTC. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot Marie Tooth disease and the gene is selected from PMP22 and MFN2. In some embodiments, the disease is Charcot Marie Tooth Type 2A and the gene is MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease is fabry disease and the gene is GLA. In some embodiments, the disease is facioscapulohumeral muscular dystrophy and the gene is FSHD1. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1. In some embodiments, the disease comprises histiocytosis and the gene is CDL. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease is neurofibromatosis type 2 and the gene is NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease is thrombophilia due to antithrombin III deficiency and the gene is SERPINC1. In some embodiments the disease is spinal muscular atrophy and the gene is SMN1. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX]O, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, the disease is cancer. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with cancer are ABL, ACE, AF4/HRX AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some instances, the oncogene is a gene that encodes a cyclin dependent kinase (CDK). Non-limiting examples of CDKs are CDK1, CDK4, CDK5, CDK7, CDK8, CDK9, CDK11 and CDK20. Non-limiting examples of tumor suppressor genes are TP53, RB1, and PTEN.

### Infections

Described herein are compositions and methods for treating an infection in a subject. Infections may be caused by a pathogen, e.g., bacteria, viruses, fungi, and parasites. Compositions and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, methods described herein include treating an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis virus A, Hepatitis virus B, Hepatitis virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, methods described herein include treating an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### Methods of Modifying Target Nucleic Acids

Disclosed herein are compositions, systems and methods comprising viral vectors for modifying a target nucleic acid. The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions, systems and methods comprising viral vectors reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions, systems and methods comprising viral vectors increase expression of one or more genes by at least 10%, at least 20%, at least 30%, at least 40%, at least 50%, at least 60%, at least 70%, at least 80%, at least 90%, or at least 100%.

In some instances, compositions, systems and methods use viral vectors encoding effector proteins that are fused to a heterologous protein. Heterologous proteins include, but are not limited to, transcriptional activators, transcriptional repressors, deaminases, methyltransferases, acetyltransferases, and other nucleic acid modifying proteins. In some cases, effector proteins need not be fused to a partner protein to accomplish the required protein (expression) modification.

An effector protein-guide nucleic acid complex may comprise high selectivity for a target sequence. In some embodiments, a ribonucleoprotein may comprise a selectivity of at least 200:1, 100:1, 50:1, 20:1, 10:1, or 5:1 for a target nucleic acid over a single nucleotide variant of the target nucleic acid. In some embodiments, a ribonucleoprotein may comprise a selectivity of at least 5:1 for a target nucleic acid over a single nucleotide variant of the target nucleic acid.

In some embodiments, compositions and methods comprise a nucleic acid expression vector, or use thereof, to introduce a Cas protein, guide nucleic acid, donor template or any combination thereof to a cell. In some embodiments, the nucleic acid expression vector is a viral vector and/or a plasmid. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. In some instances, compositions and methods provided herein comprise a lipid particle. In some embodiments, a lipid particle is a lipid nanoparticle (LNP). In some embodiments, a lipid or a lipid nanoparticle can encapsulate an expression vector. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some cases, a method can comprise contacting a cell with an expression vector. In some cases, contacting can comprise electroporation, lipofection, or lipid nanoparticle (LNP) delivery of an expression vector. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

### Administering Cells

In some instances, methods comprise administering a cell or a population of cells to a subject, wherein the cell or population of cells has been contacted with or modified by a composition disclosed herein. In general, the cell is a human cell. In some embodiments, the human cell is a: muscle cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells. In some instances, cells are administered to a subject by intravenous or parenteral injection. In some instances, cells are administered directly into a tumor, lymph node or site of infection.

In some instances, methods comprise performing leukapheresis on a subject, wherein leukocytes are collected, enriched, or depleted ex vivo to enrich T cells. The enriched T cells may be cultured to proliferate before contacting them with a composition described herein to produce autologous CAR T-cells. The autologous CAR T-cells may be administered to the subject less than about 1 week, less than about 2 weeks, less than about 3 weeks, less than about 4 weeks, less than about 5 weeks, or less than about 6 weeks from the time leukapheresis is completed. Cells described herein, including CAR-T cells, may be administered at a dosage of 10⁴ to 10⁹ cells/kg body weight. In some instances, methods comprise administering 10⁵ to 10⁶ cells/kg body weight.

### XI. Systems

Disclosed herein, in some aspects, are systems for detecting, modifying, or editing a target nucleic acid, comprising viral vectors encoding the effector proteins described herein, or a multimeric complex thereof. Systems may be used to detect, modify, or edit a target nucleic acid. Systems may be used to modify the activity or expression of a target nucleic acid. In some embodiments, systems comprise a viral vector encoding an effector protein described herein, a reagent, support medium, or a combination thereof.

In some embodiments, the effector protein comprises an effector protein, or a fusion protein thereof, described herein. In some embodiments, effector proteins comprise an amino acid sequence that is at least 65%, at least 70%, at least 75%, at least 80%, at least 85%, at least 90%, at least 95%, or 100% identical to any one of the sequences of TABLE 1. In some embodiments, the amino acid sequence of the effector protein is at least 65%, at least 70%, at least 75%, at least 80%, at least 85%, at least 90%, at least 95%, or 100% identical to any one of the sequences of TABLE 1.

In some embodiments, systems comprise viral vectors encoding an effector protein described herein, a guide nucleic acid described herein, a reagent, support medium, or a combination thereof. In some embodiments, the effector protein comprises an effector protein, or a fusion protein thereof, described herein. In some embodiments, effector protein comprises an amino acid sequence that is at least 65%, at least 70%, at least 75%, at least 80%, at least 85%, at least 90%, at least 95%, or 100% identical to any one of the sequences of TABLE 1. In some embodiments, the amino acid sequence of the effector protein is at least 65%, at least 70%, at least 75%, at least 80%, at least 85%, at least 90%, at least 95%, or 100% identical to any one ofthe sequences of TABLE 1. In some embodiments, the guide nucleic acid comprises a repeat sequence that is at least 65%, at least 70%, at least 75%, at least 80%, at least 85%, at least 90%, at least 92%, at least 95%, or 100% identical to any one of the sequences set forth in TABLE 3 and a spacer sequence that is at least 65%, at least 70%, at least 75%, at least 80%, at least 85%, at least 90%, at least 92%, at least 95%, or 100% identical to the spacer sequence set forth in TABLE 4.

Systems may be used for detecting the presence or the absence of a target nucleic acid as described herein. Systems may be used for detecting the presence or the absence of a target nucleic acid associated with or causative of a disease or disorder, such as a genetic disorder. Systems may be used for detecting the presence or the absence of a target nucleic acid associated with or causative of a disease or disorder as described herein. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

Reagents and viral vectors encoding effector proteins of various systems may be provided in a reagent chamber or on a support medium. Alternatively, the reagent and/or the viral vector encoding effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

### System Solutions

In general, systems comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises at least one salt. In some embodiments, the at least one salt is selected from potassium acetate, magnesium acetate, sodium chloride, potassium chloride, magnesium chloride, calcium chloride, and any combination thereof. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.010% (v/v) to 10% (v/v). In some embodiments, the concentration of the crowding agent in the solution is 0.5% (v/v) to 10% (v/v). In some embodiments, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), β-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. In some embodiments, a reporter nucleic acid comprises a non-target nucleic acid molecule that can provide a detectable signal upon cleavage by an effector protein. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a co-factor for an effector protein or a multimeric complex thereof may be assessed, such as by methods based on those described by Sundaresan et al. (Cell Rep. 2017 Dec. 26; 21(13): 3728-3739). In some embodiments, an effector or a multimeric complex thereof forms a complex with a co-factor. In some embodiments, the co-factor is a divalent metal ion. In some embodiments, the divalent metal ion is selected from Mg²⁺, Mn²⁺, Zn²⁺, Ca²⁺, Cu²⁺. In some embodiments, the divalent metal ion is Mg²⁺. In some embodiments, the co-factor is Mg²⁺.

### Reporters

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal. As used throughout the disclosure, a detectable signal is a signal that can be detected using optical, fluorescent, chemiluminescent, electrochemical or other detection methods known in the art. As used herein, “reporter” is used interchangeably with “reporter nucleic acid” or “reporter molecule”. The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some embodiments, reporters comprise a protein capable of generating a signal. A signal may be a calorimetric, potentiometric, amperometric, optical (e.g., fluorescent, colorimetric, etc.), or piezo-electric signal. In some embodiments, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin.

Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only deoxyribonucleotide residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified deoxyribonucleotides.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, compositions, systems and methods comprise viral vectors encoding an effector protein, and a reporter nucleic acid configured to undergo trans cleavage by the effector protein. Trans cleavage of the reporter may generate a signal from the reporter or alter a signal from the reporter. In some embodiments, the signal is an optical signal, such as a fluorescence signal or absorbance band. Trans cleavage of the reporter may alter the wavelength, intensity, or polarization ofthe optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid may be referred to as ‘DETECTR’. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Amplification Reagents Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, systems comprise a PCR tube, a PCR well or a PCR plate. The wells ofthe PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target region, an effector protein capable of being activated when complexed with the guide nucleic acid and the target region, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target region; an effector protein capable of being activated when complexed with the guide nucleic acid and the target region; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target region; and an effector protein capable of being activated when complexed with the guide nucleic acid and the target region. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target region; and an effector protein capable of being activated when complexed with the guide nucleic acid and the target region. The wells of the PCR plate may be pre-aliquoted with the guide nucleic acid targeting a target region, and an effector protein capable of being activated when complexed with the guide nucleic acid and the target region. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., or any value 20° C. to 45° C. In some embodiments, the nucleic acid amplification reaction is performed at a temperature of at least 20° C., 25° C., 30° C., 35° C., 37° C., 40° C., or 45° C., or any value 20° C. to 45° C. In some embodiments, the nucleic acid amplification reaction is performed at a temperature of 20° C. to 45° C., 25° C. to 40° C., 30° C. to 40° C., or 35° C. to 40° C.

Often, systems comprise primers for amplifying a target nucleic acid to produce an amplification product comprising the target nucleic acid and a PAM. For embodiment, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid, methods of assaying for a target nucleic acid that lacks a PAM by amplifying the target nucleic acid to introduce a PAM, and compositions used in introducing a PAM via amplification into the target nucleic acid.

### Additional System Components

In some embodiments, systems include a package, carrier, or container that is compartmentalized to receive one or more containers such as vials, tubes, and the like, each ofthe container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some embodiments, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines, 5 to 20 consecutive thymines, 5 to 20 consecutive cytosines, or 5 to 20 consecutive guanines. In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems are employed under certain conditions that enhance trans cleavage activity of an effector protein. In some embodiments, under certain conditions, transcollateral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some embodiments, the salt is NaCl. In some embodiments, the salt is KNO₃. In some embodiments, the salt concentration is less than 150 mM, less than 125 mM, less than 100 mM, less than 75 mM, less than 50 mM, or less than 25 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 50° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., or about 50° C.

### Examples

The following examples are included for illustrative purposes only and are not intended to limit the scope of the invention.

### Example 1. Aav Vector Encoding CasΦ12 and Guide RNAs Edit Pcsk9 in Mammalian Cells

This example demonstrates that genome editing can be performed with an AAV vector encoding a Cas effector having a length of between 700 and 800 amino acids (CasΦ12) and a guide RNA targeting PCSK9. Several crRNAs with varying repeat lengths of 36, 25, 20, or 19 nucleotides in combination with spacer lengths of 20, 17, or 16 nucleotides were tested. Each crRNA was cloned into an AAV vector with a U6 promoter to drive crRNA expression, and an intron-less EF1alpha short (EFS) promoter driving CasΦ12 expression. The AAV vector also included a polyA signal and 1 kb stuffer sequence. Hepal-6 mouse hepatoma cells were nucleofected with 10 μg of AAV plasmid. After 72 hours, genomic DNA was extracted and the frequency of indel mutations was determined using NGS.

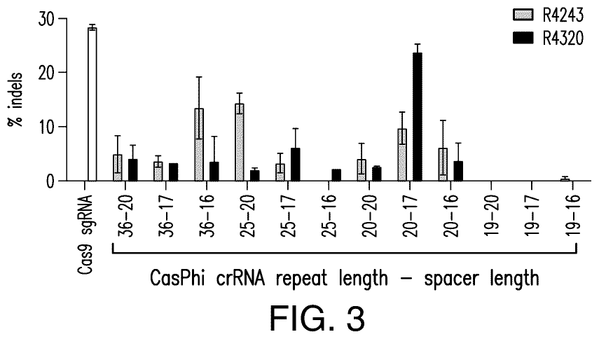

FIG. 3 shows the frequency of CasΦ12 induced indel mutations in Hepal-6 cells transduced with g of each AAV plasmid. In the graph legend, repeat and spacer lengths are indicated as the number of nucleotides in the repeat followed by the number of nucleotides in the spacer, e.g., 20-17 has a repeat length of 20 nucleotides and a spacer length of 17 nucleotides. The frequency of indel mutations is comparable to that of Cas9. This study demonstrates that a vector encoding a guide RNA and CasΦ12 provide robust genome editing across different gRNA sequences and with gRNAs of different repeat and spacer lengths.

### Example 2. Aav Vector Encoding Cas Effector, Guide RNA and Donor Nucleic Acid Restores Cftr Gene

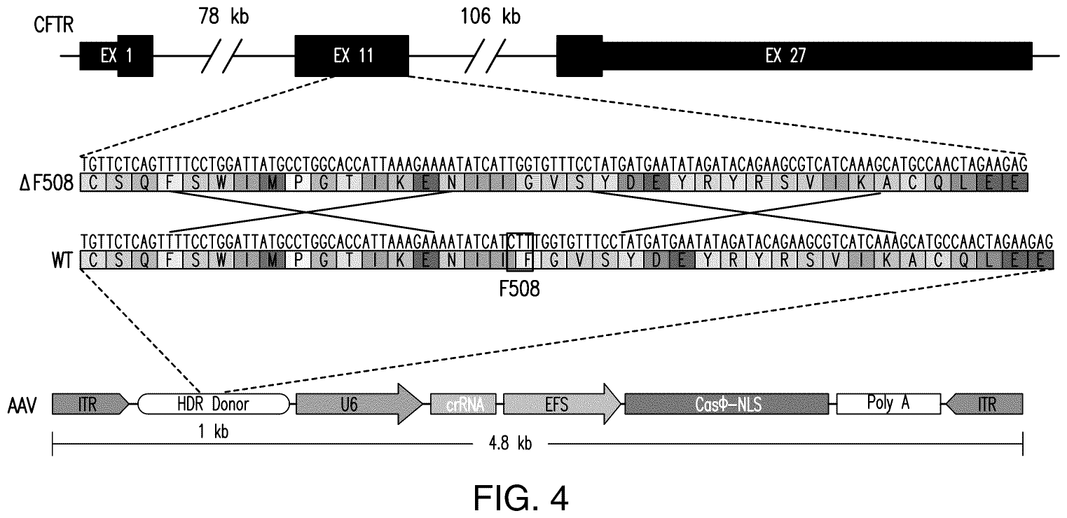

An AAV vector is constructed to contain a transgene between its ITRs, the transgene providing or encoding, in a 5′ to 3′ direction, a donor nucleic acid, a U6 promoter, a crRNA, an EFS promoter, a Cas effector protein having a nuclear localization signal, and a poly A tail, as shown in FIG. 4. The size of the donor nucleic acid is about 1 kb. The size of the Cas effector is less than 900 amino acids. The total length of the transgene, including the ITRs, is about 4.8 kb. The donor nucleic acid encodes a phenylalanine at the position that corresponds to amino acid 508 of a wildtype CFTR allele. The AAV vector is expressed with supporting plasmids to produce an adeno-associated virus (AAV). Lung epithelial cells comprising a mutation that results in deletion of phenylalanine at amino acid position 508 of CFTR (CFTRdelF508) mutation are contacted with the AAV. After about 48 hours, DNA or RNA is isolated from the infected cells. Insertion of the donor nucleic acid in the CFTR gene, and correction of the mutation, is confirmed by sequencing and/or Q-PCR.

### Example 3. Production of Car T-Cells with Aav Vector Encoding Cas Effector, Guide RNA, and Car

An AAV vector is constructed to contain a transgene between its ITRs, the transgene providing or encoding, in a 5′ to 3′ direction, a donor nucleic acid encoding a chimeric antigen receptor (CAR), a U6 promoter, a crRNA having a sequence complementary to an equal length portion of an HLA encoding sequence, an EFS promoter, a Cas effector protein having a nuclear localization signal, and a poly A tail. The size of the donor nucleic acid is about 1 kb. The size of the Cas effector is less than 900 amino acids. The total length of the transgene, including the ITRs, is about 4.8 kb. The AAV vector is expressed with supporting plasmids to produce an adeno-associated virus (AAV). T cells from a healthy donor subject are contacted with the AAV. After about 48 hours, DNA or RNA is isolated from the infected cells. Expression of the CAR and reduced expression of the HLA is confirmed by Q-PCR.

### Example 4. Gene Editing of Various Cell Types with Aav Vector Encoding Cas Effector, Guide RNA and Donor Nucleic Acid

An AAV vector is constructed to contain a transgene between its ITRs, the transgene providing or encoding, in a 5′ to 3′ direction, a donor nucleic acid, a U6 promoter, a crRNA, an EFS promoter, a Cas effector protein having a nuclear localization signal, and a poly A tail. The size of the donor nucleic acid is about 1 kb. The size of the Cas effector is less than 900 amino acids. The total length of the transgene, including the ITRs, is about 4.8 kb. The AAV vector is expressed with supporting plasmids to produce an adeno-associated virus (AAV). Hepatocytes, iPSCs or CD34+ hematopoietic stem cells (HSC) are contacted with the AAV. After about 48 hours, DNA or RNA is isolated from the infected cells. Insertion of the donor nucleic acid is confirmed by sequencing and/or Q-PCR.

### Example 5. Gene Editing of Various Cell Types with scAAV Vector Encoding Cas Effector and Guide RNA

An scAAV vector is constructed to contain a transgene between its ITRs, the transgene providing or encoding, in a 5′ to 3′ direction, a U6 promoter, a crRNA, an EFS promoter, a Cas effector protein having a nuclear localization signal, and a poly A tail. The size of the Cas effector is less than 500 amino acids. The total length of the transgene, including the ITRs, is about 2.5 kb. The AAV vector is expressed with supporting plasmids to produce an adeno-associated virus (AAV). Hepatocytes, iPSCs or CD34+ hematopoietic stem cells (HSC) are contacted with the AAV. After about 48 hours, DNA or RNA is isolated from the infected cells. An indel caused by the guide nucleic acid is confirmed by sequencing and/or Q-PCR.

### Example 6: Casm.19952 Edits Genomic DNA in Mammalian Cells

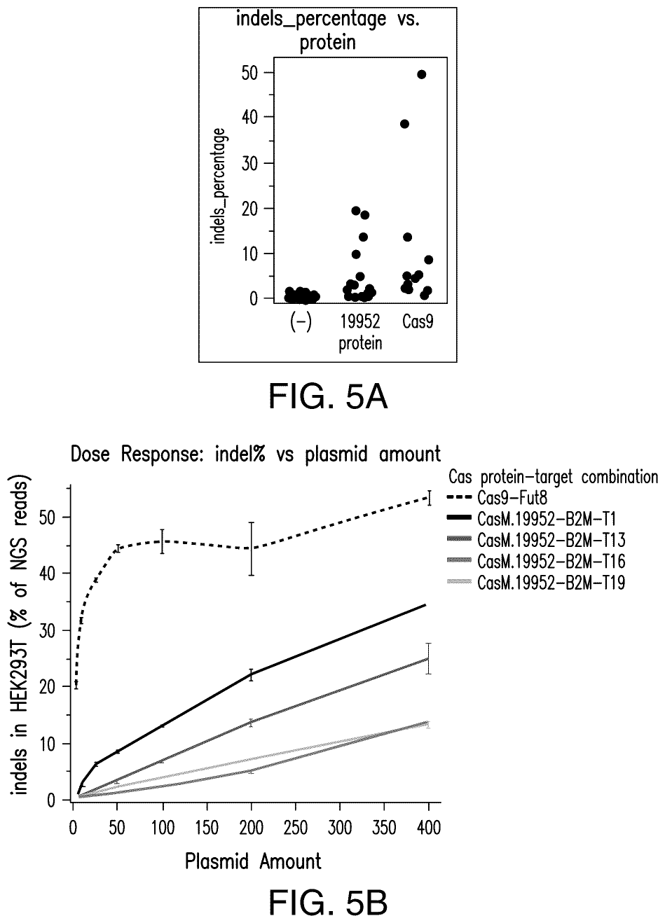

CasM.19952 was tested for its ability to produce indels in HEK293T cells. Briefly, a plasmid encoding CasM.19952 and a guide RNA was delivered by lipofection to HEK293T cells. This was performed for a variety of guide RNAs targeting up to twenty-four loci adjacent to biochemically determined PAM sequences. Indels were detected by next generation sequencing of PCR amplicons at the targeted loci and indel percentage was calculated as the fraction of sequencing reads containing insertions or deletions relative to an unedited reference sequence. Sequencing libraries with less than 20% of reads aligning to the reference sequence were excluded from the analysis for quality control purposes. “No plasmid” and SpyCas9 were included as negative and positive controls, respectively. FIG. 5A shows the results. TABLE 8 describes the sequences of the single guide RNAs tested that provided the greatest percent of reads with indels. Non-bold, non-italicized, capital letters indicate the tracrRNA region of the guide RNA; italicized letters indicate a linker; bold letters indicate the repeat sequence; and the lowercase letters represent the spacer sequence. This experiment demonstrated that CasM.19952 is a robust editor of genomic DNA in mammalian cells.

A dose-response experiment confirmed the genome editing capability of CasM.19952 in mammalian cells. Plasmids encoding CasM.19952 and single guide RNAs were delivered at various concentrations by lipofection into HEK293T. CasM.19952 was programmed to target four loci. SpyCas9 was included as a positive control. Indels were observed at all four loci. Results are shown in FIG. 5B.

TABLE 8
sgRNAs that provided genome editing with CasM.19952 in HEK293T cells

| sgRNA Sequence | | % of reads |
|---|---|---|
| DNA sequence | RNA sequence | with indels |
| TGGGGCAGTTGGTTGCCCTTAGCC | UGGGGCAGUUGGUUGCCCUUAGCC | 13.47 |
| TGAGGCATTTATTGCACTCGGGA | UGAGGCAUUUAUUGCACUCGGGA | |
| AGTACCATTTCTCAGAAATGGTAC | AGUACCAUUUCUCAGAAAUGGUAC | |
| ATCCAACtctaggcgcccgctaagttc | AUCCAACucuaggcgcccgcuaaguuc | |
| (SEQ ID NO: 208) | (SEQ ID NO: 202) | |
| TGGGGCAGTTGGTTGCCCTTAGCC | UGGGGCAGUUGGUUGCCCUUAGCC | 4.63 |
| TGAGGCATTTATTGCACTCGGGA | UGAGGCAUUUAUUGCACUCGGGA | |
| AGTACCATTTCTCAGAAATGGTAC | AGUACCAUUUCUCAGAAAUGGUAC | |
| ATCCAACcccgggtaagcctgtctgct | AUCCAACcccggguaagccugucugcu | |
| (SEQ ID NO: 209) | (SEQ ID NO: 203) | |
| TGGGGCAGTTGGTTGCCCTTAGCC | UGGGGCAGUUGGUUGCCCUUAGCC | 19.40 |
| TGAGGCATTTATTGCACTCGGGA | UGAGGCAUUUAUUGCACUCGGGA | |
| AGTACCATTTCTCAGAAATGGTAC | AGUACCAUUUCUCAGAAAUGGUAC | |
| ATCCAACcgtgctgtttcctccccacg | AUCCAACcgugcuguuuccuccccacg | |
| (SEQ ID NO: 210) | (SEQ ID NO: 204) | |
| TGGGGCAGTTGGTTGCCCTTAGCC | UGGGGCAGUUGGUUGCCCUUAGCC | 3.15 |
| TGAGGCATTTATTGCACTCGGGA | UGAGGCAUUUAUUGCACUCGGGA | |
| AGTACCATTTCTCAGAAATGGTAC | AGUACCAUUUCUCAGAAAUGGUAC | |
| ATCCAACgtgccttagtttcttcatct | AUCCAACgugccuuaguuucuucaucu | |
| (SEQ ID NO: 211) | (SEQ ID NO: 205) | |
| TGGGGCAGTTGGTTGCCCTTAGCC | UGGGGCAGUUGGUUGCCCUUAGCC | 18.35 |
| TGAGGCATTTATTGCACTCGGGA | UGAGGCAUUUAUUGCACUCGGGA | |
| AGTACCATTTCTCAGAAATGGTAC | AGUACCAUUUCUCAGAAAUGGUAC | |
| ATCCAACggggggggggggagaaaaa | AUCCAACggggggggggggagaaaaa | |
| (SEQ ID NO: 212) | (SEQ ID NO: 206) | |
| TGGGGCAGTTGGTTGCCCTTAGCC | UGGGGCAGUUGGUUGCCCUUAGCC | 9.48 |
| TGAGGCATTTATTGCACTCGGGA | UGAGGCAUUUAUUGCACUCGGGA | |
| AGTACCATTTCTCAGAAATGGTAC | AGUACCAUUUCUCAGAAAUGGUAC | |
| ATCCAACgcgccctccgatctggggtg | AUCCAACgcgcccuccgaucuggggug | |
| (SEQ ID NO: 213) | (SEQ ID NO: 207) | |

### Example 7: In Vivo Editing of PCSK9 in a Mammal Using AAV Vector Encoding CasΦ12 and Guide RNA

This example demonstrates that genome editing can be performed with an AAV8 vector encoding a CasΦ12 protein (SEQ ID NO: 57) and a guide RNA targeting PCSK9 in a mammal. An AAV8 vector was constructed to contain a transgene between its ITRs, the transgene providing or encoding, in a 5′ to 3′ direction, a donor nucleic acid, a U6 promoter, a crRNA, an EFS promoter, a Cas effector protein having a nuclear localization signal, and a poly A tail. The crRNA comprised a repeat sequence AUUGCUCCUUACGAGGAGAC (SEQ ID NO: 233) in combination with a spacer GAGCAACGGCGGAAGGU (SEQ ID NO: 234). The full crRNA had a sequence of GGAUUGCUCCUUACGAGGAGACGAGCAACGGCGGAAGGU (SEQ ID NO: 235). The AAV8 vector was expressed with supporting plasmids to produce an adeno-associated virus.

Four mice were injected with either PBS or 5e+11 of AAV virus. Mice liver samples were extracted either 14 days or 28 days post-injection and homogenized.

Genomic DNA was extracted from the homogenized liver samples and the frequency of indel mutations was determined using NGS. Serum PCSK9 (pg/ml) was determined.

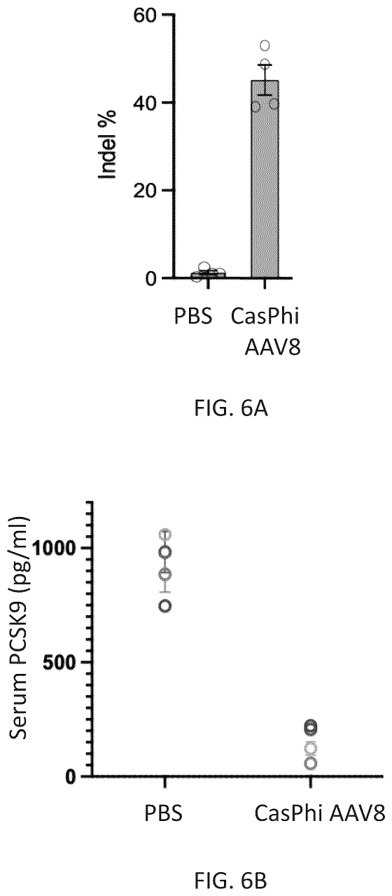

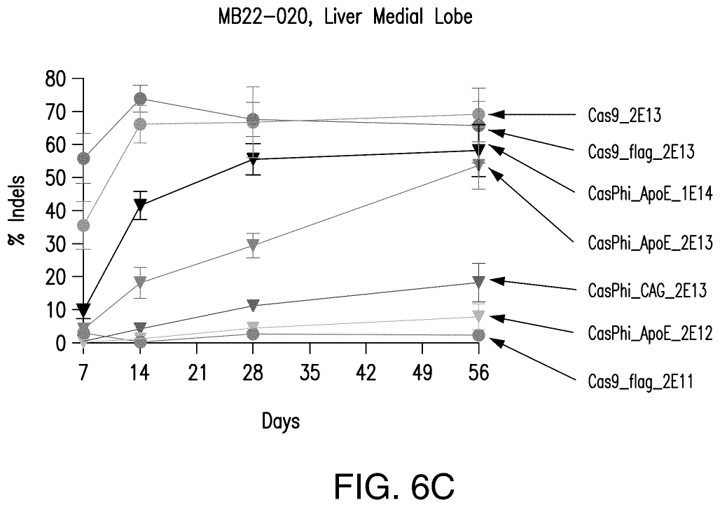

FIG. 6A illustrates the frequency of CasΦ12 induced indel mutations in mice liver transfected with 5e+11 of AAV plasmid. The results depicted in FIG. 6A demonstrate that CasΦ12 introduced about 45% indel mutations in a nucleotide sequence encoding PCSK9 protein. FIG. 6B illustrates that the serum PCSK9 (pg/ml) concentration is reduced in CasΦ12 injected mice. The results depicted in FIG. 6B demonstrate that the indel mutations resulted in 80% reduction in PCSK9 serum concentration.

This study demonstrates in vivo genome editing in mice liver using a vector encoding a guide RNA and CasΦ12.

A follow-up experiment was similarly performed, including the same guide nucleic acids, with varying viral doses (2E12 to 1E14) and using Cas9 as a control. In addition, an ApoE or CAG promoter was used to drive expression of CasPhi.12. A TBG promoter was used to drive expression of Cas9. The % indel was assayed via NGS at 7, 14, 28 and 56 days. Results are shown in FIG. 6C. Cas9 at 2E13 achieved ˜70% indels by week 2 and plateaued after that. 1E14 and 2E13 CasPhi.12 achieved ˜60% indels by week 8. These results demonstrate that an AAV encoding CasPhi.12 and a corresponding guide nucleic acid can achieve editing levels comparable with Cas9 in a mammalian liver in vivo.

### Example 8: In Vivo Gene Editing in a Mammal Using AAV Vector Encoding Cas Effector Protein

This example demonstrates that genome editing can be performed with an AAV vector encoding a Cas effector having any one of sequences recited in TABLE 1, and a guide RNA targeting a target sequence within a target nucleic acid. An AAV vector is constructed to contain a transgene between its ITRs, the transgene providing or encoding, in a 5′ to 3′ direction, a donor nucleic acid, a U6 promoter, a guide RNA, an EFS promoter, a Cas effector protein having a nuclear localization signal, and a poly A tail. The AAV vector is expressed with supporting plasmids to produce an adeno-associated virus.
mice are injected with 5e+11 of AAV virus. Mice liver samples are extracted either 14 days or 28 days post-injection.

Genomic DNA is extracted from homogenized liver samples and the frequency of indel mutations is determined using NGS.

### Example 9: AAV Vectors for Gene Editing by a Single Cut

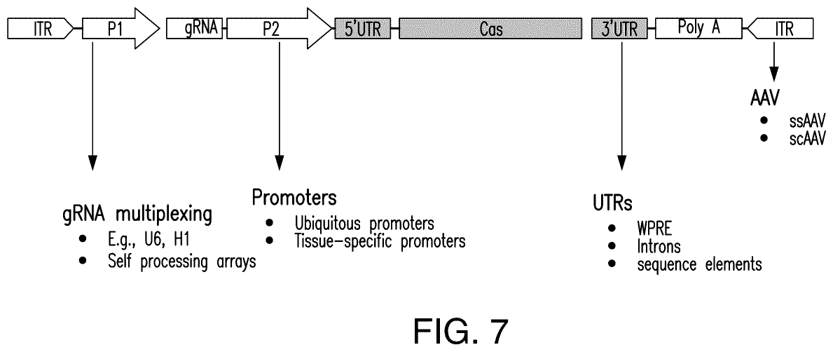

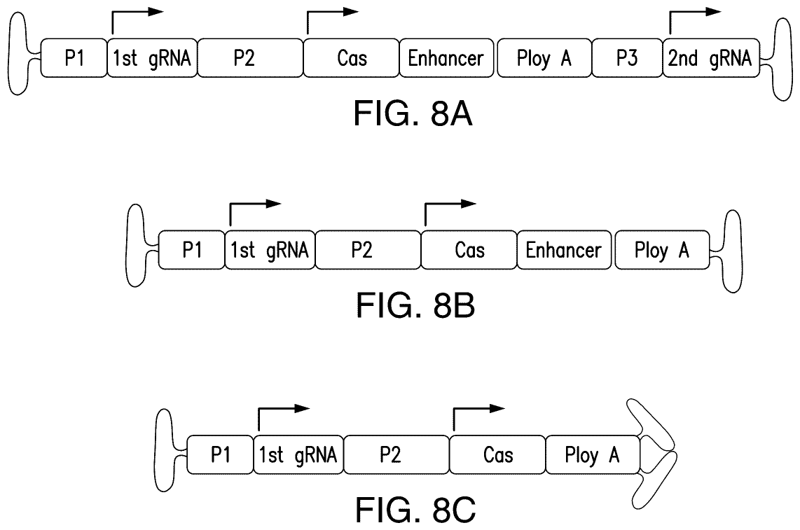

An AAV vector is constructed to contain a transgene between its ITRs. The transgene provides or encodes, in a 5′ to 3′ direction, a first promoter (P1), a guide nucleic acid, a second promoter (P2), an effector protein, and a poly A signal, as illustrated in FIG. 7. Optionally, the AAV vector comprises additional promoters, guide nucleic acids, transcriptional enhancers (e.g., WPRE), or a combination thereof, as illustrated in FIGS. 8A-8C. The effector protein has an amino acid sequence that has at least 80% identity to a sequence recited in TABLE 1. The guide nucleic acid comprises a sequence that has at least 90% identity to any one of sequences recited in TABLE 7. As illustrated in FIG. 7, the effector protein can be expressed either ubiquitously or tissue-specifically based on the second promoter the AAV vector is engineered to have. Each of the promoters is independently selected from ApoE, TBG, CMV, 7SK, EF1a, RPBSA, hPGK, EFS, SV40, PGK1, hPGK, Ube, human beta actin promoter, CAG, TRE, UAS, Ac5, Polyhedrin, CaMKIIa, GAL1-10, H1, TEF1, GDS, ADH1, CaMV35S, Ubi, U6, MNDU3, Ck8e, SPC5-12, Desmin, MND, and MSCV. The Poly A signal sequence can be either hGH Poly A signal sequence or sv40 Poly A signal sequence. The AAV vector is expressed with supporting plasmids to produce an adeno-associated virus (AAV).

### Example 10: AAV Vectors for Gene Editing by a Dual Cut

An AAV vector is constructed to contain a transgene between its ITRs. The transgene provides or encodes, in a 5′ to 3′ direction, first promoter (P1), a first guide nucleic acid, a second promoter (P2), an effector protein, an enhancer, a poly A signal, a third promoter (P3), and a second guide nucleic acid as illustrated in FIG. 8A, wherein the first guide nucleic acid and the second guide nucleic acid comprise different spacer sequences targeting different sequences in a target nucleic acid. In some instances, the different sequences are located on either side of an exon. Thus, contact of both guide nucleic acids to the target nucleic acid results in an exon deletion. The effector protein has an amino acid sequence that has at least 80% identity to a sequence recited in TABLE 1. The first guide nucleic acid and the second guide nucleic acid independently comprises a sequence that has at least 90% identity to any one of sequences recited in TABLE 7. Each of the promoters is independently selected from ApoE, TBG, CMV, 7SK, EF1a, RPBSA, hPGK, EFS, SV40, PGK1, hPGK, Ube, human beta actin promoter, CAG, TRE, UAS, Ac5, Polyhedrin, CaMKIIa, GAL1-10, H1, TEF1, GDS, ADH1, CaMV35S, Ubi, U6, MNDU3, Ck8e, SPC5-12, Desmin, MND, and MSCV. Also, as illustrated in FIG. 7, the effector protein can be expressed either ubiquitously or tissue-specifically based on the second promoter the AAV vector is engineered to have. The AAV vector is expressed with supporting plasmids to produce an adeno-associated virus (AAV).

### Example 11: AAV Vectors for Gene Insertion

An AAV vector is constructed to contain a transgene between its ITRs. The transgene provides or encodes, in a 5′ to 3′ direction, a first promoter (P1), a first guide nucleic acid, a second promoter (P2), an effector protein, an enhancer, and a poly A signal sequence. The combination of guide nucleic acid and the effector protein are designed for cleaving within, directly adjacent to, or about one to ten nucleotides adjacent to a target sequence within a target nucleic acid. Additionally, the transgene may comprise a donor nucleic acid. Optionally, the AAV vector may have a third promoter (P3) and a second guide nucleic acid after the poly A signal sequence in a 5′ to 3′ direction. The second guide nucleic acid can be the same as the first guide nucleic acid where higher efficiency of cleaving is required. The second guide nucleic acid can be different from the first guide nucleic acid where cleaving is required at two different locations within, directly adjacent to, or about one to ten nucleotides adjacent to the target sequence. The effector protein has an amino acid sequence that is at least 90% identical to any one of the sequences recited TABLE 1. The guide nucleic acid comprises a spacer sequence that hybridizes to a portion of nucleotide sequence within the target nucleic acid, or a reverse complement thereof. Each of the promoters can be independently selected from ApoE, TBG, CMV, 7SK, EF1a, RPBSA, hPGK, EFS, SV40, PGK1, hPGK, Ube, human beta actin promoter, CAG, TRE, UAS, Ac5, Polyhedrin, CaMKIIa, GAL1-10, H1, TEF1, GDS, ADH1, CaMV35S, Ubi, U6, MNDU3, Ck8e, SPC5-12, Desmin, MND, and MSCV. The effector protein can be expressed either ubiquitously or in a specific tissue based on the promoter the AAV vector is engineered to have. In particular, the second promoter can be a tissue specific promoter or a ubiquitous promoter.

### Example 12: In Vivo Gene Editing in a Mammalian Model for Treating a Genetic Disorder by AAV

An AAV vector is constructed to contain a transgene between its ITRs according to any one of the constructs described in Examples 10, 11 and 12. The AAV vector is expressed with supporting plasmids to produce an adeno-associated virus (AAV). A mouse with a genetic disorder is administered an effective dose of the AAV. About four weeks post administration, a sample tissue is extracted for analysis of gene restoration. The sample tissue can be chosen based on the promoter used for expressing the effector protein. The analysis can be performed by any technique known to a skillful artisan, which includes but are not limited to immunohistochemistry, western blot analysis and deep-sequencing analysis. Similarly, rescue of pathological phenotypes can be determined by performing any technique known to a skillful artisan, which includes but are not limited to hematoxylin and eosin (H & E) staining, Masson's trichrome staining, grip-strength analysis, muscular electrophysiological analysis, and serum creatine kinase (CK).

### Example 13: Gene Editing of Eukaryotic Cells with AAV Vector Encoding Effector Protein and Guide RNA

An AAV vector is constructed to contain a transgene between its ITRs according to any one of the constructs described in Examples 10, 11 and 12. The AAV vector is expressed with supporting plasmids to produce an adeno-associated virus (AAV). Primary T cells are contacted with the AAV for 24 hours. After about 96 hours, post AAV contact, DNA or RNA is isolated from the infected T cells. An indel caused by the guide nucleic acid is confirmed by sequencing and/or Q-PCR.

### Example 14: Gene Editing of Eukaryotic Cells with scAAV Vector Encoding CasM.19952 and Guide RNA

An scAAV vector is constructed to contain a transgene between its ITRs, the transgene providing or encoding, in a 5′ to 3′ direction, a U6 promoter, a guide RNA, an EFS promoter, a Cas effector protein, and a SV40 poly A tail as illustrated in FIG. 8C. The Cas effector comprises a sequence of SEQ ID NO: 23. The guide RNA that are used for gene editing includes SEQ ID Nos: 329, 343, 354-355, 357-358, 360-361, 366, and 364. The AAV vector is expressed with supporting plasmids to produce an adeno-associated virus (AAV). Eukaryotic cells are contacted with the AAV for 24 hours. After about 96 hours, post AAV contact, DNA or RNA is isolated from the infected eukaryotic cells. An indel caused by the guide nucleic acid is confirmed by sequencing and/or Q-PCR. TABLE 9 recites amplicons that are sequenced for measuring indel activity with a specific guide RINA.

TABLE 9
Amplicon Sequences used with sgRNA in Primary T Cells

| sgRNA | |
|---|---|
| (SEQ ID NO: | Amplicon sequence |
| 329 | ACACAGACACCATCAACTGCGACCAGTTCAGCAGGCTGTTGTGTGACAT |
| | GGAAGGTGATGAAGAGACCAGGGAGGCTTATGCCAATATCGGTGAGGA |
| | AGCACCTGAGCCCAGAAAAGGACAATCAAGGGCAAGAGTTCTTTGCTG |
| | CCACTTGTCAATATCACCCATTCATCATGAGCCACGT (SEQ ID NO: 463) |
| 343 | TCTAGGGATGGTGGCTTCTGGAAGGCTGACCATGCACAGGCCTCCAATC |
| | CCTCCCCCTGGCCTCTGTTTCCGACAGCTTGTACAATAACTGCATCTGCG |
| | ACGTGGGAGCCGAGAGCTTGGCTCGTGTGCTTCCGGACATGGTGTCCCT |
| | CCGGGTGATGGAGTGAGTGTGGGAGTCTGGGCGGTGGGTGGCTCAGCCC |
| | GGGGTGGGAGACACTGAAGTCTCTCCCTGGTGTC (SEQ ID NO: 464) |
| 354 | GGATGGGAAGGGTCAGATGGCCCCAGGACGCTAGCTGATGGCCCCCAT |
| | CTGATTCCACCTGCAGCCTGGATGCGCTGAGTGAGAACAAGATCGGGGA |
| | CGAGGGTGTCTCGCAGCTCTCAGCCACCTTCCCCCAGCTGAAGTCCTTG |
| | GAAACCCTCAAGTGAGTGAGCTGGGCCTGCCCTTCCTGCTGAATCGGGC |
| | CCCCAAAGTCCGGCTGACTTTTTCAAAATTAATTTAAATTTGTTTTTTTA |
| | GACAAGGGCTCGCTG (SEQ ID NO: 465) |
| 355 | GCCCAAGAACTAGGAGGTCTGGGGTGGGAGAGTCAGCCTGCTCTGGAT |
| | GCTGAAAGAATGTCTGTTTTTCCTTTTAGAAAGTTCCTGTGATGTCAAGC |
| | TGGTCGAGAAAAGCTTTGAAACAGGTAAGACAGGGGTCTAGCCTGGGTT |
| | TGCACAGGATTGCGGAAGTGATGAACCCGCAATAACCCTGCCTGGATGA |
| | GGGAGTGGGAAGAAATTAGTAGATGTGGGAATGAATGATGAGGAATGG |
| | AAACAGCGGTT (SEQ ID NO: 466) |
| 357 | AGGGGATATGCACAGAAGCTGCAAGGGACAGGAGGTGCAGGAGCTGCA |
| | GGCCTCCCCCACCCAGCCTGCTCTGCCTTGGGGAAAACCGTGGGTGTGT |
| | CCTGCAGGCCATGCAGGCCTGGGACATGCAAGCCCATAACCGCTGTGGC |
| | CTCTTGGTTTTACAGATACGAACCTAAACTTTCAAAACCTGTCAGTGATT |
| | GGGTTCCGAATCCTCCTCCTGAAAGTGGCCGGGTTTAATCTGCTCATGAC |
| | GCTGC (SEQ ID NO: 467) |
| 358 | AGGGGATATGCACAGAAGCTGCAAGGGACAGGAGGTGCAGGAGCTGCA |
| | GGCCTCCCCCACCCAGCCTGCTCTGCCTTGGGGAAAACCGTGGGTGTGT |
| | CCTGCAGGCCATGCAGGCCTGGGACATGCAAGCCCATAACCGCTGTGGC |
| | CTCTTGGTTTTACAGATACGAACCTAAACTTTCAAAACCTGTCAGTGATT |
| | GGGTTCCGAATCCTCCTCCTGAAAGTGGCCGGGTTTAATCTGCTCATGAC |
| | GCTGC (SEQ ID NO: 468) |
| 360 | AATATAAGTGGAGGCGTCGCGCTGGCGGGCATTCCTGAAGCTGACAGCA |
| | TTCGGGCCGAGATGTCTCGCTCCGTGGCCTTAGCTGTGCTCGCGCTACTC |
| | TCTCTTTCTGGCCTGGAGGCTATCCAGCGTGAGTCTCTCCTACCCTCCCG |
| | CTCTGGTCCTTCCTCTCCCGCTCTGCACCCTCTGTGGCCCTCGCTGTGCTC |
| | TCTCGCTCCGTGACTTCCCTTCTCC (SEQ ID NO: 469) |
| 361 | CCCAAGTGAAATACCCTGGCAATATTAATGTGTCTTTTCCCGATATTCCT |
| | CAGGTACTCCAAAGATTCAGGTTTACTCACGTCATCCAGCAGAGAATGG |
| | AAAGTCAAATTTCCTGAATTGCTATGTGTCTGGGTTTCATCCATCCGACA |
| | TTGAAGTTGACTTACTGAAGAATGGAGAGAGAATTGAAAAAGTGGAGC |
| | ATTCAGACTTGTCTTTCAGCAAGGACTGGTCTTTCTATCTCTTGTACTAC |
| | ACTGAATTCACCCCCACTG (SEQ ID NO: 470) |
| 366 | AGCCTATTCTGCCAGCCTTATTTCTAACCATTTTAGACATTTGTTAGTAC |
| | ATGGTATTTTAAAAGTAAAACTTAATGTCTTCCTTTTTTTTCTCCACTGTC |
| | TTTTTCATAGATCGAGACATGTAAGCAGCATCATGGAGGTAAGTTTTTG |
| | ACCTTGAGAAAATGTTTTTGTTTCACTGTCCTGAGGACTATTTATAGACA |
| | GCTCTAACATGATAACCCTCACTATGTGGAGAACAT (SEQ ID NO: 471) |
| 364 | CCTCTCTCTAACCTGGCACTGCGTCGCTGGCTTGGAGACAGGTGACGGT |
| | CCCTGCGGGCCTTGTCCTGATTGGCTGGGCACGCGTTTAATATAAGTGG |
| | AGGCGTCGCGCTGGCGGGCATTCCTGAAGCTGACAGCATTCGGGCCGAG |
| | ATGTCTCGCTCCGTGGCCTTAGCTGTGCTCGCGCTACTCTCTCTTTCTGG |
| | CCTGGAGGCTATCCAGCGTGAGTCTCTCCTACCCTC (SEQ ID NO: 472) |

### Example 15: Gene Editing of Primary T Cells with scAAV Vector Encoding CasM.19952 and Guide RNA

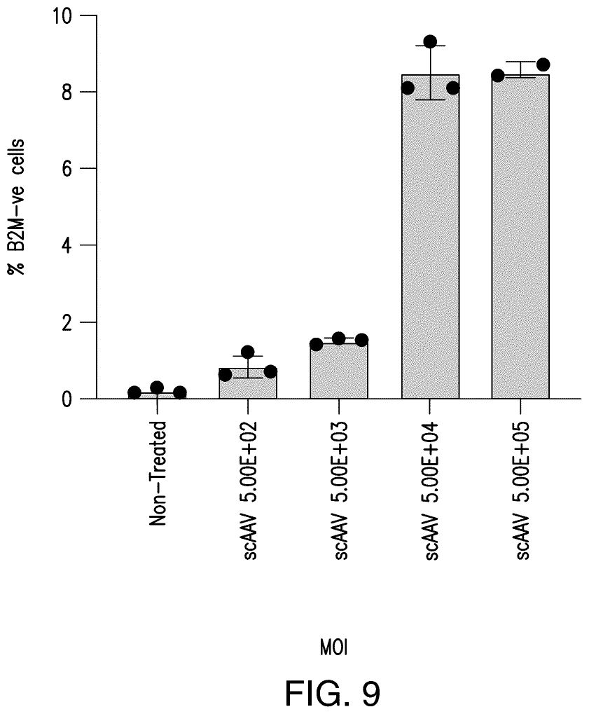

A dose response experiment for scAAV plasmid for testing its ability to produce indels in primary T cells was conducted. Briefly, a scAAV plasmid was constructed to contain a transgene between its ITRs, the transgene providing or encoding, in a 5′ to 3′ direction, a U6 promoter, a guide RNA, an EFS promoter, a Cas effector protein, and a SV40 poly A tail. The Cas effector protein was CasM.19952 (SEQ ID NO: 23). The guide RNA had a nucleotide sequence of SEQ ID No: 364. The scAAV vector was expressed with supporting plasmids to produce an adeno-associated virus (AAV). Activated primary T cells were transduced with the AAV at various concentrations (0, 5e+02, 5e+03, 5e+04, and 5e+05 GC/cell). About 96 hours post transduction, DNA or RNA was isolated from the infected cells. An indel caused by the guide nucleic acid was confirmed by sequencing and/or Q-PCR using amplicon SEQ ID NO: 472. Results of the dose response experiment are summarized in FIG. 9. An analysis of FIG. 9 indicates that AAV can be used to edit genes in primary T cells.

### Example 16: PROSITE Motifs of Effector Protein

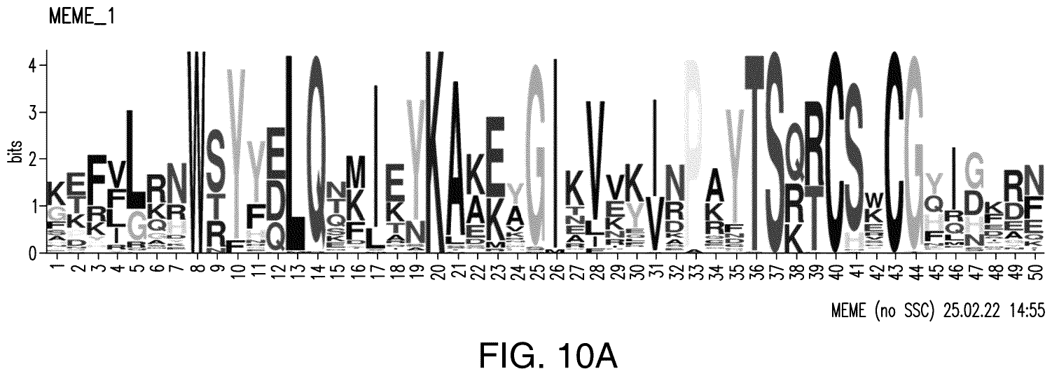

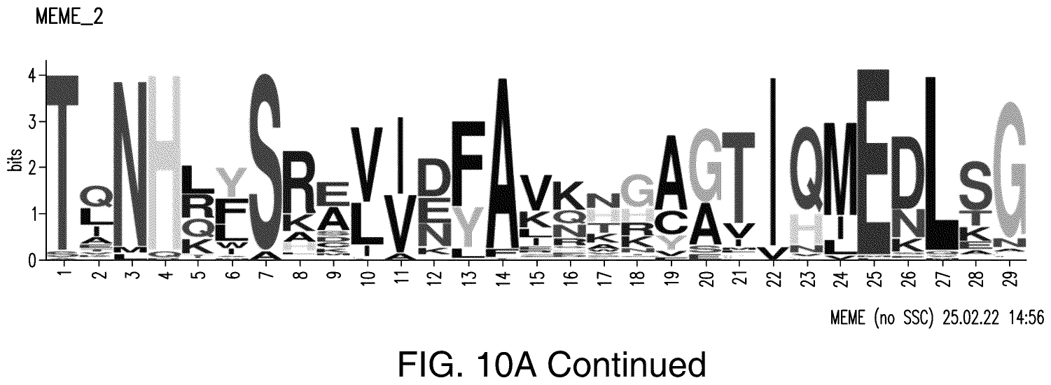

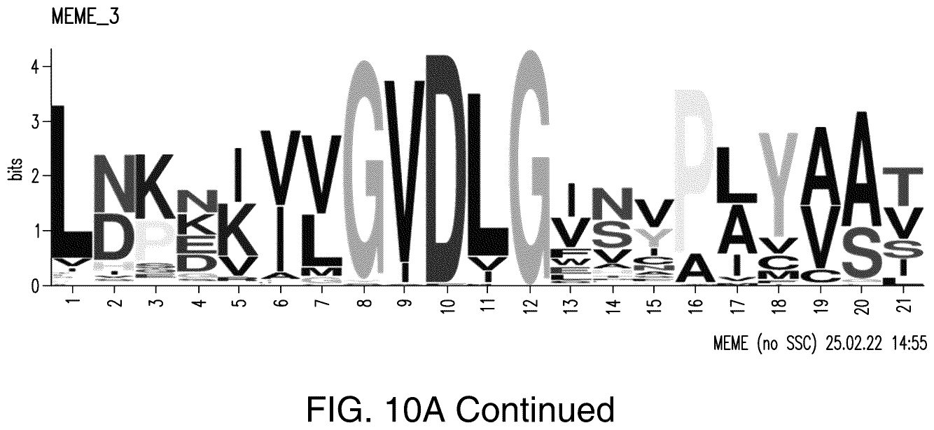

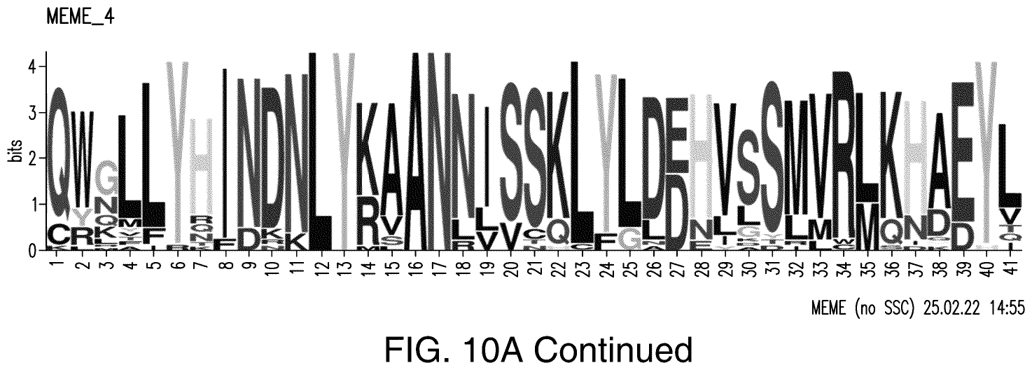

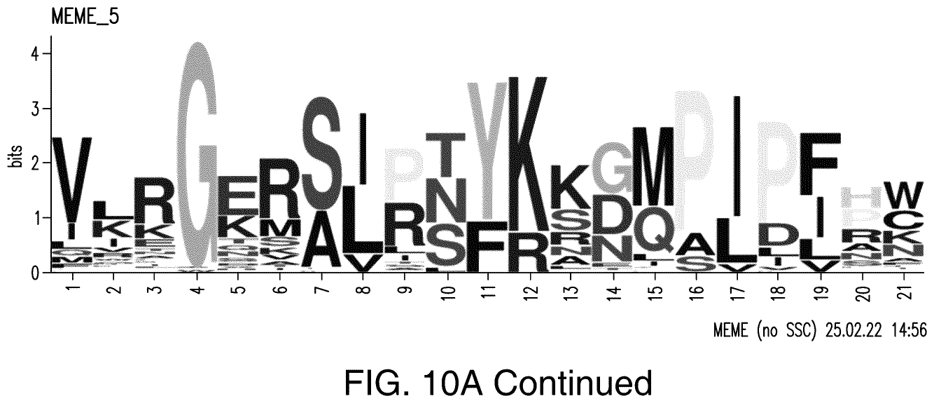

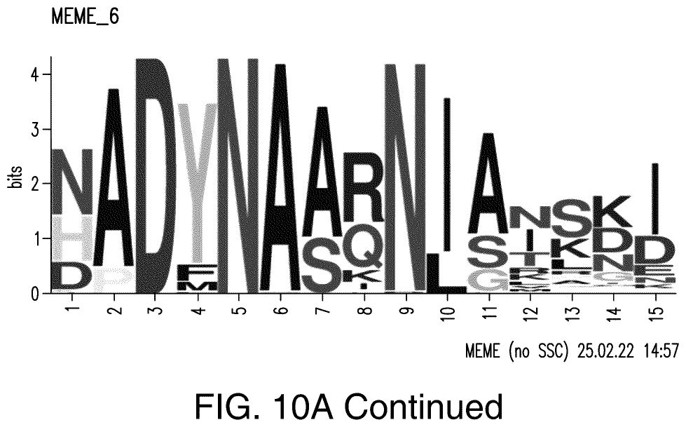

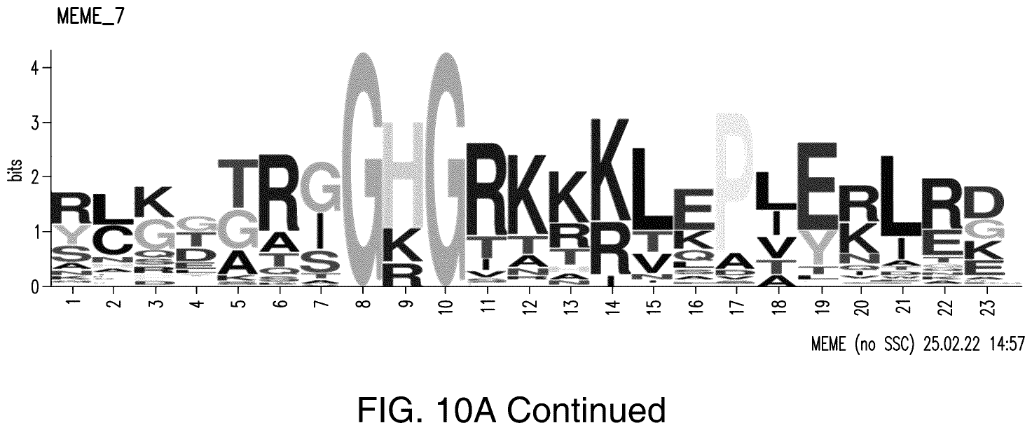

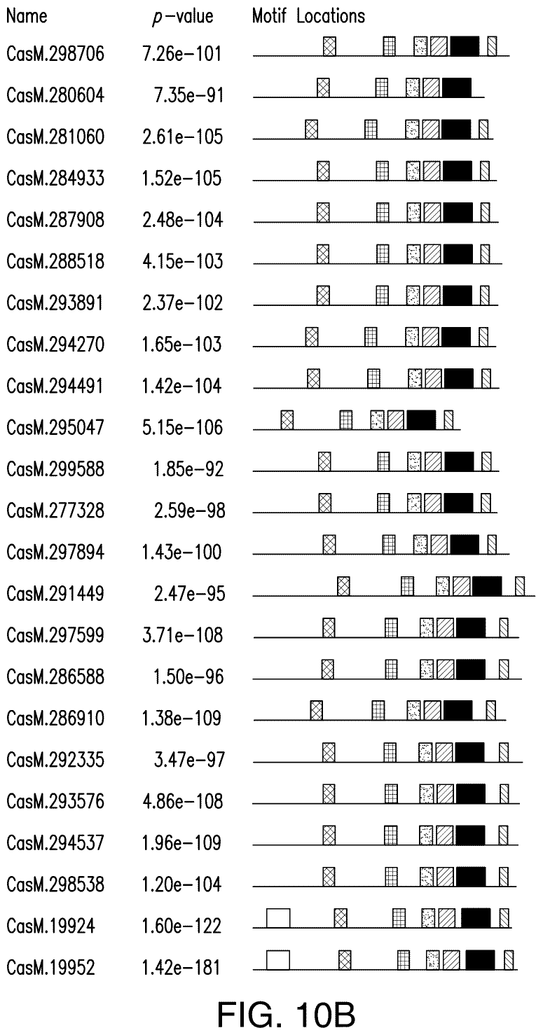

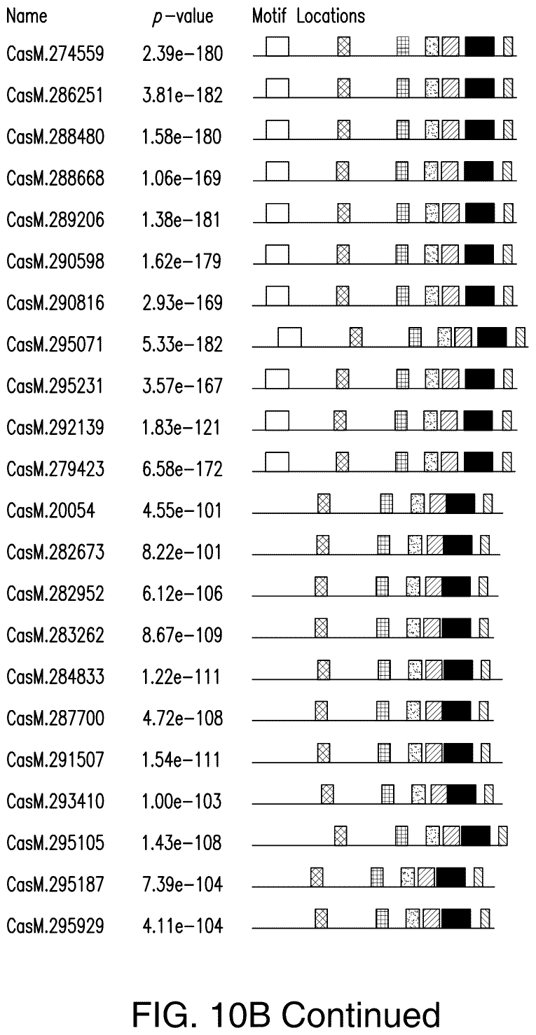

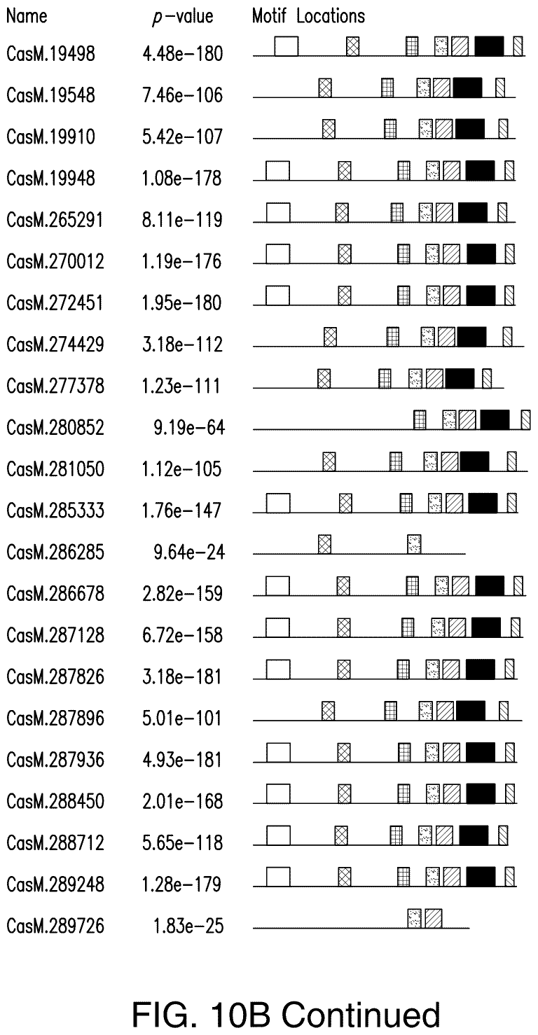

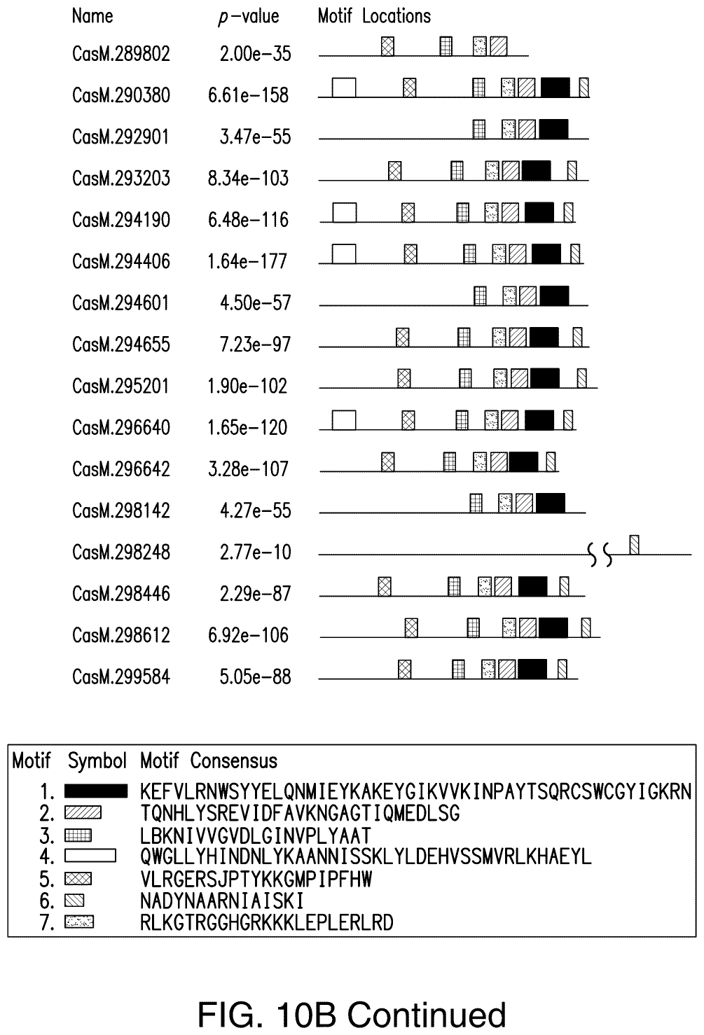

The MEME algorithm (Multiple EM for Motif Elicitation, Bailey & Elkan, 1994) was used to identify sequence motifs that are shared by any one of effector proteins of SEQ ID Nos: 1-45, 105-107, 109, 405-413, 415-428, 430-432, 434-437, and 439-443. The analysis was performed using the default parameters. This analysis identified the seven highly conserved motifs that are shown in FIG. 10A. The number of analyzed sequences that include the motifs is provided in TABLE 10 along with the length of each motif.

TABLE 10
PROSITE motif analysis

| | Number of sequences that | Motif |
|---|---|---|
| Motif ID | include the motif (out of 84) | length |
| MEME_1 | 79 | 50 |
| MEME_2 | 81 | 29 |
| MEME_3 | 80 | 21 |
| MEME_4 | 30 | 41 |
| MEME_5 | 77 | 21 |
| MEME_6 | 76 | 15 |
| MEME_7 | 82 | 23 |

The weblogos in FIG. 10A provide multilevel consensus sequences. Weblogos corresponding to MEME 1, MEME 2, MEME 3, MEME 4, MEME 5, MEME 6 and MEME 7 are shown in FIG. 10A. This multilevel sequence analysis of the weblogos in FIG. 10A was used to generate the PROSITE motifs shown in TABLE 11. In TABLE 11, the brackets indicate amino acids in the alternative, for example [KG] means K or G. In another example [VFL] means V, F, or L. PROSITE motifs are routinely used in the art to conveniently illustrate consensus motifs.

TABLE 11
PROSITE motifs

| Motif ID | PROSITE motif | SEQ ID NO |
|---|---|---|
| MEME_1 | [KG][ET]F[VFL][LG][RK]NW[SRT]Y[YF][EDQ]LQ[NT][MK]I[EK] | 448 |
| | YKA[KA]E[YA]GIKV[VE][KY][IV][NR]P[AK]YTS[QRK][RT]CS[WK] | |
| | CG[YQH]I[GD][KF][RD][NF] | |
| MEME_2 | T[QL]NH[LRQ][YF]SR[EA][VL][IV][DEN][FY]AVK[NH]GA[GA]T | 449 |
| | I[QH]ME[DN]LSG | |
| MEME_3 | L[ND][KP][NKE][IK][VI][VL]GVDLG[IV][NS][VY]P[LA]Y[AV] | 450 |
| | [AS][TV] | |
| MEME_4 | QW[GN]LLYHINDNLY[KRJAANNISSKLYLD[DE]HVSSMVR[LM] | 451 |
| | KH[AD]EYL | |
| MEME_5 | V[LK]RG[EK]R[SA][IL][PR][NTS][YF][KR][KS][GDN][MQ]P[IL]P | 452 |
| | [FI][HP][WC] | |
| MEME_6 | [NH]ADYNA[AS][RQIN[IL][AS][IN][SK][KD][ID] | 453 |
| MEME_7 | [RY][LC][GK][GT][TG]R[GI]G[HK]GRK[KR][KR]LEP[LI][EY][RK] | 454 |
| | L[RE][DG] | |

The location of the detected motifs in the effector proteins is illustrated in FIG. 10B. All motifs illustrated in FIG. 10B shared at least 36.5% identity to the PROSITE sequences shown in TABLE 11. In general, MEME_4 and MEME_5 are located in the N terminal half of the effector protein. In general, MEME_1, MEME_2, MEME_6, and MEME_7 are located in the C terminal half of the effector protein. In general, the order of MEMEs from N terminus to C terminus is: MEME_4, MEME_5, MEME_3, MEME 7, MEME 2, MEME 1, MEME_6.

In general, the motifs demonstrate a similar distribution in all domains of effector proteins shown in FIG. 10B, namely MEME_4, MEME_5, MEME_3, MEME_7, MEME_2, MEME_1 and MEME_6 (from N- to C-terminus). All seven motifs were identified in a lot of the effector proteins shown in FIG. 10B. However, all seven motifs are not always identified in the effector proteins. For example, in some instances, MEME_4 was not identified, but the effector protein includes MEME_5, MEME_3, MEME_7, MEME_2, MEME_1 and MEME_6 (from N- to C-terminus) e.g. for CasM.298706 (SEQ ID NO: 1).

The degree of identity of PROSITE motifs MEME_1 to MEME_7 in the effector proteins that share greater than 75% identity with CasM.19952 was calculated. In calculating these degrees of identity, each alternative in a prosite motif was given an equal weight. For example, both NAD or HAD share 100% identity with the prosite motif [NH] AD. The output from this identity analysis is shown in TABLE 12.

TABLE 12
Conservation of the PROSITE Motifs

| | Effector | | | | | | | |
|---|---|---|---|---|---|---|---|---|
| | Protein | | | | | | | |
| Effector | SEQ ID | MEME | | | | | | |
| Protein | NO: | 1 | 2 | 3 | 4 | 5 | 6 | 7 |
| CasM.19498 | 405 | 90.00 | 82.76 | 100.00 | 100.00 | 95.24 | 100.00 | 91.30 |
| CasM.19948 | 408 | 92.00 | 86.21 | 95.24 | 92.68 | 100.00 | 100.00 | 86.96 |
| CasM.19952 | 445 | 92.00 | 93.10 | 95.24 | 100.00 | 95.24 | 100.00 | 82.61 |
| CasM.270012 | 410 | 88.00 | 82.76 | 95.24 | 100.00 | 100.00 | 100.00 | 86.96 |
| CasM.272451 | 411 | 88.00 | 93.10 | 95.24 | 100.00 | 100.00 | 93.33 | 86.96 |
| CasM.274559 | 24 | 94.00 | 93.10 | 90.48 | 100.00 | 95.24 | 93.33 | 86.96 |
| CasM.279423 | 34 | 88.00 | 82.76 | 100.00 | 95.12 | 100.00 | 93.33 | 82.61 |
| CasM.286251 | 444 | 94.00 | 93.10 | 95.24 | 97.56 | 100.00 | 93.33 | 82.61 |
| CasM.287826 | 420 | 90.00 | 89.66 | 100.00 | 97.56 | 95.24 | 100.00 | 86.96 |
| CasM.287936 | 422 | 94.00 | 93.10 | 95.24 | 97.56 | 100.00 | 93.33 | 78.26 |
| CasM.288450 | 423 | 92.00 | 72.41 | 95.24 | 90.24 | 95.24 | 86.67 | 91.30 |
| CasM.288480 | 26 | 92.00 | 93.10 | 100.00 | 97.56 | 95.24 | 100.00 | 86.96 |
| CasM.288668 | 27 | 94.00 | 86.21 | 95.24 | 97.56 | 100.00 | 93.33 | 69.57 |
| CasM.289206 | 28 | 88.00 | 89.66 | 100.00 | 100.00 | 100.00 | 93.33 | 91.30 |
| CasM.289248 | 425 | 88.00 | 86.21 | 100.00 | 100.00 | 100.00 | 93.33 | 91.30 |
| CasM.290598 | 29 | 90.00 | 89.66 | 95.24 | 95.12 | 100.00 | 100.00 | 82.61 |
| CasM.290816 | 30 | 94.00 | 79.31 | 95.24 | 92.68 | 95.24 | 100.00 | 82.61 |
| CasM.294406 | 432 | 92.00 | 93.10 | 95.24 | 97.56 | 95.24 | 86.67 | 82.61 |
| CasM.295071 | 31 | 94.00 | 93.10 | 95.24 | 97.56 | 100.00 | 93.33 | 82.61 |
| CasM.295231 | 32 | 92.00 | 79.31 | 95.24 | 92.68 | 95.24 | 100.00 | 86.96 |

TABLE 12 shows that motifs MEME_1 to MEME_7 are highly conserved between effector proteins that are at least 75% identical to CasM.19952. In particular, all effector proteins described in TABLE 12 comprise an amino acid sequence that is at least 69.5% or more identical to each of MEME_1 to MEME_7. All effector proteins described in TABLE 12 comprise an amino acid sequence that is at least 72% identical to each of MEME_1 to MEME_6. All effector proteins described in Table 33 comprise an amino acid sequence that is at least 90% identical to each of MEME_1, and MEME_3 to MEME_6.

MEME_4 was found to be a particularly useful motif for identifying the group of effector proteins and distinguishing these effector proteins from previously known effector proteins. All effector proteins described in TABLE 12 comprise an amino acid sequence that is at least 90% identical to MEME_4. In some cases, the effector proteins include an amino acid sequence that is at least 37% identical to MEME_4.

### Example 17: Effector Protein Sequence Similarity

The following method was used to calculate the similarity of effector proteins (SEQ ID Nos 1-45, 105-107, 109, 405-413, 415-428, 430-432, 434-437, and 439-443) to CasM.19952, as well as the similarity of sequences within each effector protein sequence to the multilevel consensus sequence/PROSITE motifs described in Example 16.

The BLOSUM62 similarity matrix (Henikoff & Henikoff, 1992) was transformed so that any value ≥1 was replaced with +1 and any value ≤0 was replaced with 0. For example, the Ile to Leu substitution is scored at +2.0; in the transformed matrix, it is scored at +1. This transformation allows the calculation of percent similarity, rather than a similarity score.

For similarity over the MEME motifs, the multilevel consensus sequence (or PROSITE motif sequence) was used to identify how strongly each motif was conserved. In calculating the similarity of a motif sequence, the second and third levels of the multilevel sequence were treated as equivalent to the top level. Alternately, when comparing two full protein sequences, the proteins were aligned using pairwise MUSCLE alignment. Then, the similarity was scored at each residue and divided by the length of the alignment.

If a substitution could be treated as conservative with any of the amino acids in that position of the multilevel consensus sequence, +1 point was assigned. For example, given the multilevel consensus sequence:
RLG
YCK
. . . the test sequence QIQ would receive three points. This is because in the transformed BLOSUM62 matrix, each combination is scored as: Q-R: +1; Q-Y: +0; I-L: +1; I-C: +0; Q-G: +0; Q-K: +1 For each position, the highest score is used when calculating similarity.

The score over the length of the motif was divided by the length of the motif to provide the % similarity. In the example above, the % similarity would be 100%. This process is equivalent to the percent similarity calculation used by the Geneious Prime software given the parameters matrix=BLOSUM62 and threshold ≥1.

As shown in TABLE 13, there are 24 effector proteins with greater than 70% similarity to CasM.19952. Including CasM.19952, there are 26 sequences that have greater than 80% similarity to six or more of the MEME motifs, as shown int TABLE 14. Of these, 19 (excluding CasM.19952 itself) have greater than 80% similarity to the MEME motifs of CasM.19952. These are the same 19 sequences with at least 75% identity to CasM.19952 overall.

TABLE 13
Effector Protein Sequence Similarity

| Effector Protein | SEQ ID NO: | % similarity to CasM.19952 |
|---|---|---|
| CasM.19952 | 23 | 100.0 |
| CasM.288480 | 26 | 97.9 |
| CasM.274559 | 24 | 96.8 |
| CasM.272451 | 411 | 95.9 |
| CasM.289206 | 28 | 92.3 |
| CasM.289248 | 425 | 92.3 |
| CasM.290598 | 29 | 92.9 |
| CasM.287826 | 420 | 92.1 |
| CasM.294406 | 432 | 89.8 |
| CasM.286251 | 25 | 90.2 |
| CasM.290816 | 30 | 90.4 |
| CasM.287936 | 422 | 90.0 |
| CasM.270012 | 410 | 91.2 |
| CasM.295231 | 32 | 90.9 |
| CasM.19498 | 405 | 87.4 |
| CasM.288450 | 423 | 90.8 |
| CasM.19948 | 408 | 88.5 |
| CasM.279423 | 34 | 89.5 |
| CasM.295071 | 31 | 86.4 |
| CasM.288668 | 27 | 86.1 |
| CasM.285333 | 416 | 78.0 |
| CasM.290380 | 428 | 76.3 |
| CasM.287128 | 419 | 76.0 |
| CasM.286678 | 418 | 75.7 |
| CasM.19924 | 22 | 71.6 |
| CasM.292139 | 33 | 65.1 |
| CasM.265291 | 409 | 58.9 |
| CasM.296640 | 436 | 60.4 |
| CasM.288712 | 424 | 59.0 |
| CasM.294190 | 431 | 57.5 |
| CasM.299584 | 443 | 57.0 |
| CasM.298446 | 441 | 50.6 |

TABLE 14
MEME motif percent similarity

| | Effector | | | | | | | |
|---|---|---|---|---|---|---|---|---|
| | Protein | MEME | | | | | | |
| Effector Protein | SEQ ID NO: | 1 | 2 | 3 | 4 | 5 | 6 | 7 |
| CasM.19952 | 23 | 96.0 | 96.6 | 100.0 | 100.0 | 95.2 | 80.0 | 87.0 |
| CasM.288480 | 26 | 96.0 | 96.6 | 100.0 | 100.0 | 95.2 | 80.0 | 87.0 |
| CasM.274559 | 24 | 94.0 | 93.1 | 95.2 | 100.0 | 95.2 | 80.0 | 87.0 |
| CasM.272451 | 411 | 94.0 | 93.1 | 100.0 | 100.0 | 100.0 | 80.0 | 87.0 |
| CasM.289206 | 28 | 92.0 | 93.1 | 100.0 | 100.0 | 100.0 | 100.0 | 91.3 |
| CasM.289248 | 425 | 94.0 | 93.1 | 100.0 | 100.0 | 100.0 | 100.0 | 91.3 |
| CasM.290598 | 29 | 96.0 | 93.1 | 100.0 | 100.0 | 100.0 | 76.7 | 82.6 |
| CasM.287826 | 420 | 96.0 | 93.1 | 100.0 | 100.0 | 100.0 | 100.0 | 87.0 |
| CasM.294406 | 432 | 94.0 | 96.6 | 95.2 | 97.6 | 95.2 | 93.3 | 87.0 |
| CasM.286251 | 25 | 94.0 | 93.1 | 95.2 | 97.6 | 100.0 | 100.0 | 87.0 |
| CasM.290816 | 30 | 96.0 | 89.7 | 100.0 | 95.1 | 95.2 | 100.0 | 91.3 |
| CasM.287936 | 422 | 94.0 | 93.1 | 95.2 | 97.6 | 100.0 | 100.0 | 87.0 |
| CasM.270012 | 410 | 92.0 | 93.1 | 100.0 | 100.0 | 100.0 | 100.0 | 87.0 |
| CasM.295231 | 32 | 96.0 | 89.7 | 100.0 | 95.1 | 95.2 | 100.0 | 87.0 |
| CasM.19498 | 405 | 92.0 | 93.1 | 100.0 | 100.0 | 100.0 | 100.0 | 91.3 |
| CasM.288450 | 423 | 92.0 | 89.7 | 100.0 | 95.1 | 95.2 | 86.7 | 91.3 |
| CasM.19948 | 408 | 94.0 | 93.1 | 100.0 | 95.1 | 100.0 | 100.0 | 87.0 |
| CasM.279423 | 34 | 96.0 | 93.1 | 100.0 | 97.6 | 100.0 | 100.0 | 87.0 |
| CasM.295071 | 31 | 94.0 | 93.1 | 95.2 | 97.6 | 100.0 | 100.0 | 87.0 |
| CasM.288668 | 27 | 94.0 | 93.1 | 95.2 | 97.6 | 100.0 | 93.3 | 91.3 |
| CasM.285333 | 416 | 64.0 | 86.2 | 95.2 | 92.7 | 95.2 | 93.3 | 91.3 |
| CasM.290380 | 428 | 80.0 | 89.7 | 100.0 | 92.7 | 90.5 | 86.7 | 95.7 |
| CasM.287128 | 419 | 80.0 | 86.2 | 100.0 | 95.1 | 95.2 | 93.3 | 95.7 |
| CasM.286678 | 418 | 82.0 | 89.7 | 100.0 | 92.7 | 95.2 | 86.7 | 95.7 |
| CasM.19924 | 22 | 100.0 | 86.2 | 90.5 | 68.3 | 90.5 | 86.7 | 91.3 |
| CasM.292139 | 33 | 86.0 | 82.8 | 90.5 | 61.0 | 90.5 | 93.3 | 95.7 |
| CasM.265291 | 409 | 74.0 | 72.4 | 76.2 | 68.3 | 85.7 | 80.0 | 69.6 |
| CasM.296640 | 436 | 76.0 | 75.9 | 85.7 | 53.7 | 85.7 | 80.0 | 73.9 |
| CasM.294190 | 431 | 74.0 | 69.0 | 76.2 | 70.7 | 71.4 | 80.0 | 69.6 |
| CasM.288712 | 424 | 78.0 | 75.9 | 85.7 | 52.4 | 81.0 | 80.0 | 73.9 |
| CasM.299584 | 443 | 84.0 | 69.0 | 85.7 | 58.5 | 95.2 | 80.0 | 69.6 |
| CasM.298446 | 441 | 84.0 | 86.2 | 76.2 | | 71.4 | 93.3 | 73.9 |
| CasM.289802 | 427 | | 58.6 | 61.9 | 43.9 | 66.7 | | 60.9 |
| CasM.286285 | 417 | | 58.6 | | 53.7 | 81.0 | | 69.6 |
| CasM.20054 | 35 | 88.0 | 82.8 | 90.5 | | 76.2 | 93.3 | 78.3 |
| CasM.284933 | 4 | 80.0 | 89.7 | 85.7 | | 76.2 | 93.3 | 73.9 |
| CasM.289726 | 426 | | 58.6 | 57.1 | 46.3 | 61.9 | | 65.2 |
| CasM.294537 | 20 | 88.0 | 79.3 | 95.2 | | 81.0 | 80.0 | 69.6 |
| CasM.295929 | 45 | 86.0 | 82.8 | 90.5 | | 76.2 | 93.3 | 78.3 |
| CasM.298538 | 21 | 82.0 | 75.9 | 95.2 | 41.5 | 81.0 | 80.0 | 73.9 |
| CasM.286588 | 16 | 82.0 | 79.3 | 76.2 | 36.6 | 76.2 | 86.7 | 65.2 |
| CasM.19910 | 407 | 90.0 | 75.9 | 95.2 | | 81.0 | 86.7 | 69.6 |
| CasM.291449 | 14 | 84.0 | 75.9 | 90.5 | | 81.0 | 86.7 | 73.9 |
| CasM.293576 | 19 | 86.0 | 75.9 | 95.2 | | 85.7 | 80.0 | 65.2 |
| CasM.287896 | 421 | 84.0 | 82.8 | 90.5 | | 81.0 | 70.0 | 69.6 |
| CasM.293410 | 42 | 90.0 | 82.8 | 90.5 | 31.7 | 81.0 | 93.3 | 78.3 |
| CasM.295187 | 44 | 90.0 | 82.8 | 90.5 | 31.7 | 81.0 | 93.3 | 78.3 |
| CasM.297599 | 15 | 86.0 | 79.3 | 95.2 | | 85.7 | 86.7 | 63.0 |
| CasM.286910 | 17 | 88.0 | 79.3 | 95.2 | | 81.0 | 80.0 | 69.6 |
| CasM.296642 | 437 | 92.0 | 79.3 | 95.2 | | 71.4 | 93.3 | 73.9 |
| CasM.298612 | 442 | 82.0 | 79.3 | 95.2 | | 81.0 | 80.0 | 65.2 |
| CasM.274429 | 412 | 90.0 | 75.9 | 90.5 | 41.5 | 81.0 | 93.3 | 69.6 |
| CasM.282673 | 36 | 88.0 | 82.8 | 90.5 | | 85.7 | 86.7 | 78.3 |
| CasM.294601 | 107 | 72.0 | 72.4 | 76.2 | | | | 60.9 |
| CasM.294270 | 8 | 86.0 | 93.1 | 66.7 | | 76.2 | 100.0 | 65.2 |
| CasM.295105 | 43 | 90.0 | 89.7 | 95.2 | | 81.0 | 93.3 | 73.9 |
| CasM.19548 | 406 | 80.0 | 75.9 | 95.2 | 39.0 | 81.0 | 86.7 | 69.6 |
| CasM.287908 | 5 | 86.0 | 96.6 | 85.7 | | 81.0 | 93.3 | 78.3 |
| CasM.291507 | 41 | 86.0 | 86.2 | 95.2 | | 85.7 | 93.3 | 73.9 |
| CasM.283262 | 38 | 90.0 | 89.7 | 95.2 | | 81.0 | 93.3 | 73.9 |
| CasM.295201 | 435 | 88.0 | 79.3 | 95.2 | | 85.7 | 93.3 | 65.2 |
| CasM.284833 | 39 | 86.0 | 86.2 | 90.5 | | 81.0 | 93.3 | 73.9 |
| CasM.294655 | 434 | 88.0 | 89.7 | 90.5 | | 85.7 | 86.7 | 69.6 |
| CasM.277328 | 12 | 82.0 | 93.1 | 90.5 | | 85.7 | 93.3 | 73.9 |
| CasM.292335 | 18 | 84.0 | 82.8 | 85.7 | 41.5 | 85.7 | 80.0 | 69.6 |
| CasM.294491 | 9 | 86.0 | 93.1 | 85.7 | | 81.0 | 86.7 | 78.3 |
| CasM.293203 | 430 | 88.0 | 75.9 | 90.5 | | 90.5 | 86.7 | 73.9 |
| CasM.287700 | 40 | 88.0 | 89.7 | 95.2 | | 81.0 | 100.0 | 73.9 |
| CasM.280852 | 105 | 66.0 | 72.4 | 76.2 | | | 93.3 | 60.9 |
| CasM.293891 | 7 | 80.0 | 96.6 | 85.7 | | 81.0 | 93.3 | 69.6 |
| CasM.281060 | 3 | 84.0 | 93.1 | 66.7 | | 76.2 | 100.0 | 73.9 |
| CasM.299588 | 11 | 86.0 | 82.8 | 90.5 | | 76.2 | 100.0 | 69.6 |
| CasM.288518 | 6 | 82.0 | 93.1 | 90.5 | | 76.2 | 70.0 | 73.9 |
| CasM.280604 | 2 | 84.0 | 89.7 | 90.5 | | 85.7 | | 78.3 |
| CasM.298706 | 1 | 88.0 | 89.7 | 76.2 | | 71.4 | 93.3 | 73.9 |
| CasM.281050 | 415 | 88.0 | 75.9 | 95.2 | | 76.2 | 93.3 | 69.6 |
| CasM.277378 | 413 | 86.0 | 86.2 | 90.5 | | 81.0 | 93.3 | 73.9 |
| CasM.297894 | 13 | 88.0 | 89.7 | 76.2 | | 71.4 | 93.3 | 73.9 |
| CasM.295047 | 10 | 80.0 | 89.7 | 85.7 | | 76.2 | 93.3 | 73.9 |
| CasM.282952 | 37 | 88.0 | 89.7 | 85.7 | | 85.7 | 80.0 | 78.3 |
| CasM.298142 | 109 | 66.0 | 69.0 | 81.0 | | | | 60.9 |
| CasM.292901 | 106 | 72.0 | 72.4 | 59.5 | | | | 60.9 |
| CasM.298264 | 440 | | | 52.4 | | 38.1 | | |

### Example 18. Comparison of scAAV and Standard AAV Infection

A first population of CD34-positive cells are infected with scAAV carrying a sequence encoding GFP. A second population of CD34-positive cells are infected with a standard (non-scAAV) AAV vector also carrying a sequence encoding GFP. The dose of scAAV required to achieve similar expression of GFP as the standard AAV is expected to be lower than the dose of AAV.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising a self-complementary adeno-associated viral (scAAV) vector, wherein the scAAV vector comprises a transgene comprising:
a) a first region that encodes an effector protein; and
b) a second region that encodes a guide nucleic acid, wherein at least a portion of the guide nucleic acid is complementary to a target region of a target nucleic acid.

2. (canceled)

3. (canceled)

4. A composition comprising a viral vector, wherein the viral vector comprises a transgene comprising:
a) a first region that encodes an effector protein;
b) a second region that encodes a guide nucleic acid, wherein at least a portion of the guide nucleic acid is complementary to a target region of a target nucleic acid; and
c) a third region that comprises a donor nucleic acid.

5-65. (canceled)

66. An adeno-associated viral (AAV) vector comprising a transgene, wherein the transgene comprises:
a) a first nucleotide sequence encoding an effector protein;
b) a second nucleotide sequence encoding a first guide nucleic acid; and
c) a third nucleotide sequence encoding a second guide nucleic acid,
wherein the first guide nucleic acid and the second guide nucleic acid comprise different spacer sequences targeting different sequences in a target nucleic acid.

67. The AAV vector of claim 66 comprising:
a first promoter that drives expression of the first guide nucleic acid;
a second promoter that drives expression of the effector protein; and
a third promoter that drives expression of the second guide nucleic acid.

68. The AAV vector of claim 67, wherein any one of the promoters is a tissue specific promoter.

69. The AAV vector of claim 67, wherein the second promoter is a tissue specific promoter.

70. The AAV vector of claim 69, wherein the tissue specific promoter is a muscle specific promoter.

71. The AAV vector of claim 70, wherein the muscle specific promoter is selected from Ck8e, SPC5-12, and Desmin.

72. The AAV vector of claim 67, wherein the transgene encodes in a 5′ to 3′ direction, the first promoter, the first guide nucleic acid, the second promoter, the effector protein, an enhancer, a poly A signal, the third promoter, and the second guide nucleic acid.

73. The AAV vector of claim 67, wherein each of the promoters is independently selected from ApoE, TBG, CMV, 7SK, EF1a, RPBSA, hPGK, EFS, SV40, PGK1, hPGK, Ubc, human beta actin promoter, CAG, TRE, UAS, Ac5, Polyhedrin, CaMKIIa, GAL1-10, H1, TEF1, GDS, ADH1, CaMV35S, Ubi, U6, MNDU3, Ck8e, SPC5-12, Desmin, MND, and MSCV.

74. The AAV vector of claim 66, wherein the length of the effector protein is about 400 to about 600 amino acids.

75. The AAV vector of claim 66, wherein the effector protein comprises a RuvC domain but lacks an HNH domain.

76. The AAV vector of claim 66, wherein the transgene encodes at least one nuclear localization signal (NLS) fused to at least one of the N terminus and the C terminus of the effector protein.

77. The AAV vector of claim of claim 66, wherein the length of the first guide nucleic acid is less than 200 nucleotides and the length of the second guide nucleic acid is less than 200 nucleotides.

78. The AAV vector of claim 66, comprising a transcriptional enhancer, wherein the transcriptional enhancer comprises a Woodchuck Hepatitis Virus (WHV) posttranscriptional regulatory element (WPRE).

79. The AAV vector of claim 66, wherein the target nucleic acid is a gene selected from: AAVS1, ABCA4, ABCB11, ABCC8, ABCD1, ABCG5, ABCG8, ACAD9, ACADM, ACADVL, ACAT1, ACOX1, ACSF3, ADA, ADAMTS2, ADGRG1, AGA, AGL, AGPS, AGXT, AHI1, AIRE, ALDH3A2, ALDOB, ALG6, ALK, ALKBH5, ALMS1, ALPL, AMRC9, AMT, ANAPC10, ANAPC11, ANGPTL3, APC, Apo(a), APOCIII, APOEε4, APOL1, APP, AQP2, AR, ARFRP1, ARG1, ARH, ARL13B, ARL6, ARSA, ARSB, ASL, ASNS, ASPA, ASS1, ATM, ATP6V1B1, ATP7A, ATP7B, ATRX, ATXN1, ATXN10, ATXN2, ATXN3, ATXN7, ATXN8OS, AXIN1, AXIN2, B2M, BACE-1, BAK1, BAP1, BARD1, BAX2, BBS1, BBS10, BBS12, BBS2, BCKDHA, BCKDHB, BCL2L2, BCS1L, BEST1, Betaglobin gene, BLM, BMPR1A, BRAF, BRAFV600E, BRCA1, BRCA2, BRIP1, BSND, C90RF72, CA4, CACNA1A, CAH1, CAPN3, CASR, CBS, CCNB1 CC2D2A, CCR5, CD1, CD2, CD3, CD3D, CD3Z, CD4, CD5, CD6, CD7, CD8A, CD8B, CD9, CD14, CD18, CD19, CD21, CD22, CD23, CD27, CD28, CD30, CD33, CD34, CD36, CD38, CD40, CD40L, CD44, CD46, CD47, CD48, CD52, CD55, CD57, CD58, CD59, CD68, CD69, CD72, CD73, CD74, CD79A, CD80, CD81, CD83, CD84, CD86, CD90, CD93, CD96, CD99, CD100, CD123, CD160, CD163, CD164, CD164L2, CD166, CD200, CD204, CD207, CD209, CD226, CD244, CD247, CD274, CD276, CD300, CD320, CDC73, CDH1, CDH23, CDK 11, CDK4, CDKN1A, CDKN1B, CDKN1C, CDKN2A, CDKN2B, CEBPA, CELA3B, CEP290, CERKL, CFB, CFTR, CHCHD10, CHEK2, CHM, CHRNE, CIITA, CLN3, CLN5, CLN6, CLN8, CLRN1, CLTA, CMT1A, CNBP, CNGB1, CNGB3, COL1A1, COL1A2, COL27A1, COL4A3, COL4A4, COL4A5, COL7A1, CPS1, CPT1A, CPT2, CRB1, CREBBP, CRX, CRYAA, CTNNA1, CTNNB1, CTNND2, CTNS, CTSK, CXCL12, CYBA, CYBB, CYP11B1, CYP11B2, CYP17A1, CYP19A1, CYP21A2, CYP27A1, DBT, DCC, DCLRE1C, DERL2, DFNA36, DFNB31, DGAT2, DHCR7, DHDDS, DICER1, DIS3L2, DLD, DMD, DMPK, DNAH5, DNAI1, DNAI2, DNM2, DNMT1, DPC4, DYSF, EDA, EDN3, EDNRB, EGFR, EIF2B5, EMC2, EMC3, EMD, EMX1, EN1, EPCAM, ERCC6, ERCC8, ESCO2, ETFA, ETFDH, ETHE1, EVC, EVC2, EYS, F5, F8, F9, FXI, FAH, FAM161A, FANCA, FANCB, FANCC, FANCD1, FANCD2, FANCE, FANCF, FANCG, FANCI, FANCJ, FANCL, FANCM, FANCN, FANCP, FANCS, FBN1, FGF14, FGFR2, FGFR3, FGA, FGB, FGG, FH, FHL1, FIX, FKRP, FKTN, FLCN, FMR1, FOXP3, FSCN2, FSHD1, FUS, FUT8, FVIII, FXII, FXN, G6PC, GAA, GALC, GALK1, GALT, GAMT, GATA2, GATA-4, GBA, GBE1, GCDH, GCGR, GDNF, GFAP, GFM1, GHR, GJB1, GJB2, GLA, GLB1, GLDC, GLE1, GNE, GNPTAB, GNPTG, GNS, GPC3, GPR98, GREM1, GRHPR, GRIN2B, H2AFX, H2AX, HADHA, HAX1, HBA1, HBA2, HBB, HER2, HEXA, HEXB, HFE, HGSNAT, HLCS, HMGCL, HOGA1, HOXB13, HPRPF3, HPRT1, HPS1, HPS3, HRAS, HRD1, HSD17B4, HSD3B2, HTT, HUS1, HYAL1, HYLS1, IDS, IDUA, IFITM5, IKBKAP, IL2RG, IL7R, IMPDH1, INPP5E, IRF4, ITGB2, ITPR1, IVD, JAG1, JAK1, JAK3, KCNC3, KCND3, KCNJ11, KLHL7, KRAS, LAMA2, LAMA3, LAMB3, LAMC2, LCA5, LDLR, LDLRAP1, LHX3, LIFR, LIPA, LMNA, LOR, LOXHD1, LPL, LRAT, LRP6, LRPPRC, LRRK2, MADR2, MAN2B1, MAPT, MAX, MCM6, MCOLN1, MECP2, MED17, MEFV, MEN1, MERTK, MESP2, MET, METex14, MFN2, MFSD8, MIA3, MITF, MKL2, MKS1, MLC1, MLH1, MLH3, MMAA, MMAB, MMACHC, MMADHC, MMD, MPI, MPL, MPV17, MSH2, MSH3, MSH6, MTHFD1L, MTHFR, MTM1, MTRR, MTTP, MUT, MUTYH, MYC, MYH7, MYO7A, NAGLU, NAGS, NBN, NDRG1, NDUFAF5, NDUFS6, NEB, NF1, NF2, NKX2-5, NOG, NOTCH1, NOTCH2, NPC1, NPC2, NPHP1, NPHS1, NPHS2, NRAS, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9, PD1, PDCD1, PDE6B, PDGFRA, PDHA1, PDHB, PEX1, PEX10, PEX12, PEX13, PEX14, PEX16, PEX19, PEX2, PEX26, PEX3, PEX5, PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE, POMGNT1, POT1, POU5F1, PPM1A, PPP2R2B, PPT1, PRCD, PRKAG2, PRKAR1A, PRKCG, PRNP, PROM1, PROP1, PRPF31, PRPF8, PRPH2, PRPS1, PSAP, PSD95, PSEN1, PSEN2, PSRC1, PTCH1, PTEN, PTS, PUS1, PYGM, RAB23, RAD50, RAD51C, RAD51D, RAG1, RAG2, RAPSN, RARS2, RB1, RDH12, RECQL4, RET, RHO, RICTOR, RMRP, ROS1, RP1, RP2, RPE65, RPGR, RPGRIP1L, RPL32P3, RS1, RTCA, RTEL1, RUNX1, SACS, SAMHD1, SCN1A, SCN2A, SDHA, SDHAF2, SDHB, SDHC, SDHD, SEL1L, SEPSECS, SERPINA1, SERPINC1, SERPING1, SGCA, SGCB, SGCG, SGSH, SIRT1, SLC12A3, SLC12A6, SLC17A5, SLC22A5, SLC25A13, SLC25A15, SLC26A2, SLC26A4, SLC35A3, SLC35B4 SLC37A4, SLC39A4, SLC4A11, SLC6A8, SLC7A7, SMAD3, SMAD4, SMAD7, SMARCA4, SMARCAL1, SMARCB1, SMARCE1, SMN1, SMPD1, SNAI2, SNCA, SNRNP200, SOD1, SOX10, SPARA7, SPTBN2, STAR, STAT3, STK11, SUFU, SUMF1, SYNE1, SYNE2, SYS1, TARDBP, TAT, TBK1, TBP, TCIRG1, TCTN3, TECPR2, TERC, TERT, TFR2, TGFBR2, TGM1, TH, TLE3, TMEM127, TMEM138, TMEM216, TMEM43, TMEM67, TMPRSS6, TOP1, TOPORS, TP53, TPP1, TRAC, TRMU, TSC1, TSC2, TSFM, TSPAN14, TTBK2, TTC8, TTPA, TTR, TULP1, TYMP, UBE2G2, UBE2J1, UBE3A, USH1C, USH1G, USH2A, VEGF, VHL, VPS13A, VPS13B, VPS35, VPS45, VRK1, VSX2, VWF, WAS, WDR19, WDR48, WNT10A, WRN, WS2B, WS2C, WT1, XPA, XPC, XPF, XRCC3, YAP1, ZAC1, ZEB1, ZFYVE26, and ZNF423.

80. The AAV vector of claim 79, wherein the different sequences in the target nucleic acid are located on either side of an exon of the gene.

81. A method of modifying a target nucleic acid, the method comprising contacting the target nucleic acid with the AAV vector of claim 66 to cleave the nucleic acid at two different target sequences of the target nucleic acid, thereby modifying the target nucleic acid.

82. A cell modified by the AAV vector of claim 66.

83. An AAV virus comprising the AAV vector of claim 66.
